(19) United States
(12) Patent Application Publication
(10) Pub. No.: US 2026/0107684 A1
(43) Pub. Date: Apr. 16, 2026

(54) HETEROCYCLIC COMPOUND, ORGANIC LIGHT EMITTING DEVICE COMPRISING THE SAME AND COMPOSITION FOR ORGANIC MATERIAL LAYER

(71) Applicant: LT MATERIALS CO.,LTD., Yongin-si (KR)

(72) Inventors: Gi Back LEE, Yongin-si (KR); Jun Tae MO, Yongin-si (KR); Dong Jun KIM, Yongin-si (KR); Dae Hyuk CHOI, Yongin-si (KR)

(21) Appl. No.: 19/342,584

(22) Filed: Sep. 28, 2025

(30) Foreign Application Priority Data
Oct. 16, 2024 (KR) 10-2024-0141108

(51) Int. Cl. H10K 85/60; C07D 209/80; C07D 307/77; C07D 333/50; C07D 405/12; C07D 409/12; C09K 11/02; H10K 50/11; H10K 50/15; H10K 50/18

(52) U.S. Cl. CPC H10K 85/636; C07D 209/80; C07D 307/77; C07D 333/50; C07D 405/12; C07D 409/12; C09K 11/02; H10K 85/633; H10K 85/654; H10K 50/11; H10K 50/15; H10K 50/181; H10K 85/615; H10K 85/624; H10K 85/6572; H10K 85/6574; H10K 85/6576

(57) ABSTRACT

Provided is a heterocyclic compound represented by Chemical Formula 1, an organic light emitting device including the same, and a composition for an organic material layer. When the heterocyclic compound represented by Chemical Formula 1 is used in an organic material layer, it is possible to lower a driving voltage of an organic light emitting device, and improve light emission efficiency and lifetime properties thereof.

### TECHNICAL FIELD

This application claims priority to Korean Patent Application No. 10-2024-0141108, filed on Oct. 16, 2024, the disclosure of which is incorporated herein by reference in its entirety.

The present disclosure relates to a heterocyclic compound, an organic light emitting device comprising the same, and a composition for an organic material layer.

### DESCRIPTION OF THE RELATED ART

An organic light emitting device is one type of self-emissive display devices, and has advantages of having a wide viewing angle and a high response speed as well as having an excellent contrast.

The organic light emitting device has a structure of disposing an organic thin film between two electrodes. When a voltage is applied to the organic light emitting device having such a structure, electrons and holes injected from the two electrodes bind and pair in the organic thin film, and then light is emitted as these annihilate. The organic thin film may be formed in a single layer or a multilayer as necessary.

A material of the organic thin film may have a light emitting function as necessary. For example, as a material of the organic thin film, compounds each capable of forming a light emitting layer themselves alone may be used, or compounds each capable of serving as a host or a dopant of a host-dopant-based light emitting layer may also be used. In addition thereto, compounds capable of performing roles of hole injection, hole transport, electron blocking, hole blocking, electron transport, electron injection and the like may also be used as a material of the organic thin film.

Development of an organic thin film material has been continuously required for enhancing performance, lifetime or efficiency of an organic light emitting device.

### PRIOR ART DOCUMENTS

### Patent Documents

(Patent Document 1) U.S. Pat. No. 4,356,429

### SUMMARY

The present disclosure is directed to providing a heterocyclic compound, an organic light emitting device comprising the same, and a composition for an organic material layer.

One embodiment of the present disclosure provides a heterocyclic compound represented by the following Chemical Formula 1.

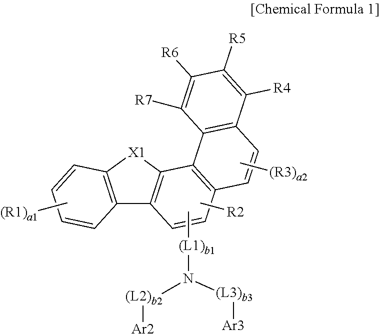

In Chemical Formula 1,
X1 is O; S; or NAr1,
R1 to R7 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other bond to each other to form a substituted or unsubstituted C6 to C60 aromatic hydrocarbon ring; or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
a1 is an integer of 0 to 4, and when a1 is 2 or greater, R1s are the same as or different from each other,
a2 is an integer of 0 to 2, and when a2 is 2 or greater, R3s are the same as or different from each other,
Ar1 is a substituted or unsubstituted C6 to C60 aryl group; or a substituted or unsubstituted C2 to C60 heteroaryl group,
Ar2 and Ar3 are the same as or different from each other, and each independently a substituted or unsubstituted C6 to C60 aryl group; a substituted or unsubstituted C2 to C60 heteroaryl group; or —NAr4Ar5,
Ar4 and Ar5 are the same as or different from each other, and each independently a substituted or unsubstituted C6 to C60 aryl group; or a substituted or unsubstituted C2 to C60 heteroaryl group,
L1 to L3 are the same as or different from each other, and each independently a direct bond; a substituted or unsubstituted C6 to C60 arylene group; or a substituted or unsubstituted C2 to C60 heteroarylene group,
b1 is an integer of 0 to 5, and when b1 is 2 or greater, L1s are the same as or different from each other,
b2 is an integer of 0 to 5, and when b2 is 2 or greater, L2s are the same as or different from each other, and
b3 is an integer of 0 to 5, and when b3 is 2 or greater, L3s are the same as or different from each other.

In addition, one embodiment of the present disclosure provides an organic light emitting device comprising:
a first electrode;
a second electrode provided opposite to the first electrode; and
one or more organic material layers provided between the first electrode and the second electrode,
wherein one or more layers of the one or more organic material layers comprise the heterocyclic compound represented by Chemical Formula 1.

In addition, one embodiment of the present disclosure provides an organic light emitting device, wherein the organic material layer comprises a hole transport layer, and the hole transport layer comprises the heterocyclic compound.

In addition, one embodiment of the present disclosure provides an organic light emitting device, wherein the organic material layer comprises an electron blocking layer, and the electron blocking layer comprises the heterocyclic compound.

In addition, one embodiment of the present disclosure provides an organic light emitting device, wherein the organic material layer comprises a light emitting layer, and the light emitting layer comprises the heterocyclic compound.

In addition, one embodiment of the present disclosure provides an organic light emitting device, wherein the organic material layer further comprises a heterocyclic compound represented by the following Chemical Formula 2.

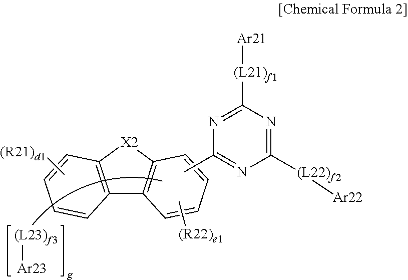

In Chemical Formula 2,
X2 is O; or S,
R21 and R22 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; —P(═O)R201R202; —SiR201R202R203; and —NR201R202, or two or more groups adjacent to each other bond to each other to form a substituted or unsubstituted C6 to C60 aromatic hydrocarbon ring; or a substituted or unsubstituted C2 to C60 heteroring, and R201, R202 and R203 are the same as or different from each other and each independently a substituted or unsubstituted C1 to C60 alkyl group; a substituted or unsubstituted C6 to C60 aryl group; or a substituted or unsubstituted C2 to C60 heteroaryl group,
d1 is an integer of 0 to 4, and when d1 is 2 or greater, R21s are the same as or different from each other,
e1 is an integer of 0 to 3, and when e1 is 2 or greater, R22s are the same as or different from each other,
L21 to L23 are the same as or different from each other, and each independently a direct bond; a substituted or unsubstituted C6 to C60 arylene group; or a substituted or unsubstituted C2 to C60 heteroarylene group,
f1 is an integer of 0 to 5, and when f1 is 2 or greater, L21s are the same as or different from each other,
f2 is an integer of 0 to 5, and when f2 is 2 or greater, L22s are the same as or different from each other,
f3 is an integer of 0 to 5, and when f3 is 2 or greater, L23s are the same as or different from each other,
Ar21 to Ar23 are the same as or different from each other, and each independently a substituted or unsubstituted C6 to C60 aryl group; a substituted or unsubstituted C2 to C60 heteroaryl group; or —NAr24Ar25,
Ar24 and Ar25 are the same as or different from each other, and each independently a substituted or unsubstituted C6 to C60 aryl group; or a substituted or unsubstituted C2 to C60 heteroaryl group, and
g is 0 or 1.

In addition, one embodiment of the present disclosure provides a composition for an organic material layer, the composition including: the heterocyclic compound represented by Chemical Formula 1; and the heterocyclic compound represented by Chemical Formula 2.

The compound described in the present specification can be used as a material of an organic material layer of an organic light emitting device. The compound is capable of playing roles of a hole injection layer material, an electron blocking layer material, a hole transport layer material, a light emitting layer material, an electron transport layer material, a hole blocking layer material, an electron injection layer material and the like in an organic light emitting device. Particularly, the compound can be used as a hole transport layer material, an electron blocking layer material or a light emitting layer material of an organic light emitting device.

Specifically, the compound represented by Chemical Formula 1 has fast hole mobility, and has appropriate HOMO (Highest Occupied Molecular Orbital) level and high LUMO (Lowest Unoccupied Molecular Orbital) level, and as a result, it is possible to lower a driving voltage, and improve light emission efficiency and lifetime properties.

### BRIEF DESCRIPTION OF THE DRAWINGS

FIGS. 1 to 3 are diagrams each schematically illustrating a lamination structure of an organic light emitting device according to one embodiment of the present disclosure.

### DESCRIPTION OF SPECIFIC EMBODIMENTS

Hereinafter, the present disclosure will be described in more detail.

In the present specification, a term “substitution” means that a hydrogen atom bonding to a carbon atom of a compound is changed to another substituent, and the position of substitution is not limited as long as it is a position at which the hydrogen atom is substituted, that is, a position at which a substituent is capable of substituting, and when two or more substituents substitute, the two or more substituents may be the same as or different from each other.

In the present specification, “substituted or unsubstituted” means being substituted with one or more substituents selected from the group consisting of deuterium; halogen; a cyano group; a C1 to C60 linear or branched alkyl group; a C2 to C60 linear or branched alkenyl group; a C2 to C60 linear or branched alkynyl group; a C1 to C60 linear, branched or cyclic alkoxy group; a C3 to C60 monocyclic or polycyclic cycloalkyl group; a C2 to C60 monocyclic or polycyclic heterocycloalkyl group; a C6 to C60 monocyclic or polycyclic aryl group; a C2 to C60 monocyclic or polycyclic heteroaryl group; —SiRR′R″; —P(═O)RR′; a C1 to C20 alkylamine group; a C6 to C60 monocyclic or polycyclic arylamine group; and a C2 to C60 monocyclic or polycyclic heteroarylamine group or being unsubstituted, or being substituted with a substituent in which two or more substituents selected from among the substituents exemplified above are linked or being unsubstituted, and R, R′ and R″ are the same as or different from each other and each independently a substituted or unsubstituted C1 to C60 alkyl group; a substituted or unsubstituted C6 to C60 aryl group; or a substituted or unsubstituted C2 to C60 heteroaryl group.

In the present specification, the halogen may be fluorine; chlorine; bromine; or iodine.

In the present specification, the alkyl group includes a linear or branched form having 1 to 60 carbon atoms, and may be further substituted with other substituents. The number of carbon atoms of the alkyl group may be from 1 to 60, specifically from 1 to 40 and more specifically from 1 to 20. Specific examples of the alkyl group may include a methyl group; an ethyl group; an n-propyl group; an isopropyl group; an n-butyl group; an isobutyl group; a tert-butyl group; a sec-butyl group; a 1-methyl-butyl group; a 1-ethyl-butyl group; an n-pentyl group; an isopentyl group; a neopentyl group; a tert-pentyl group; an n-hexyl group; a 1-methylpentyl group; a 2-methylpentyl group; a 4-methyl-2-pentyl group; a 3,3-dimethylbutyl group; a 2-ethylbutyl group; an n-heptyl group; a 1-methylhexyl group; a cyclopentylmethyl group; a cyclohexylmethyl group; an n-octyl group; a tert-octyl group; a 1-methylheptyl group; a 2-ethylhexyl group; a 2-propylpentyl group; an n-nonyl group; a 2,2-dimethylheptyl group; a 1-ethyl-propyl group; a 1,1-dimethyl-propyl group; an isohexyl group; a 4 methylhexyl group; a 5-methylhexyl group and the like, but are not limited thereto.

In the present specification, the alkenyl group includes a linear or branched form having 2 to 60 carbon atoms, and may be further substituted with other substituents. The number of carbon atoms of the alkenyl group may be from 2 to 60, specifically from 2 to 40 and more specifically from 2 to 20. Specific examples of the alkenyl group may include a vinyl group; a 1-propenyl group; an isopropenyl group; a 1-butenyl group; a 2-butenyl group; a 3-butenyl group; a 1-pentenyl group; a 2-pentenyl group; a 3-pentenyl group; a 3-methyl-1-butenyl group; a 1,3-butadienyl group; an allyl group; a 1-phenylvinyl-1-yl group; a 2-phenylvinyl-1-yl group; a 2,2-diphenylvinyl-1-yl group; a 2-phenyl-2-(naphthyl-1-yl) vinyl-1-yl group; a 2,2-bis(diphenyl-1-yl) vinyl-1-yl group; a stilbenyl group; a styrenyl group and the like, but are not limited thereto.

In the present specification, the alkynyl group includes a linear or branched form having 2 to 60 carbon atoms, and may be further substituted with other substituents. The number of carbon atoms of the alkynyl group may be from 2 to 60, specifically from 2 to 40 and more specifically from 2 to 20.

In the present specification, the alkoxy group may be linear, branched or cyclic. The number of carbon atoms of the alkoxy group is not particularly limited, but is preferably from 1 to 20. Specific examples of the alkoxy group may include a methoxy group; an ethoxy group; an n-propoxy group; an isopropoxy group; an n-butoxy group; an isobutoxy group; a tert-butoxy group; a sec-butoxy group; an n-pentyloxy group; a neopentyloxy group; an isopentyloxy group; an n-hexyloxy group; a 3,3-dimethylbutyloxy group; a 2-ethylbutyloxy group; an n-octyloxy group; an n-nonyloxy group; an n-decyloxy group; a benzyloxy group; a p-methylbenzyloxy group and the like, but are not limited thereto.

In the present specification, the cycloalkyl group includes a monocyclic or polycyclic group having 3 to 60 carbon atoms, and may be further substituted with other substituents. Herein, the polycyclic group means a group in which the cycloalkyl group is directly linked to or fused with another cyclic group. Herein, the another cyclic group may be a cycloalkyl group, but may also be different types of cyclic groups such as a heterocycloalkyl group; an aryl group; and a heteroaryl group. The number of carbon atoms of the cycloalkyl group may be from 3 to 60, specifically from 3 to 40 and more specifically from 5 to 20. Specific examples of the cycloalkyl group may include a cyclopropyl group; a cyclobutyl group; a cyclopentyl group; a 3-methylcyclopentyl group; a 2,3-dimethylcyclopentyl group; a cyclohexyl group; a 3-methylcyclohexyl group; a 4-methylcyclohexyl group; a 2,3-dimethylcyclohexyl group; a 3,4,5-trimethylcyclohexyl group; a 4-tert-butylcyclohexyl group; a cycloheptyl group; a cyclooctyl group and the like, but are not limited thereto.

In the present specification, the heterocycloalkyl group includes O; S; Se; N; or Si as a heteroatom, includes a monocyclic or polycyclic group having 2 to 60 carbon atoms, and may be further substituted with other substituents. Herein, the polycyclic group means a group in which the heterocycloalkyl group is directly linked to or fused with another cyclic group. Herein, the another cyclic group may be a heterocycloalkyl group, but may also be different types of cyclic groups such as a cycloalkyl group; an aryl group; and a heteroaryl group. The number of carbon atoms of the heterocycloalkyl group may be from 2 to 60, specifically from 2 to 40 and more specifically from 3 to 20.

In the present specification, the aryl group includes a monocyclic or polycyclic group having 6 to 60 carbon atoms, and may be further substituted with other substituents. Herein, the polycyclic group means a group in which the aryl group is directly linked to or fused with another cyclic group. Herein, the another cyclic group may be an aryl group, but may also be different types of cyclic groups such as a cycloalkyl group; a heterocycloalkyl group; and a heteroaryl group. The aryl group may include a spiro group. The number of carbon atoms of the aryl group may be from 6 to 60, specifically from 6 to 40 and more specifically from 6 to 20. Specific examples of the aryl group may include a phenyl group; a biphenyl group; a triphenyl group; a naphthyl group; an anthryl group; a chrysenyl group; a phenanthrenyl group; a perylenyl group; a fluoranthenyl group; a triphenylenyl group; a phenalenyl group; a pyrenyl group; a tetracenyl group; a pentacenyl group; a fluorenyl group; an indenyl group; an acenaphthylenyl group; a benzofluorenyl group; a spirobifluorenyl group; a 2,3-dihydro-1H-indenyl group; a fused ring group thereof, and the like, but are not limited thereto.

In the present specification, the phosphine oxide group is represented by —P(═O)R101R102, and R101 and R102 are the same as or different from each other and may be each independently a substituent formed with at least one of hydrogen; deuterium; a halogen group; an alkyl group; an alkenyl group; an alkoxy group; a cycloalkyl group; an aryl group; and a heterocyclic group. Specifically, the phosphine oxide group may be substituted with an aryl group, and as the aryl group, the examples described above may be applied. Examples of the phosphine oxide group may include a diphenylphosphine oxide group; a dinaphthylphosphine oxide group and the like, but are not limited thereto.

In the present specification, the silyl group is a substituent including Si and having the Si atom directly linked as a radical, and is represented by —SiR101R102R103. R101 to R103 are the same as or different from each other, and may be each independently a substituent formed with at least one of hydrogen; deuterium; a halogen group; an alkyl group; an alkenyl group; an alkoxy group; a cycloalkyl group; an aryl group; and a heterocyclic group. Specific examples of the silyl group may include a trimethylsilyl group; a triethylsilyl group; a t-butyldimethylsilyl group; a vinyldimethylsilyl group; a propyldimethylsilyl group; a triphenylsilyl group; a diphenylsilyl group; a phenylsilyl group and the like, but are not limited thereto.

In the present specification, the fluorenyl group may be substituted, and adjacent substituents may bond to each other to form a ring.

When the fluorenyl group is substituted,

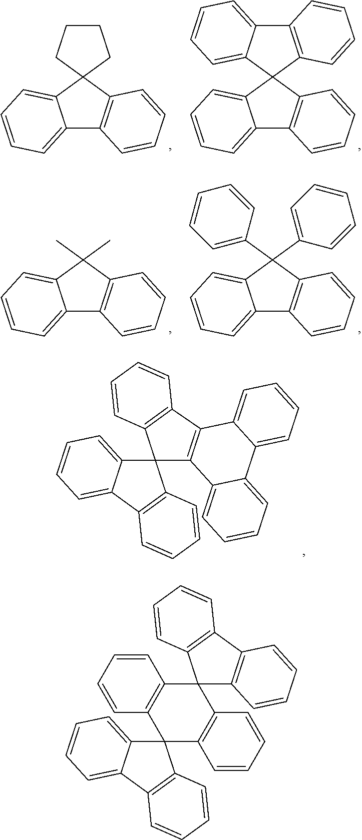

and the like may be included, however, the structure is not limited thereto.

In the present specification, the spiro group is a group including a spiro structure, and may have 15 to 60 carbon atoms. For example, the spiro group may include a structure in which a 2,3-dihydro-1H-indene group or a cyclohexane group spiro bonds to a fluorenyl group. Specifically, the spiro group may include any one of groups of the following structural formulae.

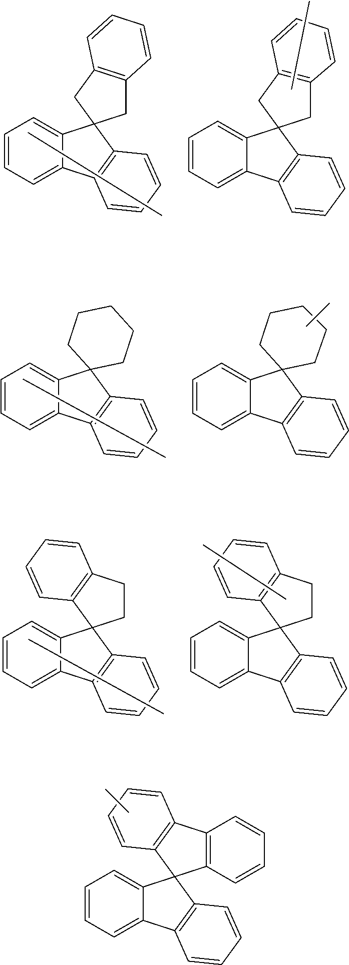

In the present specification, the heteroaryl group includes S, O, Se, N or Si as a heteroatom, includes a monocyclic or polycyclic group having 2 to 60 carbon atoms, and may be further substituted with other substituents. Herein, the polycyclic group means a group in which the heteroaryl group is directly linked to or fused with another cyclic group. Herein, the another cyclic group may be a heteroaryl group, but may also be different types of cyclic groups such as a cycloalkyl group; a heterocycloalkyl group; and an aryl group. The number of carbon atoms of the heteroaryl group may be from 2 to 60, specifically from 2 to 40 and more specifically from 3 to 25. Specific examples of the heteroaryl group may include a pyridyl group; a pyrrolyl group; a pyrimidyl group; a pyridazinyl group; a furanyl group; a thiophenyl group; an imidazolyl group; a pyrazolyl group; an oxazolyl group; an isoxazolyl group; a thiazolyl group; an isothiazolyl group; a triazolyl group; a furazanyl group; an oxadiazolyl group; a thiadiazolyl group; a dithiazolyl group; a tetrazolyl group; a pyranyl group; a thiopyranyl group; a diazinyl group; an oxazinyl group; a thiazinyl group; a dioxynyl group; a triazinyl group; a tetrazinyl group; a quinolyl group; an isoquinolyl group; a quinazolinyl group; an isoquinazolinyl group; a quinozolinyl group; a naphthyridyl group; an acridinyl group; a phenanthridinyl group; an imidazopyridinyl group; a diazanaphthalenyl group; a triazaindenyl group; a 2-indolyl group; an indolizinyl group; a benzothiazolyl group; a benzoxazolyl group; a benzimidazolyl group; a benzothiophenyl group; a benzofuranyl group; a dibenzothiophenyl group; a dibenzofuranyl group; a carbazolyl group; a benzocarbazolyl group; a dibenzocarbazolyl group; a phenazinyl group; a dibenzosilole group; a spirobi (dibenzosilole) group; a dihydrophenazinyl group; a phenoxazinyl group; a phenanthridyl group; a thienyl group; an indolo[2,3-a]carbazolyl group; an indolo[2,3-b]carbazolyl group; an indolinyl group; a 10,11-dihydrodibenzo[b,f]azepinyl group; a 9,10-dihydroacridinyl group; a phenanthrazinyl group; a phenothiazinyl group; a phthalazinyl group; a naphthylidinyl group; a phenanthrolinyl group; a benzo[c][1,2,5]thiadiazolyl group; a 5,10-dihydrodibenzo[b,c][1,4]azasilinyl group; a pyrazolo[1,5-c]quinazolinyl group; a pyrido[1,2-b]indazolyl group; a pyrido[1,2-a]imidazo[1,2-c]indolinyl group; a 5,11-dihydroindeno[1,2-b]carbazolyl group and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and the number of carbon atoms is not particularly limited, but preferably from 1 to 30. Specific examples of the amine group may include a methylamine group; a dimethylamine group; an ethylamine group; a diethylamine group; a phenylamine group; a naphthylamine group; a biphenylamine group; a dibiphenylamine group; an anthracenylamine group; a 9-methyl-anthracenylamine group; a diphenylamine group; a phenylnaphthylamine group; a ditolylamine group; a phenyltolylamine group; a triphenylamine group; a biphenylnaphthylamine group; a phenylbiphenylamine group; a biphenylfluorenylamine group; a phenyltriphenylenylamine group; a biphenyltriphenylenylamine group and the like, but are not limited thereto.

In the present specification, the arylene group means the aryl group having two bonding sites, that is, a divalent group. The descriptions on the aryl group provided above may be applied thereto except for those that are each a divalent group. In addition, the heteroarylene group means the heteroaryl group having two bonding sites, that is, a divalent group. The descriptions on the heteroaryl group provided above may be applied thereto except for those that are each a divalent group.

In the present specification, an “adjacent” group may mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting at ortho positions in a benzene ring, and two substituents substituting at the same carbon in an aliphatic ring may be interpreted as groups “adjacent” to each other.

In the present disclosure, a “case of a substituent being not indicated in a chemical formula or compound structure” means that a hydrogen atom bonds to a carbon atom. However, since deuterium (²H, D) is an isotope of hydrogen, some hydrogen atoms may be deuterium.

In one embodiment of the present disclosure, a “case of a substituent being not indicated in a chemical formula or compound structure” may mean that positions to which substituents may come are all hydrogen or deuterium. In other words, since deuterium is an isotope of hydrogen, some hydrogen atoms may be deuterium that is an isotope, and herein, a content of the deuterium may be from 0% to 100%.

In one embodiment of the present disclosure, in a “case of a substituent being not indicated in a chemical formula or compound structure”, hydrogen and deuterium may be used interchangeably in compounds when deuterium is not explicitly excluded such as “a deuterium content being 0%”, “a hydrogen content being 100%” or “substituents being all hydrogen”.

In one embodiment of the present disclosure, deuterium is one of isotopes of hydrogen, is an element having deuteron formed with one proton and one neutron as a nucleus, and may be expressed as hydrogen-2, and the elemental symbol thereof may also be written as D or ²H.

In one embodiment of the present disclosure, an isotope means an atom with the same atomic number (Z) but with a different mass number (A), and may also be interpreted as an element with the same number of protons but with a different number of neutrons.

In one embodiment of the present disclosure, a content T % of a specific substituent may be defined as T2/T1×100−T % when the total number of substituents that a basic compound may have is defined as T1, and the number of specific substituents among these is defined as T2.

In other words, in one example, having a deuterium content of 20% in a phenyl group represented by

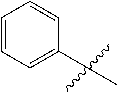 [SMILES: *c1ccccc1]

may mean that the total number of substituents that the phenyl group may have is 5 (T1 in the formula), and the number of deuterium atoms among these is 1 (T2 in the formula). In other words, having a deuterium content of 20% in a phenyl group may be represented by the following structural formulae.

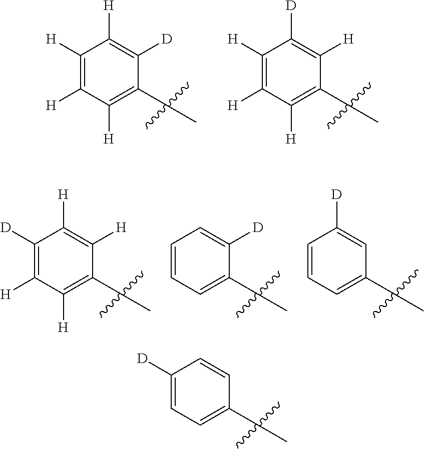 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, in one embodiment of the present disclosure, “a phenyl group having a deuterium content of 0%” may mean a phenyl group that does not include a deuterium atom, that is, a phenyl group that has 5 hydrogen atoms.

In the present disclosure, the C6 to C60 aromatic hydrocarbon ring means a compound including an aromatic ring formed with C6 to C60 carbons and hydrogens. Examples thereof may include phenyl, biphenyl, terphenyl, triphenylene, naphthalene, anthracene, phenalene, phenanthrene, fluorene, pyrene, chrysene, perylene, azulene and the like, but are not limited thereto, and include all aromatic hydrocarbon ring compounds known in the art satisfying the above-mentioned number of carbon atoms.

One embodiment of the present disclosure provides a heterocyclic compound represented by the following Chemical Formula 1.

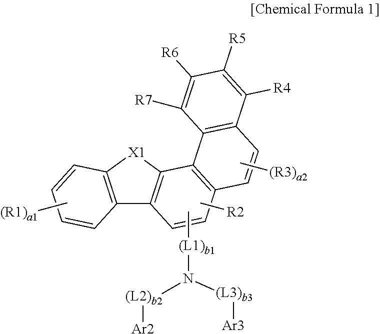

In Chemical Formula 1,
X1 is O; S; or NAr1,
R1 to R7 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other bond to each other to form a substituted or unsubstituted C6 to C60 aromatic hydrocarbon ring; or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
a1 is an integer of 0 to 4, and when a1 is 2 or greater, R1s are the same as or different from each other,
a2 is an integer of 0 to 2, and when a2 is 2 or greater, R3s are the same as or different from each other,
Ar1 is a substituted or unsubstituted C6 to C60 aryl group; or a substituted or unsubstituted C2 to C60 heteroaryl group,
Ar2 and Ar3 are the same as or different from each other, and each independently a substituted or unsubstituted C6 to C60 aryl group; a substituted or unsubstituted C2 to C60 heteroaryl group; or —NAr4Ar5,
Ar4 and Ar5 are the same as or different from each other, and each independently a substituted or unsubstituted C6 to C60 aryl group; or a substituted or unsubstituted C2 to C60 heteroaryl group,
L1 to L3 are the same as or different from each other, and each independently a direct bond; a substituted or unsubstituted C6 to C60 arylene group; or a substituted or unsubstituted C2 to C60 heteroarylene group,
b1 is an integer of 0 to 5, and when b1 is 2 or greater, L1s are the same as or different from each other,
b2 is an integer of 0 to 5, and when b2 is 2 or greater, L2s are the same as or different from each other, and
b3 is an integer of 0 to 5, and when b3 is 2 or greater, L3s are the same as or different from each other.

In one embodiment of the present disclosure, X1 may be O.

In another embodiment of the present disclosure, X1 may be S.

In another embodiment of the present disclosure, X1 may be NAr1.

In one embodiment of the present disclosure, Ar1 may be a substituted or unsubstituted C6 to C30 aryl group; or a substituted or unsubstituted C2 to C30 heteroaryl group.

In another embodiment of the present disclosure, Ar1 may be a substituted or unsubstituted C6 to C20 aryl group; or a substituted or unsubstituted C2 to C20 heteroaryl group.

In another embodiment of the present disclosure, Ar1 may be a substituted or unsubstituted C6 to C10 aryl group; or a substituted or unsubstituted C2 to C10 heteroaryl group.

In another embodiment of the present disclosure, Ar1 may be a substituted or unsubstituted C6 to C60 aryl group.

In another embodiment of the present disclosure, Ar1 may be a substituted or unsubstituted C6 5 to C30 aryl group.

In another embodiment of the present disclosure, Ar1 may be a substituted or unsubstituted C6 to C20 aryl group.

In another embodiment of the present disclosure, Ar1 may be a substituted or unsubstituted C6 to C10 aryl group.

In another embodiment of the present disclosure, Ar1 may be a substituted or unsubstituted phenyl group.

In one embodiment of the present disclosure, R1 to R7 are the same as or different from each other, and each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R101R102; —SiR101R102R103; or —NR101R102, or two or more groups adjacent to each other bond to each other to form a substituted or unsubstituted C6 to C30 aromatic hydrocarbon ring; or a substituted or unsubstituted C2 to C30 heteroring, and R101, R102 and R103 are the same as or different from each other and may be each independently a substituted or unsubstituted C1 to C30 alkyl group; a substituted or unsubstituted C6 to C30 aryl group; or a substituted or unsubstituted C2 to C30 heteroaryl group.

In another embodiment of the present disclosure, R1 to R7 are the same as or different from each other, and each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R101R102; —SiR101R102R103; or —NR101R102, or two or more groups adjacent to each other bond to each other to form a substituted or unsubstituted C6 to C20 aromatic hydrocarbon ring; or a substituted or unsubstituted C2 to C20 heteroring, and R101, R102 and R103 are the same as or different from each other and may be each independently a substituted or unsubstituted C1 to C20 alkyl group; a substituted or unsubstituted C6 to C20 aryl group; or a substituted or unsubstituted C2 to C20 heteroaryl group.

In another embodiment of the present disclosure, R1 to R7 are the same as or different from each other, and each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; or a substituted or unsubstituted C2 to C20 heteroaryl group, or two or more groups adjacent to each other bond to each other to form a substituted or unsubstituted C6 to C20 aromatic hydrocarbon ring; or a substituted or unsubstituted C2 to C20 heteroring.

In another embodiment of the present disclosure, R1 to R7 are the same as or different from each other, and each independently hydrogen; deuterium; halogen; a cyano group; a substituted or unsubstituted C1 to C20 alkyl group; a substituted or unsubstituted C6 to C20 aryl group; or a substituted or unsubstituted C2 to C20 heteroaryl group, or two or more groups adjacent to each other may bond to each other to form a substituted or unsubstituted C6 to C20 aromatic hydrocarbon ring; or a substituted or unsubstituted C2 to C20 heteroring.

In another embodiment of the present disclosure, R1 to R7 are the same as or different from each other, and each independently hydrogen; or deuterium, or two or more groups adjacent to each other may bond to each other to form a substituted or unsubstituted C6 to C20 aromatic hydrocarbon ring; or a substituted or unsubstituted C2 to C20 heteroring.

In another embodiment of the present disclosure, R1 to R3 are the same as or different from each other, and may be each independently hydrogen; or deuterium.

In another embodiment of the present disclosure, R4 to R7 are the same as or different from each other, and each independently hydrogen; or deuterium, or two or more groups adjacent to each other may bond to each other to form a substituted or unsubstituted C6 to C20 aromatic hydrocarbon ring; or a substituted or unsubstituted C2 to C20 heteroring.

In another embodiment of the present disclosure, R4 and R5 may bond to each other to form a substituted or unsubstituted C6 to C20 aromatic hydrocarbon ring as the two or more groups adjacent to each other, and R6 and R7 are the same as or different from each other and may be each independently hydrogen; or deuterium.

In another embodiment of the present disclosure, R5 and R6 may bond to each other to form a substituted or unsubstituted C6 to C20 aromatic hydrocarbon ring as the two or more groups adjacent to each other, and R4 and R7 are the same as or different from each other and may be each independently hydrogen; or deuterium.

In another embodiment of the present disclosure, R6 and R7 may bond to each other to form a substituted or unsubstituted C6 to C20 aromatic hydrocarbon ring as the two or more groups adjacent to each other, and R4 and R5 are the same as or different from each other and may be each independently hydrogen; or deuterium.

In one embodiment of the present disclosure, Ar2 and Ar3 are the same as or different from each other, and may be each independently a substituted or unsubstituted C6 to C30 aryl group; a substituted or unsubstituted C2 to C30 heteroaryl group; or —NAr4Ar5.

In another embodiment of the present disclosure, Ar2 and Ar3 are the same as or different from each other, and may be each independently a substituted or unsubstituted C6 to C20 aryl group; a substituted or unsubstituted C2 to C20 heteroaryl group; or —NAr4Ar5.

In another embodiment of the present disclosure, Ar2 and Ar3 are the same as or different from each other, and may be each independently a substituted or unsubstituted phenyl group; a substituted or unsubstituted biphenyl group; a substituted or unsubstituted terphenyl group; a substituted or unsubstituted naphthyl group; a substituted or unsubstituted phenanthrenyl group; a substituted or unsubstituted triphenylenyl group; a substituted or unsubstituted fluorenyl group; a substituted or unsubstituted spirobifluorenyl group; a substituted or unsubstituted dibenzofuranyl group; a substituted or unsubstituted dibenzothiophenyl group; a substituted or unsubstituted carbazolyl group; or —NAr4Ar5.

In one embodiment of the present disclosure, Ar4 and Ar5 are the same as or different from each other, and may be each independently a substituted or unsubstituted C6 to C30 aryl group; or a substituted or unsubstituted C2 to C30 heteroaryl group.

In another embodiment of the present disclosure, Ar4 and Ar5 are the same as or different from each other, and may be each independently a substituted or unsubstituted C6 to C20 aryl group; or a substituted or unsubstituted C2 to C20 heteroaryl group.

In another embodiment of the present disclosure, Ar4 and Ar5 are the same as or different from each other, and may be each independently a substituted or unsubstituted C6 to C10 aryl group; or a substituted or unsubstituted C2 to C10 heteroaryl group.

In another embodiment of the present disclosure, Ar4 and Ar5 are the same as or different from each other, and may be each independently a substituted or unsubstituted C6 to C60 aryl group.

In another embodiment of the present disclosure, Ar4 and Ar5 are the same as or different from each other, and may be each independently a substituted or unsubstituted C6 to C30 aryl group.

In another embodiment of the present disclosure, Ar4 and Ar5 are the same as or different from each other, and may be each independently a substituted or unsubstituted C6 to C20 aryl group.

In another embodiment of the present disclosure, Ar4 and Ar5 are the same as or different from each other, and may be each independently a substituted or unsubstituted C6 to C10 aryl group.

In another embodiment of the present disclosure, Ar4 and Ar5 may be a substituted or unsubstituted phenyl group.

In one embodiment of the present disclosure, L1 to L3 are the same as or different from each other, and may be each independently a direct bond; a substituted or unsubstituted C6 to C30 arylene group; or a substituted or unsubstituted C2 to C30 heteroarylene group.

In another embodiment of the present disclosure, L1 to L3 are the same as or different from each other, and may be each independently a direct bond; a substituted or unsubstituted C6 to C20 arylene group; or a substituted or unsubstituted C2 to C20 heteroarylene group.

In another embodiment of the present disclosure, L1 may be a direct bond; or a substituted or unsubstituted C6 to C60 arylene group.

In another embodiment of the present disclosure, L1 may be a direct bond; or a substituted or unsubstituted C6 to C30 arylene group.

In another embodiment of the present disclosure, L1 may be a direct bond; or a substituted or unsubstituted C6 to C20 arylene group.

In another embodiment of the present disclosure, L1 may be a direct bond; a substituted or unsubstituted phenylene group; or a substituted or unsubstituted biphenylene group.

In another embodiment of the present disclosure, L2 and L3 are the same as or different from each other, and may be each independently a direct bond; a substituted or unsubstituted C6 to C30 arylene group; or a substituted or unsubstituted C2 to C30 heteroarylene group.

In another embodiment of the present disclosure, L2 and L3 are the same as or different from each other, and may be each independently a direct bond; a substituted or unsubstituted C6 to C20 arylene group; or a substituted or unsubstituted C2 to C20 heteroarylene group.

In another embodiment of the present disclosure, L2 and L3 are the same as or different from each other, and may be each independently a direct bond; a substituted or unsubstituted phenylene group; a substituted or unsubstituted biphenylene group; or a substituted or unsubstituted carbazolene group.

In one embodiment of the present disclosure, the heterocyclic compound represented by Chemical Formula 1 may be represented by any one of the following Chemical Formulae 1-1 and 1-2.

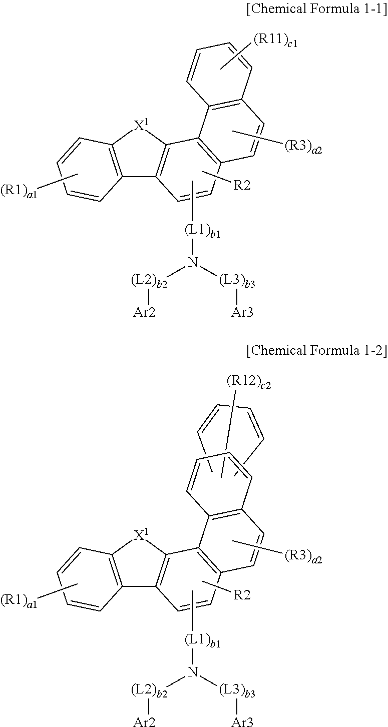

In Chemical Formulae 1-1 and 1-2,
R11 and R12 are the same as or different from each other and each independently hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; or —NR101R102, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group,
c1 is an integer of 0 to 4, and when c1 is 2 or greater, R11s are the same as or different from each other,
c2 is an integer of 0 to 6, and when c2 is 2 or greater, R12s are the same as or different from each other, and
X1, R1 to R3, L1 to L3, Ar2, Ar3, a1, a2, and b1 to b3 have the same definitions as in Chemical Formula 1.

In one embodiment of the present disclosure, R11 and R12 are the same as or different from each other and may be each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; a substituted or unsubstituted C6 to C30 aryl group; a substituted or unsubstituted C2 to C30 heteroaryl group; —P(═O)R101R102; —SiR101R102R103; or —NR101R102, and R101, R102 and R103 are the same as or different from each other and may be each independently a substituted or unsubstituted C1 to C30 alkyl group; a substituted or unsubstituted C6 to C30 aryl group; or a substituted or unsubstituted C2 to C30 heteroaryl group.

In another embodiment of the present disclosure, R11 and R12 are the same as or different from each other and may be each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; a substituted or unsubstituted C2 to C20 heteroaryl group; —P(═O)R101R102; —SiR101R102R103; or —NR101R102, and R101, R102 and R103 are the same as or different from each other and may be each independently a substituted or unsubstituted C1 to C20 alkyl group; a substituted or unsubstituted C6 to C20 aryl group; or a substituted or unsubstituted C2 to C20 heteroaryl group.

In another embodiment of the present disclosure, R11 and R12 are the same as or different from each other, and may be each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; a substituted or unsubstituted C6 to C20 aryl group; or a substituted or unsubstituted C2 to C20 heteroaryl group.

In another embodiment of the present disclosure, R11 and R12 are the same as or different from each other, and may be each independently hydrogen; deuterium; a substituted or unsubstituted C1 to C20 alkyl group; a substituted or unsubstituted C6 to C20 aryl group; or a substituted or unsubstituted C2 to C20 heteroaryl group.

In another embodiment of the present disclosure, R11 and R12 are the same as or different from each other, and may be each independently hydrogen; or deuterium.

In one embodiment of the present disclosure, the heterocyclic compound represented by Chemical Formula 1-1 may be represented by the following Chemical Formula 1-1a or 1-1b.

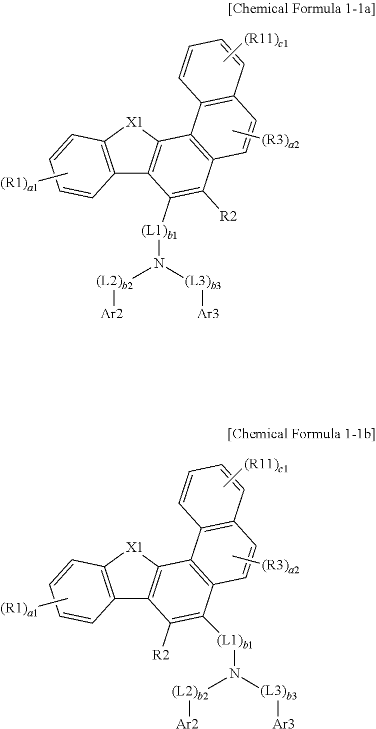

In Chemical Formulae 1-1a and 1-1b,
R11 and b1 have the same definitions as in Chemical Formula 1-1, and
X1, R1 to R3, L1 to L3, Ar2, Ar3, a1, a2, and b1 to b3 have the same definitions as in Chemical Formula 1.

In one embodiment of the present disclosure, the heterocyclic compound represented by Chemical Formula 1-2 may be represented by any one of the following Chemical Formulae 1-2-1 to 1-2-3.

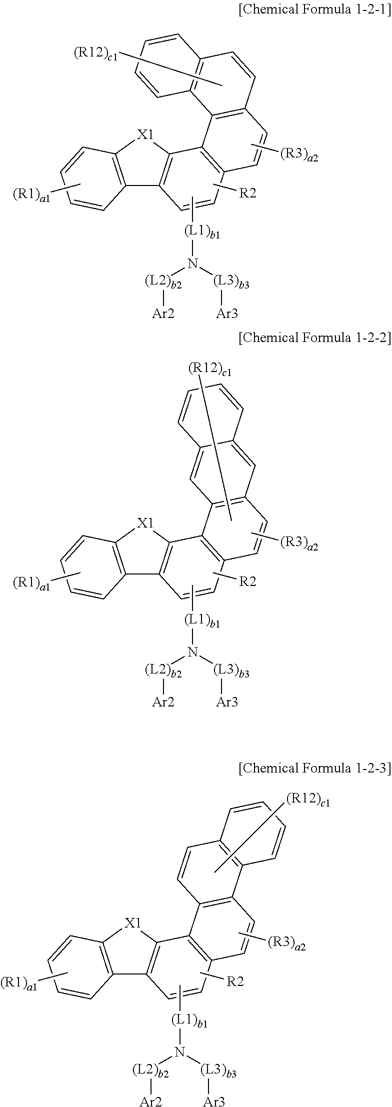

In Chemical Formulae 1-2-1 to 1-2-3,
R12 and c2 have the same definitions as in Chemical Formula 1-2, and
X1, R1 to R3, L1 to L3, Ar2, Ar3, a1, a2, and b1 to b3 have the same definitions as in Chemical Formula 1.

In one embodiment of the present disclosure, the heterocyclic compound represented by Chemical Formula 1-2-1 may be represented by the following Chemical Formula 1-2-1a or 1-2-1b.

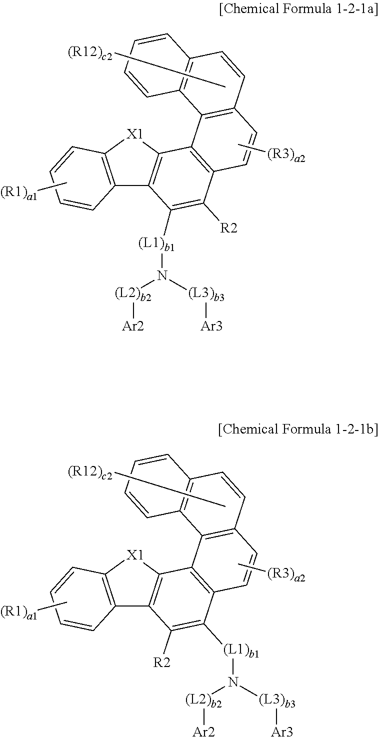

In Chemical Formulae 1-2-1a and 1-2-1b,
R12 and c2 have the same definitions as in Chemical Formula 1-2, and
X1, R1 to R3, L1 to L3, Ar2, Ar3, a1, a2, and b1 to b3 have the same definitions as in Chemical Formula 1.

In one embodiment of the present disclosure, the heterocyclic compound represented by Chemical Formula 1-2-2 may be represented by the following Chemical Formula 1-2-2a or 1-2-2b.

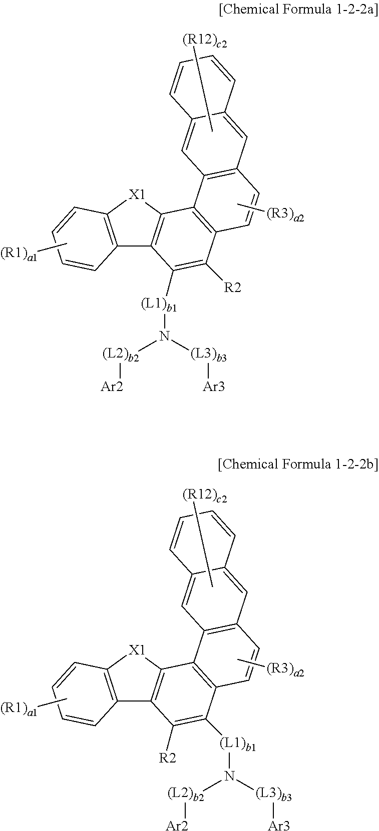

In Chemical Formulae 1-2-2a and 1-2-2b,
R12 and c2 have the same definitions as in Chemical Formula 1-2, and
X1, R1 to R3, L1 to L3, Ar2, Ar3, a1, a2, and b1 to b3 have the same definitions as in Chemical Formula 1.

In one embodiment of the present disclosure, the heterocyclic compound represented by Chemical Formula 1-2-3 may be represented by the following Chemical Formula 1-2-3a or 1-2-3b.

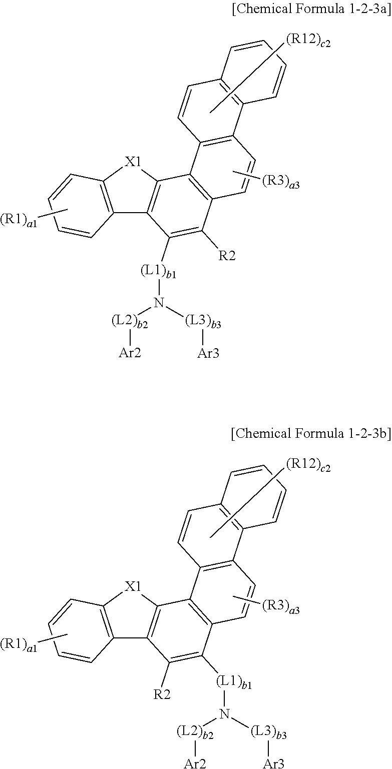

In Chemical Formulae 1-2-3a and 1-2-3b,
R12 and c2 have the same definitions as in Chemical Formula 1-2, and
X1, R1 to R3, L1 to L3, Ar2, Ar3, a1, a2, and b1 to b3 have the same definitions as in Chemical Formula 1.

In one embodiment of the present disclosure, R1 to R7, R11, R12, L1 to L3, and Ar1 to Ar5 may all include hydrogen (H) that is not deuterated.

In another embodiment of the present disclosure, at least one of R1 to R7, R11, R12, L1 to L3, and Ar1 to Ar5 includes deuterium (D), and at least one of R1 to R7, R11, R12, L1 to L3, and Ar1 to Ar5 may include hydrogen that is not deuterated.

In another embodiment of the present disclosure, R1 to R7, R11, R12, L1 to L3, and Ar1 to Ar5 may all include deuterium.

In one embodiment of the present disclosure, the heterocyclic compound represented by Chemical Formula 1 may not include deuterium as a substituent, or may have a deuterium content of, for example, greater than 0%, 1% or greater, 10% or greater, 20% or greater, 30% or greater, 40% or greater or 50% or greater, and 100% or less, 90% or less, 80% or less, 70% or less or 60% or less with respect to the total number of hydrogen atoms and deuterium atoms.

In another embodiment of the present disclosure, the heterocyclic compound represented by Chemical Formula 1 may not include deuterium as a substituent, or may have a deuterium content of 1% to 100% with respect to the total number of hydrogen atoms and deuterium atoms.

In another embodiment of the present disclosure, the heterocyclic compound represented by Chemical Formula 1 may not include deuterium as a substituent, or may have a deuterium content of 20% to 90% with respect to the total number of hydrogen atoms and deuterium atoms.

In another embodiment of the present disclosure, the heterocyclic compound represented by Chemical Formula 1 may not include deuterium as a substituent, or may have a deuterium content of 30% to 80% with respect to the total number of hydrogen atoms and deuterium atoms.

In another embodiment of the present disclosure, the heterocyclic compound represented by Chemical Formula 1 may not include deuterium as a substituent, or may have a deuterium content of 50% to 70% with respect to the total number of hydrogen atoms and deuterium atoms.

In one embodiment of the present disclosure, the heterocyclic compound represented by Chemical Formula 1 may be represented by any one of the following compounds.

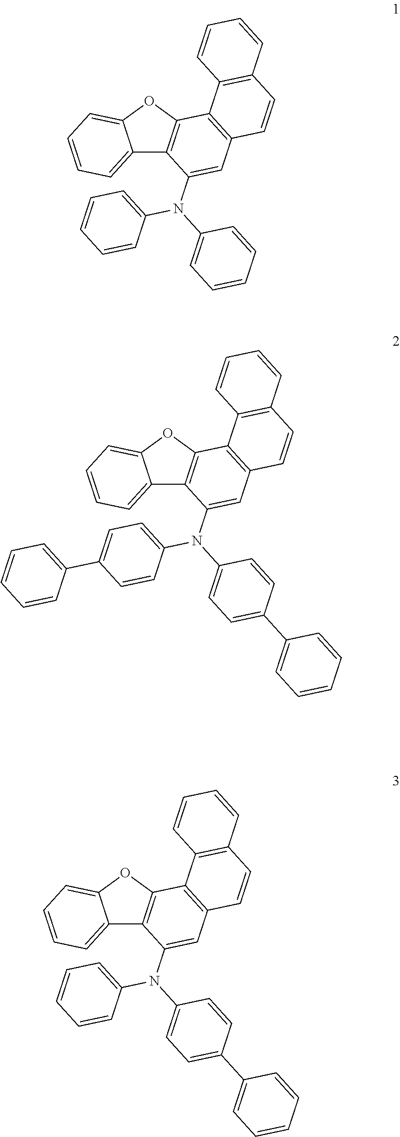 [SMILES: c1ccc(N(c2ccccc2)c2cc3ccc4ccccc4c3c3oc4ccccc4c23)cc1 ; c1ccc(-c2ccc(N(c3ccc(-c4ccccc4)cc3)c3cc4ccc5ccccc5c4c4oc5ccccc5c34)cc2)cc1 ; c1ccc(-c2ccc(N(c3ccccc3)c3cc4ccc5ccccc5c4c4oc5ccccc5c34)cc2)cc1]

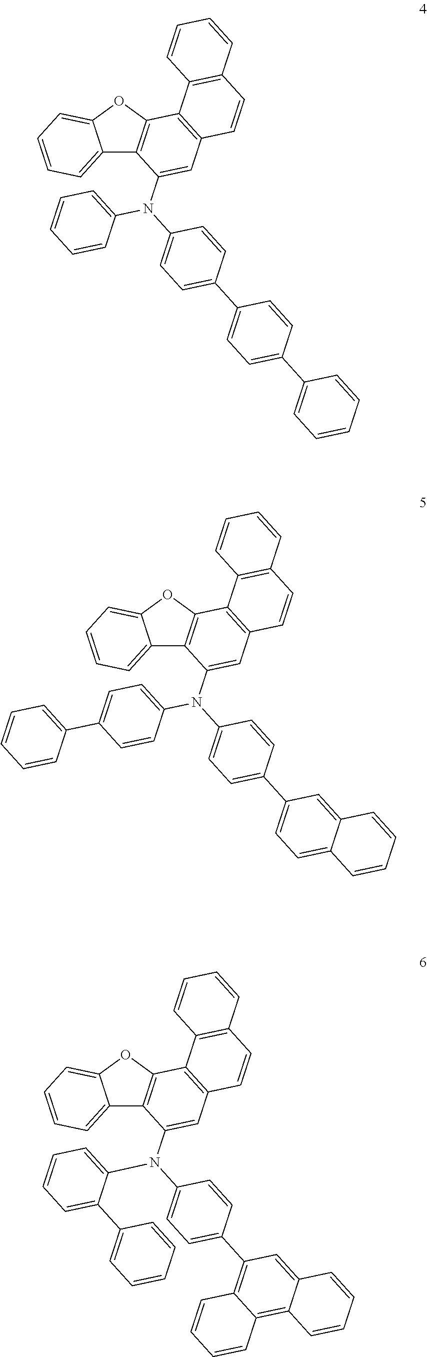 [SMILES: c1ccc(-c2ccc(-c3ccc(N(c4ccccc4)c4cc5ccc6ccccc6c5c5oc6ccccc6c45)cc3)cc2)cc1 ; c1ccc(-c2ccc(N(c3ccc(-c4ccc5ccccc5c4)cc3)c3cc4ccc5ccccc5c4c4oc5ccccc5c34)cc2)cc1 ; c1ccc(-c2ccccc2N(c2ccc(-c3cc4ccccc4c4ccccc34)cc2)c2cc3ccc4ccccc4c3c3oc4ccccc4c23)cc1]

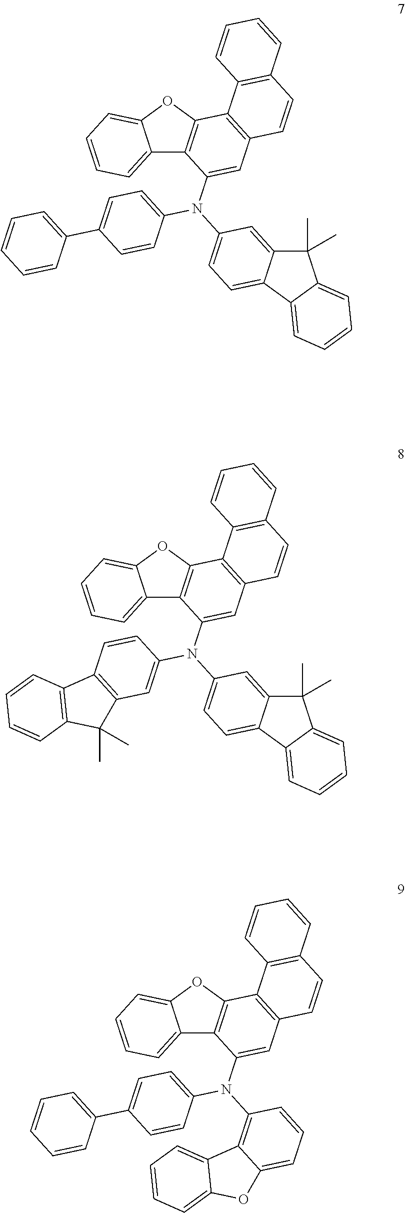 [SMILES: CC1(C)c2ccccc2-c2ccc(N(c3ccc(-c4ccccc4)cc3)c3cc4ccc5ccccc5c4c4oc5ccccc5c34)cc21 ; CC1(C)c2ccccc2-c2ccc(N(c3ccc4c(c3)C(C)(C)c3ccccc3-4)c3cc4ccc5ccccc5c4c4oc5ccccc5c34)cc21 ; c1ccc(-c2ccc(N(c3cccc4oc5ccccc5c34)c3cc4ccc5ccccc5c4c4oc5ccccc5c34)cc2)cc1]

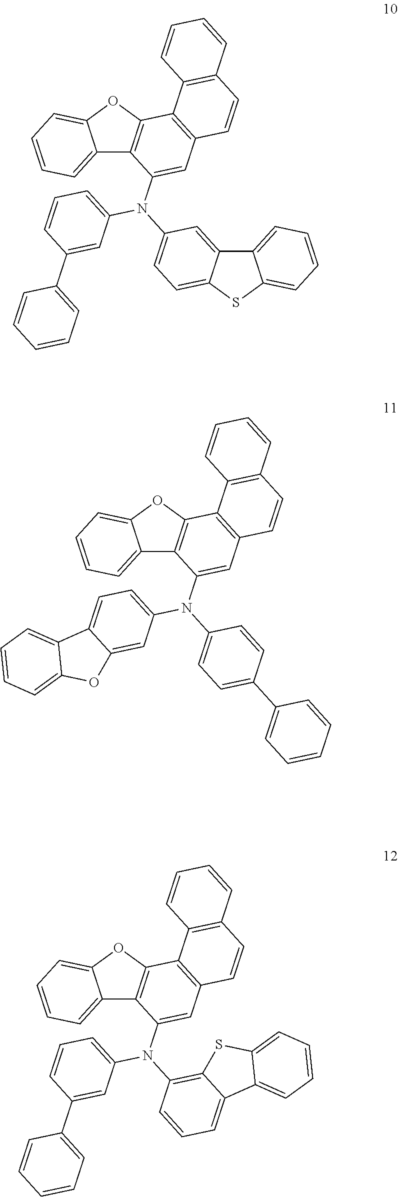 [SMILES: c1ccc(-c2cccc(N(c3ccc4sc5ccccc5c4c3)c3cc4ccc5ccccc5c4c4oc5ccccc5c34)c2)cc1 ; c1ccc(-c2ccc(N(c3ccc4c(c3)oc3ccccc34)c3cc4ccc5ccccc5c4c4oc5ccccc5c34)cc2)cc1 ; c1ccc(-c2cccc(N(c3cccc4c3sc3ccccc34)c3cc4ccc5ccccc5c4c4oc5ccccc5c34)c2)cc1]

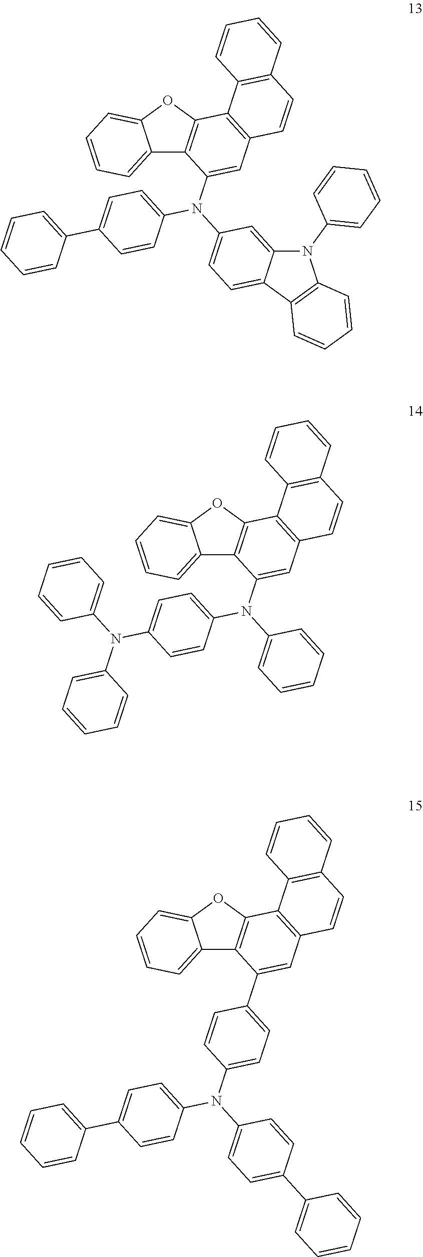 [SMILES: c1ccc(-c2ccc(N(c3ccc4c5ccccc5n(-c5ccccc5)c4c3)c3cc4ccc5ccccc5c4c4oc5ccccc5c34)cc2)cc1 ; c1ccc(N(c2ccccc2)c2ccc(N(c3ccccc3)c3cc4ccc5ccccc5c4c4oc5ccccc5c34)cc2)cc1 ; c1ccc(-c2ccc(N(c3ccc(-c4ccccc4)cc3)c3ccc(-c4cc5ccc6ccccc6c5c5oc6ccccc6c45)cc3)cc2)cc1]

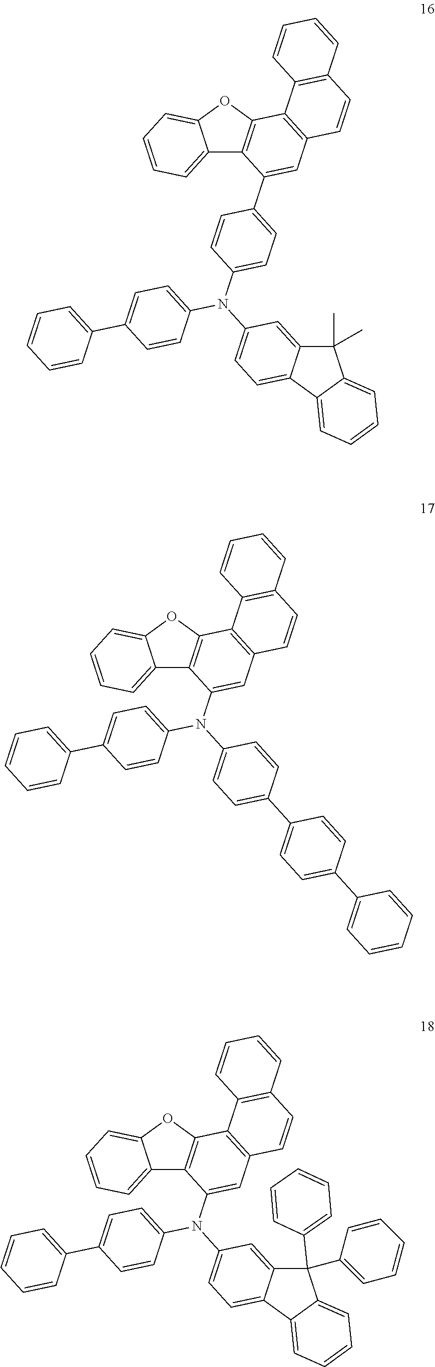 [SMILES: CC1(C)c2ccccc2-c2ccc(N(c3ccc(-c4ccccc4)cc3)c3ccc(-c4cc5ccc6ccccc6c5c5oc6ccccc6c45)cc3)cc21 ; c1ccc(-c2ccc(-c3ccc(N(c4ccc(-c5ccccc5)cc4)c4cc5ccc6ccccc6c5c5oc6ccccc6c45)cc3)cc2)cc1 ; c1ccc(-c2ccc(N(c3ccc4c(c3)C(c3ccccc3)(c3ccccc3)c3ccccc3-4)c3cc4ccc5ccccc5c4c4oc5ccccc5c34)cc2)cc1]

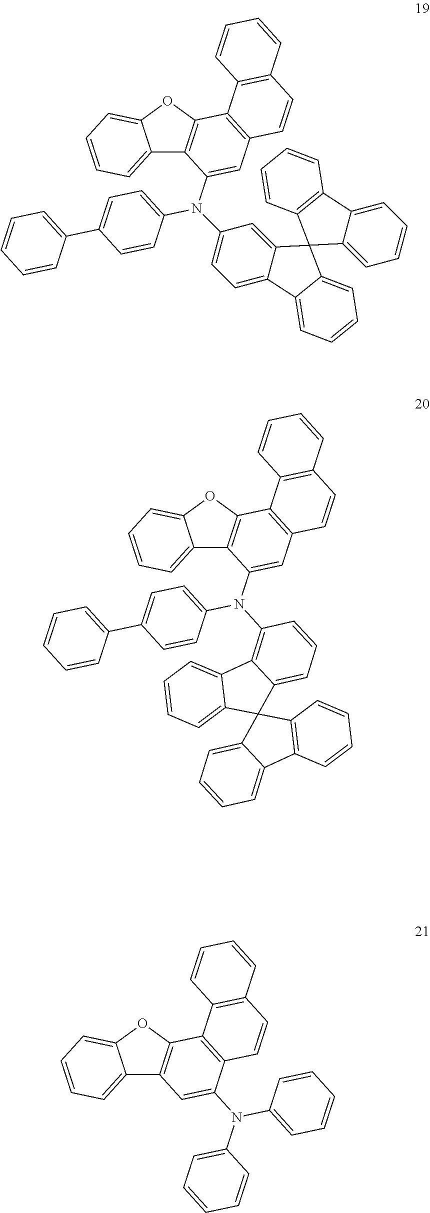 [SMILES: c1ccc(-c2ccc(N(c3ccc4c(c3)C3(c5ccccc5-c5ccccc53)c3ccccc3-4)c3cc4ccc5ccccc5c4c4oc5ccccc5c34)cc2)cc1 ; c1ccc(-c2ccc(N(c3cccc4c3-c3ccccc3C43c4ccccc4-c4ccccc43)c3cc4ccc5ccccc5c4c4oc5ccccc5c34)cc2)cc1 ; c1ccc(N(c2ccccc2)c2cc3c4ccccc4oc3c3c2ccc2ccccc23)cc1]

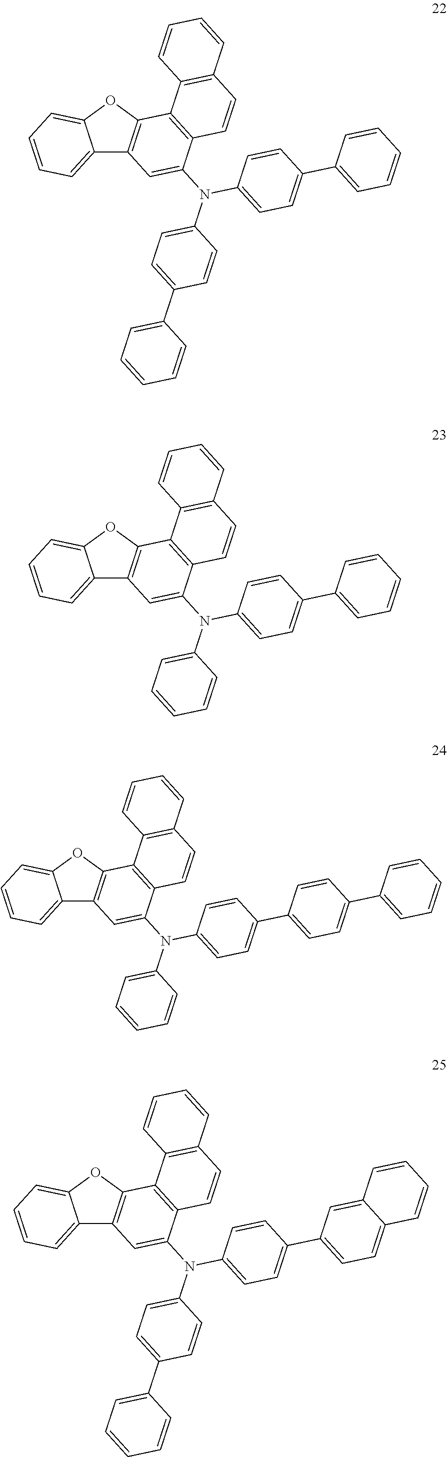 [SMILES: c1ccc(-c2ccc(N(c3ccc(-c4ccccc4)cc3)c3cc4c5ccccc5oc4c4c3ccc3ccccc34)cc2)cc1 ; c1ccc(-c2ccc(N(c3ccccc3)c3cc4c5ccccc5oc4c4c3ccc3ccccc34)cc2)cc1 ; c1ccc(-c2ccc(-c3ccc(N(c4ccccc4)c4cc5c6ccccc6oc5c5c4ccc4ccccc45)cc3)cc2)cc1 ; c1ccc(-c2ccc(N(c3ccc(-c4ccc5ccccc5c4)cc3)c3cc4c5ccccc5oc4c4c3ccc3ccccc34)cc2)cc1]

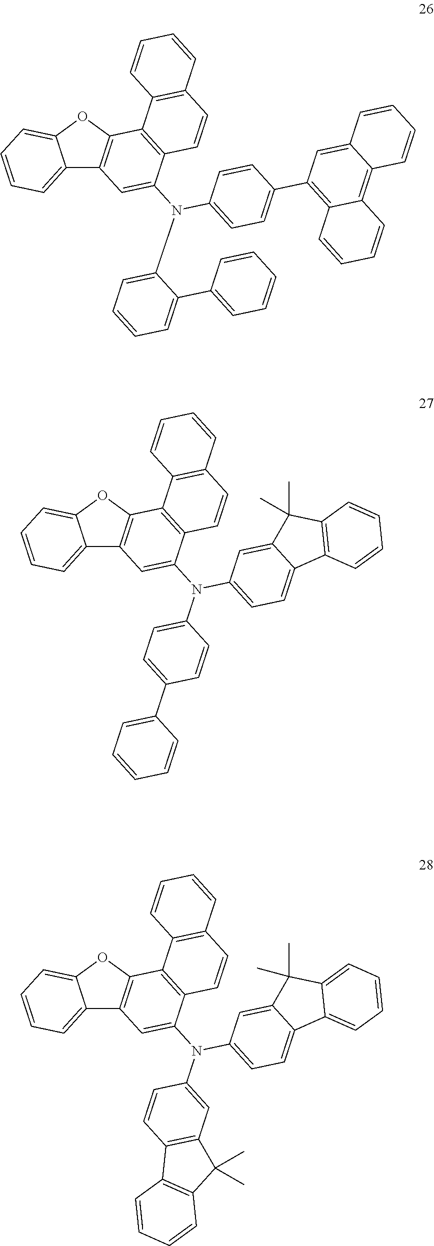 [SMILES: c1ccc(-c2ccccc2N(c2ccc(-c3cc4ccccc4c4ccccc34)cc2)c2cc3c4ccccc4oc3c3c2ccc2ccccc23)cc1 ; CC1(C)c2ccccc2-c2ccc(N(c3ccc(-c4ccccc4)cc3)c3cc4c5ccccc5oc4c4c3ccc3ccccc34)cc21 ; CC1(C)c2ccccc2-c2ccc(N(c3ccc4c(c3)C(C)(C)c3ccccc3-4)c3cc4c5ccccc5oc4c4c3ccc3ccccc34)cc21]

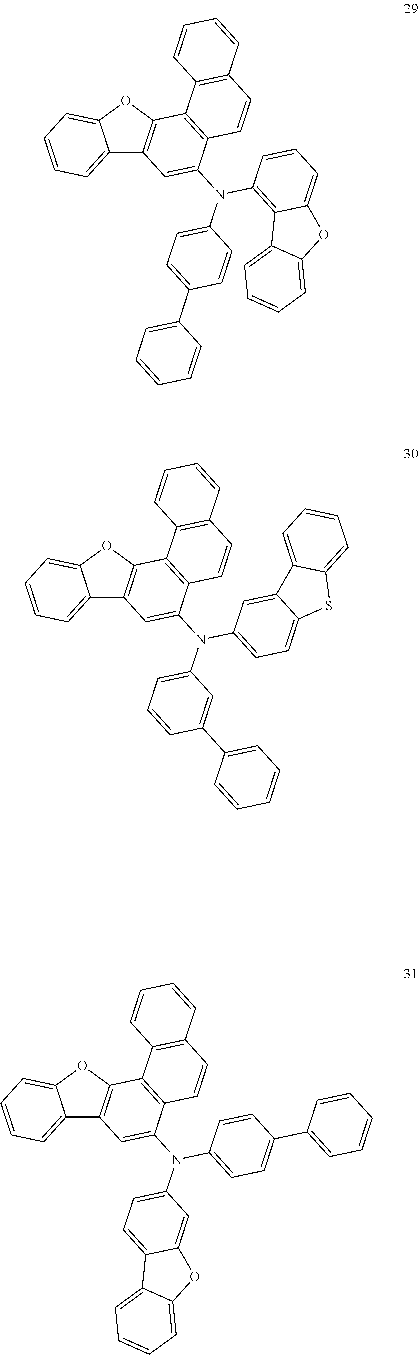 [SMILES: c1ccc(-c2ccc(N(c3cc4c5ccccc5oc4c4c3ccc3ccccc34)c3cccc4oc5ccccc5c34)cc2)cc1 ; c1ccc(-c2cccc(N(c3ccc4sc5ccccc5c4c3)c3cc4c5ccccc5oc4c4c3ccc3ccccc34)c2)cc1 ; c1ccc(-c2ccc(N(c3ccc4c(c3)oc3ccccc34)c3cc4c5ccccc5oc4c4c3ccc3ccccc34)cc2)cc1]

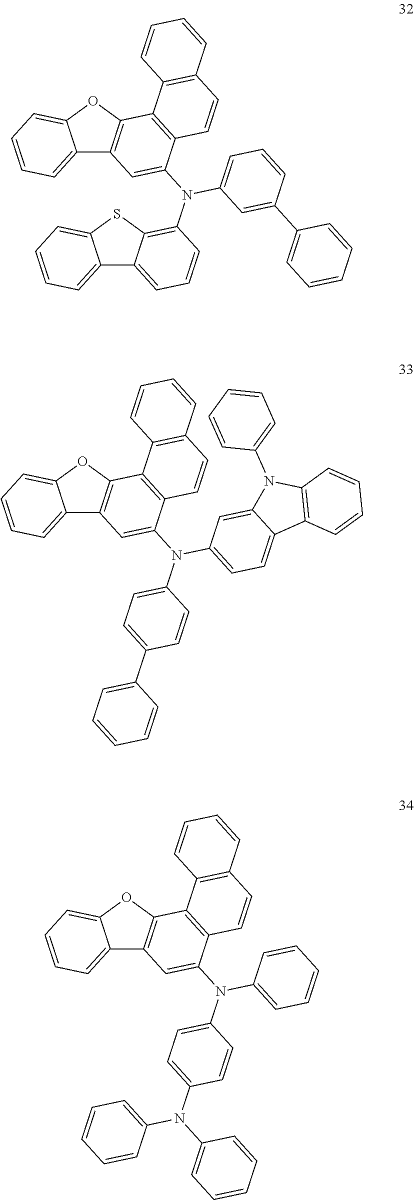 [SMILES: c1ccc(-c2cccc(N(c3cc4c5ccccc5oc4c4c3ccc3ccccc34)c3cccc4c3sc3ccccc34)c2)cc1 ; c1ccc(-c2ccc(N(c3ccc4c5ccccc5n(-c5ccccc5)c4c3)c3cc4c5ccccc5oc4c4c3ccc3ccccc34)cc2)cc1 ; c1ccc(N(c2ccccc2)c2ccc(N(c3ccccc3)c3cc4c5ccccc5oc4c4c3ccc3ccccc34)cc2)cc1]

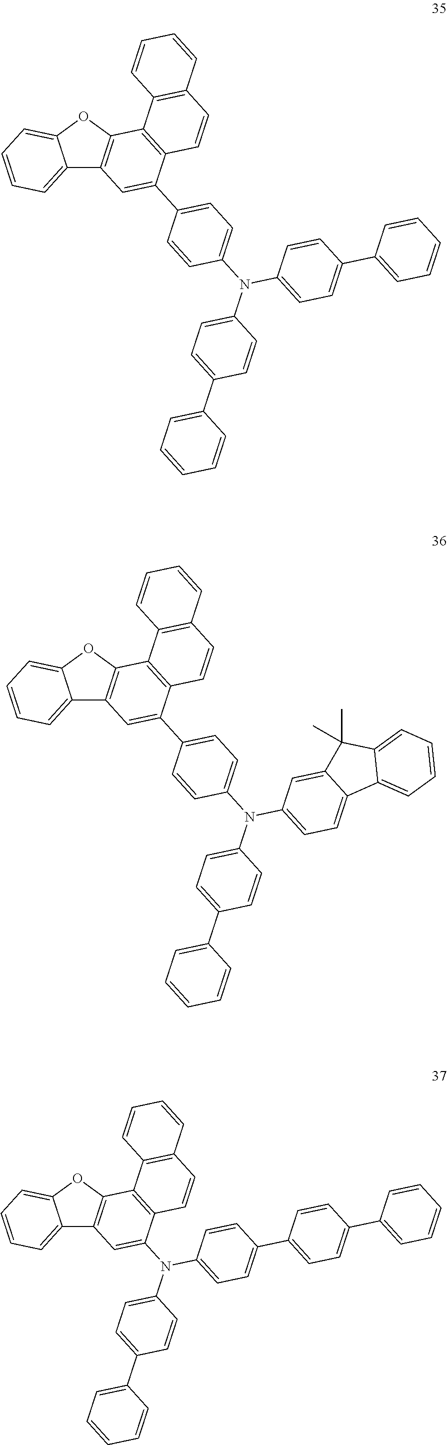 [SMILES: c1ccc(-c2ccc(N(c3ccc(-c4ccccc4)cc3)c3ccc(-c4cc5c6ccccc6oc5c5c4ccc4ccccc45)cc3)cc2)cc1 ; CC1(C)c2ccccc2-c2ccc(N(c3ccc(-c4ccccc4)cc3)c3ccc(-c4cc5c6ccccc6oc5c5c4ccc4ccccc45)cc3)cc21 ; c1ccc(-c2ccc(-c3ccc(N(c4ccc(-c5ccccc5)cc4)c4cc5c6ccccc6oc5c5c4ccc4ccccc45)cc3)cc2)cc1]

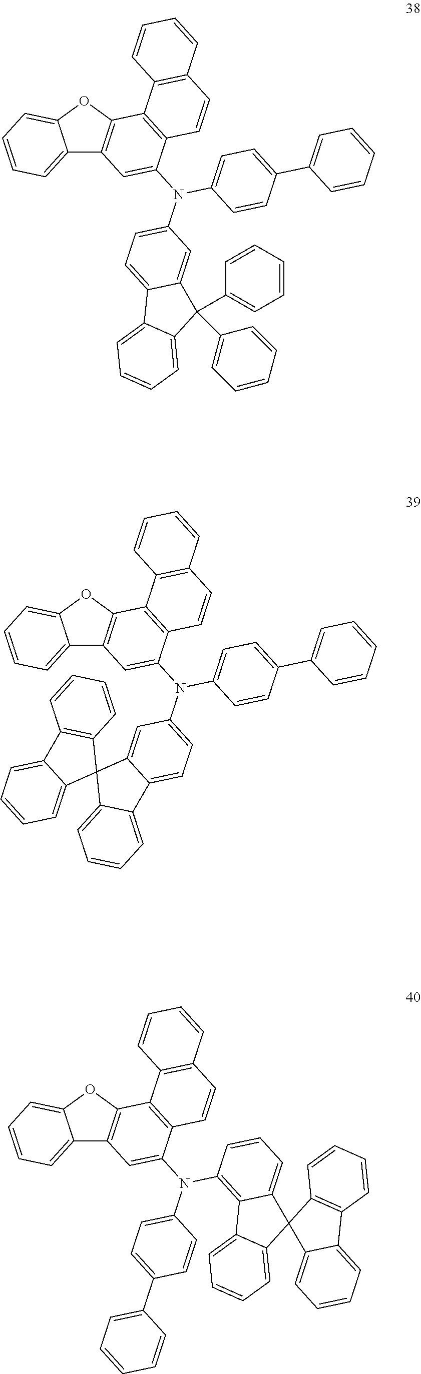 [SMILES: c1ccc(-c2ccc(N(c3ccc4c(c3)C(c3ccccc3)(c3ccccc3)c3ccccc3-4)c3cc4c5ccccc5oc4c4c3ccc3ccccc34)cc2)cc1 ; c1ccc(-c2ccc(N(c3ccc4c(c3)C3(c5ccccc5-c5ccccc53)c3ccccc3-4)c3cc4c5ccccc5oc4c4c3ccc3ccccc34)cc2)cc1 ; c1ccc(-c2ccc(N(c3cccc4c3-c3ccccc3C43c4ccccc4-c4ccccc43)c3cc4c5ccccc5oc4c4c3ccc3ccccc34)cc2)cc1]

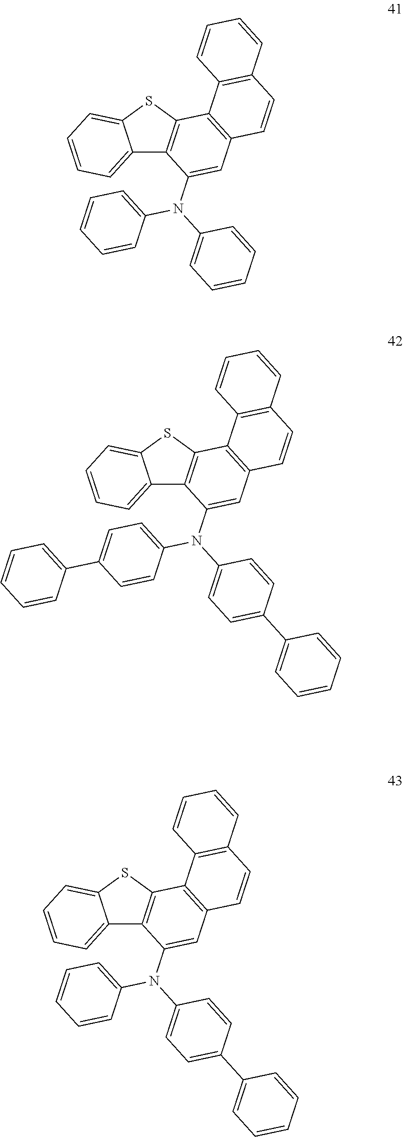 [SMILES: c1ccc(N(c2ccccc2)c2cc3ccc4ccccc4c3c3sc4ccccc4c23)cc1 ; c1ccc(-c2ccc(N(c3ccc(-c4ccccc4)cc3)c3cc4ccc5ccccc5c4c4sc5ccccc5c34)cc2)cc1 ; c1ccc(-c2ccc(N(c3ccccc3)c3cc4ccc5ccccc5c4c4sc5ccccc5c34)cc2)cc1]

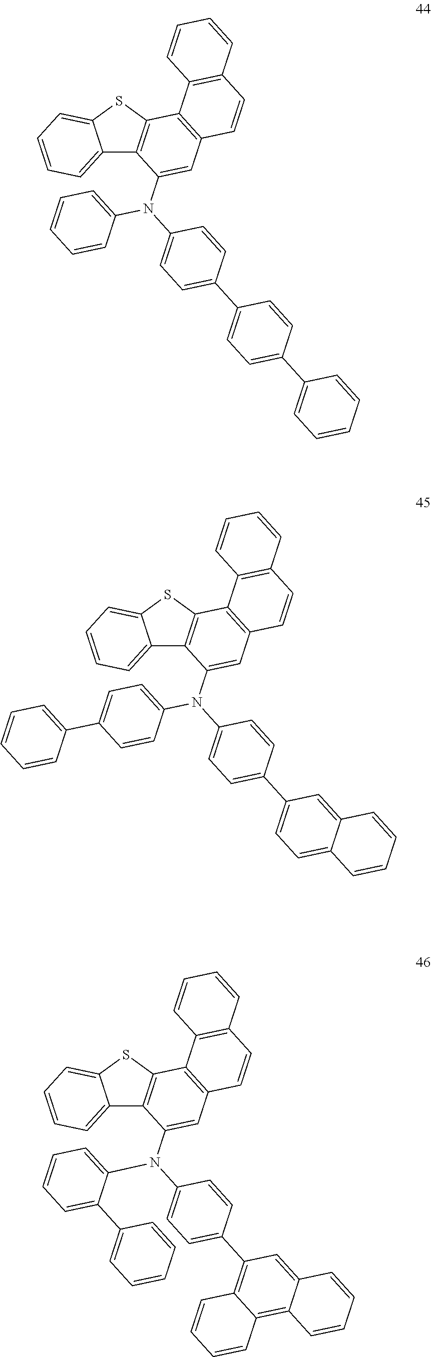 [SMILES: c1ccc(-c2ccc(-c3ccc(N(c4ccccc4)c4cc5ccc6ccccc6c5c5sc6ccccc6c45)cc3)cc2)cc1 ; c1ccc(-c2ccc(N(c3ccc(-c4ccc5ccccc5c4)cc3)c3cc4ccc5ccccc5c4c4sc5ccccc5c34)cc2)cc1 ; c1ccc(-c2ccccc2N(c2ccc(-c3cc4ccccc4c4ccccc34)cc2)c2cc3ccc4ccccc4c3c3sc4ccccc4c23)cc1]

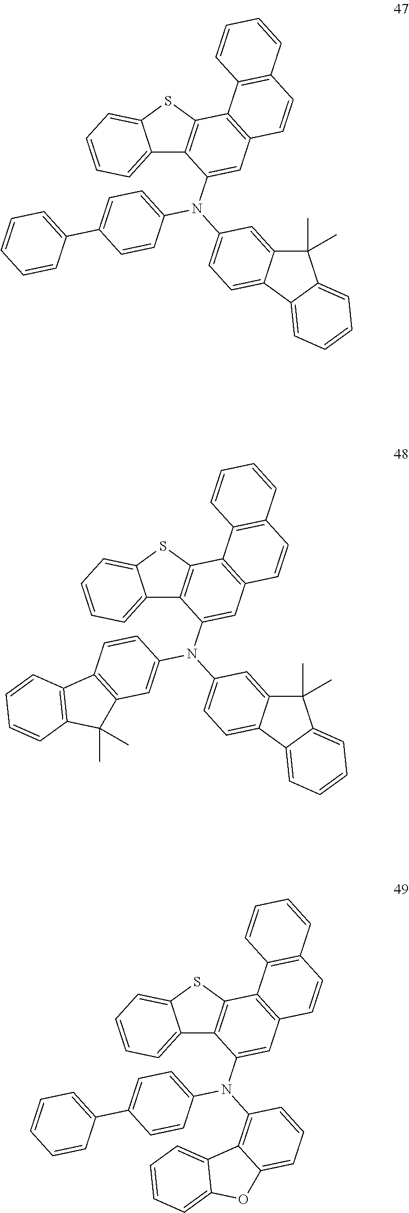 [SMILES: CC1(C)c2ccccc2-c2ccc(N(c3ccc(-c4ccccc4)cc3)c3cc4ccc5ccccc5c4c4sc5ccccc5c34)cc21 ; CC1(C)c2ccccc2-c2ccc(N(c3ccc4c(c3)C(C)(C)c3ccccc3-4)c3cc4ccc5ccccc5c4c4sc5ccccc5c34)cc21 ; c1ccc(-c2ccc(N(c3cccc4oc5ccccc5c34)c3cc4ccc5ccccc5c4c4sc5ccccc5c34)cc2)cc1]

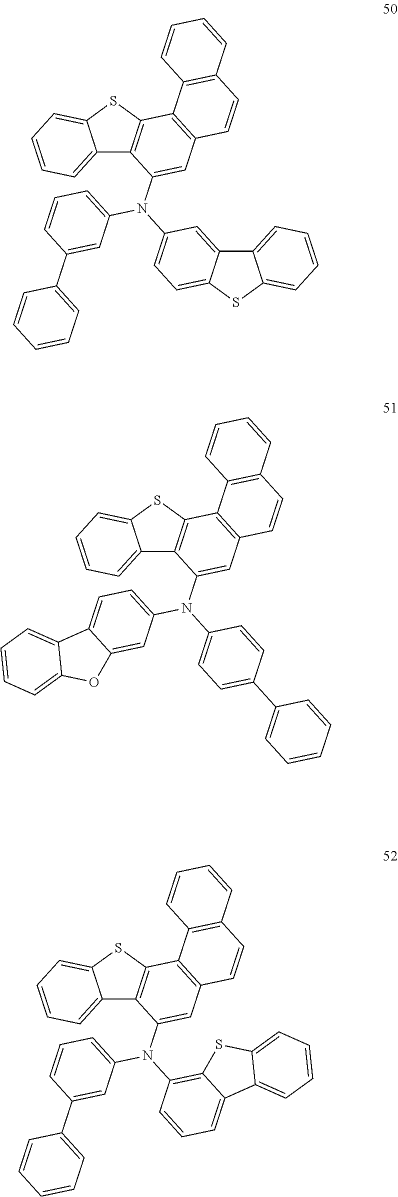 [SMILES: c1ccc(-c2cccc(N(c3ccc4sc5ccccc5c4c3)c3cc4ccc5ccccc5c4c4sc5ccccc5c34)c2)cc1 ; c1ccc(-c2ccc(N(c3ccc4c(c3)oc3ccccc34)c3cc4ccc5ccccc5c4c4sc5ccccc5c34)cc2)cc1 ; c1ccc(-c2cccc(N(c3cccc4c3sc3ccccc34)c3cc4ccc5ccccc5c4c4sc5ccccc5c34)c2)cc1]

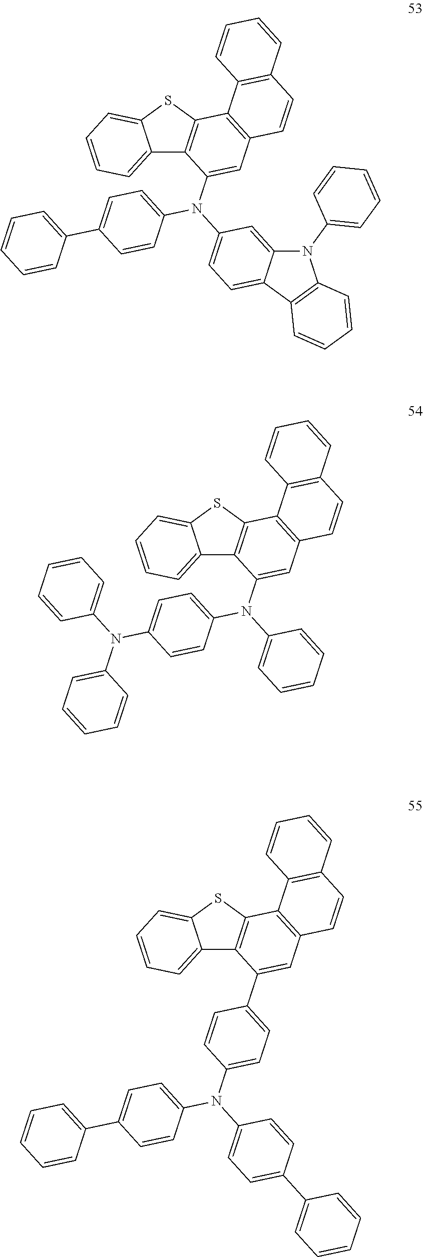 [SMILES: c1ccc(-c2ccc(N(c3ccc4c5ccccc5n(-c5ccccc5)c4c3)c3cc4ccc5ccccc5c4c4sc5ccccc5c34)cc2)cc1 ; c1ccc(N(c2ccccc2)c2ccc(N(c3ccccc3)c3cc4ccc5ccccc5c4c4sc5ccccc5c34)cc2)cc1 ; c1ccc(-c2ccc(N(c3ccc(-c4ccccc4)cc3)c3ccc(-c4cc5ccc6ccccc6c5c5sc6ccccc6c45)cc3)cc2)cc1]

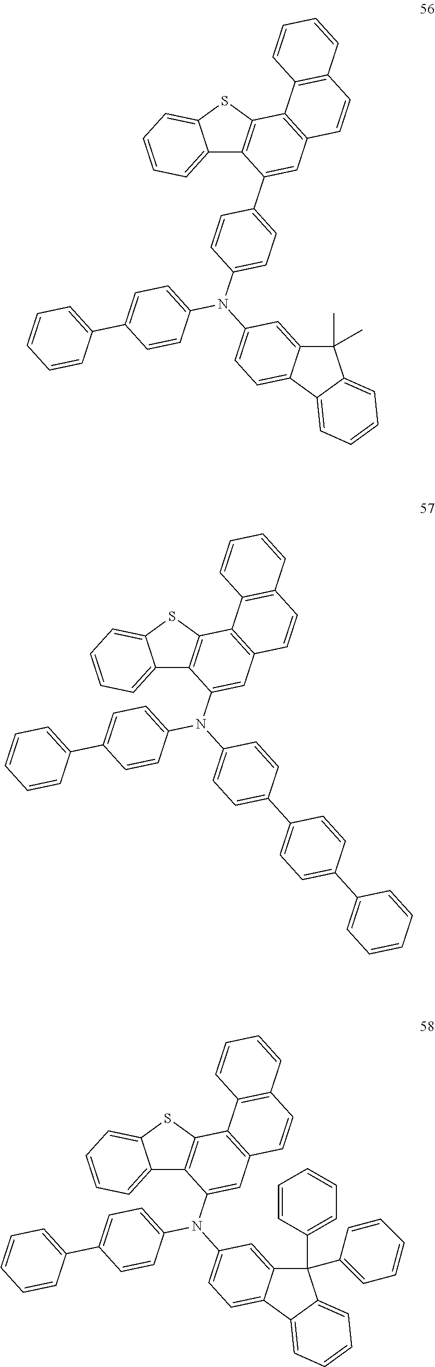 [SMILES: CC1(C)c2ccccc2-c2ccc(N(c3ccc(-c4ccccc4)cc3)c3ccc(-c4cc5ccc6ccccc6c5c5sc6ccccc6c45)cc3)cc21 ; c1ccc(-c2ccc(-c3ccc(N(c4ccc(-c5ccccc5)cc4)c4cc5ccc6ccccc6c5c5sc6ccccc6c45)cc3)cc2)cc1 ; c1ccc(-c2ccc(N(c3ccc4c(c3)C(c3ccccc3)(c3ccccc3)c3ccccc3-4)c3cc4ccc5ccccc5c4c4sc5ccccc5c34)cc2)cc1]

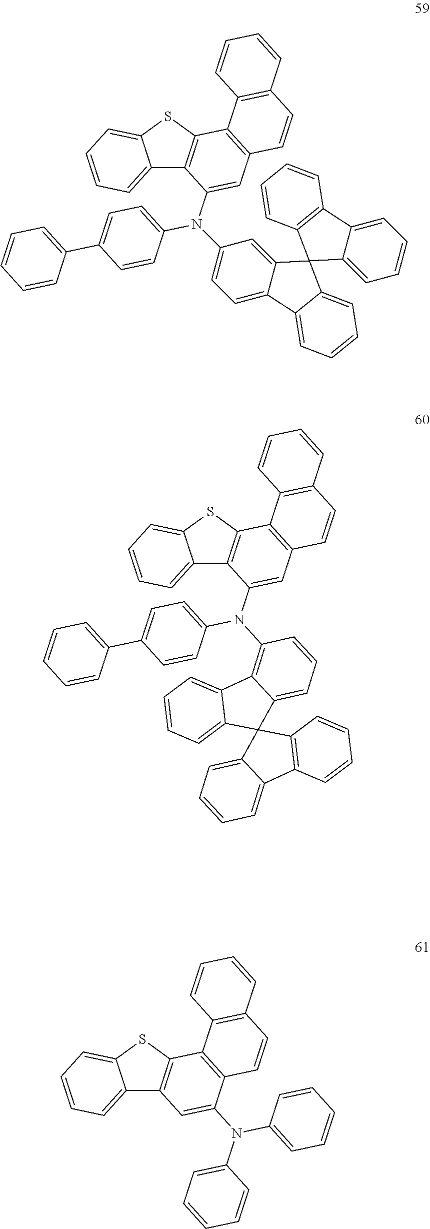 [SMILES: c1ccc(-c2ccc(N(c3ccc4c(c3)C3(c5ccccc5-c5ccccc53)c3ccccc3-4)c3cc4ccc5ccccc5c4c4sc5ccccc5c34)cc2)cc1 ; c1ccc(-c2ccc(N(c3cccc4c3-c3ccccc3C43c4ccccc4-c4ccccc43)c3cc4ccc5ccccc5c4c4sc5ccccc5c34)cc2)cc1 ; c1ccc(N(c2ccccc2)c2cc3c4ccccc4sc3c3c2ccc2ccccc23)cc1]

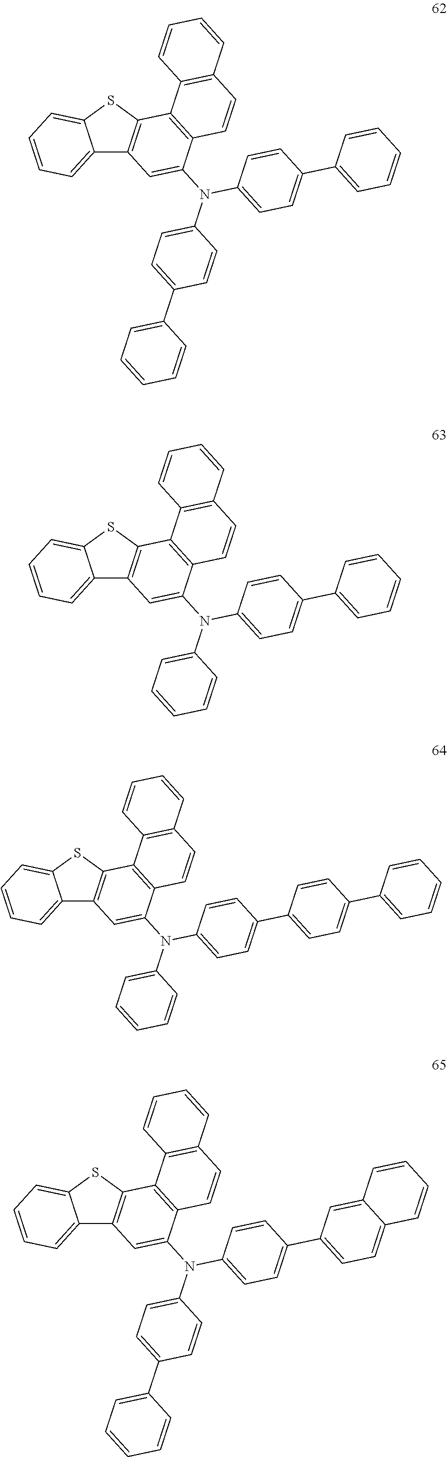 [SMILES: c1ccc(-c2ccc(N(c3ccc(-c4ccccc4)cc3)c3cc4c5ccccc5sc4c4c3ccc3ccccc34)cc2)cc1 ; c1ccc(-c2ccc(N(c3ccccc3)c3cc4c5ccccc5sc4c4c3ccc3ccccc34)cc2)cc1 ; c1ccc(-c2ccc(-c3ccc(N(c4ccccc4)c4cc5c6ccccc6sc5c5c4ccc4ccccc45)cc3)cc2)cc1 ; c1ccc(-c2ccc(N(c3ccc(-c4ccc5ccccc5c4)cc3)c3cc4c5ccccc5sc4c4c3ccc3ccccc34)cc2)cc1]

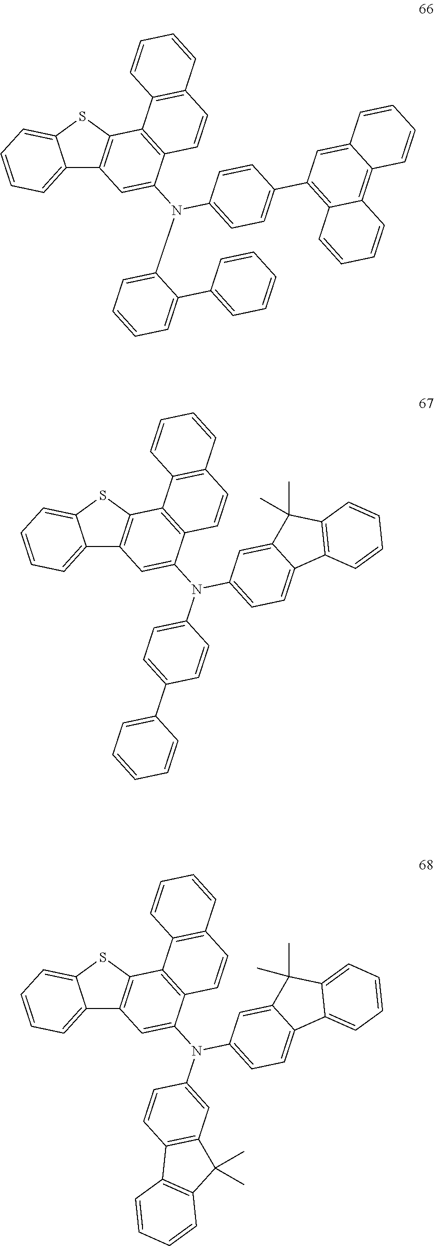 [SMILES: c1ccc(-c2ccccc2N(c2ccc(-c3cc4ccccc4c4ccccc34)cc2)c2cc3c4ccccc4sc3c3c2ccc2ccccc23)cc1 ; CC1(C)c2ccccc2-c2ccc(N(c3ccc(-c4ccccc4)cc3)c3cc4c5ccccc5sc4c4c3ccc3ccccc34)cc21 ; CC1(C)c2ccccc2-c2ccc(N(c3ccc4c(c3)C(C)(C)c3ccccc3-4)c3cc4c5ccccc5sc4c4c3ccc3ccccc34)cc21]

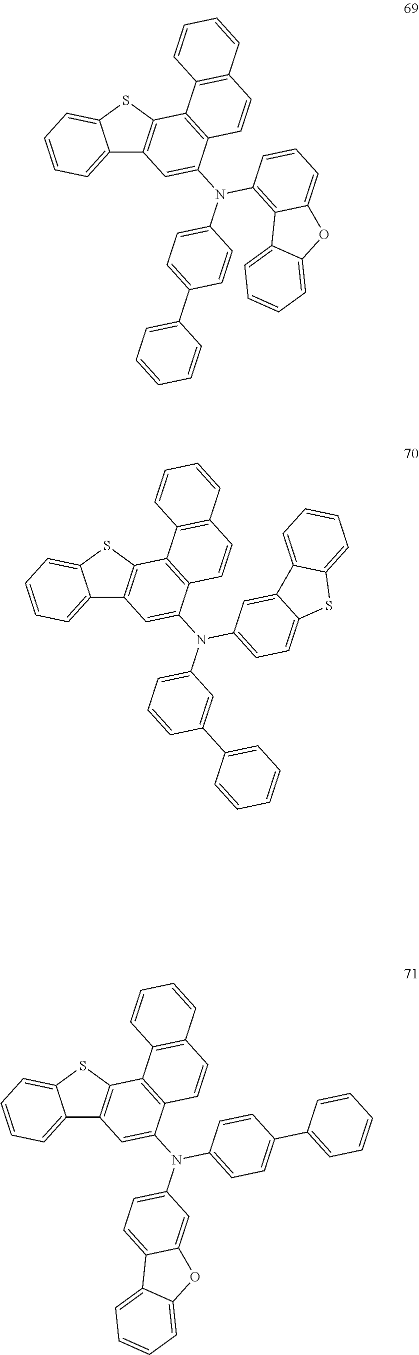 [SMILES: c1ccc(-c2ccc(N(c3cc4c5ccccc5sc4c4c3ccc3ccccc34)c3cccc4oc5ccccc5c34)cc2)cc1 ; c1ccc(-c2cccc(N(c3ccc4sc5ccccc5c4c3)c3cc4c5ccccc5sc4c4c3ccc3ccccc34)c2)cc1 ; c1ccc(-c2ccc(N(c3ccc4c(c3)oc3ccccc34)c3cc4c5ccccc5sc4c4c3ccc3ccccc34)cc2)cc1]

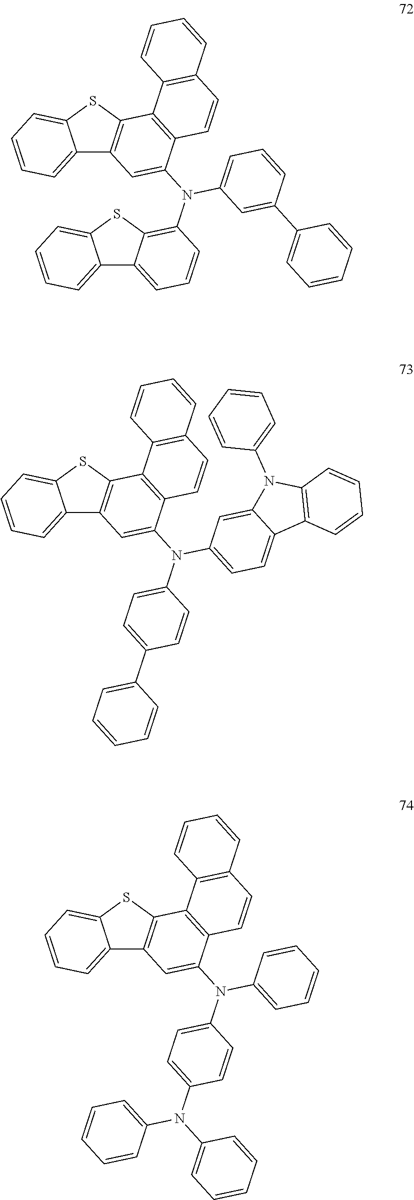 [SMILES: c1ccc(-c2cccc(N(c3cc4c5ccccc5sc4c4c3ccc3ccccc34)c3cccc4c3sc3ccccc34)c2)cc1 ; c1ccc(-c2ccc(N(c3ccc4c5ccccc5n(-c5ccccc5)c4c3)c3cc4c5ccccc5sc4c4c3ccc3ccccc34)cc2)cc1 ; c1ccc(N(c2ccccc2)c2ccc(N(c3ccccc3)c3cc4c5ccccc5sc4c4c3ccc3ccccc34)cc2)cc1]

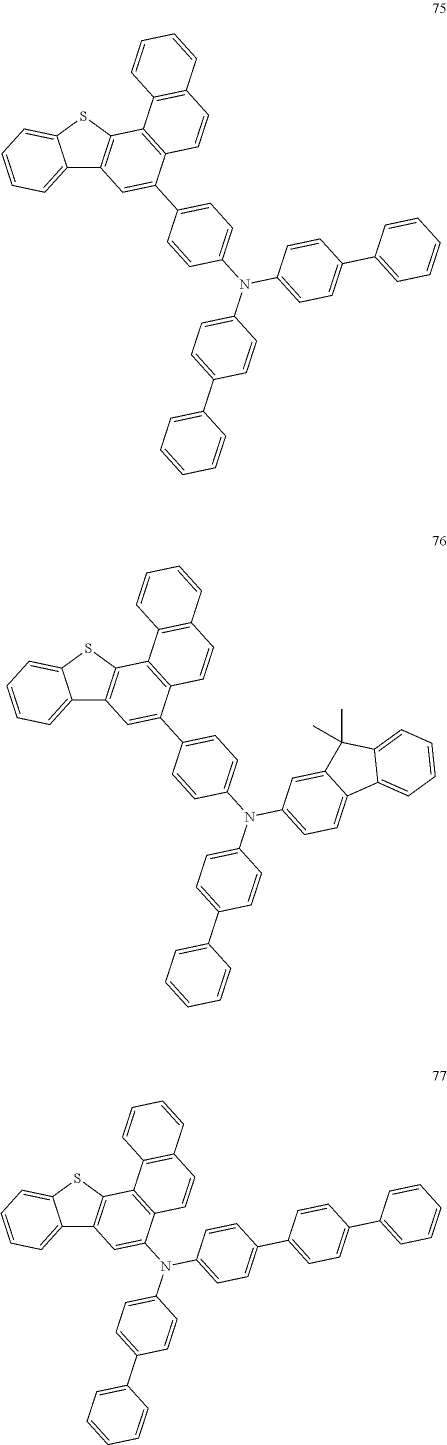 [SMILES: c1ccc(-c2ccc(N(c3ccc(-c4ccccc4)cc3)c3ccc(-c4cc5c6ccccc6sc5c5c4ccc4ccccc45)cc3)cc2)cc1 ; CC1(C)c2ccccc2-c2ccc(N(c3ccc(-c4ccccc4)cc3)c3ccc(-c4cc5c6ccccc6sc5c5c4ccc4ccccc45)cc3)cc21 ; c1ccc(-c2ccc(-c3ccc(N(c4ccc(-c5ccccc5)cc4)c4cc5c6ccccc6sc5c5c4ccc4ccccc45)cc3)cc2)cc1]

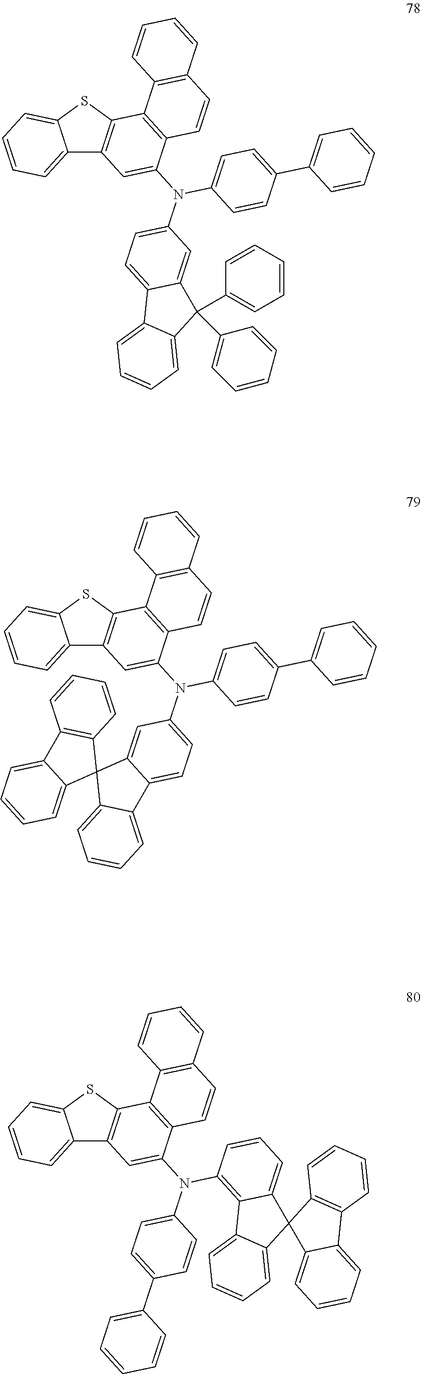 [SMILES: c1ccc(-c2ccc(N(c3ccc4c(c3)C(c3ccccc3)(c3ccccc3)c3ccccc3-4)c3cc4c5ccccc5sc4c4c3ccc3ccccc34)cc2)cc1 ; c1ccc(-c2ccc(N(c3ccc4c(c3)C3(c5ccccc5-c5ccccc53)c3ccccc3-4)c3cc4c5ccccc5sc4c4c3ccc3ccccc34)cc2)cc1 ; c1ccc(-c2ccc(N(c3cccc4c3-c3ccccc3C43c4ccccc4-c4ccccc43)c3cc4c5ccccc5sc4c4c3ccc3ccccc34)cc2)cc1]

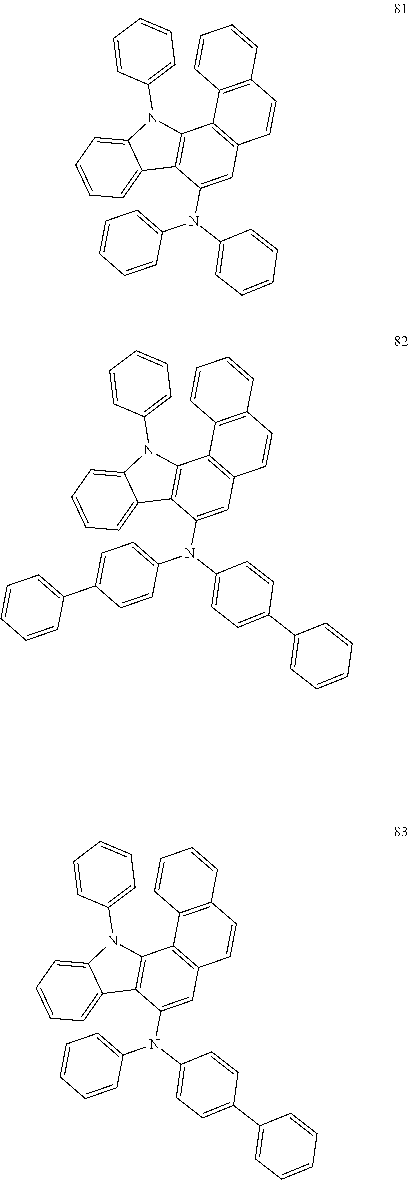 [SMILES: c1ccc(N(c2ccccc2)c2cc3ccc4ccccc4c3c3c2c2ccccc2n3-c2ccccc2)cc1 ; c1ccc(-c2ccc(N(c3ccc(-c4ccccc4)cc3)c3cc4ccc5ccccc5c4c4c3c3ccccc3n4-c3ccccc3)cc2)cc1 ; c1ccc(-c2ccc(N(c3ccccc3)c3cc4ccc5ccccc5c4c4c3c3ccccc3n4-c3ccccc3)cc2)cc1]

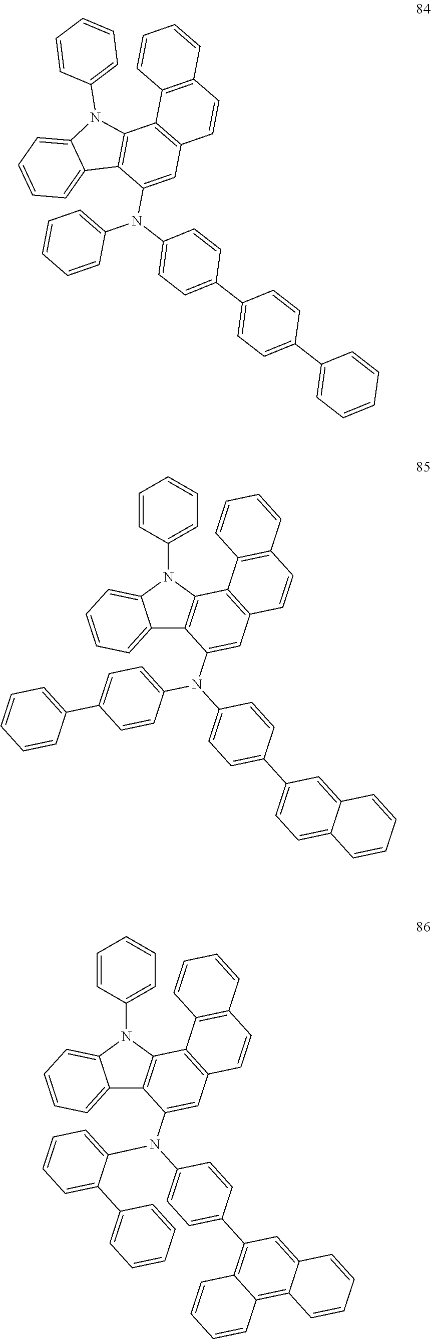 [SMILES: c1ccc(-c2ccc(-c3ccc(N(c4ccccc4)c4cc5ccc6ccccc6c5c5c4c4ccccc4n5-c4ccccc4)cc3)cc2)cc1 ; c1ccc(-c2ccc(N(c3ccc(-c4ccc5ccccc5c4)cc3)c3cc4ccc5ccccc5c4c4c3c3ccccc3n4-c3ccccc3)cc2)cc1 ; c1ccc(-c2ccccc2N(c2ccc(-c3cc4ccccc4c4ccccc34)cc2)c2cc3ccc4ccccc4c3c3c2c2ccccc2n3-c2ccccc2)cc1]

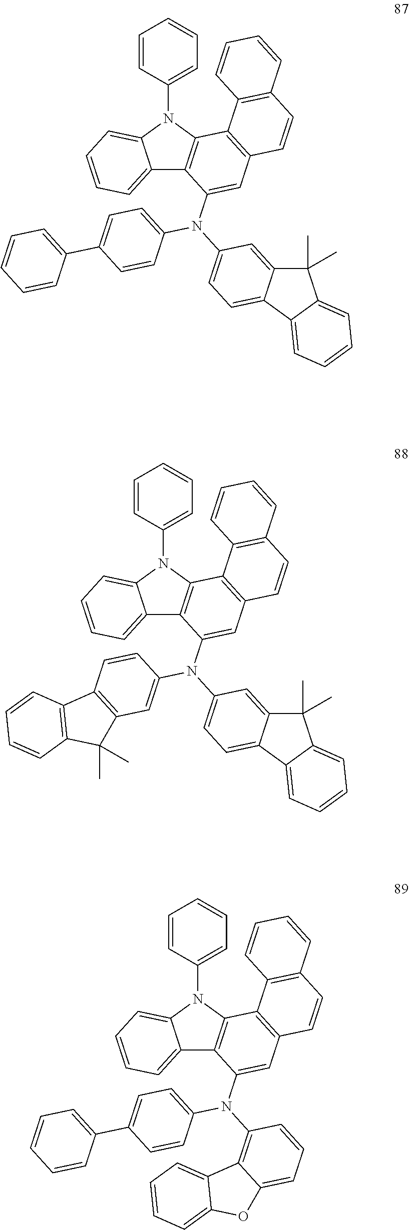 [SMILES: CC1(C)c2ccccc2-c2ccc(N(c3ccc(-c4ccccc4)cc3)c3cc4ccc5ccccc5c4c4c3c3ccccc3n4-c3ccccc3)cc21 ; CC1(C)c2ccccc2-c2ccc(N(c3ccc4c(c3)C(C)(C)c3ccccc3-4)c3cc4ccc5ccccc5c4c4c3c3ccccc3n4-c3ccccc3)cc21 ; c1ccc(-c2ccc(N(c3cccc4oc5ccccc5c34)c3cc4ccc5ccccc5c4c4c3c3ccccc3n4-c3ccccc3)cc2)cc1]

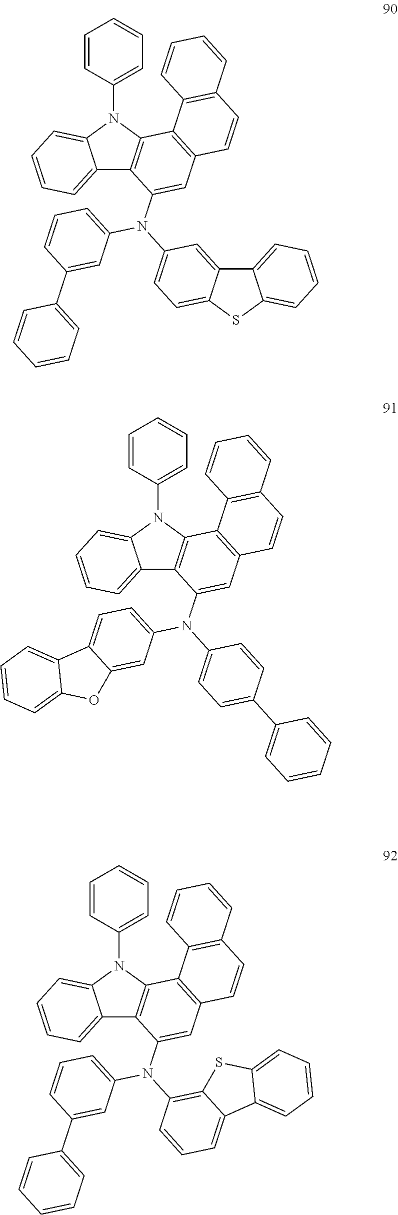 [SMILES: c1ccc(-c2cccc(N(c3ccc4sc5ccccc5c4c3)c3cc4ccc5ccccc5c4c4c3c3ccccc3n4-c3ccccc3)c2)cc1 ; c1ccc(-c2ccc(N(c3ccc4c(c3)oc3ccccc34)c3cc4ccc5ccccc5c4c4c3c3ccccc3n4-c3ccccc3)cc2)cc1 ; c1ccc(-c2cccc(N(c3cccc4c3sc3ccccc34)c3cc4ccc5ccccc5c4c4c3c3ccccc3n4-c3ccccc3)c2)cc1]

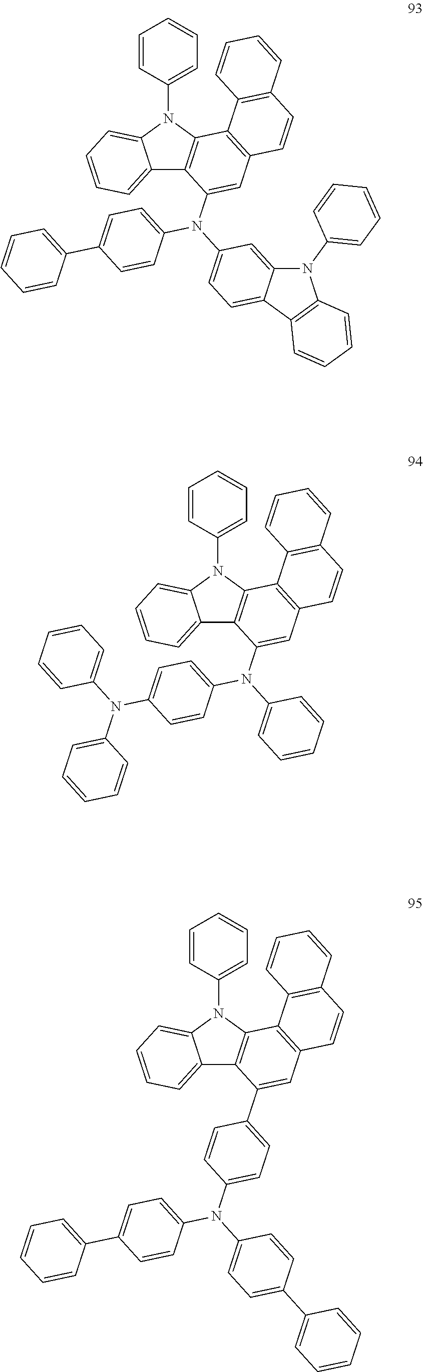 [SMILES: c1ccc(-c2ccc(N(c3ccc4c5ccccc5n(-c5ccccc5)c4c3)c3cc4ccc5ccccc5c4c4c3c3ccccc3n4-c3ccccc3)cc2)cc1 ; c1ccc(N(c2ccccc2)c2ccc(N(c3ccccc3)c3cc4ccc5ccccc5c4c4c3c3ccccc3n4-c3ccccc3)cc2)cc1 ; c1ccc(-c2ccc(N(c3ccc(-c4ccccc4)cc3)c3ccc(-c4cc5ccc6ccccc6c5c5c4c4ccccc4n5-c4ccccc4)cc3)cc2)cc1]

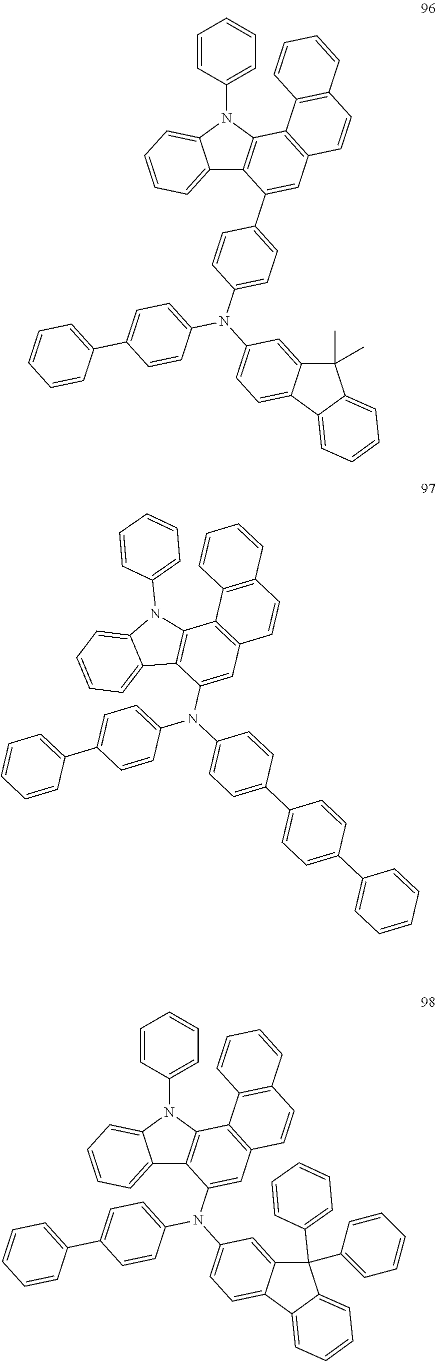 [SMILES: CC1(C)c2ccccc2-c2ccc(N(c3ccc(-c4ccccc4)cc3)c3ccc(-c4cc5ccc6ccccc6c5c5c4c4ccccc4n5-c4ccccc4)cc3)cc21 ; c1ccc(-c2ccc(-c3ccc(N(c4ccc(-c5ccccc5)cc4)c4cc5ccc6ccccc6c5c5c4c4ccccc4n5-c4ccccc4)cc3)cc2)cc1 ; c1ccc(-c2ccc(N(c3ccc4c(c3)C(c3ccccc3)(c3ccccc3)c3ccccc3-4)c3cc4ccc5ccccc5c4c4c3c3ccccc3n4-c3ccccc3)cc2)cc1]

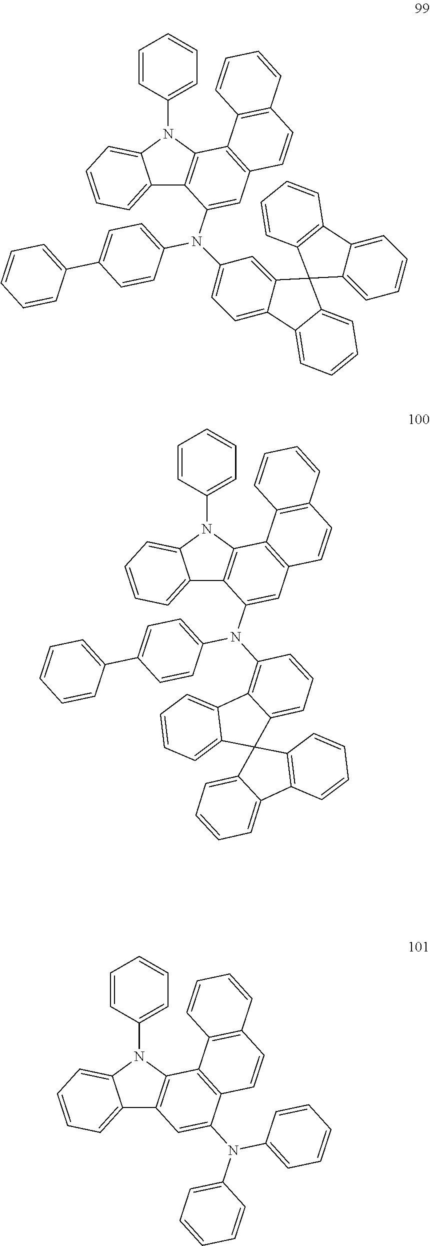 [SMILES: c1ccc(-c2ccc(N(c3ccc4c(c3)C3(c5ccccc5-c5ccccc53)c3ccccc3-4)c3cc4ccc5ccccc5c4c4c3c3ccccc3n4-c3ccccc3)cc2)cc1 ; c1ccc(-c2ccc(N(c3cccc4c3-c3ccccc3C43c4ccccc4-c4ccccc43)c3cc4ccc5ccccc5c4c4c3c3ccccc3n4-c3ccccc3)cc2)cc1 ; c1ccc(N(c2ccccc2)c2cc3c4ccccc4n(-c4ccccc4)c3c3c2ccc2ccccc23)cc1]

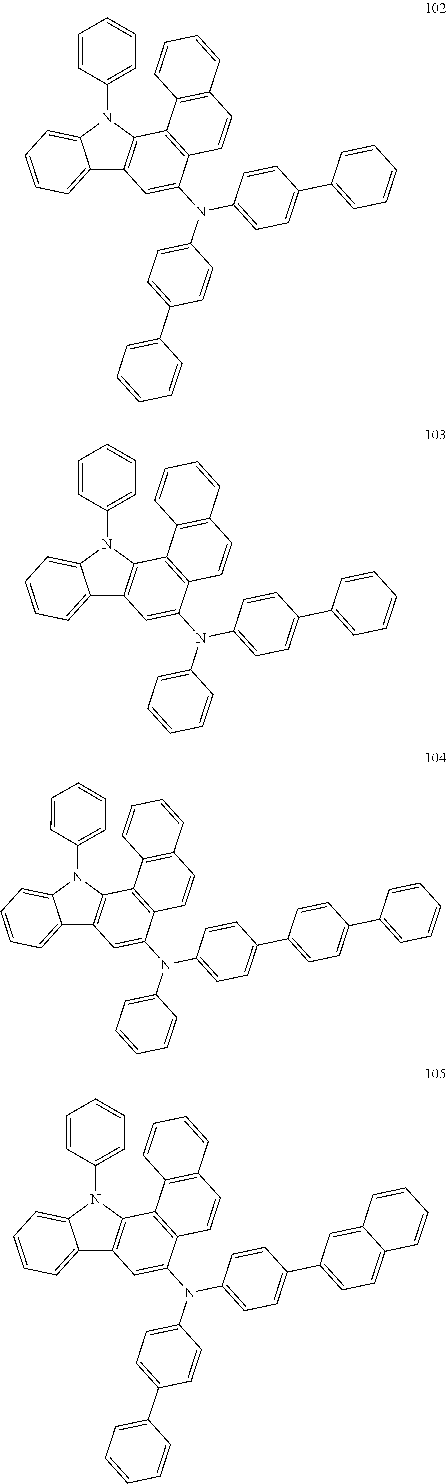 [SMILES: c1ccc(-c2ccc(N(c3ccc(-c4ccccc4)cc3)c3cc4c5ccccc5n(-c5ccccc5)c4c4c3ccc3ccccc34)cc2)cc1 ; c1ccc(-c2ccc(N(c3ccccc3)c3cc4c5ccccc5n(-c5ccccc5)c4c4c3ccc3ccccc34)cc2)cc1 ; c1ccc(-c2ccc(-c3ccc(N(c4ccccc4)c4cc5c6ccccc6n(-c6ccccc6)c5c5c4ccc4ccccc45)cc3)cc2)cc1 ; c1ccc(-c2ccc(N(c3ccc(-c4ccc5ccccc5c4)cc3)c3cc4c5ccccc5n(-c5ccccc5)c4c4c3ccc3ccccc34)cc2)cc1]

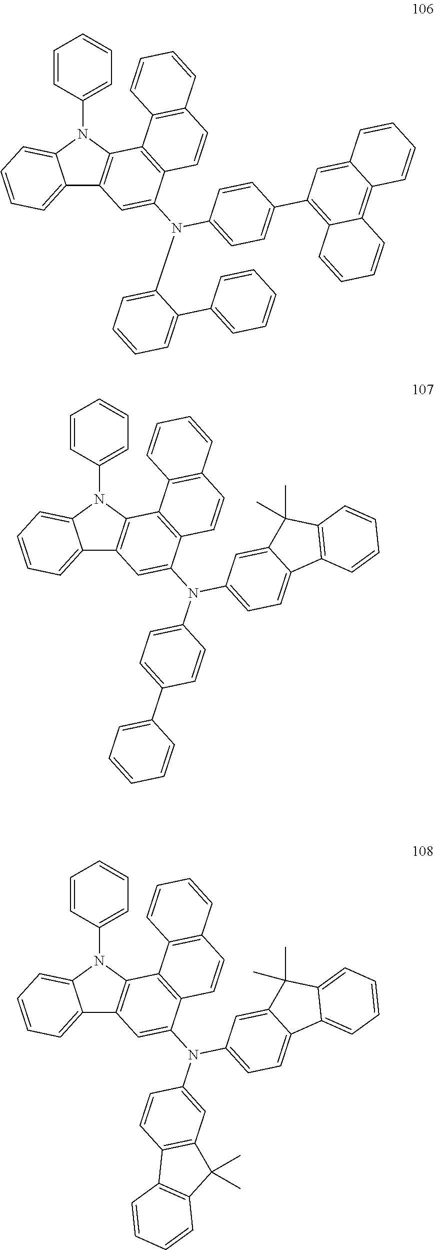 [SMILES: c1ccc(-c2ccccc2N(c2ccc(-c3cc4ccccc4c4ccccc34)cc2)c2cc3c4ccccc4n(-c4ccccc4)c3c3c2ccc2ccccc23)cc1 ; CC1(C)c2ccccc2-c2ccc(N(c3ccc(-c4ccccc4)cc3)c3cc4c5ccccc5n(-c5ccccc5)c4c4c3ccc3ccccc34)cc21 ; CC1(C)c2ccccc2-c2ccc(N(c3ccc4c(c3)C(C)(C)c3ccccc3-4)c3cc4c5ccccc5n(-c5ccccc5)c4c4c3ccc3ccccc34)cc21]

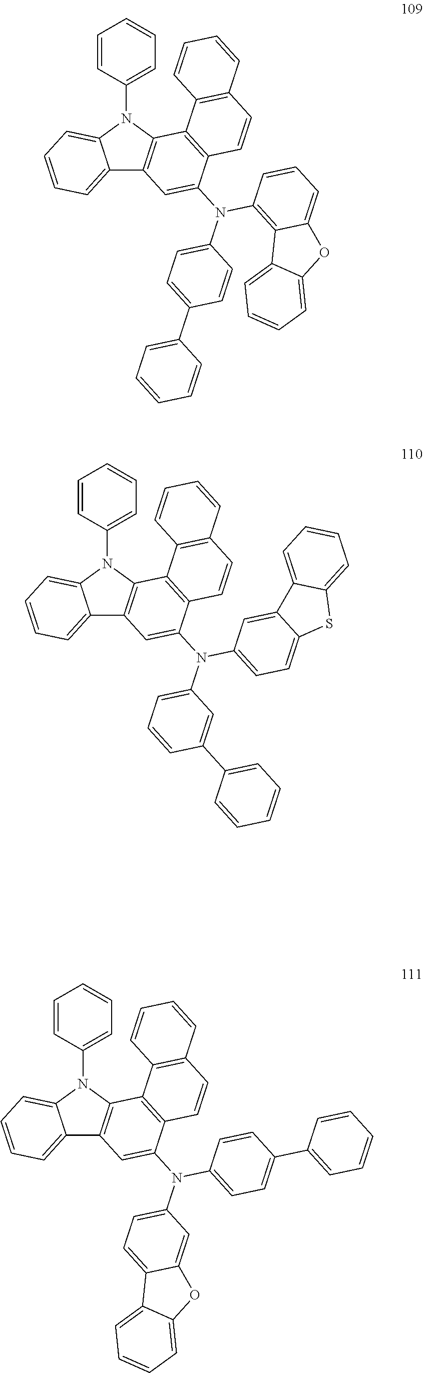 [SMILES: c1ccc(-c2ccc(N(c3cc4c5ccccc5n(-c5ccccc5)c4c4c3ccc3ccccc34)c3cccc4oc5ccccc5c34)cc2)cc1 ; c1ccc(-c2cccc(N(c3ccc4sc5ccccc5c4c3)c3cc4c5ccccc5n(-c5ccccc5)c4c4c3ccc3ccccc34)c2)cc1 ; c1ccc(-c2ccc(N(c3ccc4c(c3)oc3ccccc34)c3cc4c5ccccc5n(-c5ccccc5)c4c4c3ccc3ccccc34)cc2)cc1]

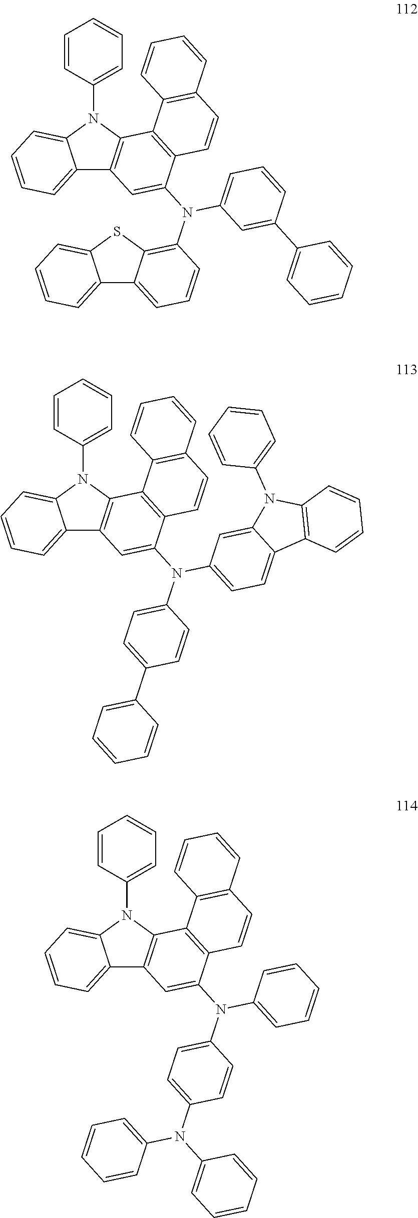 [SMILES: c1ccc(-c2cccc(N(c3cc4c5ccccc5n(-c5ccccc5)c4c4c3ccc3ccccc34)c3cccc4c3sc3ccccc34)c2)cc1 ; c1ccc(-c2ccc(N(c3ccc4c5ccccc5n(-c5ccccc5)c4c3)c3cc4c5ccccc5n(-c5ccccc5)c4c4c3ccc3ccccc34)cc2)cc1 ; c1ccc(N(c2ccccc2)c2ccc(N(c3ccccc3)c3cc4c5ccccc5n(-c5ccccc5)c4c4c3ccc3ccccc34)cc2)cc1]

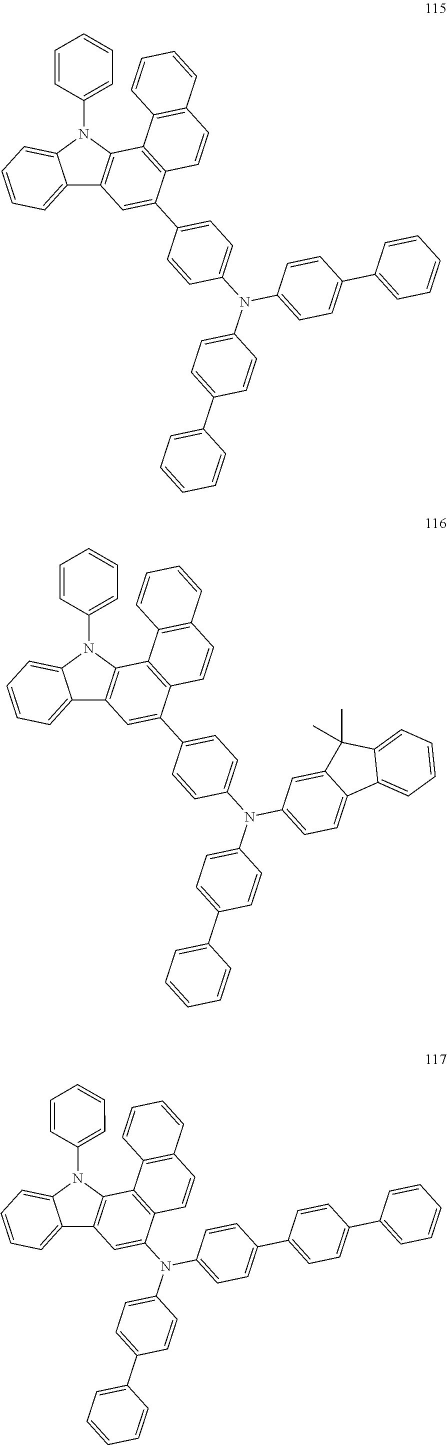 [SMILES: c1ccc(-c2ccc(N(c3ccc(-c4ccccc4)cc3)c3ccc(-c4cc5c6ccccc6n(-c6ccccc6)c5c5c4ccc4ccccc45)cc3)cc2)cc1 ; CC1(C)c2ccccc2-c2ccc(N(c3ccc(-c4ccccc4)cc3)c3ccc(-c4cc5c6ccccc6n(-c6ccccc6)c5c5c4ccc4ccccc45)cc3)cc21 ; c1ccc(-c2ccc(-c3ccc(N(c4ccc(-c5ccccc5)cc4)c4cc5c6ccccc6n(-c6ccccc6)c5c5c4ccc4ccccc45)cc3)cc2)cc1]

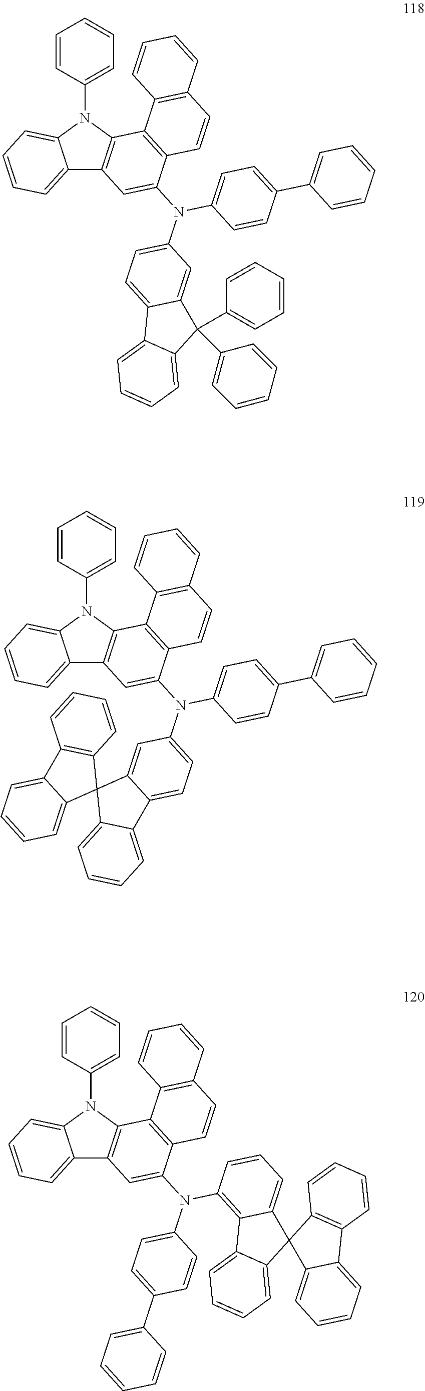 [SMILES: c1ccc(-c2ccc(N(c3ccc4c(c3)C(c3ccccc3)(c3ccccc3)c3ccccc3-4)c3cc4c5ccccc5n(-c5ccccc5)c4c4c3ccc3ccccc34)cc2)cc1 ; c1ccc(-c2ccc(N(c3ccc4c(c3)C3(c5ccccc5-c5ccccc53)c3ccccc3-4)c3cc4c5ccccc5n(-c5ccccc5)c4c4c3ccc3ccccc34)cc2)cc1 ; c1ccc(-c2ccc(N(c3cccc4c3-c3ccccc3C43c4ccccc4-c4ccccc43)c3cc4c5ccccc5n(-c5ccccc5)c4c4c3ccc3ccccc34)cc2)cc1]

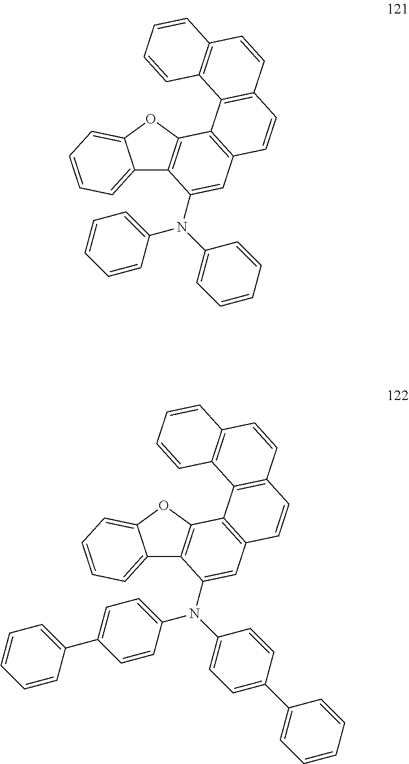 [SMILES: c1ccc(N(c2ccccc2)c2cc3ccc4ccc5ccccc5c4c3c3oc4ccccc4c23)cc1 ; c1ccc(-c2ccc(N(c3ccc(-c4ccccc4)cc3)c3cc4ccc5ccc6ccccc6c5c4c4oc5ccccc5c34)cc2)cc1]

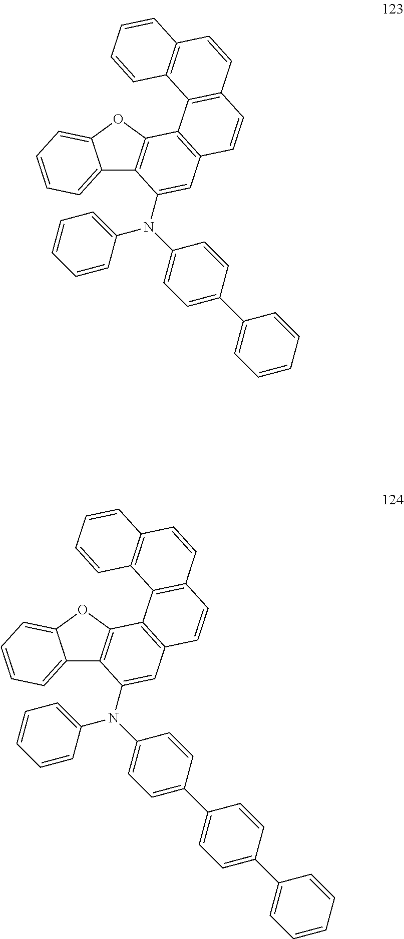 [SMILES: c1ccc(-c2ccc(N(c3ccccc3)c3cc4ccc5ccc6ccccc6c5c4c4oc5ccccc5c34)cc2)cc1 ; c1ccc(-c2ccc(-c3ccc(N(c4ccccc4)c4cc5ccc6ccc7ccccc7c6c5c5oc6ccccc6c45)cc3)cc2)cc1]

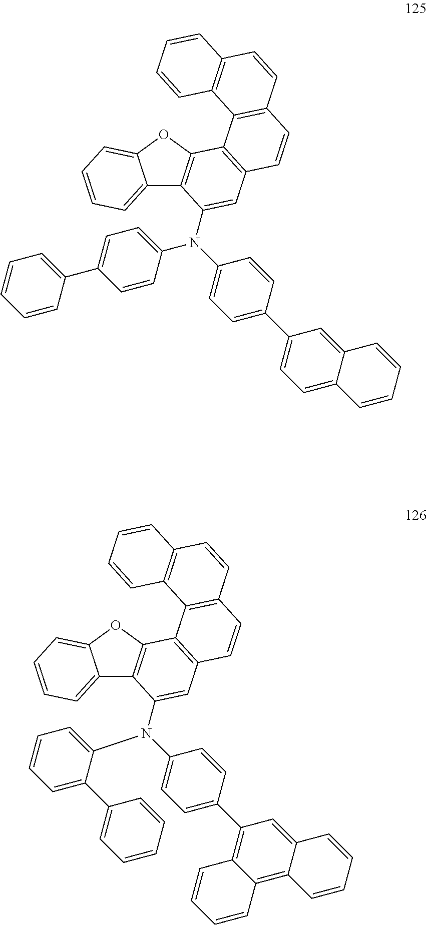 [SMILES: c1ccc(-c2ccc(N(c3ccc(-c4ccc5ccccc5c4)cc3)c3cc4ccc5ccc6ccccc6c5c4c4oc5ccccc5c34)cc2)cc1 ; c1ccc(-c2ccccc2N(c2ccc(-c3cc4ccccc4c4ccccc34)cc2)c2cc3ccc4ccc5ccccc5c4c3c3oc4ccccc4c23)cc1]

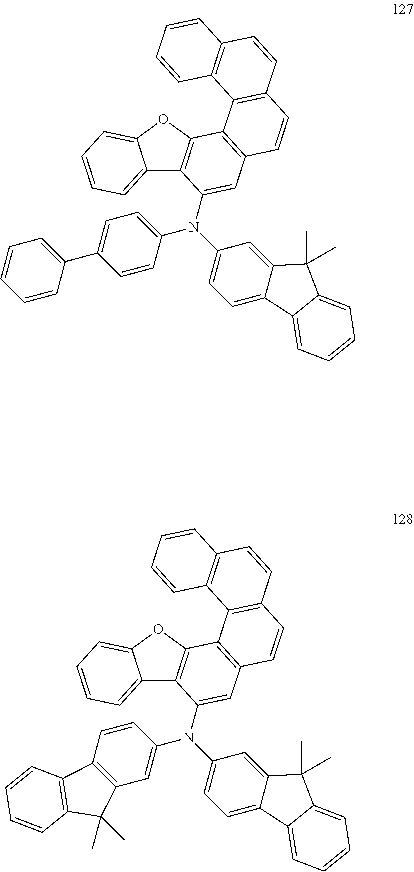 [SMILES: CC1(C)c2ccccc2-c2ccc(N(c3ccc(-c4ccccc4)cc3)c3cc4ccc5ccc6ccccc6c5c4c4oc5ccccc5c34)cc21 ; CC1(C)c2ccccc2-c2ccc(N(c3ccc4c(c3)C(C)(C)c3ccccc3-4)c3cc4ccc5ccc6ccccc6c5c4c4oc5ccccc5c34)cc21]

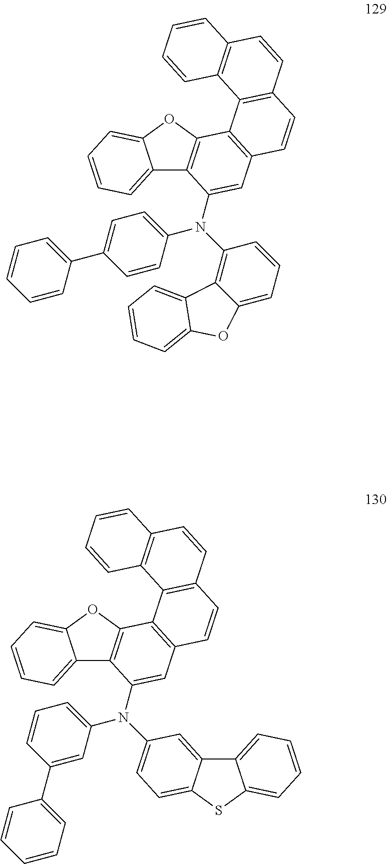 [SMILES: c1ccc(-c2ccc(N(c3cccc4oc5ccccc5c34)c3cc4ccc5ccc6ccccc6c5c4c4oc5ccccc5c34)cc2)cc1 ; c1ccc(-c2cccc(N(c3ccc4sc5ccccc5c4c3)c3cc4ccc5ccc6ccccc6c5c4c4oc5ccccc5c34)c2)cc1]

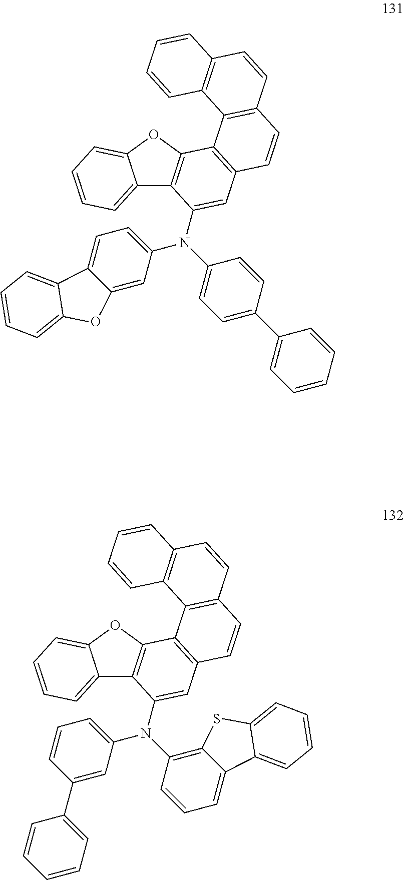 [SMILES: c1ccc(-c2ccc(N(c3ccc4c(c3)oc3ccccc34)c3cc4ccc5ccc6ccccc6c5c4c4oc5ccccc5c34)cc2)cc1 ; c1ccc(-c2cccc(N(c3cccc4c3sc3ccccc34)c3cc4ccc5ccc6ccccc6c5c4c4oc5ccccc5c34)c2)cc1]

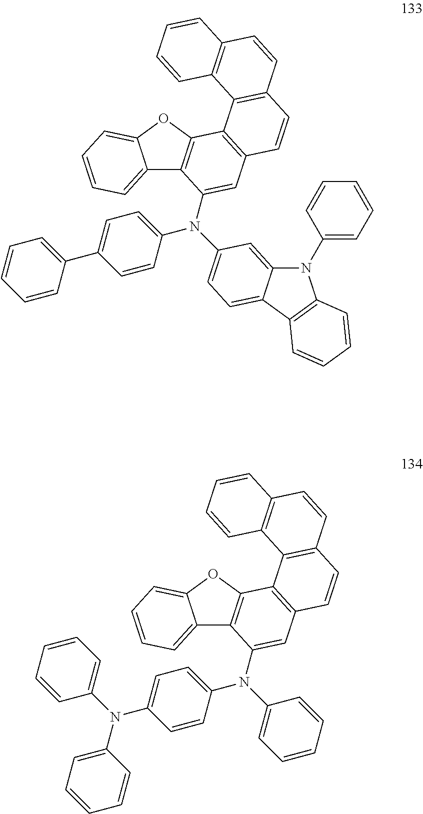 [SMILES: c1ccc(-c2ccc(N(c3ccc4c5ccccc5n(-c5ccccc5)c4c3)c3cc4ccc5ccc6ccccc6c5c4c4oc5ccccc5c34)cc2)cc1 ; c1ccc(N(c2ccccc2)c2ccc(N(c3ccccc3)c3cc4ccc5ccc6ccccc6c5c4c4oc5ccccc5c34)cc2)cc1]

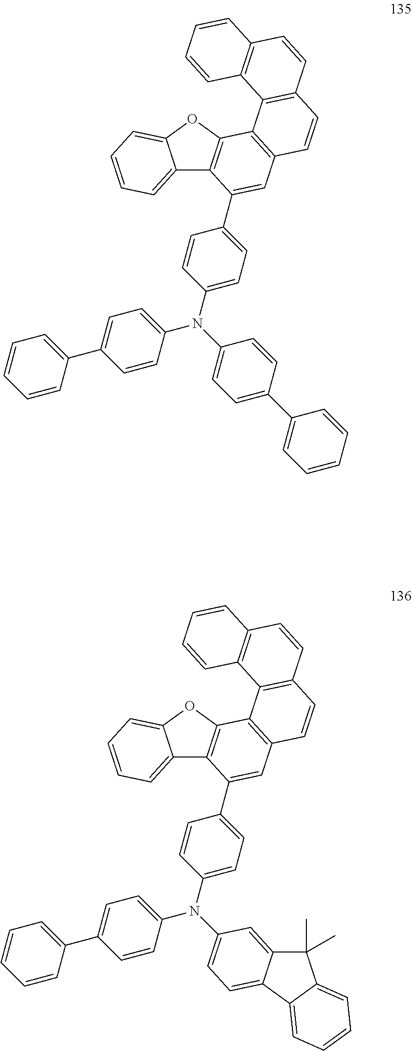 [SMILES: c1ccc(-c2ccc(N(c3ccc(-c4ccccc4)cc3)c3ccc(-c4cc5ccc6ccc7ccccc7c6c5c5oc6ccccc6c45)cc3)cc2)cc1 ; CC1(C)c2ccccc2-c2ccc(N(c3ccc(-c4ccccc4)cc3)c3ccc(-c4cc5ccc6ccc7ccccc7c6c5c5oc6ccccc6c45)cc3)cc21]

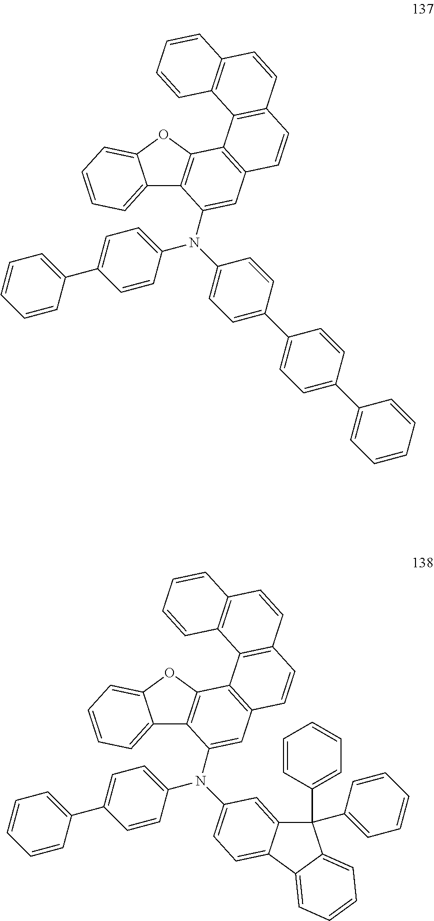 [SMILES: c1ccc(-c2ccc(-c3ccc(N(c4ccc(-c5ccccc5)cc4)c4cc5ccc6ccc7ccccc7c6c5c5oc6ccccc6c45)cc3)cc2)cc1 ; c1ccc(-c2ccc(N(c3ccc4c(c3)C(c3ccccc3)(c3ccccc3)c3ccccc3-4)c3cc4ccc5ccc6ccccc6c5c4c4oc5ccccc5c34)cc2)cc1]

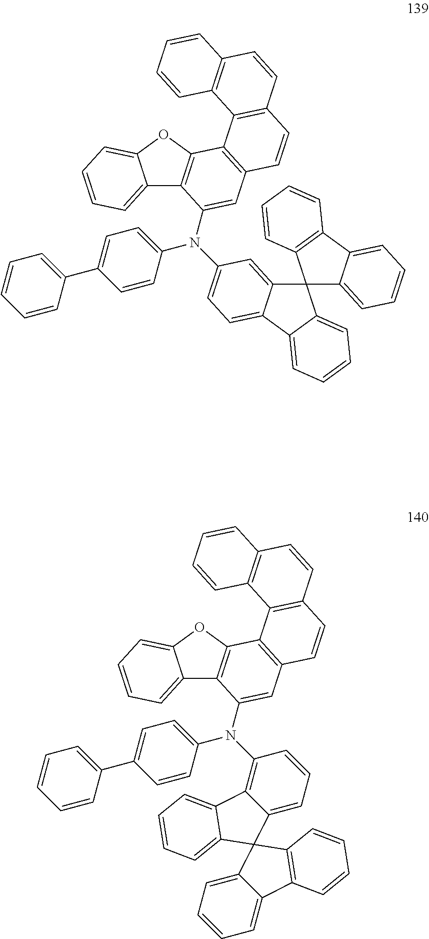 [SMILES: c1ccc(-c2ccc(N(c3ccc4c(c3)C3(c5ccccc5-c5ccccc53)c3ccccc3-4)c3cc4ccc5ccc6ccccc6c5c4c4oc5ccccc5c34)cc2)cc1 ; c1ccc(-c2ccc(N(c3cccc4c3-c3ccccc3C43c4ccccc4-c4ccccc43)c3cc4ccc5ccc6ccccc6c5c4c4oc5ccccc5c34)cc2)cc1]

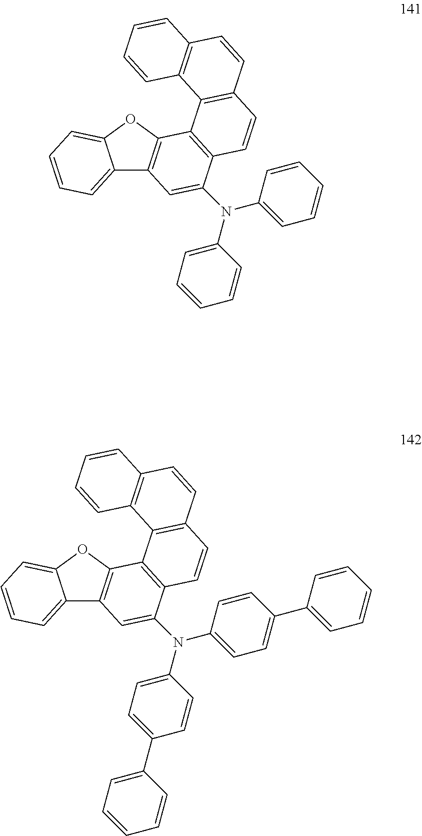 [SMILES: c1ccc(N(c2ccccc2)c2cc3c4ccccc4oc3c3c2ccc2ccc4ccccc4c23)cc1 ; c1ccc(-c2ccc(N(c3ccc(-c4ccccc4)cc3)c3cc4c5ccccc5oc4c4c3ccc3ccc5ccccc5c34)cc2)cc1]

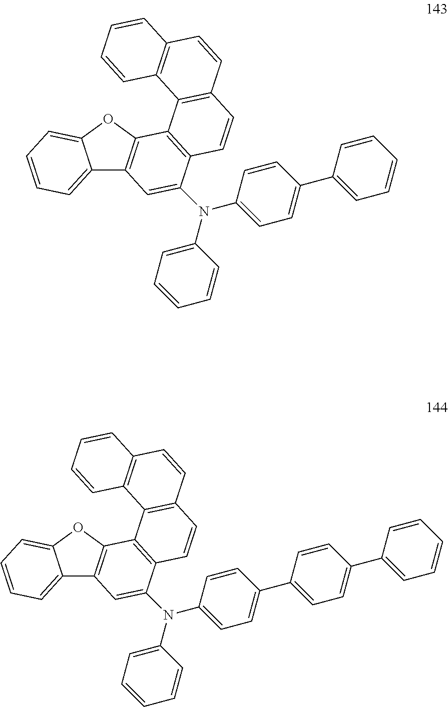 [SMILES: c1ccc(-c2ccc(N(c3ccccc3)c3cc4c5ccccc5oc4c4c3ccc3ccc5ccccc5c34)cc2)cc1 ; c1ccc(-c2ccc(-c3ccc(N(c4ccccc4)c4cc5c6ccccc6oc5c5c4ccc4ccc6ccccc6c45)cc3)cc2)cc1]

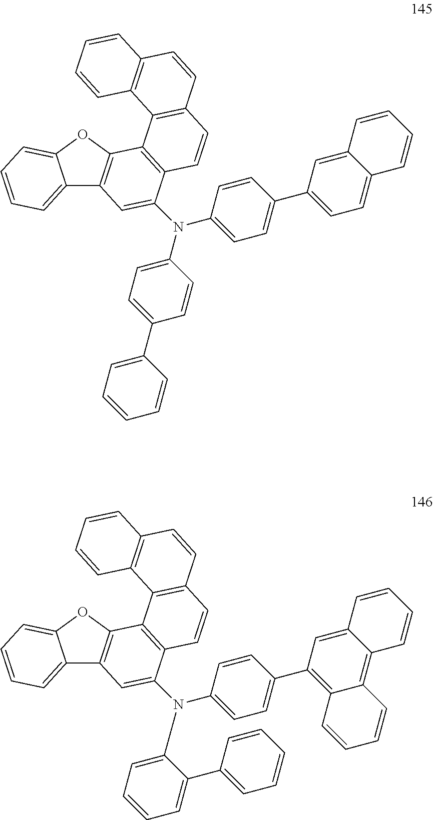 [SMILES: c1ccc(-c2ccc(N(c3ccc(-c4ccc5ccccc5c4)cc3)c3cc4c5ccccc5oc4c4c3ccc3ccc5ccccc5c34)cc2)cc1 ; c1ccc(-c2ccccc2N(c2ccc(-c3cc4ccccc4c4ccccc34)cc2)c2cc3c4ccccc4oc3c3c2ccc2ccc4ccccc4c23)cc1]

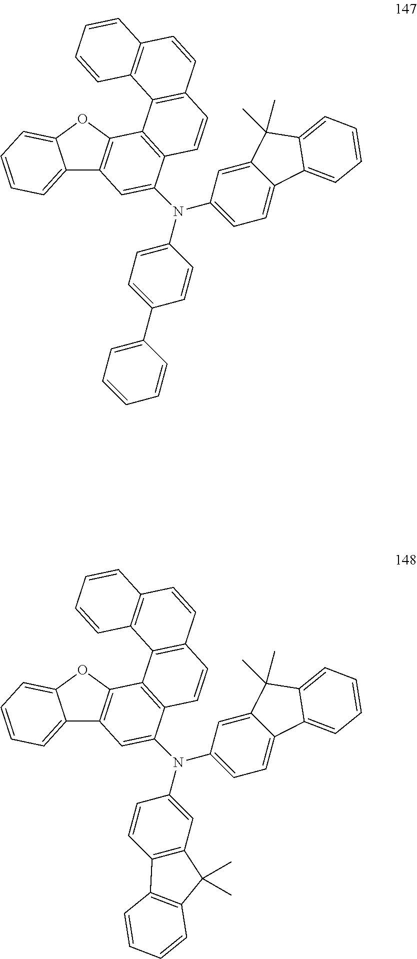 [SMILES: CC1(C)c2ccccc2-c2ccc(N(c3ccc(-c4ccccc4)cc3)c3cc4c5ccccc5oc4c4c3ccc3ccc5ccccc5c34)cc21 ; CC1(C)c2ccccc2-c2ccc(N(c3ccc4c(c3)C(C)(C)c3ccccc3-4)c3cc4c5ccccc5oc4c4c3ccc3ccc5ccccc5c34)cc21]

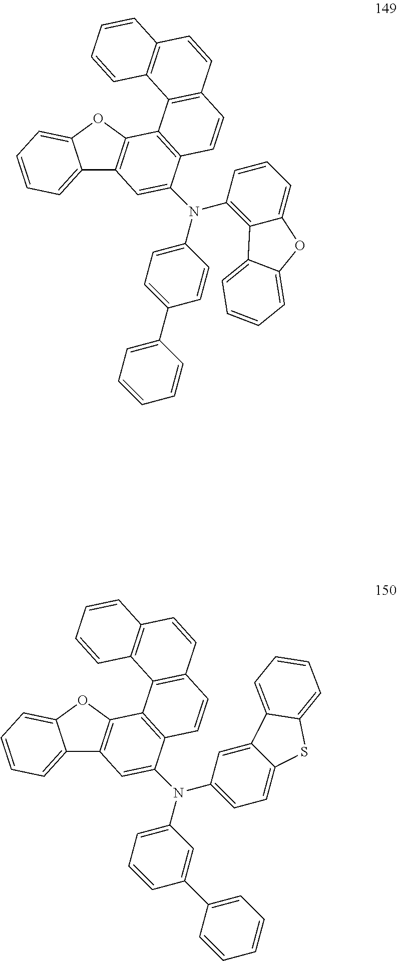 [SMILES: c1ccc(-c2ccc(N(c3cc4c5ccccc5oc4c4c3ccc3ccc5ccccc5c34)c3cccc4oc5ccccc5c34)cc2)cc1 ; c1ccc(-c2cccc(N(c3ccc4sc5ccccc5c4c3)c3cc4c5ccccc5oc4c4c3ccc3ccc5ccccc5c34)c2)cc1]

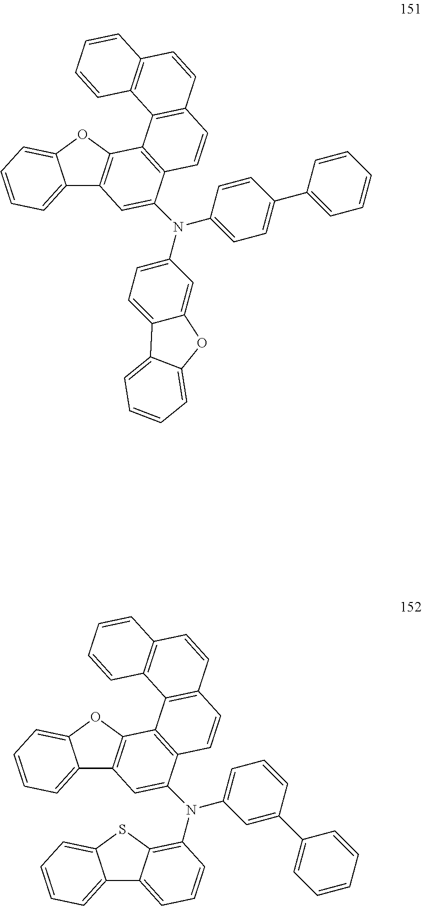 [SMILES: c1ccc(-c2ccc(N(c3ccc4c(c3)oc3ccccc34)c3cc4c5ccccc5oc4c4c3ccc3ccc5ccccc5c34)cc2)cc1 ; c1ccc(-c2cccc(N(c3cc4c5ccccc5oc4c4c3ccc3ccc5ccccc5c34)c3cccc4c3sc3ccccc34)c2)cc1]

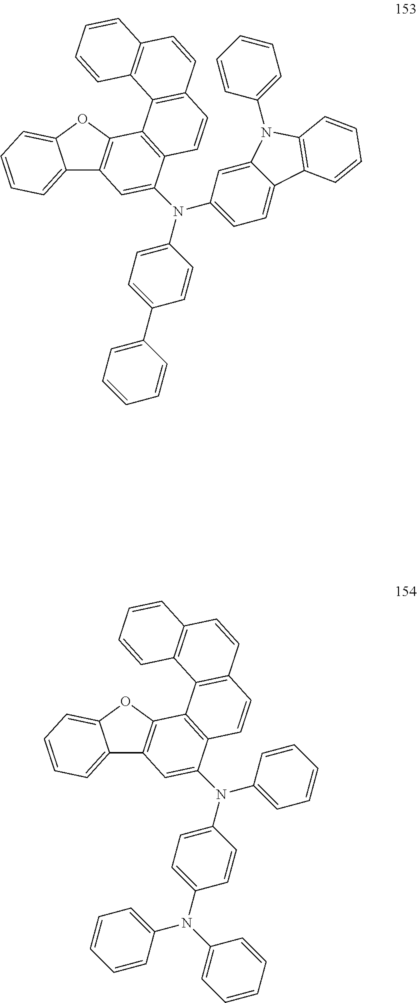 [SMILES: c1ccc(-c2ccc(N(c3ccc4c5ccccc5n(-c5ccccc5)c4c3)c3cc4c5ccccc5oc4c4c3ccc3ccc5ccccc5c34)cc2)cc1 ; c1ccc(N(c2ccccc2)c2ccc(N(c3ccccc3)c3cc4c5ccccc5oc4c4c3ccc3ccc5ccccc5c34)cc2)cc1]

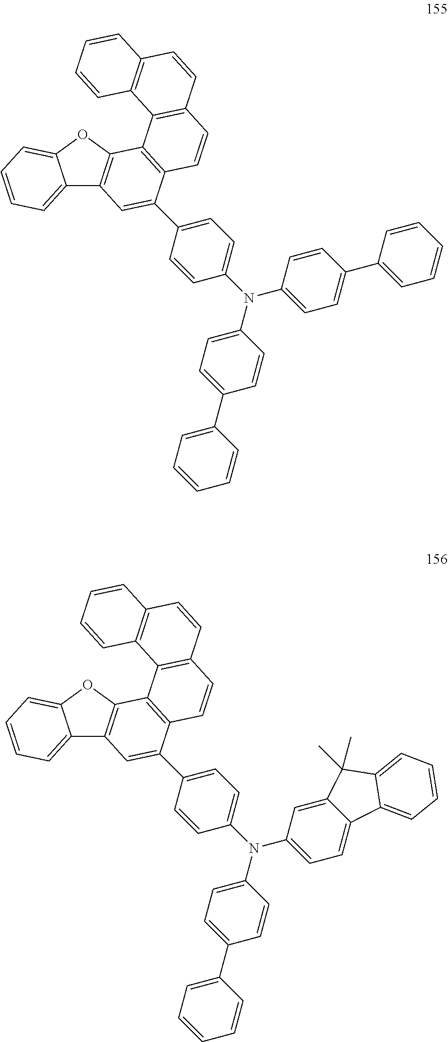 [SMILES: c1ccc(-c2ccc(N(c3ccc(-c4ccccc4)cc3)c3ccc(-c4cc5c6ccccc6oc5c5c4ccc4ccc6ccccc6c45)cc3)cc2)cc1 ; CC1(C)c2ccccc2-c2ccc(N(c3ccc(-c4ccccc4)cc3)c3ccc(-c4cc5c6ccccc6oc5c5c4ccc4ccc6ccccc6c45)cc3)cc21]

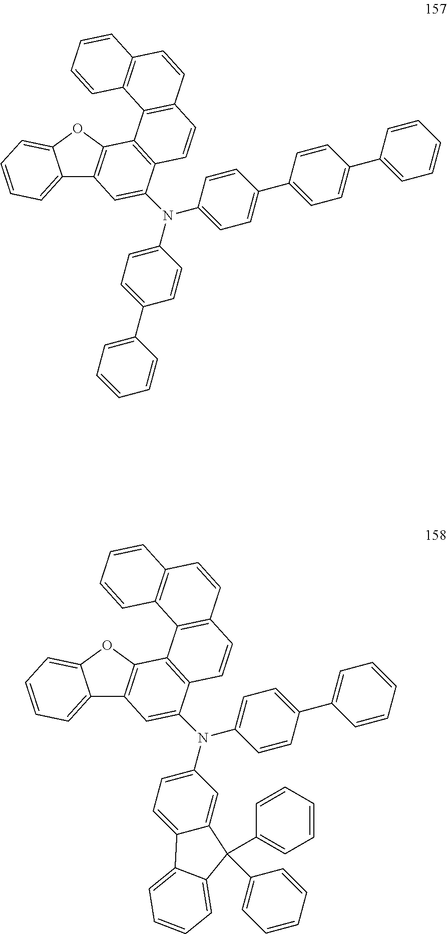 [SMILES: c1ccc(-c2ccc(-c3ccc(N(c4ccc(-c5ccccc5)cc4)c4cc5c6ccccc6oc5c5c4ccc4ccc6ccccc6c45)cc3)cc2)cc1 ; c1ccc(-c2ccc(N(c3ccc4c(c3)C(c3ccccc3)(c3ccccc3)c3ccccc3-4)c3cc4c5ccccc5oc4c4c3ccc3ccc5ccccc5c34)cc2)cc1]

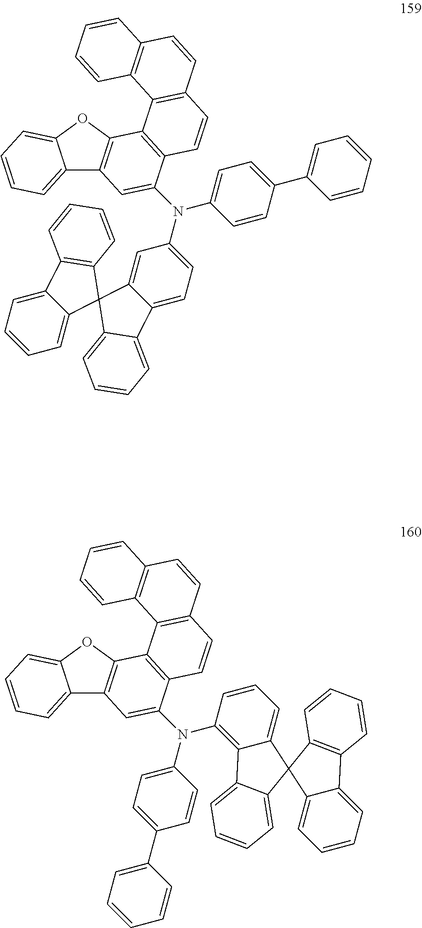 [SMILES: c1ccc(-c2ccc(N(c3ccc4c(c3)C3(c5ccccc5-c5ccccc53)c3ccccc3-4)c3cc4c5ccccc5oc4c4c3ccc3ccc5ccccc5c34)cc2)cc1 ; c1ccc(-c2ccc(N(c3cccc4c3-c3ccccc3C43c4ccccc4-c4ccccc43)c3cc4c5ccccc5oc4c4c3ccc3ccc5ccccc5c34)cc2)cc1]

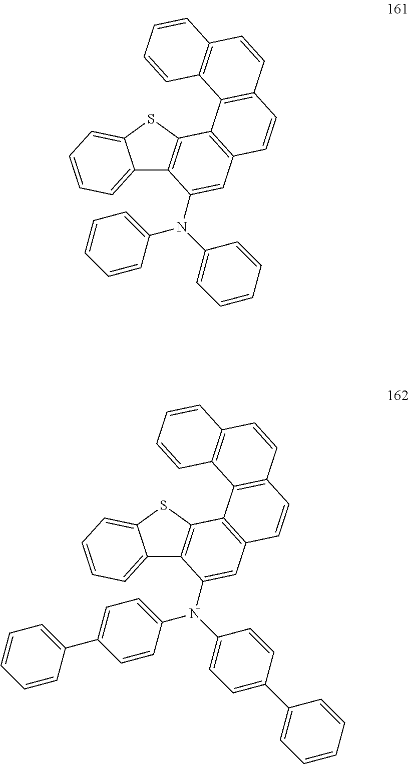 [SMILES: c1ccc(N(c2ccccc2)c2cc3ccc4ccc5ccccc5c4c3c3sc4ccccc4c23)cc1 ; c1ccc(-c2ccc(N(c3ccc(-c4ccccc4)cc3)c3cc4ccc5ccc6ccccc6c5c4c4sc5ccccc5c34)cc2)cc1]

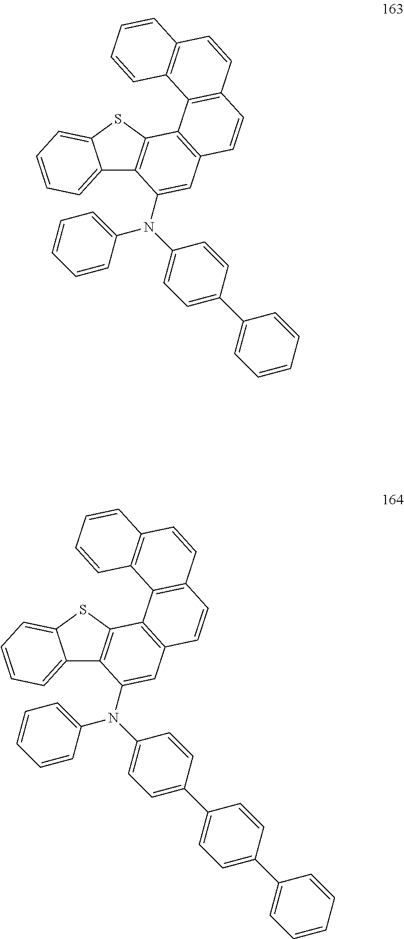 [SMILES: c1ccc(-c2ccc(N(c3ccccc3)c3cc4ccc5ccc6ccccc6c5c4c4sc5ccccc5c34)cc2)cc1 ; c1ccc(-c2ccc(-c3ccc(N(c4ccccc4)c4cc5ccc6ccc7ccccc7c6c5c5sc6ccccc6c45)cc3)cc2)cc1]

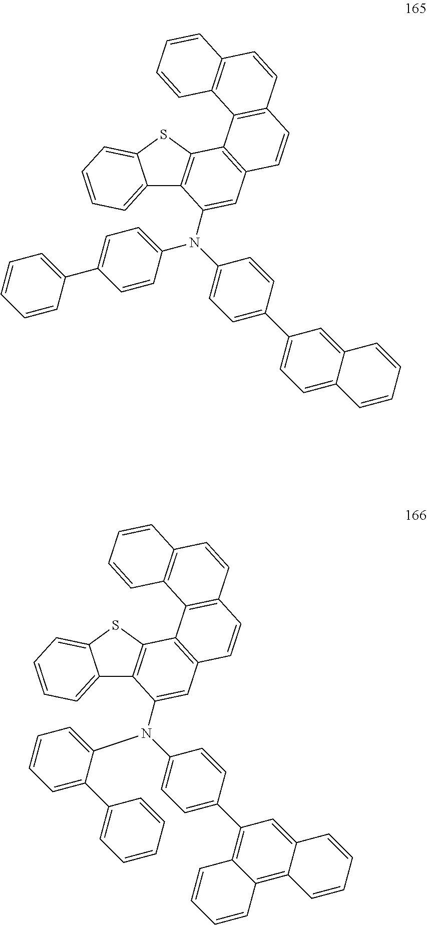 [SMILES: c1ccc(-c2ccc(N(c3ccc(-c4ccc5ccccc5c4)cc3)c3cc4ccc5ccc6ccccc6c5c4c4sc5ccccc5c34)cc2)cc1 ; c1ccc(-c2ccccc2N(c2ccc(-c3cc4ccccc4c4ccccc34)cc2)c2cc3ccc4ccc5ccccc5c4c3c3sc4ccccc4c23)cc1]

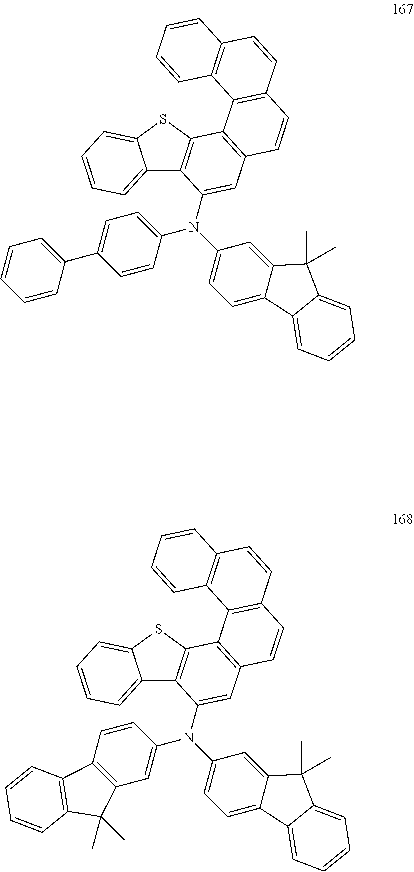 [SMILES: CC1(C)c2ccccc2-c2ccc(N(c3ccc(-c4ccccc4)cc3)c3cc4ccc5ccc6ccccc6c5c4c4sc5ccccc5c34)cc21 ; CC1(C)c2ccccc2-c2ccc(N(c3ccc4c(c3)C(C)(C)c3ccccc3-4)c3cc4ccc5ccc6ccccc6c5c4c4sc5ccccc5c34)cc21]

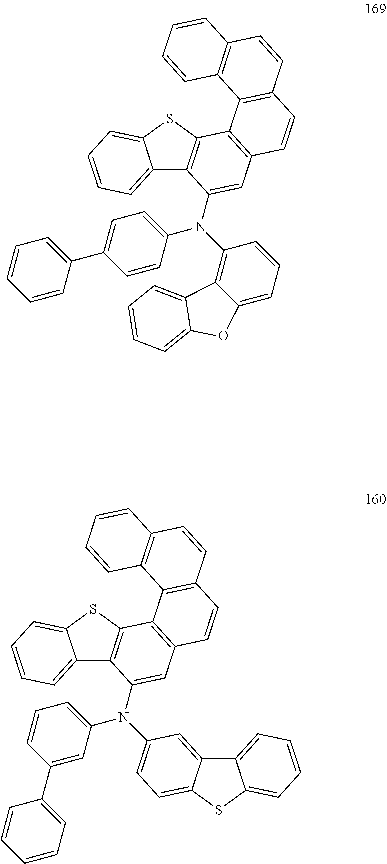 [SMILES: c1ccc(-c2ccc(N(c3cccc4oc5ccccc5c34)c3cc4ccc5ccc6ccccc6c5c4c4sc5ccccc5c34)cc2)cc1 ; c1ccc(-c2cccc(N(c3ccc4sc5ccccc5c4c3)c3cc4ccc5ccc6ccccc6c5c4c4sc5ccccc5c34)c2)cc1]

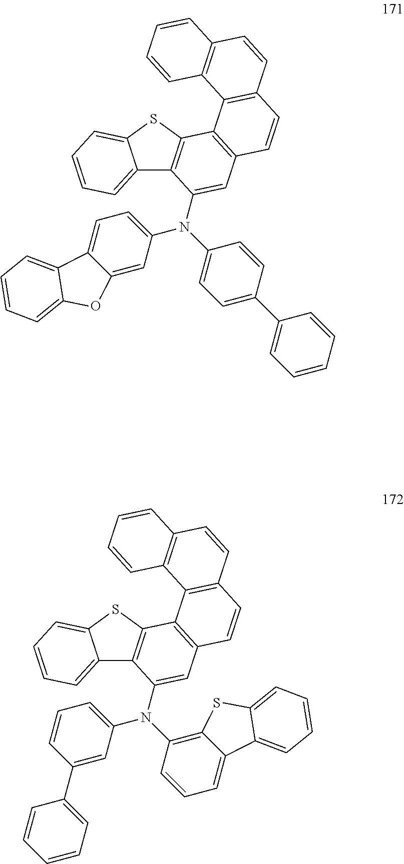 [SMILES: c1ccc(-c2ccc(N(c3ccc4c(c3)oc3ccccc34)c3cc4ccc5ccc6ccccc6c5c4c4sc5ccccc5c34)cc2)cc1 ; c1ccc(-c2cccc(N(c3cccc4c3sc3ccccc34)c3cc4ccc5ccc6ccccc6c5c4c4sc5ccccc5c34)c2)cc1]

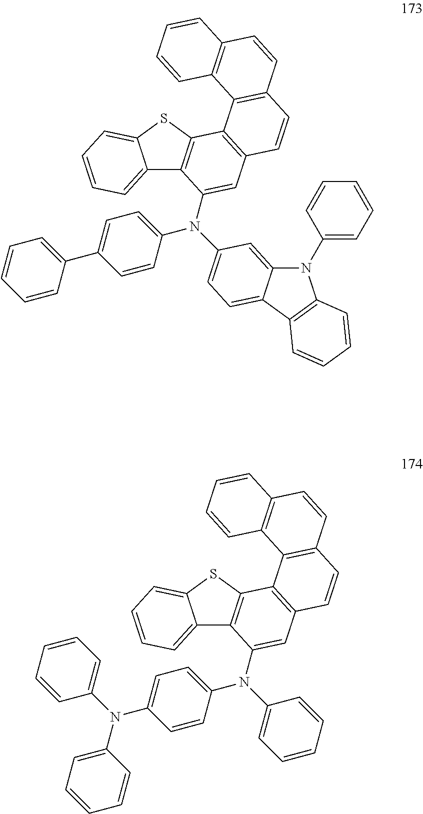 [SMILES: c1ccc(-c2ccc(N(c3ccc4c5ccccc5n(-c5ccccc5)c4c3)c3cc4ccc5ccc6ccccc6c5c4c4sc5ccccc5c34)cc2)cc1 ; c1ccc(N(c2ccccc2)c2ccc(N(c3ccccc3)c3cc4ccc5ccc6ccccc6c5c4c4sc5ccccc5c34)cc2)cc1]

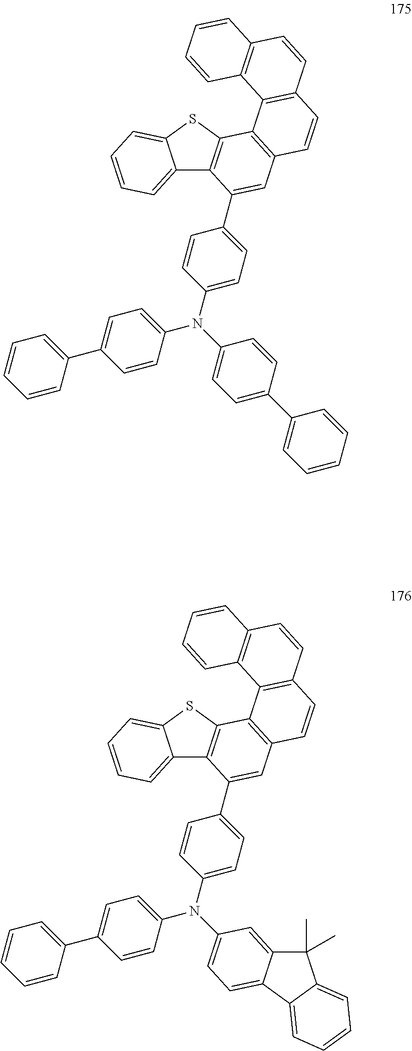 [SMILES: c1ccc(-c2ccc(N(c3ccc(-c4ccccc4)cc3)c3ccc(-c4cc5ccc6ccc7ccccc7c6c5c5sc6ccccc6c45)cc3)cc2)cc1 ; CC1(C)c2ccccc2-c2ccc(N(c3ccc(-c4ccccc4)cc3)c3ccc(-c4cc5ccc6ccc7ccccc7c6c5c5sc6ccccc6c45)cc3)cc21]

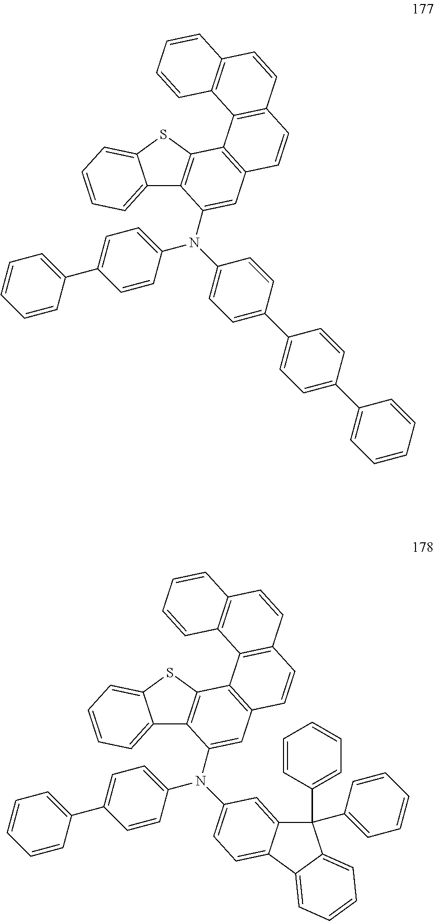 [SMILES: c1ccc(-c2ccc(-c3ccc(N(c4ccc(-c5ccccc5)cc4)c4cc5ccc6ccc7ccccc7c6c5c5sc6ccccc6c45)cc3)cc2)cc1 ; c1ccc(-c2ccc(N(c3ccc4c(c3)C(c3ccccc3)(c3ccccc3)c3ccccc3-4)c3cc4ccc5ccc6ccccc6c5c4c4sc5ccccc5c34)cc2)cc1]

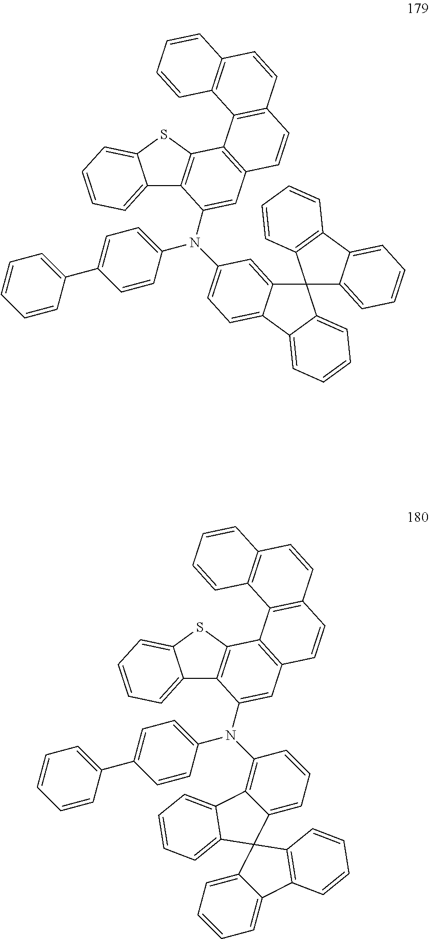 [SMILES: c1ccc(-c2ccc(N(c3ccc4c(c3)C3(c5ccccc5-c5ccccc53)c3ccccc3-4)c3cc4ccc5ccc6ccccc6c5c4c4sc5ccccc5c34)cc2)cc1 ; c1ccc(-c2ccc(N(c3cccc4c3-c3ccccc3C43c4ccccc4-c4ccccc43)c3cc4ccc5ccc6ccccc6c5c4c4sc5ccccc5c34)cc2)cc1]

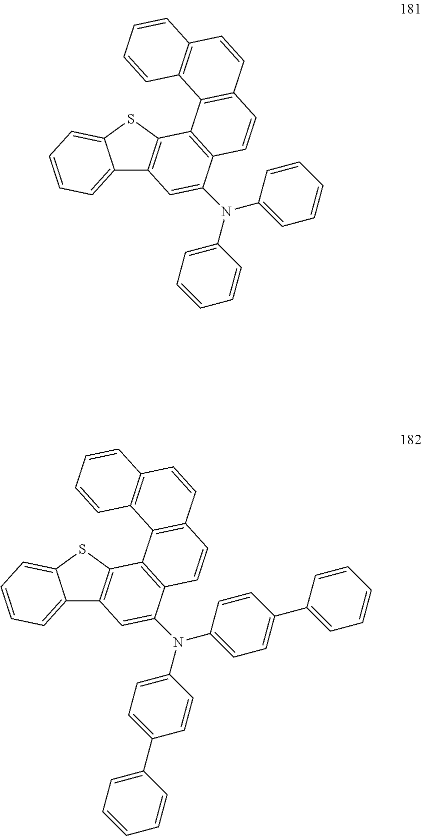 [SMILES: c1ccc(N(c2ccccc2)c2cc3c4ccccc4sc3c3c2ccc2ccc4ccccc4c23)cc1 ; c1ccc(-c2ccc(N(c3ccc(-c4ccccc4)cc3)c3cc4c5ccccc5sc4c4c3ccc3ccc5ccccc5c34)cc2)cc1]

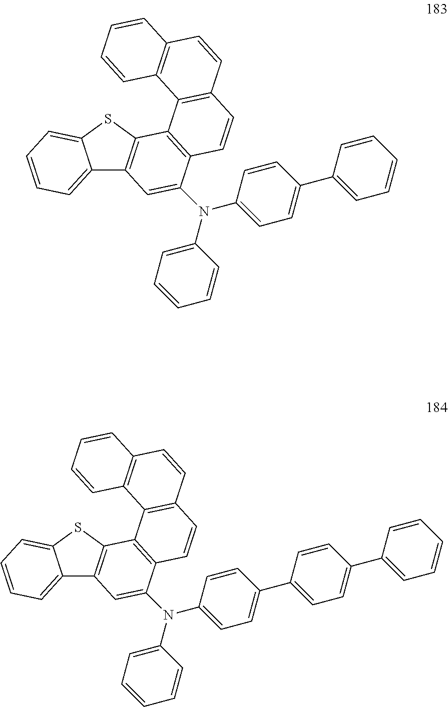 [SMILES: c1ccc(-c2ccc(N(c3ccccc3)c3cc4c5ccccc5sc4c4c3ccc3ccc5ccccc5c34)cc2)cc1 ; c1ccc(-c2ccc(-c3ccc(N(c4ccccc4)c4cc5c6ccccc6sc5c5c4ccc4ccc6ccccc6c45)cc3)cc2)cc1]

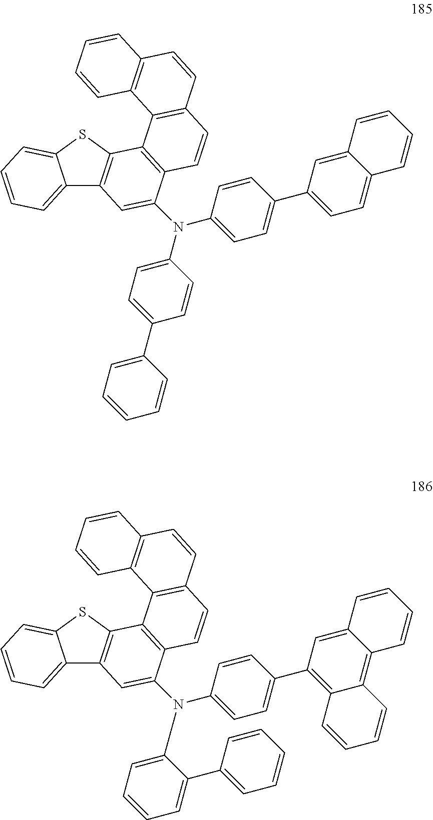 [SMILES: c1ccc(-c2ccc(N(c3ccc(-c4ccc5ccccc5c4)cc3)c3cc4c5ccccc5sc4c4c3ccc3ccc5ccccc5c34)cc2)cc1 ; c1ccc(-c2ccccc2N(c2ccc(-c3cc4ccccc4c4ccccc34)cc2)c2cc3c4ccccc4sc3c3c2ccc2ccc4ccccc4c23)cc1]

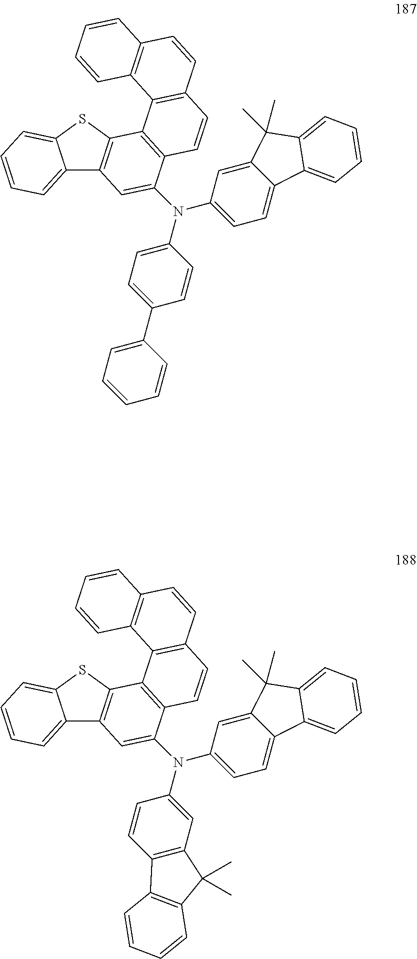 [SMILES: CC1(C)c2ccccc2-c2ccc(N(c3ccc(-c4ccccc4)cc3)c3cc4c5ccccc5sc4c4c3ccc3ccc5ccccc5c34)cc21 ; CC1(C)c2ccccc2-c2ccc(N(c3ccc4c(c3)C(C)(C)c3ccccc3-4)c3cc4c5ccccc5sc4c4c3ccc3ccc5ccccc5c34)cc21]

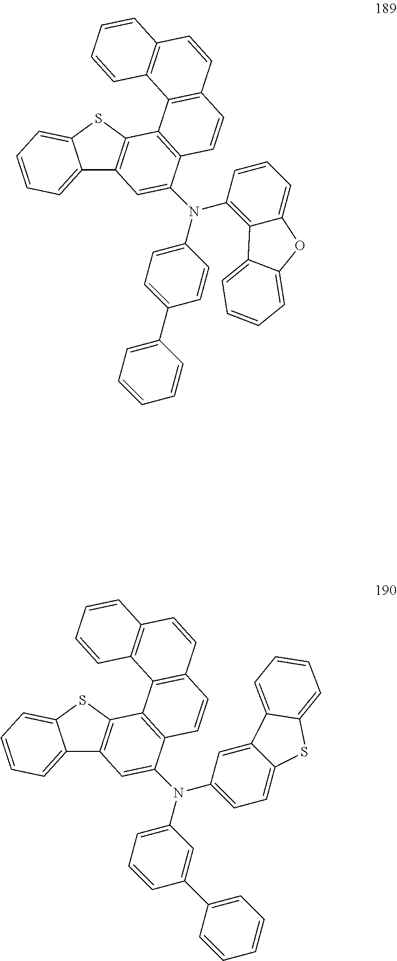 [SMILES: c1ccc(-c2ccc(N(c3cc4c5ccccc5sc4c4c3ccc3ccc5ccccc5c34)c3cccc4oc5ccccc5c34)cc2)cc1 ; c1ccc(-c2cccc(N(c3ccc4sc5ccccc5c4c3)c3cc4c5ccccc5sc4c4c3ccc3ccc5ccccc5c34)c2)cc1]

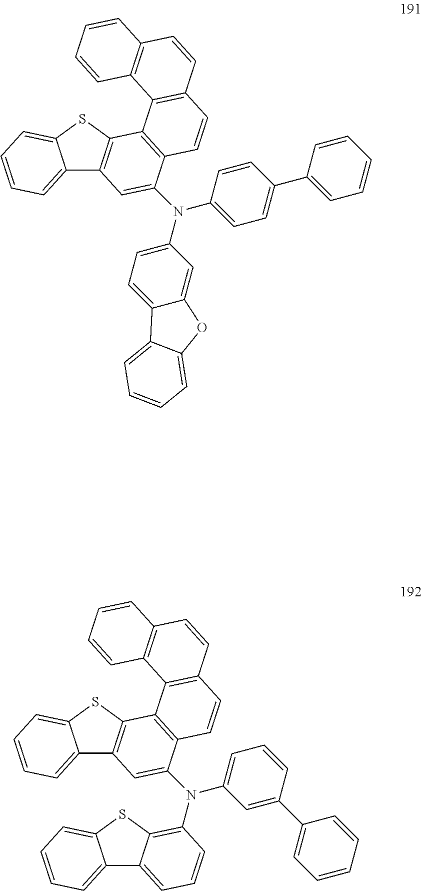 [SMILES: c1ccc(-c2ccc(N(c3ccc4c(c3)oc3ccccc34)c3cc4c5ccccc5sc4c4c3ccc3ccc5ccccc5c34)cc2)cc1 ; c1ccc(-c2cccc(N(c3cc4c5ccccc5sc4c4c3ccc3ccc5ccccc5c34)c3cccc4c3sc3ccccc34)c2)cc1]

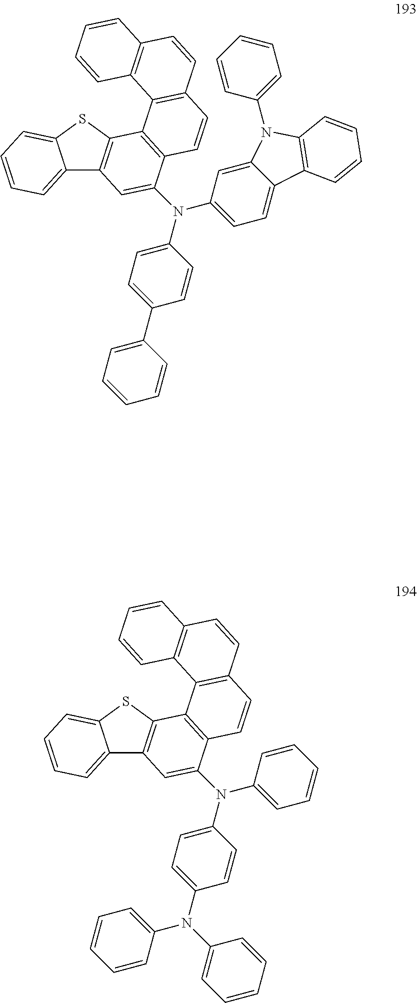 [SMILES: c1ccc(-c2ccc(N(c3ccc4c5ccccc5n(-c5ccccc5)c4c3)c3cc4c5ccccc5sc4c4c3ccc3ccc5ccccc5c34)cc2)cc1 ; c1ccc(N(c2ccccc2)c2ccc(N(c3ccccc3)c3cc4c5ccccc5sc4c4c3ccc3ccc5ccccc5c34)cc2)cc1]

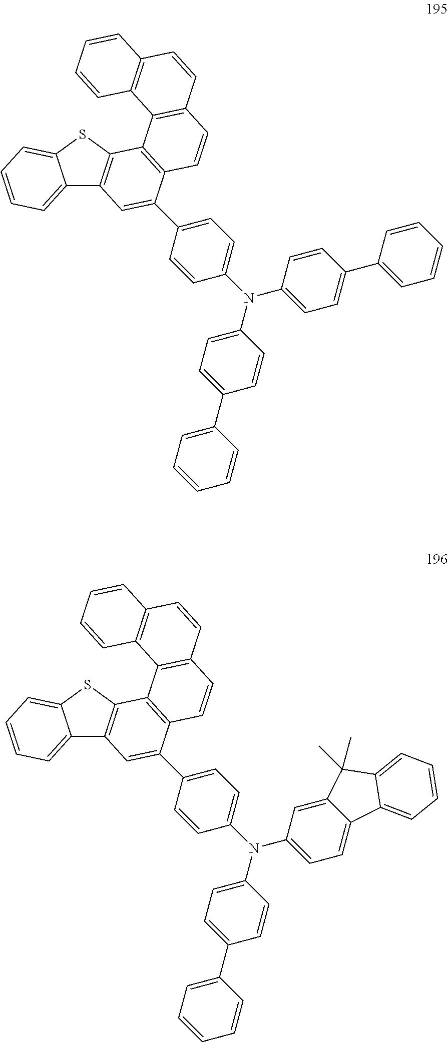 [SMILES: c1ccc(-c2ccc(N(c3ccc(-c4ccccc4)cc3)c3ccc(-c4cc5c6ccccc6sc5c5c4ccc4ccc6ccccc6c45)cc3)cc2)cc1 ; CC1(C)c2ccccc2-c2ccc(N(c3ccc(-c4ccccc4)cc3)c3ccc(-c4cc5c6ccccc6sc5c5c4ccc4ccc6ccccc6c45)cc3)cc21]

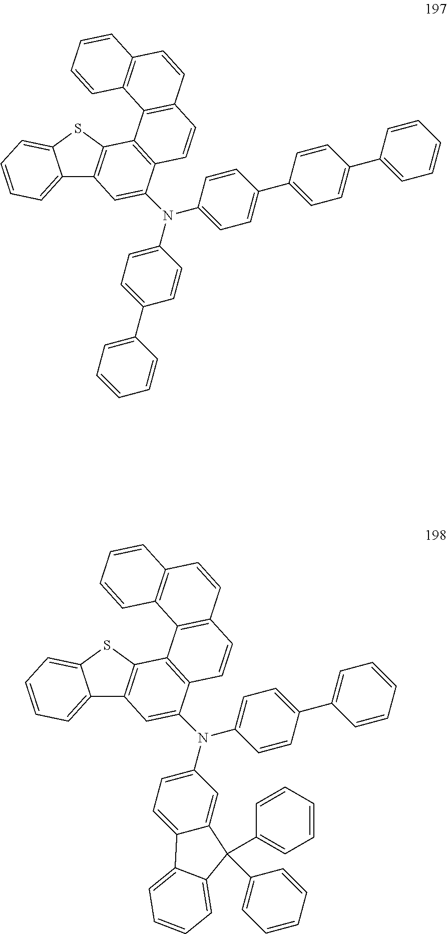 [SMILES: c1ccc(-c2ccc(-c3ccc(N(c4ccc(-c5ccccc5)cc4)c4cc5c6ccccc6sc5c5c4ccc4ccc6ccccc6c45)cc3)cc2)cc1 ; c1ccc(-c2ccc(N(c3ccc4c(c3)C(c3ccccc3)(c3ccccc3)c3ccccc3-4)c3cc4c5ccccc5sc4c4c3ccc3ccc5ccccc5c34)cc2)cc1]

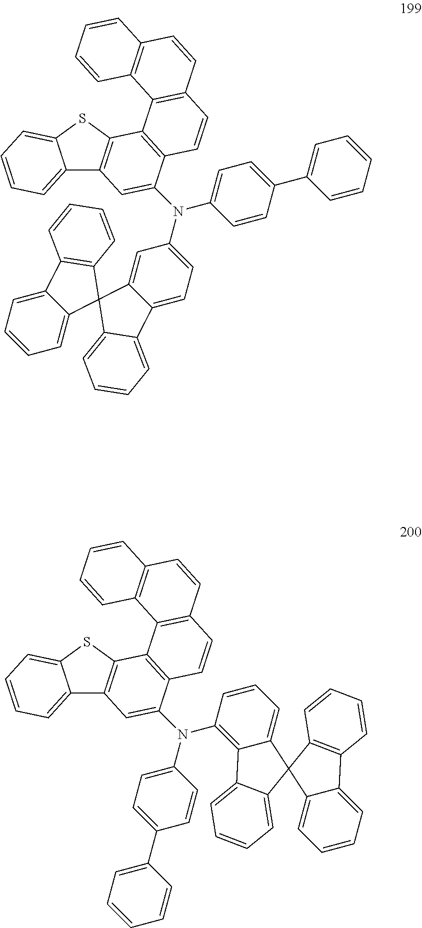 [SMILES: c1ccc(-c2ccc(N(c3ccc4c(c3)C3(c5ccccc5-c5ccccc53)c3ccccc3-4)c3cc4c5ccccc5sc4c4c3ccc3ccc5ccccc5c34)cc2)cc1 ; c1ccc(-c2ccc(N(c3cccc4c3-c3ccccc3C43c4ccccc4-c4ccccc43)c3cc4c5ccccc5sc4c4c3ccc3ccc5ccccc5c34)cc2)cc1]

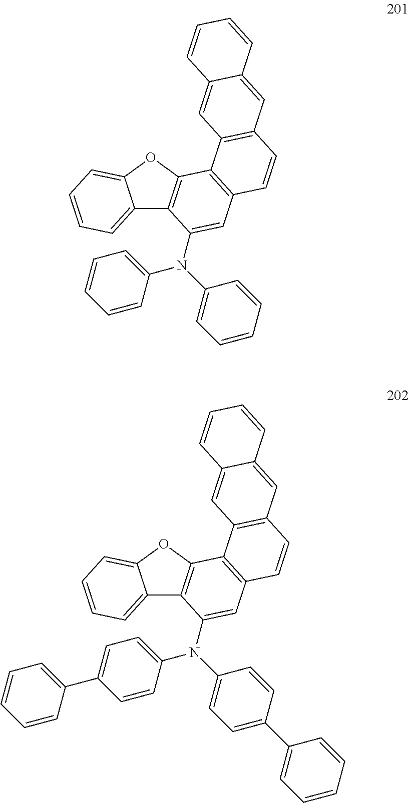 [SMILES: c1ccc(N(c2ccccc2)c2cc3ccc4cc5ccccc5cc4c3c3oc4ccccc4c23)cc1 ; c1ccc(-c2ccc(N(c3ccc(-c4ccccc4)cc3)c3cc4ccc5cc6ccccc6cc5c4c4oc5ccccc5c34)cc2)cc1]

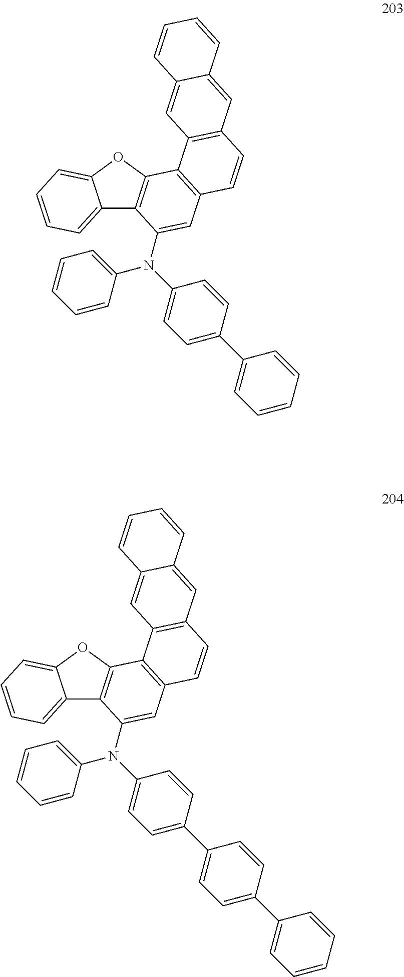 [SMILES: c1ccc(-c2ccc(N(c3ccccc3)c3cc4ccc5cc6ccccc6cc5c4c4oc5ccccc5c34)cc2)cc1 ; c1ccc(-c2ccc(-c3ccc(N(c4ccccc4)c4cc5ccc6cc7ccccc7cc6c5c5oc6ccccc6c45)cc3)cc2)cc1]

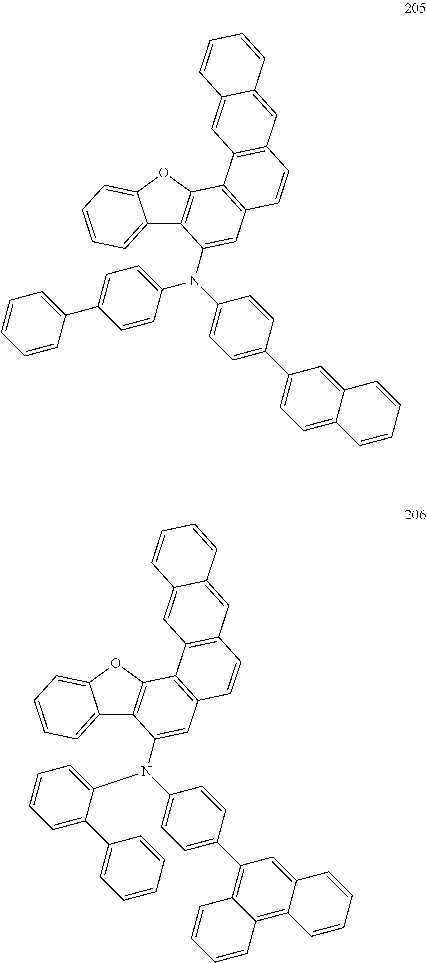 [SMILES: c1ccc(-c2ccc(N(c3ccc(-c4ccc5ccccc5c4)cc3)c3cc4ccc5cc6ccccc6cc5c4c4oc5ccccc5c34)cc2)cc1 ; c1ccc(-c2ccccc2N(c2ccc(-c3cc4ccccc4c4ccccc34)cc2)c2cc3ccc4cc5ccccc5cc4c3c3oc4ccccc4c23)cc1]

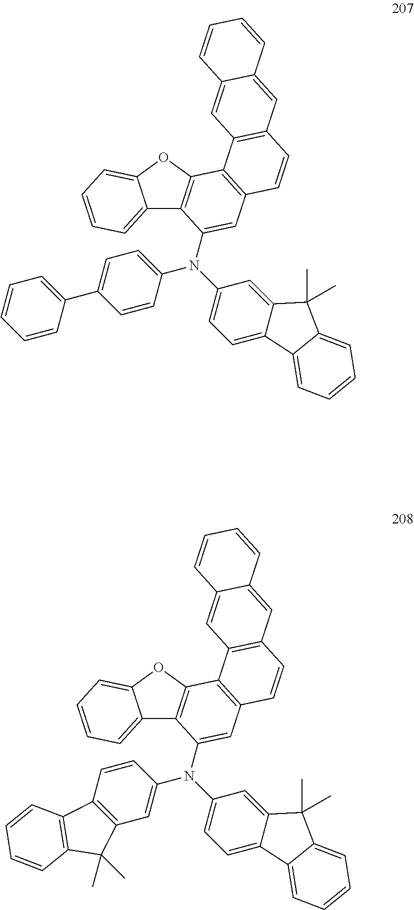 [SMILES: CC1(C)c2ccccc2-c2ccc(N(c3ccc(-c4ccccc4)cc3)c3cc4ccc5cc6ccccc6cc5c4c4oc5ccccc5c34)cc21 ; CC1(C)c2ccccc2-c2ccc(N(c3ccc4c(c3)C(C)(C)c3ccccc3-4)c3cc4ccc5cc6ccccc6cc5c4c4oc5ccccc5c34)cc21]

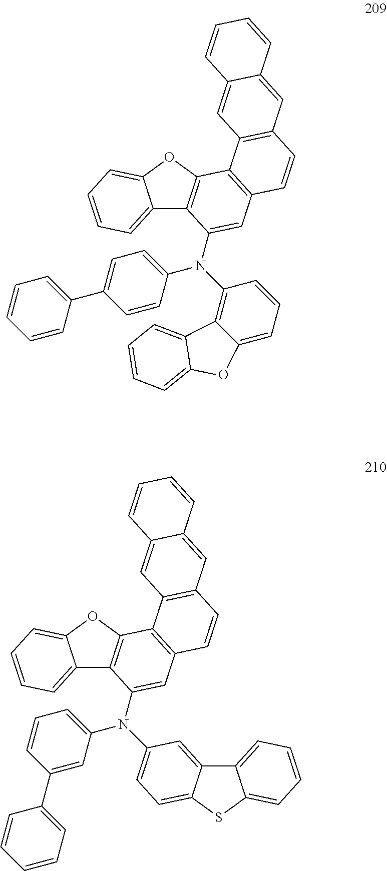 [SMILES: c1ccc(-c2ccc(N(c3cccc4oc5ccccc5c34)c3cc4ccc5cc6ccccc6cc5c4c4oc5ccccc5c34)cc2)cc1 ; c1ccc(-c2cccc(N(c3ccc4sc5ccccc5c4c3)c3cc4ccc5cc6ccccc6cc5c4c4oc5ccccc5c34)c2)cc1]

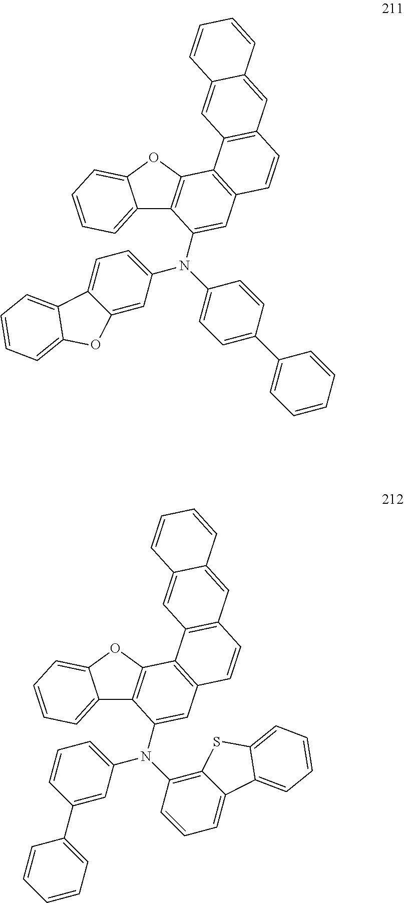 [SMILES: c1ccc(-c2ccc(N(c3ccc4c(c3)oc3ccccc34)c3cc4ccc5cc6ccccc6cc5c4c4oc5ccccc5c34)cc2)cc1 ; c1ccc(-c2cccc(N(c3cccc4c3sc3ccccc34)c3cc4ccc5cc6ccccc6cc5c4c4oc5ccccc5c34)c2)cc1]

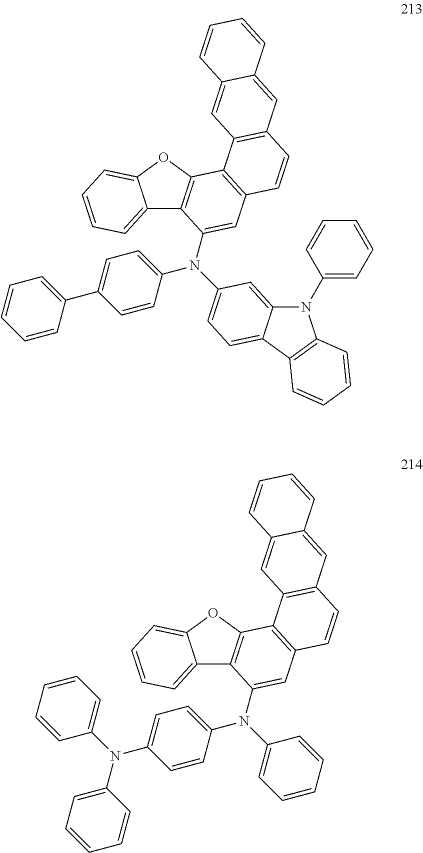 [SMILES: c1ccc(-c2ccc(N(c3ccc4c5ccccc5n(-c5ccccc5)c4c3)c3cc4ccc5cc6ccccc6cc5c4c4oc5ccccc5c34)cc2)cc1 ; c1ccc(N(c2ccccc2)c2ccc(N(c3ccccc3)c3cc4ccc5cc6ccccc6cc5c4c4oc5ccccc5c34)cc2)cc1]

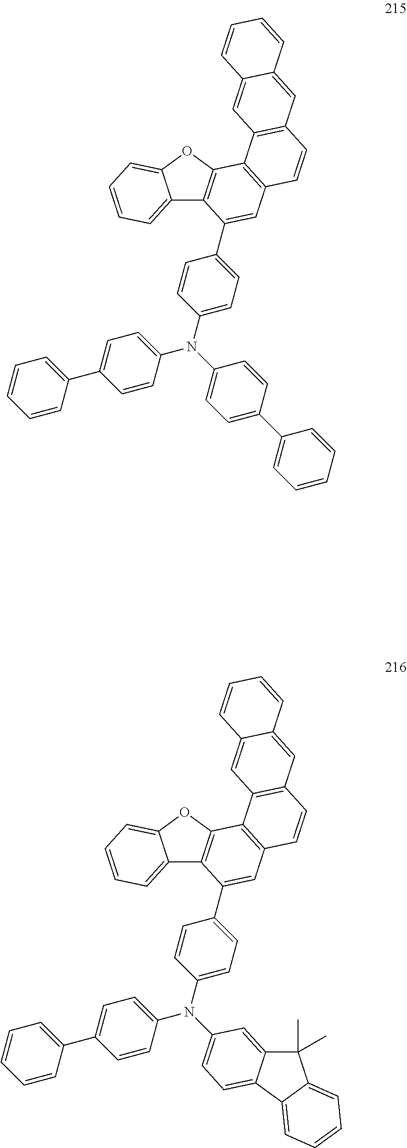 [SMILES: c1ccc(-c2ccc(N(c3ccc(-c4ccccc4)cc3)c3ccc(-c4cc5ccc6cc7ccccc7cc6c5c5oc6ccccc6c45)cc3)cc2)cc1 ; CC1(C)c2ccccc2-c2ccc(N(c3ccc(-c4ccccc4)cc3)c3ccc(-c4cc5ccc6cc7ccccc7cc6c5c5oc6ccccc6c45)cc3)cc21]

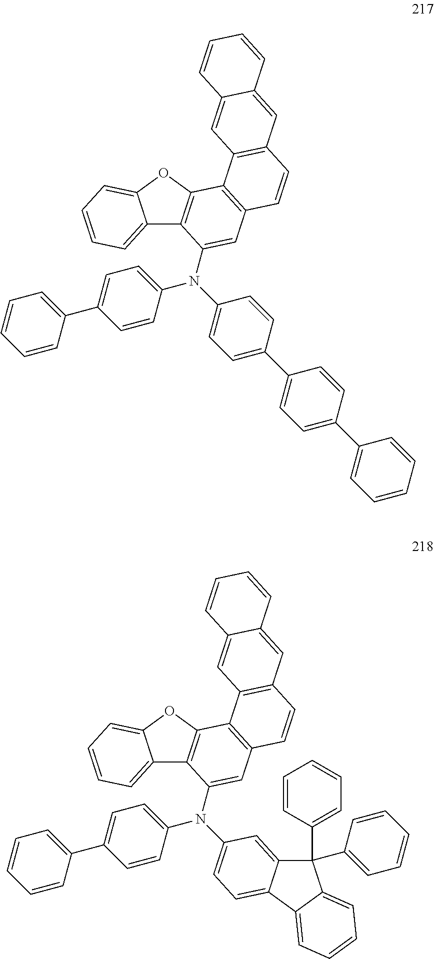 [SMILES: c1ccc(-c2ccc(-c3ccc(N(c4ccc(-c5ccccc5)cc4)c4cc5ccc6cc7ccccc7cc6c5c5oc6ccccc6c45)cc3)cc2)cc1 ; c1ccc(-c2ccc(N(c3ccc4c(c3)C(c3ccccc3)(c3ccccc3)c3ccccc3-4)c3cc4ccc5cc6ccccc6cc5c4c4oc5ccccc5c34)cc2)cc1]

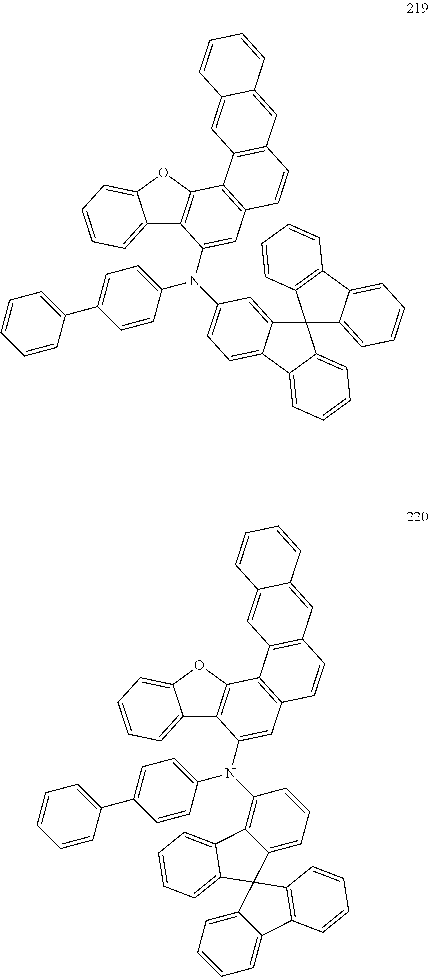 [SMILES: c1ccc(-c2ccc(N(c3ccc4c(c3)C3(c5ccccc5-c5ccccc53)c3ccccc3-4)c3cc4ccc5cc6ccccc6cc5c4c4oc5ccccc5c34)cc2)cc1 ; c1ccc(-c2ccc(N(c3cccc4c3-c3ccccc3C43c4ccccc4-c4ccccc43)c3cc4ccc5cc6ccccc6cc5c4c4oc5ccccc5c34)cc2)cc1]

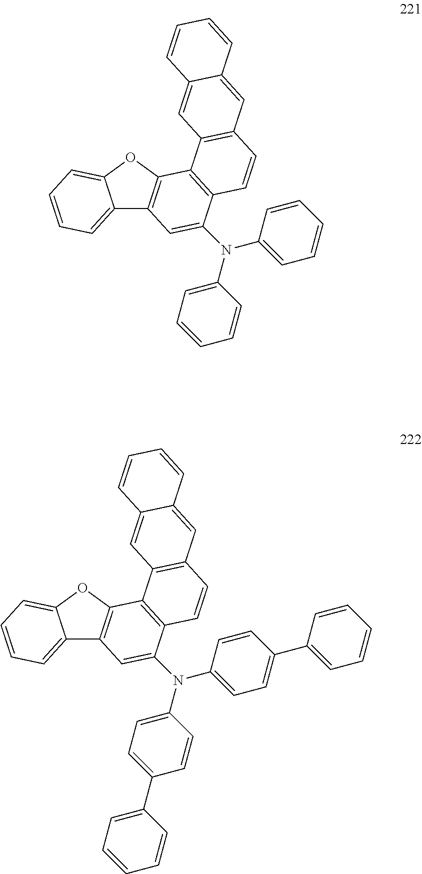 [SMILES: c1ccc(N(c2ccccc2)c2cc3c4ccccc4oc3c3c2ccc2cc4ccccc4cc23)cc1 ; c1ccc(-c2ccc(N(c3ccc(-c4ccccc4)cc3)c3cc4c5ccccc5oc4c4c3ccc3cc5ccccc5cc34)cc2)cc1]

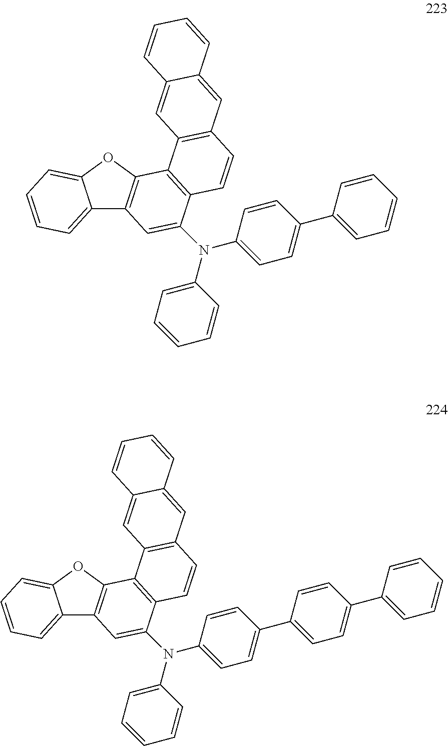 [SMILES: c1ccc(-c2ccc(N(c3ccccc3)c3cc4c5ccccc5oc4c4c3ccc3cc5ccccc5cc34)cc2)cc1 ; c1ccc(-c2ccc(-c3ccc(N(c4ccccc4)c4cc5c6ccccc6oc5c5c4ccc4cc6ccccc6cc45)cc3)cc2)cc1]

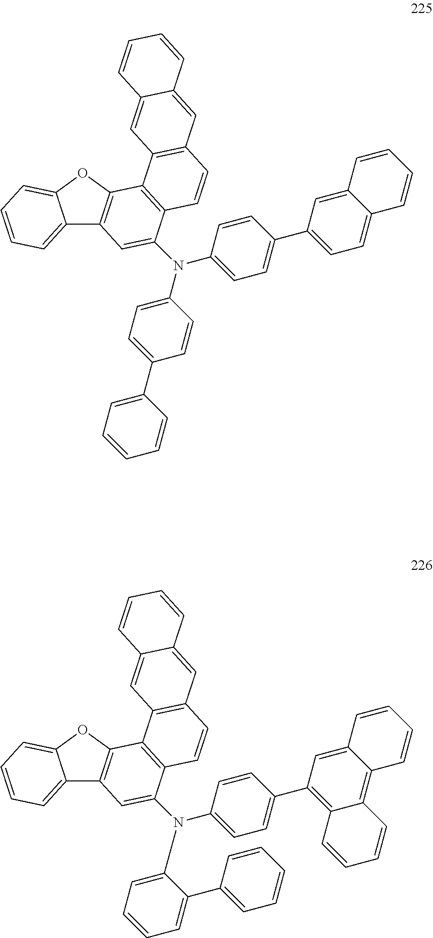 [SMILES: c1ccc(-c2ccc(N(c3ccc(-c4ccc5ccccc5c4)cc3)c3cc4c5ccccc5oc4c4c3ccc3cc5ccccc5cc34)cc2)cc1 ; c1ccc(-c2ccccc2N(c2ccc(-c3cc4ccccc4c4ccccc34)cc2)c2cc3c4ccccc4oc3c3c2ccc2cc4ccccc4cc23)cc1]

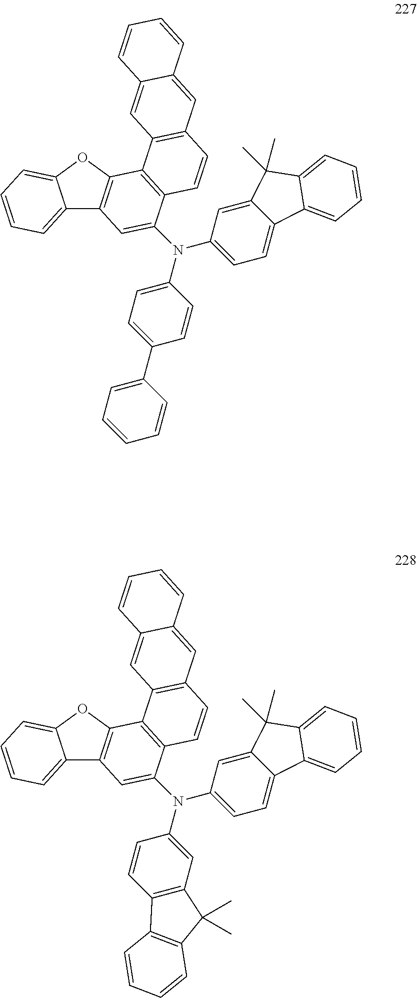 [SMILES: CC1(C)c2ccccc2-c2ccc(N(c3ccc(-c4ccccc4)cc3)c3cc4c5ccccc5oc4c4c3ccc3cc5ccccc5cc34)cc21 ; CC1(C)c2ccccc2-c2ccc(N(c3ccc4c(c3)C(C)(C)c3ccccc3-4)c3cc4c5ccccc5oc4c4c3ccc3cc5ccccc5cc34)cc21]

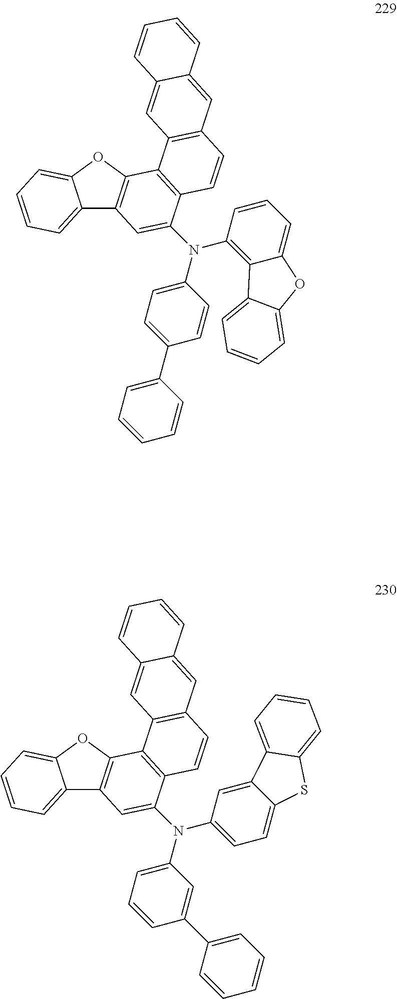 [SMILES: c1ccc(-c2ccc(N(c3cc4c5ccccc5oc4c4c3ccc3cc5ccccc5cc34)c3cccc4oc5ccccc5c34)cc2)cc1 ; c1ccc(-c2cccc(N(c3ccc4sc5ccccc5c4c3)c3cc4c5ccccc5oc4c4c3ccc3cc5ccccc5cc34)c2)cc1]

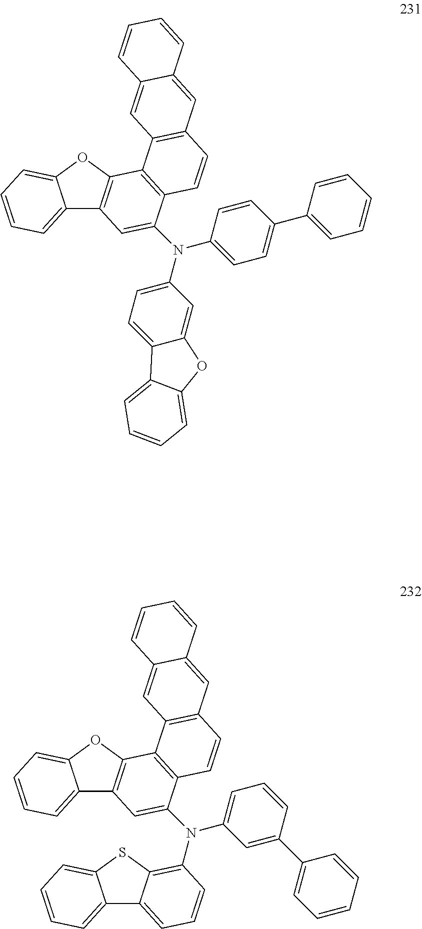 [SMILES: c1ccc(-c2ccc(N(c3ccc4c(c3)oc3ccccc34)c3cc4c5ccccc5oc4c4c3ccc3cc5ccccc5cc34)cc2)cc1 ; c1ccc(-c2cccc(N(c3cc4c5ccccc5oc4c4c3ccc3cc5ccccc5cc34)c3cccc4c3sc3ccccc34)c2)cc1]

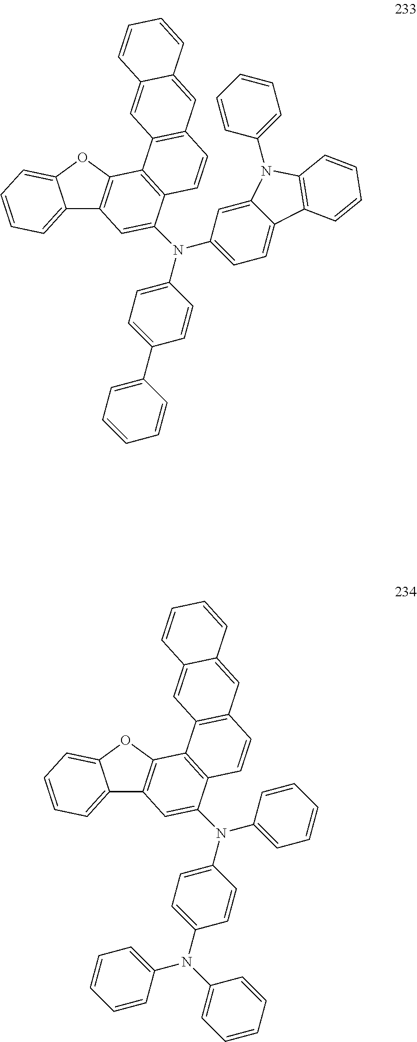 [SMILES: c1ccc(-c2ccc(N(c3ccc4c5ccccc5n(-c5ccccc5)c4c3)c3cc4c5ccccc5oc4c4c3ccc3cc5ccccc5cc34)cc2)cc1 ; c1ccc(N(c2ccccc2)c2ccc(N(c3ccccc3)c3cc4c5ccccc5oc4c4c3ccc3cc5ccccc5cc34)cc2)cc1]

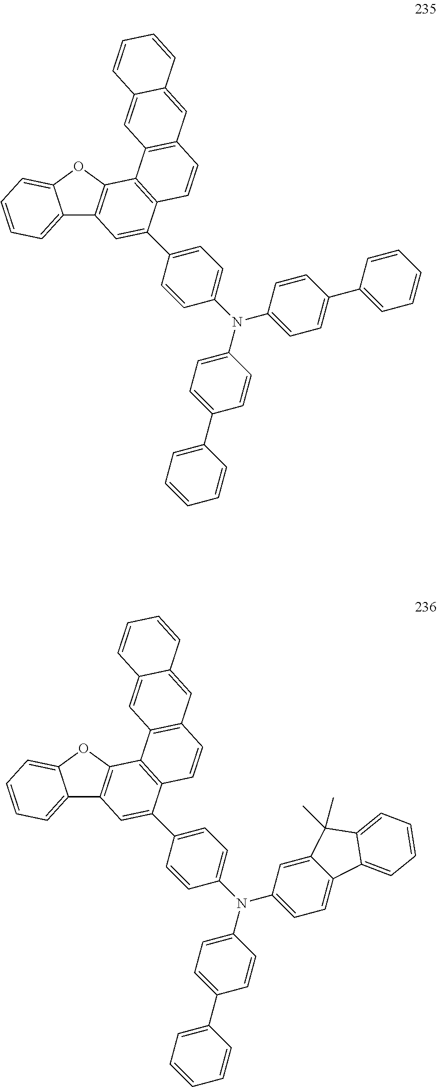 [SMILES: c1ccc(-c2ccc(N(c3ccc(-c4ccccc4)cc3)c3ccc(-c4cc5c6ccccc6oc5c5c4ccc4cc6ccccc6cc45)cc3)cc2)cc1 ; CC1(C)c2ccccc2-c2ccc(N(c3ccc(-c4ccccc4)cc3)c3ccc(-c4cc5c6ccccc6oc5c5c4ccc4cc6ccccc6cc45)cc3)cc21]

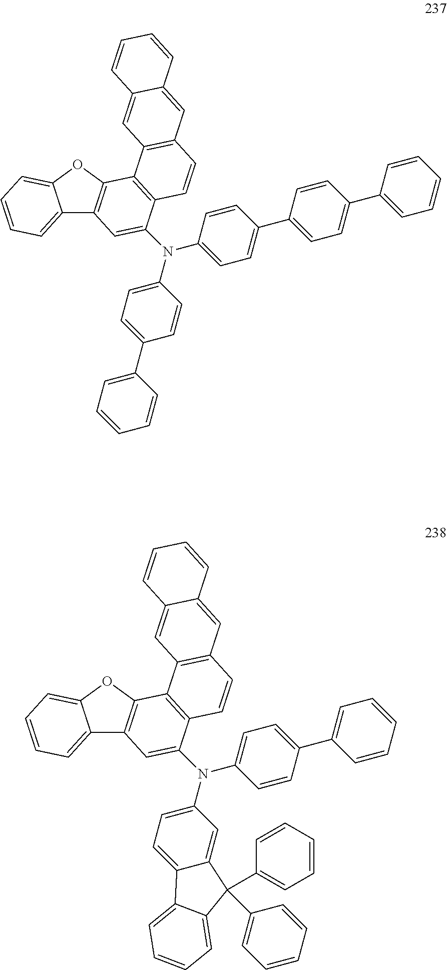 [SMILES: c1ccc(-c2ccc(-c3ccc(N(c4ccc(-c5ccccc5)cc4)c4cc5c6ccccc6oc5c5c4ccc4cc6ccccc6cc45)cc3)cc2)cc1 ; c1ccc(-c2ccc(N(c3ccc4c(c3)C(c3ccccc3)(c3ccccc3)c3ccccc3-4)c3cc4c5ccccc5oc4c4c3ccc3cc5ccccc5cc34)cc2)cc1]

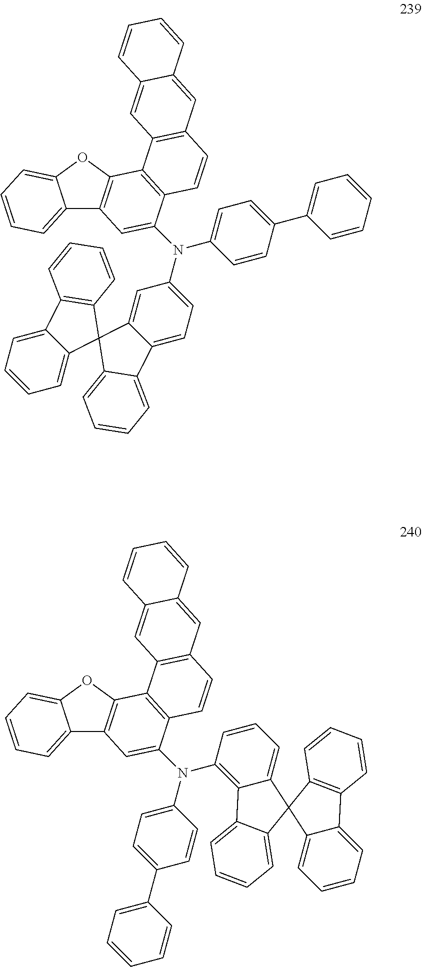 [SMILES: c1ccc(-c2ccc(N(c3ccc4c(c3)C3(c5ccccc5-c5ccccc53)c3ccccc3-4)c3cc4c5ccccc5oc4c4c3ccc3cc5ccccc5cc34)cc2)cc1 ; c1ccc(-c2ccc(N(c3cccc4c3-c3ccccc3C43c4ccccc4-c4ccccc43)c3cc4c5ccccc5oc4c4c3ccc3cc5ccccc5cc34)cc2)cc1]

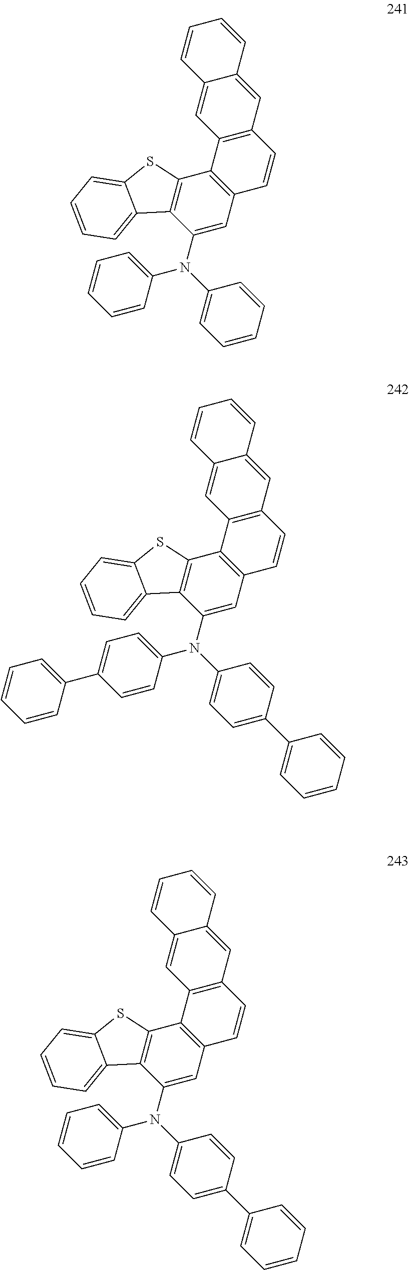 [SMILES: c1ccc(N(c2ccccc2)c2cc3ccc4cc5ccccc5cc4c3c3sc4ccccc4c23)cc1 ; c1ccc(-c2ccc(N(c3ccc(-c4ccccc4)cc3)c3cc4ccc5cc6ccccc6cc5c4c4sc5ccccc5c34)cc2)cc1 ; c1ccc(-c2ccc(N(c3ccccc3)c3cc4ccc5cc6ccccc6cc5c4c4sc5ccccc5c34)cc2)cc1]

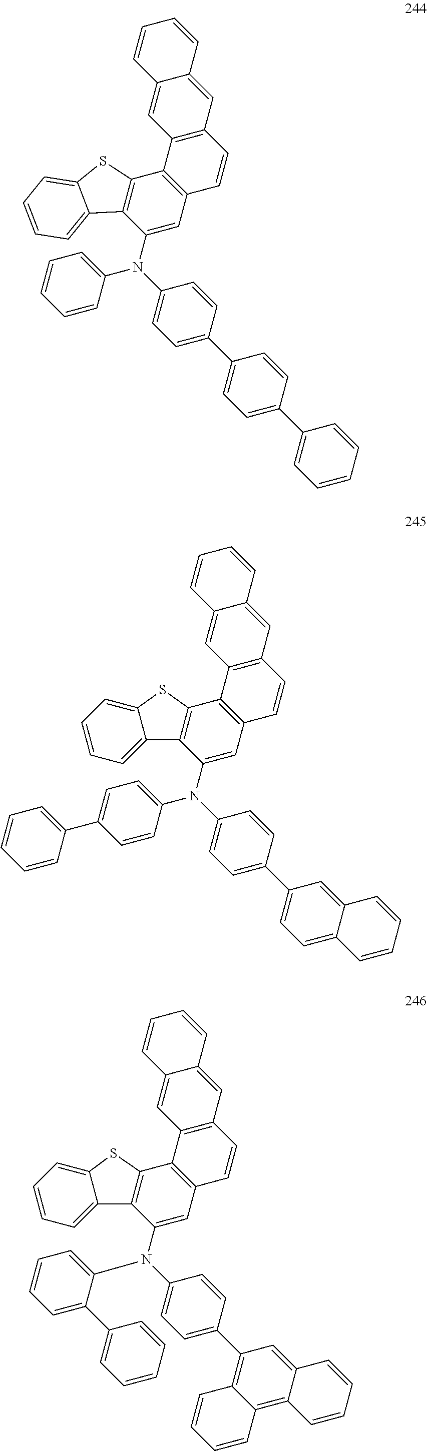 [SMILES: c1ccc(-c2ccc(-c3ccc(N(c4ccccc4)c4cc5ccc6cc7ccccc7cc6c5c5sc6ccccc6c45)cc3)cc2)cc1 ; c1ccc(-c2ccc(N(c3ccc(-c4ccc5ccccc5c4)cc3)c3cc4ccc5cc6ccccc6cc5c4c4sc5ccccc5c34)cc2)cc1 ; c1ccc(-c2ccccc2N(c2ccc(-c3cc4ccccc4c4ccccc34)cc2)c2cc3ccc4cc5ccccc5cc4c3c3sc4ccccc4c23)cc1]

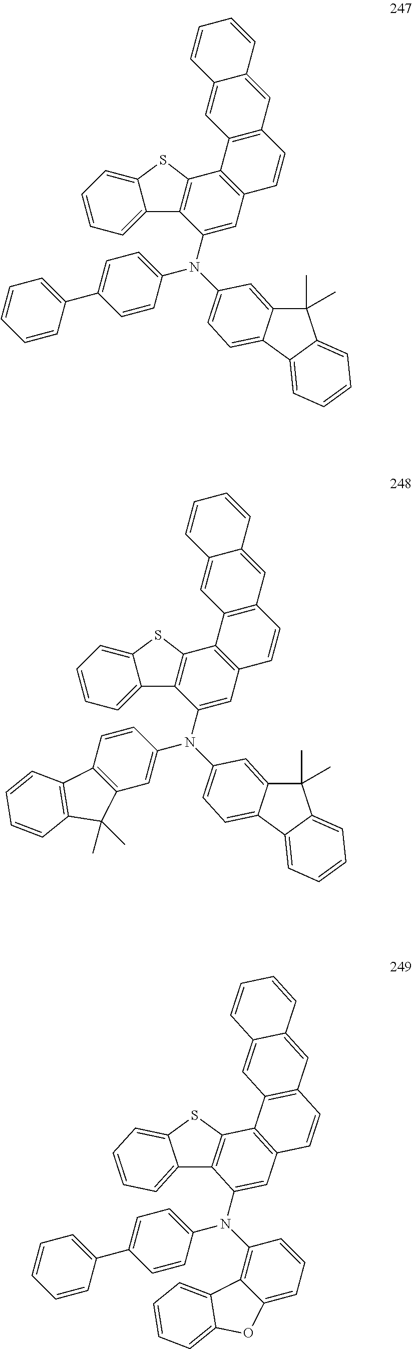 [SMILES: CC1(C)c2ccccc2-c2ccc(N(c3ccc(-c4ccccc4)cc3)c3cc4ccc5cc6ccccc6cc5c4c4sc5ccccc5c34)cc21 ; CC1(C)c2ccccc2-c2ccc(N(c3ccc4c(c3)C(C)(C)c3ccccc3-4)c3cc4ccc5cc6ccccc6cc5c4c4sc5ccccc5c34)cc21 ; c1ccc(-c2ccc(N(c3cccc4oc5ccccc5c34)c3cc4ccc5cc6ccccc6cc5c4c4sc5ccccc5c34)cc2)cc1]

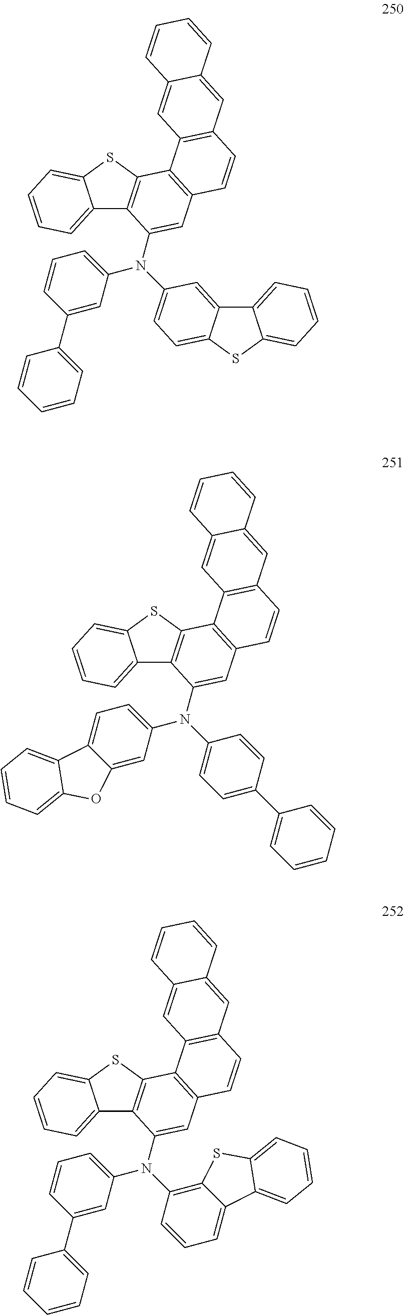 [SMILES: c1ccc(-c2cccc(N(c3ccc4sc5ccccc5c4c3)c3cc4ccc5cc6ccccc6cc5c4c4sc5ccccc5c34)c2)cc1 ; c1ccc(-c2ccc(N(c3ccc4c(c3)oc3ccccc34)c3cc4ccc5cc6ccccc6cc5c4c4sc5ccccc5c34)cc2)cc1 ; c1ccc(-c2cccc(N(c3cccc4c3sc3ccccc34)c3cc4ccc5cc6ccccc6cc5c4c4sc5ccccc5c34)c2)cc1]

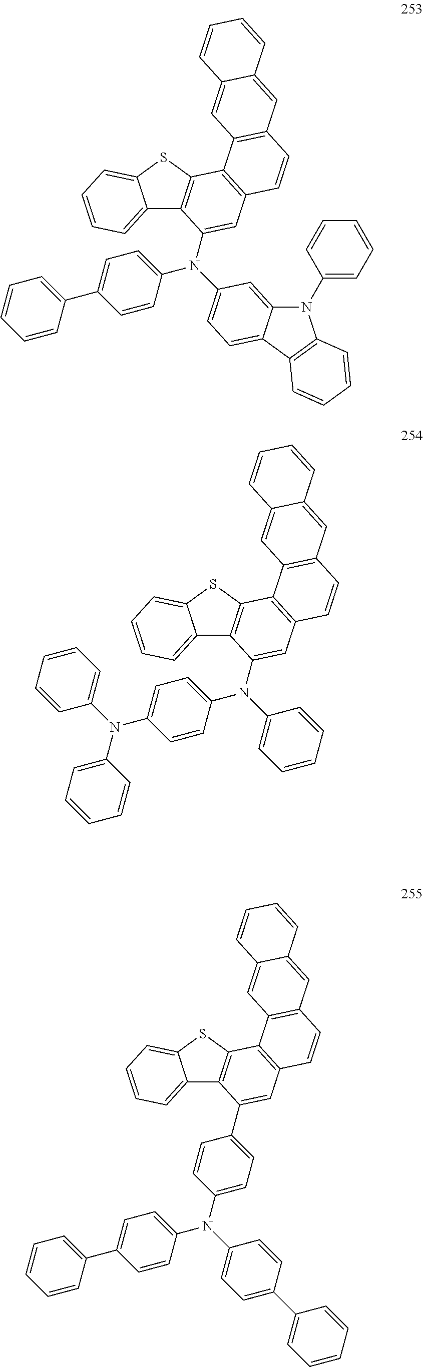 [SMILES: c1ccc(-c2ccc(N(c3ccc4c5ccccc5n(-c5ccccc5)c4c3)c3cc4ccc5cc6ccccc6cc5c4c4sc5ccccc5c34)cc2)cc1 ; c1ccc(N(c2ccccc2)c2ccc(N(c3ccccc3)c3cc4ccc5cc6ccccc6cc5c4c4sc5ccccc5c34)cc2)cc1 ; c1ccc(-c2ccc(N(c3ccc(-c4ccccc4)cc3)c3ccc(-c4cc5ccc6cc7ccccc7cc6c5c5sc6ccccc6c45)cc3)cc2)cc1]

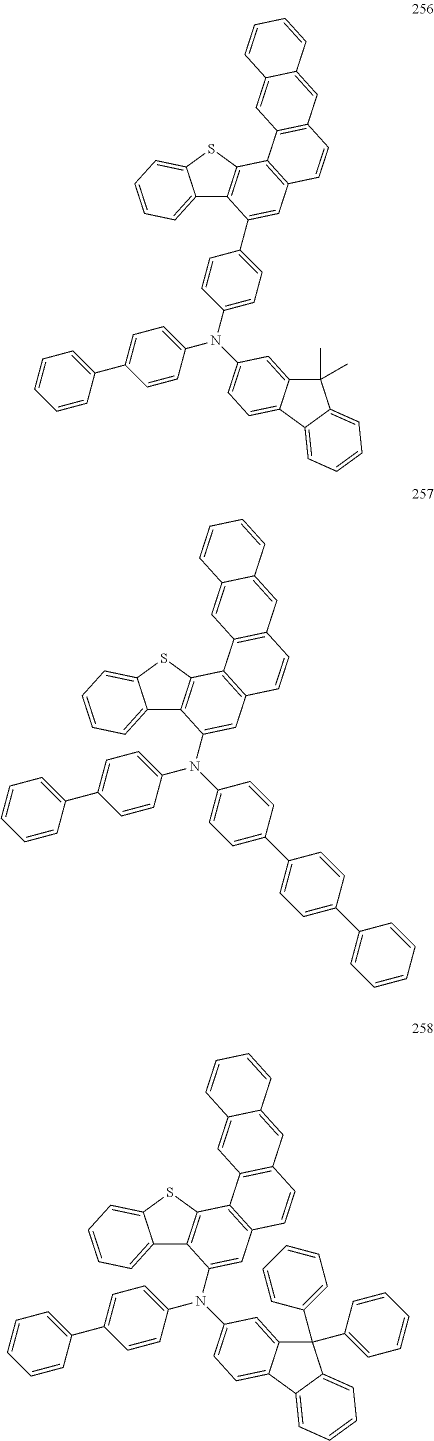 [SMILES: CC1(C)c2ccccc2-c2ccc(N(c3ccc(-c4ccccc4)cc3)c3ccc(-c4cc5ccc6cc7ccccc7cc6c5c5sc6ccccc6c45)cc3)cc21 ; c1ccc(-c2ccc(-c3ccc(N(c4ccc(-c5ccccc5)cc4)c4cc5ccc6cc7ccccc7cc6c5c5sc6ccccc6c45)cc3)cc2)cc1 ; c1ccc(-c2ccc(N(c3ccc4c(c3)C(c3ccccc3)(c3ccccc3)c3ccccc3-4)c3cc4ccc5cc6ccccc6cc5c4c4sc5ccccc5c34)cc2)cc1]

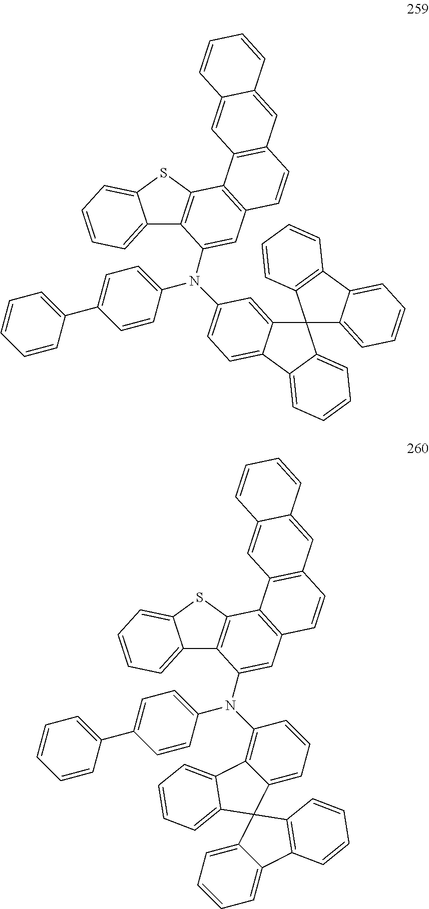 [SMILES: c1ccc(-c2ccc(N(c3ccc4c(c3)C3(c5ccccc5-c5ccccc53)c3ccccc3-4)c3cc4ccc5cc6ccccc6cc5c4c4sc5ccccc5c34)cc2)cc1 ; c1ccc(-c2ccc(N(c3cccc4c3-c3ccccc3C43c4ccccc4-c4ccccc43)c3cc4ccc5cc6ccccc6cc5c4c4sc5ccccc5c34)cc2)cc1]

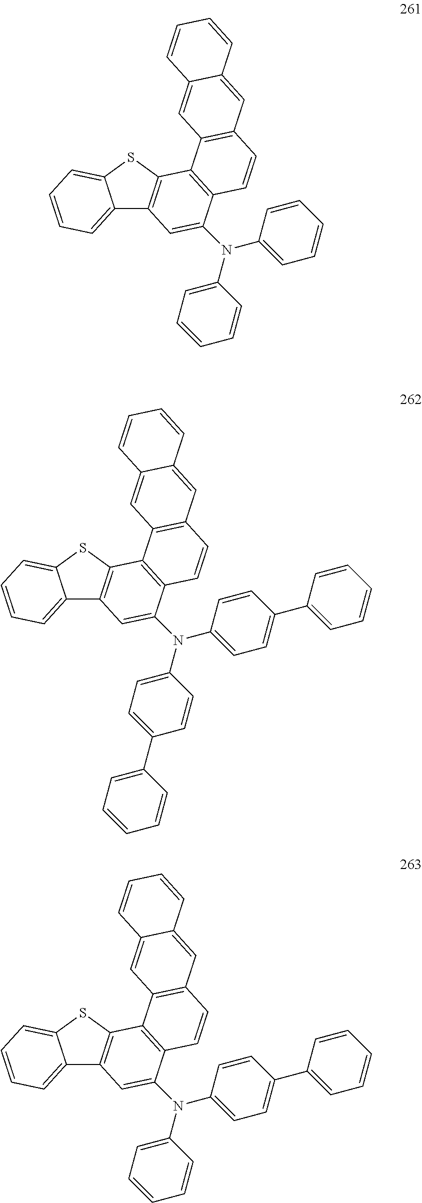 [SMILES: c1ccc(N(c2ccccc2)c2cc3c4ccccc4sc3c3c2ccc2cc4ccccc4cc23)cc1 ; c1ccc(-c2ccc(N(c3ccc(-c4ccccc4)cc3)c3cc4c5ccccc5sc4c4c3ccc3cc5ccccc5cc34)cc2)cc1 ; c1ccc(-c2ccc(N(c3ccccc3)c3cc4c5ccccc5sc4c4c3ccc3cc5ccccc5cc34)cc2)cc1]

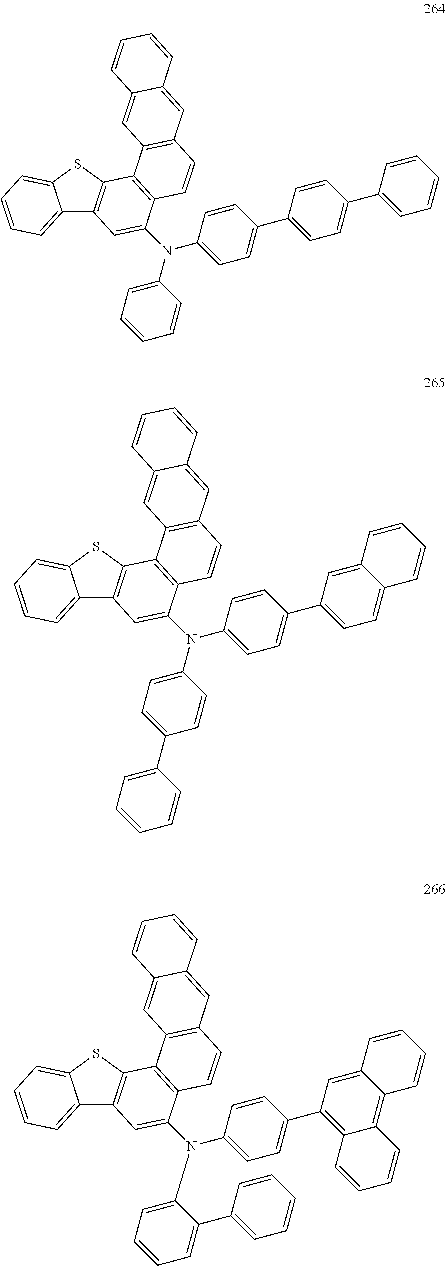 [SMILES: c1ccc(-c2ccc(-c3ccc(N(c4ccccc4)c4cc5c6ccccc6sc5c5c4ccc4cc6ccccc6cc45)cc3)cc2)cc1 ; c1ccc(-c2ccc(N(c3ccc(-c4ccc5ccccc5c4)cc3)c3cc4c5ccccc5sc4c4c3ccc3cc5ccccc5cc34)cc2)cc1 ; c1ccc(-c2ccccc2N(c2ccc(-c3cc4ccccc4c4ccccc34)cc2)c2cc3c4ccccc4sc3c3c2ccc2cc4ccccc4cc23)cc1]

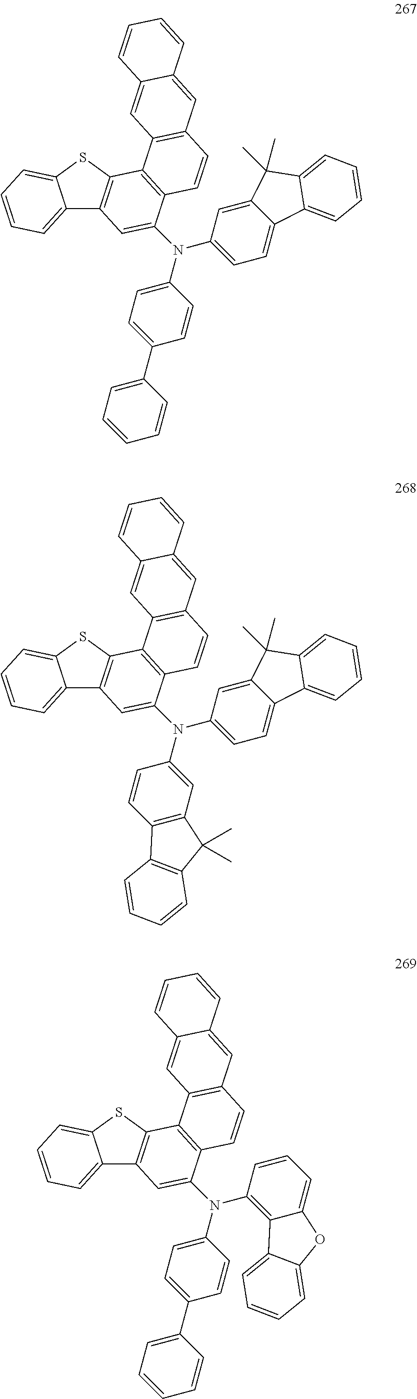 [SMILES: CC1(C)c2ccccc2-c2ccc(N(c3ccc(-c4ccccc4)cc3)c3cc4c5ccccc5sc4c4c3ccc3cc5ccccc5cc34)cc21 ; CC1(C)c2ccccc2-c2ccc(N(c3ccc4c(c3)C(C)(C)c3ccccc3-4)c3cc4c5ccccc5sc4c4c3ccc3cc5ccccc5cc34)cc21 ; c1ccc(-c2ccc(N(c3cc4c5ccccc5sc4c4c3ccc3cc5ccccc5cc34)c3cccc4oc5ccccc5c34)cc2)cc1]

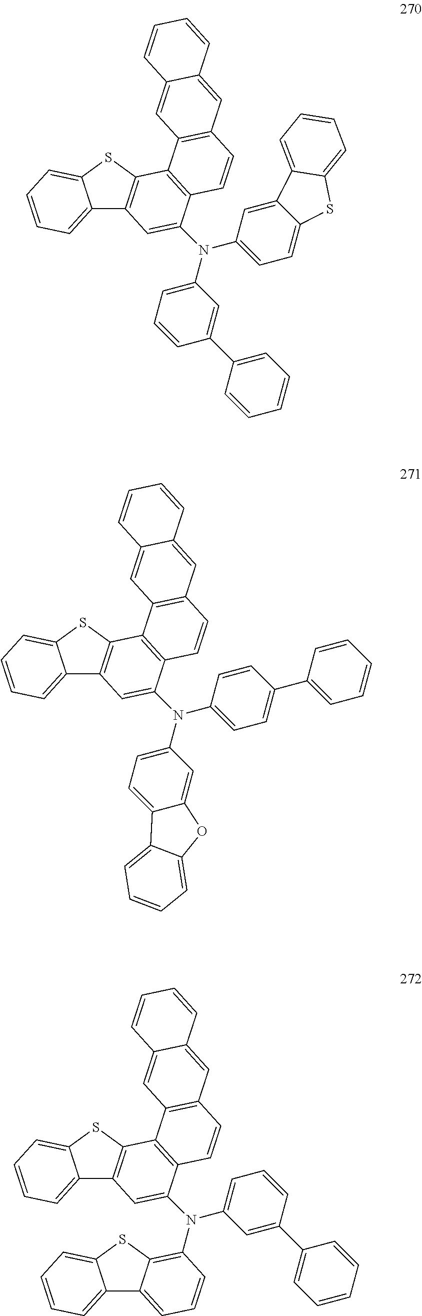 [SMILES: c1ccc(-c2cccc(N(c3ccc4sc5ccccc5c4c3)c3cc4c5ccccc5sc4c4c3ccc3cc5ccccc5cc34)c2)cc1 ; c1ccc(-c2ccc(N(c3ccc4c(c3)oc3ccccc34)c3cc4c5ccccc5sc4c4c3ccc3cc5ccccc5cc34)cc2)cc1 ; c1ccc(-c2cccc(N(c3cc4c5ccccc5sc4c4c3ccc3cc5ccccc5cc34)c3cccc4c3sc3ccccc34)c2)cc1]

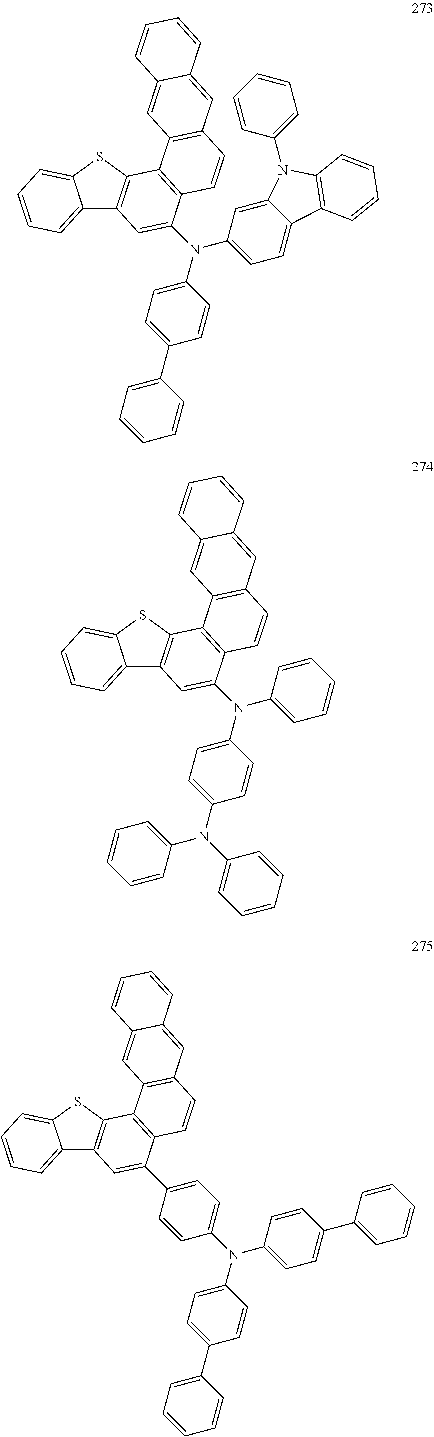 [SMILES: c1ccc(-c2ccc(N(c3ccc4c5ccccc5n(-c5ccccc5)c4c3)c3cc4c5ccccc5sc4c4c3ccc3cc5ccccc5cc34)cc2)cc1 ; c1ccc(N(c2ccccc2)c2ccc(N(c3ccccc3)c3cc4c5ccccc5sc4c4c3ccc3cc5ccccc5cc34)cc2)cc1 ; c1ccc(-c2ccc(N(c3ccc(-c4ccccc4)cc3)c3ccc(-c4cc5c6ccccc6sc5c5c4ccc4cc6ccccc6cc45)cc3)cc2)cc1]

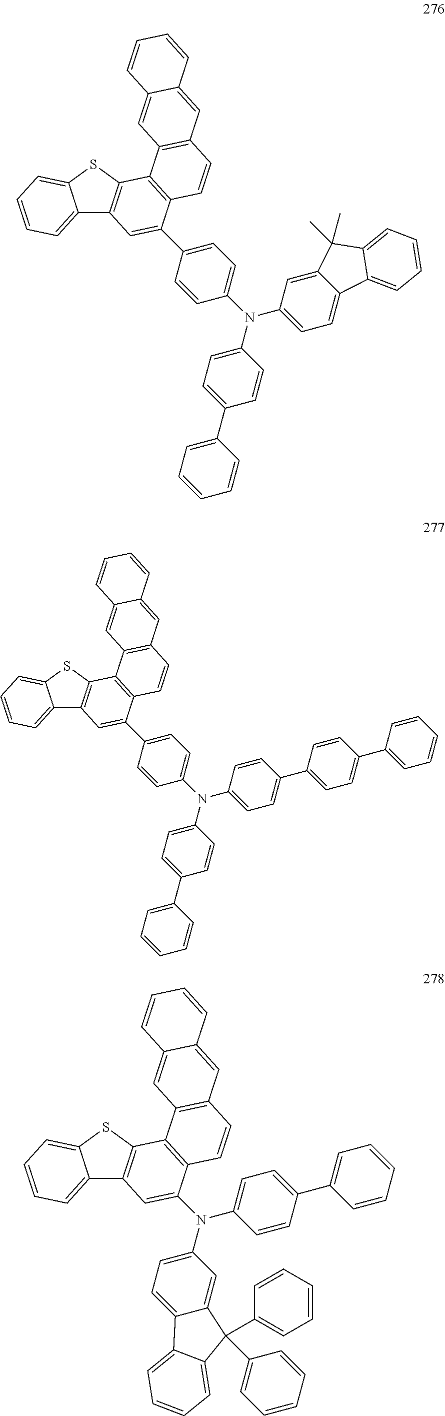

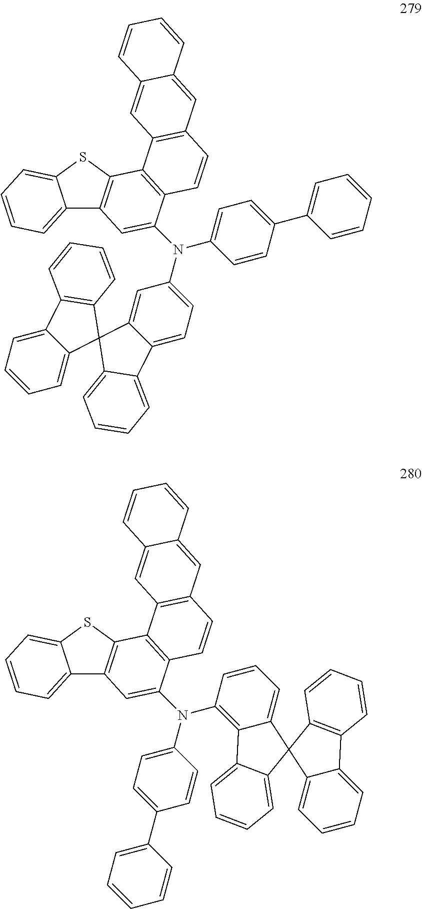 [SMILES: c1ccc(-c2ccc(N(c3ccc4c(c3)C3(c5ccccc5-c5ccccc53)c3ccccc3-4)c3cc4c5ccccc5sc4c4c3ccc3cc5ccccc5cc34)cc2)cc1 ; c1ccc(-c2ccc(N(c3cccc4c3-c3ccccc3C43c4ccccc4-c4ccccc43)c3cc4c5ccccc5sc4c4c3ccc3cc5ccccc5cc34)cc2)cc1]

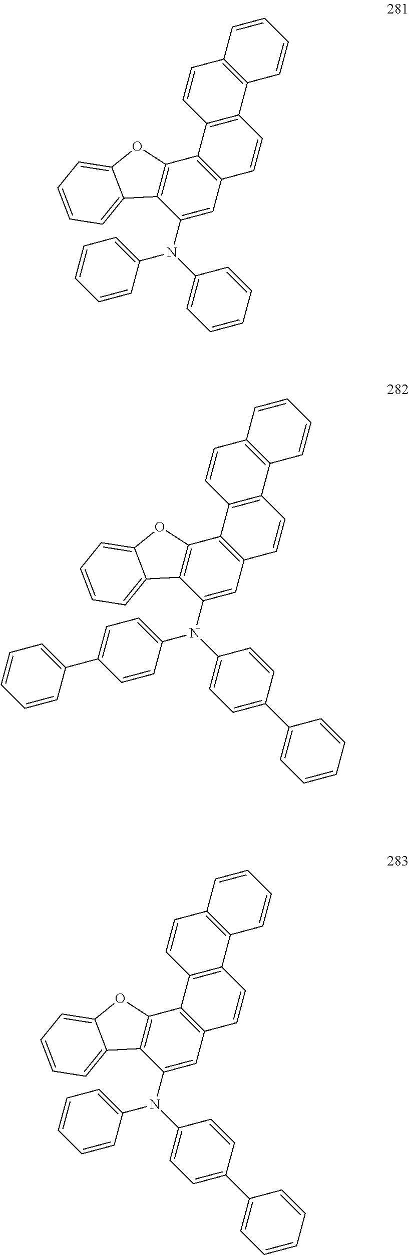 [SMILES: c1ccc(N(c2ccccc2)c2cc3ccc4c5ccccc5ccc4c3c3oc4ccccc4c23)cc1 ; c1ccc(-c2ccc(N(c3ccc(-c4ccccc4)cc3)c3cc4ccc5c6ccccc6ccc5c4c4oc5ccccc5c34)cc2)cc1 ; c1ccc(-c2ccc(N(c3ccccc3)c3cc4ccc5c6ccccc6ccc5c4c4oc5ccccc5c34)cc2)cc1]

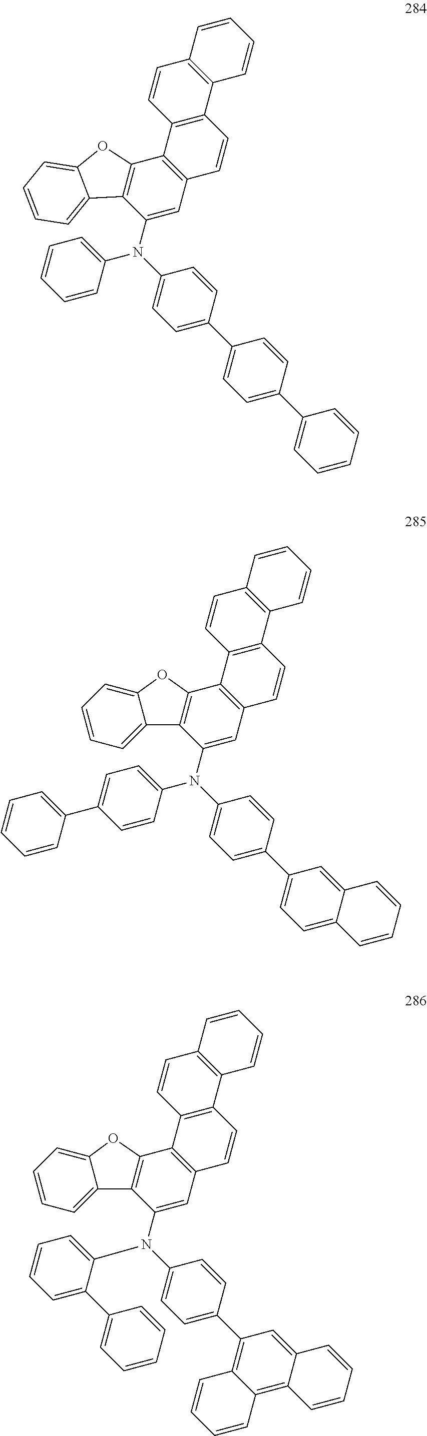 [SMILES: c1ccc(-c2ccc(-c3ccc(N(c4ccccc4)c4cc5ccc6c7ccccc7ccc6c5c5oc6ccccc6c45)cc3)cc2)cc1 ; c1ccc(-c2ccc(N(c3ccc(-c4ccc5ccccc5c4)cc3)c3cc4ccc5c6ccccc6ccc5c4c4oc5ccccc5c34)cc2)cc1 ; c1ccc(-c2ccccc2N(c2ccc(-c3cc4ccccc4c4ccccc34)cc2)c2cc3ccc4c5ccccc5ccc4c3c3oc4ccccc4c23)cc1]

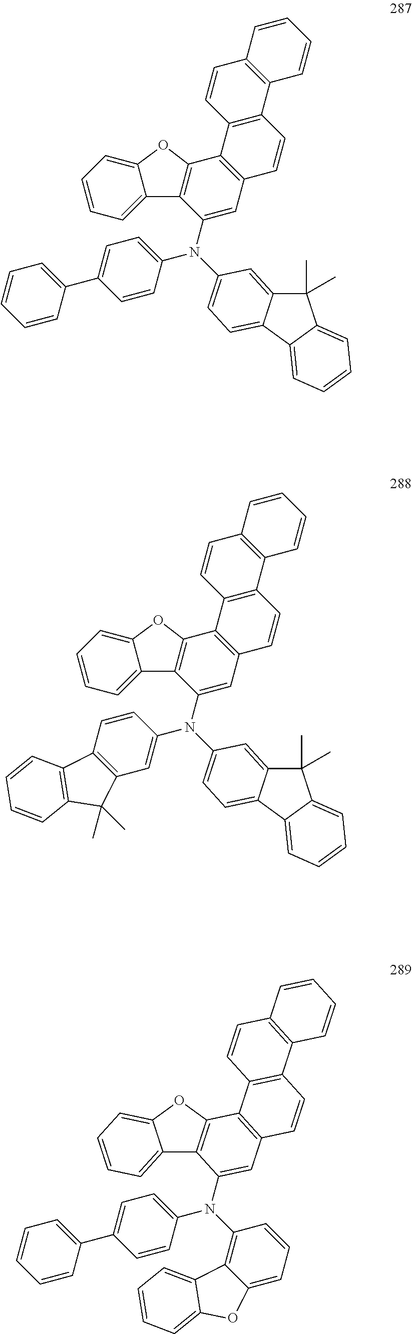 [SMILES: CC1(C)c2ccccc2-c2ccc(N(c3ccc(-c4ccccc4)cc3)c3cc4ccc5c6ccccc6ccc5c4c4oc5ccccc5c34)cc21 ; CC1(C)c2ccccc2-c2ccc(N(c3ccc4c(c3)C(C)(C)c3ccccc3-4)c3cc4ccc5c6ccccc6ccc5c4c4oc5ccccc5c34)cc21 ; c1ccc(-c2ccc(N(c3cccc4oc5ccccc5c34)c3cc4ccc5c6ccccc6ccc5c4c4oc5ccccc5c34)cc2)cc1]

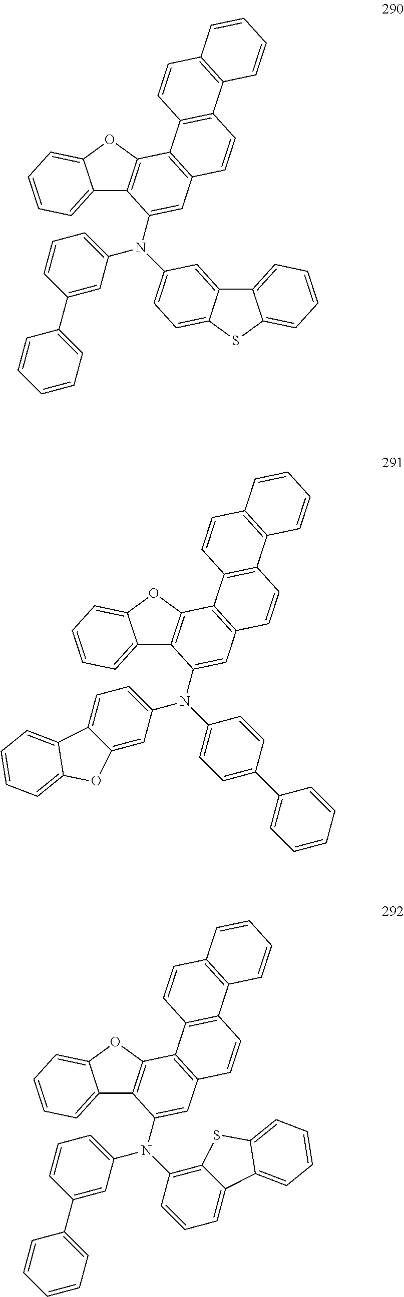 [SMILES: c1ccc(-c2cccc(N(c3ccc4sc5ccccc5c4c3)c3cc4ccc5c6ccccc6ccc5c4c4oc5ccccc5c34)c2)cc1 ; c1ccc(-c2ccc(N(c3ccc4c(c3)oc3ccccc34)c3cc4ccc5c6ccccc6ccc5c4c4oc5ccccc5c34)cc2)cc1 ; c1ccc(-c2cccc(N(c3cccc4c3sc3ccccc34)c3cc4ccc5c6ccccc6ccc5c4c4oc5ccccc5c34)c2)cc1]

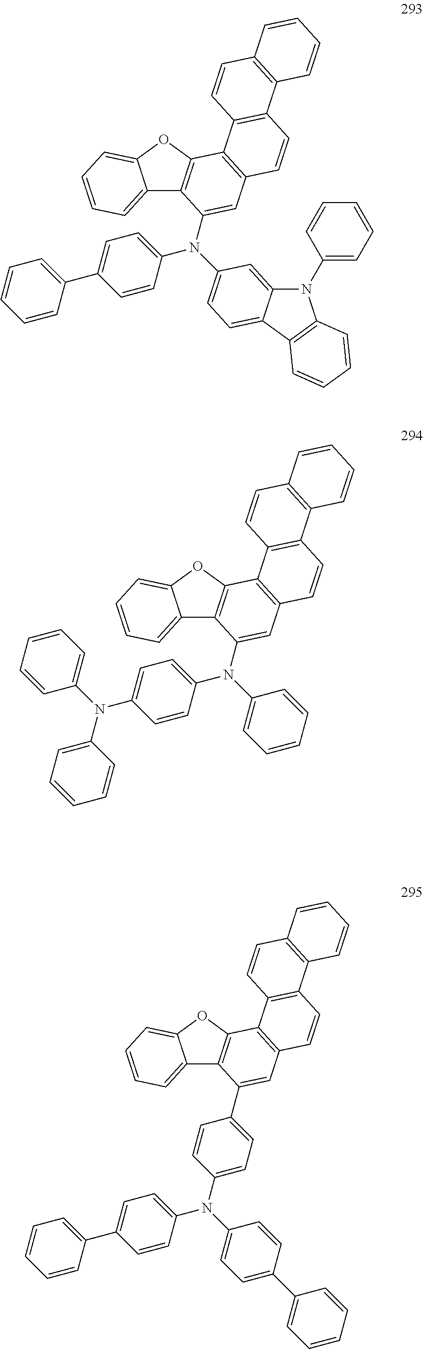 [SMILES: c1ccc(-c2ccc(N(c3ccc4c5ccccc5n(-c5ccccc5)c4c3)c3cc4ccc5c6ccccc6ccc5c4c4oc5ccccc5c34)cc2)cc1 ; c1ccc(N(c2ccccc2)c2ccc(N(c3ccccc3)c3cc4ccc5c6ccccc6ccc5c4c4oc5ccccc5c34)cc2)cc1 ; c1ccc(-c2ccc(N(c3ccc(-c4ccccc4)cc3)c3ccc(-c4cc5ccc6c7ccccc7ccc6c5c5oc6ccccc6c45)cc3)cc2)cc1]

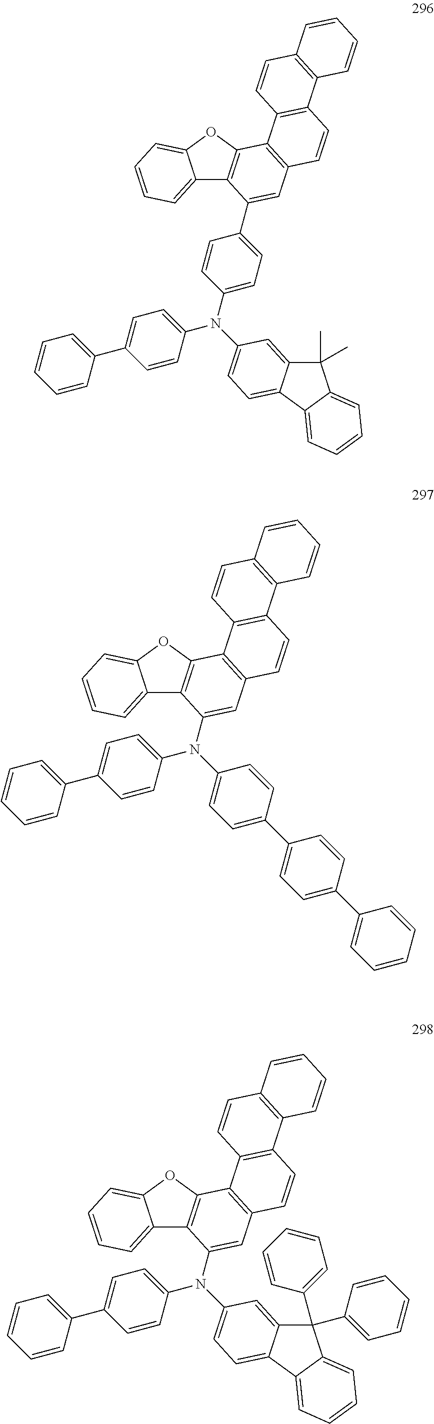 [SMILES: CC1(C)c2ccccc2-c2ccc(N(c3ccc(-c4ccccc4)cc3)c3ccc(-c4cc5ccc6c7ccccc7ccc6c5c5oc6ccccc6c45)cc3)cc21 ; c1ccc(-c2ccc(-c3ccc(N(c4ccc(-c5ccccc5)cc4)c4cc5ccc6c7ccccc7ccc6c5c5oc6ccccc6c45)cc3)cc2)cc1 ; c1ccc(-c2ccc(N(c3ccc4c(c3)C(c3ccccc3)(c3ccccc3)c3ccccc3-4)c3cc4ccc5c6ccccc6ccc5c4c4oc5ccccc5c34)cc2)cc1]

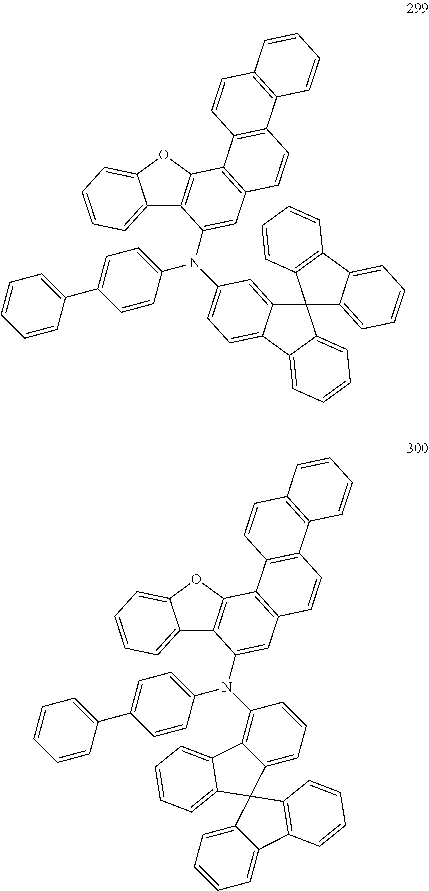 [SMILES: c1ccc(-c2ccc(N(c3ccc4c(c3)C3(c5ccccc5-c5ccccc53)c3ccccc3-4)c3cc4ccc5c6ccccc6ccc5c4c4oc5ccccc5c34)cc2)cc1 ; c1ccc(-c2ccc(N(c3cccc4c3-c3ccccc3C43c4ccccc4-c4ccccc43)c3cc4ccc5c6ccccc6ccc5c4c4oc5ccccc5c34)cc2)cc1]

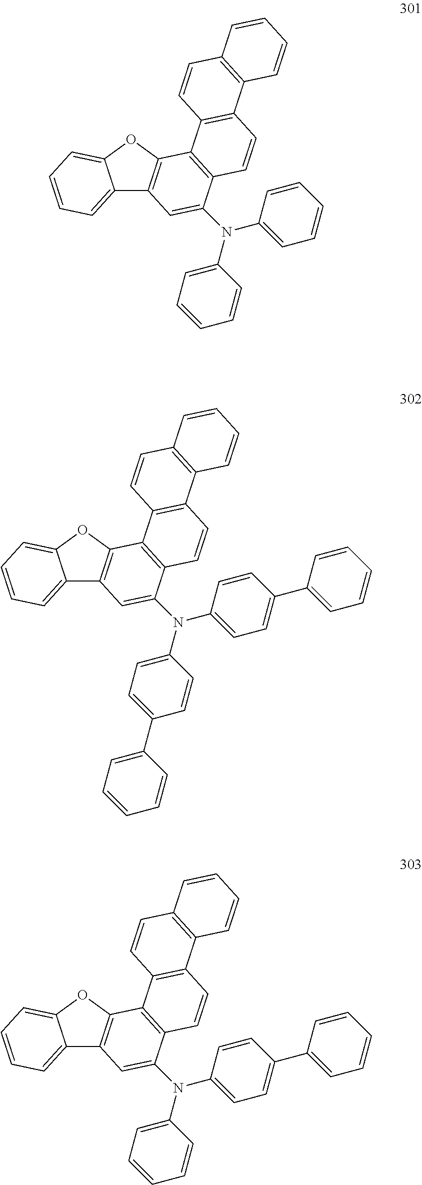 [SMILES: c1ccc(N(c2ccccc2)c2cc3c4ccccc4oc3c3c2ccc2c4ccccc4ccc23)cc1 ; c1ccc(-c2ccc(N(c3ccc(-c4ccccc4)cc3)c3cc4c5ccccc5oc4c4c3ccc3c5ccccc5ccc34)cc2)cc1 ; c1ccc(-c2ccc(N(c3ccccc3)c3cc4c5ccccc5oc4c4c3ccc3c5ccccc5ccc34)cc2)cc1]

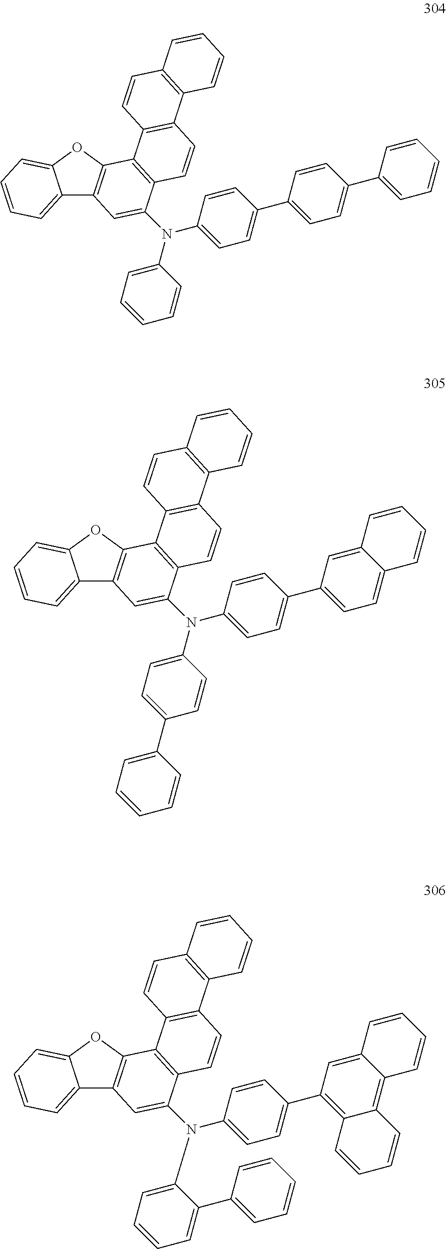 [SMILES: c1ccc(-c2ccc(-c3ccc(N(c4ccccc4)c4cc5c6ccccc6oc5c5c4ccc4c6ccccc6ccc45)cc3)cc2)cc1 ; c1ccc(-c2ccc(N(c3ccc(-c4ccc5ccccc5c4)cc3)c3cc4c5ccccc5oc4c4c3ccc3c5ccccc5ccc34)cc2)cc1 ; c1ccc(-c2ccccc2N(c2ccc(-c3cc4ccccc4c4ccccc34)cc2)c2cc3c4ccccc4oc3c3c2ccc2c4ccccc4ccc23)cc1]

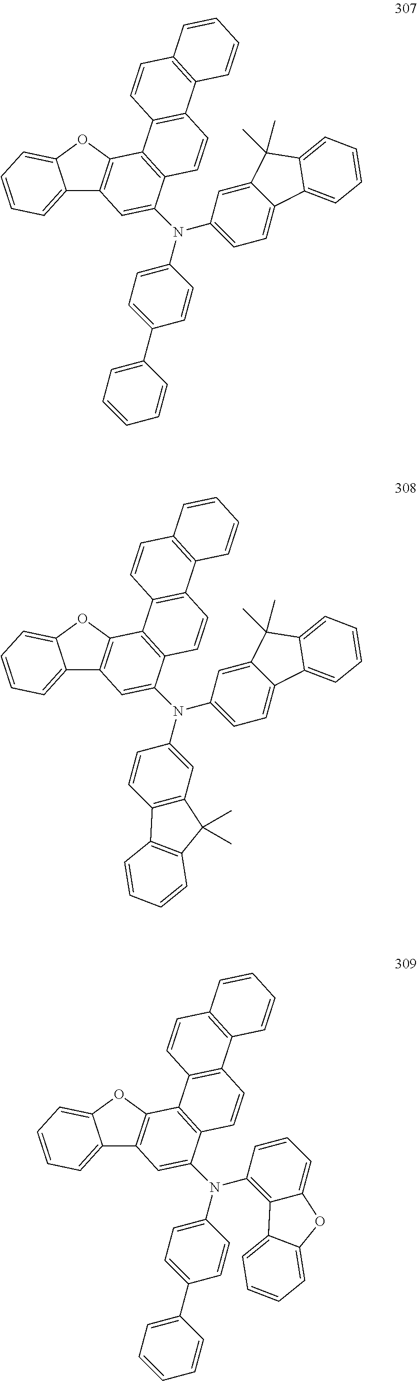 [SMILES: CC1(C)c2ccccc2-c2ccc(N(c3ccc(-c4ccccc4)cc3)c3cc4c5ccccc5oc4c4c3ccc3c5ccccc5ccc34)cc21 ; CC1(C)c2ccccc2-c2ccc(N(c3ccc4c(c3)C(C)(C)c3ccccc3-4)c3cc4c5ccccc5oc4c4c3ccc3c5ccccc5ccc34)cc21 ; c1ccc(-c2ccc(N(c3cc4c5ccccc5oc4c4c3ccc3c5ccccc5ccc34)c3cccc4oc5ccccc5c34)cc2)cc1]

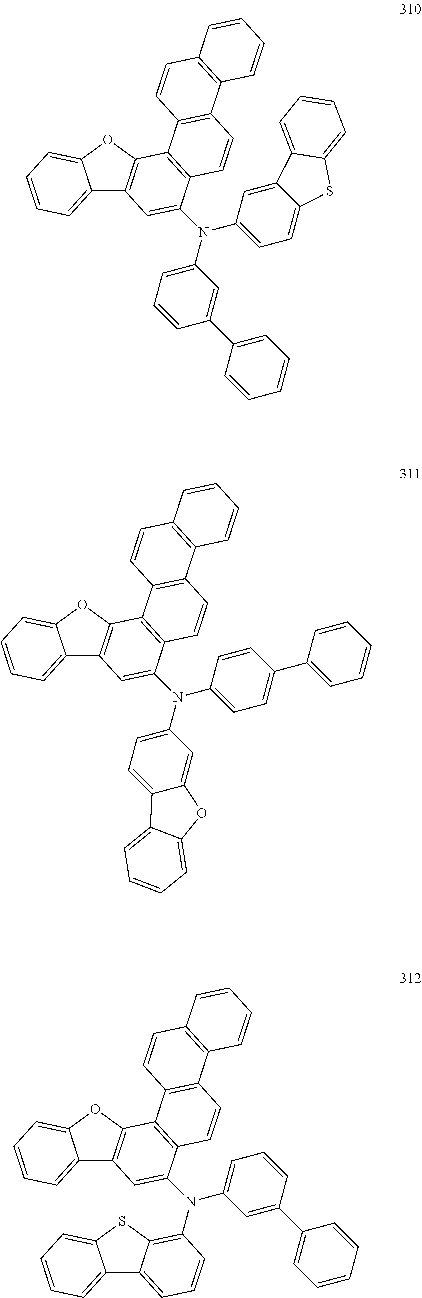 [SMILES: c1ccc(-c2cccc(N(c3ccc4sc5ccccc5c4c3)c3cc4c5ccccc5oc4c4c3ccc3c5ccccc5ccc34)c2)cc1 ; c1ccc(-c2ccc(N(c3ccc4c(c3)oc3ccccc34)c3cc4c5ccccc5oc4c4c3ccc3c5ccccc5ccc34)cc2)cc1 ; c1ccc(-c2cccc(N(c3cc4c5ccccc5oc4c4c3ccc3c5ccccc5ccc34)c3cccc4c3sc3ccccc34)c2)cc1]

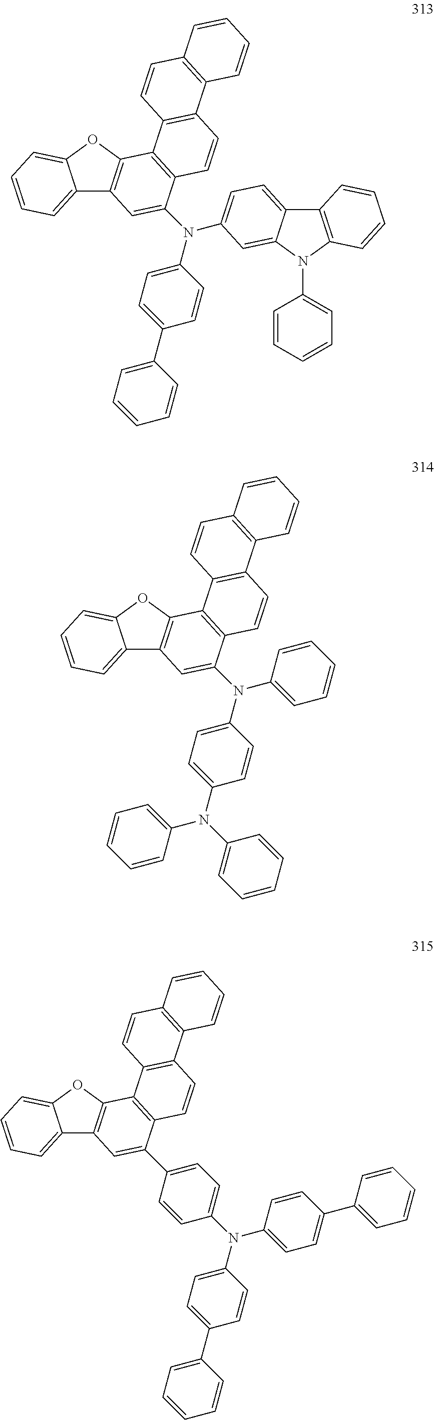 [SMILES: c1ccc(-c2ccc(N(c3ccc4c5ccccc5n(-c5ccccc5)c4c3)c3cc4c5ccccc5oc4c4c3ccc3c5ccccc5ccc34)cc2)cc1 ; c1ccc(N(c2ccccc2)c2ccc(N(c3ccccc3)c3cc4c5ccccc5oc4c4c3ccc3c5ccccc5ccc34)cc2)cc1 ; c1ccc(-c2ccc(N(c3ccc(-c4ccccc4)cc3)c3ccc(-c4cc5c6ccccc6oc5c5c4ccc4c6ccccc6ccc45)cc3)cc2)cc1]

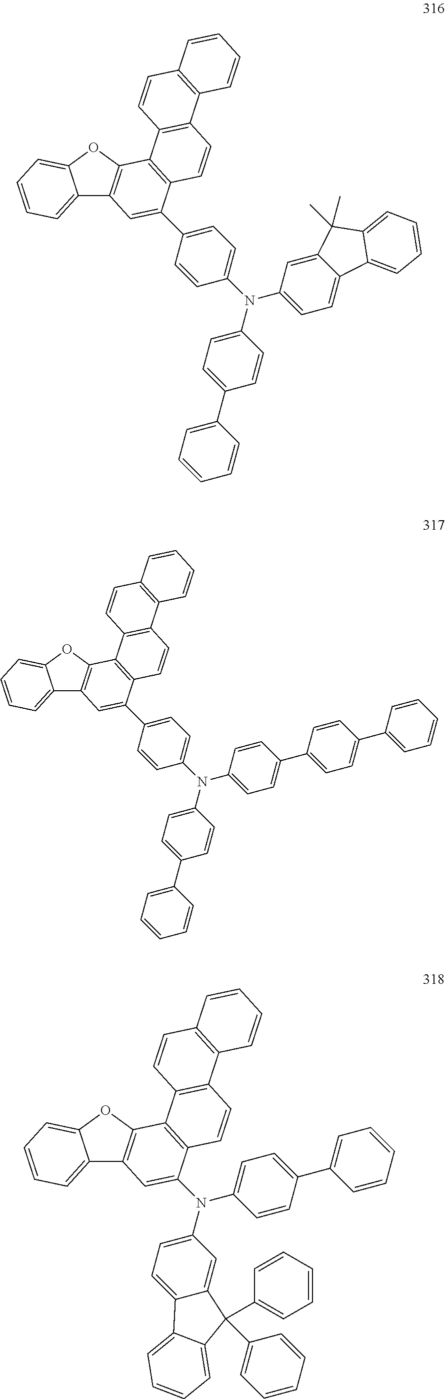 [SMILES: CC1(C)c2ccccc2-c2ccc(N(c3ccc(-c4ccccc4)cc3)c3ccc(-c4cc5c6ccccc6oc5c5c4ccc4c6ccccc6ccc45)cc3)cc21 ; c1ccc(-c2ccc(-c3ccc(N(c4ccc(-c5ccccc5)cc4)c4ccc(-c5cc6c7ccccc7oc6c6c5ccc5c7ccccc7ccc56)cc4)cc3)cc2)cc1 ; c1ccc(-c2ccc(N(c3ccc4c(c3)C(c3ccccc3)(c3ccccc3)c3ccccc3-4)c3cc4c5ccccc5oc4c4c3ccc3c5ccccc5ccc34)cc2)cc1]

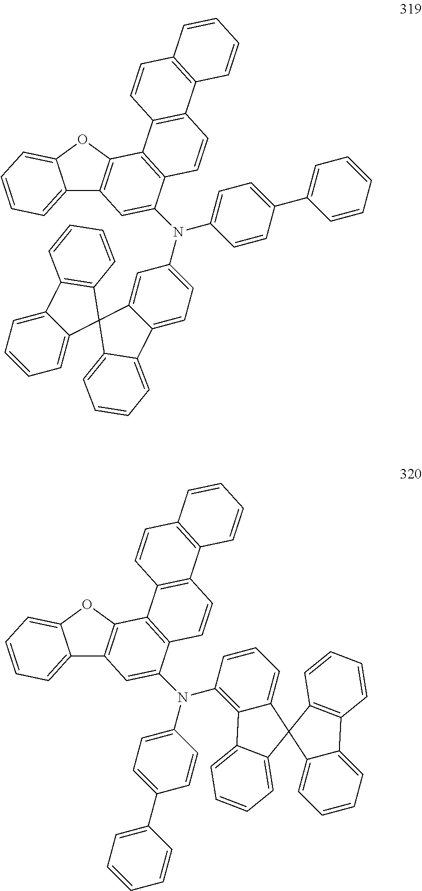 [SMILES: c1ccc(-c2ccc(N(c3ccc4c(c3)C3(c5ccccc5-c5ccccc53)c3ccccc3-4)c3cc4c5ccccc5oc4c4c3ccc3c5ccccc5ccc34)cc2)cc1 ; c1ccc(-c2ccc(N(c3cccc4c3-c3ccccc3C43c4ccccc4-c4ccccc43)c3cc4c5ccccc5oc4c4c3ccc3c5ccccc5ccc34)cc2)cc1]

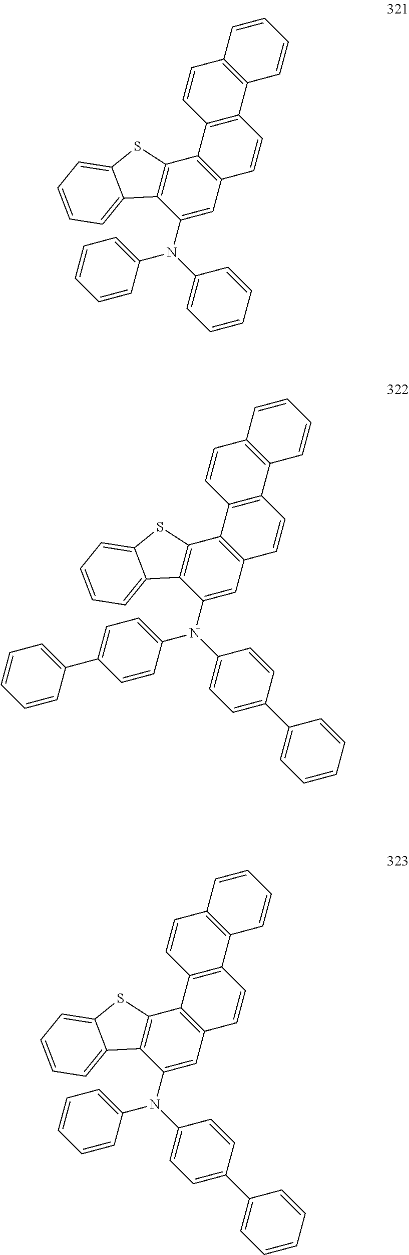 [SMILES: c1ccc(N(c2ccccc2)c2cc3ccc4c5ccccc5ccc4c3c3sc4ccccc4c23)cc1 ; c1ccc(-c2ccc(N(c3ccc(-c4ccccc4)cc3)c3cc4ccc5c6ccccc6ccc5c4c4sc5ccccc5c34)cc2)cc1 ; c1ccc(-c2ccc(N(c3ccccc3)c3cc4ccc5c6ccccc6ccc5c4c4sc5ccccc5c34)cc2)cc1]

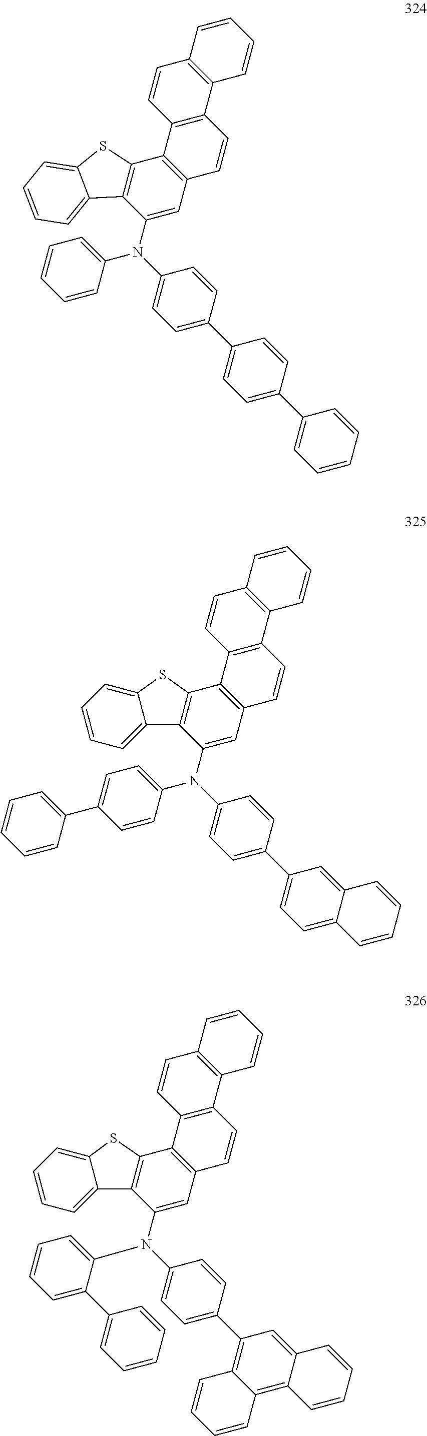 [SMILES: c1ccc(-c2ccc(-c3ccc(N(c4ccccc4)c4cc5ccc6c7ccccc7ccc6c5c5sc6ccccc6c45)cc3)cc2)cc1 ; c1ccc(-c2ccc(N(c3ccc(-c4ccc5ccccc5c4)cc3)c3cc4ccc5c6ccccc6ccc5c4c4sc5ccccc5c34)cc2)cc1 ; c1ccc(-c2ccccc2N(c2ccc(-c3cc4ccccc4c4ccccc34)cc2)c2cc3ccc4c5ccccc5ccc4c3c3sc4ccccc4c23)cc1]

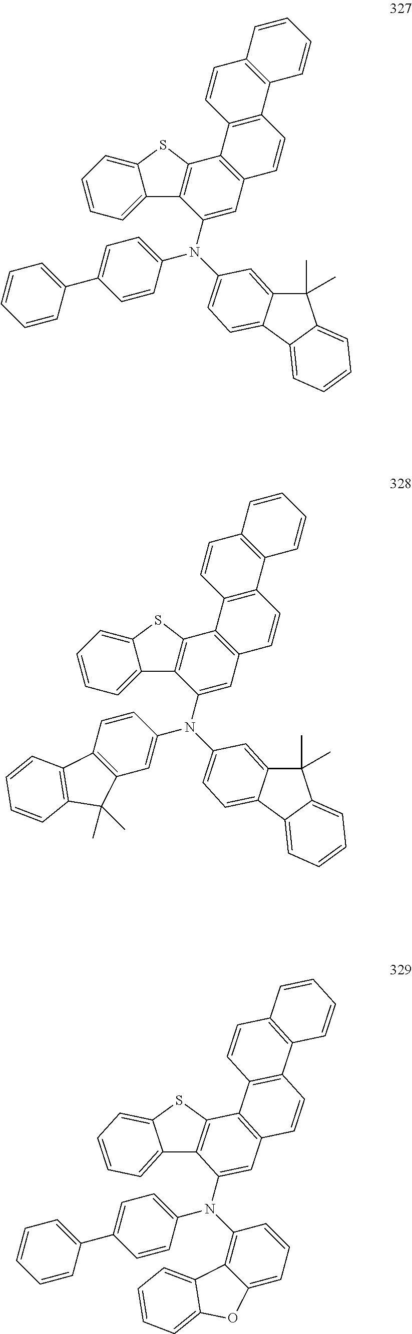 [SMILES: CC1(C)c2ccccc2-c2ccc(N(c3ccc(-c4ccccc4)cc3)c3cc4ccc5c6ccccc6ccc5c4c4sc5ccccc5c34)cc21 ; CC1(C)c2ccccc2-c2ccc(N(c3ccc4c(c3)C(C)(C)c3ccccc3-4)c3cc4ccc5c6ccccc6ccc5c4c4sc5ccccc5c34)cc21 ; c1ccc(-c2ccc(N(c3cccc4oc5ccccc5c34)c3cc4ccc5c6ccccc6ccc5c4c4sc5ccccc5c34)cc2)cc1]

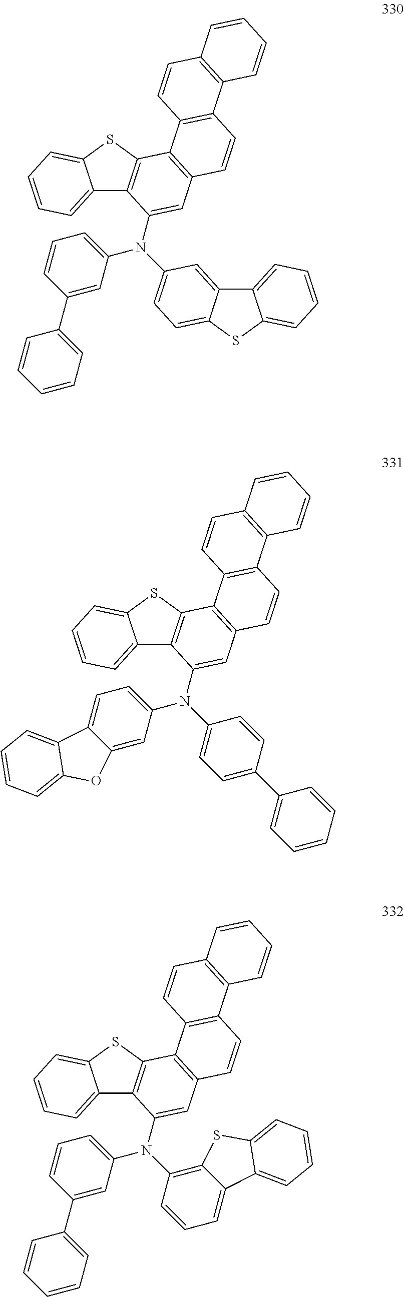 [SMILES: c1ccc(-c2cccc(N(c3ccc4sc5ccccc5c4c3)c3cc4ccc5c6ccccc6ccc5c4c4sc5ccccc5c34)c2)cc1 ; c1ccc(-c2ccc(N(c3ccc4c(c3)oc3ccccc34)c3cc4ccc5c6ccccc6ccc5c4c4sc5ccccc5c34)cc2)cc1 ; c1ccc(-c2cccc(N(c3cccc4c3sc3ccccc34)c3cc4ccc5c6ccccc6ccc5c4c4sc5ccccc5c34)c2)cc1]

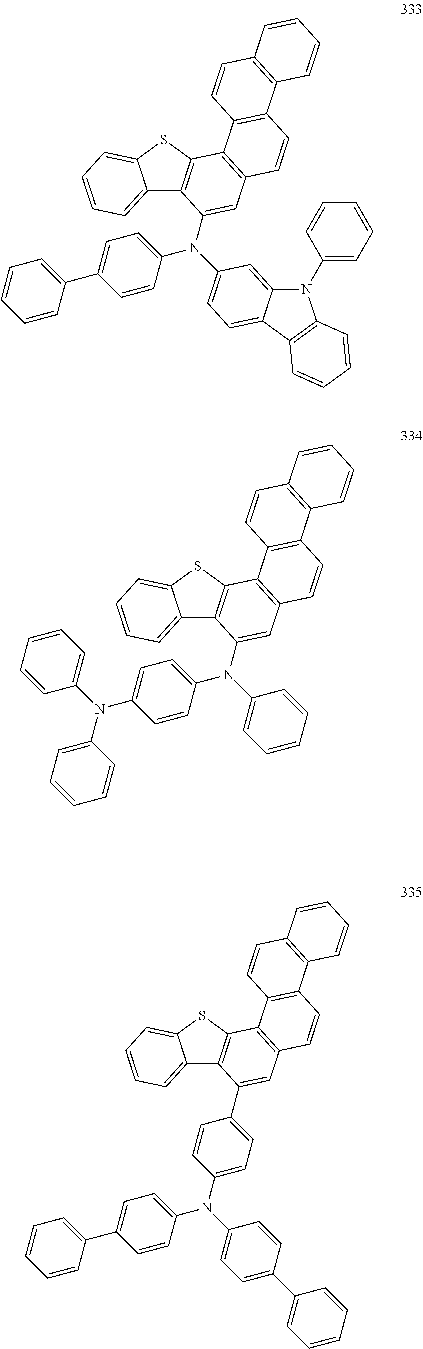 [SMILES: c1ccc(-c2ccc(N(c3ccc4c5ccccc5n(-c5ccccc5)c4c3)c3cc4ccc5c6ccccc6ccc5c4c4sc5ccccc5c34)cc2)cc1 ; c1ccc(N(c2ccccc2)c2ccc(N(c3ccccc3)c3cc4ccc5c6ccccc6ccc5c4c4sc5ccccc5c34)cc2)cc1 ; c1ccc(-c2ccc(N(c3ccc(-c4ccccc4)cc3)c3ccc(-c4cc5ccc6c7ccccc7ccc6c5c5sc6ccccc6c45)cc3)cc2)cc1]

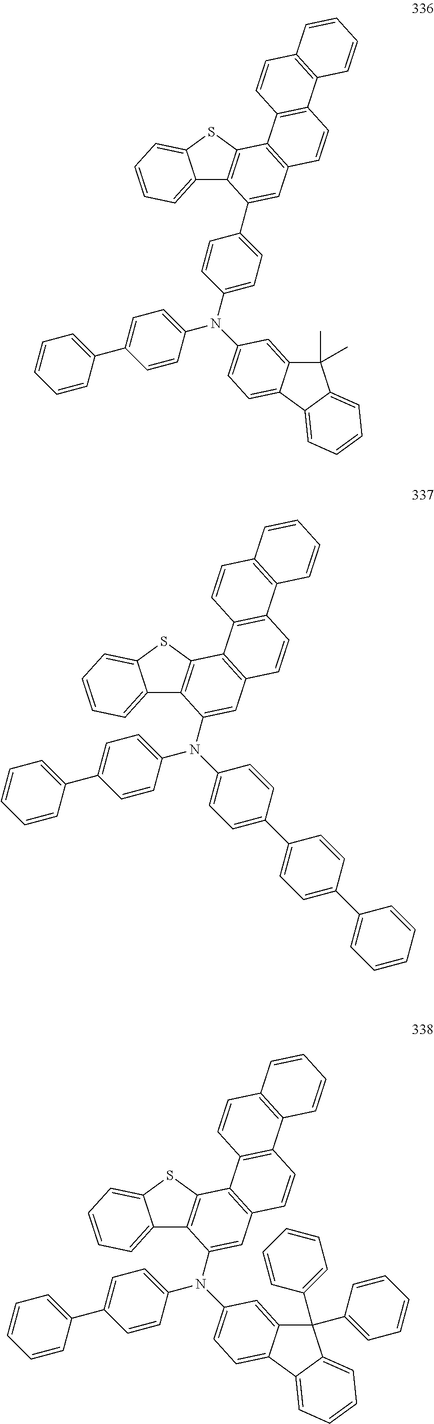 [SMILES: CC1(C)c2ccccc2-c2ccc(N(c3ccc(-c4ccccc4)cc3)c3ccc(-c4cc5ccc6c7ccccc7ccc6c5c5sc6ccccc6c45)cc3)cc21 ; c1ccc(-c2ccc(-c3ccc(N(c4ccc(-c5ccccc5)cc4)c4cc5ccc6c7ccccc7ccc6c5c5sc6ccccc6c45)cc3)cc2)cc1 ; c1ccc(-c2ccc(N(c3ccc4c(c3)C(c3ccccc3)(c3ccccc3)c3ccccc3-4)c3cc4ccc5c6ccccc6ccc5c4c4sc5ccccc5c34)cc2)cc1]

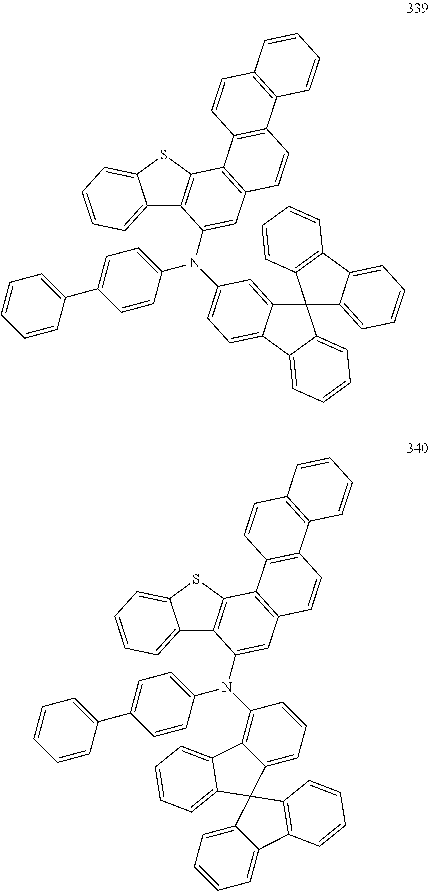 [SMILES: c1ccc(-c2ccc(N(c3ccc4c(c3)C3(c5ccccc5-c5ccccc53)c3ccccc3-4)c3cc4ccc5c6ccccc6ccc5c4c4sc5ccccc5c34)cc2)cc1 ; c1ccc(-c2ccc(N(c3cccc4c3-c3ccccc3C43c4ccccc4-c4ccccc43)c3cc4ccc5c6ccccc6ccc5c4c4sc5ccccc5c34)cc2)cc1]

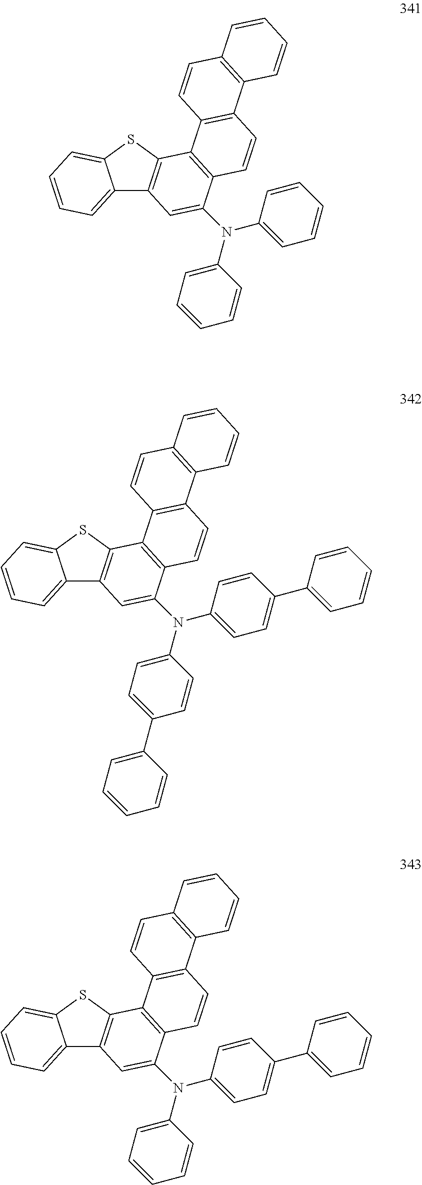 [SMILES: c1ccc(N(c2ccccc2)c2cc3c4ccccc4sc3c3c2ccc2c4ccccc4ccc23)cc1 ; c1ccc(-c2ccc(N(c3ccc(-c4ccccc4)cc3)c3cc4c5ccccc5sc4c4c3ccc3c5ccccc5ccc34)cc2)cc1 ; c1ccc(-c2ccc(N(c3ccccc3)c3cc4c5ccccc5sc4c4c3ccc3c5ccccc5ccc34)cc2)cc1]

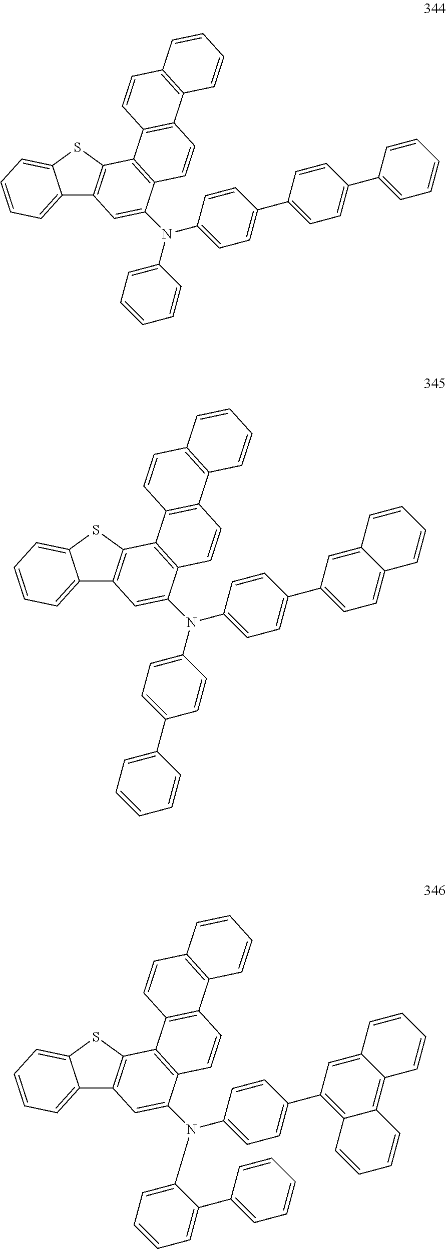 [SMILES: c1ccc(-c2ccc(-c3ccc(N(c4ccccc4)c4cc5c6ccccc6sc5c5c4ccc4c6ccccc6ccc45)cc3)cc2)cc1 ; c1ccc(-c2ccc(N(c3ccc(-c4ccc5ccccc5c4)cc3)c3cc4c5ccccc5sc4c4c3ccc3c5ccccc5ccc34)cc2)cc1 ; c1ccc(-c2ccccc2N(c2ccc(-c3cc4ccccc4c4ccccc34)cc2)c2cc3c4ccccc4sc3c3c2ccc2c4ccccc4ccc23)cc1]

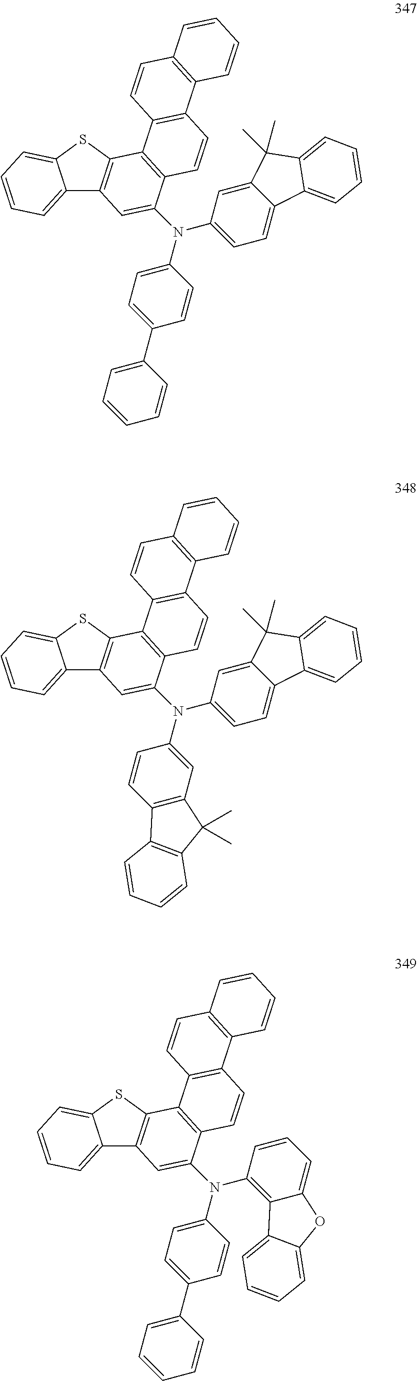 [SMILES: CC1(C)c2ccccc2-c2ccc(N(c3ccc(-c4ccccc4)cc3)c3cc4c5ccccc5sc4c4c3ccc3c5ccccc5ccc34)cc21 ; CC1(C)c2ccccc2-c2ccc(N(c3ccc4c(c3)C(C)(C)c3ccccc3-4)c3cc4c5ccccc5sc4c4c3ccc3c5ccccc5ccc34)cc21 ; c1ccc(-c2ccc(N(c3cc4c5ccccc5sc4c4c3ccc3c5ccccc5ccc34)c3cccc4oc5ccccc5c34)cc2)cc1]

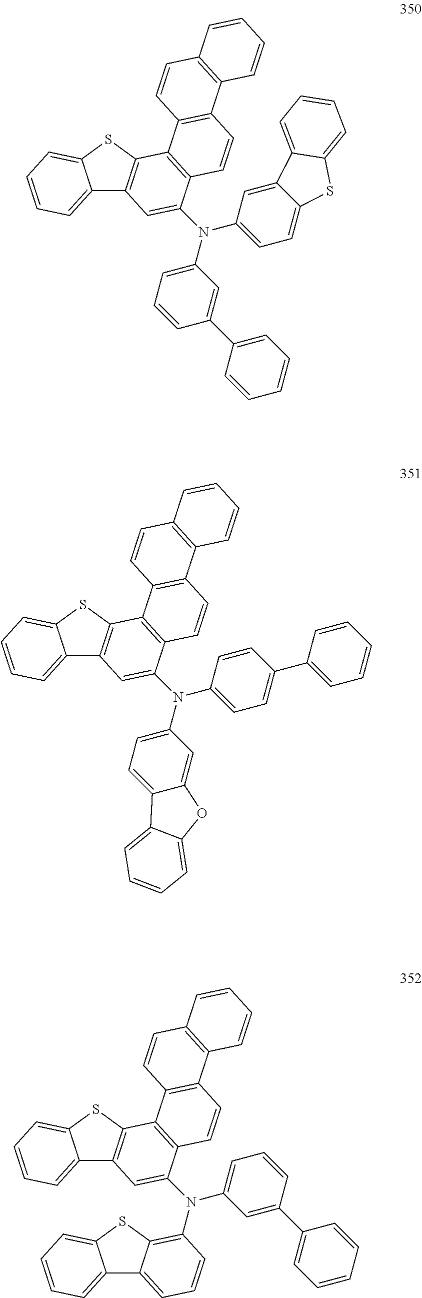 [SMILES: c1ccc(-c2cccc(N(c3ccc4sc5ccccc5c4c3)c3cc4c5ccccc5sc4c4c3ccc3c5ccccc5ccc34)c2)cc1 ; c1ccc(-c2ccc(N(c3ccc4c(c3)oc3ccccc34)c3cc4c5ccccc5sc4c4c3ccc3c5ccccc5ccc34)cc2)cc1 ; c1ccc(-c2cccc(N(c3cc4c5ccccc5sc4c4c3ccc3c5ccccc5ccc34)c3cccc4c3sc3ccccc34)c2)cc1]

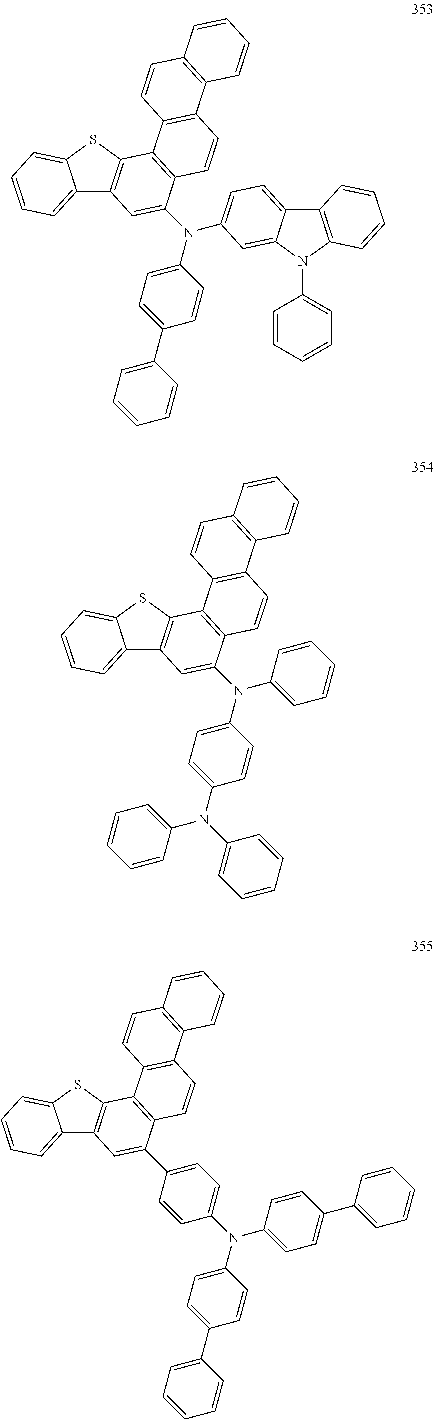 [SMILES: c1ccc(-c2ccc(N(c3ccc4c5ccccc5n(-c5ccccc5)c4c3)c3cc4c5ccccc5sc4c4c3ccc3c5ccccc5ccc34)cc2)cc1 ; c1ccc(N(c2ccccc2)c2ccc(N(c3ccccc3)c3cc4c5ccccc5sc4c4c3ccc3c5ccccc5ccc34)cc2)cc1 ; c1ccc(-c2ccc(N(c3ccc(-c4ccccc4)cc3)c3ccc(-c4cc5c6ccccc6sc5c5c4ccc4c6ccccc6ccc45)cc3)cc2)cc1]

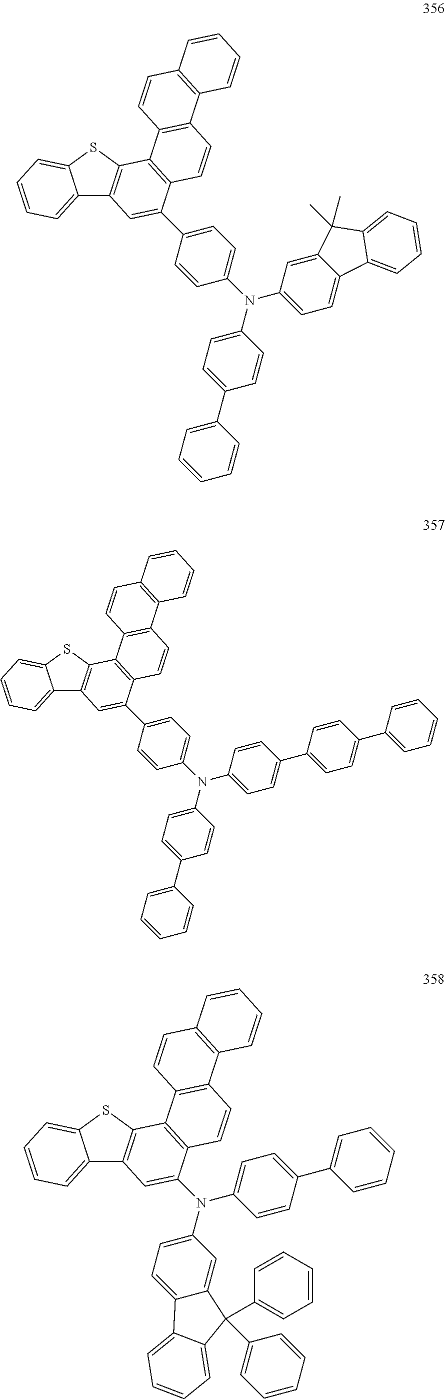 [SMILES: CC1(C)c2ccccc2-c2ccc(N(c3ccc(-c4ccccc4)cc3)c3ccc(-c4cc5c6ccccc6sc5c5c4ccc4c6ccccc6ccc45)cc3)cc21 ; c1ccc(-c2ccc(-c3ccc(N(c4ccc(-c5ccccc5)cc4)c4ccc(-c5cc6c7ccccc7sc6c6c5ccc5c7ccccc7ccc56)cc4)cc3)cc2)cc1 ; c1ccc(-c2ccc(N(c3ccc4c(c3)C(c3ccccc3)(c3ccccc3)c3ccccc3-4)c3cc4c5ccccc5sc4c4c3ccc3c5ccccc5ccc34)cc2)cc1]

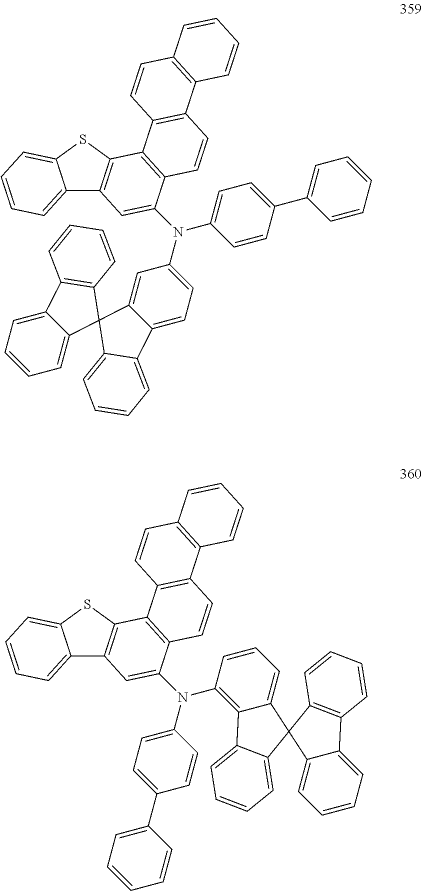 [SMILES: c1ccc(-c2ccc(N(c3ccc4c(c3)C3(c5ccccc5-c5ccccc53)c3ccccc3-4)c3cc4c5ccccc5sc4c4c3ccc3c5ccccc5ccc34)cc2)cc1 ; c1ccc(-c2ccc(N(c3cccc4c3-c3ccccc3C43c4ccccc4-c4ccccc43)c3cc4c5ccccc5sc4c4c3ccc3c5ccccc5ccc34)cc2)cc1]

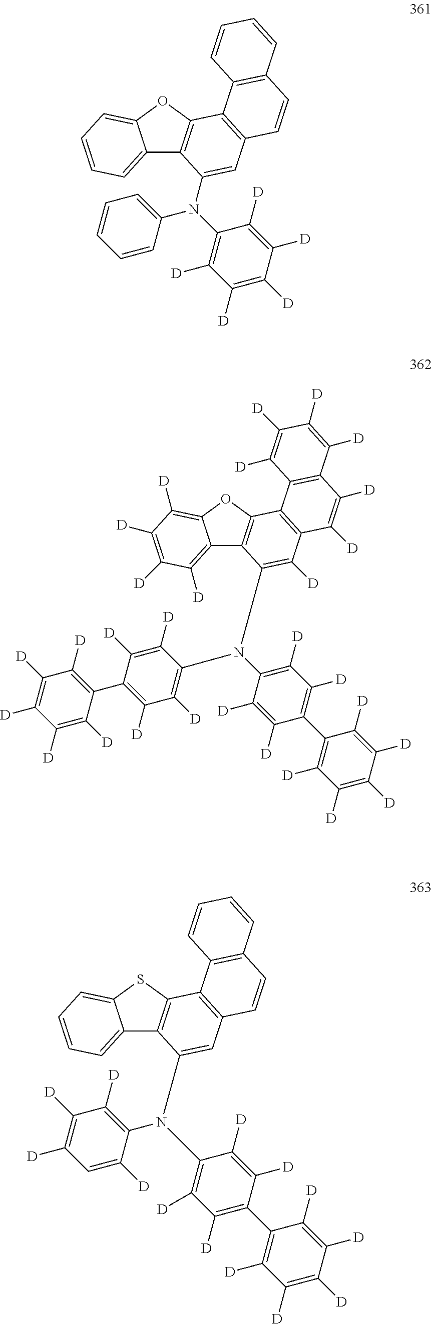 [SMILES: [2H]c1c([2H])c([2H])c(N(c2ccccc2)c2cc3ccc4ccccc4c3c3oc4ccccc4c23)c([2H])c1[2H] ; [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c4c([2H])c([2H])c5c([2H])c([2H])c([2H])c([2H])c5c4c4oc5c([2H])c([2H])c([2H])c([2H])c5c34)c([2H])c2[2H])c([2H])c1[2H] ; [2H]c1cc([2H])c(N(c2c([2H])c([2H])c(-c3c([2H])c([2H])c([2H])c([2H])c3[2H])c([2H])c2[2H])c2cc3ccc4ccccc4c3c3sc4ccccc4c23)c([2H])c1[2H]]

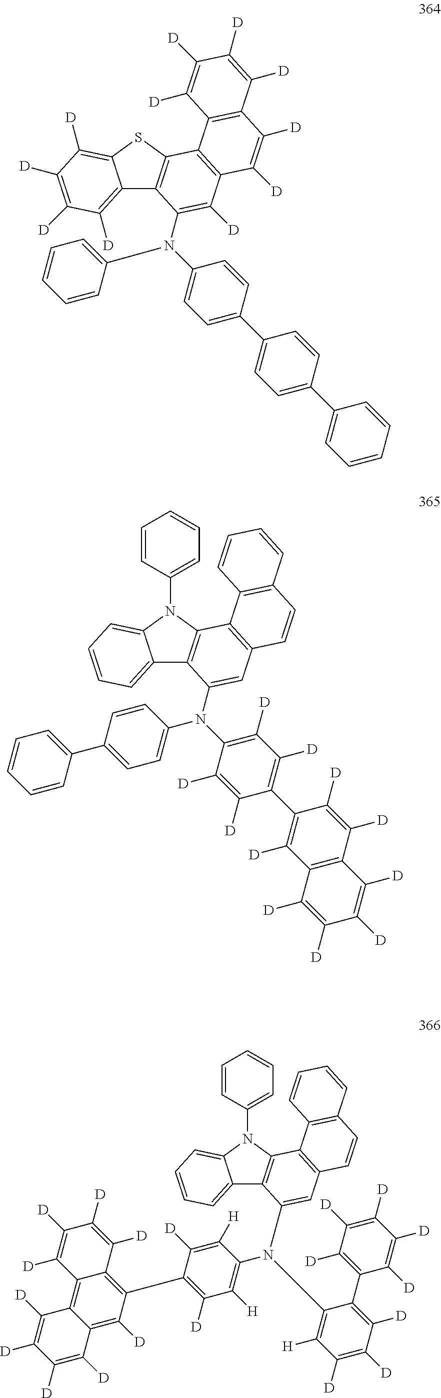 [SMILES: [2H]c1c([2H])c([2H])c2c(sc3c2c(N(c2ccccc2)c2ccc(-c4ccc(-c5ccccc5)cc4)cc2)c([2H])c2c([2H])c([2H])c4c([2H])c([2H])c([2H])c([2H])c4c23)c1[2H] ; [2H]c1c([2H])c(N(c2ccc(-c3ccccc3)cc2)c2cc3ccc4ccccc4c3c3c2c2ccccc2n3-c2ccccc2)c([2H])c([2H])c1-c1c([2H])c([2H])c2c([2H])c([2H])c([2H])c([2H])c2c1[2H] ; [2H]c1cc(N(c2cc([2H])c(-c3c([2H])c4c([2H])c([2H])c([2H])c([2H])c4c4c([2H])c([2H])c([2H])c([2H])c34)c([2H])c2)c2cc3ccc4ccccc4c3c3c2c2ccccc2n3-c2ccccc2)c(-c2c([2H])c([2H])c([2H])c([2H])c2[2H])c([2H])c1[2H]]

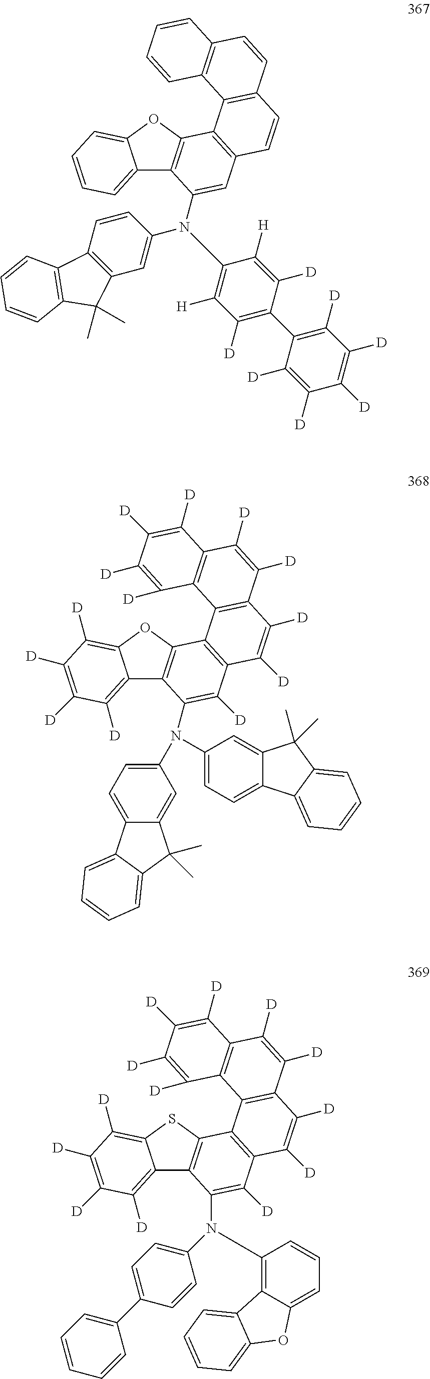 [SMILES: [2H]c1cc(N(c2ccc3c(c2)C(C)(C)c2ccccc2-3)c2cc3ccc4ccc5ccccc5c4c3c3oc4ccccc4c23)cc([2H])c1-c1c([2H])c([2H])c([2H])c([2H])c1[2H] ; [2H]c1c([2H])c([2H])c2c(oc3c2c(N(c2ccc4c(c2)C(C)(C)c2ccccc2-4)c2ccc4c(c2)C(C)(C)c2ccccc2-4)c([2H])c2c([2H])c([2H])c4c([2H])c([2H])c5c([2H])c([2H])c([2H])c([2H])c5c4c23)c1[2H] ; [2H]c1c([2H])c([2H])c2c(sc3c2c(N(c2ccc(-c4ccccc4)cc2)c2cccc4oc5ccccc5c24)c([2H])c2c([2H])c([2H])c4c([2H])c([2H])c5c([2H])c([2H])c([2H])c([2H])c5c4c23)c1[2H]]

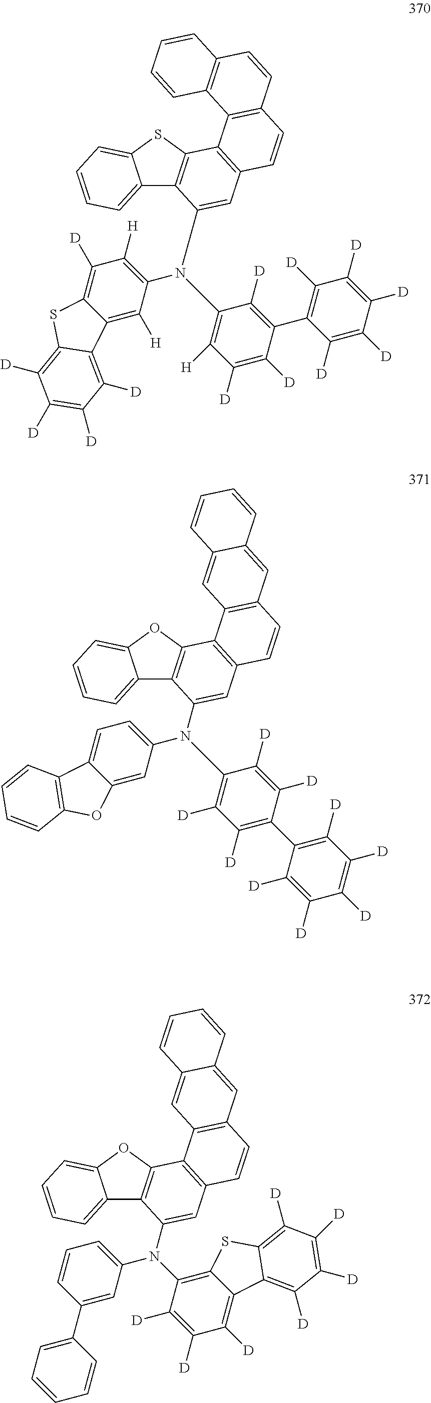 [SMILES: [2H]c1cc(N(c2cc([2H])c3sc4c([2H])c([2H])c([2H])c([2H])c4c3c2)c2cc3ccc4ccc5ccccc5c4c3c3sc4ccccc4c23)c([2H])c(-c2c([2H])c([2H])c([2H])c([2H])c2[2H])c1[2H] ; [2H]c1c([2H])c([2H])c(-c2c([2H])c([2H])c(N(c3ccc4c(c3)oc3ccccc34)c3cc4ccc5cc6ccccc6cc5c4c4oc5ccccc5c34)c([2H])c2[2H])c([2H])c1[2H] ; [2H]c1c([2H])c([2H])c2c(sc3c(N(c4cccc(-c5ccccc5)c4)c4cc5ccc6cc7ccccc7cc6c5c5oc6ccccc6c45)c([2H])c([2H])c([2H])c32)c1[2H]]

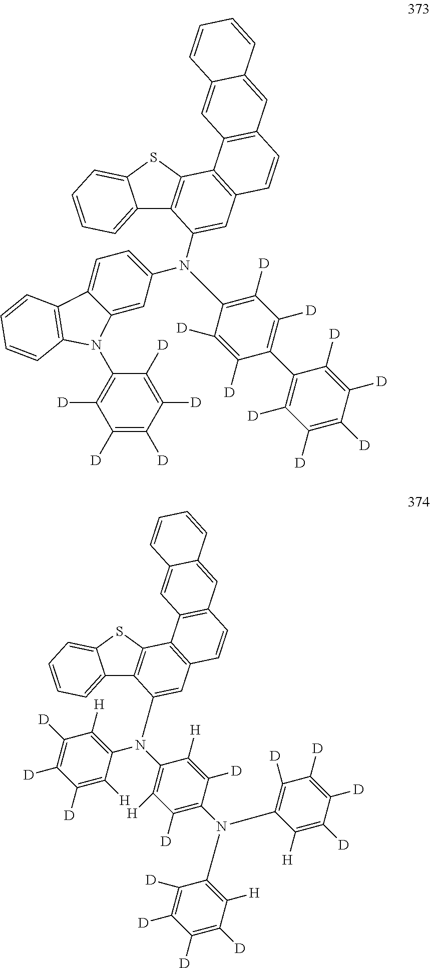 [SMILES: [2H]c1c([2H])c([2H])c(-c2c([2H])c([2H])c(N(c3ccc4c5ccccc5n(-c5c([2H])c([2H])c([2H])c([2H])c5[2H])c4c3)c3cc4ccc5cc6ccccc6cc5c4c4sc5ccccc5c34)c([2H])c2[2H])c([2H])c1[2H] ; [2H]c1cc(N(c2cc([2H])c(N(c3cc([2H])c([2H])c([2H])c3[2H])c3cc([2H])c([2H])c([2H])c3[2H])c([2H])c2)c2cc3ccc4cc5ccccc5cc4c3c3sc4ccccc4c23)cc([2H])c1[2H]]

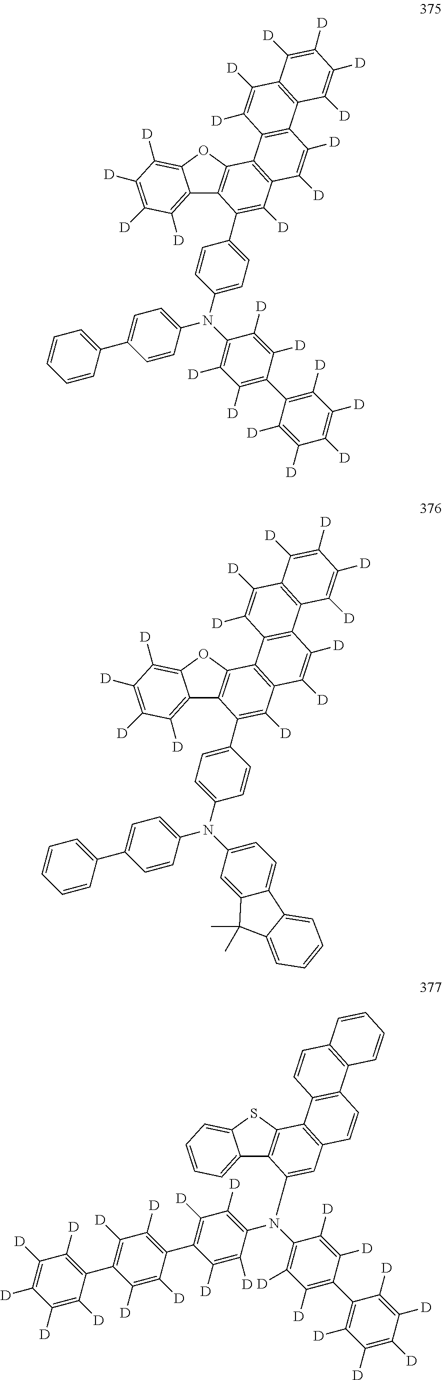

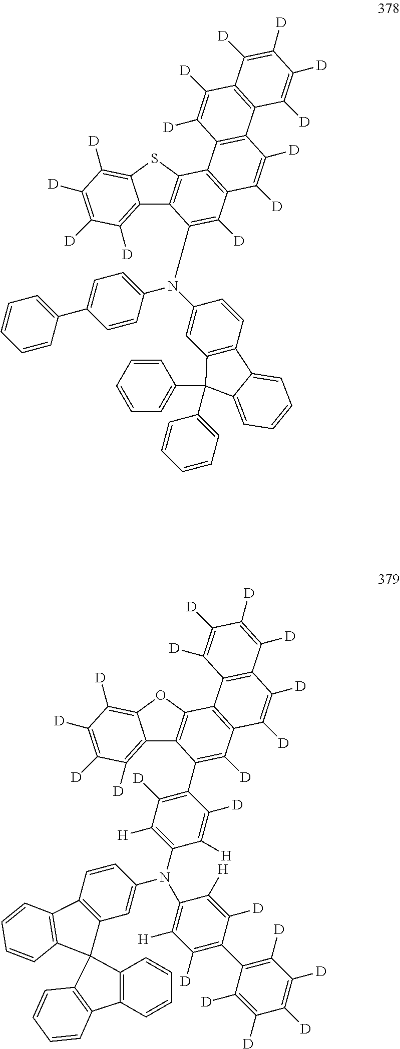 [SMILES: [2H]c1c([2H])c([2H])c2c(sc3c2c(N(c2ccc(-c4ccccc4)cc2)c2ccc4c(c2)C(c2ccccc2)(c2ccccc2)c2ccccc2-4)c([2H])c2c([2H])c([2H])c4c5c([2H])c([2H])c([2H])c([2H])c5c([2H])c([2H])c4c23)c1[2H] ; [2H]c1cc(N(c2cc([2H])c(-c3c([2H])c4c([2H])c([2H])c5c([2H])c([2H])c([2H])c([2H])c5c4c4oc5c([2H])c([2H])c([2H])c([2H])c5c34)c([2H])c2)c2ccc3c(c2)C2(c4ccccc4-c4ccccc42)c2ccccc2-3)cc([2H])c1-c1c([2H])c([2H])c([2H])c([2H])c1[2H]]

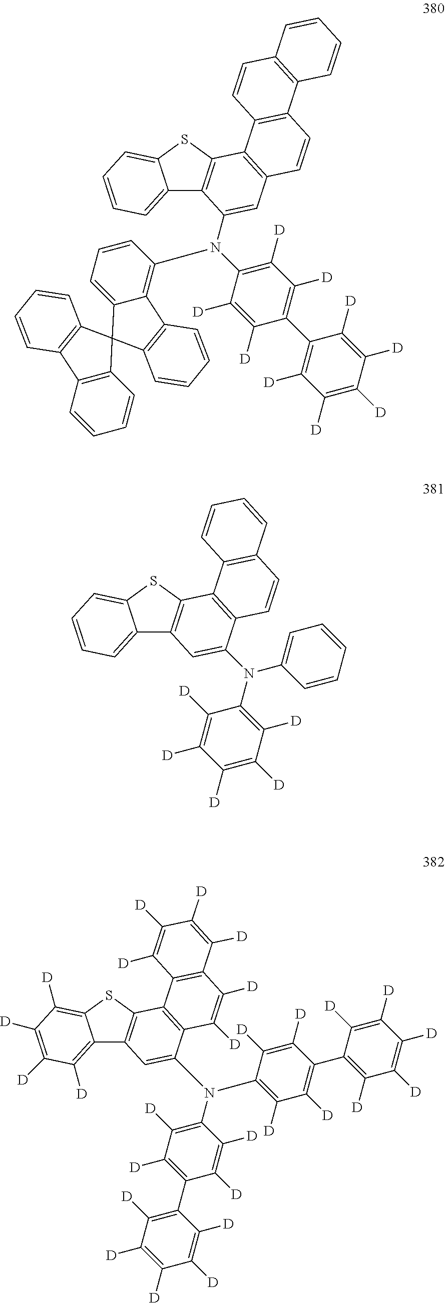 [SMILES: [2H]c1c([2H])c([2H])c(-c2c([2H])c([2H])c(N(c3cccc4c3-c3ccccc3C43c4ccccc4-c4ccccc43)c3cc4ccc5c6ccccc6ccc5c4c4sc5ccccc5c34)c([2H])c2[2H])c([2H])c1[2H] ; [2H]c1c([2H])c([2H])c(N(c2ccccc2)c2cc3c4ccccc4sc3c3c2ccc2ccccc23)c([2H])c1[2H] ; [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3cc4c(sc5c([2H])c([2H])c([2H])c([2H])c54)c4c3c([2H])c([2H])c3c([2H])c([2H])c([2H])c([2H])c34)c([2H])c2[2H])c([2H])c1[2H]]

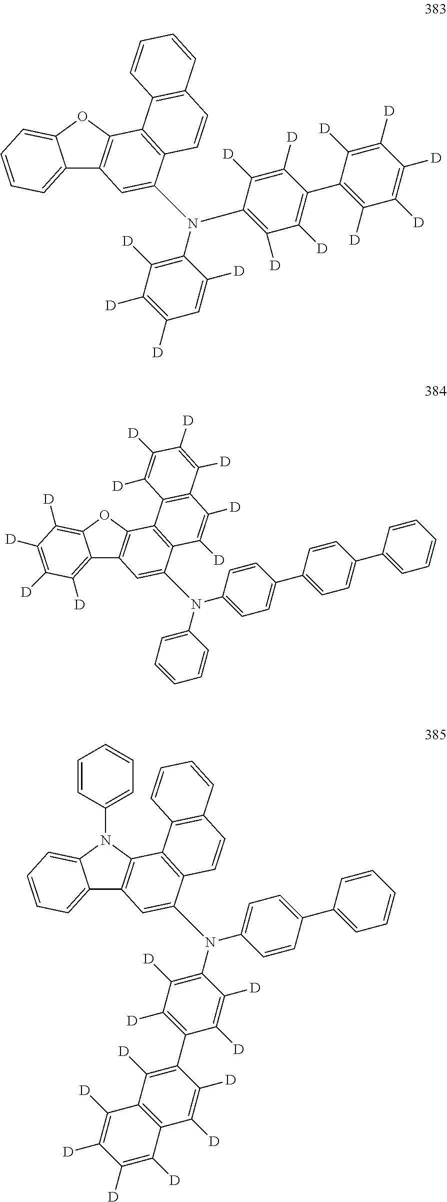 [SMILES: [2H]c1cc([2H])c(N(c2c([2H])c([2H])c(-c3c([2H])c([2H])c([2H])c([2H])c3[2H])c([2H])c2[2H])c2cc3c4ccccc4oc3c3c2ccc2ccccc23)c([2H])c1[2H] ; [2H]c1c([2H])c([2H])c2c(oc3c2cc(N(c2ccccc2)c2ccc(-c4ccc(-c5ccccc5)cc4)cc2)c2c([2H])c([2H])c4c([2H])c([2H])c([2H])c([2H])c4c23)c1[2H] ; [2H]c1c([2H])c(N(c2ccc(-c3ccccc3)cc2)c2cc3c4ccccc4n(-c4ccccc4)c3c3c2ccc2ccccc23)c([2H])c([2H])c1-c1c([2H])c([2H])c2c([2H])c([2H])c([2H])c([2H])c2c1[2H]]

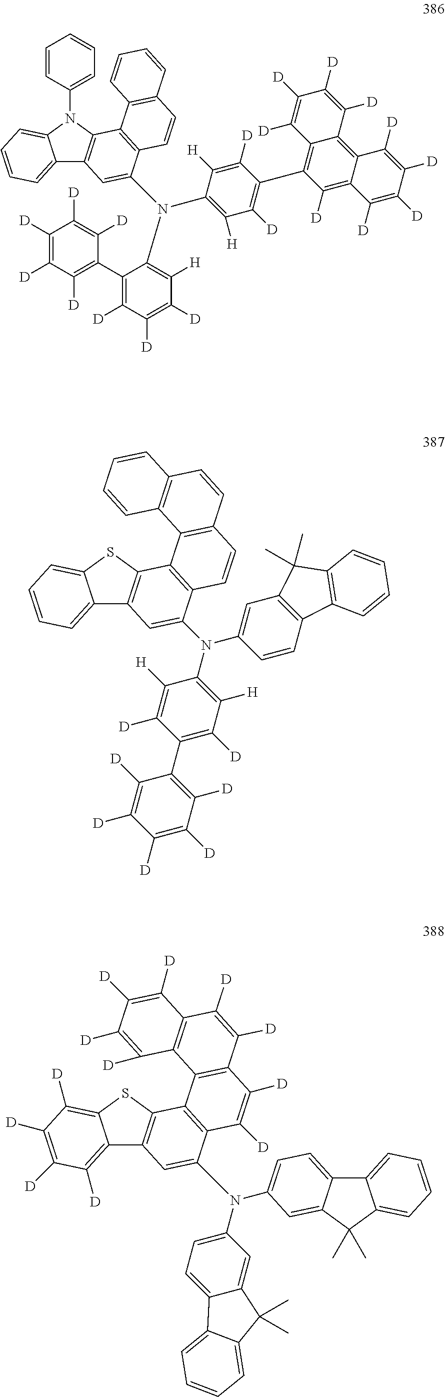

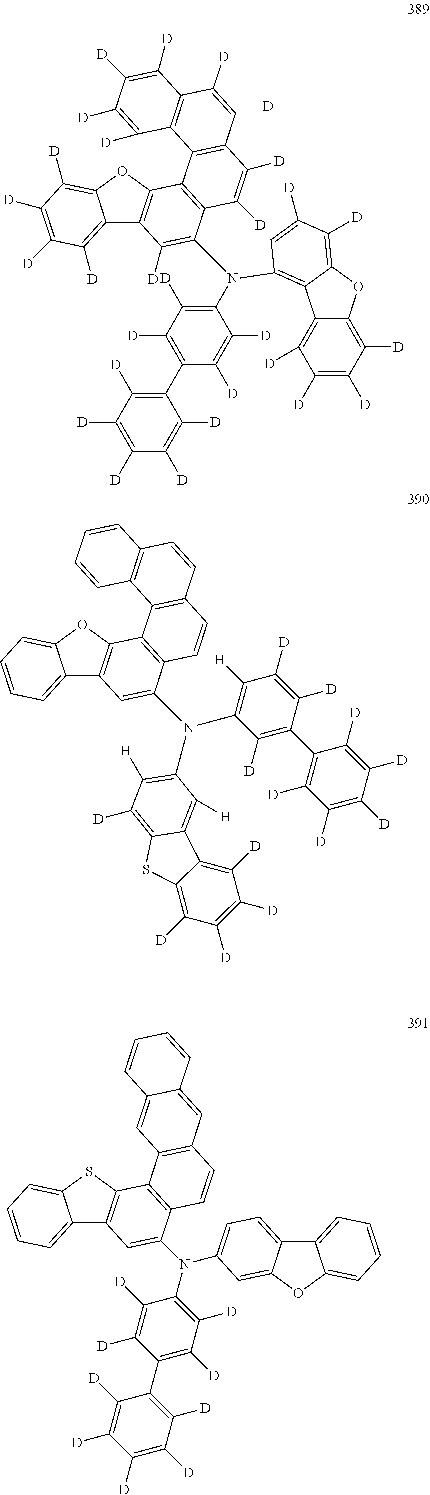

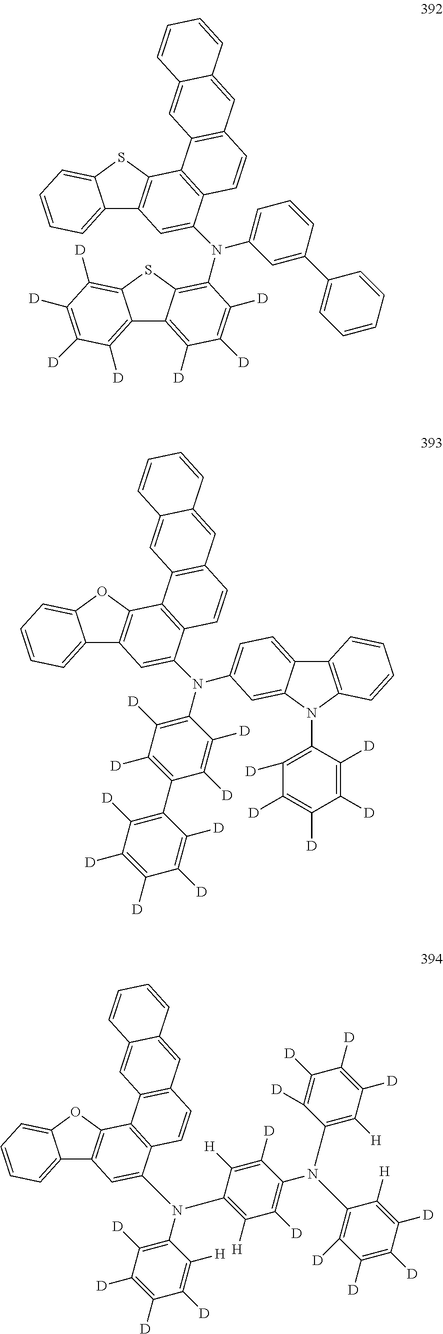 [SMILES: [2H]c1c([2H])c([2H])c2c(sc3c(N(c4cccc(-c5ccccc5)c4)c4cc5c6ccccc6sc5c5c4ccc4cc6ccccc6cc45)c([2H])c([2H])c([2H])c32)c1[2H] ; [2H]c1c([2H])c([2H])c(-c2c([2H])c([2H])c(N(c3ccc4c5ccccc5n(-c5c([2H])c([2H])c([2H])c([2H])c5[2H])c4c3)c3cc4c5ccccc5oc4c4c3ccc3cc5ccccc5cc34)c([2H])c2[2H])c([2H])c1[2H] ; [2H]c1cc(N(c2cc([2H])c([2H])c([2H])c2[2H])c2c([2H])cc(N(c3cc([2H])c([2H])c([2H])c3[2H])c3cc4c5ccccc5oc4c4c3ccc3cc5ccccc5cc34)cc2[2H])c([2H])c([2H])c1[2H]]

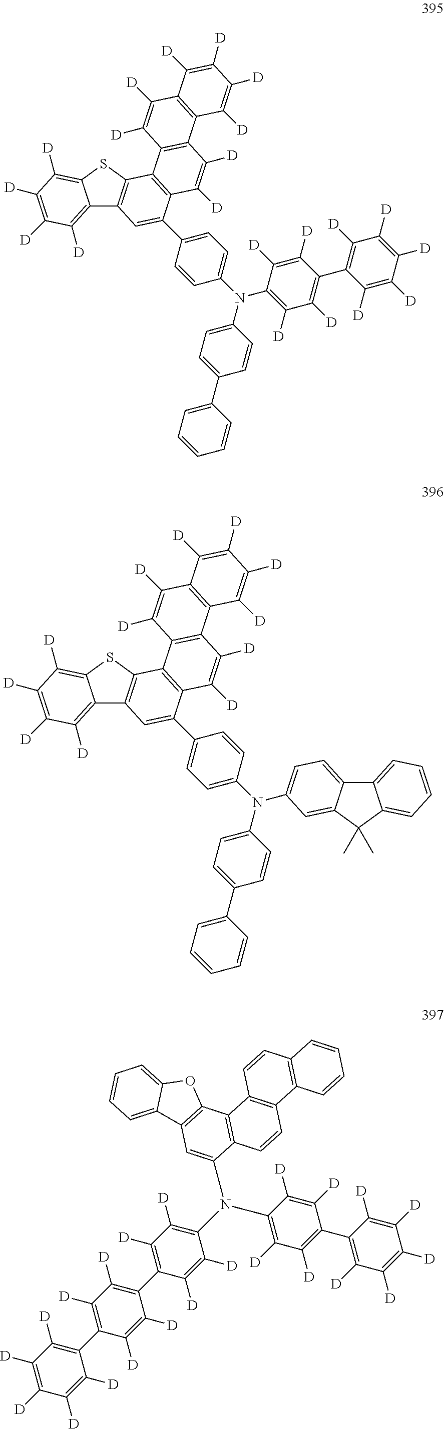

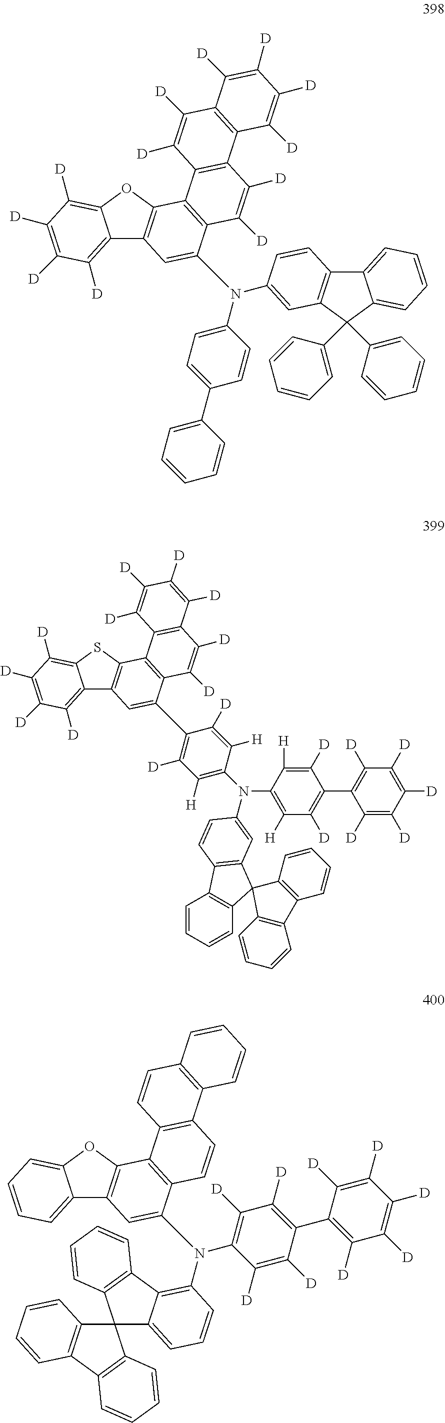

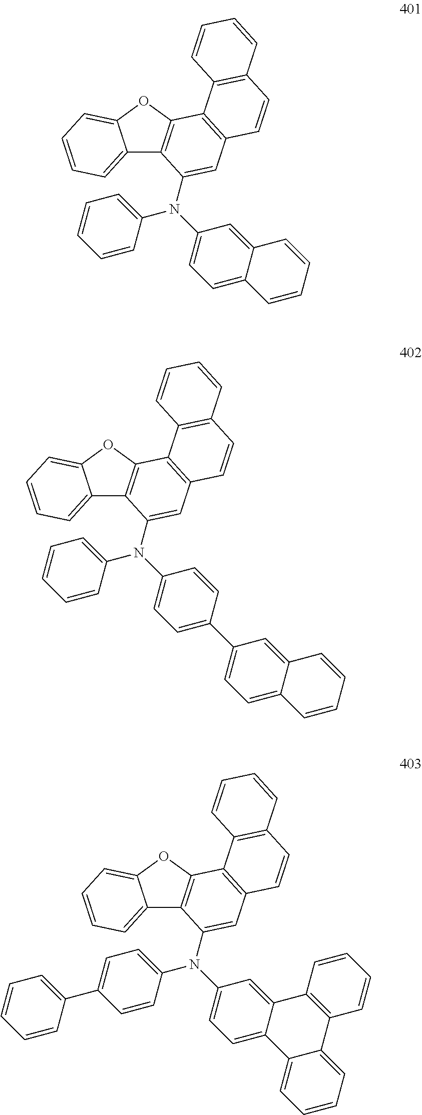 [SMILES: c1ccc(N(c2ccc3ccccc3c2)c2cc3ccc4ccccc4c3c3oc4ccccc4c23)cc1 ; c1ccc(N(c2ccc(-c3ccc4ccccc4c3)cc2)c2cc3ccc4ccccc4c3c3oc4ccccc4c23)cc1 ; c1ccc(-c2ccc(N(c3ccc4c5ccccc5c5ccccc5c4c3)c3cc4ccc5ccccc5c4c4oc5ccccc5c34)cc2)cc1]

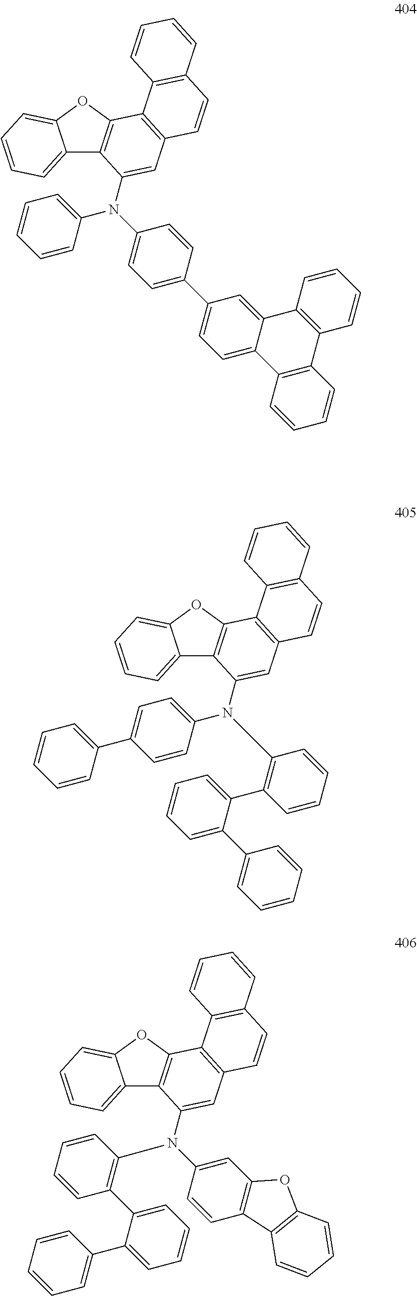 [SMILES: c1ccc(N(c2ccc(-c3ccc4c5ccccc5c5ccccc5c4c3)cc2)c2cc3ccc4ccccc4c3c3oc4ccccc4c23)cc1 ; c1ccc(-c2ccc(N(c3ccccc3-c3ccccc3-c3ccccc3)c3cc4ccc5ccccc5c4c4oc5ccccc5c34)cc2)cc1 ; c1ccc(-c2ccccc2-c2ccccc2N(c2ccc3c(c2)oc2ccccc23)c2cc3ccc4ccccc4c3c3oc4ccccc4c23)cc1]

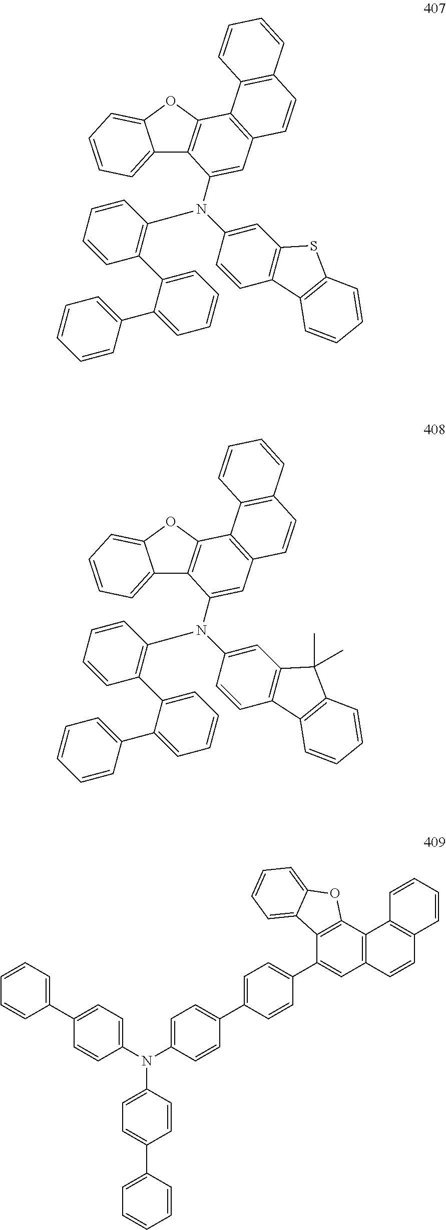 [SMILES: c1ccc(-c2ccccc2-c2ccccc2N(c2ccc3c(c2)sc2ccccc23)c2cc3ccc4ccccc4c3c3oc4ccccc4c23)cc1 ; CC1(C)c2ccccc2-c2ccc(N(c3ccccc3-c3ccccc3-c3ccccc3)c3cc4ccc5ccccc5c4c4oc5ccccc5c34)cc21 ; c1ccc(-c2ccc(N(c3ccc(-c4ccccc4)cc3)c3ccc(-c4ccc(-c5cc6ccc7ccccc7c6c6oc7ccccc7c56)cc4)cc3)cc2)cc1]

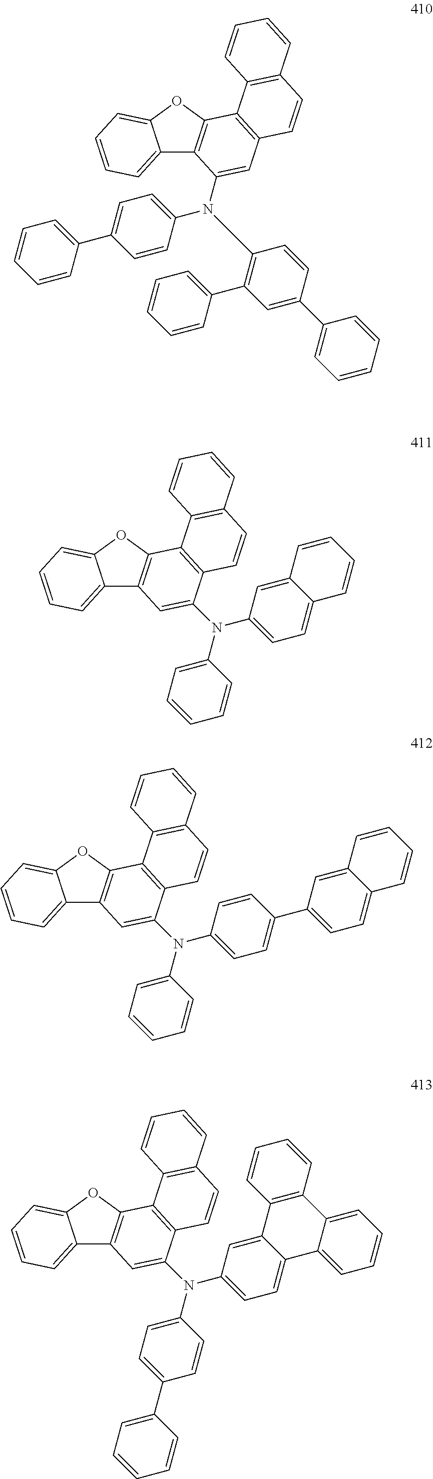 [SMILES: c1ccc(-c2ccc(N(c3ccc(-c4ccccc4)cc3-c3ccccc3)c3cc4ccc5ccccc5c4c4oc5ccccc5c34)cc2)cc1 ; c1ccc(N(c2ccc3ccccc3c2)c2cc3c4ccccc4oc3c3c2ccc2ccccc23)cc1 ; c1ccc(N(c2ccc(-c3ccc4ccccc4c3)cc2)c2cc3c4ccccc4oc3c3c2ccc2ccccc23)cc1 ; c1ccc(-c2ccc(N(c3ccc4c5ccccc5c5ccccc5c4c3)c3cc4c5ccccc5oc4c4c3ccc3ccccc34)cc2)cc1]

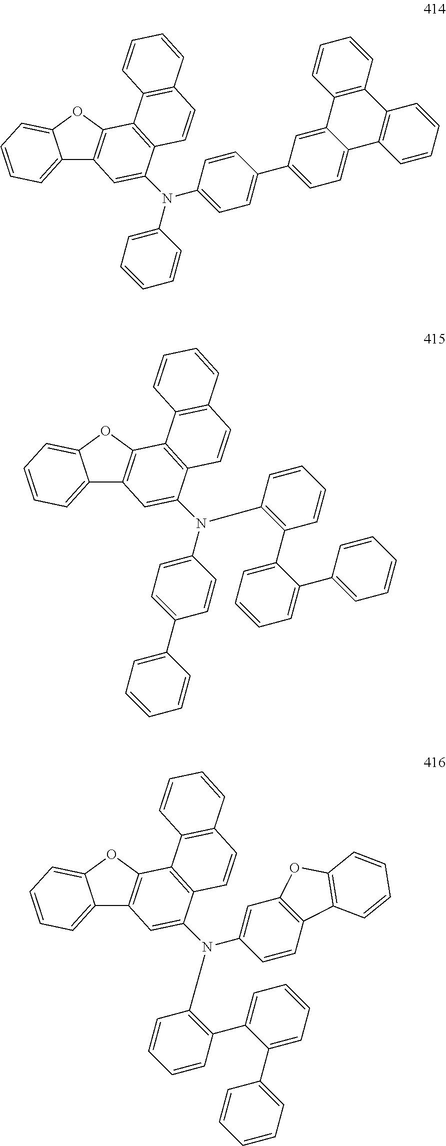 [SMILES: c1ccc(N(c2ccc(-c3ccc4c5ccccc5c5ccccc5c4c3)cc2)c2cc3c4ccccc4oc3c3c2ccc2ccccc23)cc1 ; c1ccc(-c2ccc(N(c3ccccc3-c3ccccc3-c3ccccc3)c3cc4c5ccccc5oc4c4c3ccc3ccccc34)cc2)cc1 ; c1ccc(-c2ccccc2-c2ccccc2N(c2ccc3c(c2)oc2ccccc23)c2cc3c4ccccc4oc3c3c2ccc2ccccc23)cc1]

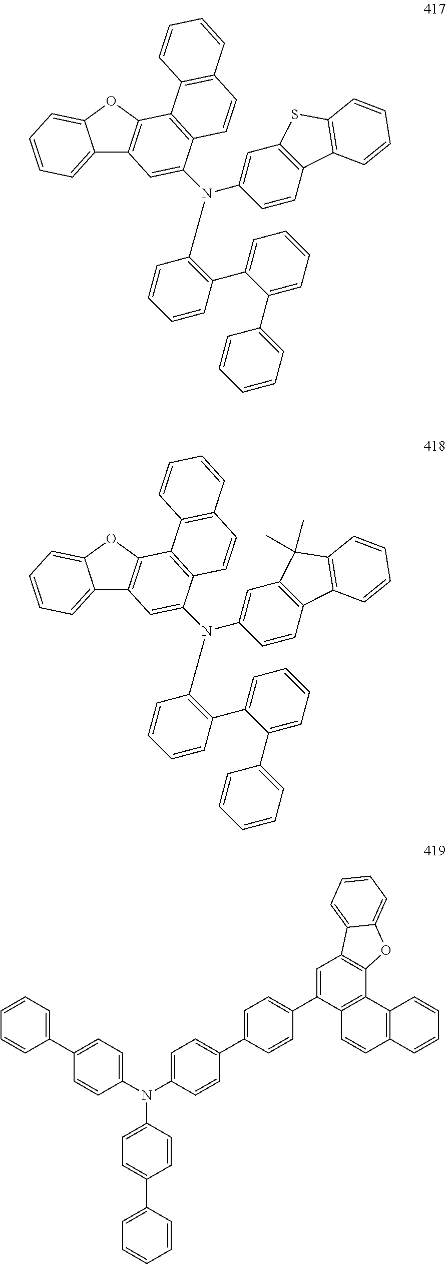 [SMILES: c1ccc(-c2ccccc2-c2ccccc2N(c2ccc3c(c2)sc2ccccc23)c2cc3c4ccccc4oc3c3c2ccc2ccccc23)cc1 ; CC1(C)c2ccccc2-c2ccc(N(c3ccccc3-c3ccccc3-c3ccccc3)c3cc4c5ccccc5oc4c4c3ccc3ccccc34)cc21 ; c1ccc(-c2ccc(N(c3ccc(-c4ccccc4)cc3)c3ccc(-c4ccc(-c5cc6c7ccccc7oc6c6c5ccc5ccccc56)cc4)cc3)cc2)cc1]

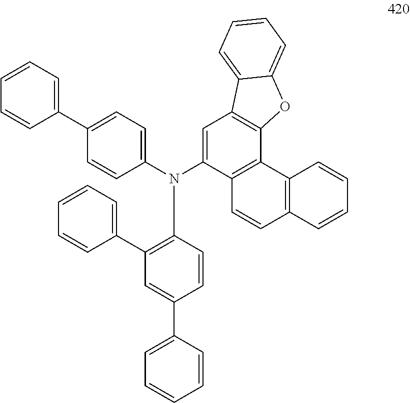 [SMILES: c1ccc(-c2ccc(N(c3ccc(-c4ccccc4)cc3-c3ccccc3)c3cc4c5ccccc5oc4c4c3ccc3ccccc34)cc2)cc1]

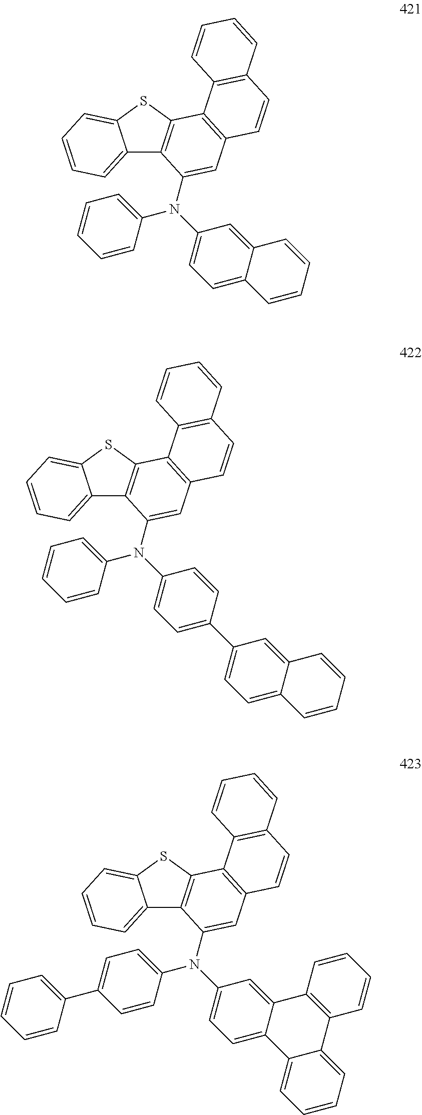 [SMILES: c1ccc(N(c2ccc3ccccc3c2)c2cc3ccc4ccccc4c3c3sc4ccccc4c23)cc1 ; c1ccc(N(c2ccc(-c3ccc4ccccc4c3)cc2)c2cc3ccc4ccccc4c3c3sc4ccccc4c23)cc1 ; c1ccc(-c2ccc(N(c3ccc4c5ccccc5c5ccccc5c4c3)c3cc4ccc5ccccc5c4c4sc5ccccc5c34)cc2)cc1]

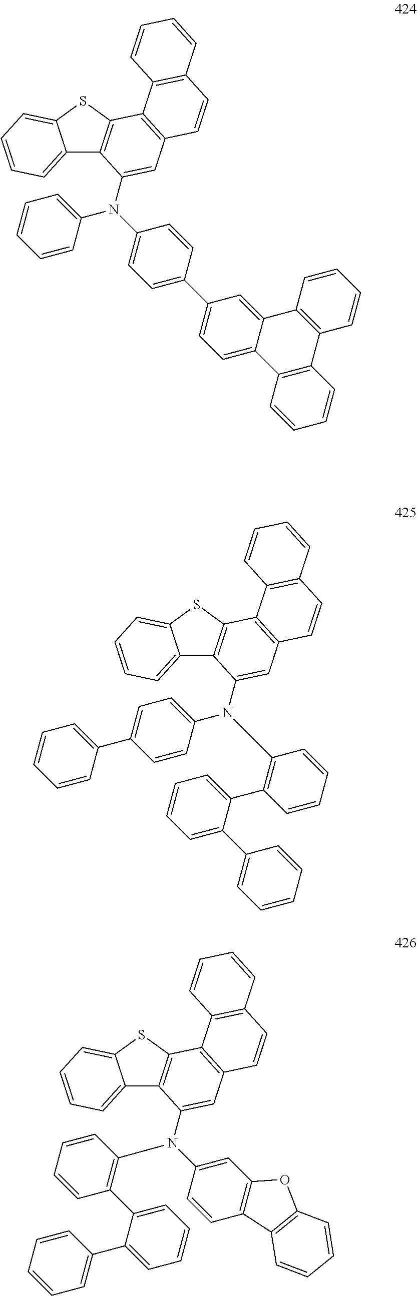 [SMILES: c1ccc(N(c2ccc(-c3ccc4c5ccccc5c5ccccc5c4c3)cc2)c2cc3ccc4ccccc4c3c3sc4ccccc4c23)cc1 ; c1ccc(-c2ccc(N(c3ccccc3-c3ccccc3-c3ccccc3)c3cc4ccc5ccccc5c4c4sc5ccccc5c34)cc2)cc1 ; c1ccc(-c2ccccc2-c2ccccc2N(c2ccc3c(c2)oc2ccccc23)c2cc3ccc4ccccc4c3c3sc4ccccc4c23)cc1]

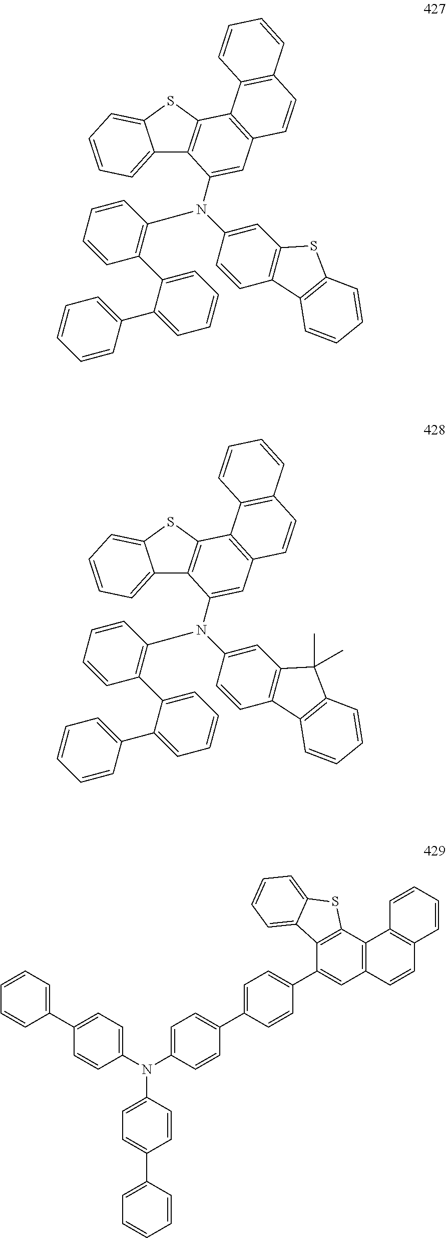 [SMILES: c1ccc(-c2ccccc2-c2ccccc2N(c2ccc3c(c2)sc2ccccc23)c2cc3ccc4ccccc4c3c3sc4ccccc4c23)cc1 ; CC1(C)c2ccccc2-c2ccc(N(c3ccccc3-c3ccccc3-c3ccccc3)c3cc4ccc5ccccc5c4c4sc5ccccc5c34)cc21 ; c1ccc(-c2ccc(N(c3ccc(-c4ccccc4)cc3)c3ccc(-c4ccc(-c5cc6ccc7ccccc7c6c6sc7ccccc7c56)cc4)cc3)cc2)cc1]

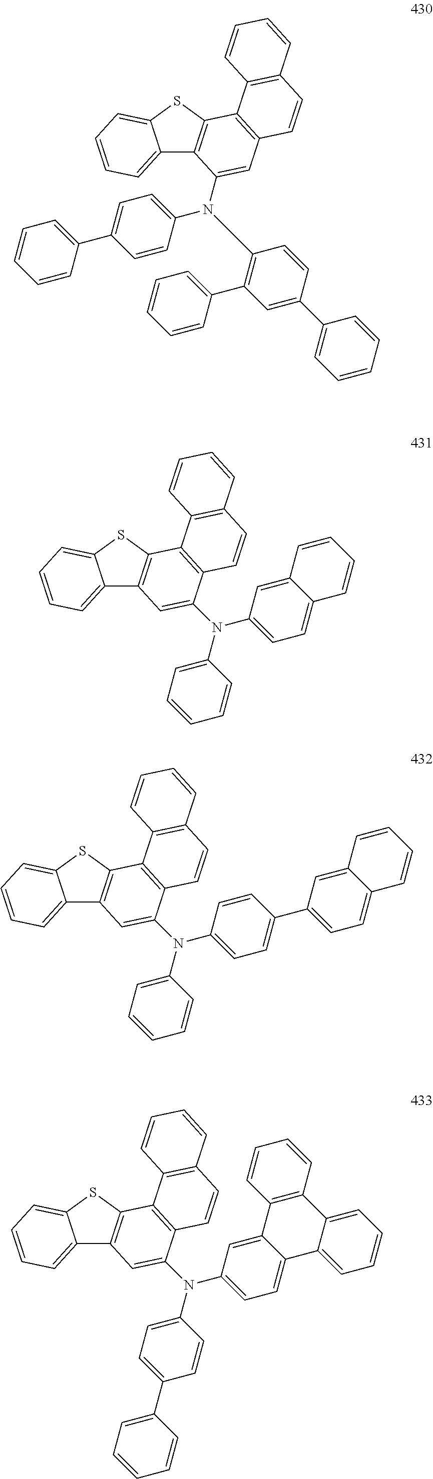 [SMILES: c1ccc(-c2ccc(N(c3ccc(-c4ccccc4)cc3-c3ccccc3)c3cc4ccc5ccccc5c4c4sc5ccccc5c34)cc2)cc1 ; c1ccc(N(c2ccc3ccccc3c2)c2cc3c4ccccc4sc3c3c2ccc2ccccc23)cc1 ; c1ccc(N(c2ccc(-c3ccc4ccccc4c3)cc2)c2cc3c4ccccc4sc3c3c2ccc2ccccc23)cc1 ; c1ccc(-c2ccc(N(c3ccc4c5ccccc5c5ccccc5c4c3)c3cc4c5ccccc5sc4c4c3ccc3ccccc34)cc2)cc1]

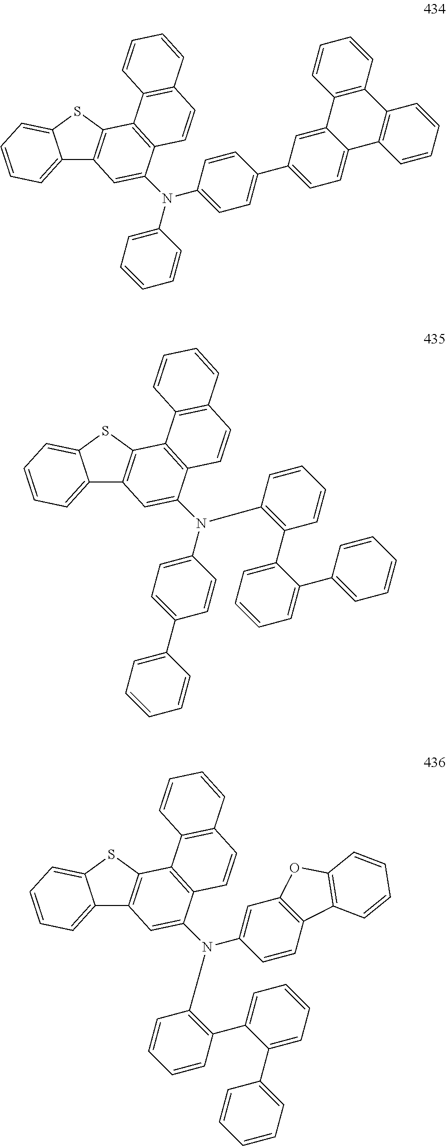 [SMILES: c1ccc(N(c2ccc(-c3ccc4c5ccccc5c5ccccc5c4c3)cc2)c2cc3c4ccccc4sc3c3c2ccc2ccccc23)cc1 ; c1ccc(-c2ccc(N(c3ccccc3-c3ccccc3-c3ccccc3)c3cc4c5ccccc5sc4c4c3ccc3ccccc34)cc2)cc1 ; c1ccc(-c2ccccc2-c2ccccc2N(c2ccc3c(c2)oc2ccccc23)c2cc3c4ccccc4sc3c3c2ccc2ccccc23)cc1]

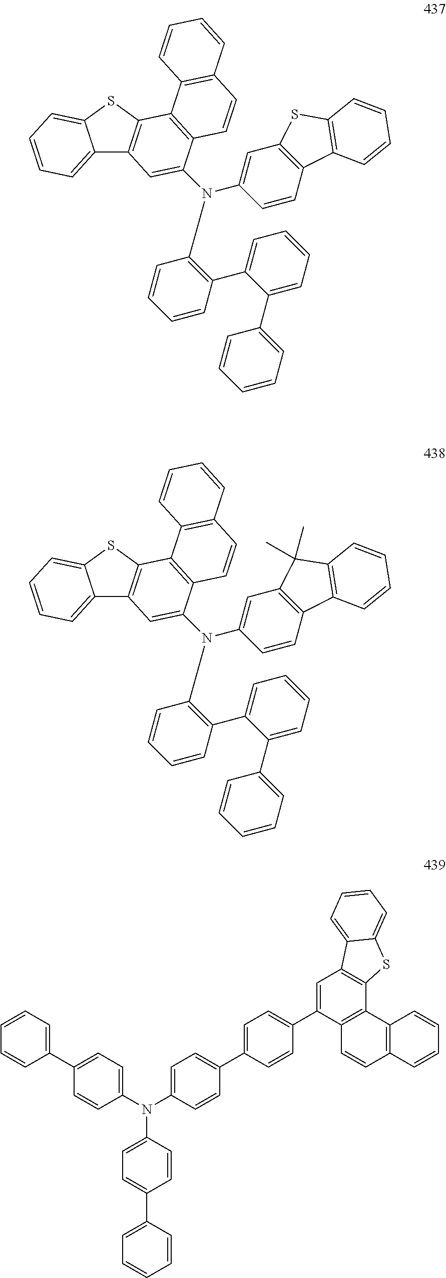 [SMILES: c1ccc(-c2ccccc2-c2ccccc2N(c2ccc3c(c2)sc2ccccc23)c2cc3c4ccccc4sc3c3c2ccc2ccccc23)cc1 ; CC1(C)c2ccccc2-c2ccc(N(c3ccccc3-c3ccccc3-c3ccccc3)c3cc4c5ccccc5sc4c4c3ccc3ccccc34)cc21 ; c1ccc(-c2ccc(N(c3ccc(-c4ccccc4)cc3)c3ccc(-c4ccc(-c5cc6c7ccccc7sc6c6c5ccc5ccccc56)cc4)cc3)cc2)cc1]

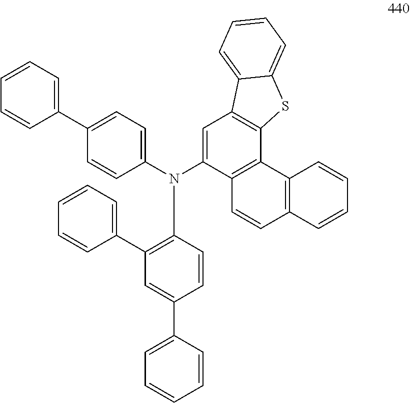 [SMILES: c1ccc(-c2ccc(N(c3ccc(-c4ccccc4)cc3-c3ccccc3)c3cc4c5ccccc5sc4c4c3ccc3ccccc34)cc2)cc1]

In addition, by introducing various substituents to the structure of Chemical Formula 1, compounds having unique properties of the introduced substituents may be synthesized. For example, by introducing substituents normally used for a hole injection layer material, a hole transport layer material, a light emitting layer material, an electron transport layer material, an electron blocking layer material and a charge generation layer material used for manufacturing an organic light emitting device to the core structure, materials satisfying conditions required for each organic material layer may be synthesized.

In addition, by introducing various substituents to the structure of Chemical Formula 1, the energy band gap may be finely controlled, and meanwhile, properties at interfaces between organic materials may be enhanced, and material applications may become diverse.

Meanwhile, the heterocyclic compound has a high glass transition temperature (Tg), and thereby has excellent thermal stability. Such an increase in the thermal stability becomes an important factor providing driving stability to a device.

The heterocyclic compound according to one embodiment of the present disclosure may be prepared using a multi-step chemical reaction. Some intermediate compounds are prepared first, and from the intermediate compounds, the compound of Chemical Formula 1 may be prepared. More specifically, the heterocyclic compound according to one embodiment of the present disclosure may be prepared based on preparation examples to be described later.

Another embodiment of the present disclosure provides an organic light emitting device including the heterocyclic compound represented by Chemical Formula 1. The “organic light emitting device” may be expressed in terms such as an “organic light emitting diode”, an “OLED”, an “OLED device” and an “organic electroluminescent device”.

In addition, one embodiment of the present disclosure relates to an organic light emitting device comprising: a first electrode; a second electrode provided opposite to the first electrode; and one or more organic material layers provided between the first electrode and the second electrode, wherein one or more layers of the one or more organic material layers comprise the heterocyclic compound represented by Chemical Formula 1.

In one embodiment of the present disclosure, the first electrode may be a positive electrode, and the second electrode may be a negative electrode.

In another embodiment, the first electrode may be a negative electrode, and the second electrode may be a positive electrode.

In one embodiment of the present disclosure, the organic material layer may include one or more selected from the group consisting of an electron injection layer, an electron transport layer, a hole blocking layer, a light emitting layer, a light emitting auxiliary layer, an electron blocking layer, a hole transport layer and a hole injection layer, and the one or more layers selected from the group consisting of an electron injection layer, an electron transport layer, a hole blocking layer, a light emitting layer, a light emitting auxiliary layer, an electron blocking layer, a hole transport layer and a hole injection layer may include the heterocyclic compound represented by Chemical Formula 1.

In another embodiment of the present disclosure, the organic material layer may include an electron injection layer or an electron transport layer, and the electron injection layer or the electron transport layer may include the heterocyclic compound represented by Chemical Formula 1.

In another embodiment of the present disclosure, the organic material layer may include an electron blocking layer or a hole blocking layer, and the electron blocking layer or the hole blocking layer may include the heterocyclic compound represented by Chemical Formula 1.

In another embodiment of the present disclosure, the organic material layer may include an electron blocking layer, a light emitting layer or a hole transport layer, and the electron blocking layer, the light emitting layer or the hole transport layer may include the heterocyclic compound represented by Chemical Formula 1.

In another embodiment of the present disclosure, the organic material layer may include a hole transport layer, and the hole transport layer may include the heterocyclic compound represented by Chemical Formula 1. The heterocyclic compound represented by Chemical Formula 1 has fast hole mobility and appropriate HOMO level, and therefore, when the heterocyclic compound represented by Chemical Formula 1 is used in the hole transport layer, hole transport to the light emitting layer is facilitated, and as a result, a driving voltage of the organic light emitting device may be lowered, and driving efficiency and lifetime thereof may be improved.

In another embodiment of the present disclosure, the organic material layer may include an electron blocking layer, and the electron blocking layer may include the heterocyclic compound represented by Chemical Formula 1. The heterocyclic compound represented by Chemical Formula 1 has a high LUMO level, and therefore, when the heterocyclic compound represented by Chemical Formula 1 is used in the electron blocking layer, probability of holes and electrons forming excitons may increase, which may increase the probability of the excitons being emitted as light in the light emitting layer. Accordingly, an electron blocking ability is improved, and charge balance between holes and electrons is achieved, and as a result, driving efficiency and lifetime of the organic light emitting device may become superior.

In another embodiment of the present disclosure, the organic material layer may include a light emitting layer, and the light emitting layer may include the heterocyclic compound represented by Chemical Formula 1. When the heterocyclic compound represented by Chemical Formula 1 is used in the light emitting layer, HOMO (Highest Occupied Molecular Orbital) and LUMO (Lowest Unoccupied Molecular Orbital) are spatially separated, enabling strong charge transfer, and therefore, driving efficiency and lifetime of the organic light emitting device may become superior.

In another embodiment of the present disclosure, the organic material layer includes a light emitting layer, the light emitting layer includes a host material, and the host material may include the heterocyclic compound represented by Chemical Formula 1.

In one embodiment of the present disclosure, the organic light emitting device may be a red organic light emitting device, and the heterocyclic compound represented by Chemical Formula 1 may be used as a material of the red organic light emitting device.

In another embodiment of the present disclosure, the organic light emitting device may be a blue organic light emitting device, and the heterocyclic compound represented by Chemical Formula 1 may be used as a material of the blue organic light emitting device.

In another embodiment of the present disclosure, the organic light emitting device may be a green organic light emitting device, and the heterocyclic compound represented by Chemical Formula 1 may be used as a material of the green organic light emitting device.

In one embodiment of the present disclosure, the organic light emitting device may be a red organic light emitting device, and the heterocyclic compound represented by Chemical Formula 1 may be used as a light emitting layer material of the red organic light emitting device.

In another embodiment of the present disclosure, the organic light emitting device may be a blue organic light emitting device, and the heterocyclic compound represented by Chemical Formula 1 may be used as a light emitting layer material of the blue organic light emitting device.

In another embodiment of the present disclosure, the organic light emitting device may be a green organic light emitting device, and the heterocyclic compound represented by Chemical Formula 1 may be used as a light emitting layer material of the green organic light emitting device.

In one embodiment of the present disclosure, the organic light emitting device may further include one, or two or more layers selected from the group consisting of a light emitting layer, a hole injection layer, a hole transport layer, an electron injection layer, an electron transport layer, an electron blocking layer and a hole blocking layer.

Specific descriptions on the heterocyclic compound represented by Chemical Formula 1 are the same as the descriptions provided above.

The organic light emitting device of the present disclosure may be manufactured using common organic light emitting device manufacturing methods and materials except that one or more organic material layers are formed using the heterocyclic compound represented by Chemical Formula 1.

The heterocyclic compound may be formed into the organic material layer using a solution coating method as well as a vacuum deposition method when the organic light emitting device is manufactured. Herein, the solution coating method means spin coating, dip coating, inkjet printing, screen printing, a spray method, roll coating and the like, but is not limited thereto.

The organic material layer of the organic light emitting device of the present disclosure may be formed in a single layer structure, but may also be formed in a multilayer structure in which two or more organic material layers are laminated. For example, the organic light emitting device of the present disclosure may have a structure including a hole injection layer, an electron blocking layer, a hole transport layer, a light emitting layer, an electron transport layer, a hole blocking layer, an electron injection layer and the like as the organic material layer. However, the structure of the organic light emitting device is not limited thereto, and may include a smaller number of organic material layers.

One embodiment of the present disclosure provides an organic light emitting device, wherein the organic material layer including the heterocyclic compound represented by Chemical Formula 1 further includes a heterocyclic compound represented by the following Chemical Formula 2.

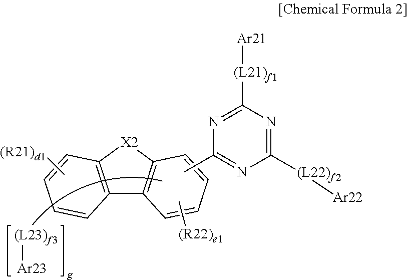

In Chemical Formula 2,
X2 is O; or S,
R21 and R22 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; —P(═O)R201R202; —SiR201R202R203; and —NR201R202, or two or more groups adjacent to each other bond to each other to form a substituted or unsubstituted C6 to C60 aromatic hydrocarbon ring; or a substituted or unsubstituted C2 to C60 heteroring, and R201, R202 and R203 are the same as or different from each other and each independently a substituted or unsubstituted C1 to C60 alkyl group; a substituted or unsubstituted C6 to C60 aryl group; or a substituted or unsubstituted C2 to C60 heteroaryl group,
d1 is an integer of 0 to 4, and when d1 is 2 or greater, R21s are the same as or different from each other,
e1 is an integer of 0 to 3, and when e1 is 2 or greater, R22s are the same as or different from each other,
L21 to L23 are the same as or different from each other, and each independently a direct bond; a substituted or unsubstituted C6 to C60 arylene group; or a substituted or unsubstituted C2 to C60 heteroarylene group,
f1 is an integer of 0 to 5, and when f1 is 2 or greater, L21s are the same as or different from each other,
f2 is an integer of 0 to 5, and when f2 is 2 or greater, L22s are the same as or different from each other,
f3 is an integer of 0 to 5, and when f3 is 2 or greater, L23s are the same as or different from each other,
Ar21 to Ar23 are the same as or different from each other, and each independently a substituted or unsubstituted C6 to C60 aryl group; a substituted or unsubstituted C2 to C60 heteroaryl group; or —NAr24Ar25,
Ar24 and Ar25 are the same as or different from each other, and each independently a substituted or unsubstituted C6 to C60 aryl group; or a substituted or unsubstituted C2 to C60 heteroaryl group, and
g is 0 or 1.

In one embodiment of the present disclosure, X2 may be O.

In another embodiment of the present disclosure, X2 may be S.

In one embodiment of the present disclosure, R21 and R22 are the same as or different from each other, and each independently hydrogen; deuterium; halogen; a cyano group; a substituted or unsubstituted C1 to C30 alkyl group; a substituted or unsubstituted C2 to C30 alkenyl group; a substituted or unsubstituted C2 to C30 alkynyl group; a substituted or unsubstituted C1 to C30 alkoxy group; a substituted or unsubstituted C3 to C30 cycloalkyl group; a substituted or unsubstituted C2 to C30 heterocycloalkyl group; —P(═O)R201R202; —SiR201R202R203; or —NR201R202, or two or more groups adjacent to each other bond to each other to form a substituted or unsubstituted C6 to C30 aromatic hydrocarbon ring; or a substituted or unsubstituted C2 to C30 heteroring, and R201, R202 and R203 are the same as or different from each other and may be each independently a substituted or unsubstituted C1 to C30 alkyl group; a substituted or unsubstituted C6 to C30 aryl group; or a substituted or unsubstituted C2 to C30 heteroaryl group.

In another embodiment of the present disclosure, R21 and R22 are the same as or different from each other, and each independently hydrogen; deuterium; halogen; a cyano group; a substituted or unsubstituted C1 to C20 alkyl group; a substituted or unsubstituted C2 to C20 alkenyl group; a substituted or unsubstituted C2 to C20 alkynyl group; a substituted or unsubstituted C1 to C20 alkoxy group; a substituted or unsubstituted C3 to C20 cycloalkyl group; a substituted or unsubstituted C2 to C20 heterocycloalkyl group; —P(═O)R201R202; —SiR201R202R203; or —NR201R202, or two or more groups adjacent to each other bond to each other to form a substituted or unsubstituted C6 to C20 aromatic hydrocarbon ring; or a substituted or unsubstituted C2 to C20 heteroring, and R201, R202 and R203 are the same as or different from each other and may be each independently a substituted or unsubstituted C1 to C20 alkyl group; a substituted or unsubstituted C6 to C20 aryl group; or a substituted or unsubstituted C2 to C20 heteroaryl group.

In another embodiment of the present disclosure, R21 and R22 are the same as or different from each other, and each independently hydrogen; deuterium, or two or more groups adjacent to each other may bond to each other to form a substituted or unsubstituted C6 to C20 aromatic hydrocarbon ring; or a substituted or unsubstituted C2 to C20 heteroring.

In another embodiment of the present disclosure, R21 and R22 are the same as or different from each other, and each independently hydrogen; or deuterium, or two or more groups adjacent to each other may bond to each other to form a substituted or unsubstituted C6 to C20 aromatic hydrocarbon ring.

In one embodiment of the present disclosure, L21 to L23 are the same as or different from each other, and may be each independently a direct bond; a substituted or unsubstituted C6 to C30 arylene group; or a substituted or unsubstituted C2 to C30 heteroarylene group.

In another embodiment of the present disclosure, L21 to L23 are the same as or different from each other, and may be each independently a direct bond; a substituted or unsubstituted C6 to C20 arylene group; or a substituted or unsubstituted C2 to C20 heteroarylene group.

In another embodiment of the present disclosure, L21 to L23 are the same as or different from each other, and may be each independently a direct bond; or a substituted or unsubstituted C6 to C60 arylene group.

In another embodiment of the present disclosure, L21 to L23 are the same as or different from each other, and may be each independently a direct bond; or a substituted or unsubstituted C6 to C30 arylene group.

In another embodiment of the present disclosure, L21 to L23 are the same as or different from each other, and may be each independently a direct bond; or a substituted or unsubstituted C6 to C20 arylene group.

In another embodiment of the present disclosure, L21 to L23 are the same as or different from each other, and may be each independently a direct bond; a substituted or unsubstituted phenylene group; a substituted or unsubstituted biphenylene group; a substituted or unsubstituted naphthylene group; a substituted or unsubstituted phenanthrenylene group; or a substituted or unsubstituted chrysenylene group.

In another embodiment of the present disclosure, L21 and L22 are the same as or different from each other, and may be each independently a direct bond; a substituted or unsubstituted phenylene group; a substituted or unsubstituted naphthylene group; or a substituted or unsubstituted chrysenylene group.

In another embodiment of the present disclosure, L23 may be a direct bond; a substituted or unsubstituted phenylene group; a substituted or unsubstituted biphenylene group; a substituted or unsubstituted naphthylene group; or a substituted or unsubstituted phenanthrenylene group.

In one embodiment of the present disclosure, Ar21 to Ar23 are the same as or different from each other, and may be each independently a substituted or unsubstituted C6 to C30 aryl group; a substituted or unsubstituted C2 to C30 heteroaryl group; or —NAr24Ar25.

In another embodiment of the present disclosure, Ar21 to Ar23 are the same as or different from each other, and may be each independently a substituted or unsubstituted C6 to C20 aryl group; a substituted or unsubstituted C2 to C20 heteroaryl group; or —NAr24Ar25.

In another embodiment of the present disclosure, Ar21 and Ar22 are the same as or different from each other, and may be each independently a substituted or unsubstituted C6 to C30 aryl group; or a substituted or unsubstituted C2 to C30 heteroaryl group.

In another embodiment of the present disclosure, Ar21 and Ar22 are the same as or different from each other, and may be each independently a substituted or unsubstituted C6 to C20 aryl group; or a substituted or unsubstituted C2 to C20 heteroaryl group.

In another embodiment of the present disclosure, Ar21 and Ar22 are the same as or different from each other, and may be each independently a substituted or unsubstituted phenyl group; a substituted or unsubstituted naphthyl group; a substituted or unsubstituted phenanthrenyl group; a substituted or unsubstituted dibenzofuranyl group; or a substituted or unsubstituted dibenzothiophenyl group.

In another embodiment of the present disclosure, Ar23 may be a substituted or unsubstituted phenyl group; a substituted or unsubstituted biphenyl group; a substituted or unsubstituted terphenyl group; a substituted or unsubstituted naphthyl group; a substituted or unsubstituted benzocarbazolyl group; or —NAr24Ar25.

In one embodiment of the present disclosure, Ar24 and Ar25 are the same as or different from each other, and may be each independently a substituted or unsubstituted C6 to C30 aryl group; or a substituted or unsubstituted C2 to C30 heteroaryl group.

In another embodiment of the present disclosure, Ar24 and Ar25 are the same as or different from each other, and may be each independently a substituted or unsubstituted C6 to C20 aryl group; or a substituted or unsubstituted C2 to C20 heteroaryl group.

In another embodiment of the present disclosure, Ar24 and Ar25 are the same as or different from each other, and may be each independently a substituted or unsubstituted C6 to C60 aryl group.

In another embodiment of the present disclosure, Ar24 and Ar25 are the same as or different from each other, and may be each independently a substituted or unsubstituted C6 to C30 aryl group.

In another embodiment of the present disclosure, Ar24 and Ar25 are the same as or different from each other, and may be each independently a substituted or unsubstituted C6 to C20 aryl group.

In another embodiment of the present disclosure. Ar24 and Ar25 may be a substituted or unsubstituted phenyl group; or a substituted or unsubstituted biphenyl group.

In one embodiment of the present disclosure, the heterocyclic compound represented by Chemical Formula 2 may be represented by any one of the following Chemical Formulae 2-1 to 2-3.

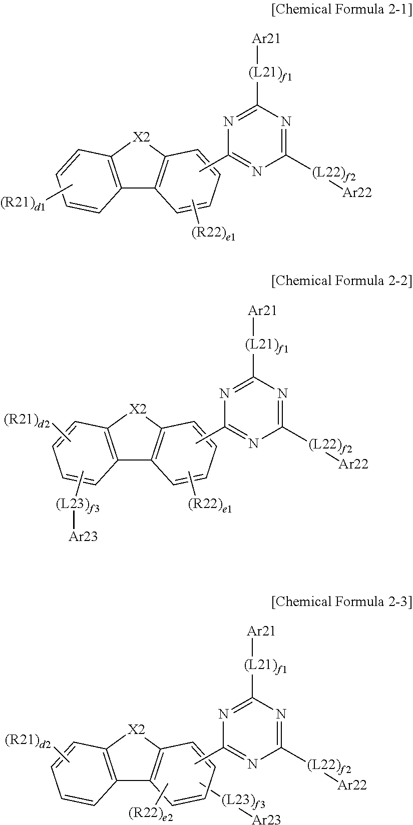

In Chemical Formulae 2-1 to 2-3,
d2 is an integer of 0 to 3, and when d2 is 2 or greater, R21s are the same as or different from each other,
e2 is an integer of 0 to 2, and when e2 is 2 or greater, R22s are the same as or different from each other, and
X2, R21, R22, L21 to L23, Ar21 to Ar23, d1, e1, and f1 to f3 have the same definitions as in Chemical Formula 2.

In one embodiment of the present disclosure, R21, R22, L21 to L23, and Ar21 to Ar25 may all include hydrogen (H) that is not deuterated.

In another embodiment of the present disclosure, at least one of R21, R22, L21 to L23, and Ar21 to Ar25 includes deuterium (D), and at least one of R21, R22, L21 to L23, and Ar21 to Ar25 may include hydrogen that is not deuterated.

In another embodiment of the present disclosure, R21, R22, L21 to L23, and Ar21 to Ar25 may all include deuterium.

In one embodiment of the present disclosure, the heterocyclic compound represented by Chemical Formula 2 may not include deuterium as a substituent, or may have a deuterium content of, for example, greater than 0%, 1% or greater, 10% or greater, 20% or greater, 30% or greater, 40% or greater or 50% or greater, and 100% or less, 90% or less, 80% or less, 70% or less or 60% or less with respect to the total number of hydrogen atoms and deuterium atoms.

In another embodiment of the present disclosure, the heterocyclic compound represented by Chemical Formula 2 may not include deuterium as a substituent, or may have a deuterium content of 1% to 100% with respect to the total number of hydrogen atoms and deuterium atoms.

In another embodiment of the present disclosure, the heterocyclic compound represented by Chemical Formula 2 may not include deuterium as a substituent, or may have a deuterium content of 20% to 90% with respect to the total number of hydrogen atoms and deuterium atoms.

In another embodiment of the present disclosure, the heterocyclic compound represented by Chemical Formula 2 may not include deuterium as a substituent, or may have a deuterium content of 30% to 80% with respect to the total number of hydrogen atoms and deuterium atoms.

In another embodiment of the present disclosure, the heterocyclic compound represented by Chemical Formula 2 may not include deuterium as a substituent, or may have a deuterium content of 50% to 70% with respect to the total number of hydrogen atoms and deuterium atoms.

In one embodiment of the present disclosure, the heterocyclic compound represented by Chemical Formula 2 may be represented by any one of the following compounds.

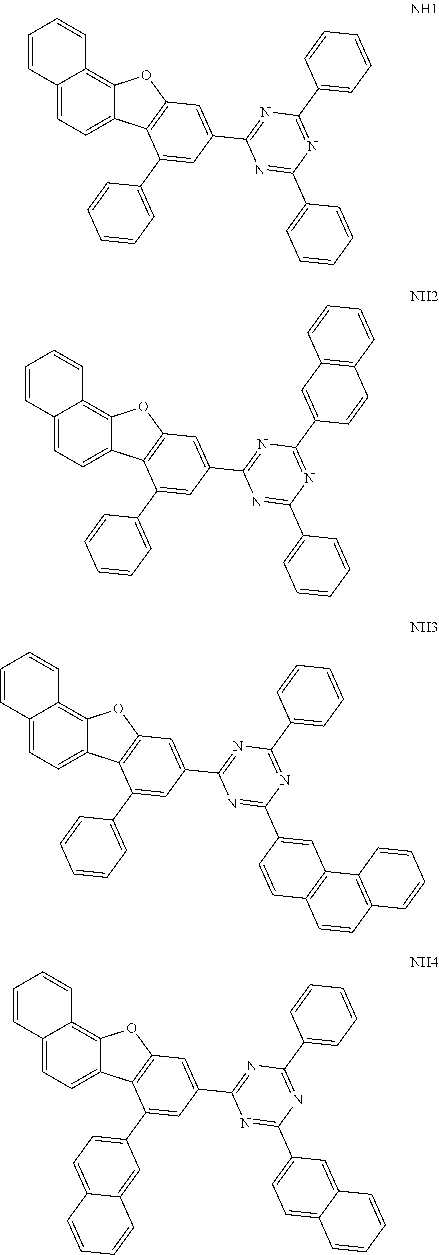

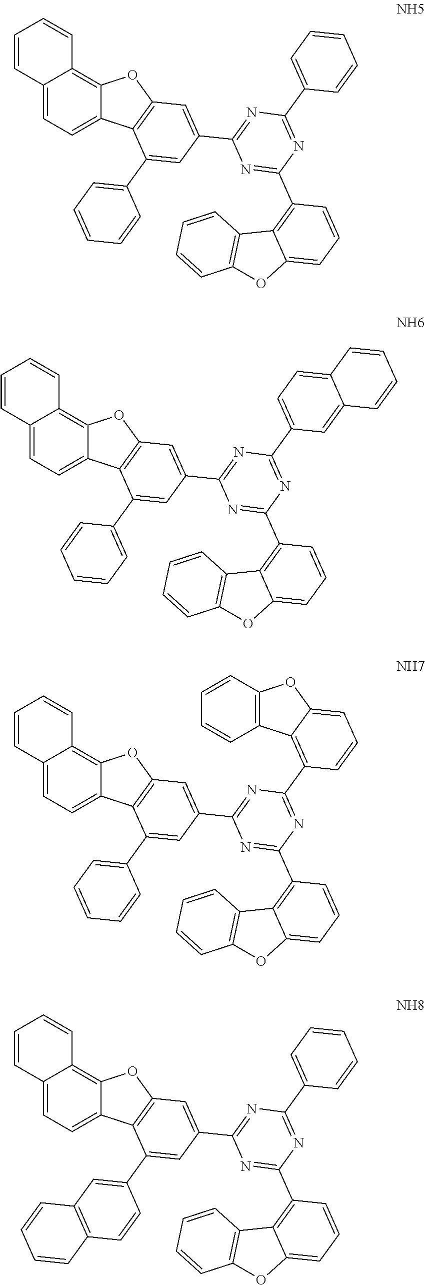 [SMILES: c1ccc(-c2nc(-c3cc(-c4ccccc4)c4c(c3)oc3c5ccccc5ccc34)nc(-c3cccc4oc5ccccc5c34)n2)cc1 ; c1ccc(-c2cc(-c3nc(-c4ccc5ccccc5c4)nc(-c4cccc5oc6ccccc6c45)n3)cc3oc4c5ccccc5ccc4c23)cc1 ; c1ccc(-c2cc(-c3nc(-c4cccc5oc6ccccc6c45)nc(-c4cccc5oc6ccccc6c45)n3)cc3oc4c5ccccc5ccc4c23)cc1 ; c1ccc(-c2nc(-c3cc(-c4ccc5ccccc5c4)c4c(c3)oc3c5ccccc5ccc34)nc(-c3cccc4oc5ccccc5c34)n2)cc1]

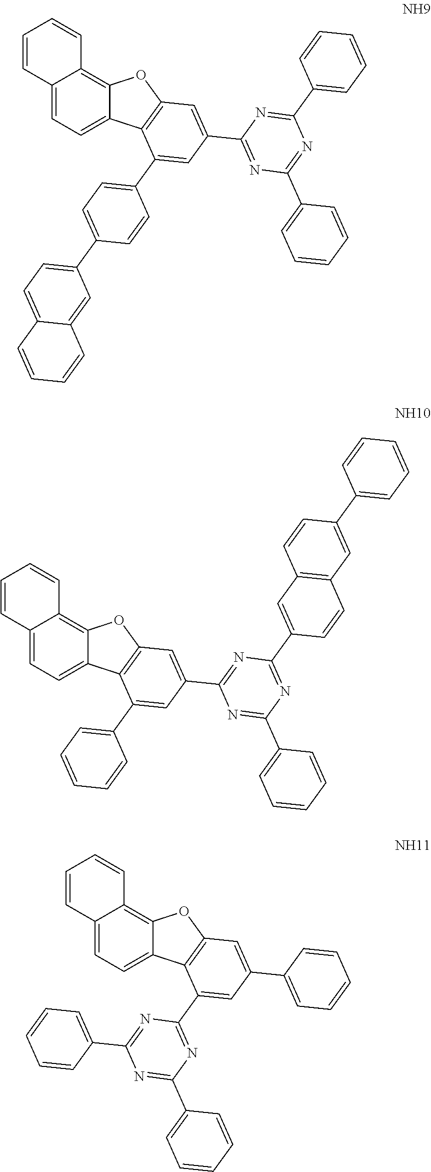 [SMILES: c1ccc(-c2nc(-c3ccccc3)nc(-c3cc(-c4ccc(-c5ccc6ccccc6c5)cc4)c4c(c3)oc3c5ccccc5ccc34)n2)cc1 ; c1ccc(-c2ccc3cc(-c4nc(-c5ccccc5)nc(-c5cc(-c6ccccc6)c6c(c5)oc5c7ccccc7ccc56)n4)ccc3c2)cc1 ; c1ccc(-c2cc(-c3nc(-c4ccccc4)nc(-c4ccccc4)n3)c3c(c2)oc2c4ccccc4ccc23)cc1]

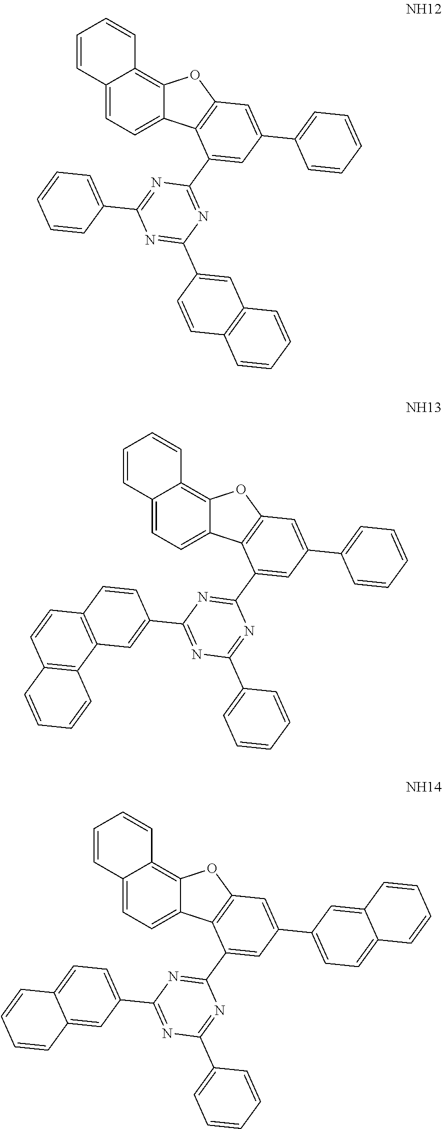 [SMILES: c1ccc(-c2cc(-c3nc(-c4ccccc4)nc(-c4ccc5ccccc5c4)n3)c3c(c2)oc2c4ccccc4ccc23)cc1 ; c1ccc(-c2cc(-c3nc(-c4ccccc4)nc(-c4ccc5ccc6ccccc6c5c4)n3)c3c(c2)oc2c4ccccc4ccc23)cc1 ; c1ccc(-c2nc(-c3ccc4ccccc4c3)nc(-c3cc(-c4ccc5ccccc5c4)cc4oc5c6ccccc6ccc5c34)n2)cc1]

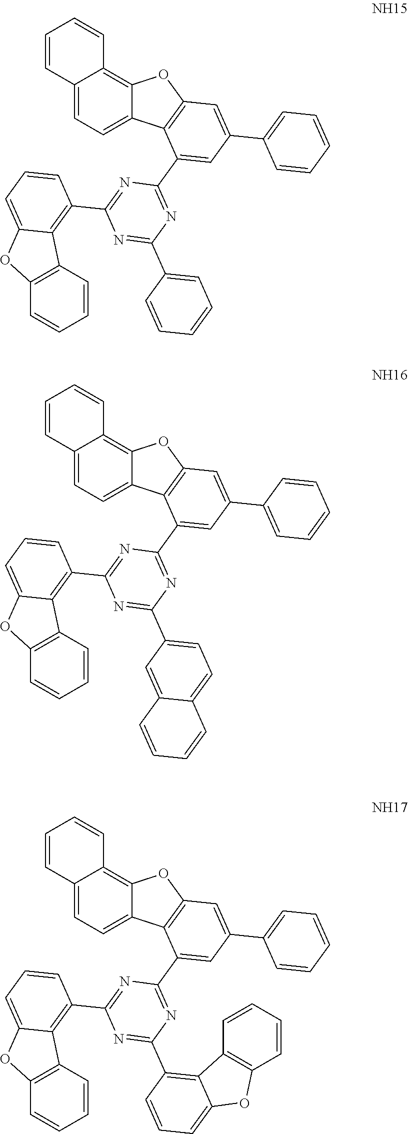 [SMILES: c1ccc(-c2cc(-c3nc(-c4ccccc4)nc(-c4cccc5oc6ccccc6c45)n3)c3c(c2)oc2c4ccccc4ccc23)cc1 ; c1ccc(-c2cc(-c3nc(-c4ccc5ccccc5c4)nc(-c4cccc5oc6ccccc6c45)n3)c3c(c2)oc2c4ccccc4ccc23)cc1 ; c1ccc(-c2cc(-c3nc(-c4cccc5oc6ccccc6c45)nc(-c4cccc5oc6ccccc6c45)n3)c3c(c2)oc2c4ccccc4ccc23)cc1]

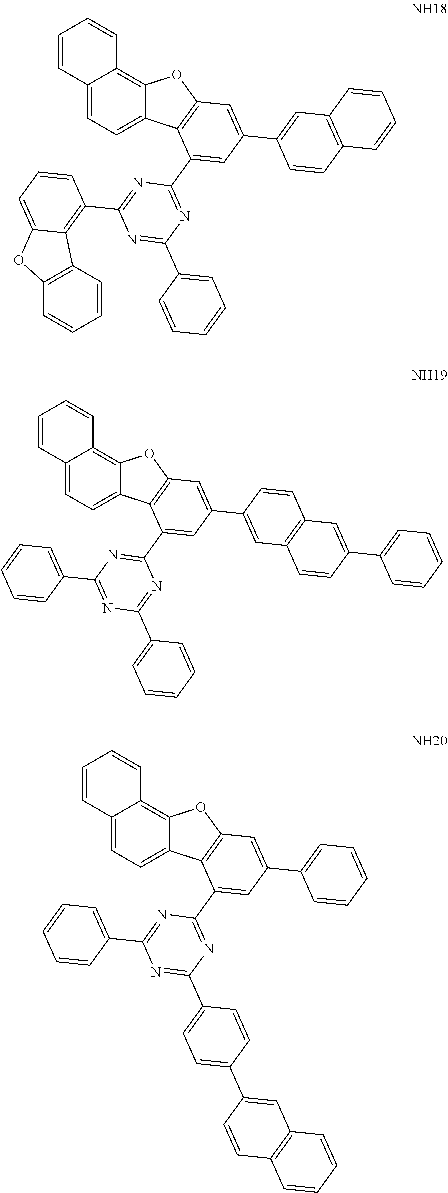 [SMILES: c1ccc(-c2nc(-c3cccc4oc5ccccc5c34)nc(-c3cc(-c4ccc5ccccc5c4)cc4oc5c6ccccc6ccc5c34)n2)cc1 ; c1ccc(-c2ccc3cc(-c4cc(-c5nc(-c6ccccc6)nc(-c6ccccc6)n5)c5c(c4)oc4c6ccccc6ccc45)ccc3c2)cc1 ; c1ccc(-c2cc(-c3nc(-c4ccccc4)nc(-c4ccc(-c5ccc6ccccc6c5)cc4)n3)c3c(c2)oc2c4ccccc4ccc23)cc1]

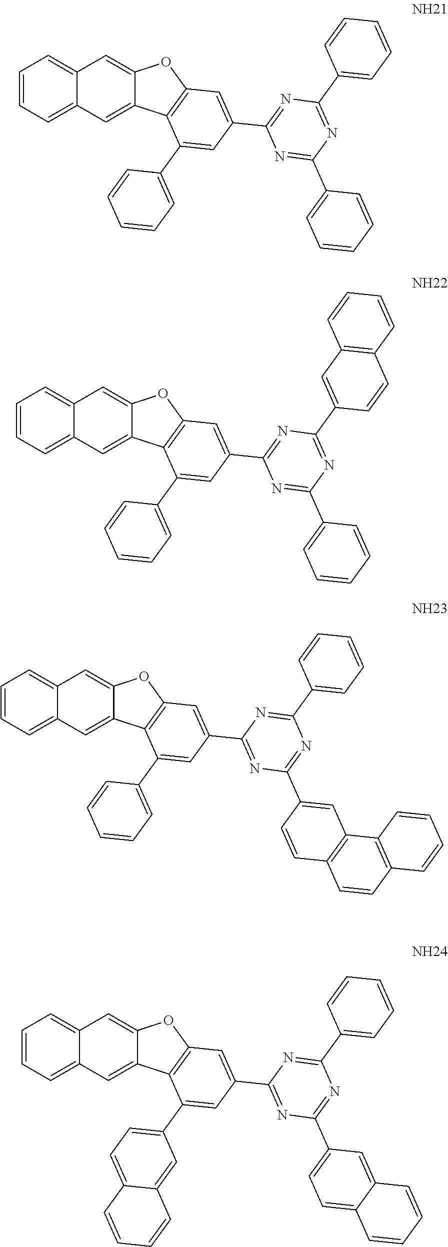

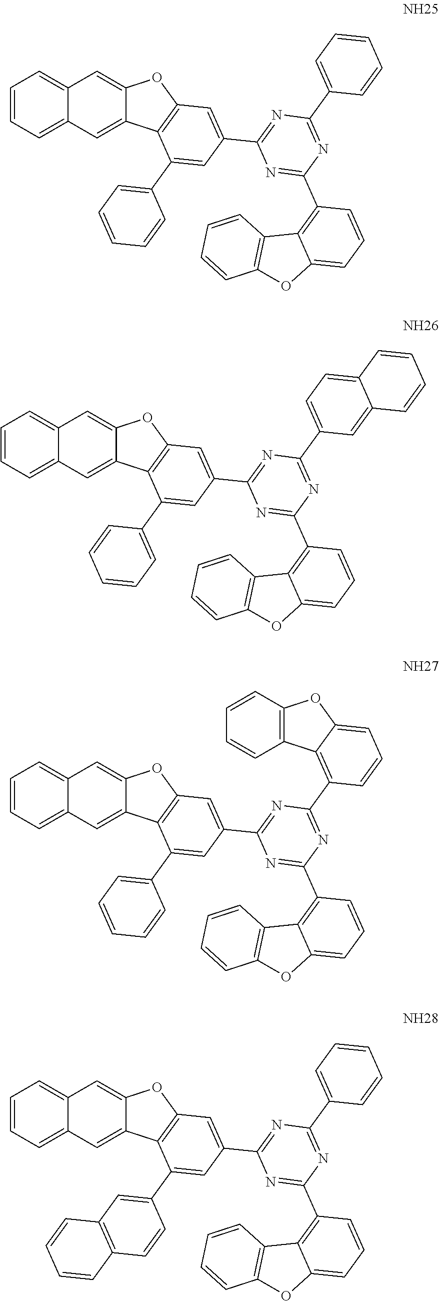 [SMILES: c1ccc(-c2nc(-c3cc(-c4ccccc4)c4c(c3)oc3cc5ccccc5cc34)nc(-c3cccc4oc5ccccc5c34)n2)cc1 ; c1ccc(-c2cc(-c3nc(-c4ccc5ccccc5c4)nc(-c4cccc5oc6ccccc6c45)n3)cc3oc4cc5ccccc5cc4c23)cc1 ; c1ccc(-c2cc(-c3nc(-c4cccc5oc6ccccc6c45)nc(-c4cccc5oc6ccccc6c45)n3)cc3oc4cc5ccccc5cc4c23)cc1 ; c1ccc(-c2nc(-c3cc(-c4ccc5ccccc5c4)c4c(c3)oc3cc5ccccc5cc34)nc(-c3cccc4oc5ccccc5c34)n2)cc1]

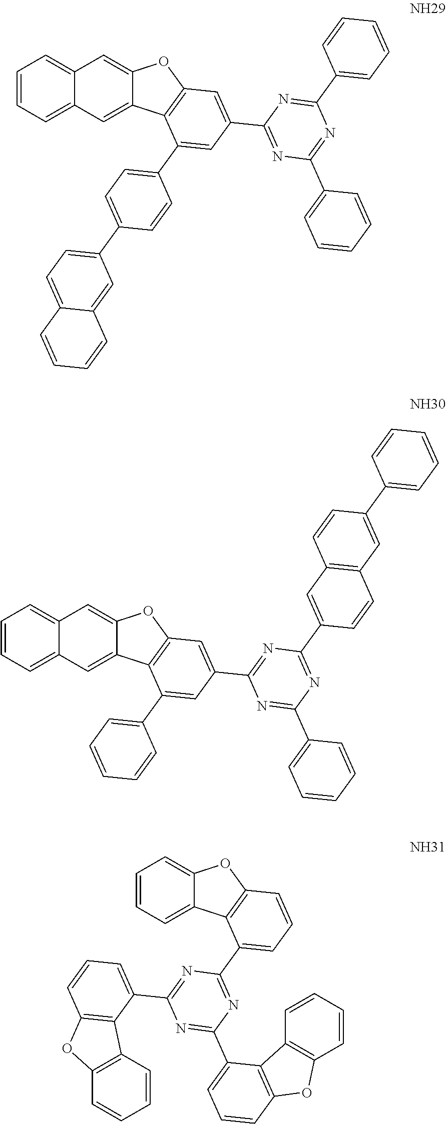 [SMILES: c1ccc(-c2nc(-c3ccccc3)nc(-c3cc(-c4ccc(-c5ccc6ccccc6c5)cc4)c4c(c3)oc3cc5ccccc5cc34)n2)cc1 ; c1ccc(-c2ccc3cc(-c4nc(-c5ccccc5)nc(-c5cc(-c6ccccc6)c6c(c5)oc5cc7ccccc7cc56)n4)ccc3c2)cc1 ; c1ccc2c(c1)oc1cccc(-c3nc(-c4cccc5oc6ccccc6c45)nc(-c4cccc5oc6ccccc6c45)n3)c12]

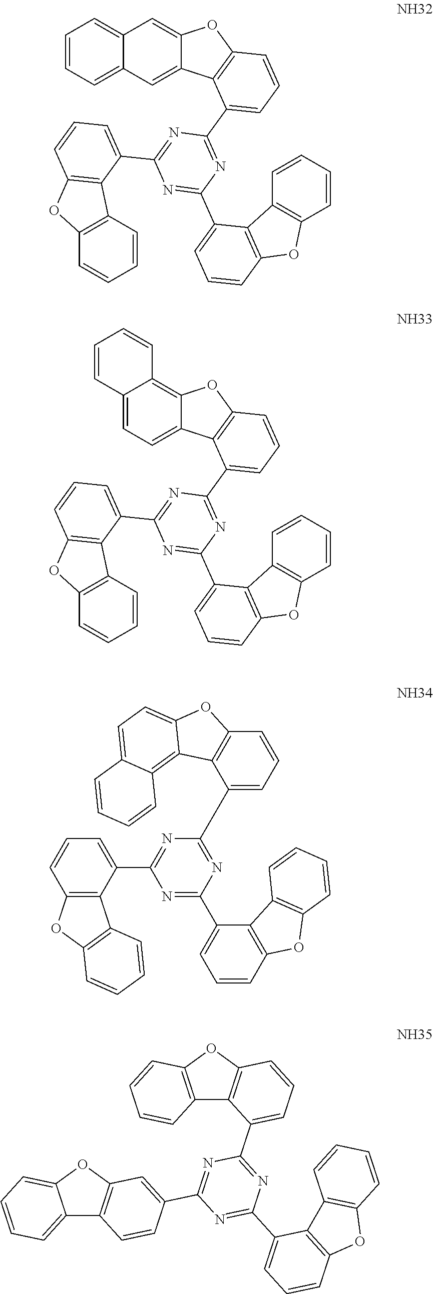 [SMILES: c1ccc2cc3c(cc2c1)oc1cccc(-c2nc(-c4cccc5oc6ccccc6c45)nc(-c4cccc5oc6ccccc6c45)n2)c13 ; c1ccc2c(c1)ccc1c2oc2cccc(-c3nc(-c4cccc5oc6ccccc6c45)nc(-c4cccc5oc6ccccc6c45)n3)c21 ; c1ccc2c(c1)ccc1oc3cccc(-c4nc(-c5cccc6oc7ccccc7c56)nc(-c5cccc6oc7ccccc7c56)n4)c3c12 ; c1ccc2c(c1)oc1cc(-c3nc(-c4cccc5oc6ccccc6c45)nc(-c4cccc5oc6ccccc6c45)n3)ccc12]

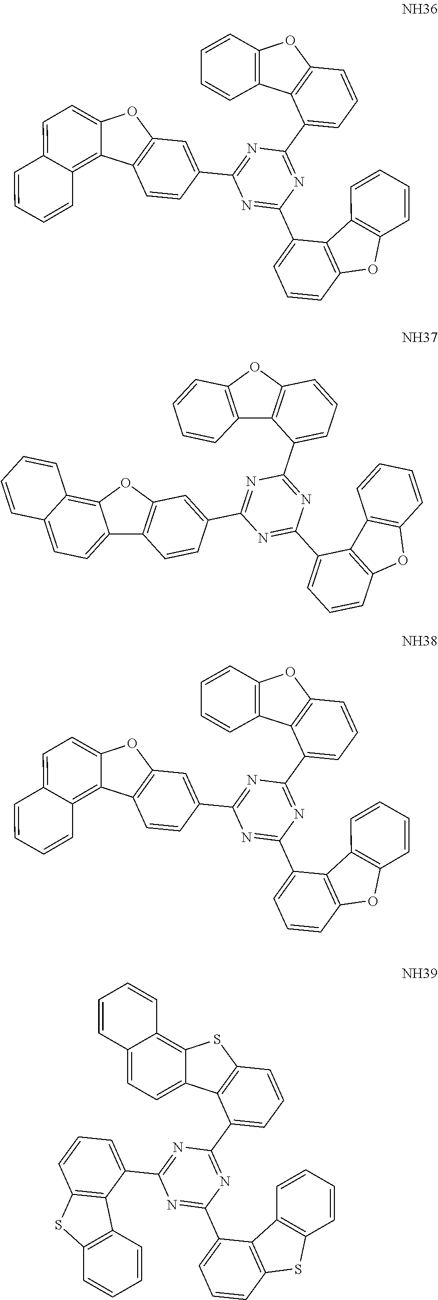 [SMILES: c1ccc2c(c1)ccc1oc3cc(-c4nc(-c5cccc6oc7ccccc7c56)nc(-c5cccc6oc7ccccc7c56)n4)ccc3c12 ; c1ccc2c(c1)ccc1c3ccc(-c4nc(-c5cccc6oc7ccccc7c56)nc(-c5cccc6oc7ccccc7c56)n4)cc3oc21 ; c1ccc2c(c1)ccc1oc3cc(-c4nc(-c5cccc6oc7ccccc7c56)nc(-c5cccc6oc7ccccc7c56)n4)ccc3c12 ; c1ccc2c(c1)ccc1c2sc2cccc(-c3nc(-c4cccc5sc6ccccc6c45)nc(-c4cccc5sc6ccccc6c45)n3)c21]

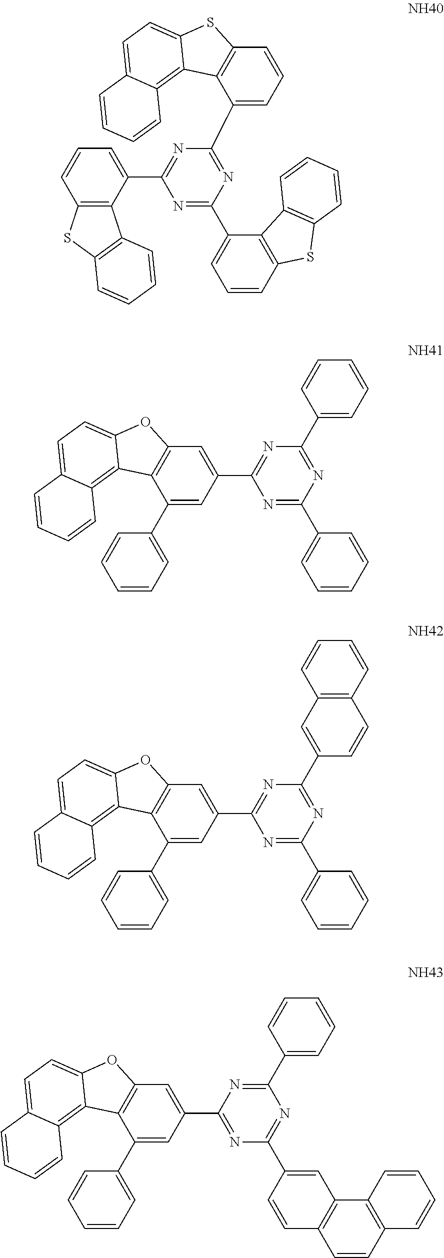 [SMILES: c1ccc2c(c1)ccc1sc3cccc(-c4nc(-c5cccc6sc7ccccc7c56)nc(-c5cccc6sc7ccccc7c56)n4)c3c12 ; c1ccc(-c2nc(-c3ccccc3)nc(-c3cc(-c4ccccc4)c4c(c3)oc3ccc5ccccc5c34)n2)cc1 ; c1ccc(-c2nc(-c3ccc4ccccc4c3)nc(-c3cc(-c4ccccc4)c4c(c3)oc3ccc5ccccc5c34)n2)cc1 ; c1ccc(-c2nc(-c3cc(-c4ccccc4)c4c(c3)oc3ccc5ccccc5c34)nc(-c3ccc4ccc5ccccc5c4c3)n2)cc1]

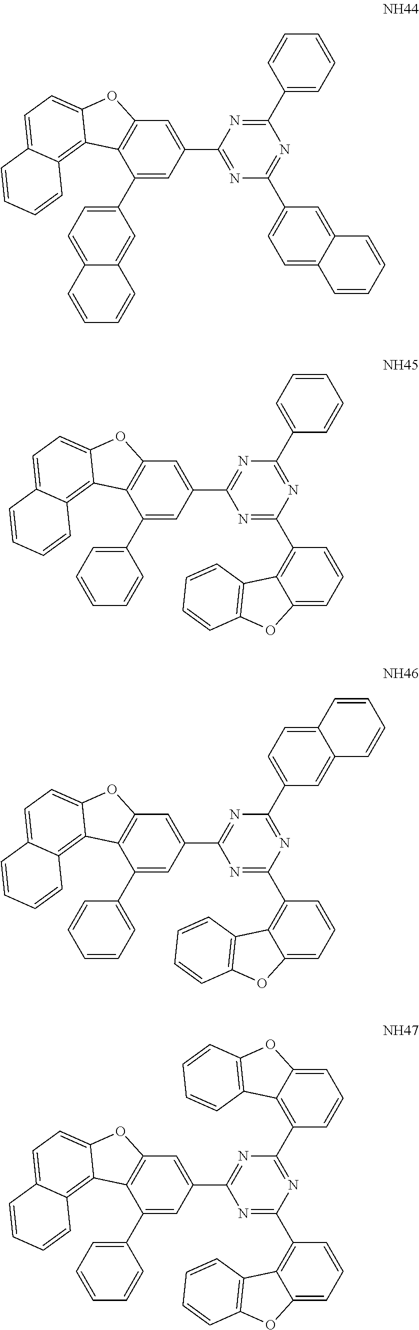 [SMILES: c1ccc(-c2nc(-c3ccc4ccccc4c3)nc(-c3cc(-c4ccc5ccccc5c4)c4c(c3)oc3ccc5ccccc5c34)n2)cc1 ; c1ccc(-c2nc(-c3cc(-c4ccccc4)c4c(c3)oc3ccc5ccccc5c34)nc(-c3cccc4oc5ccccc5c34)n2)cc1 ; c1ccc(-c2cc(-c3nc(-c4ccc5ccccc5c4)nc(-c4cccc5oc6ccccc6c45)n3)cc3oc4ccc5ccccc5c4c23)cc1 ; c1ccc(-c2cc(-c3nc(-c4cccc5oc6ccccc6c45)nc(-c4cccc5oc6ccccc6c45)n3)cc3oc4ccc5ccccc5c4c23)cc1]

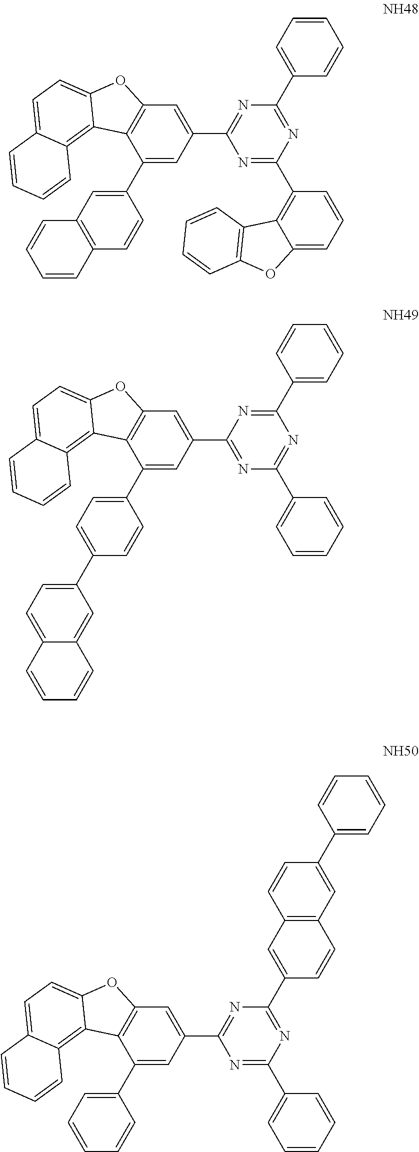 [SMILES: c1ccc(-c2nc(-c3cc(-c4ccc5ccccc5c4)c4c(c3)oc3ccc5ccccc5c34)nc(-c3cccc4oc5ccccc5c34)n2)cc1 ; c1ccc(-c2nc(-c3ccccc3)nc(-c3cc(-c4ccc(-c5ccc6ccccc6c5)cc4)c4c(c3)oc3ccc5ccccc5c34)n2)cc1 ; c1ccc(-c2ccc3cc(-c4nc(-c5ccccc5)nc(-c5cc(-c6ccccc6)c6c(c5)oc5ccc7ccccc7c56)n4)ccc3c2)cc1]

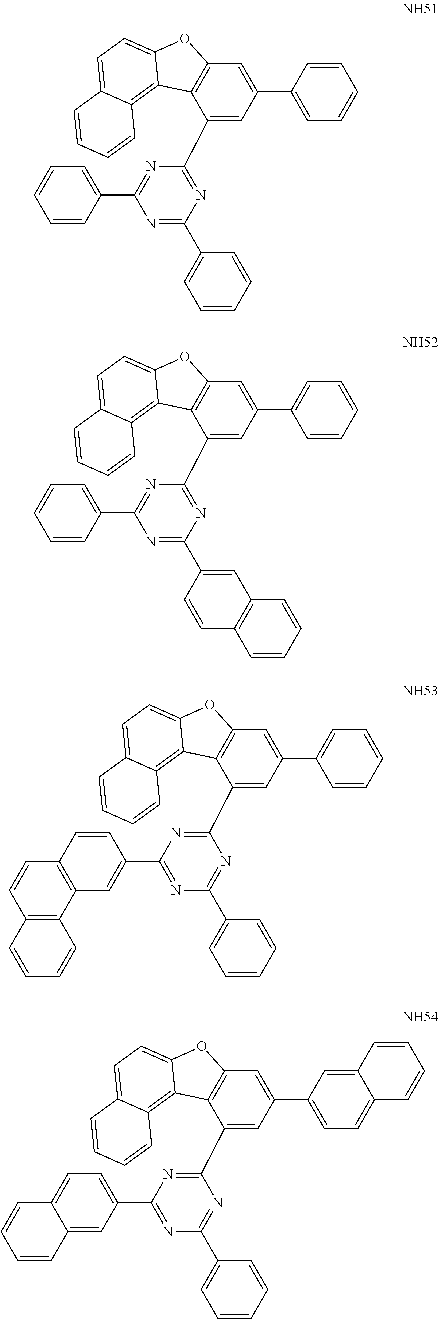 [SMILES: c1ccc(-c2cc(-c3nc(-c4ccccc4)nc(-c4ccccc4)n3)c3c(c2)oc2ccc4ccccc4c23)cc1 ; c1ccc(-c2cc(-c3nc(-c4ccccc4)nc(-c4ccc5ccccc5c4)n3)c3c(c2)oc2ccc4ccccc4c23)cc1 ; c1ccc(-c2cc(-c3nc(-c4ccccc4)nc(-c4ccc5ccc6ccccc6c5c4)n3)c3c(c2)oc2ccc4ccccc4c23)cc1 ; c1ccc(-c2nc(-c3ccc4ccccc4c3)nc(-c3cc(-c4ccc5ccccc5c4)cc4oc5ccc6ccccc6c5c34)n2)cc1]

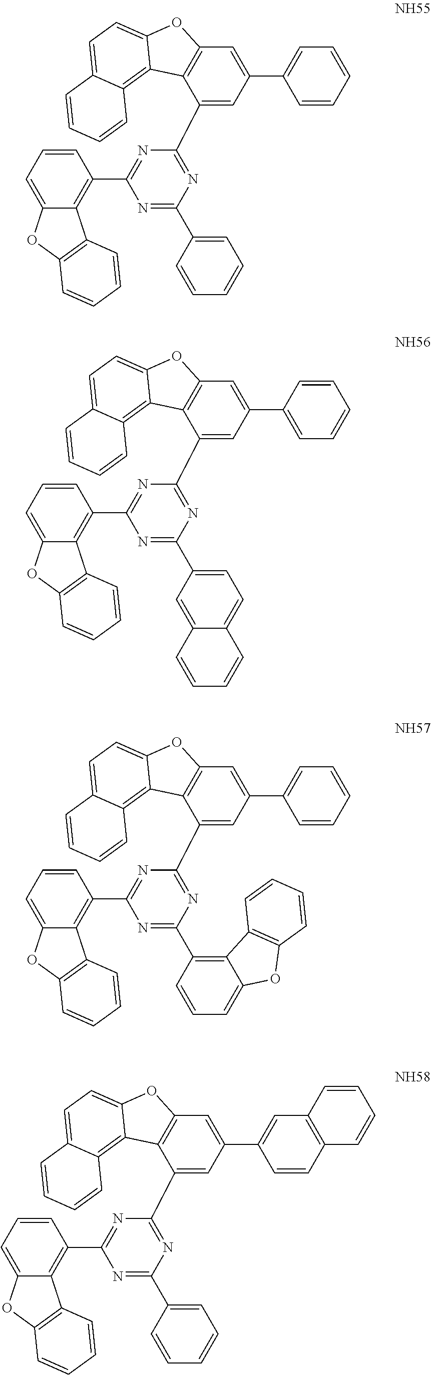 [SMILES: c1ccc(-c2cc(-c3nc(-c4ccccc4)nc(-c4cccc5oc6ccccc6c45)n3)c3c(c2)oc2ccc4ccccc4c23)cc1 ; c1ccc(-c2cc(-c3nc(-c4ccc5ccccc5c4)nc(-c4cccc5oc6ccccc6c45)n3)c3c(c2)oc2ccc4ccccc4c23)cc1 ; c1ccc(-c2cc(-c3nc(-c4cccc5oc6ccccc6c45)nc(-c4cccc5oc6ccccc6c45)n3)c3c(c2)oc2ccc4ccccc4c23)cc1 ; c1ccc(-c2nc(-c3cccc4oc5ccccc5c34)nc(-c3cc(-c4ccc5ccccc5c4)cc4oc5ccc6ccccc6c5c34)n2)cc1]

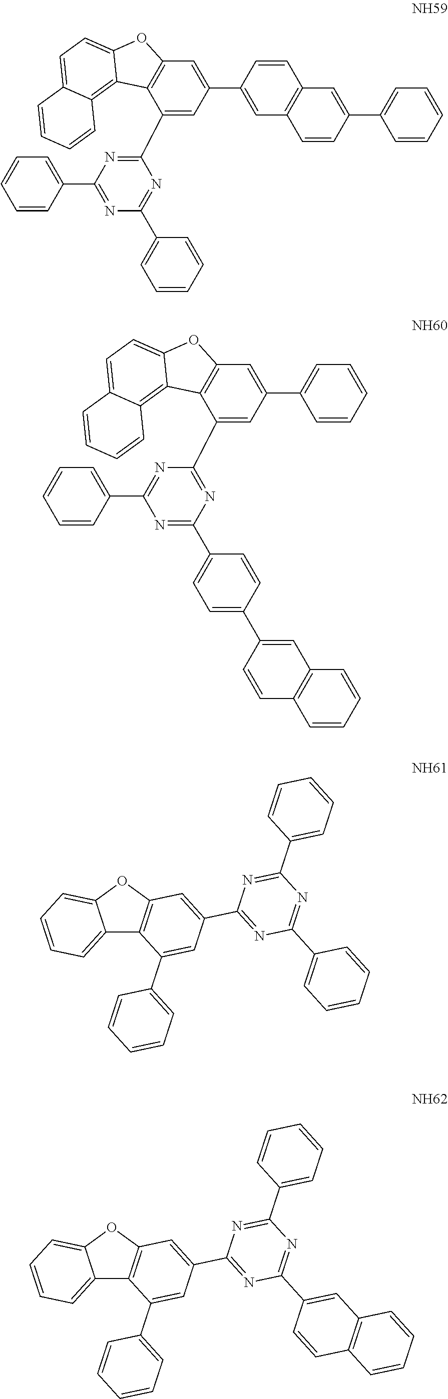 [SMILES: c1ccc(-c2ccc3cc(-c4cc(-c5nc(-c6ccccc6)nc(-c6ccccc6)n5)c5c(c4)oc4ccc6ccccc6c45)ccc3c2)cc1 ; c1ccc(-c2cc(-c3nc(-c4ccccc4)nc(-c4ccc(-c5ccc6ccccc6c5)cc4)n3)c3c(c2)oc2ccc4ccccc4c23)cc1 ; c1ccc(-c2nc(-c3ccccc3)nc(-c3cc(-c4ccccc4)c4c(c3)oc3ccccc34)n2)cc1 ; c1ccc(-c2nc(-c3ccc4ccccc4c3)nc(-c3cc(-c4ccccc4)c4c(c3)oc3ccccc34)n2)cc1]

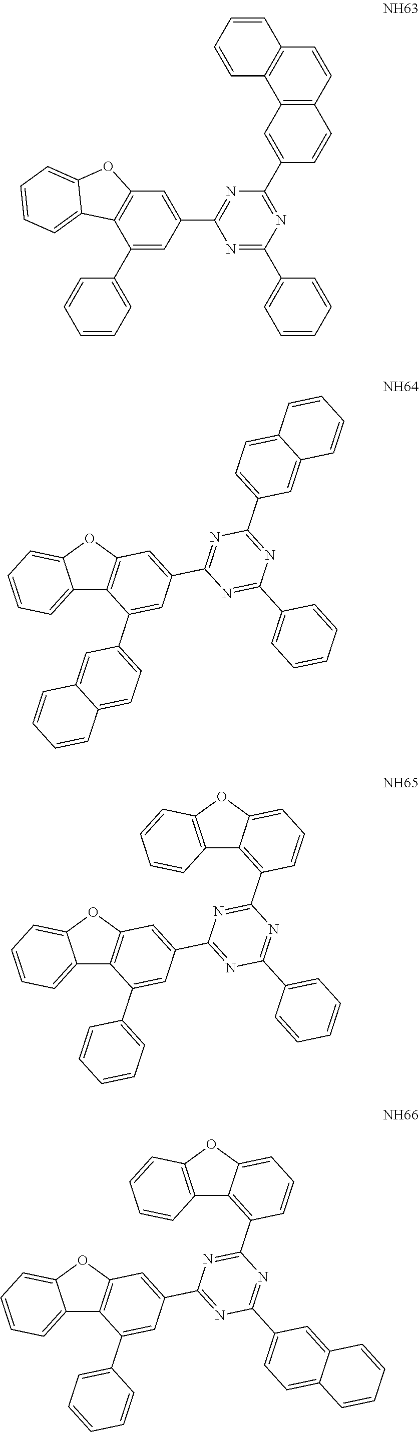 [SMILES: c1ccc(-c2nc(-c3cc(-c4ccccc4)c4c(c3)oc3ccccc34)nc(-c3ccc4ccc5ccccc5c4c3)n2)cc1 ; c1ccc(-c2nc(-c3ccc4ccccc4c3)nc(-c3cc(-c4ccc5ccccc5c4)c4c(c3)oc3ccccc34)n2)cc1 ; c1ccc(-c2nc(-c3cc(-c4ccccc4)c4c(c3)oc3ccccc34)nc(-c3cccc4oc5ccccc5c34)n2)cc1 ; c1ccc(-c2cc(-c3nc(-c4ccc5ccccc5c4)nc(-c4cccc5oc6ccccc6c45)n3)cc3oc4ccccc4c23)cc1]

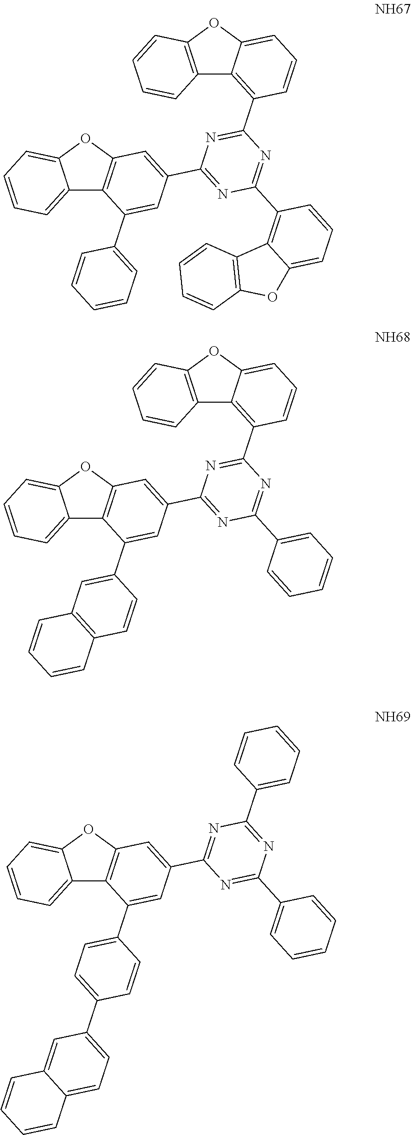

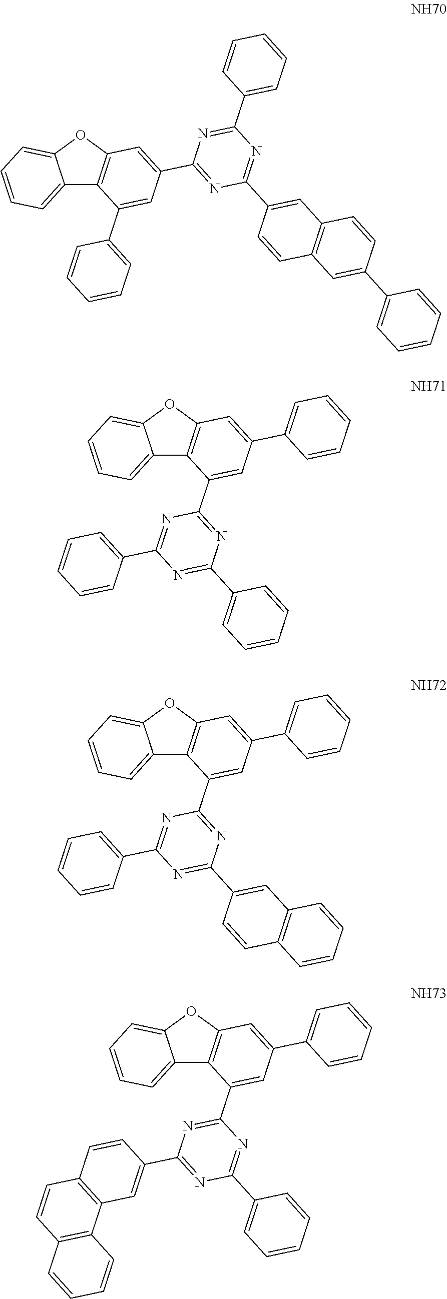

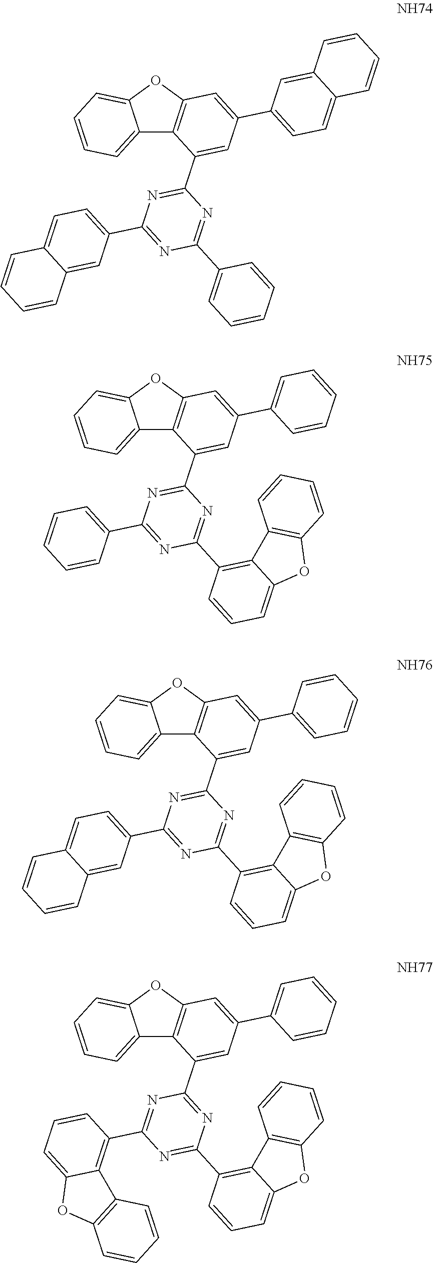 [SMILES: c1ccc(-c2nc(-c3ccc4ccccc4c3)nc(-c3cc(-c4ccc5ccccc5c4)cc4oc5ccccc5c34)n2)cc1 ; c1ccc(-c2cc(-c3nc(-c4ccccc4)nc(-c4cccc5oc6ccccc6c45)n3)c3c(c2)oc2ccccc23)cc1 ; c1ccc(-c2cc(-c3nc(-c4ccc5ccccc5c4)nc(-c4cccc5oc6ccccc6c45)n3)c3c(c2)oc2ccccc23)cc1 ; c1ccc(-c2cc(-c3nc(-c4cccc5oc6ccccc6c45)nc(-c4cccc5oc6ccccc6c45)n3)c3c(c2)oc2ccccc23)cc1]

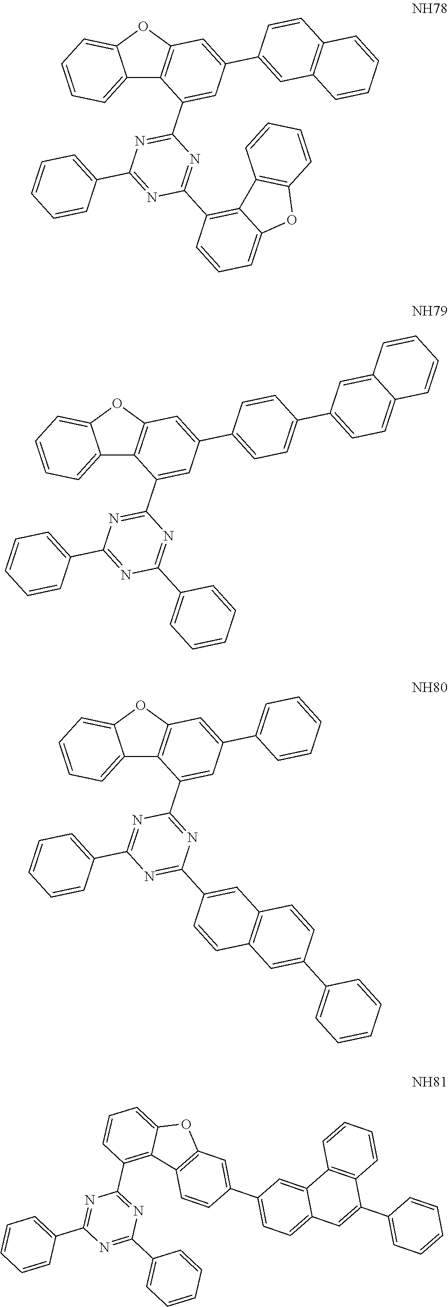 [SMILES: c1ccc(-c2nc(-c3cccc4oc5ccccc5c34)nc(-c3cc(-c4ccc5ccccc5c4)cc4oc5ccccc5c34)n2)cc1 ; c1ccc(-c2nc(-c3ccccc3)nc(-c3cc(-c4ccc(-c5ccc6ccccc6c5)cc4)cc4oc5ccccc5c34)n2)cc1 ; c1ccc(-c2ccc3cc(-c4nc(-c5ccccc5)nc(-c5cc(-c6ccccc6)cc6oc7ccccc7c56)n4)ccc3c2)cc1 ; c1ccc(-c2nc(-c3ccccc3)nc(-c3cccc4oc5cc(-c6ccc7cc(-c8ccccc8)c8ccccc8c7c6)ccc5c34)n2)cc1]

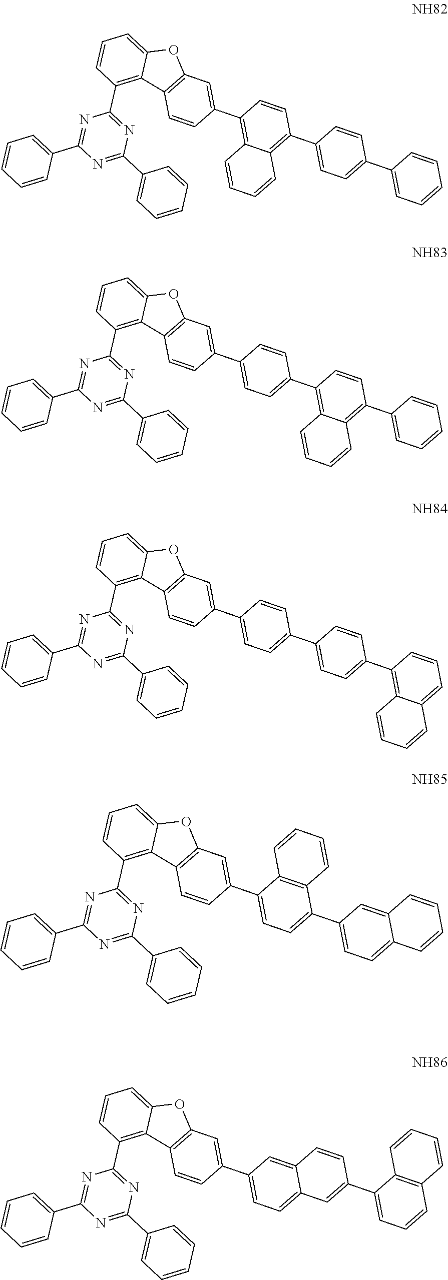 [SMILES: c1ccc(-c2ccc(-c3ccc(-c4ccc5c(c4)oc4cccc(-c6nc(-c7ccccc7)nc(-c7ccccc7)n6)c45)c4ccccc34)cc2)cc1 ; c1ccc(-c2nc(-c3ccccc3)nc(-c3cccc4oc5cc(-c6ccc(-c7ccc(-c8ccccc8)c8ccccc78)cc6)ccc5c34)n2)cc1 ; c1ccc(-c2nc(-c3ccccc3)nc(-c3cccc4oc5cc(-c6ccc(-c7ccc(-c8cccc9ccccc89)cc7)cc6)ccc5c34)n2)cc1 ; c1ccc(-c2nc(-c3ccccc3)nc(-c3cccc4oc5cc(-c6ccc(-c7ccc8ccccc8c7)c7ccccc67)ccc5c34)n2)cc1 ; c1ccc(-c2nc(-c3ccccc3)nc(-c3cccc4oc5cc(-c6ccc7cc(-c8cccc9ccccc89)ccc7c6)ccc5c34)n2)cc1]

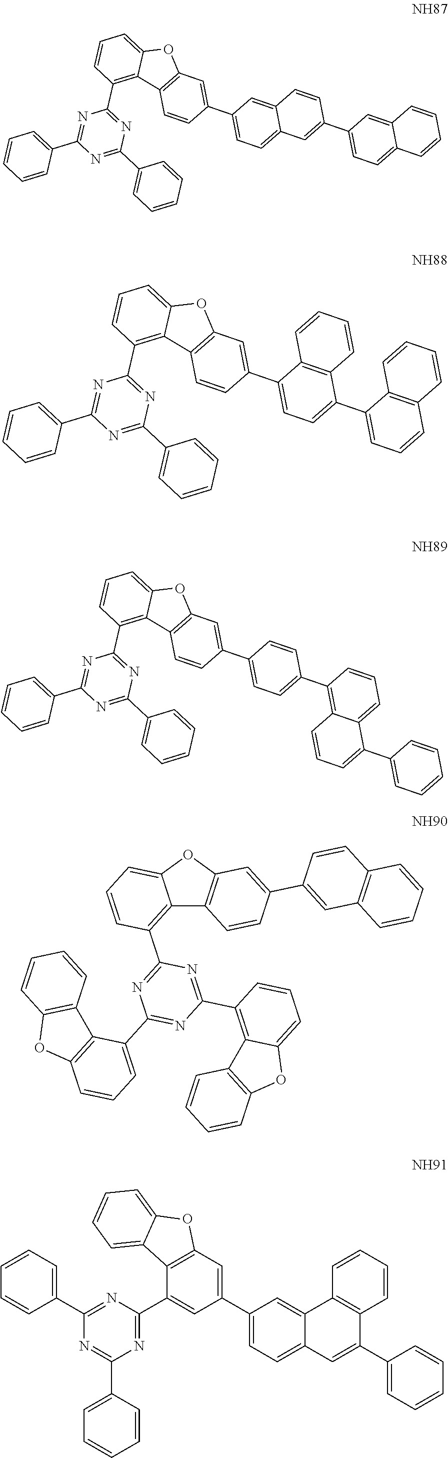 [SMILES: c1ccc(-c2nc(-c3ccccc3)nc(-c3cccc4oc5cc(-c6ccc7cc(-c8ccc9ccccc9c8)ccc7c6)ccc5c34)n2)cc1 ; c1ccc(-c2nc(-c3ccccc3)nc(-c3cccc4oc5cc(-c6ccc(-c7cccc8ccccc78)c7ccccc67)ccc5c34)n2)cc1 ; c1ccc(-c2nc(-c3ccccc3)nc(-c3cccc4oc5cc(-c6ccc(-c7cccc8c(-c9ccccc9)cccc78)cc6)ccc5c34)n2)cc1 ; c1ccc2cc(-c3ccc4c(c3)oc3cccc(-c5nc(-c6cccc7oc8ccccc8c67)nc(-c6cccc7oc8ccccc8c67)n5)c34)ccc2c1 ; c1ccc(-c2nc(-c3ccccc3)nc(-c3cc(-c4ccc5cc(-c6ccccc6)c6ccccc6c5c4)cc4oc5ccccc5c34)n2)cc1]

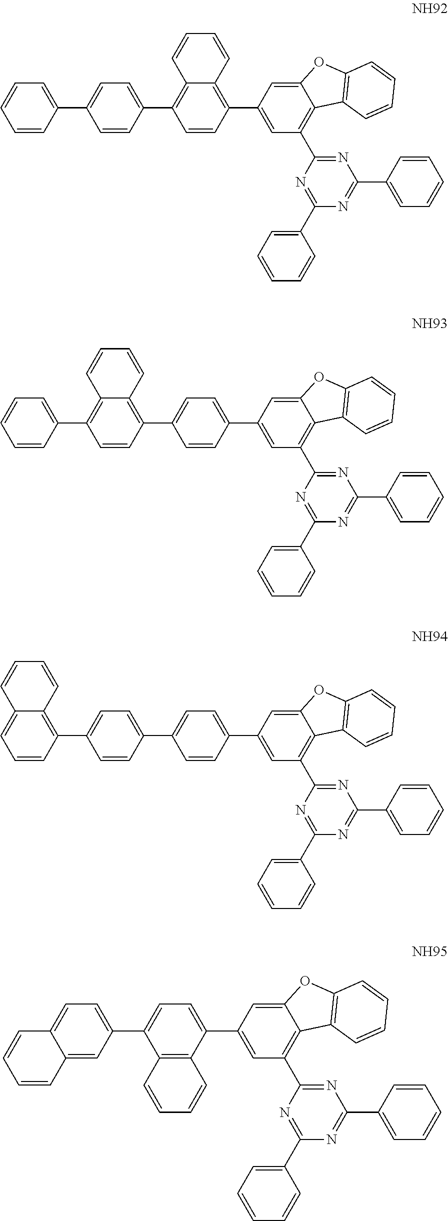 [SMILES: c1ccc(-c2ccc(-c3ccc(-c4cc(-c5nc(-c6ccccc6)nc(-c6ccccc6)n5)c5c(c4)oc4ccccc45)c4ccccc34)cc2)cc1 ; c1ccc(-c2nc(-c3ccccc3)nc(-c3cc(-c4ccc(-c5ccc(-c6ccccc6)c6ccccc56)cc4)cc4oc5ccccc5c34)n2)cc1 ; c1ccc(-c2nc(-c3ccccc3)nc(-c3cc(-c4ccc(-c5ccc(-c6cccc7ccccc67)cc5)cc4)cc4oc5ccccc5c34)n2)cc1 ; c1ccc(-c2nc(-c3ccccc3)nc(-c3cc(-c4ccc(-c5ccc6ccccc6c5)c5ccccc45)cc4oc5ccccc5c34)n2)cc1]

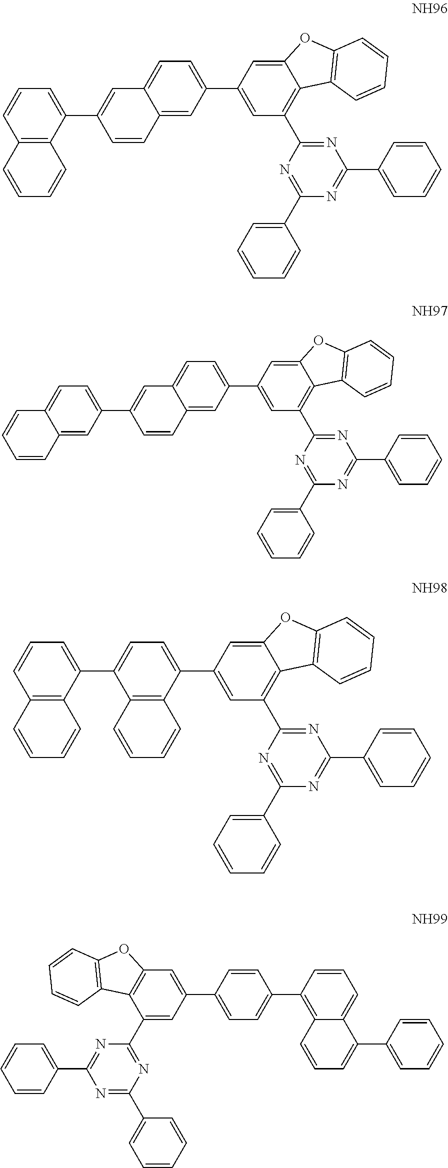 [SMILES: c1ccc(-c2nc(-c3ccccc3)nc(-c3cc(-c4ccc5cc(-c6cccc7ccccc67)ccc5c4)cc4oc5ccccc5c34)n2)cc1 ; c1ccc(-c2nc(-c3ccccc3)nc(-c3cc(-c4ccc5cc(-c6ccc7ccccc7c6)ccc5c4)cc4oc5ccccc5c34)n2)cc1 ; c1ccc(-c2nc(-c3ccccc3)nc(-c3cc(-c4ccc(-c5cccc6ccccc56)c5ccccc45)cc4oc5ccccc5c34)n2)cc1 ; c1ccc(-c2nc(-c3ccccc3)nc(-c3cc(-c4ccc(-c5cccc6c(-c7ccccc7)cccc56)cc4)cc4oc5ccccc5c34)n2)cc1]

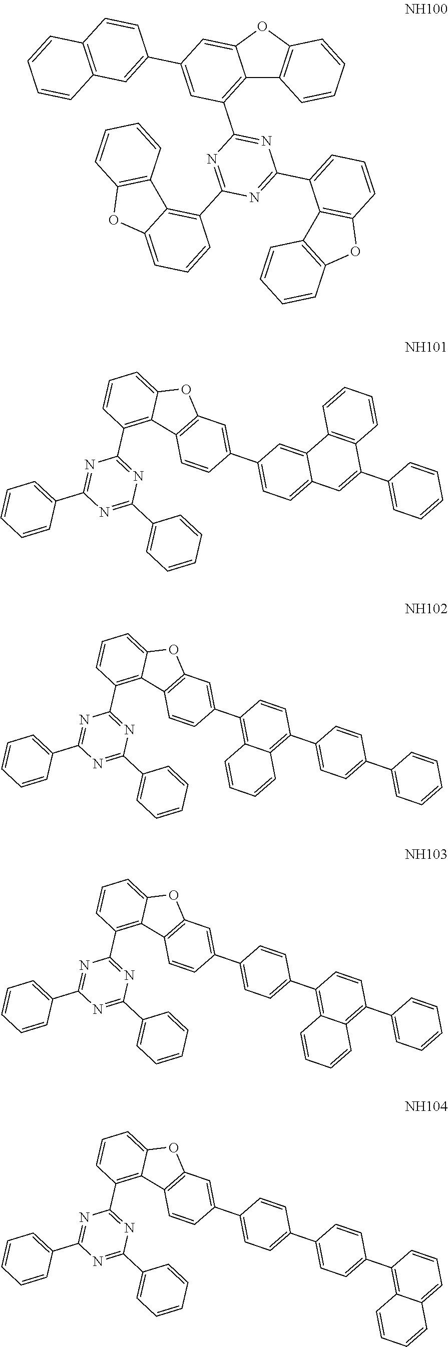 [SMILES: c1ccc2cc(-c3cc(-c4nc(-c5cccc6oc7ccccc7c56)nc(-c5cccc6oc7ccccc7c56)n4)c4c(c3)oc3ccccc34)ccc2c1 ; c1ccc(-c2nc(-c3ccccc3)nc(-c3cccc4oc5cc(-c6ccc7cc(-c8ccccc8)c8ccccc8c7c6)ccc5c34)n2)cc1 ; c1ccc(-c2ccc(-c3ccc(-c4ccc5c(c4)oc4cccc(-c6nc(-c7ccccc7)nc(-c7ccccc7)n6)c45)c4ccccc34)cc2)cc1 ; c1ccc(-c2nc(-c3ccccc3)nc(-c3cccc4oc5cc(-c6ccc(-c7ccc(-c8ccccc8)c8ccccc78)cc6)ccc5c34)n2)cc1 ; c1ccc(-c2nc(-c3ccccc3)nc(-c3cccc4oc5cc(-c6ccc(-c7ccc(-c8cccc9ccccc89)cc7)cc6)ccc5c34)n2)cc1]

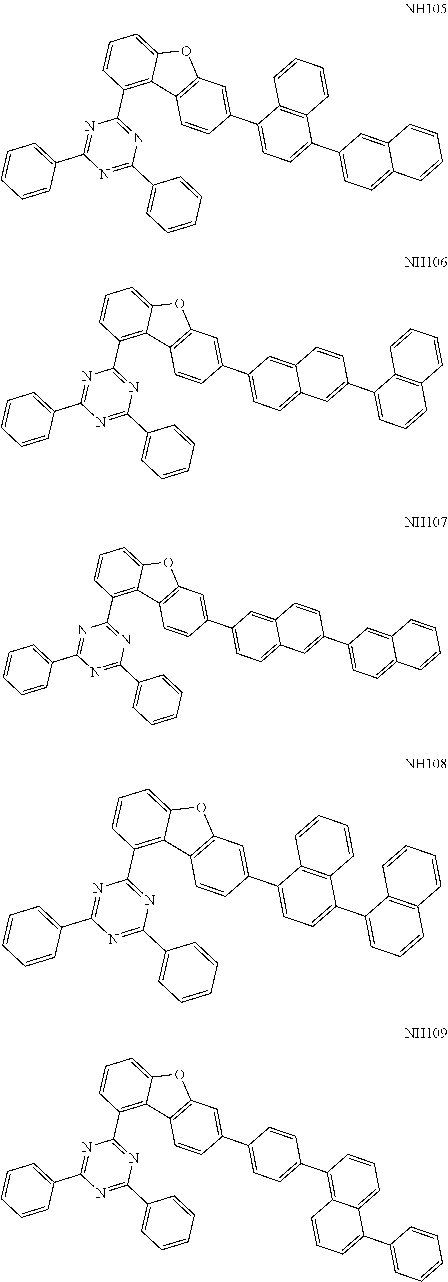 [SMILES: c1ccc(-c2nc(-c3ccccc3)nc(-c3cccc4oc5cc(-c6ccc(-c7ccc8ccccc8c7)c7ccccc67)ccc5c34)n2)cc1 ; c1ccc(-c2nc(-c3ccccc3)nc(-c3cccc4oc5cc(-c6ccc7cc(-c8cccc9ccccc89)ccc7c6)ccc5c34)n2)cc1 ; c1ccc(-c2nc(-c3ccccc3)nc(-c3cccc4oc5cc(-c6ccc7cc(-c8ccc9ccccc9c8)ccc7c6)ccc5c34)n2)cc1 ; c1ccc(-c2nc(-c3ccccc3)nc(-c3cccc4oc5cc(-c6ccc(-c7cccc8ccccc78)c7ccccc67)ccc5c34)n2)cc1 ; c1ccc(-c2nc(-c3ccccc3)nc(-c3cccc4oc5cc(-c6ccc(-c7cccc8c(-c9ccccc9)cccc78)cc6)ccc5c34)n2)cc1]

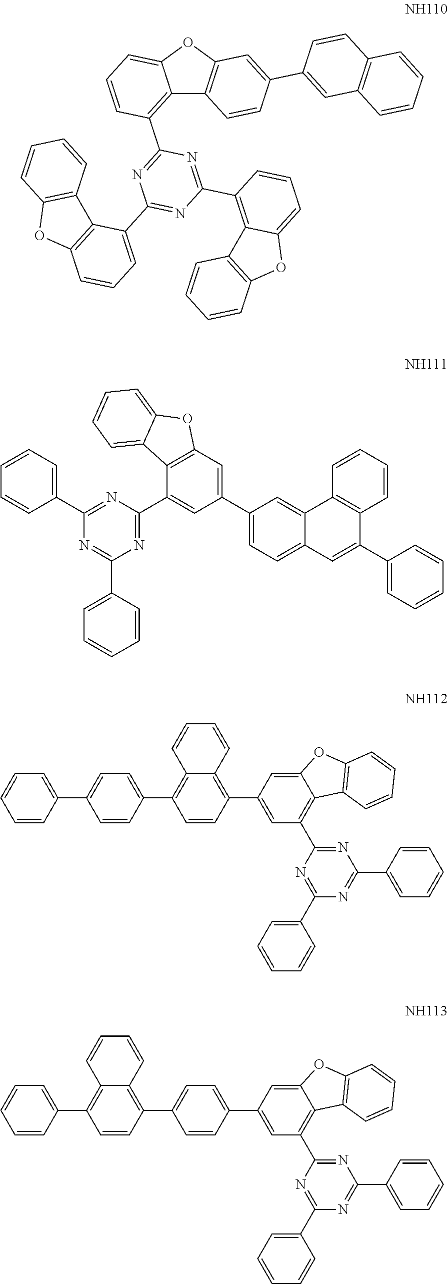 [SMILES: c1ccc2cc(-c3ccc4c(c3)oc3cccc(-c5nc(-c6cccc7oc8ccccc8c67)nc(-c6cccc7oc8ccccc8c67)n5)c34)ccc2c1 ; c1ccc(-c2nc(-c3ccccc3)nc(-c3cc(-c4ccc5cc(-c6ccccc6)c6ccccc6c5c4)cc4oc5ccccc5c34)n2)cc1 ; c1ccc(-c2ccc(-c3ccc(-c4cc(-c5nc(-c6ccccc6)nc(-c6ccccc6)n5)c5c(c4)oc4ccccc45)c4ccccc34)cc2)cc1 ; c1ccc(-c2nc(-c3ccccc3)nc(-c3cc(-c4ccc(-c5ccc(-c6ccccc6)c6ccccc56)cc4)cc4oc5ccccc5c34)n2)cc1]

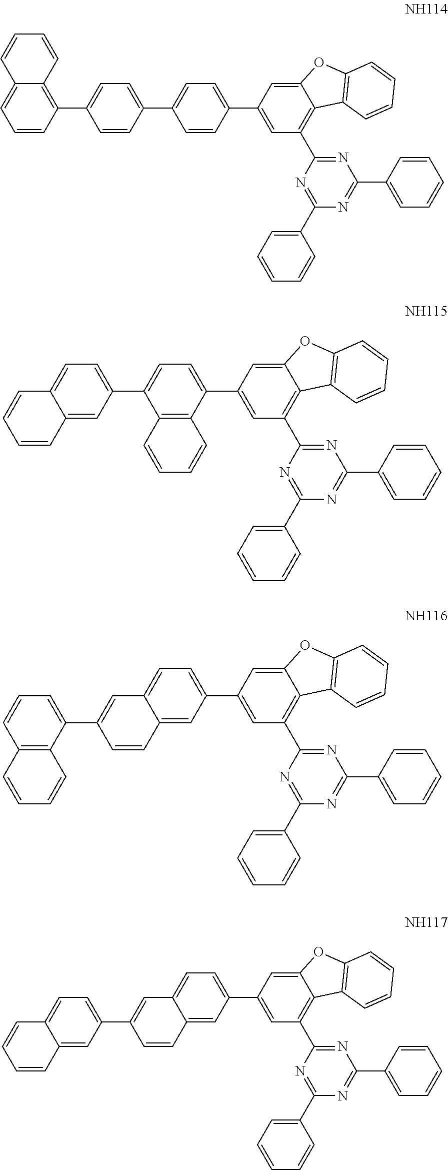 [SMILES: c1ccc(-c2nc(-c3ccccc3)nc(-c3cc(-c4ccc(-c5ccc(-c6cccc7ccccc67)cc5)cc4)cc4oc5ccccc5c34)n2)cc1 ; c1ccc(-c2nc(-c3ccccc3)nc(-c3cc(-c4ccc(-c5ccc6ccccc6c5)c5ccccc45)cc4oc5ccccc5c34)n2)cc1 ; c1ccc(-c2nc(-c3ccccc3)nc(-c3cc(-c4ccc5cc(-c6cccc7ccccc67)ccc5c4)cc4oc5ccccc5c34)n2)cc1 ; c1ccc(-c2nc(-c3ccccc3)nc(-c3cc(-c4ccc5cc(-c6ccc7ccccc7c6)ccc5c4)cc4oc5ccccc5c34)n2)cc1]

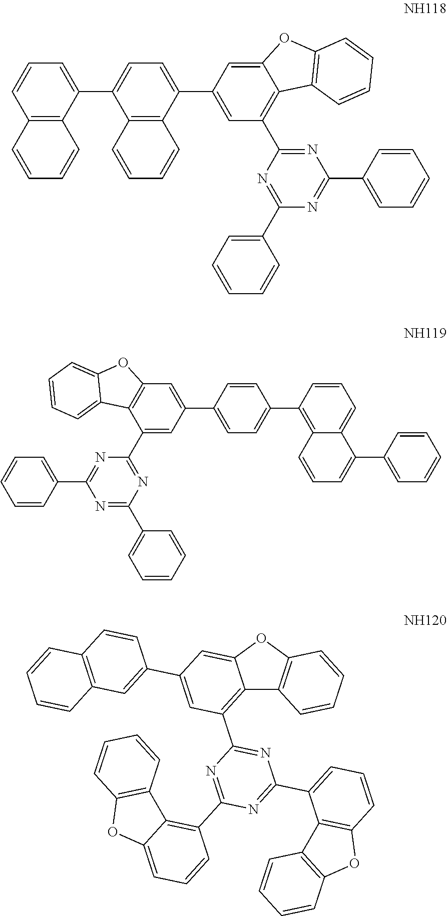 [SMILES: c1ccc(-c2nc(-c3ccccc3)nc(-c3cc(-c4ccc(-c5cccc6ccccc56)c5ccccc45)cc4oc5ccccc5c34)n2)cc1 ; c1ccc(-c2nc(-c3ccccc3)nc(-c3cc(-c4ccc(-c5cccc6c(-c7ccccc7)cccc56)cc4)cc4oc5ccccc5c34)n2)cc1 ; c1ccc2cc(-c3cc(-c4nc(-c5cccc6oc7ccccc7c56)nc(-c5cccc6oc7ccccc7c56)n4)c4c(c3)oc3ccccc34)ccc2c1]

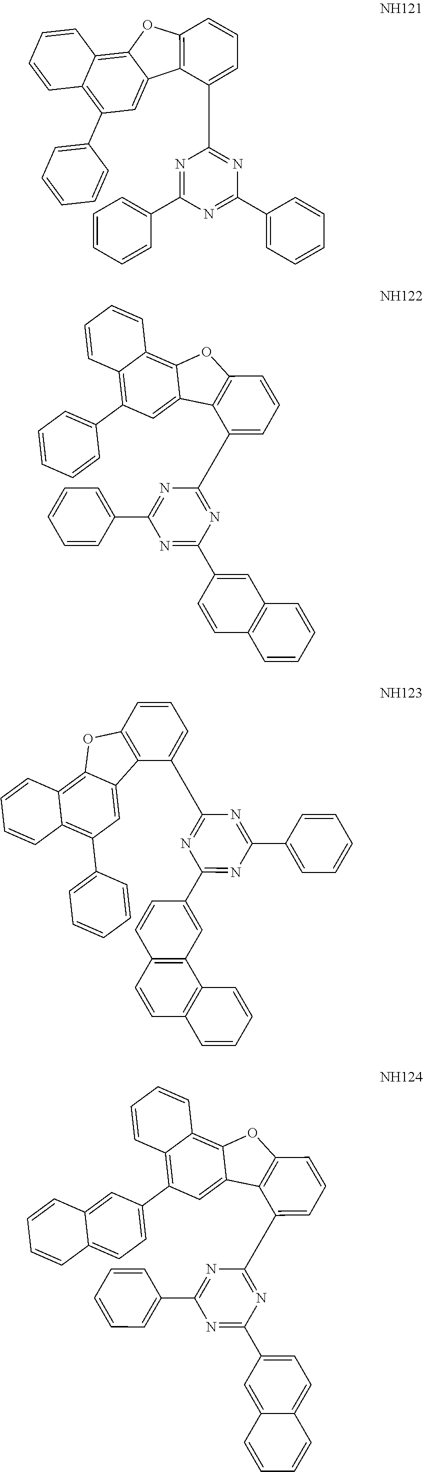

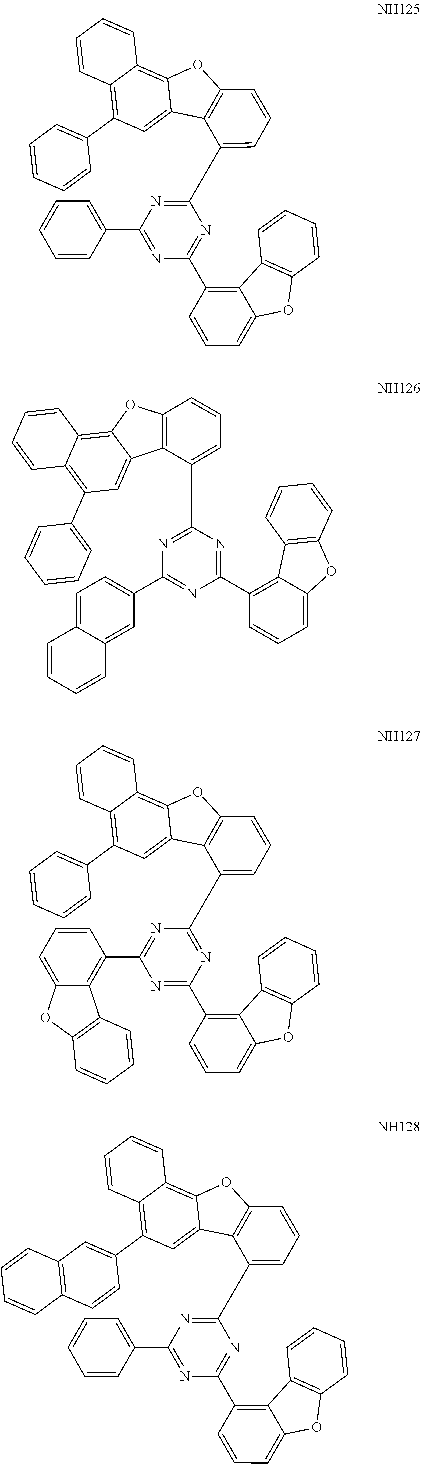 [SMILES: c1ccc(-c2nc(-c3cccc4oc5ccccc5c34)nc(-c3cccc4oc5c6ccccc6c(-c6ccccc6)cc5c34)n2)cc1 ; c1ccc(-c2cc3c(oc4cccc(-c5nc(-c6ccc7ccccc7c6)nc(-c6cccc7oc8ccccc8c67)n5)c43)c3ccccc23)cc1 ; c1ccc(-c2cc3c(oc4cccc(-c5nc(-c6cccc7oc8ccccc8c67)nc(-c6cccc7oc8ccccc8c67)n5)c43)c3ccccc23)cc1 ; c1ccc(-c2nc(-c3cccc4oc5ccccc5c34)nc(-c3cccc4oc5c6ccccc6c(-c6ccc7ccccc7c6)cc5c34)n2)cc1]

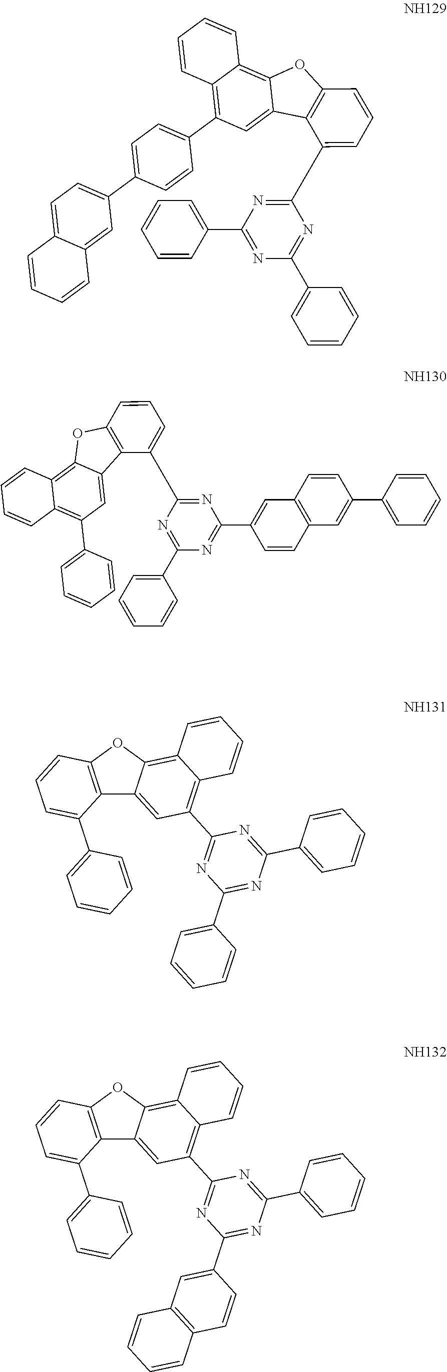 [SMILES: c1ccc(-c2nc(-c3ccccc3)nc(-c3cccc4oc5c6ccccc6c(-c6ccc(-c7ccc8ccccc8c7)cc6)cc5c34)n2)cc1 ; c1ccc(-c2ccc3cc(-c4nc(-c5ccccc5)nc(-c5cccc6oc7c8ccccc8c(-c8ccccc8)cc7c56)n4)ccc3c2)cc1 ; c1ccc(-c2nc(-c3ccccc3)nc(-c3cc4c(oc5cccc(-c6ccccc6)c54)c4ccccc34)n2)cc1 ; c1ccc(-c2nc(-c3ccc4ccccc4c3)nc(-c3cc4c(oc5cccc(-c6ccccc6)c54)c4ccccc34)n2)cc1]

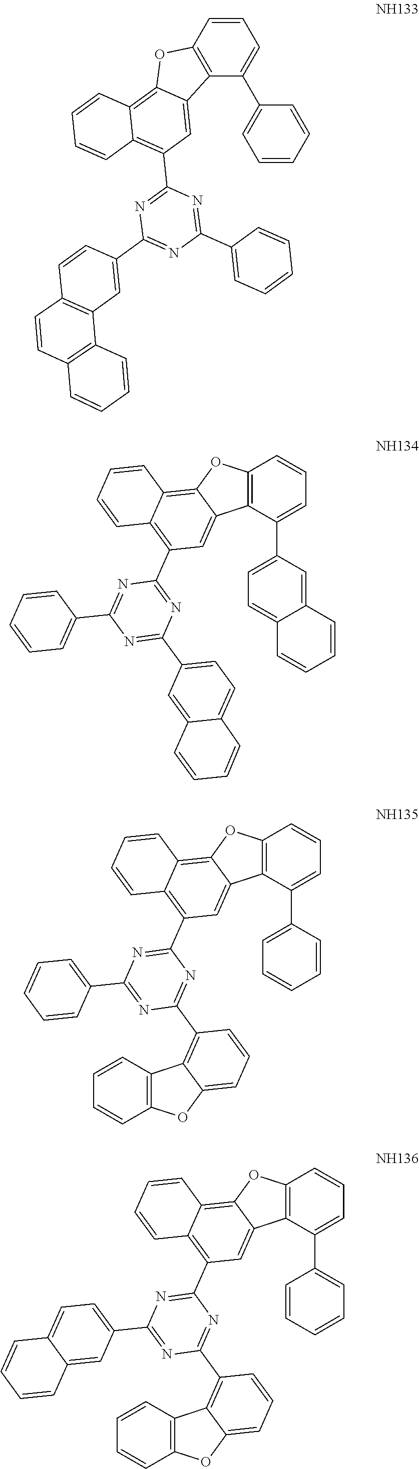 [SMILES: c1ccc(-c2nc(-c3ccc4ccc5ccccc5c4c3)nc(-c3cc4c(oc5cccc(-c6ccccc6)c54)c4ccccc34)n2)cc1 ; c1ccc(-c2nc(-c3ccc4ccccc4c3)nc(-c3cc4c(oc5cccc(-c6ccc7ccccc7c6)c54)c4ccccc34)n2)cc1 ; c1ccc(-c2nc(-c3cc4c(oc5cccc(-c6ccccc6)c54)c4ccccc34)nc(-c3cccc4oc5ccccc5c34)n2)cc1 ; c1ccc(-c2cccc3oc4c5ccccc5c(-c5nc(-c6ccc7ccccc7c6)nc(-c6cccc7oc8ccccc8c67)n5)cc4c23)cc1]

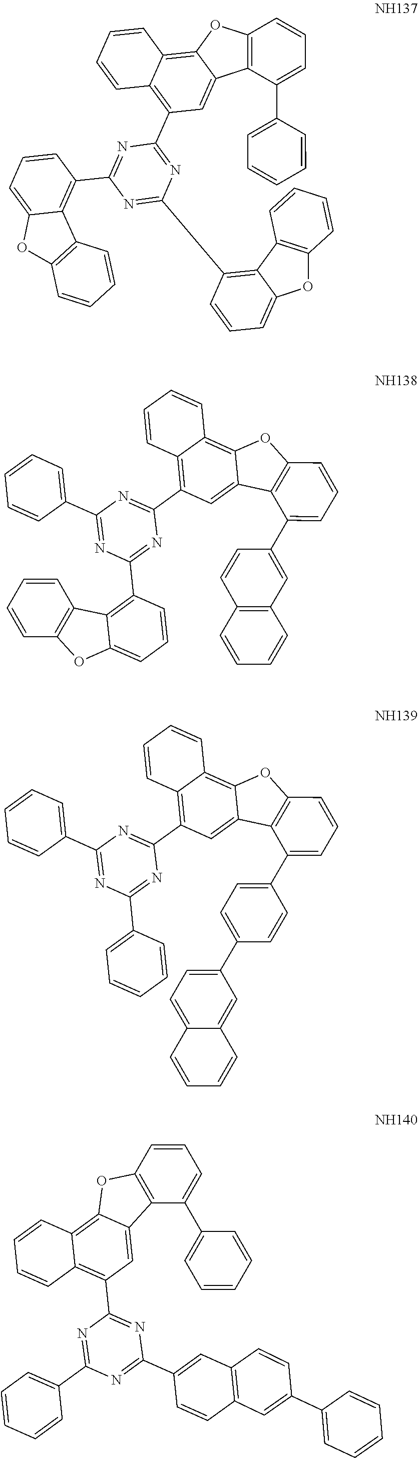 [SMILES: c1ccc(-c2cccc3oc4c5ccccc5c(-c5nc(-c6cccc7oc8ccccc8c67)nc(-c6cccc7oc8ccccc8c67)n5)cc4c23)cc1 ; c1ccc(-c2nc(-c3cc4c(oc5cccc(-c6ccc7ccccc7c6)c54)c4ccccc34)nc(-c3cccc4oc5ccccc5c34)n2)cc1 ; c1ccc(-c2nc(-c3ccccc3)nc(-c3cc4c(oc5cccc(-c6ccc(-c7ccc8ccccc8c7)cc6)c54)c4ccccc34)n2)cc1 ; c1ccc(-c2ccc3cc(-c4nc(-c5ccccc5)nc(-c5cc6c(oc7cccc(-c8ccccc8)c76)c6ccccc56)n4)ccc3c2)cc1]

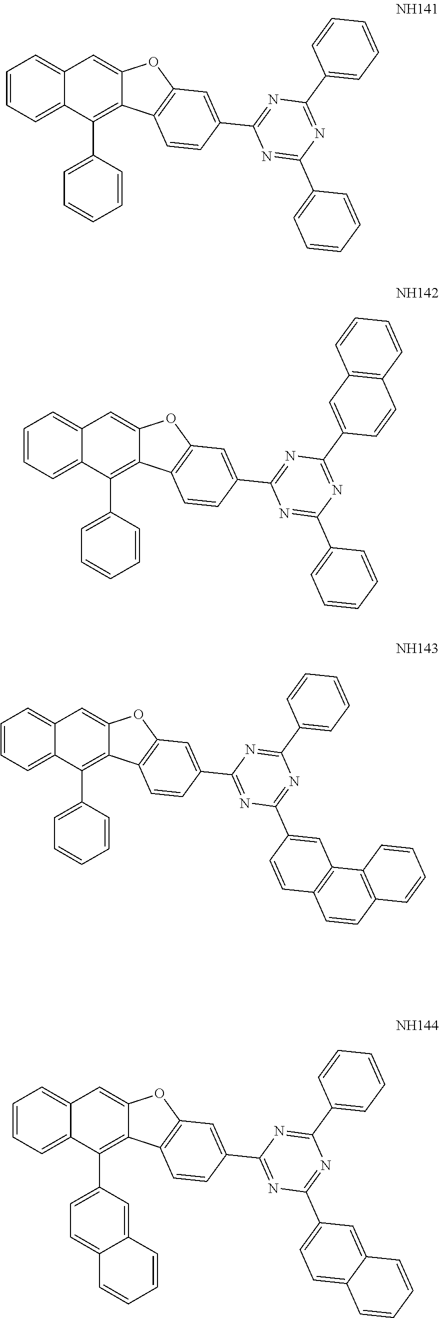 [SMILES: c1ccc(-c2nc(-c3ccccc3)nc(-c3ccc4c(c3)oc3cc5ccccc5c(-c5ccccc5)c34)n2)cc1 ; c1ccc(-c2nc(-c3ccc4ccccc4c3)nc(-c3ccc4c(c3)oc3cc5ccccc5c(-c5ccccc5)c34)n2)cc1 ; c1ccc(-c2nc(-c3ccc4c(c3)oc3cc5ccccc5c(-c5ccccc5)c34)nc(-c3ccc4ccc5ccccc5c4c3)n2)cc1 ; c1ccc(-c2nc(-c3ccc4ccccc4c3)nc(-c3ccc4c(c3)oc3cc5ccccc5c(-c5ccc6ccccc6c5)c34)n2)cc1]

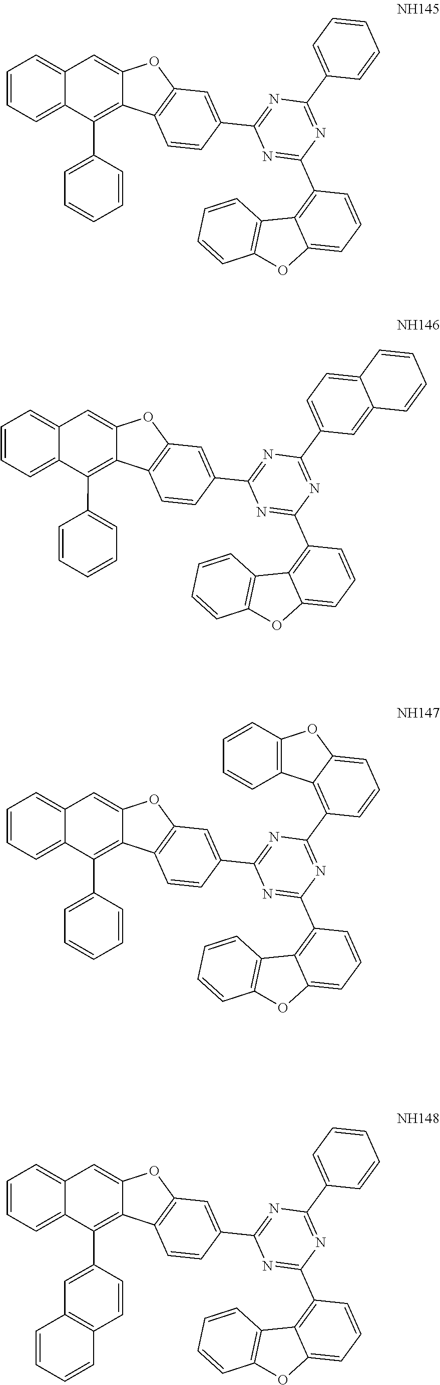 [SMILES: c1ccc(-c2nc(-c3ccc4c(c3)oc3cc5ccccc5c(-c5ccccc5)c34)nc(-c3cccc4oc5ccccc5c34)n2)cc1 ; c1ccc(-c2c3ccccc3cc3oc4cc(-c5nc(-c6ccc7ccccc7c6)nc(-c6cccc7oc8ccccc8c67)n5)ccc4c23)cc1 ; c1ccc(-c2c3ccccc3cc3oc4cc(-c5nc(-c6cccc7oc8ccccc8c67)nc(-c6cccc7oc8ccccc8c67)n5)ccc4c23)cc1 ; c1ccc(-c2nc(-c3ccc4c(c3)oc3cc5ccccc5c(-c5ccc6ccccc6c5)c34)nc(-c3cccc4oc5ccccc5c34)n2)cc1]

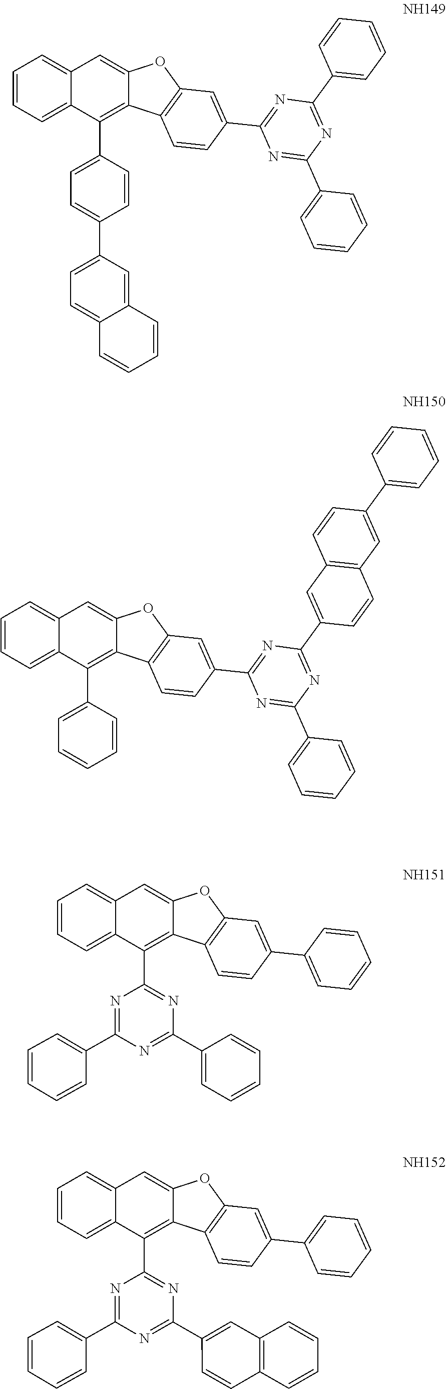 [SMILES: c1ccc(-c2nc(-c3ccccc3)nc(-c3ccc4c(c3)oc3cc5ccccc5c(-c5ccc(-c6ccc7ccccc7c6)cc5)c34)n2)cc1 ; c1ccc(-c2ccc3cc(-c4nc(-c5ccccc5)nc(-c5ccc6c(c5)oc5cc7ccccc7c(-c7ccccc7)c56)n4)ccc3c2)cc1 ; c1ccc(-c2ccc3c(c2)oc2cc4ccccc4c(-c4nc(-c5ccccc5)nc(-c5ccccc5)n4)c23)cc1 ; c1ccc(-c2ccc3c(c2)oc2cc4ccccc4c(-c4nc(-c5ccccc5)nc(-c5ccc6ccccc6c5)n4)c23)cc1]

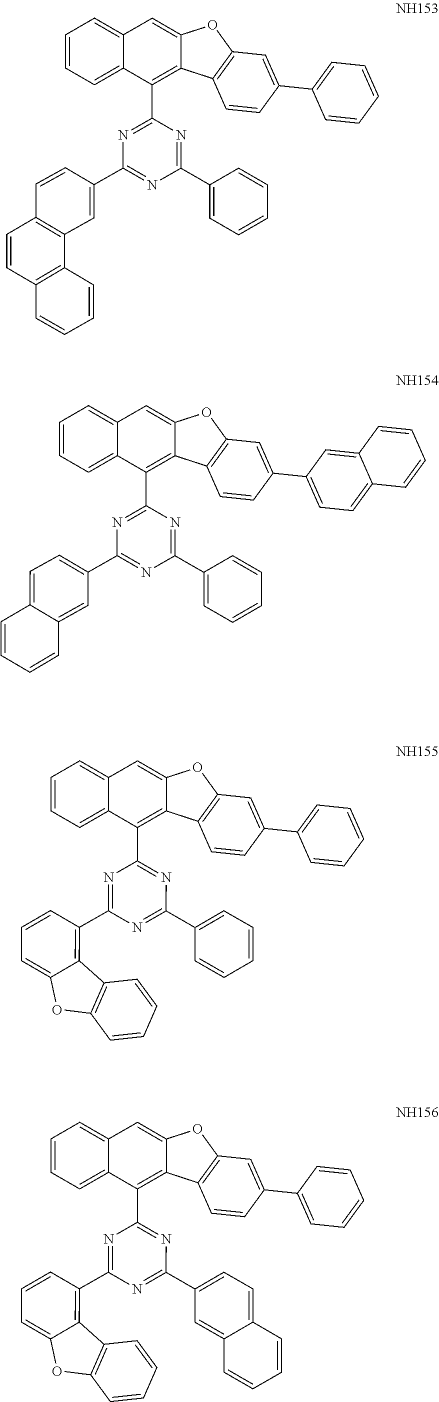 [SMILES: c1ccc(-c2ccc3c(c2)oc2cc4ccccc4c(-c4nc(-c5ccccc5)nc(-c5ccc6ccc7ccccc7c6c5)n4)c23)cc1 ; c1ccc(-c2nc(-c3ccc4ccccc4c3)nc(-c3c4ccccc4cc4oc5cc(-c6ccc7ccccc7c6)ccc5c34)n2)cc1 ; c1ccc(-c2ccc3c(c2)oc2cc4ccccc4c(-c4nc(-c5ccccc5)nc(-c5cccc6oc7ccccc7c56)n4)c23)cc1 ; c1ccc(-c2ccc3c(c2)oc2cc4ccccc4c(-c4nc(-c5ccc6ccccc6c5)nc(-c5cccc6oc7ccccc7c56)n4)c23)cc1]

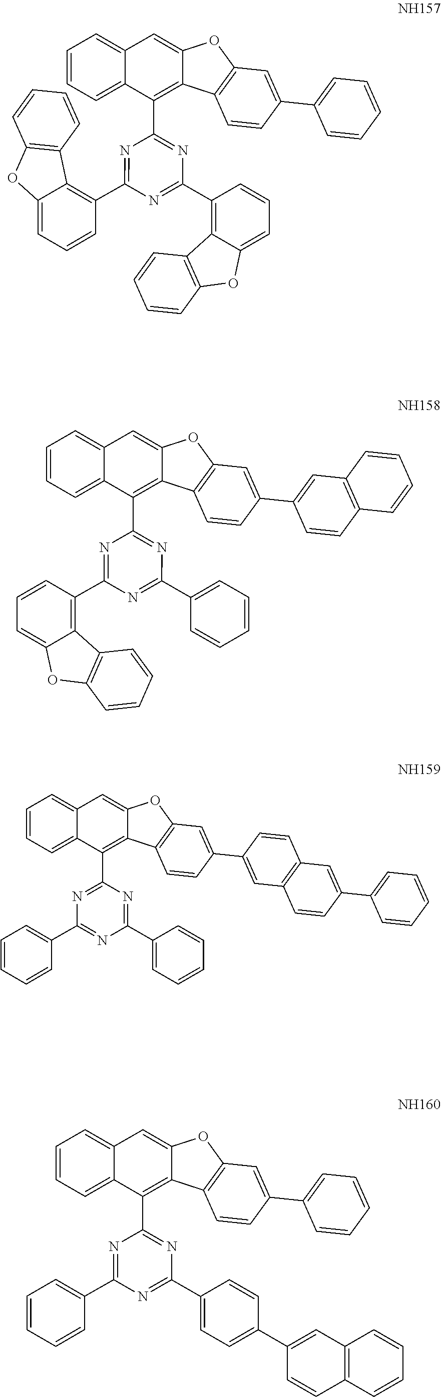 [SMILES: c1ccc(-c2ccc3c(c2)oc2cc4ccccc4c(-c4nc(-c5cccc6oc7ccccc7c56)nc(-c5cccc6oc7ccccc7c56)n4)c23)cc1 ; c1ccc(-c2nc(-c3cccc4oc5ccccc5c34)nc(-c3c4ccccc4cc4oc5cc(-c6ccc7ccccc7c6)ccc5c34)n2)cc1 ; c1ccc(-c2ccc3cc(-c4ccc5c(c4)oc4cc6ccccc6c(-c6nc(-c7ccccc7)nc(-c7ccccc7)n6)c45)ccc3c2)cc1 ; c1ccc(-c2ccc3c(c2)oc2cc4ccccc4c(-c4nc(-c5ccccc5)nc(-c5ccc(-c6ccc7ccccc7c6)cc5)n4)c23)cc1]

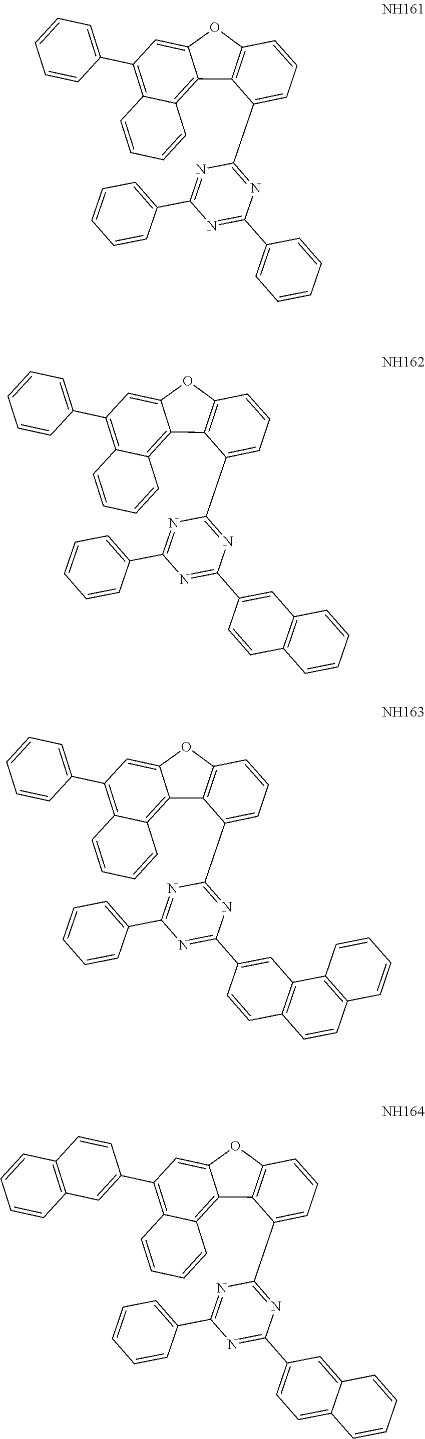 [SMILES: c1ccc(-c2nc(-c3ccccc3)nc(-c3cccc4oc5cc(-c6ccccc6)c6ccccc6c5c34)n2)cc1 ; c1ccc(-c2nc(-c3ccc4ccccc4c3)nc(-c3cccc4oc5cc(-c6ccccc6)c6ccccc6c5c34)n2)cc1 ; c1ccc(-c2nc(-c3ccc4ccc5ccccc5c4c3)nc(-c3cccc4oc5cc(-c6ccccc6)c6ccccc6c5c34)n2)cc1 ; c1ccc(-c2nc(-c3ccc4ccccc4c3)nc(-c3cccc4oc5cc(-c6ccc7ccccc7c6)c6ccccc6c5c34)n2)cc1]

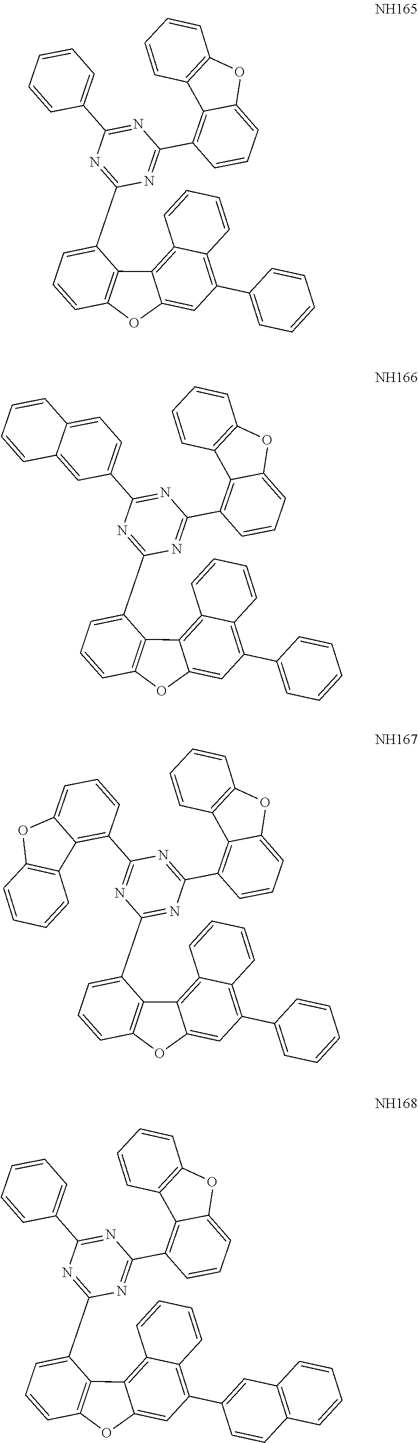 [SMILES: c1ccc(-c2nc(-c3cccc4oc5ccccc5c34)nc(-c3cccc4oc5cc(-c6ccccc6)c6ccccc6c5c34)n2)cc1 ; c1ccc(-c2cc3oc4cccc(-c5nc(-c6ccc7ccccc7c6)nc(-c6cccc7oc8ccccc8c67)n5)c4c3c3ccccc23)cc1 ; c1ccc(-c2cc3oc4cccc(-c5nc(-c6cccc7oc8ccccc8c67)nc(-c6cccc7oc8ccccc8c67)n5)c4c3c3ccccc23)cc1 ; c1ccc(-c2nc(-c3cccc4oc5ccccc5c34)nc(-c3cccc4oc5cc(-c6ccc7ccccc7c6)c6ccccc6c5c34)n2)cc1]

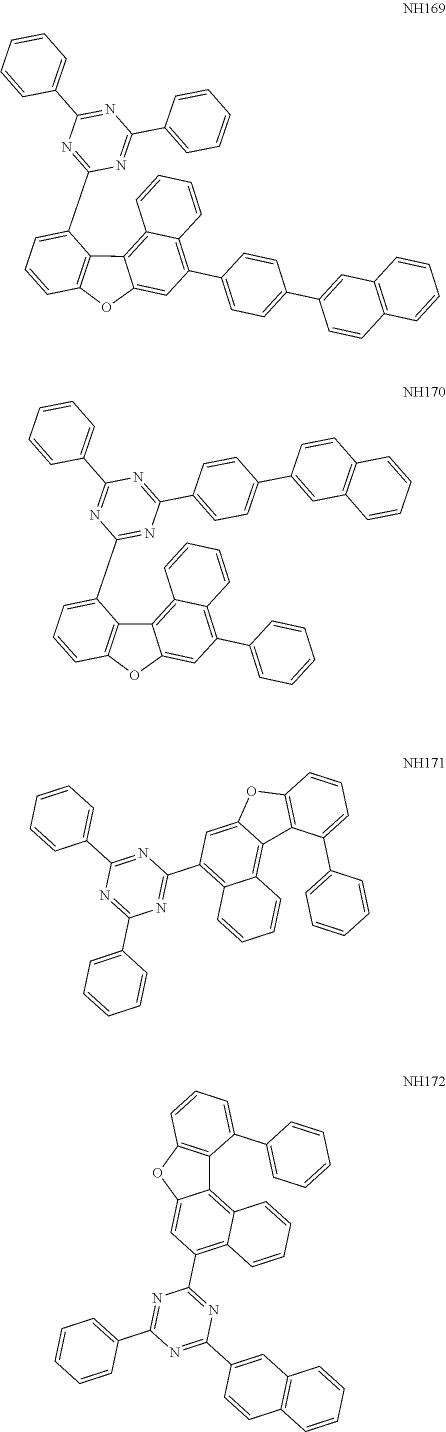 [SMILES: c1ccc(-c2nc(-c3ccccc3)nc(-c3cccc4oc5cc(-c6ccc(-c7ccc8ccccc8c7)cc6)c6ccccc6c5c34)n2)cc1 ; c1ccc(-c2nc(-c3ccc(-c4ccc5ccccc5c4)cc3)nc(-c3cccc4oc5cc(-c6ccccc6)c6ccccc6c5c34)n2)cc1 ; c1ccc(-c2nc(-c3ccccc3)nc(-c3cc4oc5cccc(-c6ccccc6)c5c4c4ccccc34)n2)cc1 ; c1ccc(-c2nc(-c3ccc4ccccc4c3)nc(-c3cc4oc5cccc(-c6ccccc6)c5c4c4ccccc34)n2)cc1]

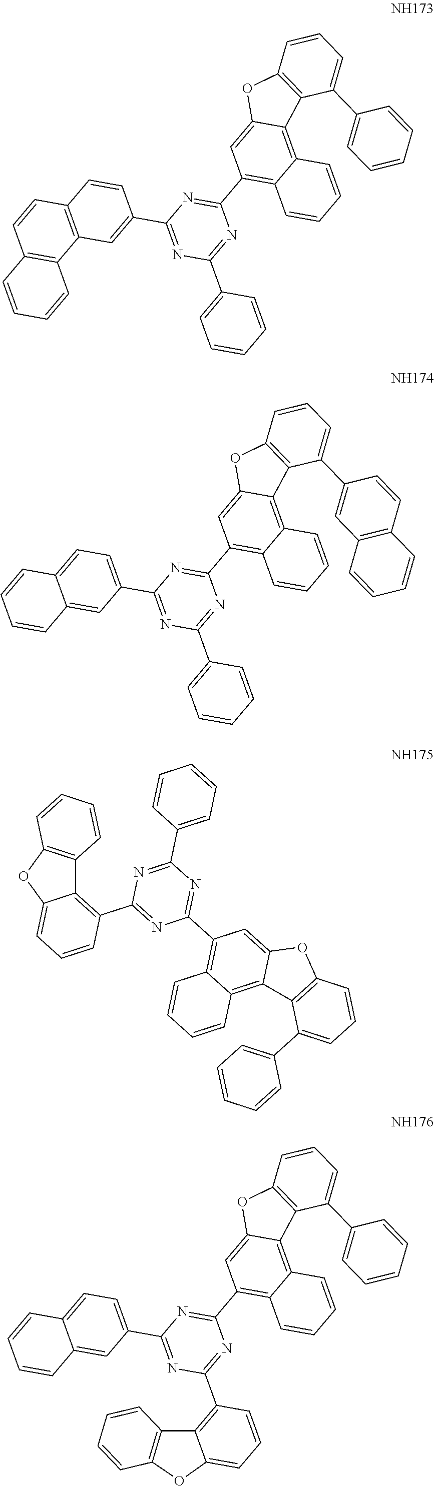

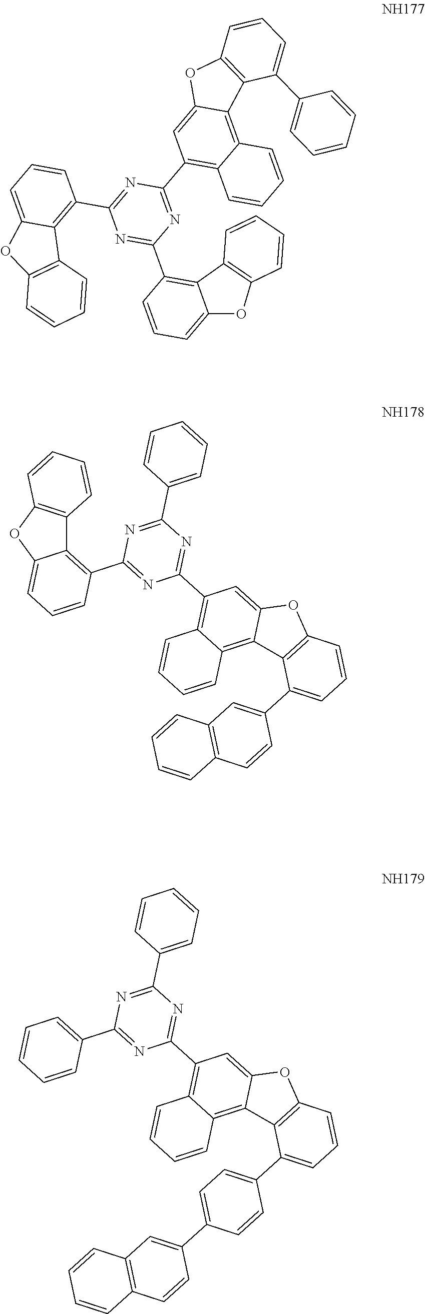 [SMILES: c1ccc(-c2cccc3oc4cc(-c5nc(-c6cccc7oc8ccccc8c67)nc(-c6cccc7oc8ccccc8c67)n5)c5ccccc5c4c23)cc1 ; c1ccc(-c2nc(-c3cc4oc5cccc(-c6ccc7ccccc7c6)c5c4c4ccccc34)nc(-c3cccc4oc5ccccc5c34)n2)cc1 ; c1ccc(-c2nc(-c3ccccc3)nc(-c3cc4oc5cccc(-c6ccc(-c7ccc8ccccc8c7)cc6)c5c4c4ccccc34)n2)cc1]

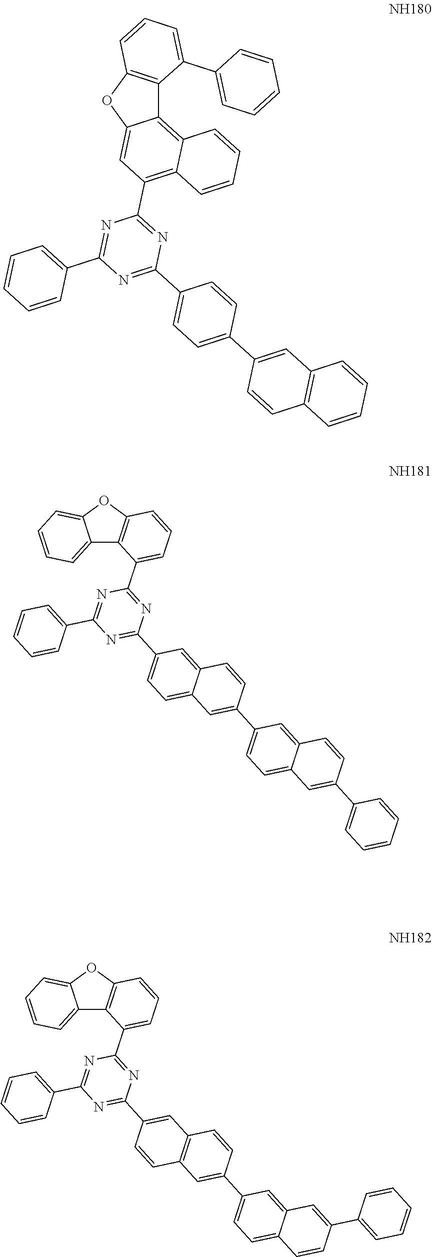 [SMILES: c1ccc(-c2nc(-c3ccc(-c4ccc5ccccc5c4)cc3)nc(-c3cc4oc5cccc(-c6ccccc6)c5c4c4ccccc34)n2)cc1 ; c1ccc(-c2ccc3cc(-c4ccc5cc(-c6nc(-c7ccccc7)nc(-c7cccc8oc9ccccc9c78)n6)ccc5c4)ccc3c2)cc1 ; c1ccc(-c2ccc3ccc(-c4ccc5cc(-c6nc(-c7ccccc7)nc(-c7cccc8oc9ccccc9c78)n6)ccc5c4)cc3c2)cc1]

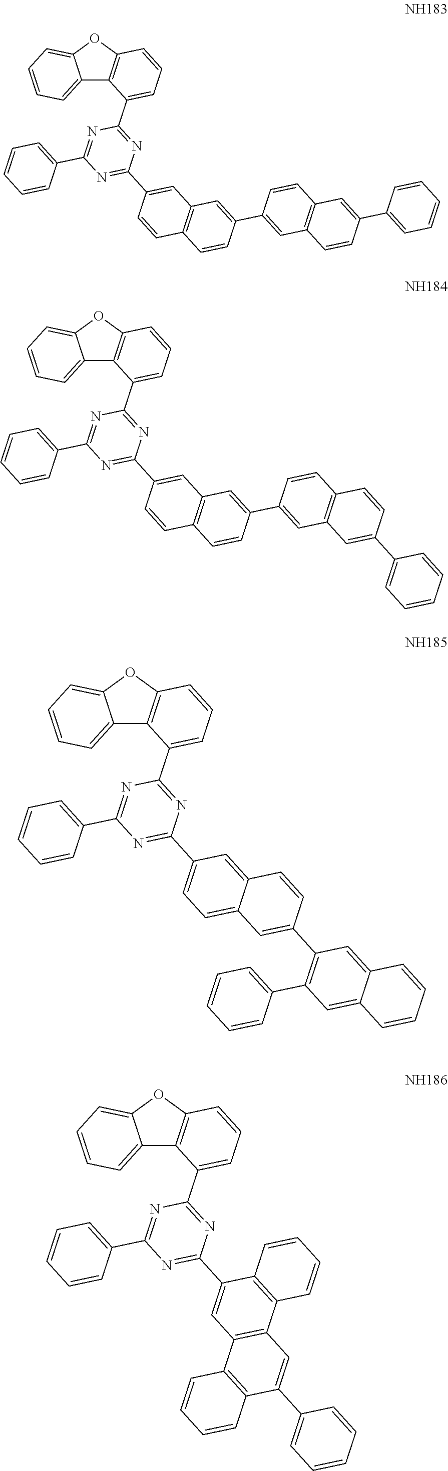 [SMILES: c1ccc(-c2ccc3cc(-c4ccc5ccc(-c6nc(-c7ccccc7)nc(-c7cccc8oc9ccccc9c78)n6)cc5c4)ccc3c2)cc1 ; c1ccc(-c2ccc3ccc(-c4ccc5ccc(-c6nc(-c7ccccc7)nc(-c7cccc8oc9ccccc9c78)n6)cc5c4)cc3c2)cc1 ; c1ccc(-c2nc(-c3ccc4cc(-c5cc6ccccc6cc5-c5ccccc5)ccc4c3)nc(-c3cccc4oc5ccccc5c34)n2)cc1 ; c1ccc(-c2nc(-c3cc4c5ccccc5c(-c5ccccc5)cc4c4ccccc34)nc(-c3cccc4oc5ccccc5c34)n2)cc1]

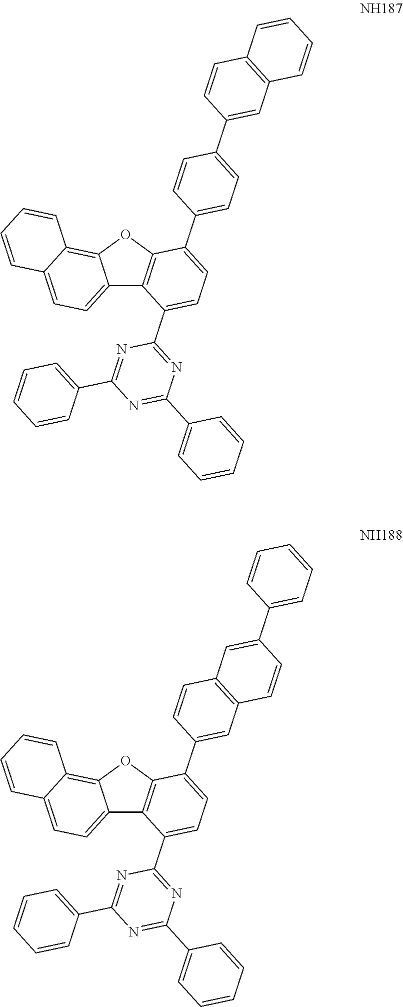 [SMILES: c1ccc(-c2nc(-c3ccccc3)nc(-c3ccc(-c4ccc(-c5ccc6ccccc6c5)cc4)c4oc5c6ccccc6ccc5c34)n2)cc1 ; c1ccc(-c2ccc3cc(-c4ccc(-c5nc(-c6ccccc6)nc(-c6ccccc6)n5)c5c4oc4c6ccccc6ccc45)ccc3c2)cc1]

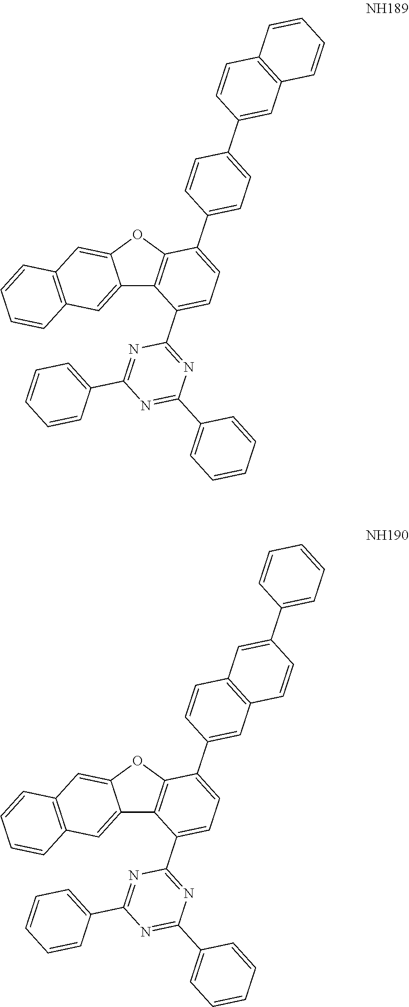 [SMILES: c1ccc(-c2nc(-c3ccccc3)nc(-c3ccc(-c4ccc(-c5ccc6ccccc6c5)cc4)c4oc5cc6ccccc6cc5c34)n2)cc1 ; c1ccc(-c2ccc3cc(-c4ccc(-c5nc(-c6ccccc6)nc(-c6ccccc6)n5)c5c4oc4cc6ccccc6cc45)ccc3c2)cc1]

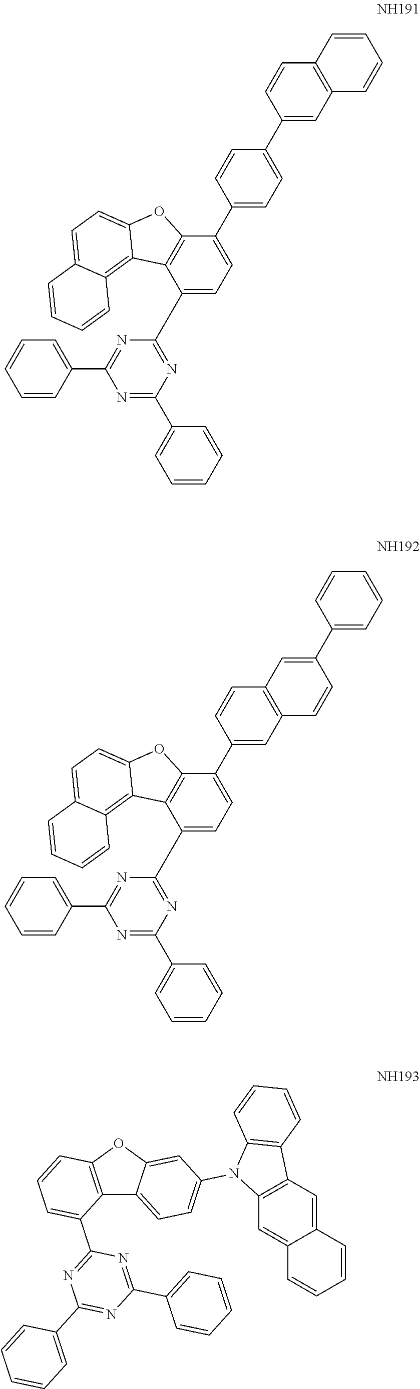 [SMILES: c1ccc(-c2nc(-c3ccccc3)nc(-c3ccc(-c4ccc(-c5ccc6ccccc6c5)cc4)c4oc5ccc6ccccc6c5c34)n2)cc1 ; c1ccc(-c2ccc3cc(-c4ccc(-c5nc(-c6ccccc6)nc(-c6ccccc6)n5)c5c4oc4ccc6ccccc6c45)ccc3c2)cc1 ; c1ccc(-c2nc(-c3ccccc3)nc(-c3cccc4oc5cc(-n6c7ccccc7c7cc8ccccc8cc76)ccc5c34)n2)cc1]

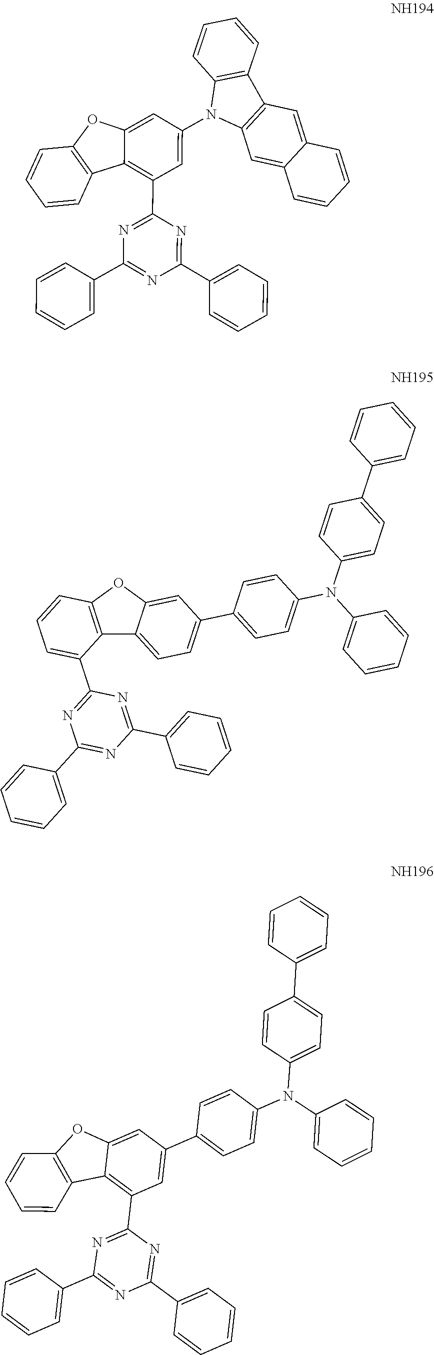 [SMILES: c1ccc(-c2nc(-c3ccccc3)nc(-c3cc(-n4c5ccccc5c5cc6ccccc6cc54)cc4oc5ccccc5c34)n2)cc1 ; c1ccc(-c2ccc(N(c3ccccc3)c3ccc(-c4ccc5c(c4)oc4cccc(-c6nc(-c7ccccc7)nc(-c7ccccc7)n6)c45)cc3)cc2)cc1 ; c1ccc(-c2ccc(N(c3ccccc3)c3ccc(-c4cc(-c5nc(-c6ccccc6)nc(-c6ccccc6)n5)c5c(c4)oc4ccccc45)cc3)cc2)cc1]

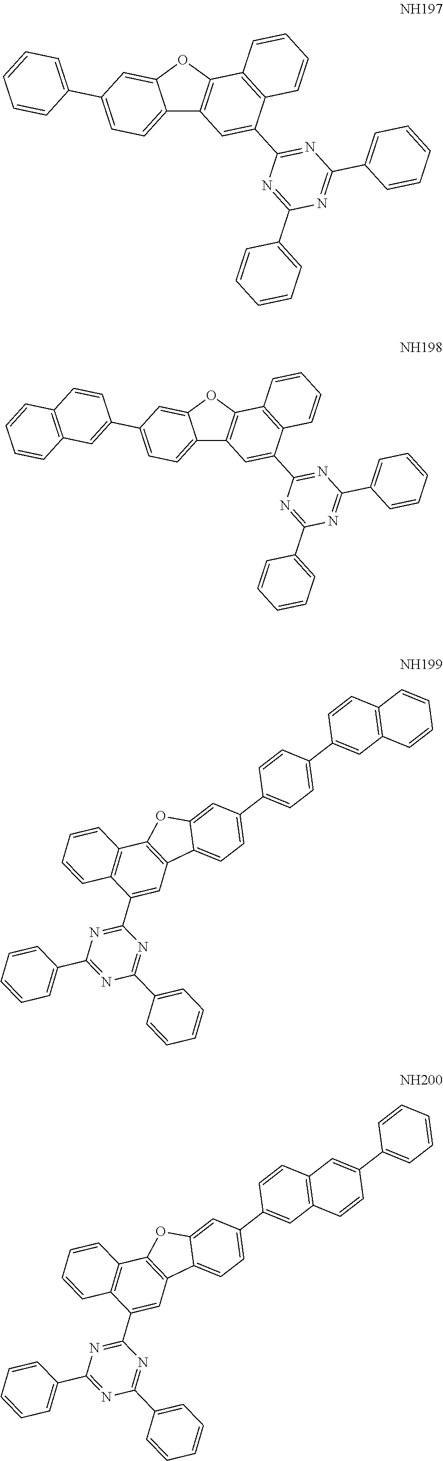 [SMILES: c1ccc(-c2ccc3c(c2)oc2c4ccccc4c(-c4nc(-c5ccccc5)nc(-c5ccccc5)n4)cc32)cc1 ; c1ccc(-c2nc(-c3ccccc3)nc(-c3cc4c5ccc(-c6ccc7ccccc7c6)cc5oc4c4ccccc34)n2)cc1 ; c1ccc(-c2nc(-c3ccccc3)nc(-c3cc4c5ccc(-c6ccc(-c7ccc8ccccc8c7)cc6)cc5oc4c4ccccc34)n2)cc1 ; c1ccc(-c2ccc3cc(-c4ccc5c(c4)oc4c6ccccc6c(-c6nc(-c7ccccc7)nc(-c7ccccc7)n6)cc54)ccc3c2)cc1]

When the heterocyclic compound represented by Chemical Formula 1 and the heterocyclic compound represented by Chemical Formula 2 are included at the same time, effects of more superior efficiency and lifetime are obtained. From this, it may be expected that an exciplex phenomenon occurs when the two compounds are included at the same time.

The exciplex phenomenon is a phenomenon of releasing energy having sizes of a donor (p-host) HOMO energy level and an acceptor (n-host) LUMO energy level due to electron exchanges between two molecules. When the exciplex phenomenon occurs between two molecules, reverse intersystem crossing (RISC) occurs, and as a result, internal quantum efficiency of fluorescence may increase up to 100%. When a donor (p-host) having a favorable hole transport ability and an acceptor (n-host) having a favorable electron transport ability are used as a host of a light emitting layer, holes are injected to the p-host and electrons are injected to the n-host, and thus a driving voltage may be lowered, which resultantly helps with enhancement in the lifetime. In other words, when the compound represented by Chemical Formula 1 is used as the donor and the compound represented by Chemical Formula 2 is used as the acceptor, excellent device properties are obtained.

In one embodiment of the present disclosure, when the heterocyclic compound represented by Chemical Formula 1 and the heterocyclic compound represented by Chemical Formula 2 are included at the same time, at least one of the compounds may not include deuterium as a substituent, or may have a deuterium content of greater than 0%, 1% or greater, 10% or greater, 20% or greater, 30% or greater, 40% or greater or 50% or greater, and 100% or less, 90% or less, 80% or less, 70% or less or 60% or less with respect to the total number of hydrogen atoms and deuterium atoms.

In another embodiment of the present disclosure, at least one of the compounds may not include deuterium as a substituent, or may have a deuterium content of 1% to 100% with respect to the total number of hydrogen atoms and deuterium atoms.

In another embodiment of the present disclosure, at least one of the compounds may not include deuterium as a substituent, or may have a deuterium content of 20% to 90% with respect to the total number of hydrogen atoms and deuterium atoms.

In another embodiment of the present disclosure, at least one of the compounds may not include deuterium as a substituent, or may have a deuterium content of 30% to 80% with respect to the total number of hydrogen atoms and deuterium atoms.

In another embodiment of the present disclosure, at least one of the compounds may not include deuterium as a substituent, or may have a deuterium content of 50% to 70% with respect to the total number of hydrogen atoms and deuterium atoms.

In addition, one embodiment of the present disclosure provides a composition for an organic material layer, the composition including: the heterocyclic compound represented by Chemical Formula 1; and the heterocyclic compound represented by Chemical Formula 2.

Specific descriptions on the heterocyclic compound represented by Chemical Formula 1 and the heterocyclic compound represented by Chemical Formula 2 are the same as the descriptions provided above.

In one embodiment of the present disclosure, the heterocyclic compound represented by Chemical Formula 1 and the heterocyclic compound represented by Chemical Formula 2 may have a weight ratio of 1:9 to 9:1, 1:9 to 5:5 or 2:8 to 5:5 in the composition for an organic material layer, however, the ratio is not limited thereto.

The composition for an organic material layer may be used when forming an organic material of an organic light emitting device, and particularly, may be more preferably used when forming a host of a light emitting layer.

In one embodiment of the present disclosure, the organic material layer includes the heterocyclic compound represented by Chemical Formula 1 and the heterocyclic compound represented by Chemical

Formula 2, and a phosphorescent dopant may be used therewith.

As the phosphorescent dopant material, those known in the art may be used. For example, phosphorescent dopant materials represented by LL′MX′, LL′L″M, LMX′X″, L₂MX′ and L₃M may be used, however, the scope of the present disclosure is not limited by these examples.

M may be iridium, platinum, osmium or the like.

L is an anionic bidentate ligand coordinated to M by sp² carbon and heteroatom, and X may function to trap electrons or holes. Nonlimiting examples of L may include 2-(1-naphthyl)benzoxazole, 2-phenylbenzoxazole, 2-phenylbenzothiazole, 7,8-benzoquinoline, phenylpyridine, benzothiophenylpyridine, 3-methoxy-2-phenylpyridine, thiophenylpyridine, tolylpyridine and the like. Nonlimiting examples of X′ and X″ may include acetylacetonate (acac), hexafluoroacetylacetonate, salicylidene, picolinate, 8-hydroxyquinolinate and the like.

Specific examples of the phosphorescent dopant are shown below, however, the phosphorescent dopant is not limited to these examples.

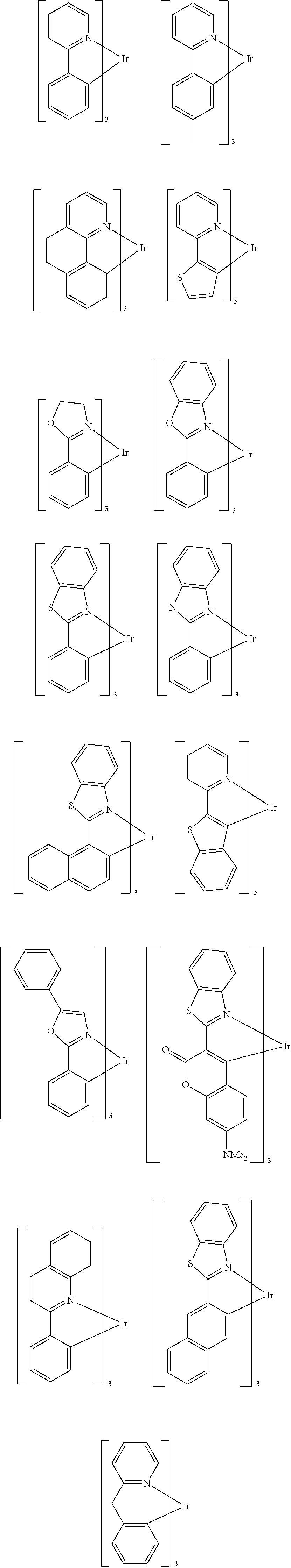

In one embodiment of the present disclosure, the organic material layer includes the heterocyclic compound represented by Chemical Formula 1 and the heterocyclic compound represented by Chemical Formula 2, and an iridium-based dopant may be used therewith.

In one embodiment of the present disclosure, as the iridium-based dopant, (piq)₂(Ir)(acac) may be used as a red phosphorescent dopant, or Ir(ppy)₃ may be used as a green phosphorescent dopant.

In one embodiment of the present disclosure, the content of the dopant may be from 1% to 15%, preferably from 2% to 10% and more preferably from 3% to 7% based on the total weight of the light emitting layer.

In one embodiment of the present disclosure, the organic material layer includes a light emitting layer, and the light emitting layer may include the heterocyclic compound represented by Chemical Formula 1 and the heterocyclic compound represented by Chemical Formula 2.

In another embodiment of the present disclosure, the organic material layer includes a light emitting layer, the light emitting layer may include two or more host materials. At least one of the host materials may include the heterocyclic compound represented by Chemical Formula 1, and another one may include the heterocyclic compound represented by Chemical Formula 2.

In another embodiment of the present disclosure, two or more host materials may be pre-mixed and used in the light emitting layer. At least one of the two or more host materials may include the heterocyclic compound represented by Chemical Formula 1, and another one may include the heterocyclic compound represented by Chemical Formula 2.

The pre-mixing means, before depositing the two or more host materials on the organic material layer, putting and mixing the materials first in one source of supply.

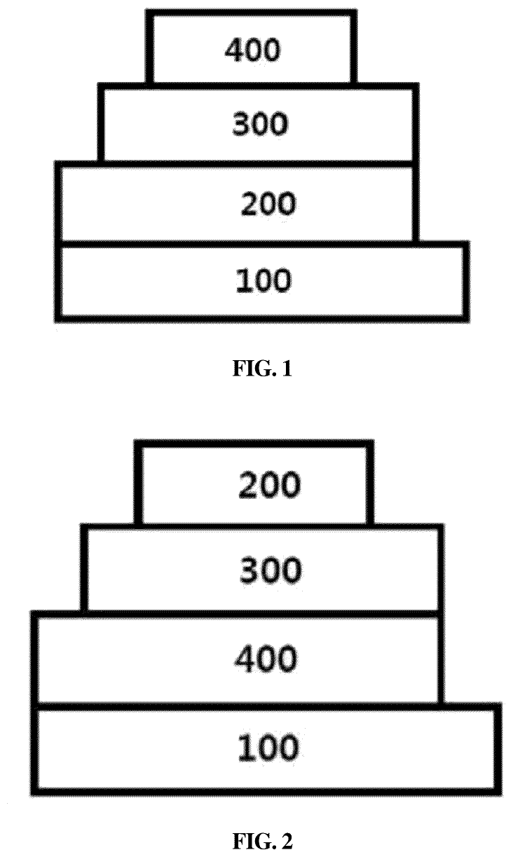

FIGS. 1 to 3 illustrate a lamination order of electrodes and organic material layers of the organic light emitting device according to one embodiment of the present disclosure. However, it is not intended that the scope of the present application be limited by these drawings, and structures of organic light emitting devices known in the art may also be applied to the present application.

FIG. 1 illustrates an organic light emitting device in which a positive electrode 200, an organic material layer 300 and a negative electrode 400 are sequentially laminated on a substrate 100. However, the structure is not limited only to such a structure, and as illustrated in FIG. 2, an organic light emitting device in which a negative electrode, an organic material layer and a positive electrode are sequentially laminated on a substrate may also be obtained.

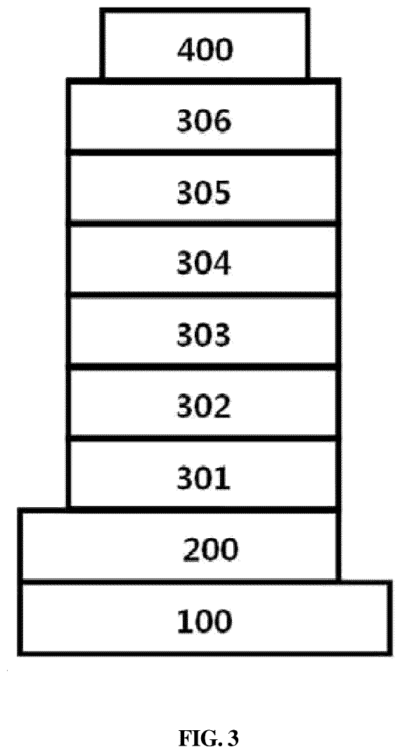

FIG. 3 illustrates a case of the organic material layer being a multilayer. An organic light emitting device according to FIG. 3 includes a hole injection layer 301, a hole transport layer 302, a light emitting layer 303, a hole blocking layer 304, an electron transport layer 305 and an electron injection layer 306. However, the scope of the present application is not limited by such a lamination structure, and as necessary, the layers other than the light emitting layer may not be included, and other necessary functional layers may be further added.

One embodiment of the present disclosure provides a method for manufacturing an organic light emitting device, the method comprising: preparing a substrate; forming a first electrode on the substrate; forming one or more organic material layers on the first electrode; and forming a second electrode on the one or more organic material layers, wherein the forming of one or more organic material layers comprises forming the one or more organic material layers using the composition for an organic material layer according to one embodiment of the present disclosure.

In one embodiment of the present disclosure, the forming of organic material layers may be forming the organic material layers using a thermal vacuum deposition method after pre-mixing the heterocyclic compound represented by Chemical Formula 1 and the heterocyclic compound represented by Chemical Formula 2.

The pre-mixing means, before depositing the heterocyclic compound represented by Chemical Formula 1 and the heterocyclic compound represented by Chemical Formula 2 on the organic material layer, putting and mixing the materials first in one source of supply.

The pre-mixed material may be referred to as the composition for an organic material layer according to one embodiment of the present application.

The organic material layer comprising the heterocyclic compound represented by Chemical Formula 1 may further include other materials as necessary.

The organic material layer comprising the heterocyclic compound represented by Chemical Formula 1 and the heterocyclic compound represented by Chemical Formula 2 at the same time may further include other materials as necessary.

In the organic light emitting device according to one embodiment of the present disclosure, materials other than the heterocyclic compound represented by Chemical Formula 1 and the heterocyclic compound represented by Chemical Formula 2 are illustrated below, however, these are for illustrative purposes only and not for limiting the scope of the present application, and these materials may be replaced by materials known in the art.

As the positive electrode material, materials each having a relatively large work function may be used, and transparent conductive oxides, metals, conductive polymers or the like may be used. Specific examples of the positive electrode material include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene] (PEDOT), polypyrrole and polyaniline, and the like, but are not limited thereto.

As the negative electrode material, materials each having a relatively small work function may be used, and metals, metal oxides, conductive polymers or the like may be used. Specific examples of the negative electrode material include metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection layer material, known hole injection layer materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tris[phenyl(m-tolyl)amino]triphenylamine (m-MTDATA) or 1,3,5-tris[4-(3-methylphenylphenylamino)phenyl]benzene (m-MTDAPB) described in the literature [Advanced Material, 6, p. 677 (1994)], conductive polymers having solubility such as polyaniline/dodecylbenzenesulfonic acid or poly(3,4-ethylenedioxythiophene)/poly(4-styrenesulfonate), polyaniline/camphor sulfonic acid or polyaniline/poly(4-styrenesulfonate), and the like, may be used.

As the hole transport layer material, pyrazoline derivatives, arylamine-based derivatives, stilbene derivatives, triphenyldiamine derivatives and the like may be used, and low molecular or high molecular materials may also be used.

As the electron transport layer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials as well as low molecular materials may also be used.

As examples of the electron injection layer material, LiF is typically used in the art, however, the present application is not limited thereto.

As the light emitting layer material, red, green or blue light emitting materials may be used, and as necessary, two or more light emitting materials may be mixed and used. Herein, the two or more light emitting materials may be deposited as individual sources of supply or pre-mixed and deposited as one source of supply when used. In addition, fluorescent materials may also be used as the light emitting layer material, however, phosphorescent materials may also be used. As the light emitting layer material, materials emitting light alone by binding holes and electrons injected from a positive electrode and a negative electrode, respectively, may be used, however, materials having a host material and a dopant material involved in light emission together may also be used.

When hosts of the light emitting layer material are mixed and used, same series hosts may be mixed and used, or different series hosts may be mixed and used. For example, any two or more types of materials among n-type host materials and p-type host materials may be selected and used as a host material of a light emitting layer.

The organic light emitting device according to one embodiment of the present disclosure may be a top-emission type, a bottom-emission type or a dual-emission type depending on the materials used.

The heterocyclic compound according to one embodiment of the present disclosure may also be used in an organic electronic device including an organic solar cell, an organic photo conductor, an organic transistor and the like under a principle similar to that in the organic light emitting device.

Hereinafter, preferred examples are provided to help to understand the present disclosure, however, the following examples are only provided to more readily understand the present disclosure, and the present disclosure is not limited thereto.

### PREPARATION EXAMPLE

### Preparation Example 1. Preparation of Compound 2

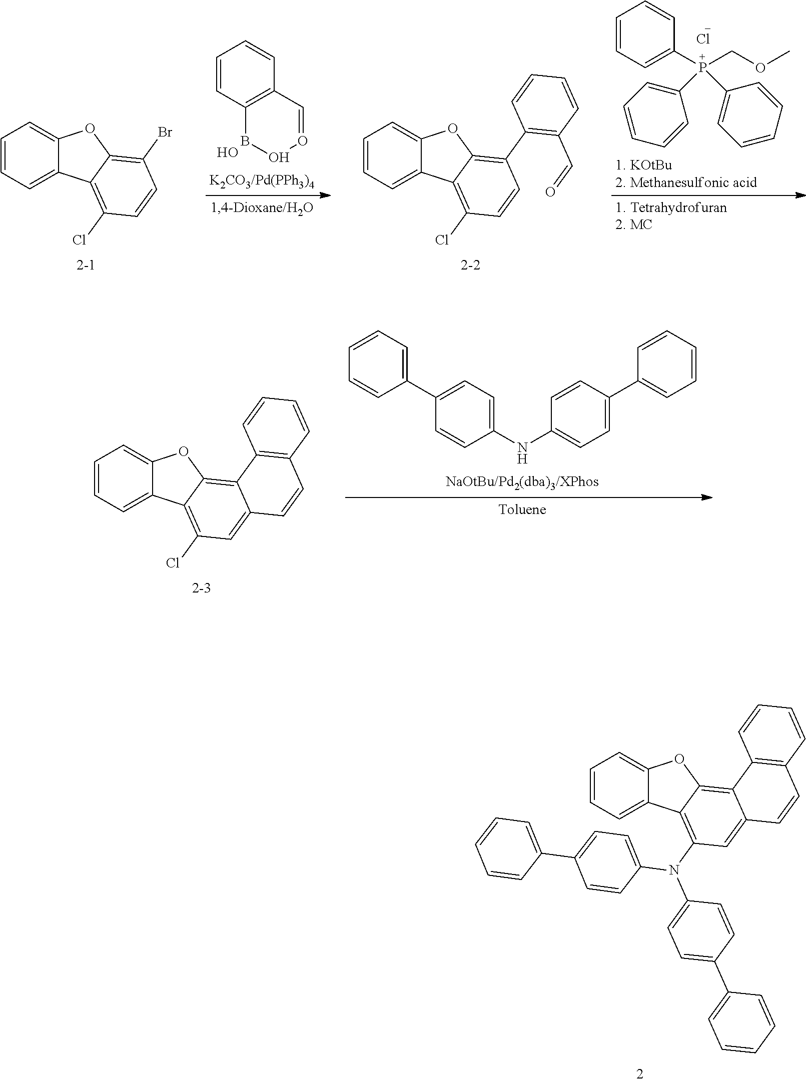

### Preparation Example 1-1. Preparation of Compound 2-2

Compound 2-1 (30 g, 0.107 mol, 1 eq.), (2-formylphenyl)boronic acid (20.8 g, 0.139 mol, 1.3 eq.), K₂CO₃ (36.8 g, 0.266 mol, 2.5 eq.) and Pd(PPh₃)₄ (3.7 g, 0.003 mol, 0.03 eq.) were introduced to 1,4-dioxane (330 mL) and water (60 mL), and the mixture was stirred for 6 hours at 100° C.

Water was added thereto to terminate the reaction, then the reaction solution was extracted with methylene chloride and water, and moisture was removed using MgSO₄. After that, the result was separated using a silica gel column to obtain Compound 2-2 (25 g, yield 76%).

### Preparation Example 1-2. Preparation of Compound 2-3

Compound 2-2 (25 g, 0.082 mol, 1 eq.), (methoxymethyl)triphenylphosphonium chloride (100 g, 0.285 mol, 3.5 eq.) and KOt-Bu (32 g, 0.285 mol, 3.5 eq.) were introduced to tetrahydrofuran (250 mL), and the mixture was stirred for 3 hours at room temperature.

Water was added thereto to terminate the reaction, then the reaction solution was extracted with methylene chloride and water, and the moisture was removed using MgSO₄. After that, the result was dissolved in methylene chloride (250 mL), methanesulfonic acid (15 mL) was slowly introduced thereto, and the mixture was stirred for 3 hours at room temperature.

Water was added thereto to terminate the reaction, then the reaction solution was extracted with methylene chloride and water, and moisture was removed using MgSO₄. After that, the result was separated using a silica gel column to obtain Compound 2-3 (19 g, yield 77%).

### Preparation Example 1-3. Preparation of Compound 2

Compound 2-3 (4.8 g, 0.016 mol, 1 eq.), di([1,1′-biphenyl]-4-yl)amine (5.2 g, 0.016 mol, 1 eq.), NaOt-Bu (3 g, 0.032 mol, 2 eq.), Pd₂(dba)₃ (0.58 g, 0.0006 mol, 0.04 eq.) and XPhos (0.6 g, 0.0013 mol, 0.08 eq.) were introduced to xylene (100 mL), and the mixture was stirred for 3 hours at 130° C.

Water was added thereto to terminate the reaction, then the reaction solution was extracted with methylene chloride and water, and moisture was removed using MgSO₄. After that, the result was separated using a silica gel column to obtain Compound 2 (6 g, yield 64%).

### Preparation Example 2. Preparation of Compound 16

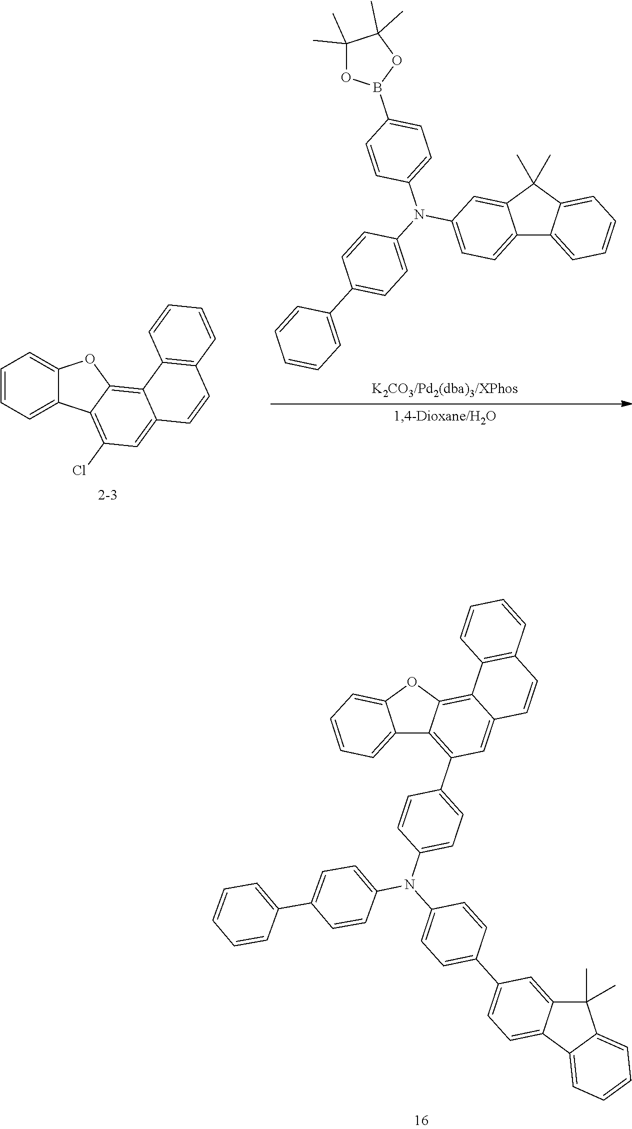 [SMILES: Clc1cc2ccc3ccccc3c2c2oc3ccccc3c12 ; CC1(C)c2ccccc2-c2ccc(N(c3ccc(B4OC(C)(C)C(C)(C)O4)cc3)c3ccc(-c4ccccc4)cc3)cc21 ; CC1(C)c2ccccc2-c2ccc(-c3ccc(N(c4ccc(-c5ccccc5)cc4)c4ccc(-c5cc6ccc7ccccc7c6c6oc7ccccc7c56)cc4)cc3)cc21]

Compound 2-3 was obtained in the same manner as in Preparation Examples 1-1 and 1-2.

Compound 2-3 (4.8 g, 0.016 mol, 1 eq.), N-([1,1′-biphenyl]-4-yl)-9,9-dimethyl-N-(4 (4,4,5,5-tetramethyl-1,3,2-dioxaborolan-2-yl)phenyl)-9H-fluoren-2-amine (8.9 g, 0.016 mol, 1 eq.), K₂CO₃ (4.4 g, 0.032 mol, 2 eq.), Pd₂(dba)₃ (0.58 g, 0.0006 mol, 0.04 eq.) and XPhos (0.6 g, 0.0013 mol, 0.08 eq.) were introduced to 1,4-dioxane (100 mL) and water (25 mL), and the mixture was stirred for 3 hours at 90° C.

Water was added thereto to terminate the reaction, then the reaction solution was extracted with methylene chloride and water, and moisture was removed using MgSO₄. After that, the result was separated using a silica gel column to obtain Compound 16 (7 g, yield 63%).

Target compounds were prepared as in the following Table 1 in the same manner as in Preparation Example 1 or 2, except that Compound A of the following Table 1 was used instead of Compound 2-1 in Preparation Example 1, Compound B of the following Table 1 was used instead of (2-formylphenyl)boronic acid in Preparation Example 1, and Compound C of the following Table 1 was used instead of di([1,1′-biphenyl]-4-yl)amine in Preparation Example 1 or Compound C of the following Table 1 was used instead of N-([1,1′-biphenyl]-4-yl)-9,9-dimethyl-N-(4-(4,4,5,5-tetramethyl-1,3,2-dioxaborolan-2-yl)phenyl)-9H-fluoren-2-amine in Preparation Example 2.

TABLE 1

| Com- | | |
|---|---|---|
| pound | | |
| No. | Compound A | Compound B |
| 27 | 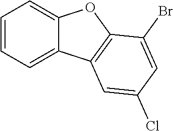 [SMILES: Clc1cc(Br)c2oc3ccccc3c2c1] | 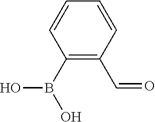 [SMILES: O=Cc1ccccc1B(O)O] |
| 50 | 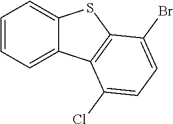 [SMILES: Clc1ccc(Br)c2sc3ccccc3c12] | 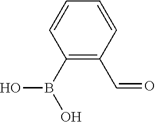 [SMILES: O=Cc1ccccc1B(O)O] |
| 71 | 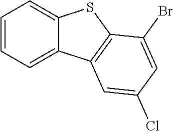 [SMILES: Clc1cc(Br)c2sc3ccccc3c2c1] | 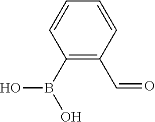 [SMILES: O=Cc1ccccc1B(O)O] |
| 95 | 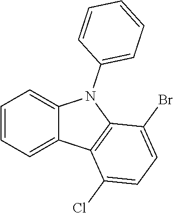 [SMILES: Clc1ccc(Br)c2c1c1ccccc1n2-c1ccccc1] | 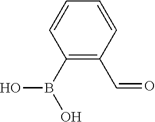 [SMILES: O=Cc1ccccc1B(O)O] |
| 117 | 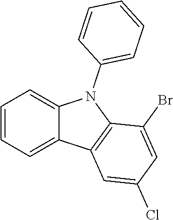 [SMILES: Clc1cc(Br)c2c(c1)c1ccccc1n2-c1ccccc1] | 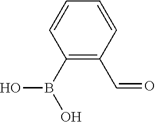 [SMILES: O=Cc1ccccc1B(O)O] |
| 133 | 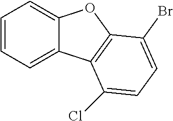 [SMILES: Clc1ccc(Br)c2oc3ccccc3c12] | 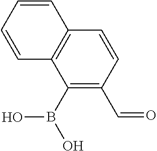 [SMILES: O=Cc1ccc2ccccc2c1B(O)O] |
| 144 | 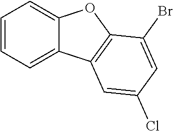 [SMILES: Clc1cc(Br)c2oc3ccccc3c2c1] | 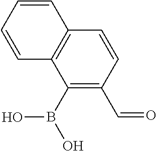 [SMILES: O=Cc1ccc2ccccc2c1B(O)O] |
| 165 | 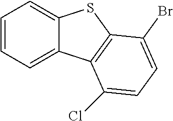 [SMILES: Clc1ccc(Br)c2sc3ccccc3c12] | 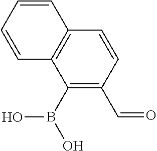 [SMILES: O=Cc1ccc2ccccc2c1B(O)O] |
| 189 | 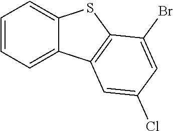 [SMILES: Clc1cc(Br)c2sc3ccccc3c2c1] | 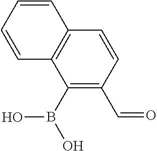 [SMILES: O=Cc1ccc2ccccc2c1B(O)O] |
| 203 | 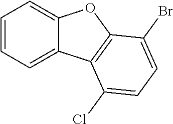 [SMILES: Clc1ccc(Br)c2oc3ccccc3c12] | 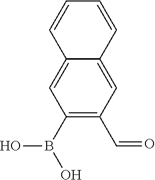 [SMILES: O=Cc1cc2ccccc2cc1B(O)O] |
| 226 | 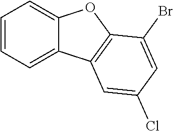 [SMILES: Clc1cc(Br)c2oc3ccccc3c2c1] | 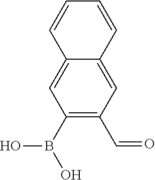 [SMILES: O=Cc1cc2ccccc2cc1B(O)O] |
| 258 | 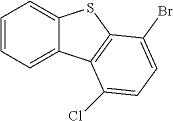 [SMILES: Clc1ccc(Br)c2sc3ccccc3c12] | 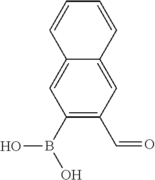 [SMILES: O=Cc1cc2ccccc2cc1B(O)O] |
| 262 | 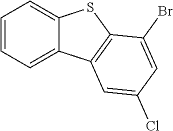 [SMILES: Clc1cc(Br)c2sc3ccccc3c2c1] | 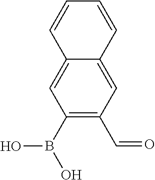 [SMILES: O=Cc1cc2ccccc2cc1B(O)O] |
| 292 | 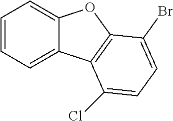 [SMILES: Clc1ccc(Br)c2oc3ccccc3c12] | 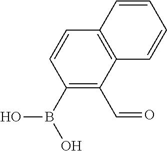 [SMILES: O=Cc1c(B(O)O)ccc2ccccc12] |
| 314 | 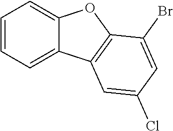 [SMILES: Clc1cc(Br)c2oc3ccccc3c2c1] | 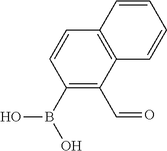 [SMILES: O=Cc1c(B(O)O)ccc2ccccc12] |
| 340 | 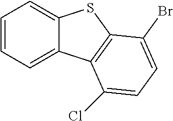 [SMILES: Clc1ccc(Br)c2sc3ccccc3c12] | 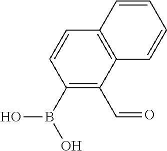 [SMILES: O=Cc1c(B(O)O)ccc2ccccc12] |
| 343 | 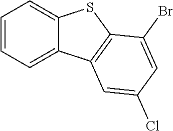 [SMILES: Clc1cc(Br)c2sc3ccccc3c2c1] | 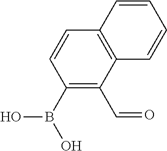 [SMILES: O=Cc1c(B(O)O)ccc2ccccc12] |
| Com- | | |
| pound | | |
| No. | Compound C | Target Compound |
| 27 | 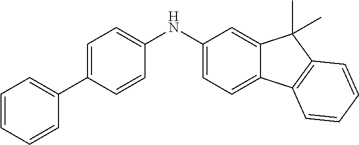 [SMILES: CC1(C)c2ccccc2-c2ccc(Nc3ccc(-c4ccccc4)cc3)cc21] | 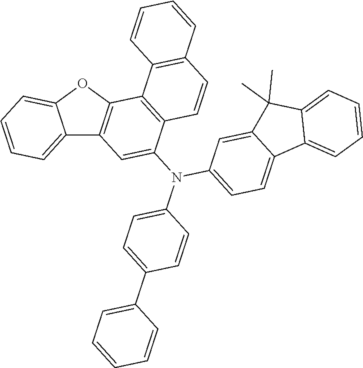 [SMILES: CC1(C)c2ccccc2-c2ccc(N(c3ccc(-c4ccccc4)cc3)c3cc4c5ccccc5oc4c4c3ccc3ccccc34)cc21] |
| 50 | 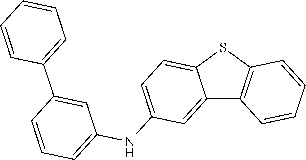 [SMILES: c1ccc(-c2cccc(Nc3ccc4sc5ccccc5c4c3)c2)cc1] | 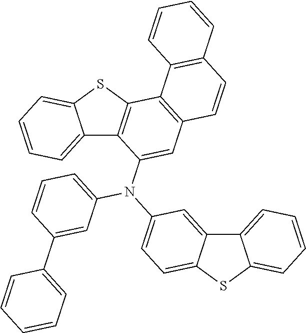 [SMILES: c1ccc(-c2cccc(N(c3ccc4sc5ccccc5c4c3)c3cc4ccc5ccccc5c4c4sc5ccccc5c34)c2)cc1] |
| 71 | 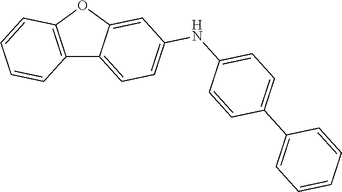 [SMILES: c1ccc(-c2ccc(Nc3ccc4c(c3)oc3ccccc34)cc2)cc1] | 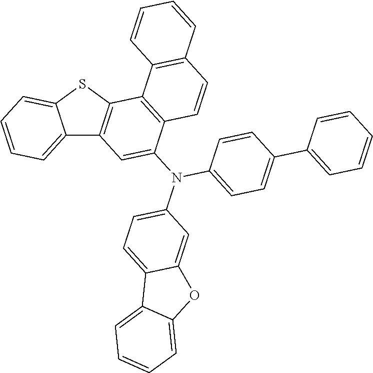 [SMILES: c1ccc(-c2ccc(N(c3ccc4c(c3)oc3ccccc34)c3cc4c5ccccc5sc4c4c3ccc3ccccc34)cc2)cc1] |
| 95 | 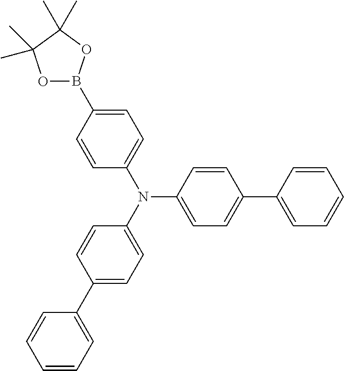 [SMILES: CC1(C)OB(c2ccc(N(c3ccc(-c4ccccc4)cc3)c3ccc(-c4ccccc4)cc3)cc2)OC1(C)C] | 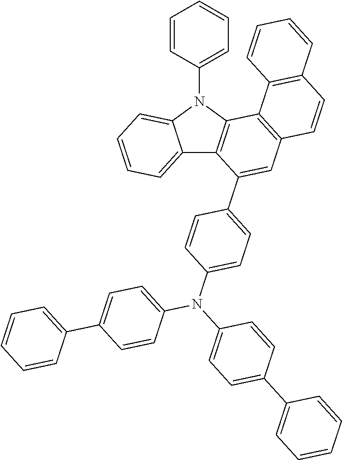 [SMILES: c1ccc(-c2ccc(N(c3ccc(-c4ccccc4)cc3)c3ccc(-c4cc5ccc6ccccc6c5c5c4c4ccccc4n5-c4ccccc4)cc3)cc2)cc1] |
| 117 | 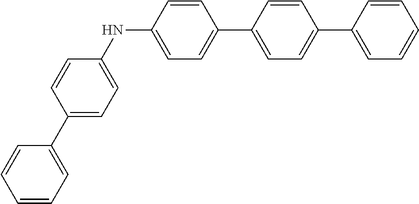 [SMILES: c1ccc(-c2ccc(Nc3ccc(-c4ccc(-c5ccccc5)cc4)cc3)cc2)cc1] | 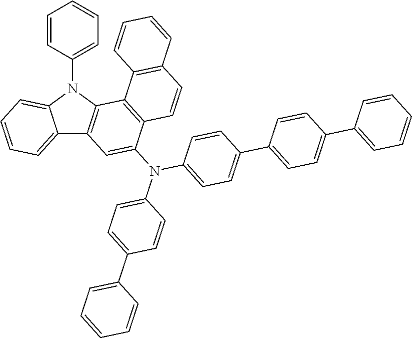 [SMILES: c1ccc(-c2ccc(-c3ccc(N(c4ccc(-c5ccccc5)cc4)c4cc5c6ccccc6n(-c6ccccc6)c5c5c4ccc4ccccc45)cc3)cc2)cc1] |
| 133 | 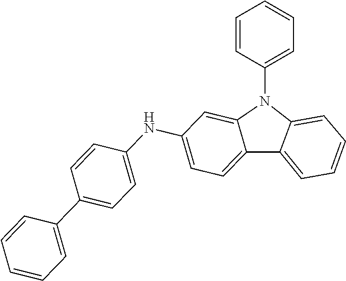 [SMILES: c1ccc(-c2ccc(Nc3ccc4c5ccccc5n(-c5ccccc5)c4c3)cc2)cc1] | 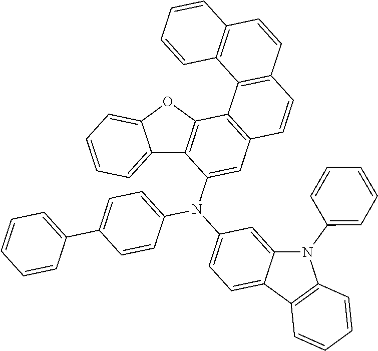 [SMILES: c1ccc(-c2ccc(N(c3ccc4c5ccccc5n(-c5ccccc5)c4c3)c3cc4ccc5ccc6ccccc6c5c4c4oc5ccccc5c34)cc2)cc1] |
| 144 | 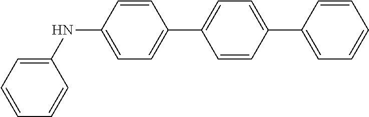 [SMILES: c1ccc(Nc2ccc(-c3ccc(-c4ccccc4)cc3)cc2)cc1] | 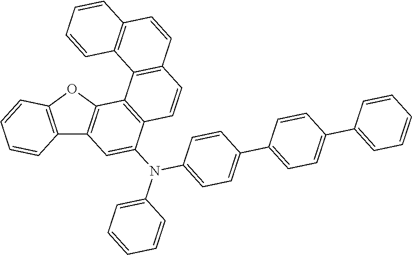 [SMILES: c1ccc(-c2ccc(-c3ccc(N(c4ccccc4)c4cc5c6ccccc6oc5c5c4ccc4ccc6ccccc6c45)cc3)cc2)cc1] |
| 165 | 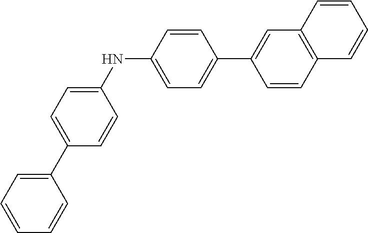 [SMILES: c1ccc(-c2ccc(Nc3ccc(-c4ccc5ccccc5c4)cc3)cc2)cc1] | 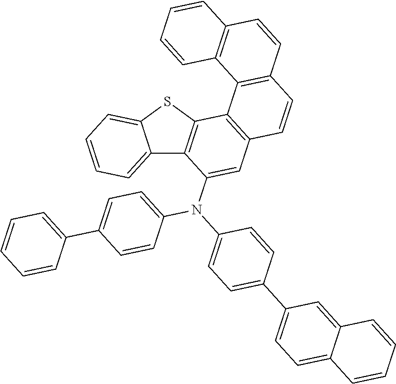 [SMILES: c1ccc(-c2ccc(N(c3ccc(-c4ccc5ccccc5c4)cc3)c3cc4ccc5ccc6ccccc6c5c4c4sc5ccccc5c34)cc2)cc1] |
| 189 | 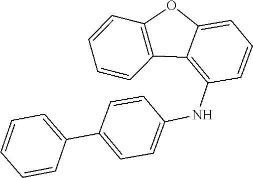 [SMILES: c1ccc(-c2ccc(Nc3cccc4oc5ccccc5c34)cc2)cc1] | 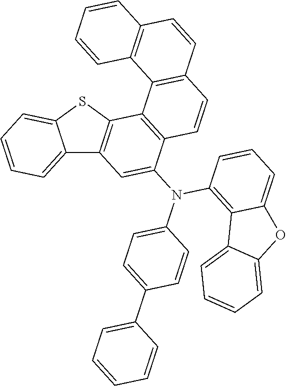 [SMILES: c1ccc(-c2ccc(N(c3cc4c5ccccc5sc4c4c3ccc3ccc5ccccc5c34)c3cccc4oc5ccccc5c34)cc2)cc1] |
| 203 | 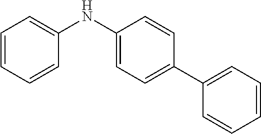 [SMILES: c1ccc(Nc2ccc(-c3ccccc3)cc2)cc1] | 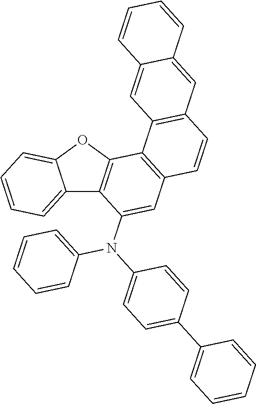 [SMILES: c1ccc(-c2ccc(N(c3ccccc3)c3cc4ccc5cc6ccccc6cc5c4c4oc5ccccc5c34)cc2)cc1] |
| 226 | 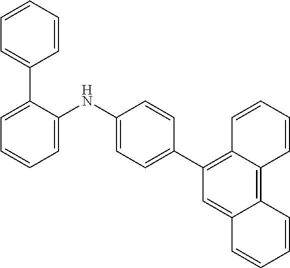 [SMILES: c1ccc(-c2ccccc2Nc2ccc(-c3cc4ccccc4c4ccccc34)cc2)cc1] | 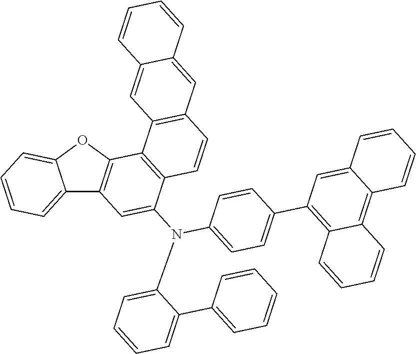 [SMILES: c1ccc(-c2ccccc2N(c2ccc(-c3cc4ccccc4c4ccccc34)cc2)c2cc3c4ccccc4oc3c3c2ccc2cc4ccccc4cc23)cc1] |
| 258 | 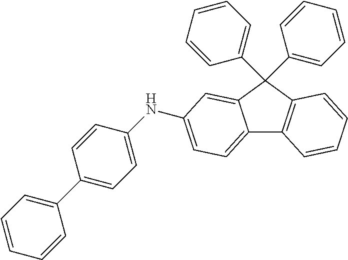 [SMILES: c1ccc(-c2ccc(Nc3ccc4c(c3)C(c3ccccc3)(c3ccccc3)c3ccccc3-4)cc2)cc1] | 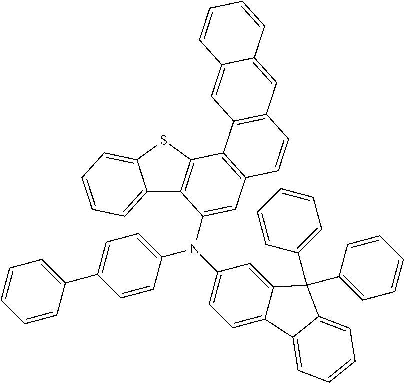 [SMILES: c1ccc(-c2ccc(N(c3ccc4c(c3)C(c3ccccc3)(c3ccccc3)c3ccccc3-4)c3cc4ccc5cc6ccccc6cc5c4c4sc5ccccc5c34)cc2)cc1] |
| 262 | 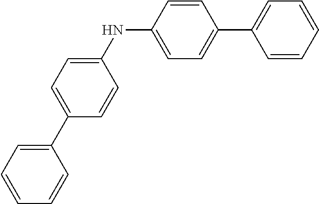 [SMILES: c1ccc(-c2ccc(Nc3ccc(-c4ccccc4)cc3)cc2)cc1] | 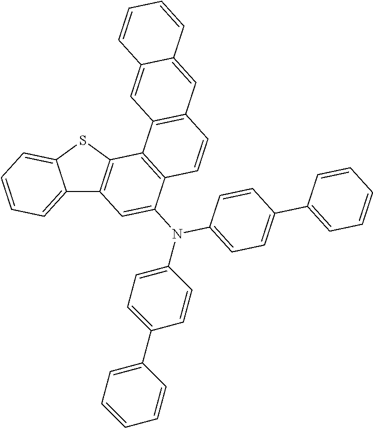 [SMILES: c1ccc(-c2ccc(N(c3ccc(-c4ccccc4)cc3)c3cc4c5ccccc5sc4c4c3ccc3cc5ccccc5cc34)cc2)cc1] |
| 292 | 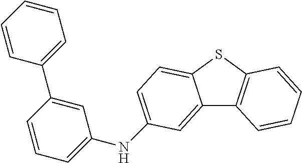 [SMILES: c1ccc(-c2cccc(Nc3ccc4sc5ccccc5c4c3)c2)cc1] | 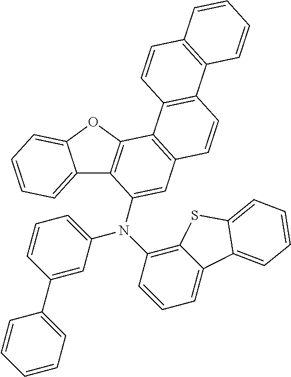 [SMILES: c1ccc(-c2cccc(N(c3cccc4c3sc3ccccc34)c3cc4ccc5c6ccccc6ccc5c4c4oc5ccccc5c34)c2)cc1] |
| 314 | 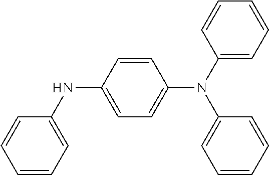 [SMILES: c1ccc(Nc2ccc(N(c3ccccc3)c3ccccc3)cc2)cc1] | 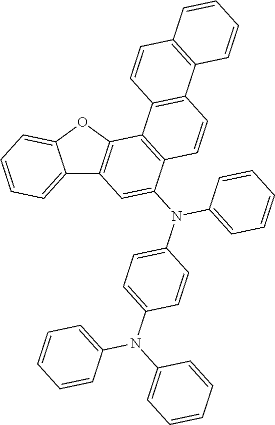 [SMILES: c1ccc(N(c2ccccc2)c2ccc(N(c3ccccc3)c3cc4c5ccccc5oc4c4c3ccc3c5ccccc5ccc34)cc2)cc1] |
| 340 | 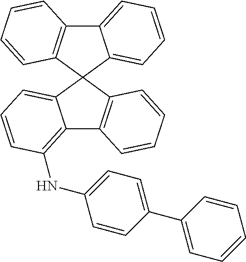 [SMILES: c1ccc(-c2ccc(Nc3cccc4c3-c3ccccc3C43c4ccccc4-c4ccccc43)cc2)cc1] | 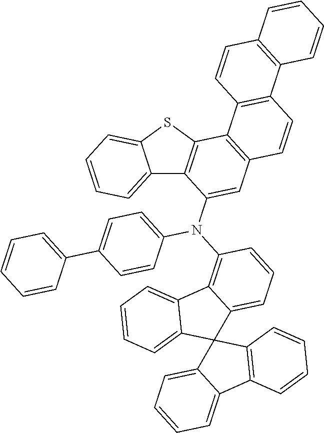 [SMILES: c1ccc(-c2ccc(N(c3cccc4c3-c3ccccc3C43c4ccccc4-c4ccccc43)c3cc4ccc5c6ccccc6ccc5c4c4sc5ccccc5c34)cc2)cc1] |
| 343 | 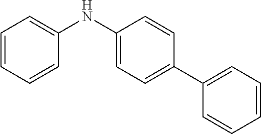 [SMILES: c1ccc(Nc2ccc(-c3ccccc3)cc2)cc1] | 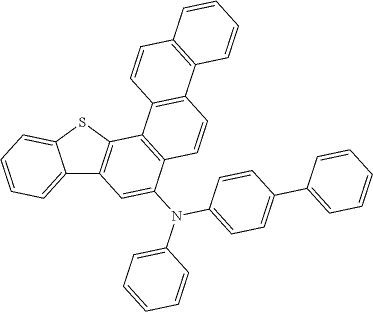 [SMILES: c1ccc(-c2ccc(N(c3ccccc3)c3cc4c5ccccc5sc4c4c3ccc3c5ccccc5ccc34)cc2)cc1] |

### Preparation Example 3. Preparation of Compound 362

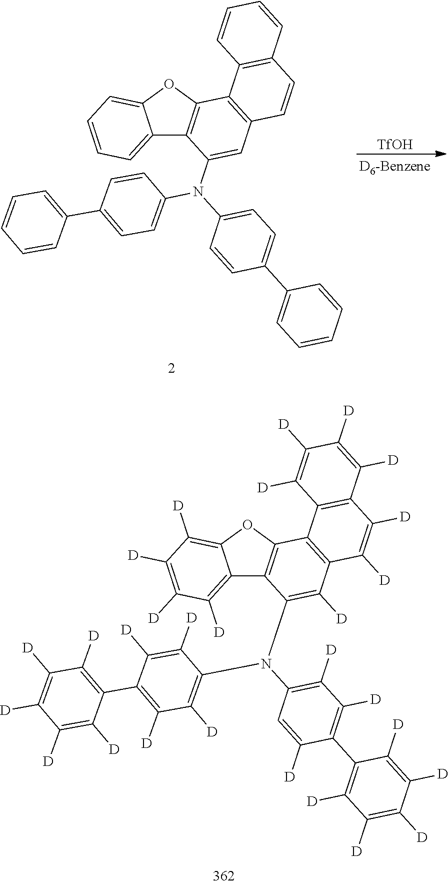

After introducing Compound 2 (7 g, 0.011 mol, 1 eq.), TfOH (2.7 g, 0.018 mol, 1.5 eq.) and Do-benzene (70 mL), the mixture was stirred for 6 hours at 80° C.

Water was added thereto to terminate the reaction, then the reaction solution was extracted with methylene chloride and water, and moisture was removed using MgSO₄. After that, the result was separated using a silica gel column to obtain Compound 203 (6 g, yield 80%).

### Preparation Example 4. Preparation of Compound NH5

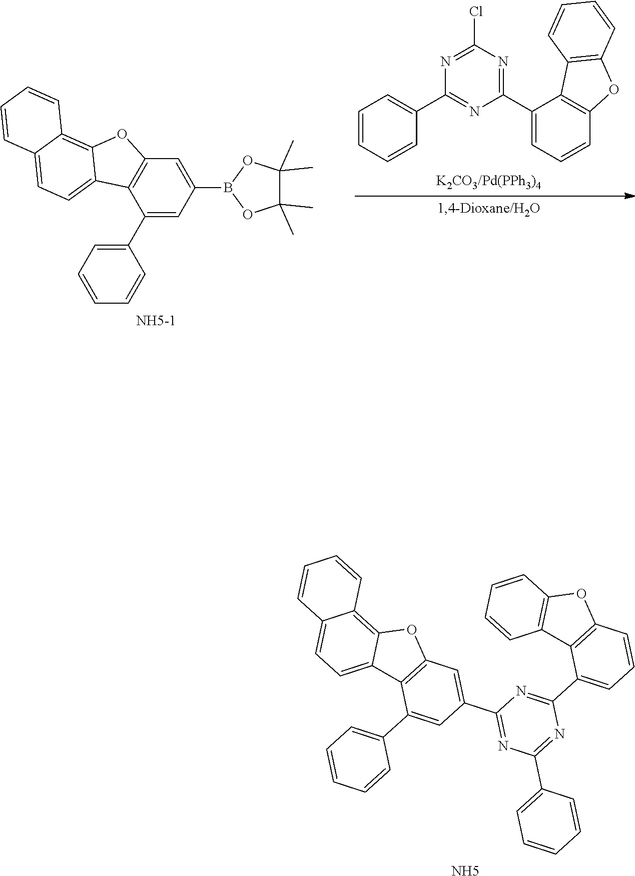

Compound NH5-1 (10 g, 0.024 mol, 1 eq.), 2-chloro-4-(dibenzo[b,d]furan-1-yl)-6-phenyl-1,3,5-triazine (9.4 g, 0.026 mol, 1.1 eq.), K₂CO₃ (9.9 g, 0.071 mol, 3 eq.) and Pd(PPh₃)₄ (1.4 g, 0.001 mol, 0.05 eq.) were introduced to 1,4-dioxane (100 mL) and water (20 mL), and the mixture was stirred for 5 hours at 100° C.

Water was added thereto to terminate the reaction, then the reaction solution was extracted with methylene chloride and water, and moisture was removed using MgSO₄. After that, the result was separated using a silica gel column to obtain Compound NH5 (11 g, yield 75%).

Target compounds were prepared as in the following Table 2 in the same manner as in Preparation Example 4, except that Compound D of the following Table 2 was used instead of Compound NH5-1, and Compound E of the following Table 2 was used instead of 2-chloro-4-(dibenzo[b,d]furan-1-yl)-6-phenyl-1,3,5-triazine.

TABLE 2

| Com- | | | |
|---|---|---|---|
| pound | | | |
| No. | Compound D | Compound E | Target Compound |
| NH30 | 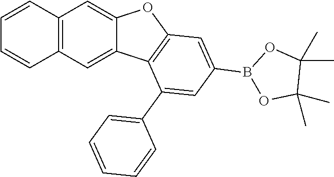 [SMILES: CC1(C)OB(c2cc(-c3ccccc3)c3c(c2)oc2cc4ccccc4cc23)OC1(C)C] | 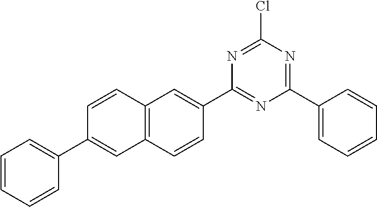 [SMILES: Clc1nc(-c2ccccc2)nc(-c2ccc3cc(-c4ccccc4)ccc3c2)n1] | 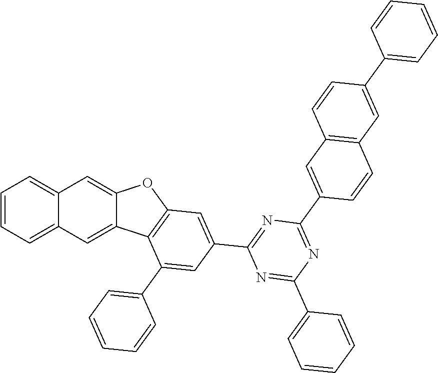 [SMILES: c1ccc(-c2ccc3cc(-c4nc(-c5ccccc5)nc(-c5cc(-c6ccccc6)c6c(c5)oc5cc7ccccc7cc56)n4)ccc3c2)cc1] |
| NH43 | 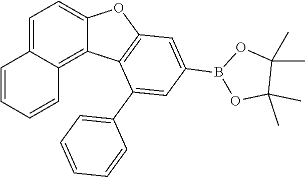 [SMILES: CC1(C)OB(c2cc(-c3ccccc3)c3c(c2)oc2ccc4ccccc4c23)OC1(C)C] | 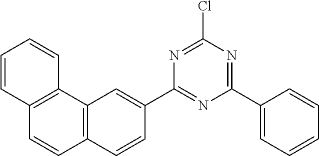 [SMILES: Clc1nc(-c2ccccc2)nc(-c2ccc3ccc4ccccc4c3c2)n1] | 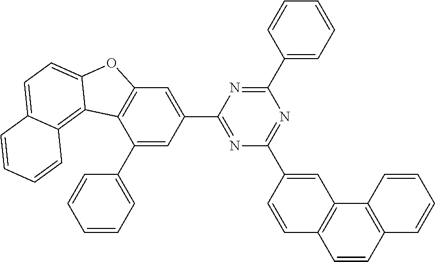 [SMILES: c1ccc(-c2nc(-c3cc(-c4ccccc4)c4c(c3)oc3ccc5ccccc5c34)nc(-c3ccc4ccc5ccccc5c4c3)n2)cc1] |
| NH79 | 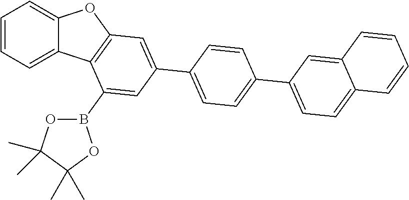 [SMILES: CC1(C)OB(c2cc(-c3ccc(-c4ccc5ccccc5c4)cc3)cc3oc4ccccc4c23)OC1(C)C] | 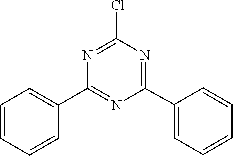 [SMILES: Clc1nc(-c2ccccc2)nc(-c2ccccc2)n1] | 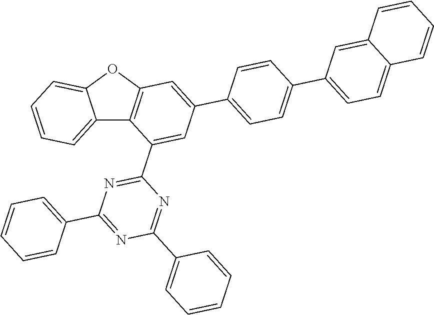 [SMILES: c1ccc(-c2nc(-c3ccccc3)nc(-c3cc(-c4ccc(-c5ccc6ccccc6c5)cc4)cc4oc5ccccc5c34)n2)cc1] |
| NH84 | 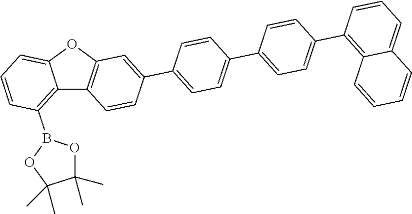 [SMILES: CC1(C)OB(c2cccc3oc4cc(-c5ccc(-c6ccc(-c7cccc8ccccc78)cc6)cc5)ccc4c23)OC1(C)C] | 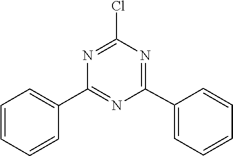 [SMILES: Clc1nc(-c2ccccc2)nc(-c2ccccc2)n1] | 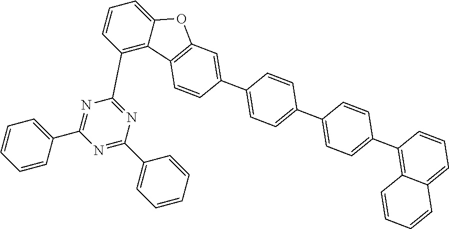 [SMILES: c1ccc(-c2nc(-c3ccccc3)nc(-c3cccc4oc5cc(-c6ccc(-c7ccc(-c8cccc9ccccc89)cc7)cc6)ccc5c34)n2)cc1] |
| NH116 | 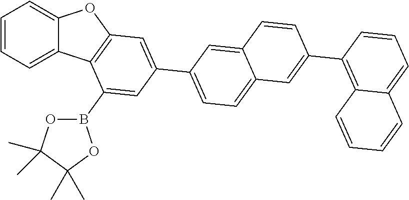 [SMILES: CC1(C)OB(c2cc(-c3ccc4cc(-c5cccc6ccccc56)ccc4c3)cc3oc4ccccc4c23)OC1(C)C] | 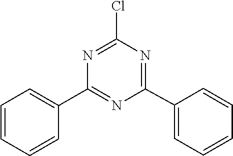 [SMILES: Clc1nc(-c2ccccc2)nc(-c2ccccc2)n1] | 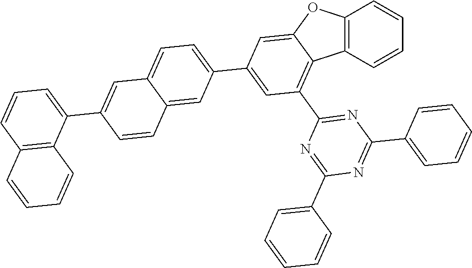 [SMILES: c1ccc(-c2nc(-c3ccccc3)nc(-c3cc(-c4ccc5cc(-c6cccc7ccccc67)ccc5c4)cc4oc5ccccc5c34)n2)cc1] |
| NH128 | 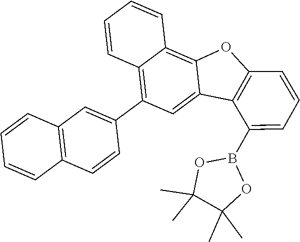 [SMILES: CC1(C)OB(c2cccc3oc4c5ccccc5c(-c5ccc6ccccc6c5)cc4c23)OC1(C)C] | 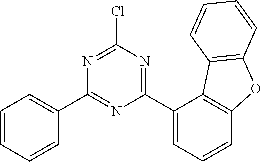 [SMILES: Clc1nc(-c2ccccc2)nc(-c2cccc3oc4ccccc4c23)n1] | 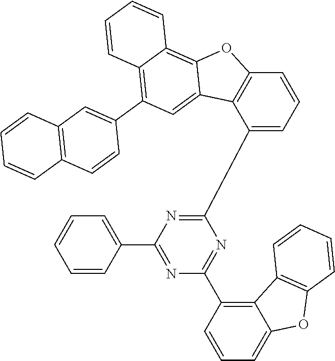 [SMILES: c1ccc(-c2nc(-c3cccc4oc5ccccc5c34)nc(-c3cccc4oc5c6ccccc6c(-c6ccc7ccccc7c6)cc5c34)n2)cc1] |
| NH147 | 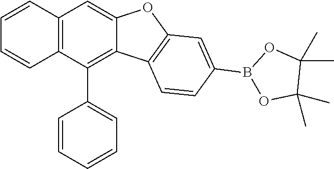 [SMILES: CC1(C)OB(c2ccc3c(c2)oc2cc4ccccc4c(-c4ccccc4)c23)OC1(C)C] | 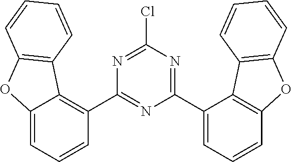 [SMILES: Clc1nc(-c2cccc3oc4ccccc4c23)nc(-c2cccc3oc4ccccc4c23)n1] | 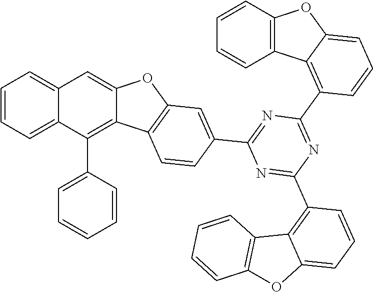 [SMILES: c1ccc(-c2c3ccccc3cc3oc4cc(-c5nc(-c6cccc7oc8ccccc8c67)nc(-c6cccc7oc8ccccc8c67)n5)ccc4c23)cc1] |

Synthesis results for the compounds described in Preparation Examples 1 to 4, and Tables 1 and 2 are shown in the following Tables 3 and 4.

The following 3 shows measurement values of ¹H NMR (CDCl₃, 200 MHz), and the following Table 4 shows measurement values of field desorption mass spectrometry (FD-MS).

TABLE 3

| Compound | |
|---|---|
| No. | ¹H NMR (CDCl₃, 200 MHz) |
| 2 | δ = 8.93(1H, d), 8.12(1H, d), 7.88(3H, m), 7.71(3H, m), 7.54~7.38(17H, m), 6.69(4H, m) |
| 16 | δ = 8.93(1H, d), 8.34(1H, s), 8.12(1H, d), 7.88(4H, m), 7.71~7.28(18H, m), 6.69(5H, m), |
| | 6.58(1H, d), 1.72(6H, s) |
| 27 | δ = 8.93(1H, d), 8.12(1H, d), 7.88(4H, m), 7.71~7.28(16H, m), 7.02(1H, s), 6.69(3H, m) |
| | 6.58(1H, d), 1.72(6H, s) |
| 50 | δ = 8.93(1H, d), 8.45(2H, m), 8.12(1H, d), 7.98(2H, m), 7.88(2H, m), 7.71(3H, m), |
| | 7.52~7.40(11H, m), 7.32(1H, s), 6.88(3H, m) 6.59(1H, d) |
| 71 | δ = 8.93(1H, d), 8.45(1H, d), 8.12(1H, d), 7.98(1H, d), 7.89(3H, m), 7.71(4H, m), |
| | 7.54~7.32(12H, m), 7.02(1H, s), 6.69(2H, m) 6.33(1H, d) |
| 95 | δ = 8.93(1H, d), 8.55(1H, d), 8.34(1H, s), 8.12(1H, d), 7.94(3H, m), 7.71(2H, m), |
| | 7.58~7.25(23H, m), 6.39(6H, m) |
| 117 | δ = 8.93(1H, d), 8.55(1H, d), 8.12(1H, d), 7.94(3H, m), 7.71(2H, m), 7.58~7.25(25H, m), |
| | 7.02(1H, s), 6.39(4H, m) |
| 133 | δ = 8.93(1H, d), 8.12(4H, m), 7.82(4H, m), 7.71~7.29(21H, m), 6.69(2H, m), 6.60(1H, |
| | s), 5.85(1H, d) |
| 144 | δ = 8.93(1H, d), 8.12(2H, m), 7.82(4H, m), 7.71(3H, m), 7.54~7.20(15H, m), 7.02(1H, |
| | s), 6.81(1H, t), 6.69(4H, m) |
| 165 | δ = 8.93(1H, d), 8.45(1H, d), 8.12(2H, m), 8.00(7H, m), 7.71(3H, m), 7.59~7.41(14H, m), |
| | 7.32(1H, s), 6.69(4H, m) |
| 189 | δ = 8.93(1H, d), 8.45(1H, d), 8.12(2H, m), 7.98(1H, d), 7.82(4H, m), 7.71(3H, m), |
| | 7.52~7.38(11H, m), 7.13(1H, t), 7.02(2H, m), 6.69(2H, m), 6.33(1H, d) |
| 203 | δ = 8.31(2H, m), 7.91(4H, m), 7.66(1H, d), 7.54~7.32(13H, m), 7.20(2H, m), 6.81(1H, t), |
| | 6.69(4H, m) |
| 226 | δ = 8.93(2H, m), 8.31(2H, m), 8.12(2H, m), 7.88(9H, m), 7.66(1H, d), 7.54~7.32(11H, |
| | m), 7.16(4H, m), 6.87(1H, t), 6.69(3H, m) |
| 258 | δ = 8.45(1H, d), 8.31(2H, m), 7.98(5H, m), 7.62~7.26(23H, m), 7.11(4H, m), 6.69(3H, |
| | m), 6.58(1H, d) |
| 262 | δ = 8.45(1H, d), 8.31(2H, m), 7.91(4H, m), 7.54~7.39(19H, m), 7.02(1H, s), 6.69(4H, m) |
| 292 | δ = 8.93(2H, m), 8.45(1H, d), 8.12(1H, d), 7.98(1H, d), 7.81(5H, m), 7.71(3H, m), |
| | 7.52~7.32(12H, m), 6.88(3H, m), 6.59(1H, d) |
| 314 | δ = 8.93(2H, m), 8.12(1H, d), 7.88(4H, m), 7.66(3H, m), 7.38(2H, m), 7.20(6H, m), |
| | 7.02(1H, s), 6.81(3H, m), 6.63(6H, m), 6.38(4H, m) |
| 340 | δ = 8.93(2H, m), 8.45(1H, d), 8.12(1H, d), 7.98(1H, d), 7.88(8H, m), 7.52~7.16(19H, m), |
| | 7.03(1H, t), 6.91(1H, d), 6.69(2H, m), 6.58(1H, d) |
| 343 | δ = 8.93(2H, m), 8.45(1H, d), 8.12(1H, d), 7.98(1H, d), 7.82(3H, m), 7.71(2H, m), |
| | 7.50(9H, m), 7.20(2H, m), 7.02(1H, s), 6.81(1H, t), 6.63(4H, m) |
| 362 | — |
| NH5 | δ = 8.28(2H, m), 8.16(2H, m), 7.89(2H, m), 7.75~7.38(19H, m) |
| NH30 | δ = 9.09(1H, s), 8.49(1H, d), 8.28(2H, m), 8.16(2H, m), 7.92(2H, m), 7.73~7.41(21H, m) |
| NH43 | δ = 9.15(1H, s), 8.93(1H, d), 8.54(1H, d), 8.28(2H, m), 8.16(4H, m), 7.88~7.41(18H, m) |
| NH79 | δ = 8.28(4H, m), 7.89(4H, m), 7.73~7.32(19H, m) |
| NH84 | δ = 8.55(1H, d), 8.42(1H, d), 8.28(4H, m), 8.08(2H, m), 7.95(1H, d), 7.75(2H, m), |
| | 7.64(12H, m), 7.25(8H, m) |
| NH116 | δ = 8.55(1H, d), 8.42(1H, d), 8.28(4H, m), 8.04(2H, m), 7.92(3H, m), 7.73~7.32(18H, m) |
| NH128 | δ = 8.55(1H, d), 8.28(2H, m), 8.18(1H, d), 7.89(4H, m), 7.75~7.32(19H, m) |
| NH147 | δ = 8.55(1H, d), 8.08(1H, d), 7.89(3H, m), 7.75~7.32(22H, m) |

TABLE 4

| Compound | |
|---|---|
| No. | FD-MS |
| 2 | m/z = 587.22 |
| 16 | m/z = 703.29 |
| 27 | m/z = 627.26 |
| 50 | m/z = 633.16 |
| 71 | m/z = 617.18 |
| 95 | m/z = 738.30 |
| 117 | m/z = 738.30 |
| 133 | m/z = 726.27 |
| 144 | m/z = 637.24 |
| 165 | m/z = 703.23 |
| 189 | m/z = 667.20 |
| 203 | m/z = 561.21 |
| 226 | m/z = 737.27 |
| 258 | m/z = 817.28 |
| 262 | m/z = 653.22 |
| 292 | m/z = 667.20 |
| 314 | m/z = 625.25 |
| 340 | m/z = 815.26 |
| 343 | m/z = 577.19 |
| 362 | m/z = 616.41 |
| NH5 | m/z = 615.19 |
| NH30 | m/z = 651.23 |
| NH43 | m/z = 625.22 |
| NH79 | m/z = 601.22 |
| NH84 | m/z = 677.25 |
| NH116 | m/z = 651.23 |
| NH128 | m/z = 665.21 |
| NH147 | m/z = 705.21 |

### Experimental Example 1

### Experimental Example 1-1. Manufacture of Organic Light Device

A transparent electrode ITO thin film obtained from glass for an OLED (manufactured by Samsung Corning Advanced Glass) was ultrasonic cleaned using trichloroethylene, acetone, ethanol and distilled water sequentially for 5 minutes each, and then stored in isopropanol before use. Then, the ITO substrate was installed in a substrate folder of a vacuum deposition apparatus.

To a cell in the vacuum deposition apparatus, 4,4′,4″-tris(N,N-(2-naphthyl)-phenylamino)triphenylamine (2-TNATA) was introduced.

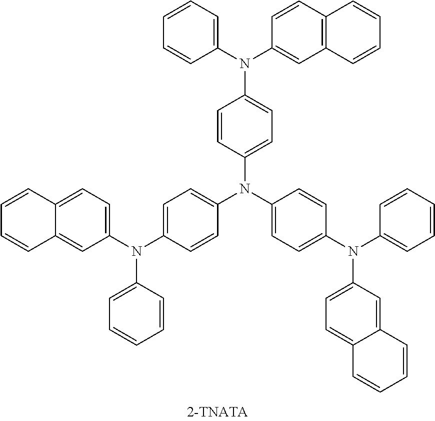 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, the chamber was evacuated until the degree of vacuum therein reached 10⁻⁶ torr, and then the 2-TNATA was evaporated by applying a current to the cell to deposit a hole injection layer having a thickness of 600 Å on the ITO substrate. To another cell in the vacuum deposition apparatus, a compound represented by Chemical Formula 1 or a comparative compound described in the following Table 5 was introduced, and evaporated by applying a current to the cell to deposit a hole transport layer having a thickness of 300 Å on the hole injection layer.

After forming the hole injection layer and the hole transport layer as above, a blue light emitting material having the following structure was deposited thereon as a light emitting layer. Specifically, on one cell in the vacuum deposition apparatus, BH1 that is a blue light emitting host material was vacuum deposited to a thickness of 200 Å, and D1 that is a blue light emitting dopant material was vacuum deposited thereon by 5% with respect to the host material. After that, E1 was deposited to 300 Å as an electron transport layer.

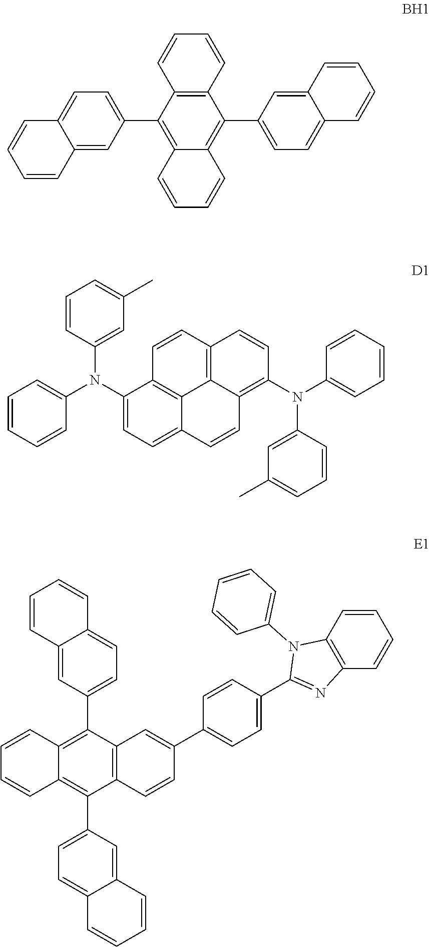

After that, lithium fluoride (LiF) was deposited to a thickness of 10 Å as an electron injection layer, and Al was deposited to a thickness of 1000 Å to form a negative electrode, and as a result, an organic light emitting device was manufactured.

Meanwhile, all the organic compounds required to manufacture the organic light emitting device were vacuum sublimation purified under 108 torr to 106 torr for each material to be used in the manufacture of the organic light emitting device.

### Experimental Example 1-2. Driving Voltage and Light Emission Efficiency of Organic Light Emitting Device

For each of the organic electroluminescent devices manufactured as above, electroluminescent (EL) properties were measured using M7000 manufactured by McScience Inc., and with the measurement results, a lifetime T95 that is a time taken for luminance to become 95% with respect to initial luminance was measured when standard luminance was 700 cd/m² through a lifetime measurement system (M6000) manufactured by McScience Inc.

Results of measuring driving voltage, light emission efficiency and lifetime (T95) of the blue organic light emitting device manufactured according to the manufacturing method are shown in Table 5.

TABLE 5

| | | | Light | | |
|---|---|---|---|---|---|
| | | Driving | Emission | | Life- |
| | | Voltage | Efficiency | CIE | time |
| | Compound | (V) | (cd/A) | (x, y) | (T95) |
| Example 1 | 2 | 4.3 | 6.6 | (0.14, 0.05) | 47 |
| Example 2 | 16 | 4.5 | 6.3 | (0.14, 0.05) | 43 |
| Example 3 | 27 | 4.8 | 6.0 | (0.14, 0.05) | 39 |
| Example 4 | 50 | 4.2 | 6.7 | (0.14, 0.05) | 47 |
| Example 5 | 71 | 4.6 | 6.2 | (0.14, 0.05) | 41 |
| Example 6 | 95 | 4.3 | 6.5 | (0.14, 0.05) | 49 |
| Example 7 | 117 | 4.6 | 6.1 | (0.14, 0.05) | 42 |
| Example 8 | 133 | 4.4 | 6.6 | (0.14, 0.05) | 48 |
| Example 9 | 144 | 4.4 | 6.7 | (0.14, 0.05) | 46 |
| Example 10 | 165 | 4.2 | 6.7 | (0.14, 0.05) | 49 |
| Example 11 | 189 | 4.3 | 6.8 | (0.14, 0.05) | 46 |
| Example 12 | 203 | 4.4 | 6.6 | (0.14, 0.05) | 48 |
| Example 13 | 226 | 4.7 | 6.2 | (0.14, 0.05) | 40 |
| Example 14 | 258 | 4.6 | 6.2 | (0.14, 0.05) | 41 |
| Example 15 | 262 | 4.3 | 6.6 | (0.14, 0.05) | 47 |
| Example 16 | 292 | 4.3 | 6.5 | (0.14, 0.05) | 47 |
| Example 17 | 314 | 4.2 | 6.8 | (0.14, 0.05) | 49 |
| Example 18 | 340 | 4.7 | 6.1 | (0.14, 0.05) | 42 |
| Example 19 | 343 | 4.4 | 6.7 | (0.14, 0.05) | 46 |
| Example 20 | 362 | 4.3 | 6.5 | (0.14, 0.05) | 51 |
| Comparative | H1 | 5.4 | 5.3 | (0.14, 0.05) | 30 |
| Example 1 | | | | | |
| Comparative | H2 | 5.3 | 5.4 | (0.14, 0.05) | 32 |
| Example 2 | | | | | |
| Comparative | H3 | 5.1 | 5.6 | (0.14, 0.05) | 31 |
| Example 3 | | | | | |
| Comparative | H4 | 5.1 | 5.7 | (0.14, 0.05) | 30 |
| Example 4 | | | | | |
| Comparative | H5 | 5.0 | 5.7 | (0.14, 0.05) | 34 |
| Example 5 | | | | | |
| Comparative | H6 | 5.0 | 5.6 | (0.14, 0.05) | 33 |
| Example 6 | | | | | |
| Comparative | H7 | 5.3 | 5.5 | (0.14, 0.05) | 30 |
| Example 7 | | | | | |

### Comparative Example Compound

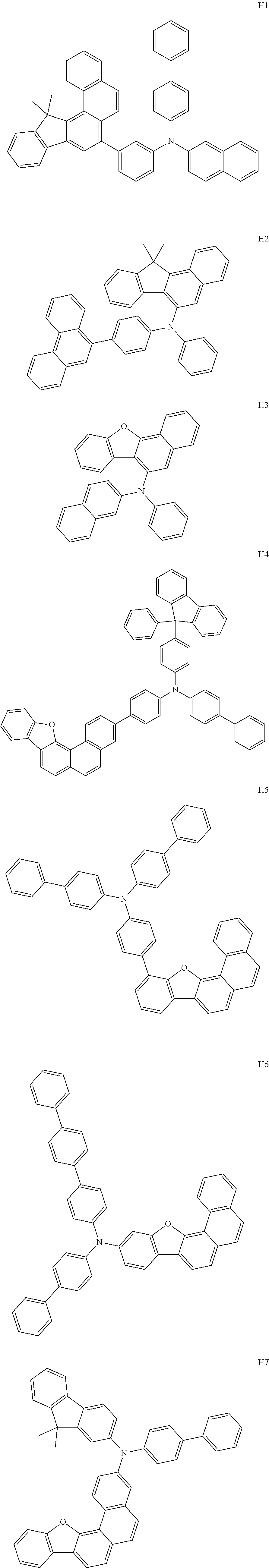

Compounds H1 to H7 are compounds having different basic skeletons or different substitution positions of the arylamine group from the heterocyclic compound represented by Chemical Formula 1. Comparative Examples 1 to 7 using Compounds H1 to H7 exhibited results of higher driving voltage, and lower light emission efficiency and lifetime properties compared to Examples 1 to 20.

From such results, it could be seen that the heterocyclic compound represented by Chemical Formula 1 of the present disclosure is capable of preventing device properties from declining by suppressing pi-pi stacking of the aromatic ring due to the substitution position of the arylamine group and thereby improving stability of the organic light emitting device.

Accordingly, it may be seen that, when the heterocyclic compound represented by Chemical Formula 1 of the present disclosure is used in a hole transport layer of a blue organic light emitting device, the organic light emitting device has low driving voltage, and excellent light emission efficiency and lifetime properties.

### Experimental Example 2

### Experimental Example 2-1. Manufacture of Organic Light Emitting Device

A transparent electrode ITO thin film obtained from glass for an OLED (manufactured by Samsung Corning Advanced Glass) was ultrasonic cleaned using trichloroethylene, acetone, ethanol and distilled water sequentially for 5 minutes each, and then stored in isopropanol before use.

The ITO substrate was installed in a substrate folder of a vacuum deposition apparatus, and 4,4′,4″-tris(N,N-(2-naphthyl)-phenylamino)triphenylamine (2-TNATA) was introduced thereto.

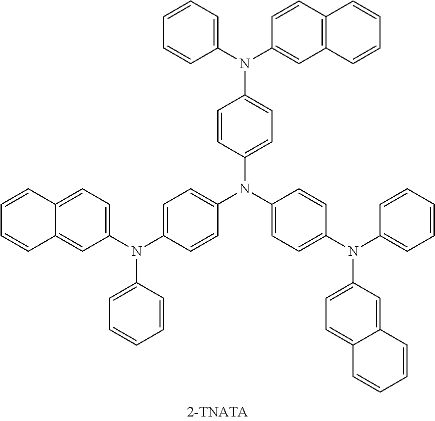 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, the chamber was evacuated until the degree of vacuum therein reached 106 torr, and then the 2-TNATA was evaporated by applying a current to the cell to deposit a hole injection layer having a thickness of 600 Å on the ITO substrate. To another cell in the vacuum deposition apparatus, N,N′-bis(α-naphthyl)-N,N′-diphenyl-4,4′-diamine (NPB) was introduced, and evaporated by applying a current to the cell to deposit a hole transport layer having a thickness of 300 Å on the hole injection layer.

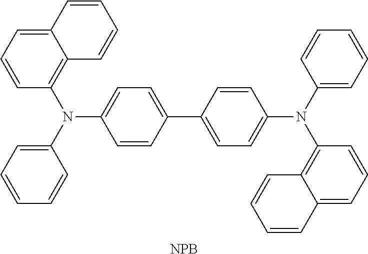 [SMILES: c1ccc(N(c2ccc(-c3ccc(N(c4ccccc4)c4cccc5ccccc45)cc3)cc2)c2cccc3ccccc23)cc1 ; "NPB"]

Subsequently, a compound represented by Chemical Formula 1 or a comparative compound described in Table 6 was deposited to a thickness of 50 Å as an electron blocking layer.

A blue light emitting material having the following structure was deposited thereon as a light emitting layer. Specifically, on one cell in the vacuum deposition apparatus, BH1 that is a blue light emitting host material was vacuum deposited to a thickness of 200 Å, and D1 that is a blue light emitting dopant material was vacuum deposited thereon by 5% with respect to the host material.

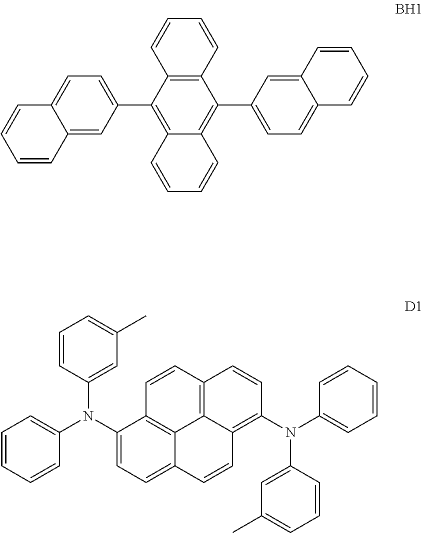

After that, a compound of the following structural formula E1 was deposited to a thickness of 300 Å as an electron transport layer.

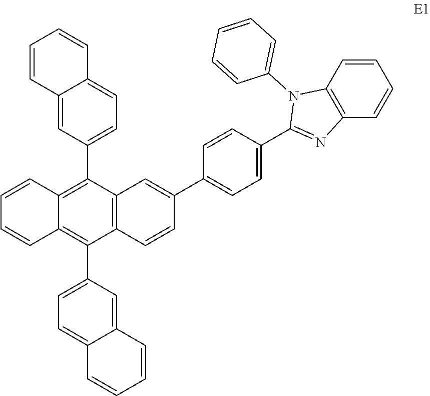 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

After that, lithium fluoride (LiF) was deposited to a thickness of 10 Å as an electron injection layer, and Al was deposited to a thickness of 1000 Å to form a negative electrode, and as a result, an organic light emitting device was manufactured.

Meanwhile, all the organic compounds required to manufacture the organic light emitting device were vacuum sublimation purified under 108 torr to 106 torr for each material to be used in the manufacture of the organic light emitting device.

### Experimental Example 2-2. Driving Voltage and Light Emission Efficiency of Organic Light Emitting Device

For each of the organic electroluminescent devices manufactured as above, electroluminescent (EL) properties were measured using M7000 manufactured by McScience Inc., and with the measurement results, a lifetime T95 that is a time taken for luminance to become 95% with respect to initial luminance was measured when standard luminance was 700 cd/m² through a lifetime measurement system (M6000) manufactured by McScience Inc.

Results of measuring driving voltage, light emission efficiency and lifetime (T95) of the blue organic light emitting device manufactured according to the manufacturing method are shown in Table 6.

TABLE 6

| | | | Light | | |
|---|---|---|---|---|---|
| | | Driving | Emission | | Life- |
| | | Voltage | Efficiency | CIE | time |
| | Compound | (V) | (cd/A) | (x, y) | (T95) |
| Example 21 | 2 | 4.4 | 6.5 | (0.14, 0.05) | 46 |
| Example 22 | 16 | 4.6 | 6.2 | (0.14, 0.05) | 42 |
| Example 23 | 27 | 4.9 | 5.9 | (0.14, 0.05) | 39 |
| Example 24 | 50 | 4.3 | 6.6 | (0.14, 0.05) | 46 |
| Example 25 | 71 | 4.7 | 6.1 | (0.14, 0.05) | 40 |
| Example 26 | 95 | 4.3 | 6.5 | (0.14, 0.05) | 48 |
| Example 27 | 117 | 4.7 | 6.0 | (0.14, 0.05) | 41 |
| Example 28 | 133 | 4.4 | 6.5 | (0.14, 0.05) | 48 |
| Example 29 | 144 | 4.4 | 6.7 | (0.14, 0.05) | 45 |
| Example 30 | 165 | 4.3 | 6.6 | (0.14, 0.05) | 48 |
| Example 31 | 189 | 4.3 | 6.7 | (0.14, 0.05) | 46 |
| Example 32 | 203 | 4.5 | 6.6 | (0.14, 0.05) | 47 |
| Example 33 | 226 | 4.8 | 6.1 | (0.14, 0.05) | 40 |
| Example 34 | 258 | 4.7 | 6.1 | (0.14, 0.05) | 40 |
| Example 35 | 262 | 4.3 | 6.5 | (0.14, 0.05) | 46 |
| Example 36 | 292 | 4.4 | 6.5 | (0.14, 0.05) | 47 |
| Example 37 | 314 | 4.2 | 6.7 | (0.14, 0.05) | 49 |
| Example 38 | 340 | 4.8 | 6.0 | (0.14, 0.05) | 41 |
| Example 39 | 343 | 4.4 | 6.6 | (0.14, 0.05) | 46 |
| Example 40 | 362 | 4.4 | 6.5 | (0.14, 0.05) | 50 |
| Comparative | H1 | 5.5 | 5.3 | (0.14, 0.05) | 29 |
| Example 8 | | | | | |
| Comparative | H2 | 5.4 | 5.3 | (0.14, 0.05) | 30 |
| Example 9 | | | | | |
| Comparative | H3 | 5.2 | 5.5 | (0.14, 0.05) | 32 |
| Example 10 | | | | | |
| Comparative | H4 | 5.2 | 5.6 | (0.14, 0.05) | 29 |
| Example 11 | | | | | |
| Comparative | H5 | 5.1 | 5.6 | (0.14, 0.05) | 35 |
| Example 12 | | | | | |
| Comparative | H6 | 5.0 | 5.5 | (0.14, 0.05) | 32 |
| Example 13 | | | | | |
| Comparative | H7 | 5.4 | 5.4 | (0.14, 0.05) | 30 |
| Example 14 | | | | | |

Compounds H1 to H7 are compounds having different basic skeletons or different substitution positions of the arylamine group from the heterocyclic compound represented by Chemical Formula 1. Comparative Examples 8 to 14 using Compounds H1 to H7 exhibited results of higher driving voltage, and lower light emission efficiency and lifetime properties compared to Examples 21 to 40.

When electrons migrate to a positive electrode after passing through a hole transport layer without being combined in a light emitting layer, light emission efficiency and lifetime of an organic light emitting device decrease. Using a compound having a high LUMO level in an electron blocking layer may prevent the above-described phenomenon since electrons migrating to a positive electrode after passing through a light emitting layer are blocked by an energy barrier of the electron blocking layer.

The heterocyclic compound represented by Chemical Formula 1 of the present disclosure has a high LUMO level. Accordingly, through the results of Table 6, it could be identified that, when the heterocyclic compound represented by Chemical Formula 1 of the present disclosure is used in an electron blocking layer of a blue organic light emitting device, a driving voltage of the organic light emitting device may be reduced, and light emission efficiency and lifetime properties thereof may be improved.

### Experimental Example 3

### Experimental Example 3-1. Manufacture of Organic Light Emitting Device

A glass substrate on which ITO was coated as a thin film to thicknesses of 1,500 Å was ultrasonic cleaned with distilled water. When the cleaning with distilled water was finished, the substrate was ultrasonic cleaned with solvents such as acetone, methanol and isopropyl alcohol, then dried, and then subjected to UVO (ultraviolet ozone) treatment for 5 minutes using UV (ultraviolet) in a UV cleaner. After that, the substrate was transferred to a plasma cleaner (PT), then subjected to plasma treatment under vacuum for ITO work function increase and residual film removal, and transferred to a thermal deposition apparatus for organic deposition.

On the transport ITO electrode (positive electrode), a hole injection layer 4,4′,4″-tris[2-naphthyl(phenyl)amino]triphenylamine (2-TNATA) and a hole transport layer N,N′-bis(α-naphthyl)-N,N′-diphenyl-4,4′-diamine (NPB) were deposited.

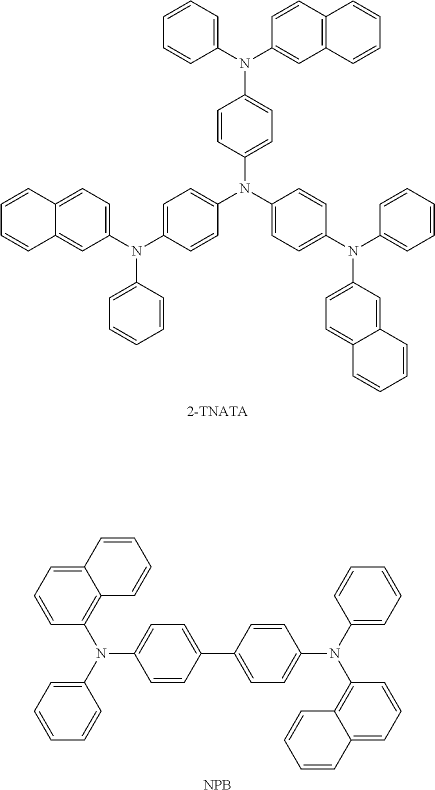 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1 ; c1ccc(N(c2ccc(-c3ccc(N(c4ccccc4)c4cccc5ccccc45)cc3)cc2)c2cccc3ccccc23)cc1 ; "NPB"]

A light emitting layer was thermal vacuum deposited thereon as follows. The light emitting layer was deposited to a thickness of 400 Å by pre-mixing one type of first host (heterocyclic compound represented by Chemical Formula 1, P type) and one type of second host (heterocyclic compound represented by Chemical Formula 2, N type) described in the following Table 7 and then depositing the mixture in one source of supply as a red host, using (Ir (piq) 2 (acac) as a red phosphorescent dopant, and by doping the red phosphorescent dopant to the host by 3%.

After that, Bphen was deposited to a thickness of 30 Å as a hole blocking layer, and TPBI was deposited to a thickness of 250 Å thereon as an electron transport layer. Lastly, lithium fluoride (LiF) was deposited on the electron transport layer to a thickness of 10 Å to form an electron injection layer, and then aluminum (Al) was deposited on the electron injection layer to a thickness of 1,200 Å to form a negative electrode, and as a result, an organic electroluminescent device was manufactured.

Meanwhile, all the organic compounds required to manufacture the OLED were vacuum sublimation purified under 108 torr to 106 torr for each material to be used in the manufacture of the OLED (organic light emitting device).

### Experimental Example 3-2. Driving Voltage and Light Emission Efficiency of Organic Light Emitting Device

For each of the organic electroluminescent devices manufactured as above, electroluminescent (EL) properties were measured using M7000 manufactured by McScience Inc., and with the measurement results, a lifetime T95 that is a time taken for luminance to become 95% with respect to initial luminance was measured when standard luminance was 700 cd/m² through a lifetime measurement system (M6000) manufactured by McScience Inc.

Results of measuring driving voltage, light emission efficiency and lifetime (T9s) of the red organic light emitting device manufactured according to the manufacturing method are shown in Table 7.

TABLE 7

| | | | | | Light | | |
|---|---|---|---|---|---|---|---|
| | | | | Driving | Emission | | |
| | Compound | | Ratio | Voltage | Efficiency | CIE | Lifetime |
| | P | N | (P:N) | (V) | (cd/A) | (x, y) | (T95) |
| Example 41 | 2 | NH5 | 1:3 | 4.4 | 54.1 | (0.68, 0.32) | 180 |
| Example 42 | 2 | NH5 | 1:2 | 4.3 | 55.3 | (0.68, 0.32) | 189 |
| Example 43 | 2 | NH5 | 1:1 | 4.2 | 57.1 | (0.68, 0.32) | 201 |
| Example 44 | 2 | NH5 | 2:1 | 4.3 | 55.4 | (0.68, 0.32) | 176 |
| Example 45 | 2 | NH5 | 3:1 | 4.4 | 54.9 | (0.68, 0.32) | 162 |
| Example 46 | 16 | NH30 | 1:1 | 4.4 | 54.2 | (0.68, 0.32) | 175 |
| Example 47 | 27 | NH30 | 1:1 | 4.7 | 51.8 | (0.68, 0.32) | 158 |
| Example 48 | 50 | NH30 | 1:1 | 4.2 | 57.4 | (0.68, 0.32) | 215 |
| Example 49 | 71 | NH79 | 1:1 | 4.4 | 52.4 | (0.68, 0.32) | 160 |
| Example 50 | 95 | NH79 | 1:1 | 4.2 | 58.0 | (0.68, 0.32) | 226 |
| Example 51 | 117 | NH79 | 1:1 | 4.4 | 53.5 | (0.68, 0.32) | 173 |
| Example 52 | 133 | NH161 | 1:1 | 4.2 | 57.4 | (0.68, 0.32) | 206 |
| Example 53 | 144 | NH161 | 1:1 | 4.1 | 58.6 | (0.68, 0.32) | 219 |
| Example 54 | 165 | NH161 | 1:1 | 4.1 | 58.3 | (0.68, 0.32) | 224 |
| Example 55 | 189 | NH147 | 1:1 | 4.2 | 57.7 | (0.68, 0.32) | 218 |
| Example 56 | 203 | NH147 | 1:1 | 4.2 | 57.3 | (0.68, 0.32) | 205 |
| Example 57 | 226 | NH84 | 1:1 | 4.5 | 52.8 | (0.68, 0.32) | 170 |
| Example 58 | 258 | NH84 | 1:1 | 4.4 | 53.1 | (0.68, 0.32) | 161 |
| Example 59 | 262 | NH43 | 1:1 | 4.1 | 57.0 | (0.68, 0.32) | 203 |
| Example 60 | 292 | NH43 | 1:1 | 4.2 | 58.4 | (0.68, 0.32) | 227 |
| Example 61 | 314 | NH128 | 1:1 | 4.2 | 58.8 | (0.68, 0.32) | 213 |
| Example 62 | 340 | NH128 | 1:1 | 4.5 | 53.2 | (0.68, 0.32) | 175 |
| Example 63 | 343 | NH5 | 1:1 | 4.1 | 57.5 | (0.68, 0.32) | 207 |
| Example 64 | 362 | NH5 | 1:1 | 4.1 | 58.5 | (0.68, 0.32) | 234 |
| Comparative | H1 | NH30 | 1:1 | 4.9 | 46.1 | (0.68, 0.32) | 100 |
| Example 15 | | | | | | | |
| Comparative | H2 | NH79 | 1:1 | 4.9 | 45.4 | (0.68, 0.32) | 113 |
| Example 16 | | | | | | | |
| Comparative | H3 | NH161 | 1:1 | 4.8 | 47.7 | (0.68, 0.32) | 105 |
| Example 17 | | | | | | | |
| Comparative | H4 | NH147 | 1:1 | 4.8 | 46.3 | (0.68, 0.32) | 121 |
| Example 18 | | | | | | | |
| Comparative | H5 | NH84 | 1:1 | 4.7 | 48.5 | (0.68, 0.32) | 108 |
| Example 19 | | | | | | | |
| Comparative | H6 | NH43 | 1:1 | 4.9 | 46.8 | (0.68,0.32) | 119 |
| Example 20 | | | | | | | |
| Comparative | H7 | NH128 | 1:1 | 4.9 | 46.6 | (0.68, 0.32) | 104 |
| Example 21 | | | | | | | |

From the results of Table 7, it may be identified that driving voltage, light emission efficiency and lifetime are all improved when the heterocyclic compound represented by Chemical Formula 1 and the heterocyclic compound represented by Chemical Formula 2 of the present disclosure are used at the same time as a host of a light emitting layer.

In other words, it was able to be identified that excellent device properties were obtained when, as a host of a light emitting layer, the heterocyclic compound represented by Chemical Formula 1 was used for a donor role and the heterocyclic compound represented by Chemical Formula 2 was used for an acceptor role in the present disclosure.

On the other hand, it may be seen that, when the Comparative Example Compound and the heterocyclic compound represented by Chemical Formula 2 are used at the same time, performance in terms of driving voltage, light emission efficiency and lifetime declines compared to in the Examples.

Accordingly, it was able to be identified that driving voltage was low, and light emission efficiency and lifetime were significantly superior when the heterocyclic compound represented by Chemical Formula 1 and the heterocyclic compound represented by Chemical Formula 2 of the present disclosure are used at the same time as a host of a light emitting layer.

### REFERENCE NUMERAL

100: Substrate
200: Positive Electrode
300: Organic Material Layer
301: Hole Injection Layer
302: Hole Transport Layer
303: Light Emitting Layer
304: Hole Blocking Layer
305: Electron Transport Layer
306: Electron Injection Layer
400: Negative Electrode

What is claimed is:

1. A heterocyclic compound represented by the following Chemical Formula 1: 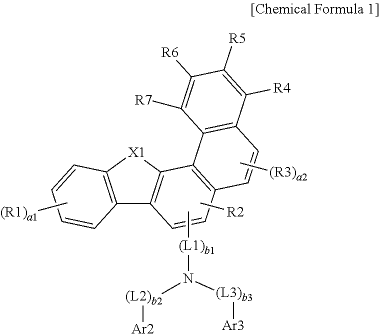
wherein, in Chemical Formula 1,
X1 is O; S; or NAr1;
R1 to R7 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; and —NR101R102, or two or more groups adjacent to each other bond to each other to form a substituted or unsubstituted C6 to C60 aromatic hydrocarbon ring; or a substituted or unsubstituted C2 to C60 heteroring, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group;
a1 is an integer of 0 to 4, and when a1 is 2 or greater, R1s are the same as or different from each other;
a2 is an integer of 0 to 2, and when a2 is 2 or greater, R3s are the same as or different from each other;
Ar1 is a substituted or unsubstituted C6 to C60 aryl group; or a substituted or unsubstituted C2 to C60 heteroaryl group;
Ar2 and Ar3 are the same as or different from each other, and each independently a substituted or unsubstituted C6 to C60 aryl group; a substituted or unsubstituted C2 to C60 heteroaryl group; or —NAr4Ar5;
Ar4 and Ar5 are the same as or different from each other, and each independently a substituted or unsubstituted C6 to C60 aryl group; or a substituted or unsubstituted C2 to C60 heteroaryl group;
L1 to L3 are the same as or different from each other, and each independently a direct bond; a substituted or unsubstituted C6 to C60 arylene group; or a substituted or unsubstituted C2 to C60 heteroarylene group;
b1 is an integer of 0 to 5, and when b1 is 2 or greater, L1s are the same as or different from each other;
b2 is an integer of 0 to 5, and when b2 is 2 or greater, L2s are the same as or different from each other; and
b3 is an integer of 0 to 5, and when b3 is 2 or greater, L3s are the same as or different from each other.

2. The heterocyclic compound of claim 1, wherein the heterocyclic compound represented by Chemical Formula 1 is represented by any one of the following Chemical Formulae 1-1 and 1-2: 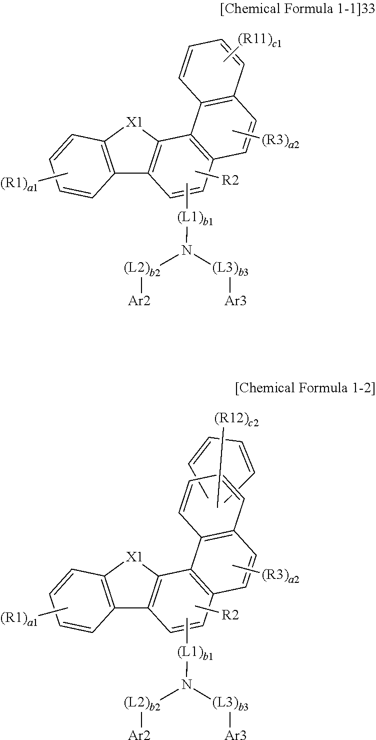
in Chemical Formulae 1-1 and 1-2,
R11 and R12 are the same as or different from each other and each independently hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; a substituted or unsubstituted C6 to C60 aryl group; a substituted or unsubstituted C2 to C60 heteroaryl group; —P(═O)R101R102; —SiR101R102R103; or —NR101R102, and R101, R102 and R103 are the same as or different from each other and each independently a substituted or unsubstituted C1 to C60 alkyl group; a substituted or unsubstituted C6 to C60 aryl group; or a substituted or unsubstituted C2 to C60 heteroaryl group;
c1 is an integer of 0 to 4, and when c1 is 2 or greater, R11s are the same as or different from each other;
c2 is an integer of 0 to 6, and when c2 is 2 or greater, R12s are the same as or different from each other; and
X1, R1 to R3, L1 to L3, Ar2, Ar3, a1, a2, and b1 to b3 have the same definitions as in Chemical Formula 1.

3. The heterocyclic compound of claim 2, wherein the heterocyclic compound represented by Chemical Formula 1-2 is represented by any one of the following Chemical Formulae 1-2-1 to 1-2-3: 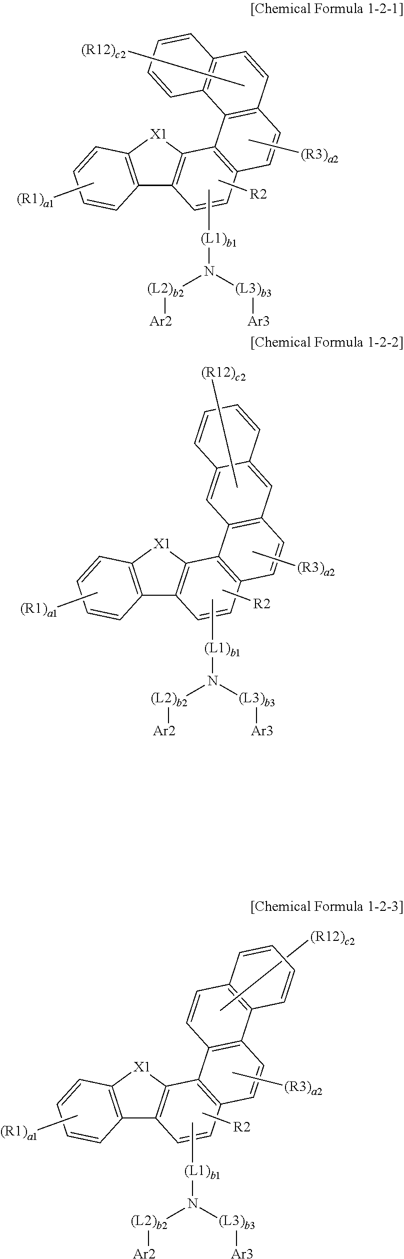
in Chemical Formulae 1-2-1 to 1-2-3,
R12 and c2 have the same definitions as in Chemical Formula 1-2; and
X1, R1 to R3, L1 to L3, Ar2, Ar3, a1, a2, and b1 to b3 have the same definitions as in Chemical Formula 1.

4. The heterocyclic compound of claim 1, wherein R1 to R3 are the same as or different from each other, and each independently hydrogen; or deuterium.

5. The heterocyclic compound of claim 1, wherein the heterocyclic compound represented by Chemical Formula 1 does not include deuterium as a substituent, or has a deuterium content of 1% to 100% with respect to a total number of hydrogen atoms and deuterium atoms.

6. The heterocyclic compound of claim 1, wherein the heterocyclic compound represented by Chemical Formula 1 is represented by any one of the following compounds:

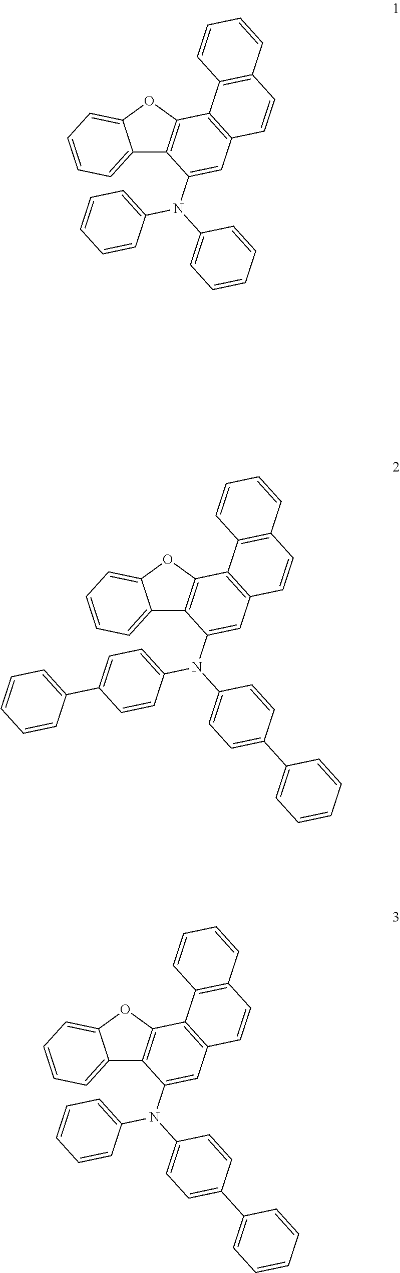 [SMILES: c1ccc(N(c2ccccc2)c2cc3ccc4ccccc4c3c3oc4ccccc4c23)cc1 ; c1ccc(-c2ccc(N(c3ccc(-c4ccccc4)cc3)c3cc4ccc5ccccc5c4c4oc5ccccc5c34)cc2)cc1 ; c1ccc(-c2ccc(N(c3ccccc3)c3cc4ccc5ccccc5c4c4oc5ccccc5c34)cc2)cc1]

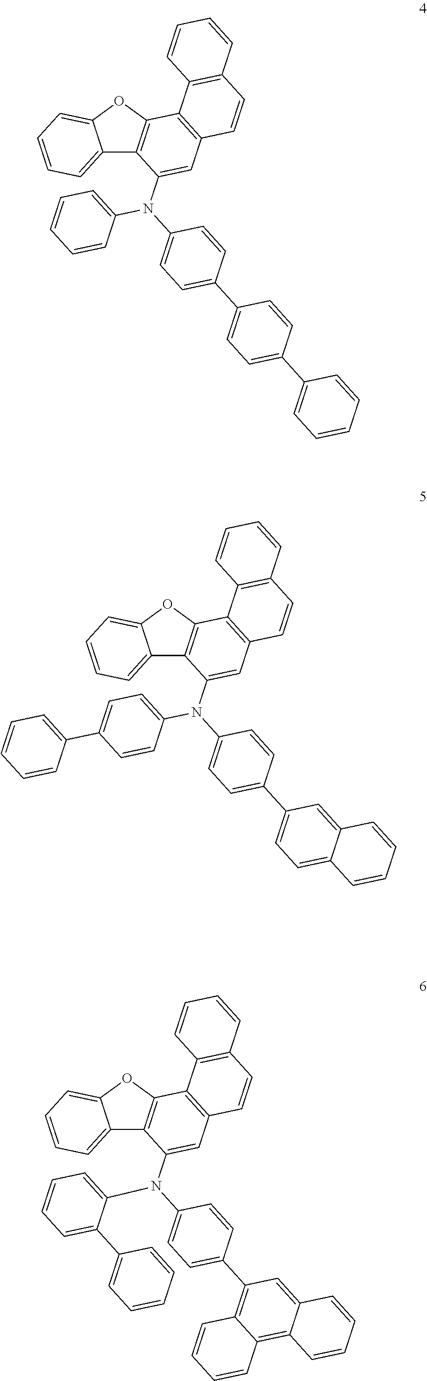 [SMILES: c1ccc(-c2ccc(-c3ccc(N(c4ccccc4)c4cc5ccc6ccccc6c5c5oc6ccccc6c45)cc3)cc2)cc1 ; c1ccc(-c2ccc(N(c3ccc(-c4ccc5ccccc5c4)cc3)c3cc4ccc5ccccc5c4c4oc5ccccc5c34)cc2)cc1 ; c1ccc(-c2ccccc2N(c2ccc(-c3cc4ccccc4c4ccccc34)cc2)c2cc3ccc4ccccc4c3c3oc4ccccc4c23)cc1]

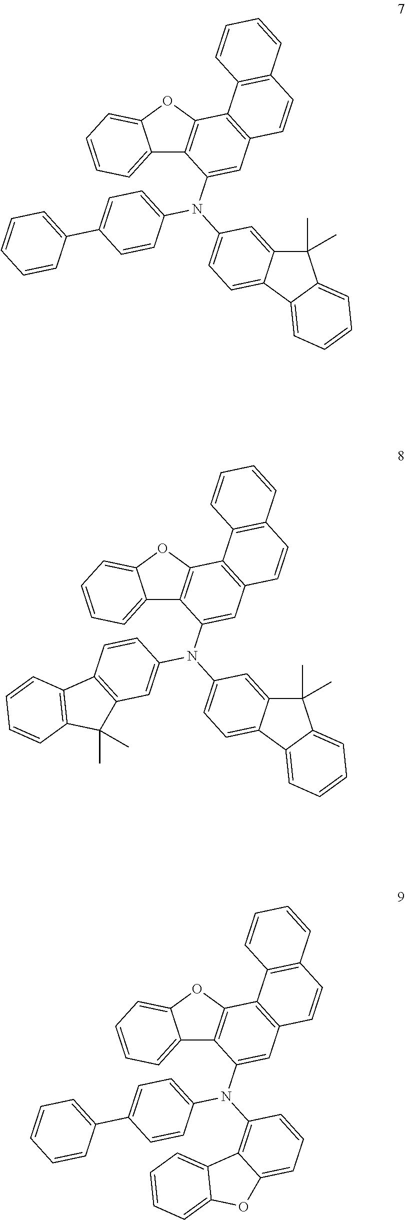 [SMILES: CC1(C)c2ccccc2-c2ccc(N(c3ccc(-c4ccccc4)cc3)c3cc4ccc5ccccc5c4c4oc5ccccc5c34)cc21 ; CC1(C)c2ccccc2-c2ccc(N(c3ccc4c(c3)C(C)(C)c3ccccc3-4)c3cc4ccc5ccccc5c4c4oc5ccccc5c34)cc21 ; c1ccc(-c2ccc(N(c3cccc4oc5ccccc5c34)c3cc4ccc5ccccc5c4c4oc5ccccc5c34)cc2)cc1]

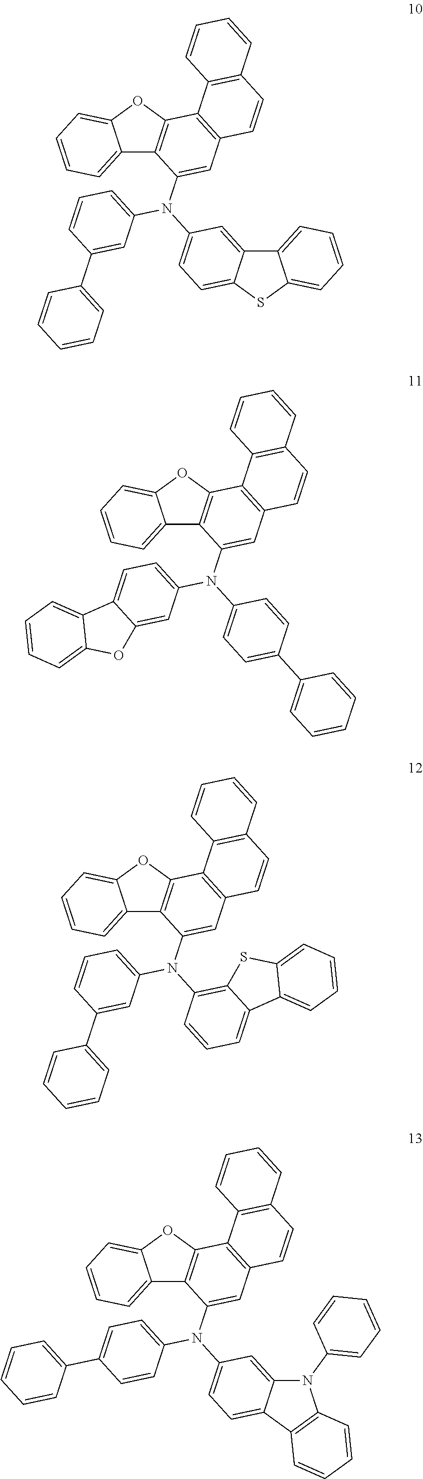 [SMILES: c1ccc(-c2cccc(N(c3ccc4sc5ccccc5c4c3)c3cc4ccc5ccccc5c4c4oc5ccccc5c34)c2)cc1 ; c1ccc(-c2ccc(N(c3ccc4c(c3)oc3ccccc34)c3cc4ccc5ccccc5c4c4oc5ccccc5c34)cc2)cc1 ; c1ccc(-c2cccc(N(c3cccc4c3sc3ccccc34)c3cc4ccc5ccccc5c4c4oc5ccccc5c34)c2)cc1 ; c1ccc(-c2ccc(N(c3ccc4c5ccccc5n(-c5ccccc5)c4c3)c3cc4ccc5ccccc5c4c4oc5ccccc5c34)cc2)cc1]

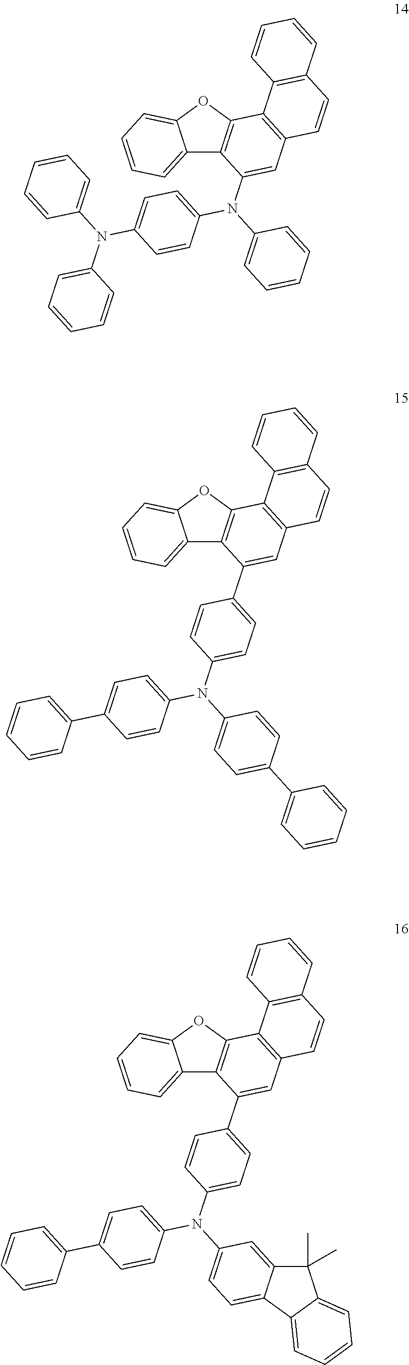 [SMILES: c1ccc(N(c2ccccc2)c2ccc(N(c3ccccc3)c3cc4ccc5ccccc5c4c4oc5ccccc5c34)cc2)cc1 ; c1ccc(-c2ccc(N(c3ccc(-c4ccccc4)cc3)c3ccc(-c4cc5ccc6ccccc6c5c5oc6ccccc6c45)cc3)cc2)cc1 ; CC1(C)c2ccccc2-c2ccc(N(c3ccc(-c4ccccc4)cc3)c3ccc(-c4cc5ccc6ccccc6c5c5oc6ccccc6c45)cc3)cc21]

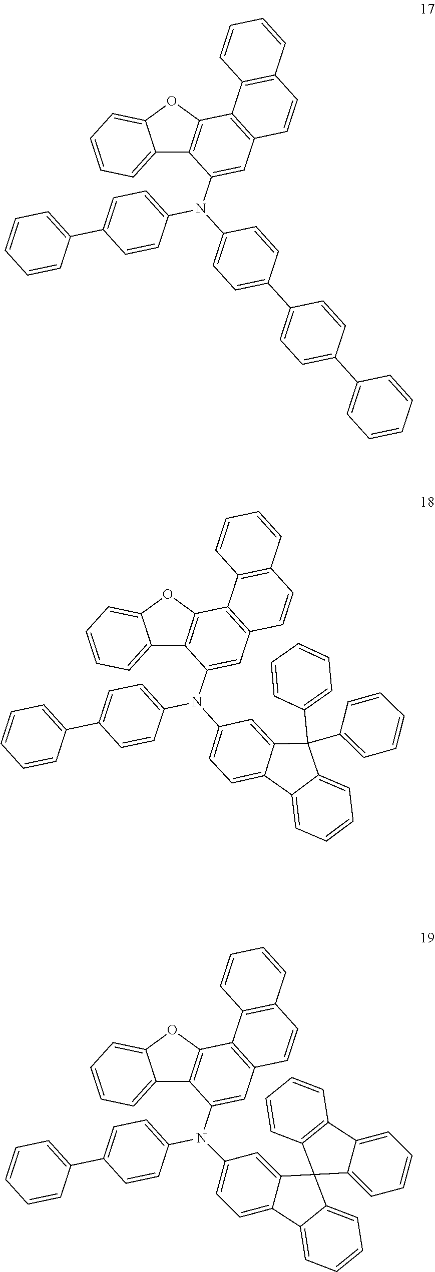 [SMILES: c1ccc(-c2ccc(-c3ccc(N(c4ccc(-c5ccccc5)cc4)c4cc5ccc6ccccc6c5c5oc6ccccc6c45)cc3)cc2)cc1 ; c1ccc(-c2ccc(N(c3ccc4c(c3)C(c3ccccc3)(c3ccccc3)c3ccccc3-4)c3cc4ccc5ccccc5c4c4oc5ccccc5c34)cc2)cc1 ; c1ccc(-c2ccc(N(c3ccc4c(c3)C3(c5ccccc5-c5ccccc53)c3ccccc3-4)c3cc4ccc5ccccc5c4c4oc5ccccc5c34)cc2)cc1]

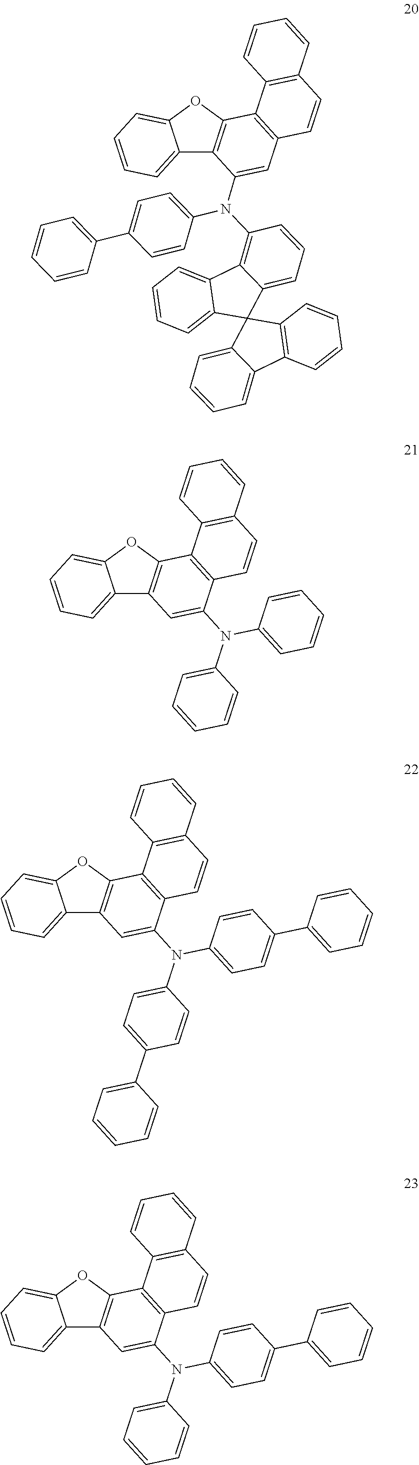 [SMILES: c1ccc(-c2ccc(N(c3cccc4c3-c3ccccc3C43c4ccccc4-c4ccccc43)c3cc4ccc5ccccc5c4c4oc5ccccc5c34)cc2)cc1 ; c1ccc(N(c2ccccc2)c2cc3c4ccccc4oc3c3c2ccc2ccccc23)cc1 ; c1ccc(-c2ccc(N(c3ccc(-c4ccccc4)cc3)c3cc4c5ccccc5oc4c4c3ccc3ccccc34)cc2)cc1 ; c1ccc(-c2ccc(N(c3ccccc3)c3cc4c5ccccc5oc4c4c3ccc3ccccc34)cc2)cc1]

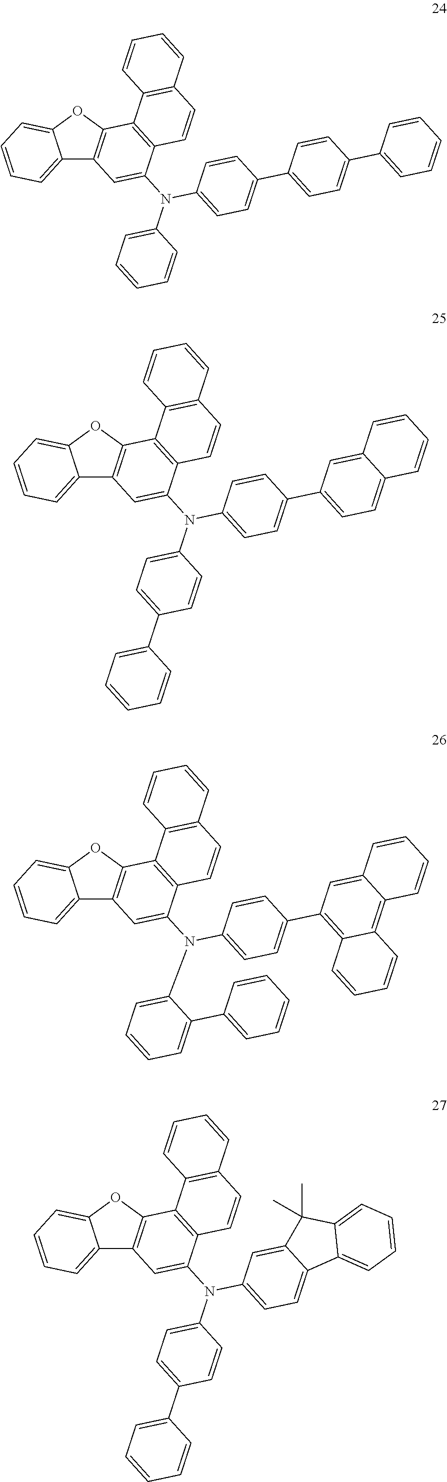 [SMILES: c1ccc(-c2ccc(-c3ccc(N(c4ccccc4)c4cc5c6ccccc6oc5c5c4ccc4ccccc45)cc3)cc2)cc1 ; c1ccc(-c2ccc(N(c3ccc(-c4ccc5ccccc5c4)cc3)c3cc4c5ccccc5oc4c4c3ccc3ccccc34)cc2)cc1 ; c1ccc(-c2ccccc2N(c2ccc(-c3cc4ccccc4c4ccccc34)cc2)c2cc3c4ccccc4oc3c3c2ccc2ccccc23)cc1 ; CC1(C)c2ccccc2-c2ccc(N(c3ccc(-c4ccccc4)cc3)c3cc4c5ccccc5oc4c4c3ccc3ccccc34)cc21]

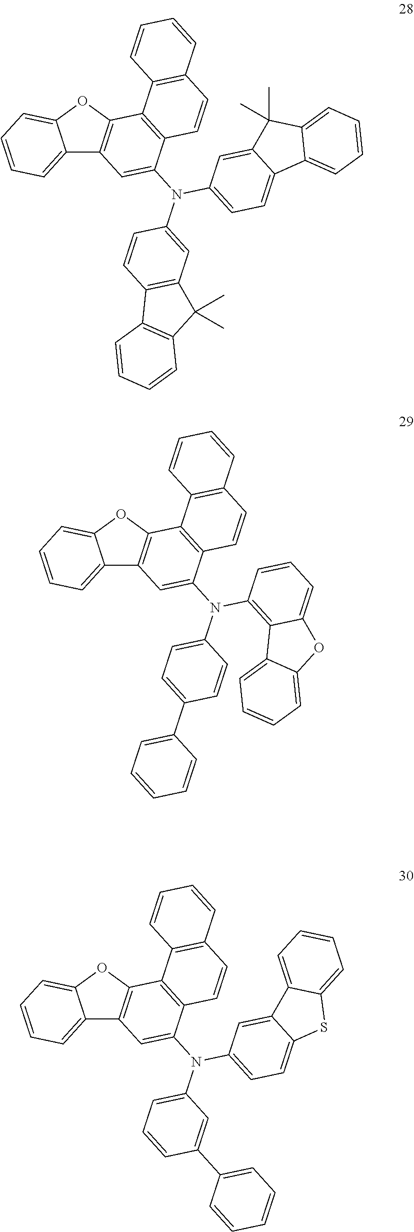 [SMILES: CC1(C)c2ccccc2-c2ccc(N(c3ccc4c(c3)C(C)(C)c3ccccc3-4)c3cc4c5ccccc5oc4c4c3ccc3ccccc34)cc21 ; c1ccc(-c2ccc(N(c3cc4c5ccccc5oc4c4c3ccc3ccccc34)c3cccc4oc5ccccc5c34)cc2)cc1 ; c1ccc(-c2cccc(N(c3ccc4sc5ccccc5c4c3)c3cc4c5ccccc5oc4c4c3ccc3ccccc34)c2)cc1]

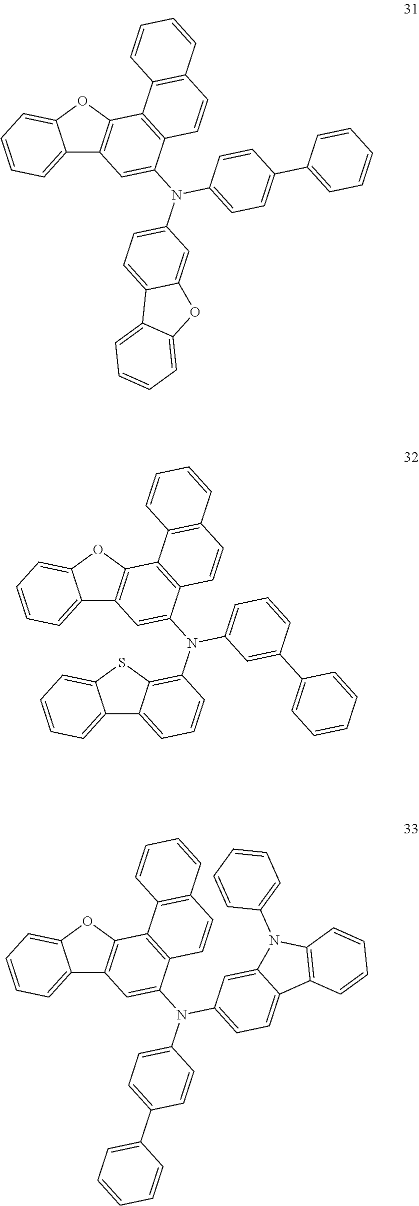 [SMILES: c1ccc(-c2ccc(N(c3ccc4c(c3)oc3ccccc34)c3cc4c5ccccc5oc4c4c3ccc3ccccc34)cc2)cc1 ; c1ccc(-c2cccc(N(c3cc4c5ccccc5oc4c4c3ccc3ccccc34)c3cccc4c3sc3ccccc34)c2)cc1 ; c1ccc(-c2ccc(N(c3ccc4c5ccccc5n(-c5ccccc5)c4c3)c3cc4c5ccccc5oc4c4c3ccc3ccccc34)cc2)cc1]

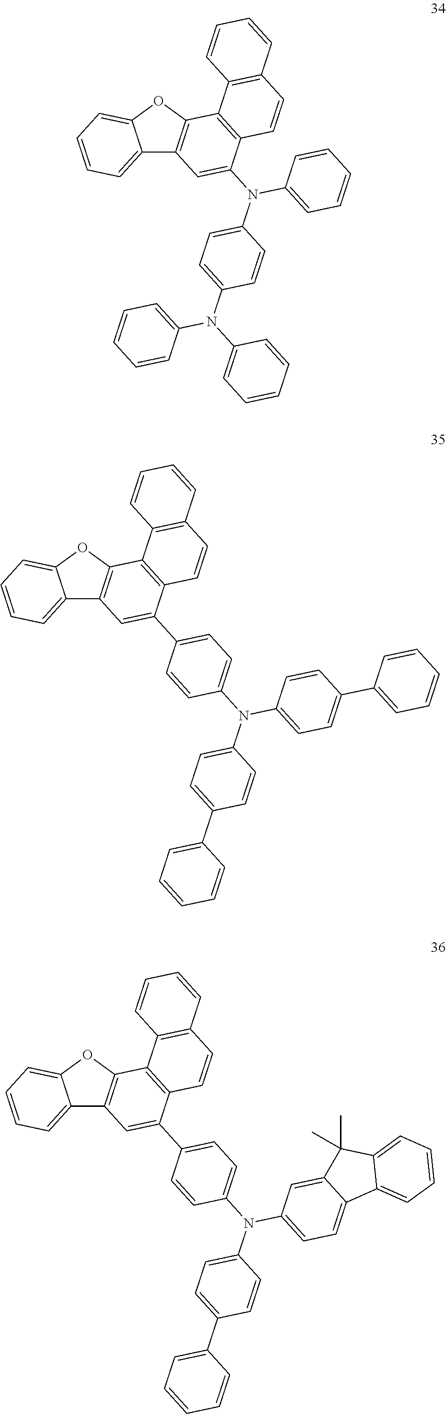 [SMILES: c1ccc(N(c2ccccc2)c2ccc(N(c3ccccc3)c3cc4c5ccccc5oc4c4c3ccc3ccccc34)cc2)cc1 ; c1ccc(-c2ccc(N(c3ccc(-c4ccccc4)cc3)c3ccc(-c4cc5c6ccccc6oc5c5c4ccc4ccccc45)cc3)cc2)cc1 ; CC1(C)c2ccccc2-c2ccc(N(c3ccc(-c4ccccc4)cc3)c3ccc(-c4cc5c6ccccc6oc5c5c4ccc4ccccc45)cc3)cc21]

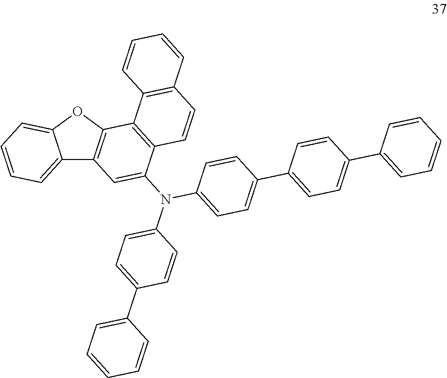 [SMILES: c1ccc(-c2ccc(-c3ccc(N(c4ccc(-c5ccccc5)cc4)c4cc5c6ccccc6oc5c5c4ccc4ccccc45)cc3)cc2)cc1]

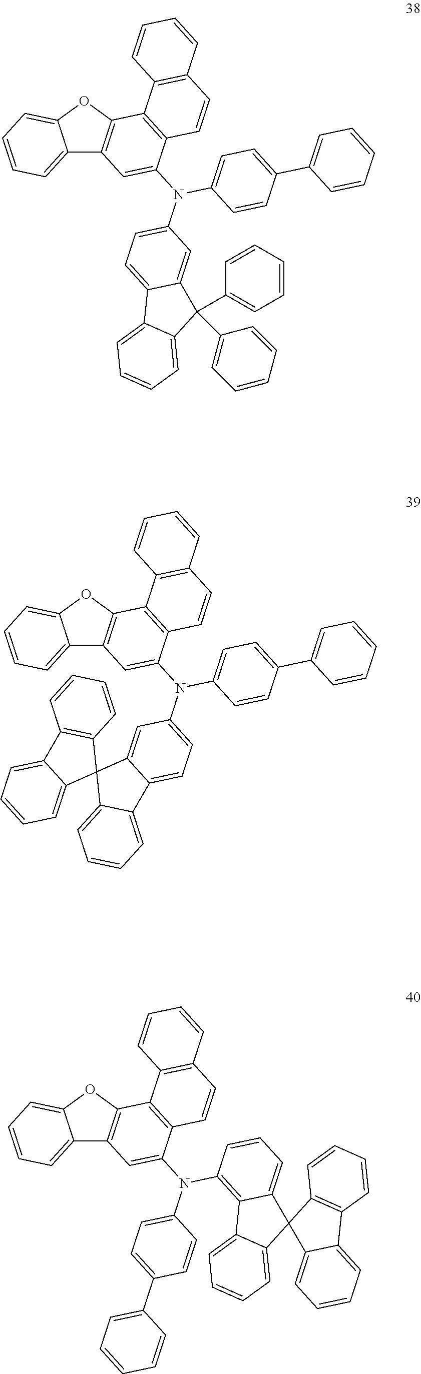 [SMILES: c1ccc(-c2ccc(N(c3ccc4c(c3)C(c3ccccc3)(c3ccccc3)c3ccccc3-4)c3cc4c5ccccc5oc4c4c3ccc3ccccc34)cc2)cc1 ; c1ccc(-c2ccc(N(c3ccc4c(c3)C3(c5ccccc5-c5ccccc53)c3ccccc3-4)c3cc4c5ccccc5oc4c4c3ccc3ccccc34)cc2)cc1 ; c1ccc(-c2ccc(N(c3cccc4c3-c3ccccc3C43c4ccccc4-c4ccccc43)c3cc4c5ccccc5oc4c4c3ccc3ccccc34)cc2)cc1]

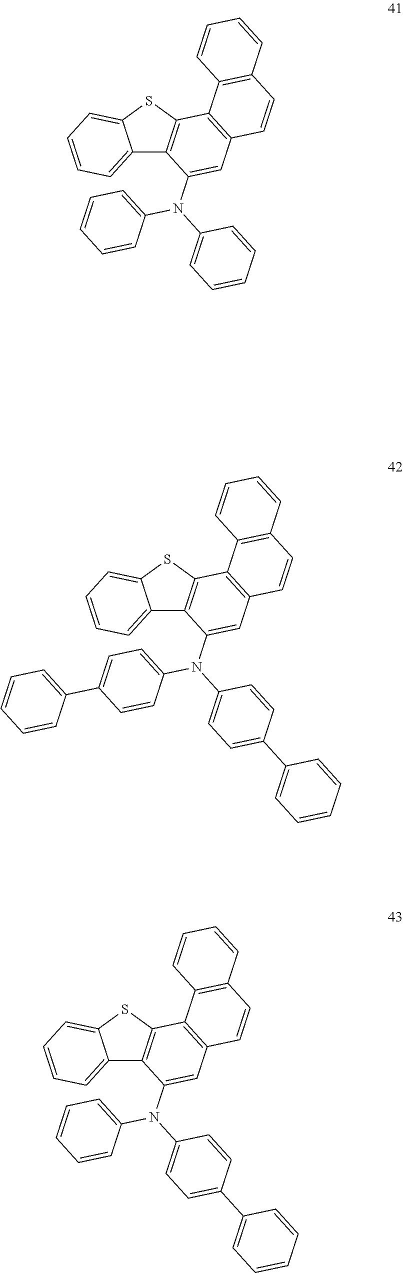 [SMILES: c1ccc(N(c2ccccc2)c2cc3ccc4ccccc4c3c3sc4ccccc4c23)cc1 ; c1ccc(-c2ccc(N(c3ccc(-c4ccccc4)cc3)c3cc4ccc5ccccc5c4c4sc5ccccc5c34)cc2)cc1 ; c1ccc(-c2ccc(N(c3ccccc3)c3cc4ccc5ccccc5c4c4sc5ccccc5c34)cc2)cc1]

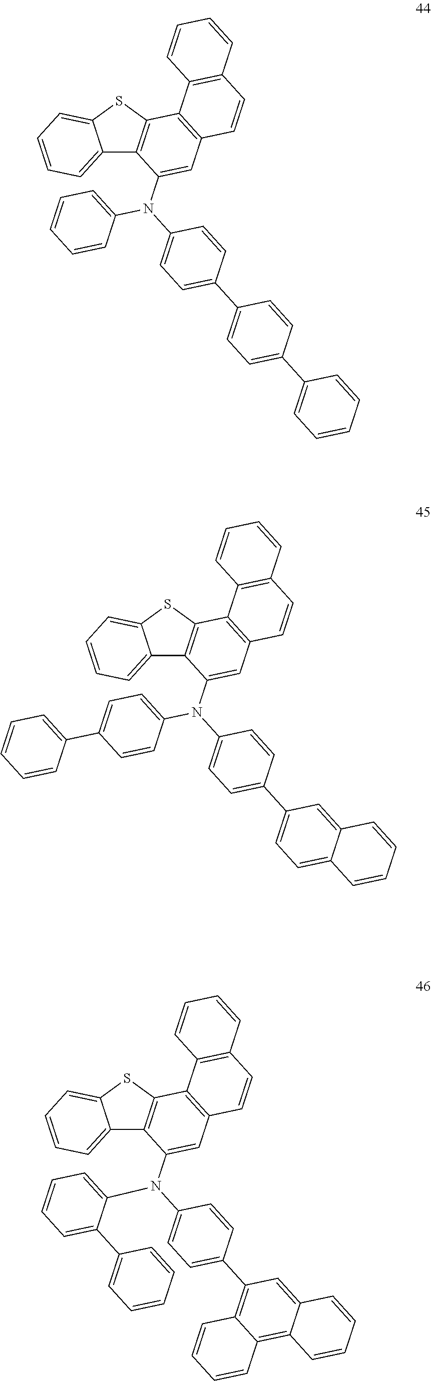 [SMILES: c1ccc(-c2ccc(-c3ccc(N(c4ccccc4)c4cc5ccc6ccccc6c5c5sc6ccccc6c45)cc3)cc2)cc1 ; c1ccc(-c2ccc(N(c3ccc(-c4ccc5ccccc5c4)cc3)c3cc4ccc5ccccc5c4c4sc5ccccc5c34)cc2)cc1 ; c1ccc(-c2ccccc2N(c2ccc(-c3cc4ccccc4c4ccccc34)cc2)c2cc3ccc4ccccc4c3c3sc4ccccc4c23)cc1]

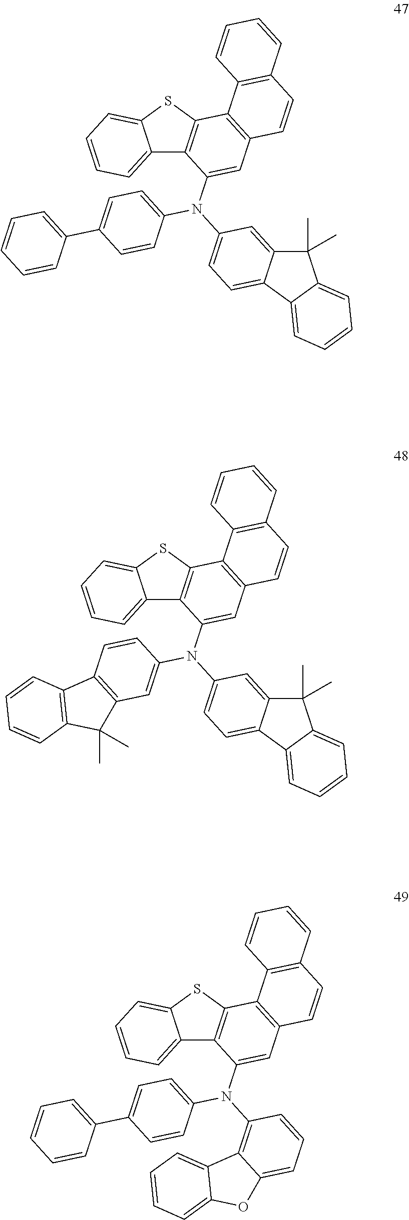 [SMILES: CC1(C)c2ccccc2-c2ccc(N(c3ccc(-c4ccccc4)cc3)c3cc4ccc5ccccc5c4c4sc5ccccc5c34)cc21 ; CC1(C)c2ccccc2-c2ccc(N(c3ccc4c(c3)C(C)(C)c3ccccc3-4)c3cc4ccc5ccccc5c4c4sc5ccccc5c34)cc21 ; c1ccc(-c2ccc(N(c3cccc4oc5ccccc5c34)c3cc4ccc5ccccc5c4c4sc5ccccc5c34)cc2)cc1]

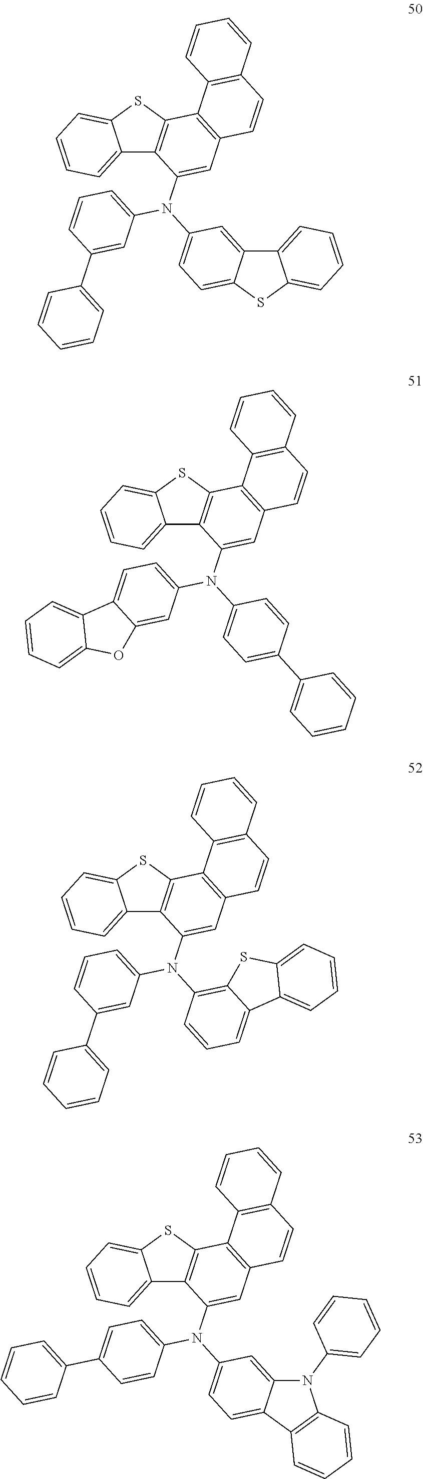 [SMILES: c1ccc(-c2cccc(N(c3ccc4sc5ccccc5c4c3)c3cc4ccc5ccccc5c4c4sc5ccccc5c34)c2)cc1 ; c1ccc(-c2ccc(N(c3ccc4c(c3)oc3ccccc34)c3cc4ccc5ccccc5c4c4sc5ccccc5c34)cc2)cc1 ; c1ccc(-c2cccc(N(c3cccc4c3sc3ccccc34)c3cc4ccc5ccccc5c4c4sc5ccccc5c34)c2)cc1 ; c1ccc(-c2ccc(N(c3ccc4c5ccccc5n(-c5ccccc5)c4c3)c3cc4ccc5ccccc5c4c4sc5ccccc5c34)cc2)cc1]

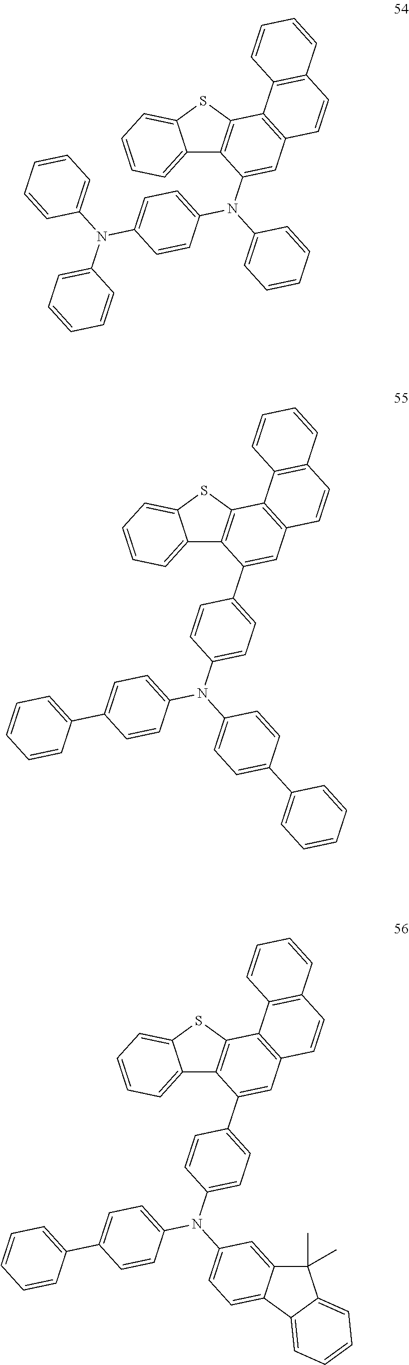 [SMILES: c1ccc(N(c2ccccc2)c2ccc(N(c3ccccc3)c3cc4ccc5ccccc5c4c4sc5ccccc5c34)cc2)cc1 ; c1ccc(-c2ccc(N(c3ccc(-c4ccccc4)cc3)c3ccc(-c4cc5ccc6ccccc6c5c5sc6ccccc6c45)cc3)cc2)cc1 ; CC1(C)c2ccccc2-c2ccc(N(c3ccc(-c4ccccc4)cc3)c3ccc(-c4cc5ccc6ccccc6c5c5sc6ccccc6c45)cc3)cc21]

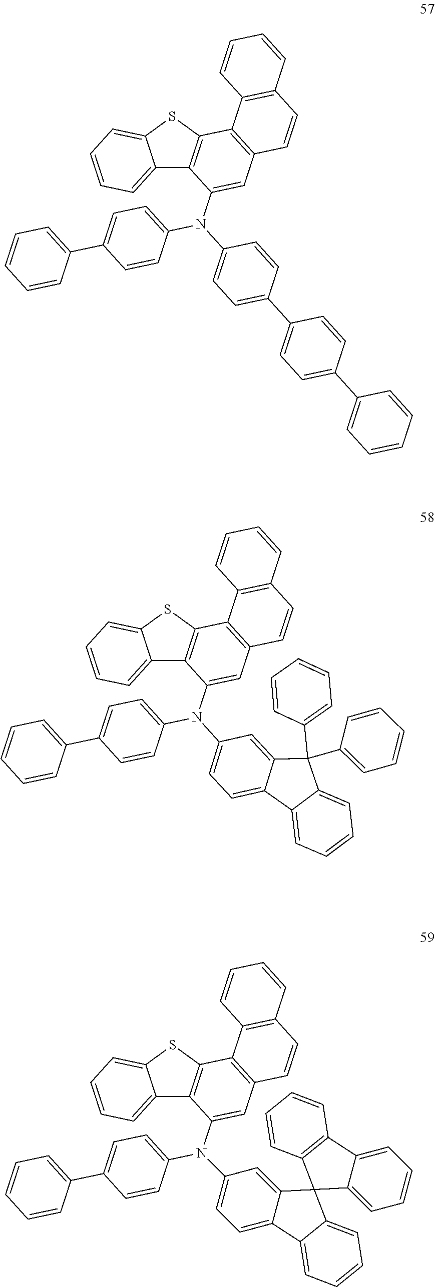 [SMILES: c1ccc(-c2ccc(-c3ccc(N(c4ccc(-c5ccccc5)cc4)c4cc5ccc6ccccc6c5c5sc6ccccc6c45)cc3)cc2)cc1 ; c1ccc(-c2ccc(N(c3ccc4c(c3)C(c3ccccc3)(c3ccccc3)c3ccccc3-4)c3cc4ccc5ccccc5c4c4sc5ccccc5c34)cc2)cc1 ; c1ccc(-c2ccc(N(c3ccc4c(c3)C3(c5ccccc5-c5ccccc53)c3ccccc3-4)c3cc4ccc5ccccc5c4c4sc5ccccc5c34)cc2)cc1]

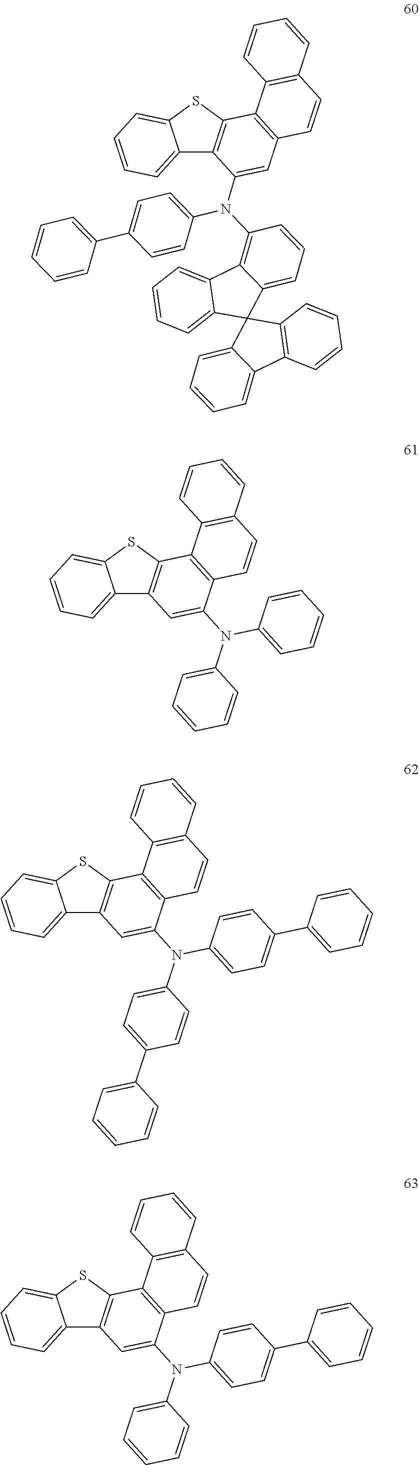 [SMILES: c1ccc(-c2ccc(N(c3cccc4c3-c3ccccc3C43c4ccccc4-c4ccccc43)c3cc4ccc5ccccc5c4c4sc5ccccc5c34)cc2)cc1 ; c1ccc(N(c2ccccc2)c2cc3c4ccccc4sc3c3c2ccc2ccccc23)cc1 ; c1ccc(-c2ccc(N(c3ccc(-c4ccccc4)cc3)c3cc4c5ccccc5sc4c4c3ccc3ccccc34)cc2)cc1 ; c1ccc(-c2ccc(N(c3ccccc3)c3cc4c5ccccc5sc4c4c3ccc3ccccc34)cc2)cc1]

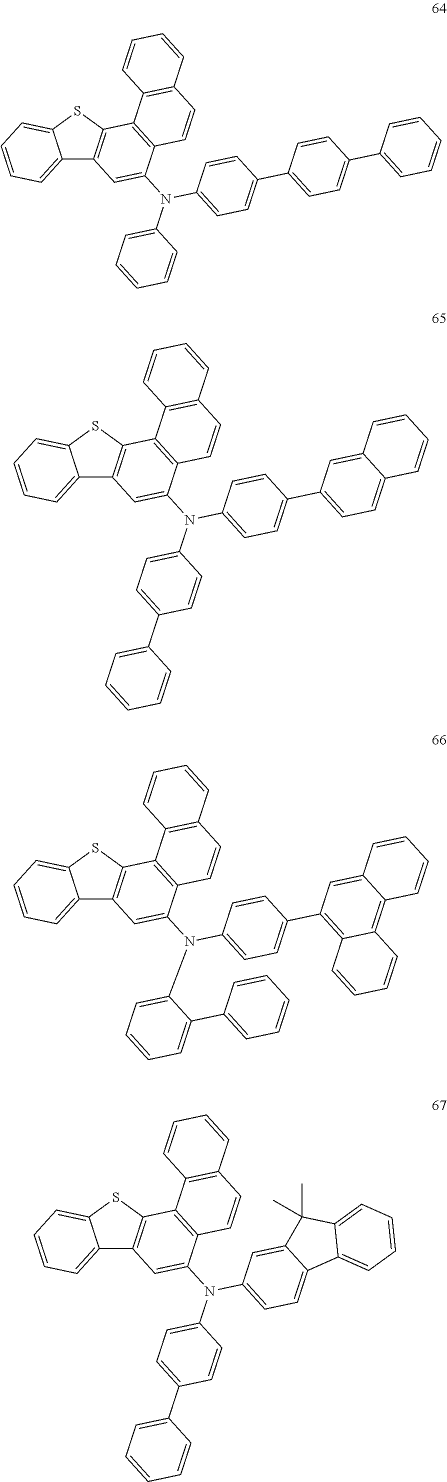 [SMILES: c1ccc(-c2ccc(-c3ccc(N(c4ccccc4)c4cc5c6ccccc6sc5c5c4ccc4ccccc45)cc3)cc2)cc1 ; c1ccc(-c2ccc(N(c3ccc(-c4ccc5ccccc5c4)cc3)c3cc4c5ccccc5sc4c4c3ccc3ccccc34)cc2)cc1 ; c1ccc(-c2ccccc2N(c2ccc(-c3cc4ccccc4c4ccccc34)cc2)c2cc3c4ccccc4sc3c3c2ccc2ccccc23)cc1 ; CC1(C)c2ccccc2-c2ccc(N(c3ccc(-c4ccccc4)cc3)c3cc4c5ccccc5sc4c4c3ccc3ccccc34)cc21]

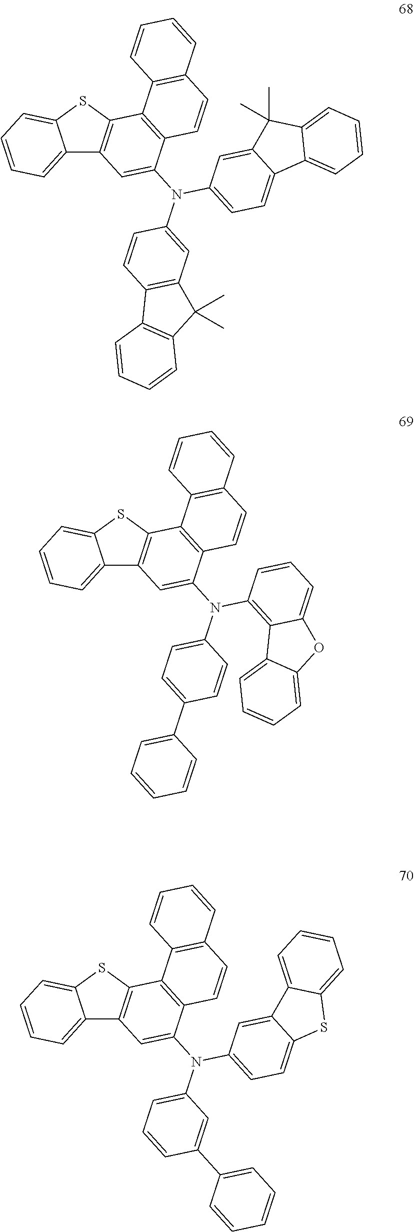 [SMILES: CC1(C)c2ccccc2-c2ccc(N(c3ccc4c(c3)C(C)(C)c3ccccc3-4)c3cc4c5ccccc5sc4c4c3ccc3ccccc34)cc21 ; c1ccc(-c2ccc(N(c3cc4c5ccccc5sc4c4c3ccc3ccccc34)c3cccc4oc5ccccc5c34)cc2)cc1 ; c1ccc(-c2cccc(N(c3ccc4sc5ccccc5c4c3)c3cc4c5ccccc5sc4c4c3ccc3ccccc34)c2)cc1]

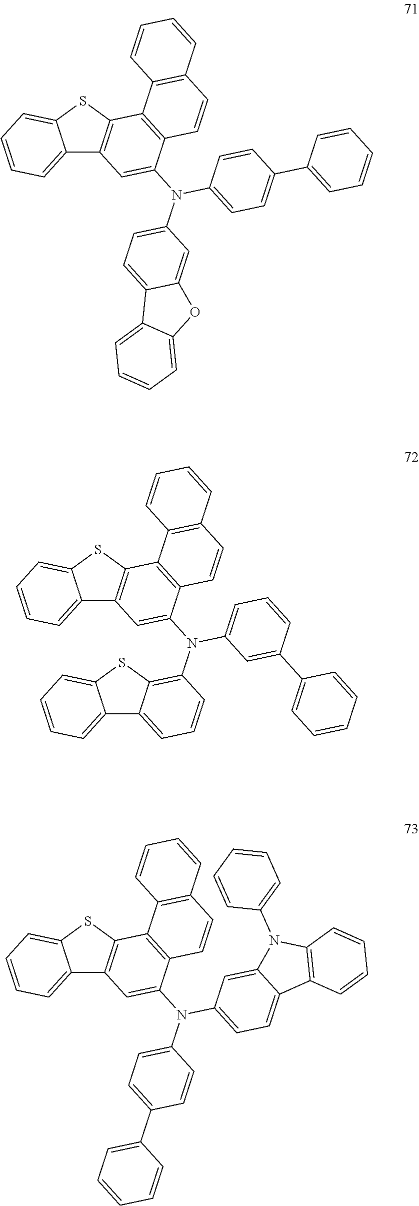 [SMILES: c1ccc(-c2ccc(N(c3ccc4c(c3)oc3ccccc34)c3cc4c5ccccc5sc4c4c3ccc3ccccc34)cc2)cc1 ; c1ccc(-c2cccc(N(c3cc4c5ccccc5sc4c4c3ccc3ccccc34)c3cccc4c3sc3ccccc34)c2)cc1 ; c1ccc(-c2ccc(N(c3ccc4c5ccccc5n(-c5ccccc5)c4c3)c3cc4c5ccccc5sc4c4c3ccc3ccccc34)cc2)cc1]

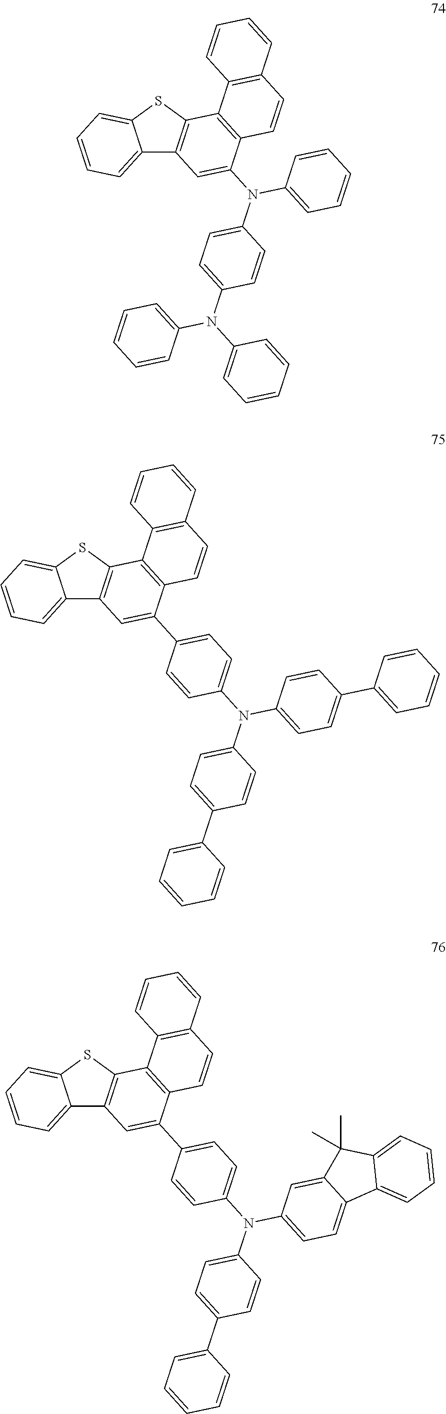 [SMILES: c1ccc(N(c2ccccc2)c2ccc(N(c3ccccc3)c3cc4c5ccccc5sc4c4c3ccc3ccccc34)cc2)cc1 ; c1ccc(-c2ccc(N(c3ccc(-c4ccccc4)cc3)c3ccc(-c4cc5c6ccccc6sc5c5c4ccc4ccccc45)cc3)cc2)cc1 ; CC1(C)c2ccccc2-c2ccc(N(c3ccc(-c4ccccc4)cc3)c3ccc(-c4cc5c6ccccc6sc5c5c4ccc4ccccc45)cc3)cc21]

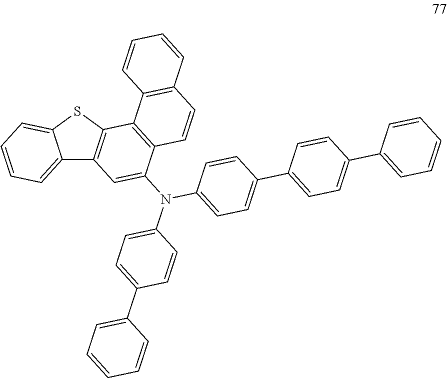 [SMILES: c1ccc(-c2ccc(-c3ccc(N(c4ccc(-c5ccccc5)cc4)c4cc5c6ccccc6sc5c5c4ccc4ccccc45)cc3)cc2)cc1]

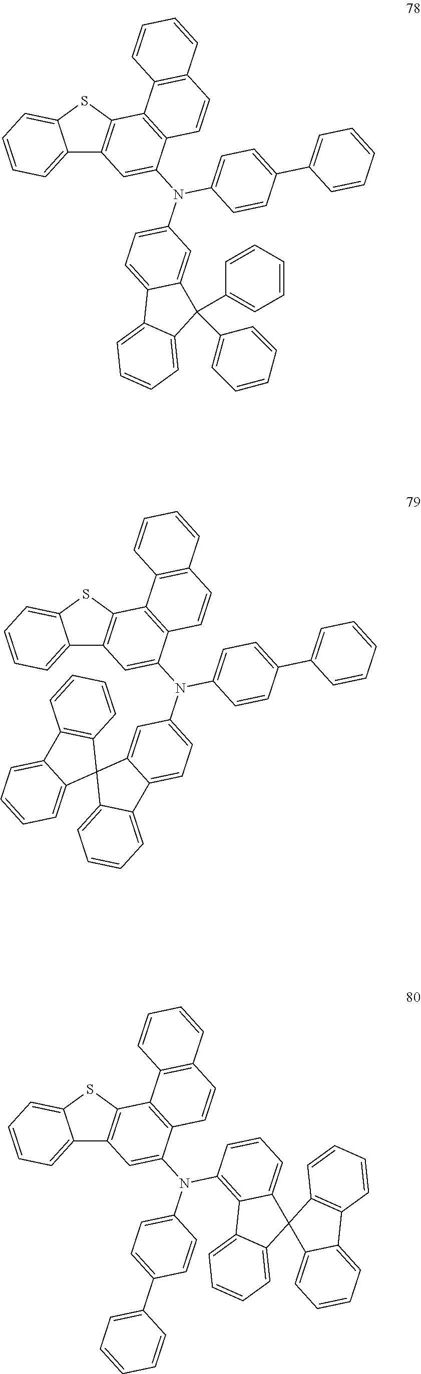 [SMILES: c1ccc(-c2ccc(N(c3ccc4c(c3)C(c3ccccc3)(c3ccccc3)c3ccccc3-4)c3cc4c5ccccc5sc4c4c3ccc3ccccc34)cc2)cc1 ; c1ccc(-c2ccc(N(c3ccc4c(c3)C3(c5ccccc5-c5ccccc53)c3ccccc3-4)c3cc4c5ccccc5sc4c4c3ccc3ccccc34)cc2)cc1 ; c1ccc(-c2ccc(N(c3cccc4c3-c3ccccc3C43c4ccccc4-c4ccccc43)c3cc4c5ccccc5sc4c4c3ccc3ccccc34)cc2)cc1]

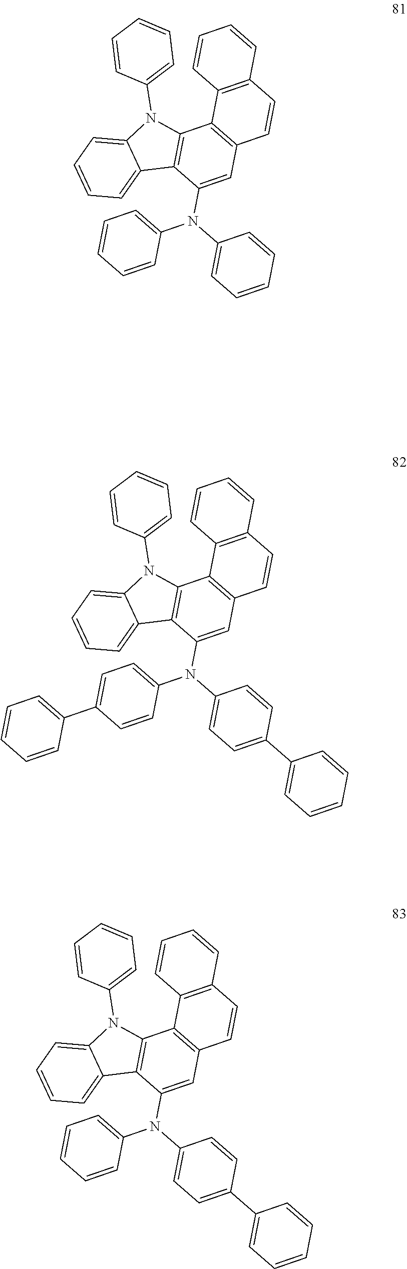 [SMILES: c1ccc(N(c2ccccc2)c2cc3ccc4ccccc4c3c3c2c2ccccc2n3-c2ccccc2)cc1 ; c1ccc(-c2ccc(N(c3ccc(-c4ccccc4)cc3)c3cc4ccc5ccccc5c4c4c3c3ccccc3n4-c3ccccc3)cc2)cc1 ; c1ccc(-c2ccc(N(c3ccccc3)c3cc4ccc5ccccc5c4c4c3c3ccccc3n4-c3ccccc3)cc2)cc1]

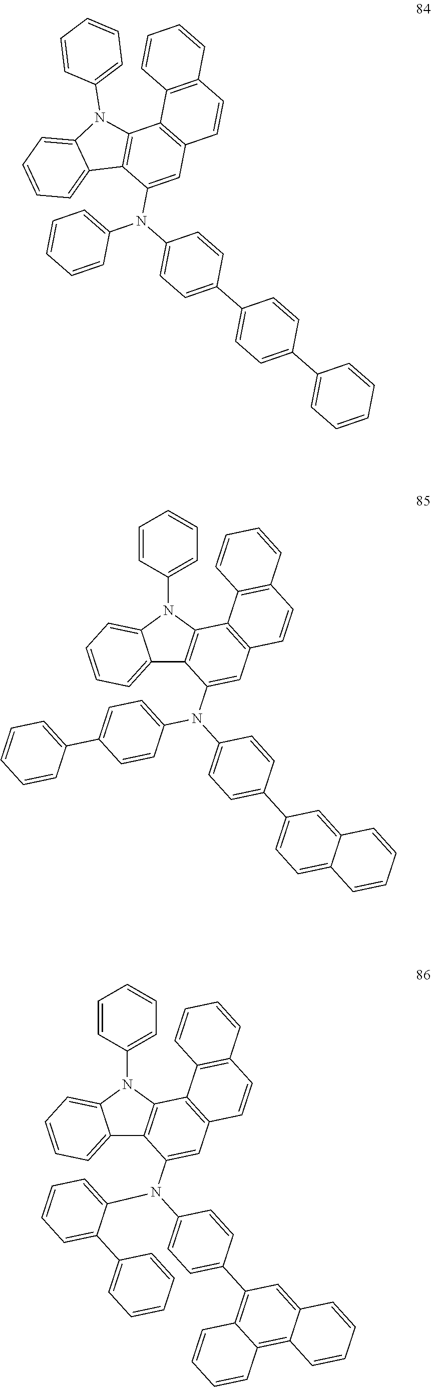 [SMILES: c1ccc(-c2ccc(-c3ccc(N(c4ccccc4)c4cc5ccc6ccccc6c5c5c4c4ccccc4n5-c4ccccc4)cc3)cc2)cc1 ; c1ccc(-c2ccc(N(c3ccc(-c4ccc5ccccc5c4)cc3)c3cc4ccc5ccccc5c4c4c3c3ccccc3n4-c3ccccc3)cc2)cc1 ; c1ccc(-c2ccccc2N(c2ccc(-c3cc4ccccc4c4ccccc34)cc2)c2cc3ccc4ccccc4c3c3c2c2ccccc2n3-c2ccccc2)cc1]

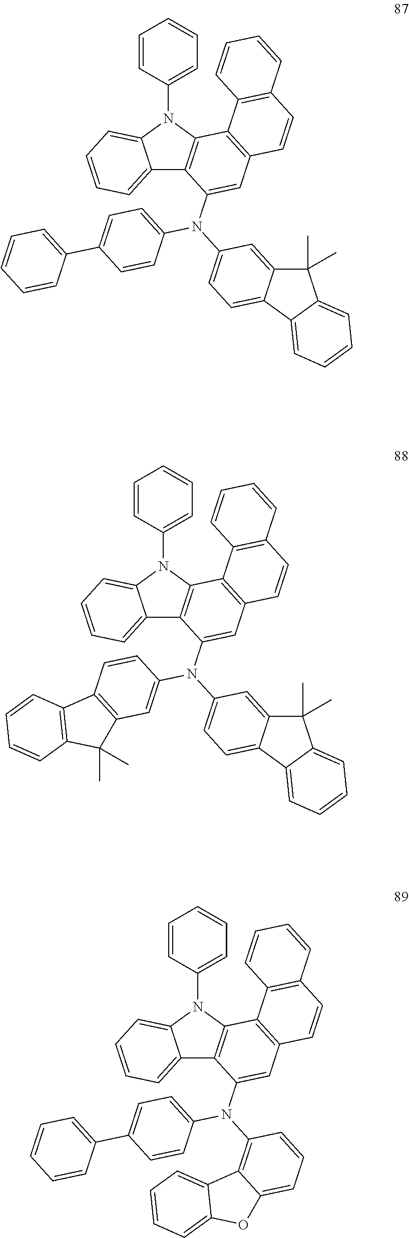 [SMILES: CC1(C)c2ccccc2-c2ccc(N(c3ccc(-c4ccccc4)cc3)c3cc4ccc5ccccc5c4c4c3c3ccccc3n4-c3ccccc3)cc21 ; CC1(C)c2ccccc2-c2ccc(N(c3ccc4c(c3)C(C)(C)c3ccccc3-4)c3cc4ccc5ccccc5c4c4c3c3ccccc3n4-c3ccccc3)cc21 ; c1ccc(-c2ccc(N(c3cccc4oc5ccccc5c34)c3cc4ccc5ccccc5c4c4c3c3ccccc3n4-c3ccccc3)cc2)cc1]

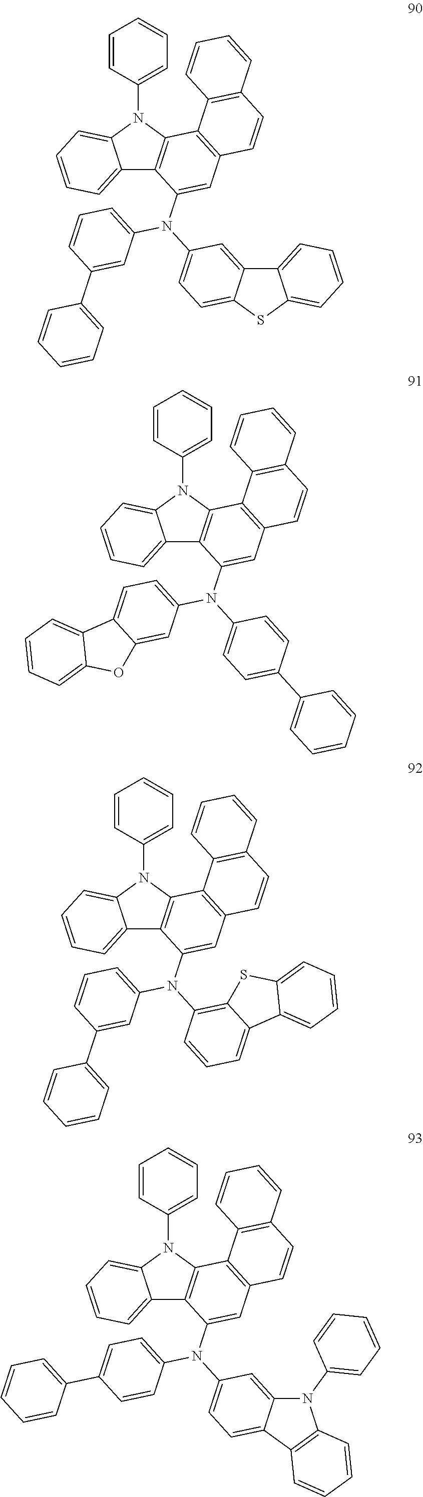 [SMILES: c1ccc(-c2cccc(N(c3ccc4sc5ccccc5c4c3)c3cc4ccc5ccccc5c4c4c3c3ccccc3n4-c3ccccc3)c2)cc1 ; c1ccc(-c2ccc(N(c3ccc4c(c3)oc3ccccc34)c3cc4ccc5ccccc5c4c4c3c3ccccc3n4-c3ccccc3)cc2)cc1 ; c1ccc(-c2cccc(N(c3cccc4c3sc3ccccc34)c3cc4ccc5ccccc5c4c4c3c3ccccc3n4-c3ccccc3)c2)cc1 ; c1ccc(-c2ccc(N(c3ccc4c5ccccc5n(-c5ccccc5)c4c3)c3cc4ccc5ccccc5c4c4c3c3ccccc3n4-c3ccccc3)cc2)cc1]

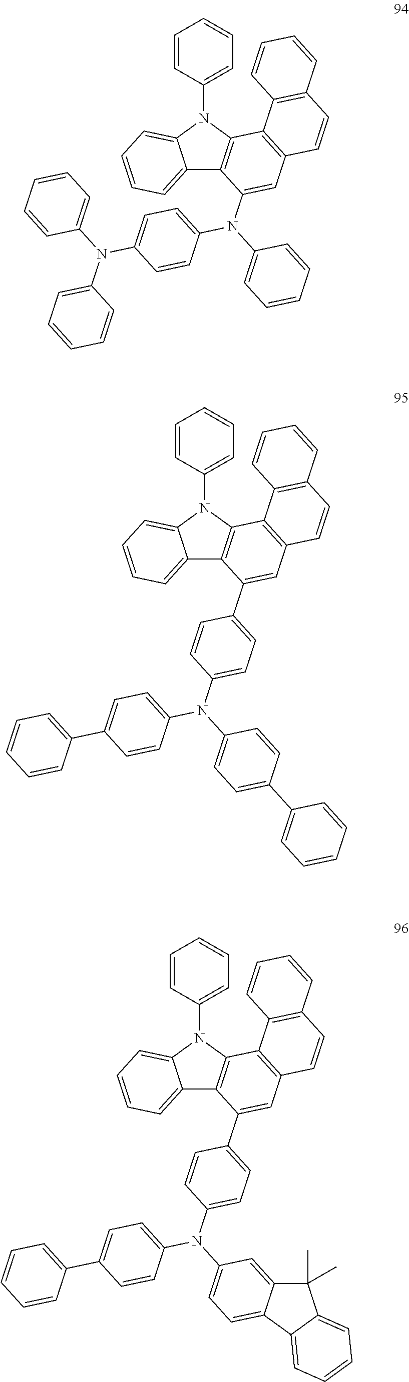 [SMILES: c1ccc(N(c2ccccc2)c2ccc(N(c3ccccc3)c3cc4ccc5ccccc5c4c4c3c3ccccc3n4-c3ccccc3)cc2)cc1 ; c1ccc(-c2ccc(N(c3ccc(-c4ccccc4)cc3)c3ccc(-c4cc5ccc6ccccc6c5c5c4c4ccccc4n5-c4ccccc4)cc3)cc2)cc1 ; CC1(C)c2ccccc2-c2ccc(N(c3ccc(-c4ccccc4)cc3)c3ccc(-c4cc5ccc6ccccc6c5c5c4c4ccccc4n5-c4ccccc4)cc3)cc21]

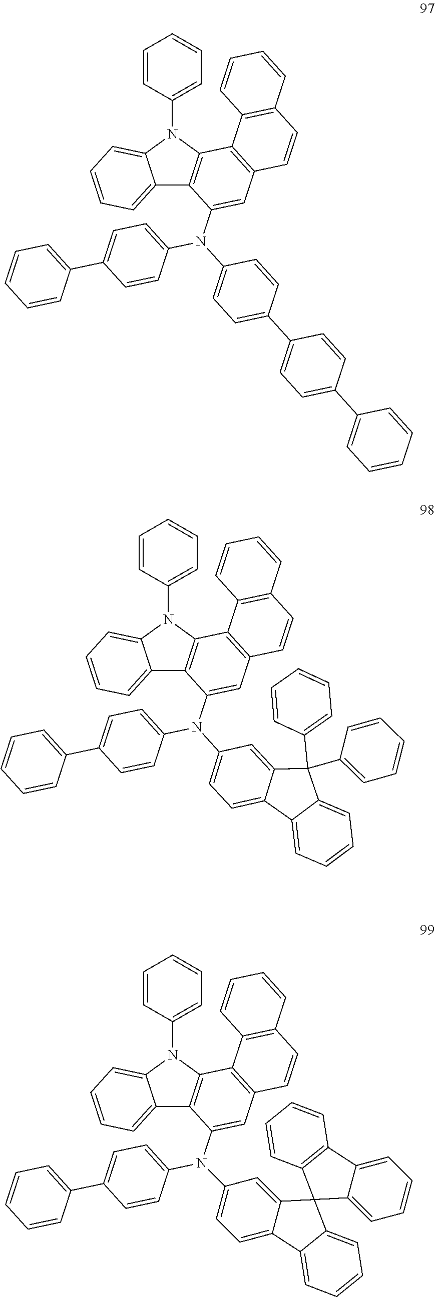 [SMILES: c1ccc(-c2ccc(-c3ccc(N(c4ccc(-c5ccccc5)cc4)c4cc5ccc6ccccc6c5c5c4c4ccccc4n5-c4ccccc4)cc3)cc2)cc1 ; c1ccc(-c2ccc(N(c3ccc4c(c3)C(c3ccccc3)(c3ccccc3)c3ccccc3-4)c3cc4ccc5ccccc5c4c4c3c3ccccc3n4-c3ccccc3)cc2)cc1 ; c1ccc(-c2ccc(N(c3ccc4c(c3)C3(c5ccccc5-c5ccccc53)c3ccccc3-4)c3cc4ccc5ccccc5c4c4c3c3ccccc3n4-c3ccccc3)cc2)cc1]

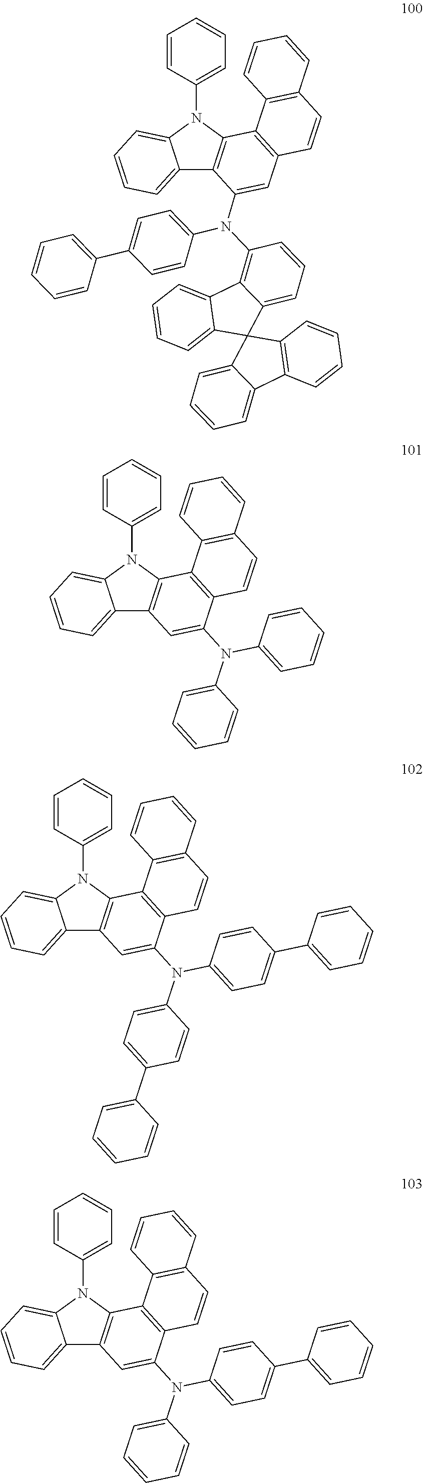

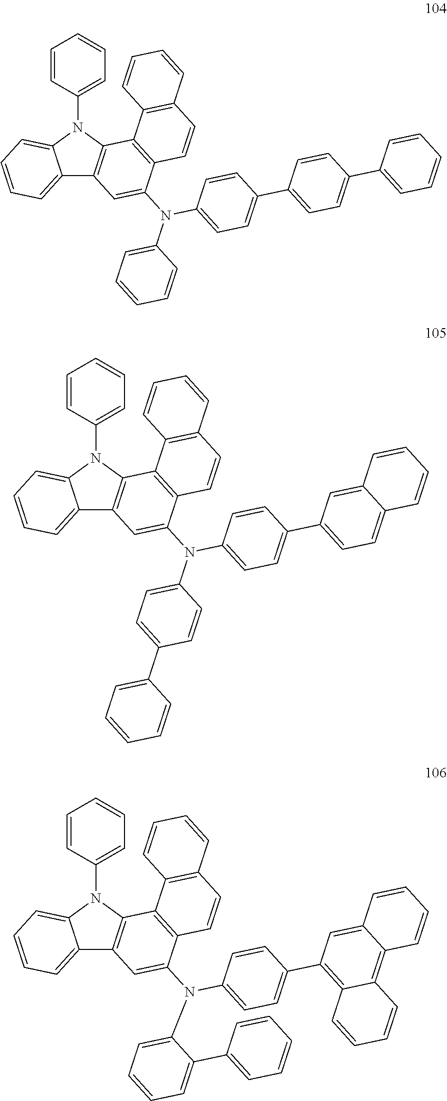 [SMILES: c1ccc(-c2ccc(-c3ccc(N(c4ccccc4)c4cc5c6ccccc6n(-c6ccccc6)c5c5c4ccc4ccccc45)cc3)cc2)cc1 ; c1ccc(-c2ccc(N(c3ccc(-c4ccc5ccccc5c4)cc3)c3cc4c5ccccc5n(-c5ccccc5)c4c4c3ccc3ccccc34)cc2)cc1 ; c1ccc(-c2ccccc2N(c2ccc(-c3cc4ccccc4c4ccccc34)cc2)c2cc3c4ccccc4n(-c4ccccc4)c3c3c2ccc2ccccc23)cc1]

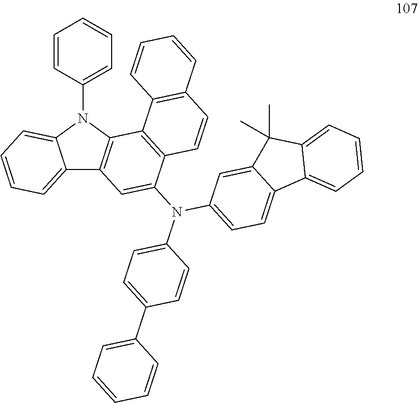 [SMILES: CC1(C)c2ccccc2-c2ccc(N(c3ccc(-c4ccccc4)cc3)c3cc4c5ccccc5n(-c5ccccc5)c4c4c3ccc3ccccc34)cc21]

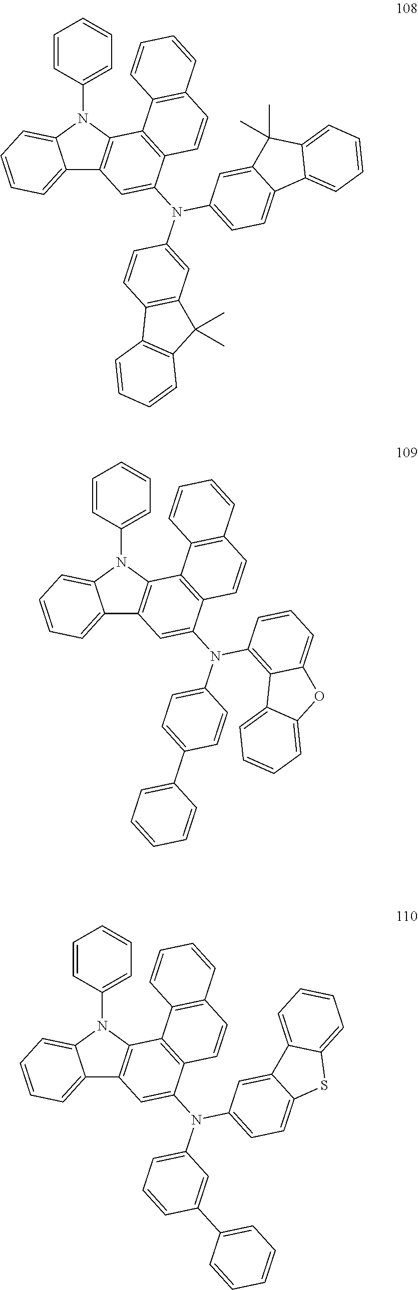 [SMILES: CC1(C)c2ccccc2-c2ccc(N(c3ccc4c(c3)C(C)(C)c3ccccc3-4)c3cc4c5ccccc5n(-c5ccccc5)c4c4c3ccc3ccccc34)cc21 ; c1ccc(-c2ccc(N(c3cc4c5ccccc5n(-c5ccccc5)c4c4c3ccc3ccccc34)c3cccc4oc5ccccc5c34)cc2)cc1 ; c1ccc(-c2cccc(N(c3ccc4sc5ccccc5c4c3)c3cc4c5ccccc5n(-c5ccccc5)c4c4c3ccc3ccccc34)c2)cc1]

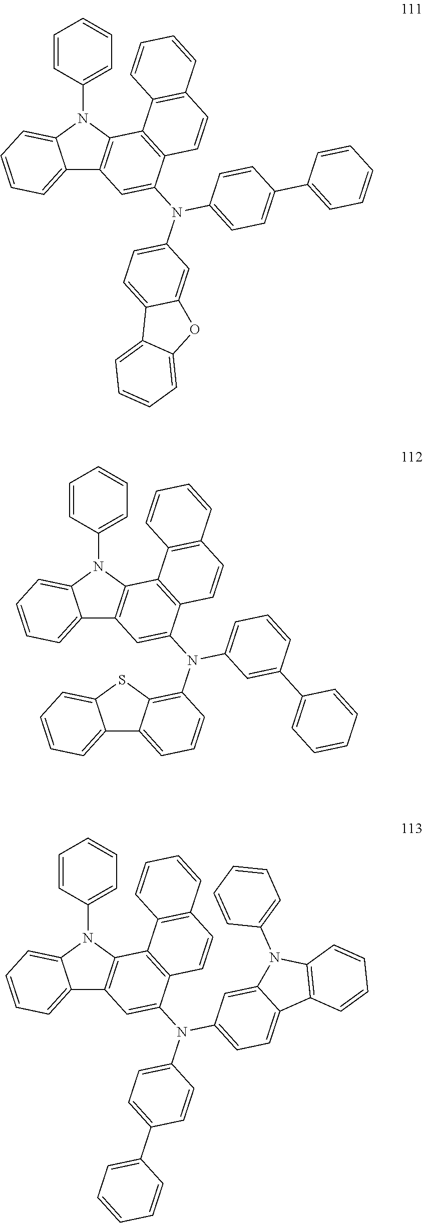 [SMILES: c1ccc(-c2ccc(N(c3ccc4c(c3)oc3ccccc34)c3cc4c5ccccc5n(-c5ccccc5)c4c4c3ccc3ccccc34)cc2)cc1 ; c1ccc(-c2cccc(N(c3cc4c5ccccc5n(-c5ccccc5)c4c4c3ccc3ccccc34)c3cccc4c3sc3ccccc34)c2)cc1 ; c1ccc(-c2ccc(N(c3ccc4c5ccccc5n(-c5ccccc5)c4c3)c3cc4c5ccccc5n(-c5ccccc5)c4c4c3ccc3ccccc34)cc2)cc1]

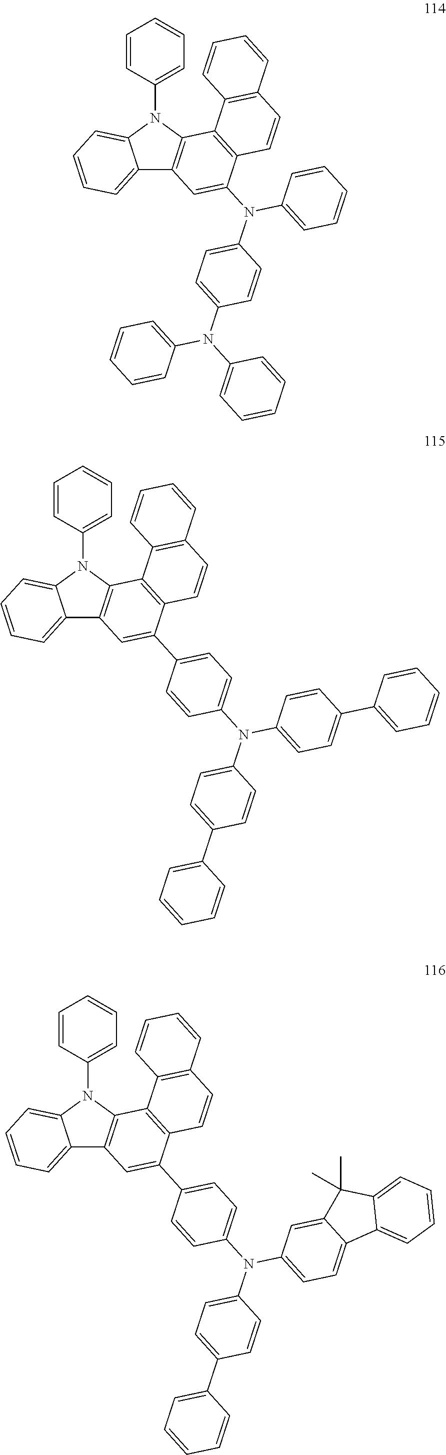 [SMILES: c1ccc(N(c2ccccc2)c2ccc(N(c3ccccc3)c3cc4c5ccccc5n(-c5ccccc5)c4c4c3ccc3ccccc34)cc2)cc1 ; c1ccc(-c2ccc(N(c3ccc(-c4ccccc4)cc3)c3ccc(-c4cc5c6ccccc6n(-c6ccccc6)c5c5c4ccc4ccccc45)cc3)cc2)cc1 ; CC1(C)c2ccccc2-c2ccc(N(c3ccc(-c4ccccc4)cc3)c3ccc(-c4cc5c6ccccc6n(-c6ccccc6)c5c5c4ccc4ccccc45)cc3)cc21]

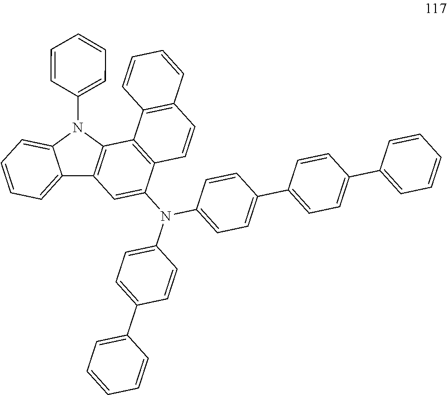 [SMILES: c1ccc(-c2ccc(-c3ccc(N(c4ccc(-c5ccccc5)cc4)c4cc5c6ccccc6n(-c6ccccc6)c5c5c4ccc4ccccc45)cc3)cc2)cc1]

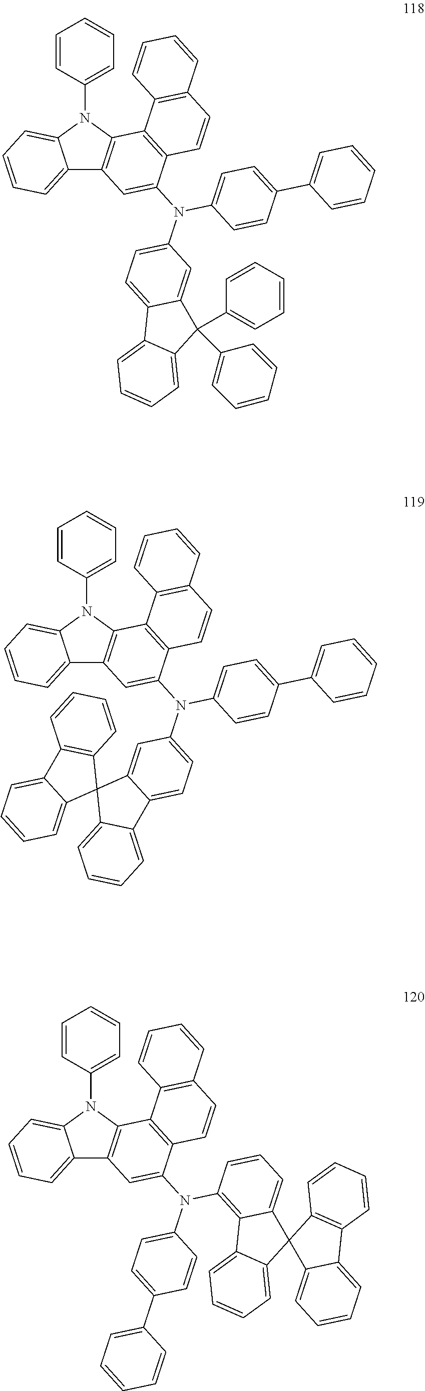 [SMILES: c1ccc(-c2ccc(N(c3ccc4c(c3)C(c3ccccc3)(c3ccccc3)c3ccccc3-4)c3cc4c5ccccc5n(-c5ccccc5)c4c4c3ccc3ccccc34)cc2)cc1 ; c1ccc(-c2ccc(N(c3ccc4c(c3)C3(c5ccccc5-c5ccccc53)c3ccccc3-4)c3cc4c5ccccc5n(-c5ccccc5)c4c4c3ccc3ccccc34)cc2)cc1 ; c1ccc(-c2ccc(N(c3cccc4c3-c3ccccc3C43c4ccccc4-c4ccccc43)c3cc4c5ccccc5n(-c5ccccc5)c4c4c3ccc3ccccc34)cc2)cc1]

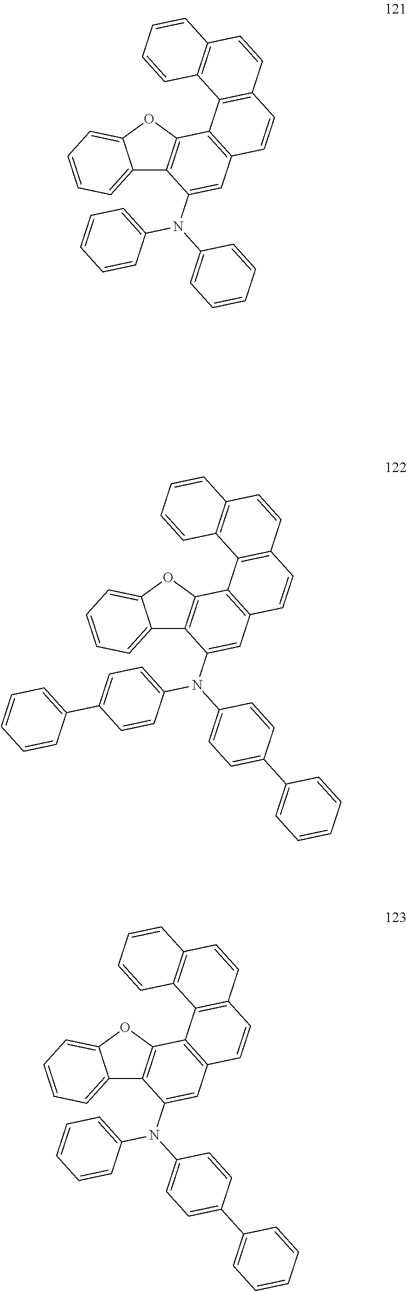 [SMILES: c1ccc(N(c2ccccc2)c2cc3ccc4ccc5ccccc5c4c3c3oc4ccccc4c23)cc1 ; c1ccc(-c2ccc(N(c3ccc(-c4ccccc4)cc3)c3cc4ccc5ccc6ccccc6c5c4c4oc5ccccc5c34)cc2)cc1 ; c1ccc(-c2ccc(N(c3ccccc3)c3cc4ccc5ccc6ccccc6c5c4c4oc5ccccc5c34)cc2)cc1]

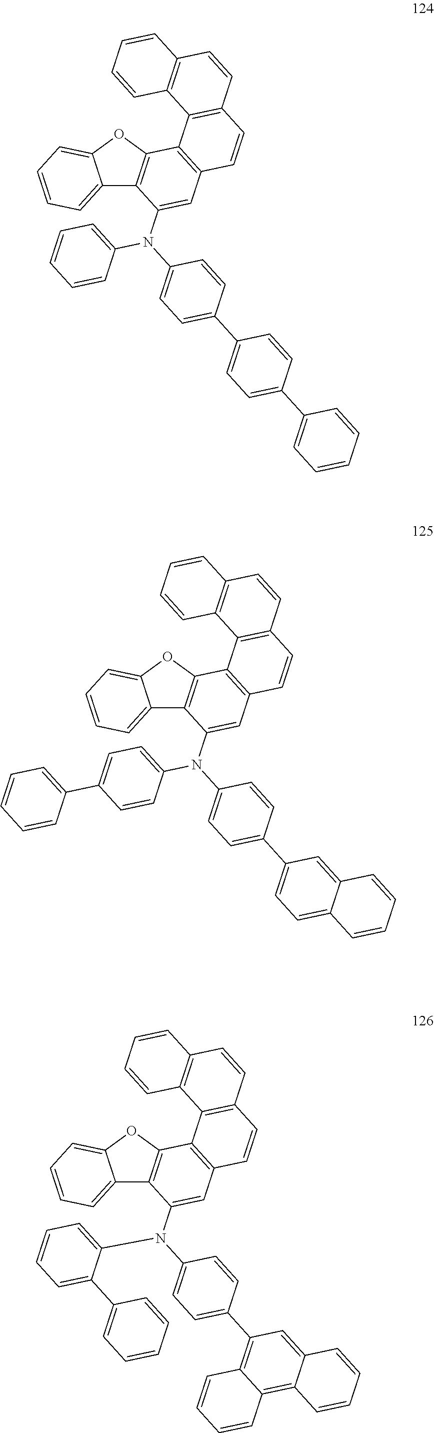 [SMILES: c1ccc(-c2ccc(-c3ccc(N(c4ccccc4)c4cc5ccc6ccc7ccccc7c6c5c5oc6ccccc6c45)cc3)cc2)cc1 ; c1ccc(-c2ccc(N(c3ccc(-c4ccc5ccccc5c4)cc3)c3cc4ccc5ccc6ccccc6c5c4c4oc5ccccc5c34)cc2)cc1 ; c1ccc(-c2ccccc2N(c2ccc(-c3cc4ccccc4c4ccccc34)cc2)c2cc3ccc4ccc5ccccc5c4c3c3oc4ccccc4c23)cc1]

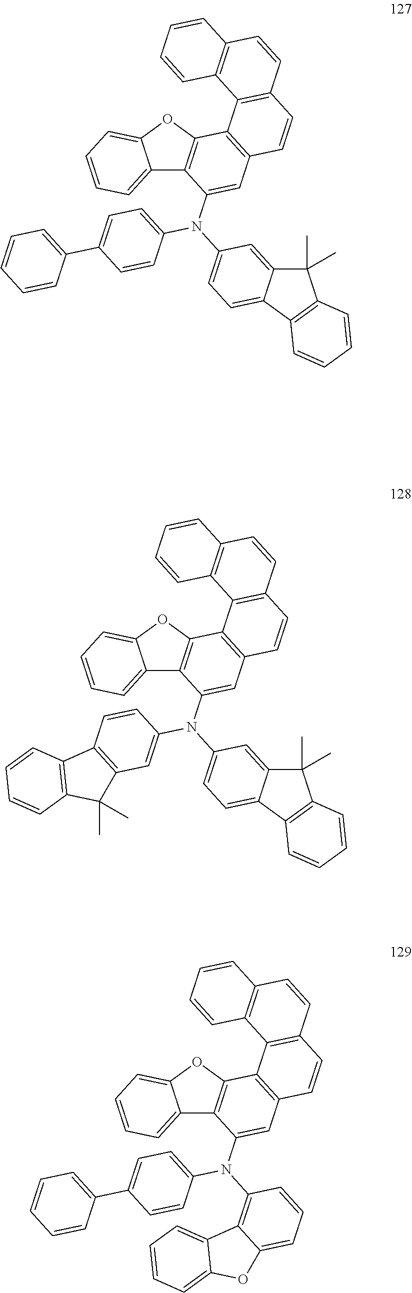 [SMILES: CC1(C)c2ccccc2-c2ccc(N(c3ccc(-c4ccccc4)cc3)c3cc4ccc5ccc6ccccc6c5c4c4oc5ccccc5c34)cc21 ; CC1(C)c2ccccc2-c2ccc(N(c3ccc4c(c3)C(C)(C)c3ccccc3-4)c3cc4ccc5ccc6ccccc6c5c4c4oc5ccccc5c34)cc21 ; c1ccc(-c2ccc(N(c3cccc4oc5ccccc5c34)c3cc4ccc5ccc6ccccc6c5c4c4oc5ccccc5c34)cc2)cc1]

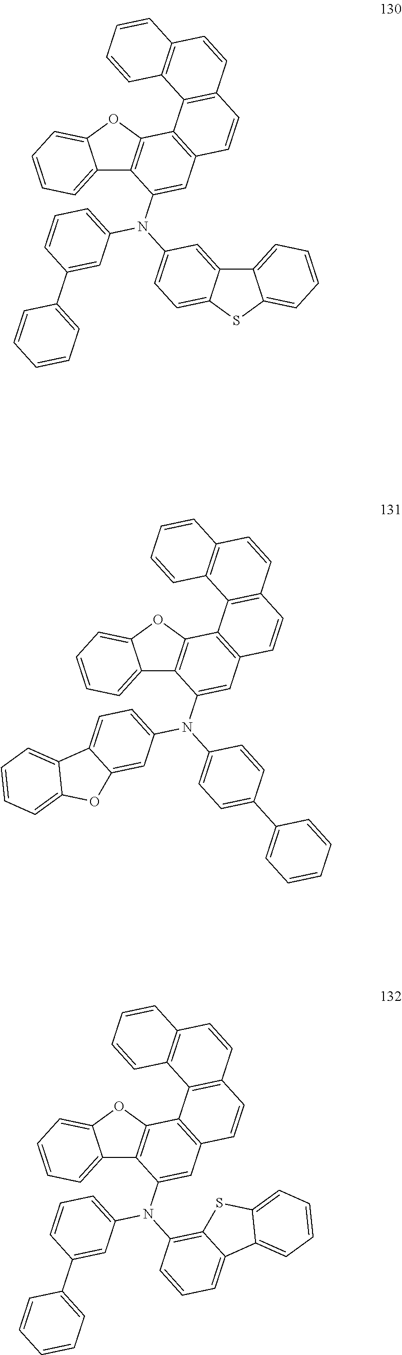 [SMILES: c1ccc(-c2cccc(N(c3ccc4sc5ccccc5c4c3)c3cc4ccc5ccc6ccccc6c5c4c4oc5ccccc5c34)c2)cc1 ; c1ccc(-c2ccc(N(c3ccc4c(c3)oc3ccccc34)c3cc4ccc5ccc6ccccc6c5c4c4oc5ccccc5c34)cc2)cc1 ; c1ccc(-c2cccc(N(c3cccc4c3sc3ccccc34)c3cc4ccc5ccc6ccccc6c5c4c4oc5ccccc5c34)c2)cc1]

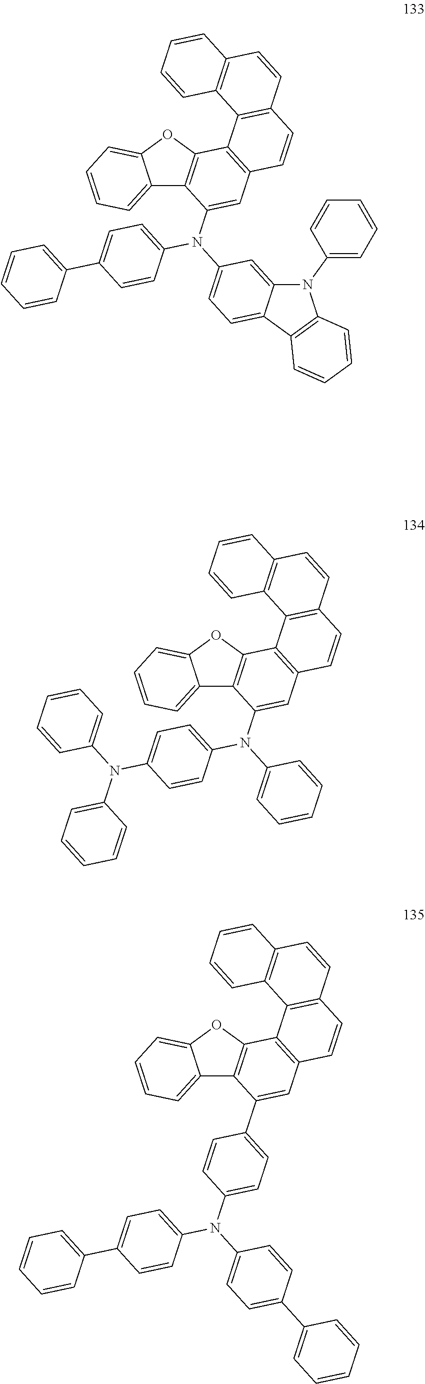 [SMILES: c1ccc(-c2ccc(N(c3ccc4c5ccccc5n(-c5ccccc5)c4c3)c3cc4ccc5ccc6ccccc6c5c4c4oc5ccccc5c34)cc2)cc1 ; c1ccc(N(c2ccccc2)c2ccc(N(c3ccccc3)c3cc4ccc5ccc6ccccc6c5c4c4oc5ccccc5c34)cc2)cc1 ; c1ccc(-c2ccc(N(c3ccc(-c4ccccc4)cc3)c3ccc(-c4cc5ccc6ccc7ccccc7c6c5c5oc6ccccc6c45)cc3)cc2)cc1]

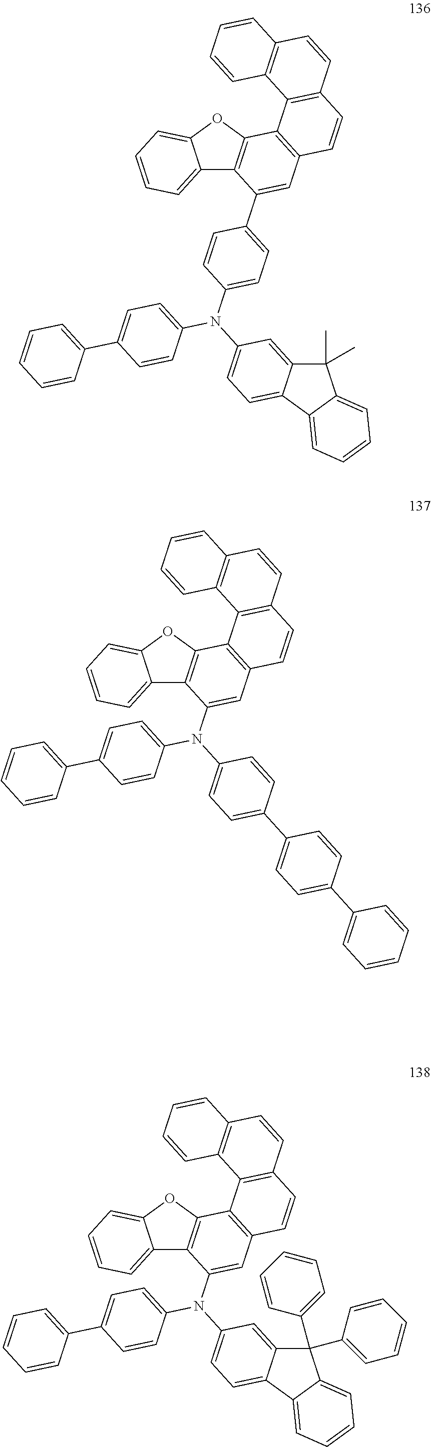 [SMILES: CC1(C)c2ccccc2-c2ccc(N(c3ccc(-c4ccccc4)cc3)c3ccc(-c4cc5ccc6ccc7ccccc7c6c5c5oc6ccccc6c45)cc3)cc21 ; c1ccc(-c2ccc(-c3ccc(N(c4ccc(-c5ccccc5)cc4)c4cc5ccc6ccc7ccccc7c6c5c5oc6ccccc6c45)cc3)cc2)cc1 ; c1ccc(-c2ccc(N(c3ccc4c(c3)C(c3ccccc3)(c3ccccc3)c3ccccc3-4)c3cc4ccc5ccc6ccccc6c5c4c4oc5ccccc5c34)cc2)cc1]

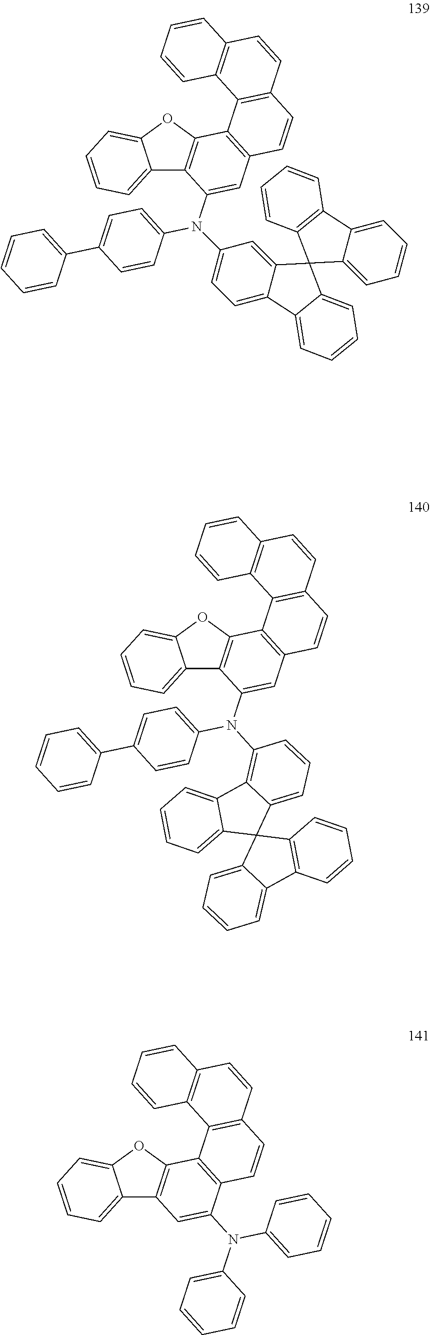 [SMILES: c1ccc(-c2ccc(N(c3ccc4c(c3)C3(c5ccccc5-c5ccccc53)c3ccccc3-4)c3cc4ccc5ccc6ccccc6c5c4c4oc5ccccc5c34)cc2)cc1 ; c1ccc(-c2ccc(N(c3cccc4c3-c3ccccc3C43c4ccccc4-c4ccccc43)c3cc4ccc5ccc6ccccc6c5c4c4oc5ccccc5c34)cc2)cc1 ; c1ccc(N(c2ccccc2)c2cc3c4ccccc4oc3c3c2ccc2ccc4ccccc4c23)cc1]

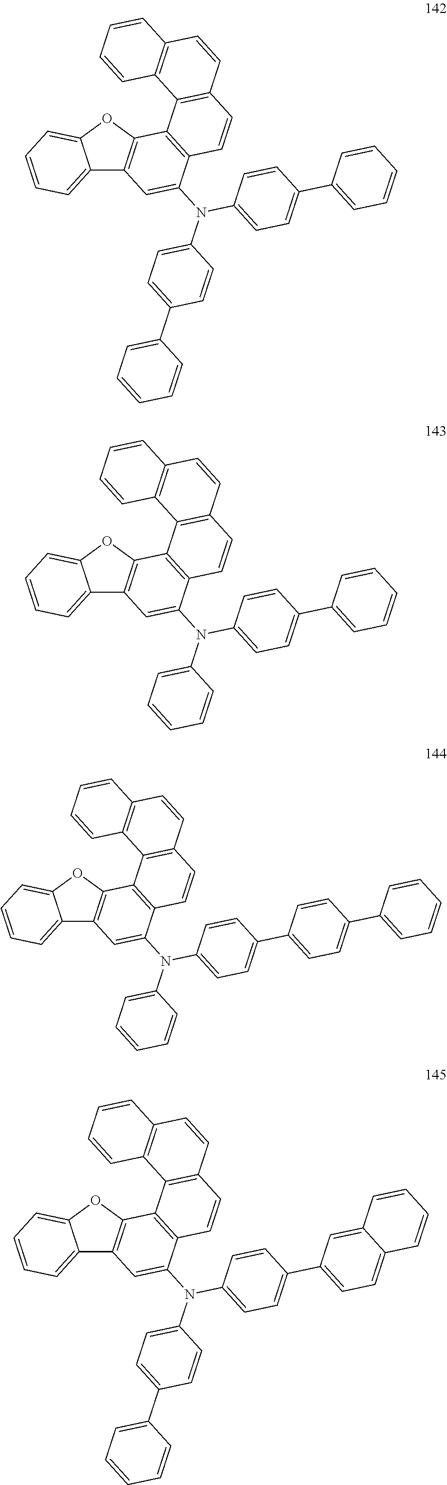

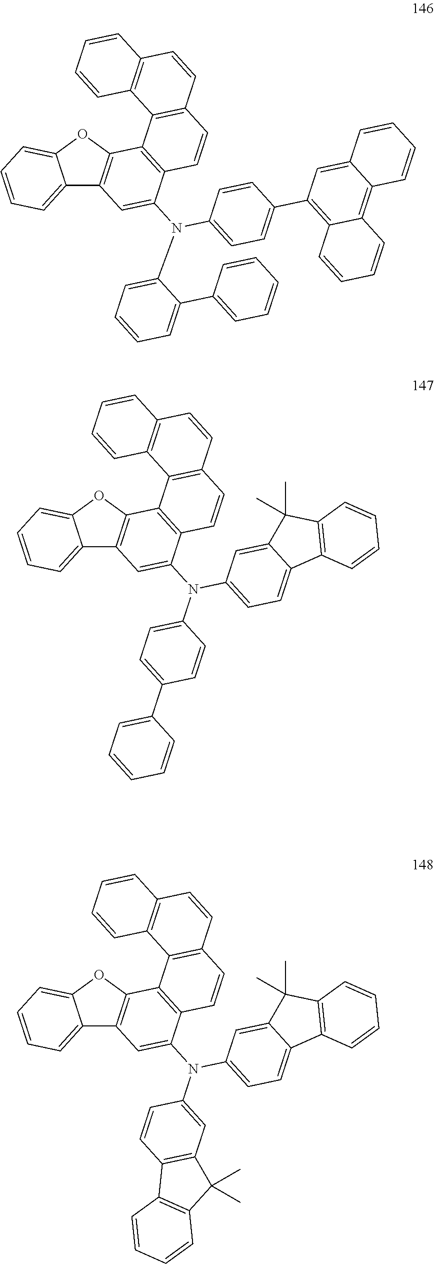 [SMILES: c1ccc(-c2ccccc2N(c2ccc(-c3cc4ccccc4c4ccccc34)cc2)c2cc3c4ccccc4oc3c3c2ccc2ccc4ccccc4c23)cc1 ; CC1(C)c2ccccc2-c2ccc(N(c3ccc(-c4ccccc4)cc3)c3cc4c5ccccc5oc4c4c3ccc3ccc5ccccc5c34)cc21 ; CC1(C)c2ccccc2-c2ccc(N(c3ccc4c(c3)C(C)(C)c3ccccc3-4)c3cc4c5ccccc5oc4c4c3ccc3ccc5ccccc5c34)cc21]

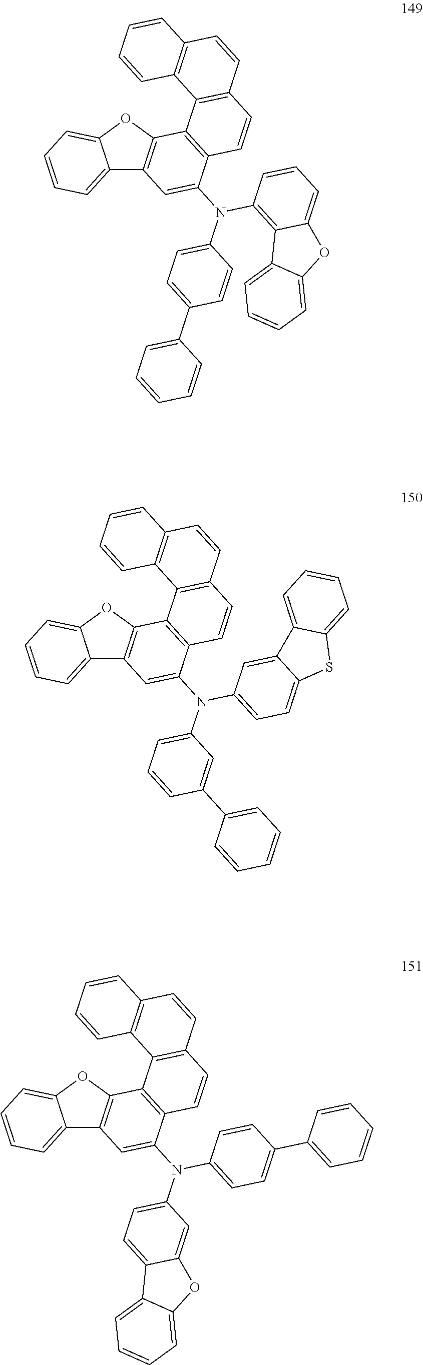 [SMILES: c1ccc(-c2ccc(N(c3cc4c5ccccc5oc4c4c3ccc3ccc5ccccc5c34)c3cccc4oc5ccccc5c34)cc2)cc1 ; c1ccc(-c2cccc(N(c3ccc4sc5ccccc5c4c3)c3cc4c5ccccc5oc4c4c3ccc3ccc5ccccc5c34)c2)cc1 ; c1ccc(-c2ccc(N(c3ccc4c(c3)oc3ccccc34)c3cc4c5ccccc5oc4c4c3ccc3ccc5ccccc5c34)cc2)cc1]

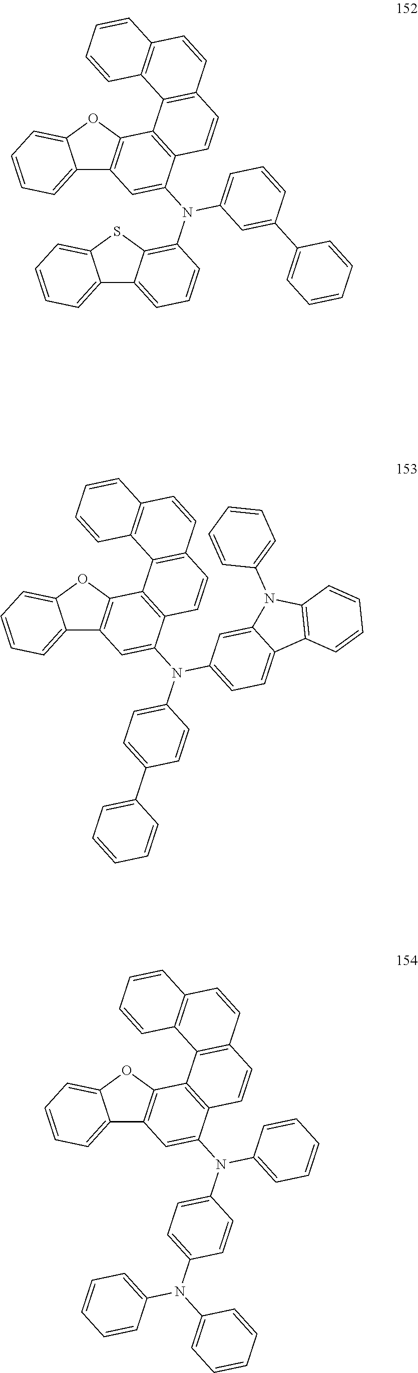 [SMILES: c1ccc(-c2cccc(N(c3cc4c5ccccc5oc4c4c3ccc3ccc5ccccc5c34)c3cccc4c3sc3ccccc34)c2)cc1 ; c1ccc(-c2ccc(N(c3ccc4c5ccccc5n(-c5ccccc5)c4c3)c3cc4c5ccccc5oc4c4c3ccc3ccc5ccccc5c34)cc2)cc1 ; c1ccc(N(c2ccccc2)c2ccc(N(c3ccccc3)c3cc4c5ccccc5oc4c4c3ccc3ccc5ccccc5c34)cc2)cc1]

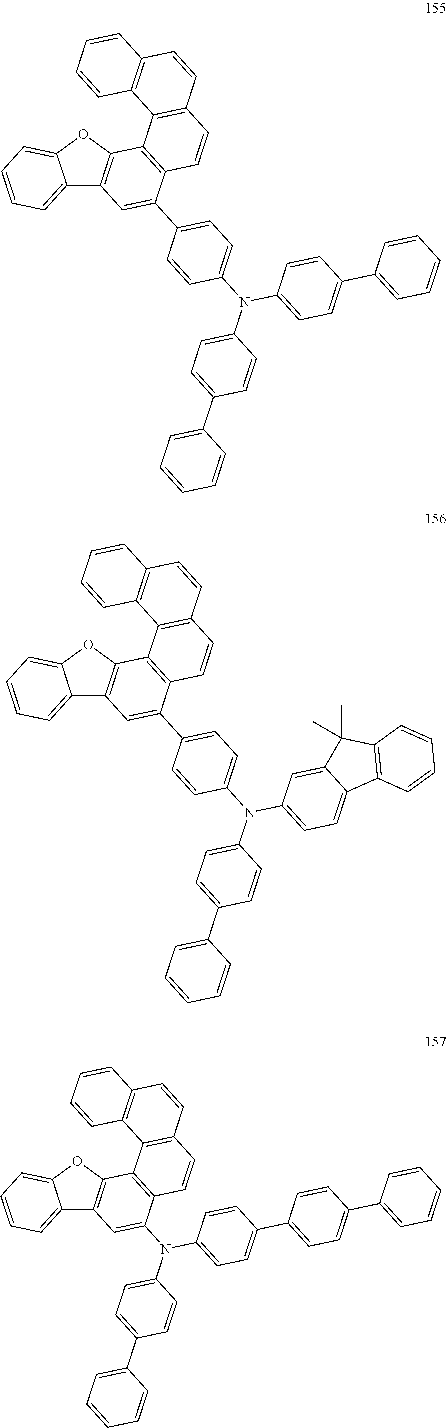 [SMILES: c1ccc(-c2ccc(N(c3ccc(-c4ccccc4)cc3)c3ccc(-c4cc5c6ccccc6oc5c5c4ccc4ccc6ccccc6c45)cc3)cc2)cc1 ; CC1(C)c2ccccc2-c2ccc(N(c3ccc(-c4ccccc4)cc3)c3ccc(-c4cc5c6ccccc6oc5c5c4ccc4ccc6ccccc6c45)cc3)cc21 ; c1ccc(-c2ccc(-c3ccc(N(c4ccc(-c5ccccc5)cc4)c4cc5c6ccccc6oc5c5c4ccc4ccc6ccccc6c45)cc3)cc2)cc1]

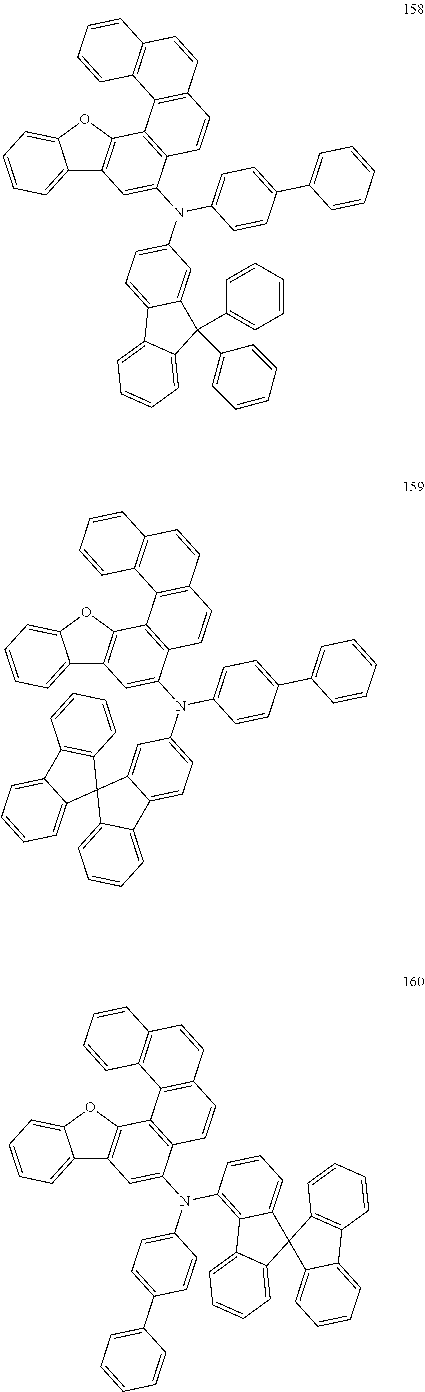 [SMILES: c1ccc(-c2ccc(N(c3ccc4c(c3)C(c3ccccc3)(c3ccccc3)c3ccccc3-4)c3cc4c5ccccc5oc4c4c3ccc3ccc5ccccc5c34)cc2)cc1 ; c1ccc(-c2ccc(N(c3ccc4c(c3)C3(c5ccccc5-c5ccccc53)c3ccccc3-4)c3cc4c5ccccc5oc4c4c3ccc3ccc5ccccc5c34)cc2)cc1 ; c1ccc(-c2ccc(N(c3cccc4c3-c3ccccc3C43c4ccccc4-c4ccccc43)c3cc4c5ccccc5oc4c4c3ccc3ccc5ccccc5c34)cc2)cc1]

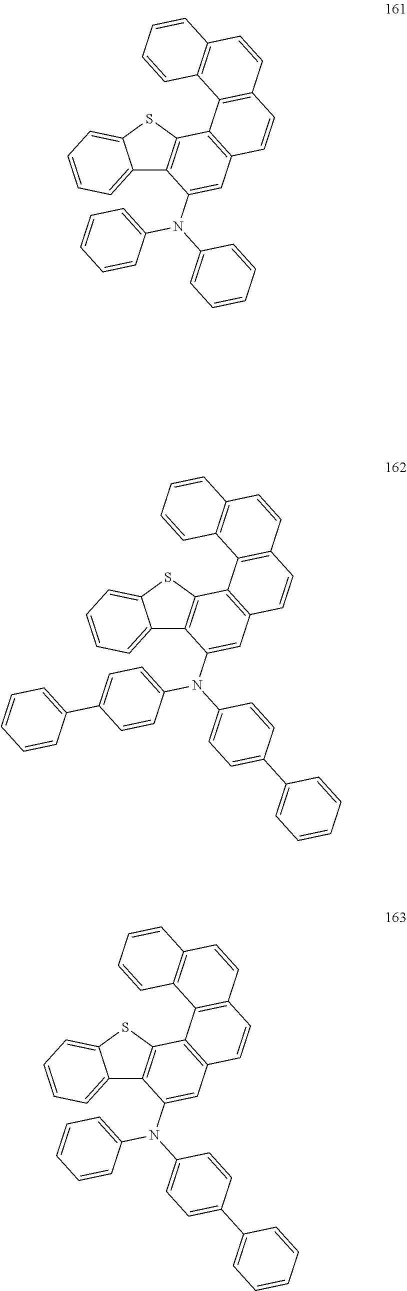 [SMILES: c1ccc(N(c2ccccc2)c2cc3ccc4ccc5ccccc5c4c3c3sc4ccccc4c23)cc1 ; c1ccc(-c2ccc(N(c3ccc(-c4ccccc4)cc3)c3cc4ccc5ccc6ccccc6c5c4c4sc5ccccc5c34)cc2)cc1 ; c1ccc(-c2ccc(N(c3ccccc3)c3cc4ccc5ccc6ccccc6c5c4c4sc5ccccc5c34)cc2)cc1]

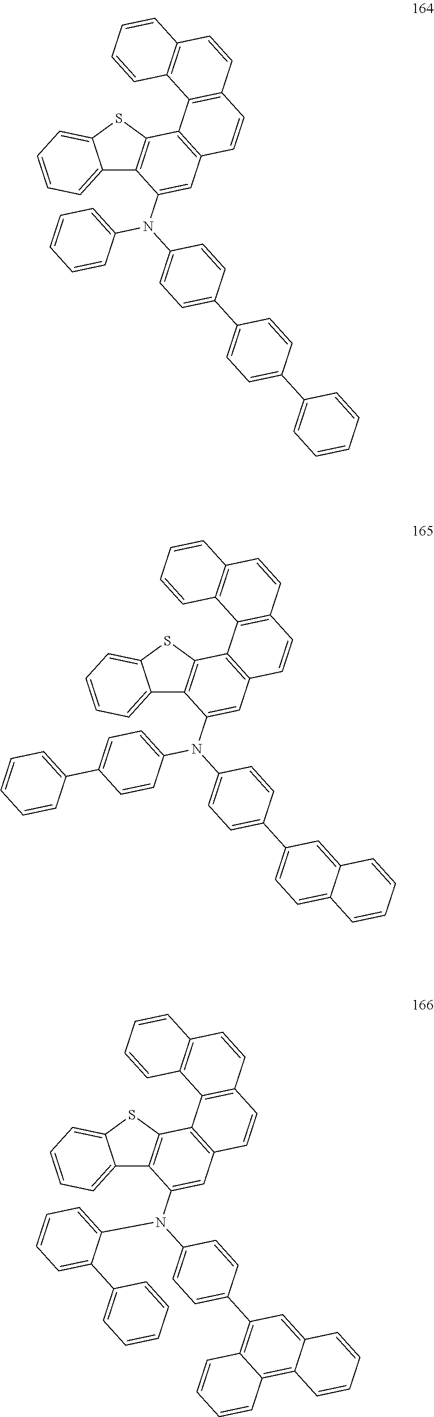 [SMILES: c1ccc(-c2ccc(-c3ccc(N(c4ccccc4)c4cc5ccc6ccc7ccccc7c6c5c5sc6ccccc6c45)cc3)cc2)cc1 ; c1ccc(-c2ccc(N(c3ccc(-c4ccc5ccccc5c4)cc3)c3cc4ccc5ccc6ccccc6c5c4c4sc5ccccc5c34)cc2)cc1 ; c1ccc(-c2ccccc2N(c2ccc(-c3cc4ccccc4c4ccccc34)cc2)c2cc3ccc4ccc5ccccc5c4c3c3sc4ccccc4c23)cc1]

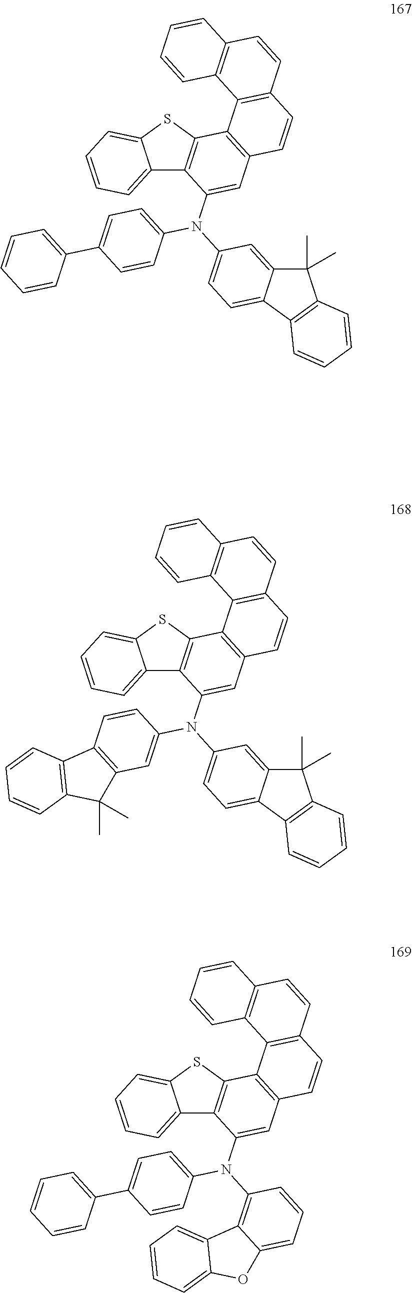 [SMILES: CC1(C)c2ccccc2-c2ccc(N(c3ccc(-c4ccccc4)cc3)c3cc4ccc5ccc6ccccc6c5c4c4sc5ccccc5c34)cc21 ; CC1(C)c2ccccc2-c2ccc(N(c3ccc4c(c3)C(C)(C)c3ccccc3-4)c3cc4ccc5ccc6ccccc6c5c4c4sc5ccccc5c34)cc21 ; c1ccc(-c2ccc(N(c3cccc4oc5ccccc5c34)c3cc4ccc5ccc6ccccc6c5c4c4sc5ccccc5c34)cc2)cc1]

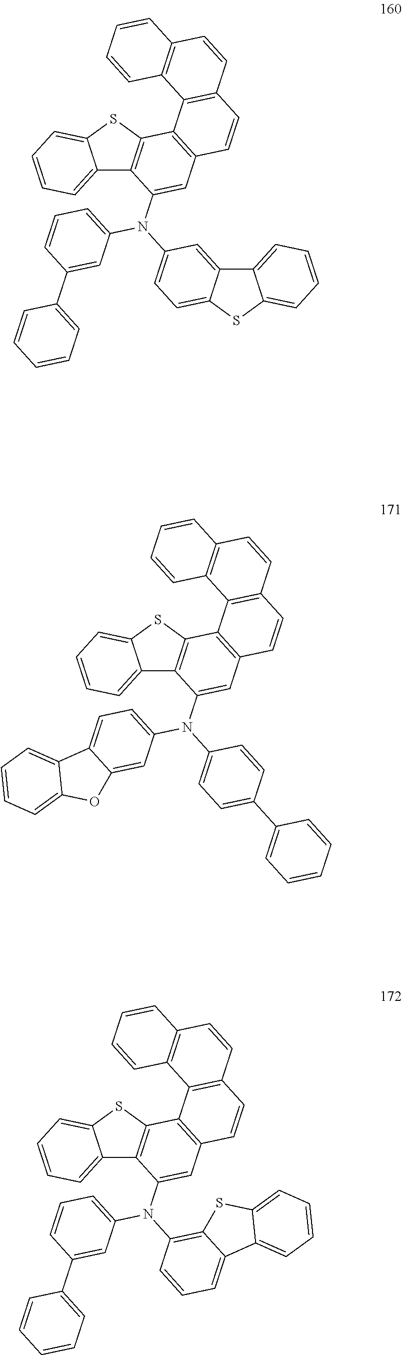 [SMILES: c1ccc(-c2cccc(N(c3ccc4sc5ccccc5c4c3)c3cc4ccc5ccc6ccccc6c5c4c4sc5ccccc5c34)c2)cc1 ; c1ccc(-c2ccc(N(c3ccc4c(c3)oc3ccccc34)c3cc4ccc5ccc6ccccc6c5c4c4sc5ccccc5c34)cc2)cc1 ; c1ccc(-c2cccc(N(c3cccc4c3sc3ccccc34)c3cc4ccc5ccc6ccccc6c5c4c4sc5ccccc5c34)c2)cc1]

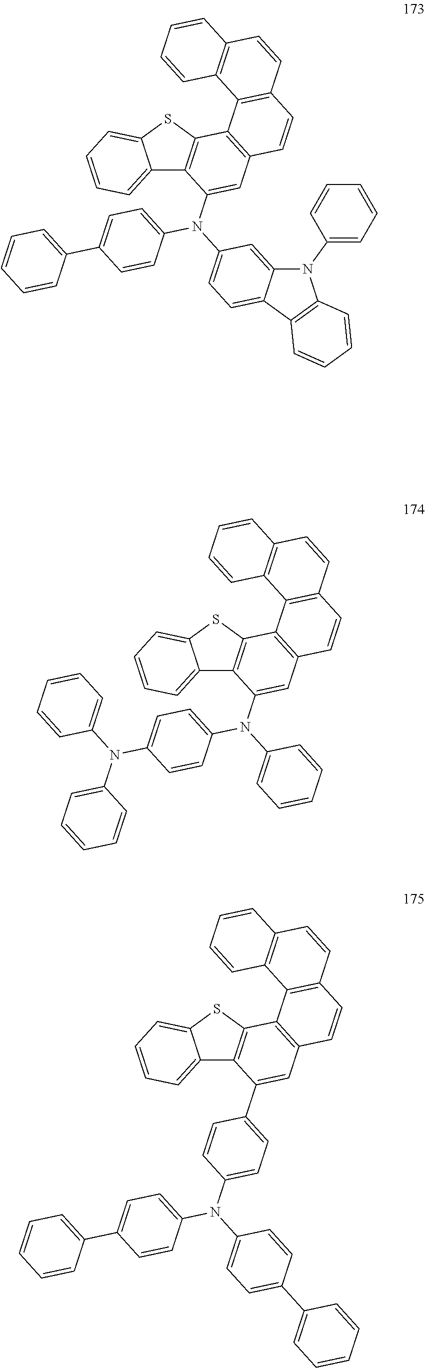 [SMILES: c1ccc(-c2ccc(N(c3ccc4c5ccccc5n(-c5ccccc5)c4c3)c3cc4ccc5ccc6ccccc6c5c4c4sc5ccccc5c34)cc2)cc1 ; c1ccc(N(c2ccccc2)c2ccc(N(c3ccccc3)c3cc4ccc5ccc6ccccc6c5c4c4sc5ccccc5c34)cc2)cc1 ; c1ccc(-c2ccc(N(c3ccc(-c4ccccc4)cc3)c3ccc(-c4cc5ccc6ccc7ccccc7c6c5c5sc6ccccc6c45)cc3)cc2)cc1]

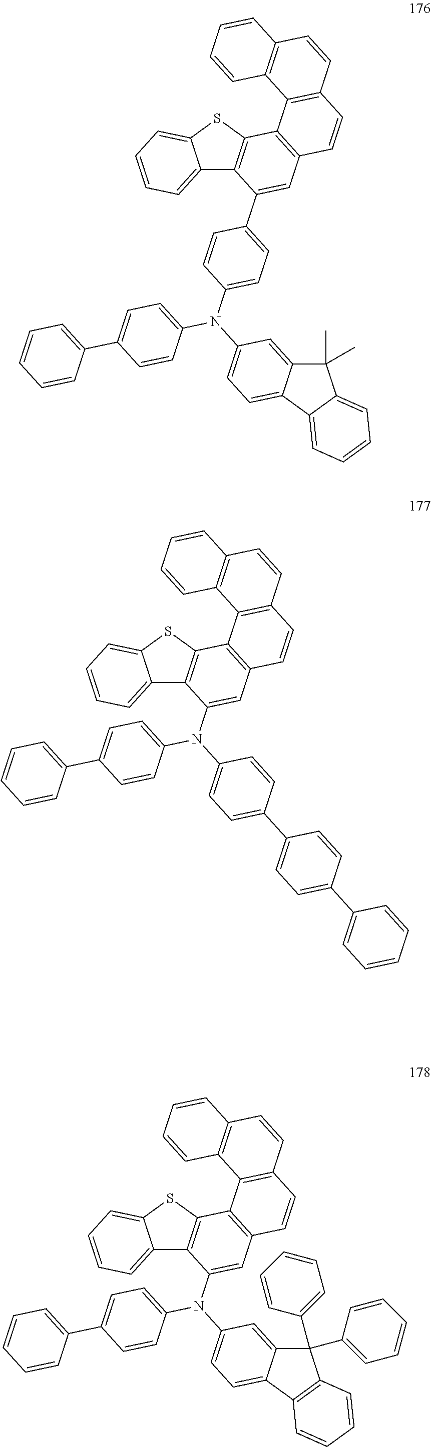 [SMILES: CC1(C)c2ccccc2-c2ccc(N(c3ccc(-c4ccccc4)cc3)c3ccc(-c4cc5ccc6ccc7ccccc7c6c5c5sc6ccccc6c45)cc3)cc21 ; c1ccc(-c2ccc(-c3ccc(N(c4ccc(-c5ccccc5)cc4)c4cc5ccc6ccc7ccccc7c6c5c5sc6ccccc6c45)cc3)cc2)cc1 ; c1ccc(-c2ccc(N(c3ccc4c(c3)C(c3ccccc3)(c3ccccc3)c3ccccc3-4)c3cc4ccc5ccc6ccccc6c5c4c4sc5ccccc5c34)cc2)cc1]

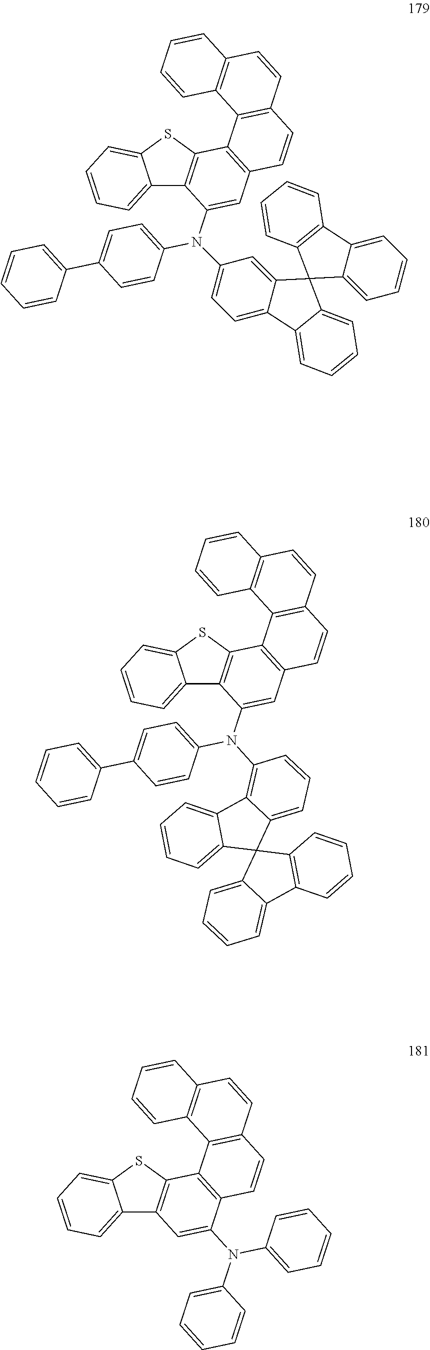 [SMILES: c1ccc(-c2ccc(N(c3ccc4c(c3)C3(c5ccccc5-c5ccccc53)c3ccccc3-4)c3cc4ccc5ccc6ccccc6c5c4c4sc5ccccc5c34)cc2)cc1 ; c1ccc(-c2ccc(N(c3cccc4c3-c3ccccc3C43c4ccccc4-c4ccccc43)c3cc4ccc5ccc6ccccc6c5c4c4sc5ccccc5c34)cc2)cc1 ; c1ccc(N(c2ccccc2)c2cc3c4ccccc4sc3c3c2ccc2ccc4ccccc4c23)cc1]

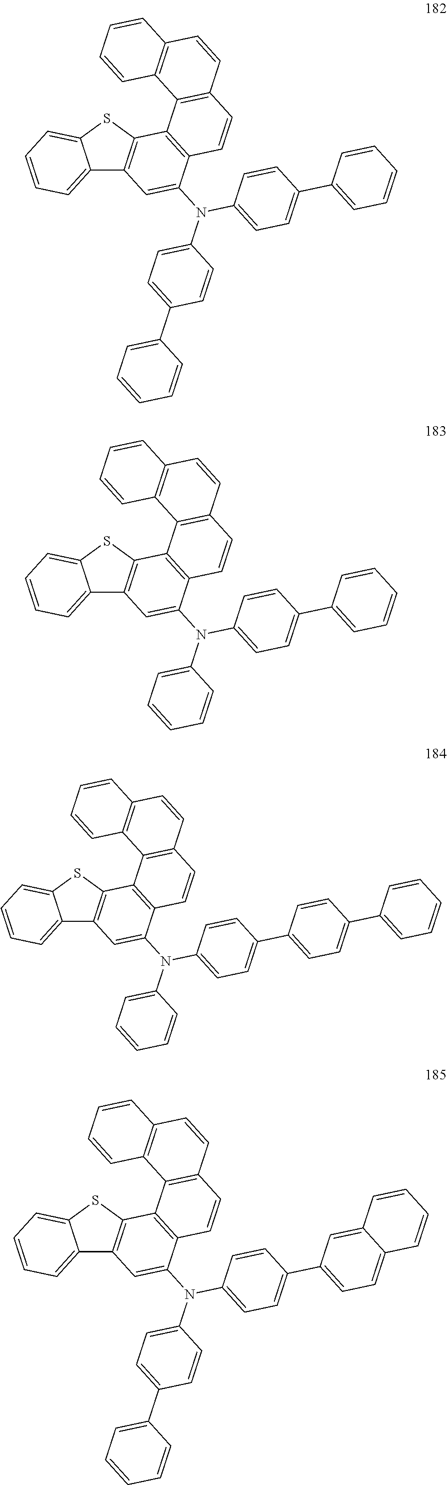

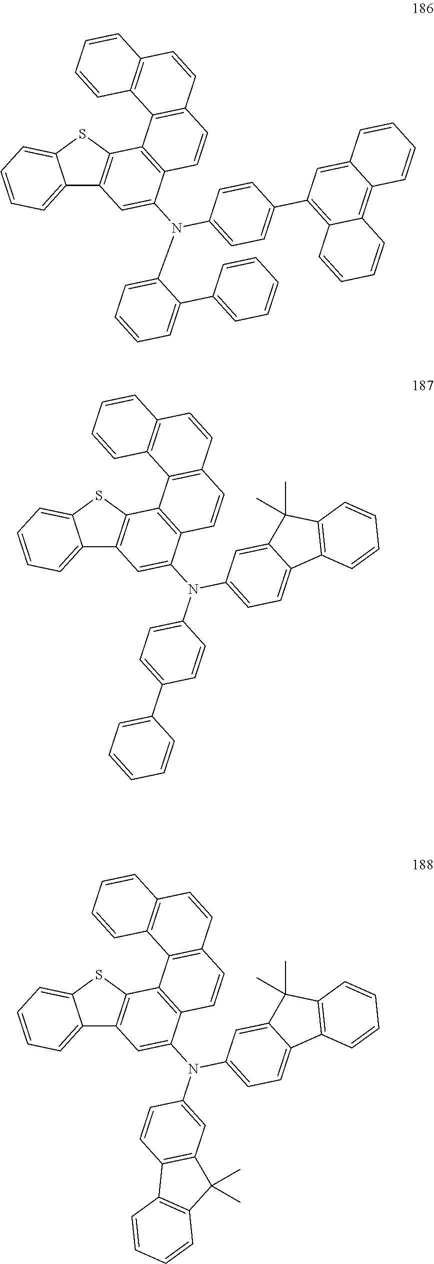 [SMILES: c1ccc(-c2ccccc2N(c2ccc(-c3cc4ccccc4c4ccccc34)cc2)c2cc3c4ccccc4sc3c3c2ccc2ccc4ccccc4c23)cc1 ; CC1(C)c2ccccc2-c2ccc(N(c3ccc(-c4ccccc4)cc3)c3cc4c5ccccc5sc4c4c3ccc3ccc5ccccc5c34)cc21 ; CC1(C)c2ccccc2-c2ccc(N(c3ccc4c(c3)C(C)(C)c3ccccc3-4)c3cc4c5ccccc5sc4c4c3ccc3ccc5ccccc5c34)cc21]

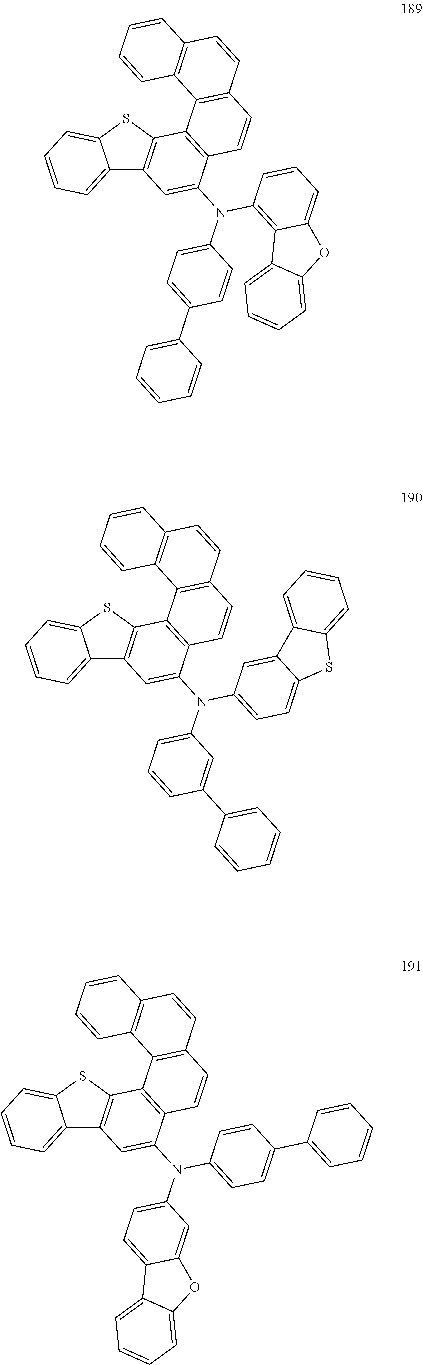 [SMILES: c1ccc(-c2ccc(N(c3cc4c5ccccc5sc4c4c3ccc3ccc5ccccc5c34)c3cccc4oc5ccccc5c34)cc2)cc1 ; c1ccc(-c2cccc(N(c3ccc4sc5ccccc5c4c3)c3cc4c5ccccc5sc4c4c3ccc3ccc5ccccc5c34)c2)cc1 ; c1ccc(-c2ccc(N(c3ccc4c(c3)oc3ccccc34)c3cc4c5ccccc5sc4c4c3ccc3ccc5ccccc5c34)cc2)cc1]

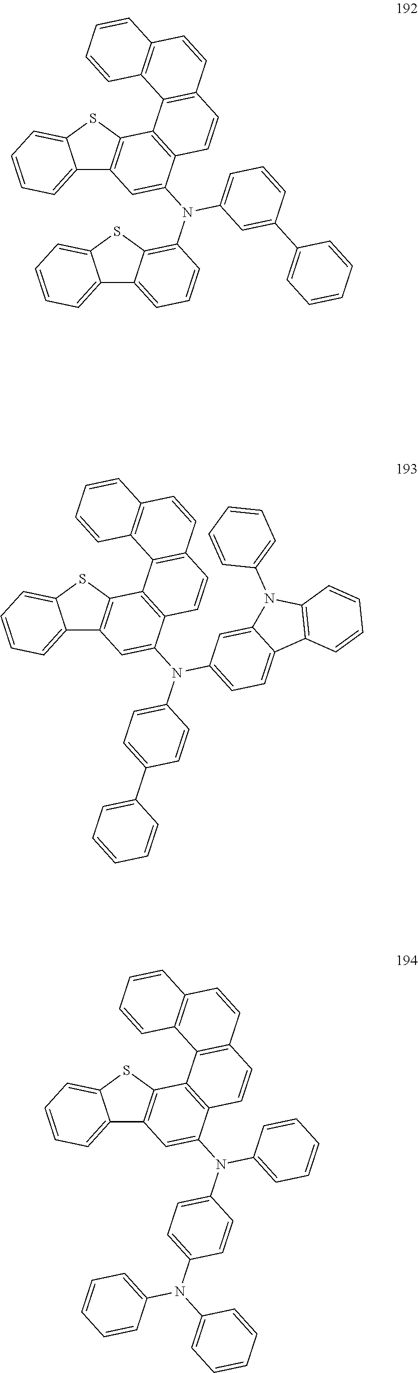 [SMILES: c1ccc(-c2cccc(N(c3cc4c5ccccc5sc4c4c3ccc3ccc5ccccc5c34)c3cccc4c3sc3ccccc34)c2)cc1 ; c1ccc(-c2ccc(N(c3ccc4c5ccccc5n(-c5ccccc5)c4c3)c3cc4c5ccccc5sc4c4c3ccc3ccc5ccccc5c34)cc2)cc1 ; c1ccc(N(c2ccccc2)c2ccc(N(c3ccccc3)c3cc4c5ccccc5sc4c4c3ccc3ccc5ccccc5c34)cc2)cc1]

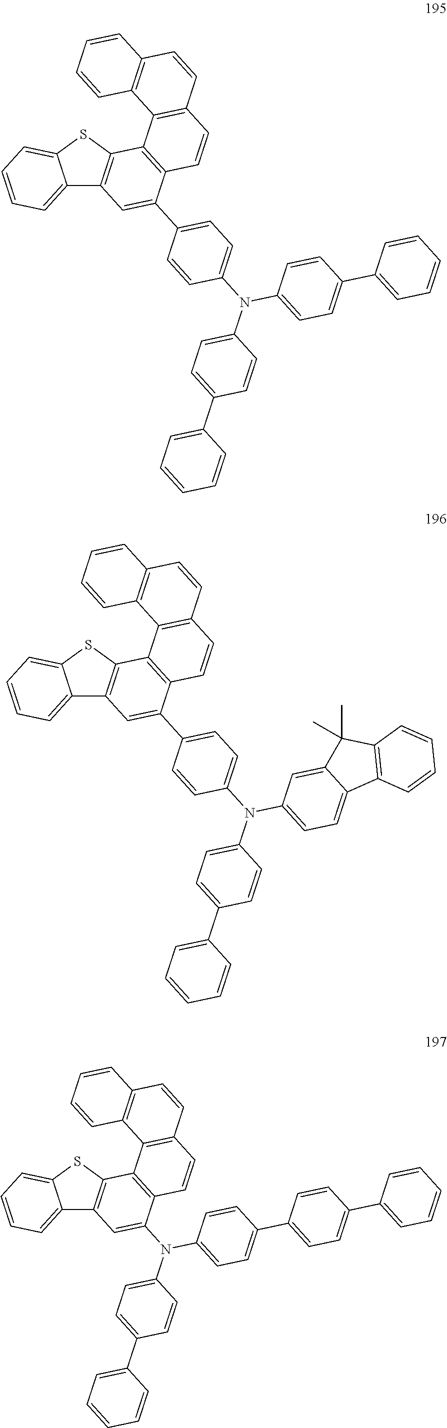 [SMILES: c1ccc(-c2ccc(N(c3ccc(-c4ccccc4)cc3)c3ccc(-c4cc5c6ccccc6sc5c5c4ccc4ccc6ccccc6c45)cc3)cc2)cc1 ; CC1(C)c2ccccc2-c2ccc(N(c3ccc(-c4ccccc4)cc3)c3ccc(-c4cc5c6ccccc6sc5c5c4ccc4ccc6ccccc6c45)cc3)cc21 ; c1ccc(-c2ccc(-c3ccc(N(c4ccc(-c5ccccc5)cc4)c4cc5c6ccccc6sc5c5c4ccc4ccc6ccccc6c45)cc3)cc2)cc1]

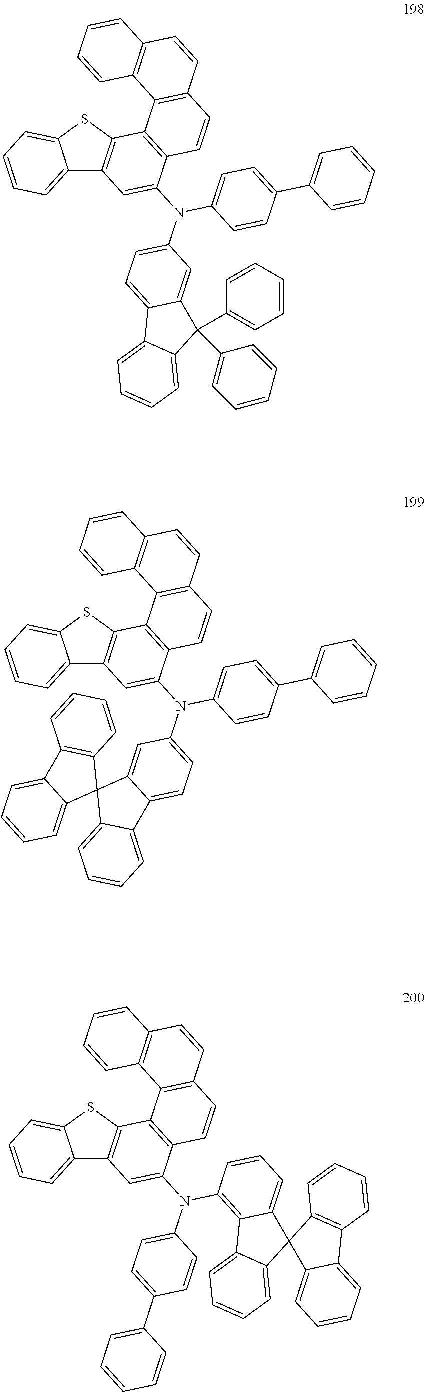 [SMILES: c1ccc(-c2ccc(N(c3ccc4c(c3)C(c3ccccc3)(c3ccccc3)c3ccccc3-4)c3cc4c5ccccc5sc4c4c3ccc3ccc5ccccc5c34)cc2)cc1 ; c1ccc(-c2ccc(N(c3ccc4c(c3)C3(c5ccccc5-c5ccccc53)c3ccccc3-4)c3cc4c5ccccc5sc4c4c3ccc3ccc5ccccc5c34)cc2)cc1 ; c1ccc(-c2ccc(N(c3cccc4c3-c3ccccc3C43c4ccccc4-c4ccccc43)c3cc4c5ccccc5sc4c4c3ccc3ccc5ccccc5c34)cc2)cc1]

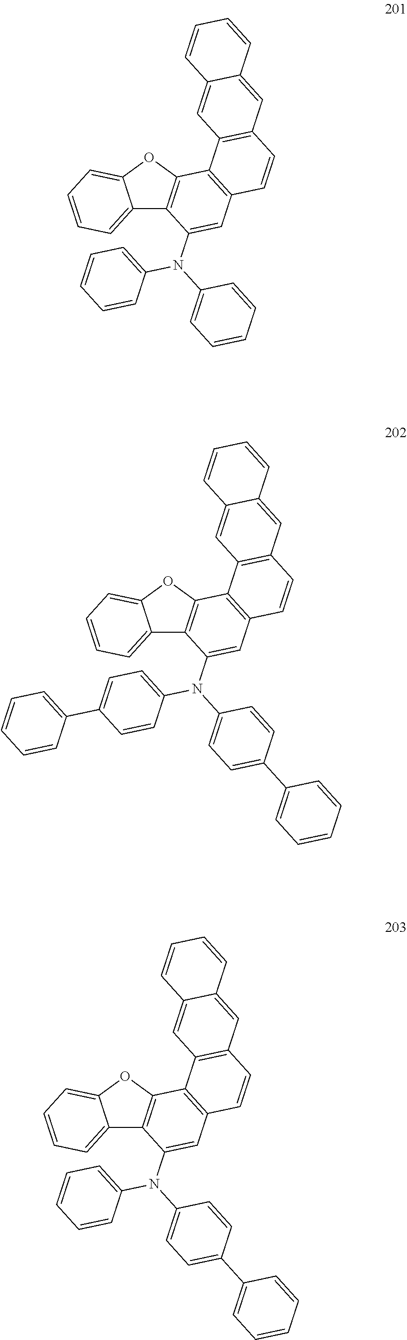 [SMILES: c1ccc(N(c2ccccc2)c2cc3ccc4cc5ccccc5cc4c3c3oc4ccccc4c23)cc1 ; c1ccc(-c2ccc(N(c3ccc(-c4ccccc4)cc3)c3cc4ccc5cc6ccccc6cc5c4c4oc5ccccc5c34)cc2)cc1 ; c1ccc(-c2ccc(N(c3ccccc3)c3cc4ccc5cc6ccccc6cc5c4c4oc5ccccc5c34)cc2)cc1]

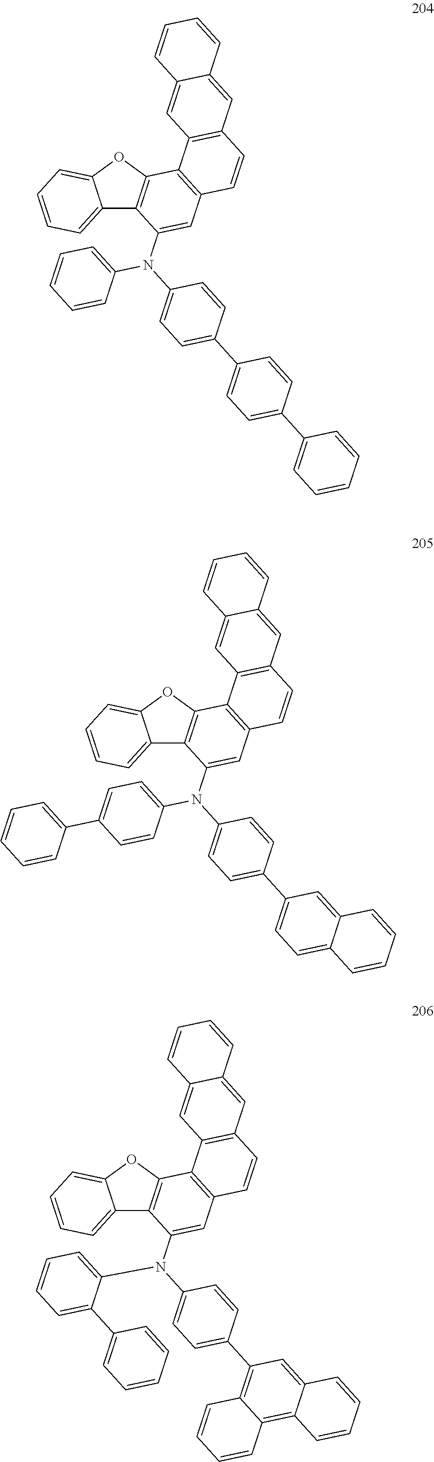 [SMILES: c1ccc(-c2ccc(-c3ccc(N(c4ccccc4)c4cc5ccc6cc7ccccc7cc6c5c5oc6ccccc6c45)cc3)cc2)cc1 ; c1ccc(-c2ccc(N(c3ccc(-c4ccc5ccccc5c4)cc3)c3cc4ccc5cc6ccccc6cc5c4c4oc5ccccc5c34)cc2)cc1 ; c1ccc(-c2ccccc2N(c2ccc(-c3cc4ccccc4c4ccccc34)cc2)c2cc3ccc4cc5ccccc5cc4c3c3oc4ccccc4c23)cc1]

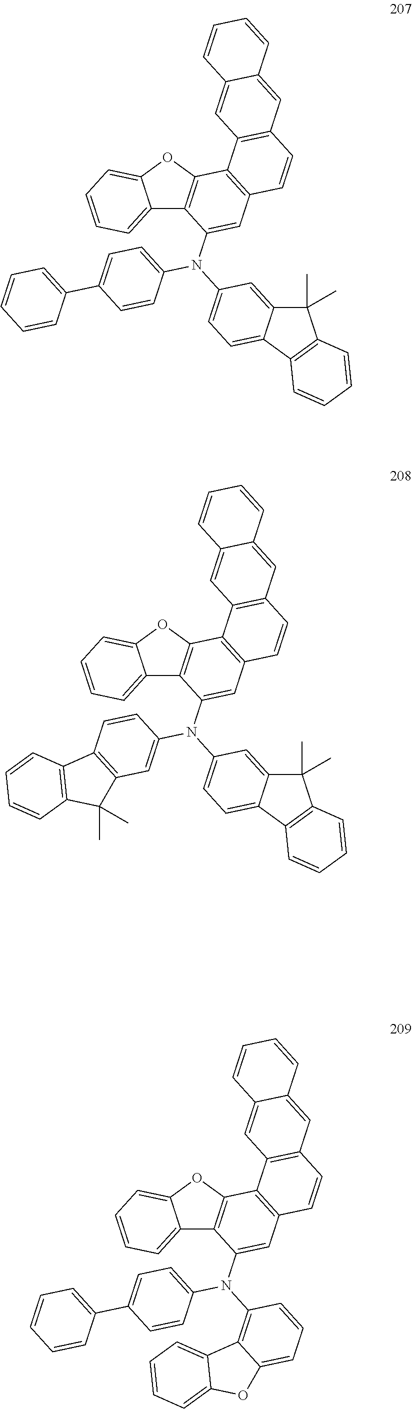 [SMILES: CC1(C)c2ccccc2-c2ccc(N(c3ccc(-c4ccccc4)cc3)c3cc4ccc5cc6ccccc6cc5c4c4oc5ccccc5c34)cc21 ; CC1(C)c2ccccc2-c2ccc(N(c3ccc4c(c3)C(C)(C)c3ccccc3-4)c3cc4ccc5cc6ccccc6cc5c4c4oc5ccccc5c34)cc21 ; c1ccc(-c2ccc(N(c3cccc4oc5ccccc5c34)c3cc4ccc5cc6ccccc6cc5c4c4oc5ccccc5c34)cc2)cc1]

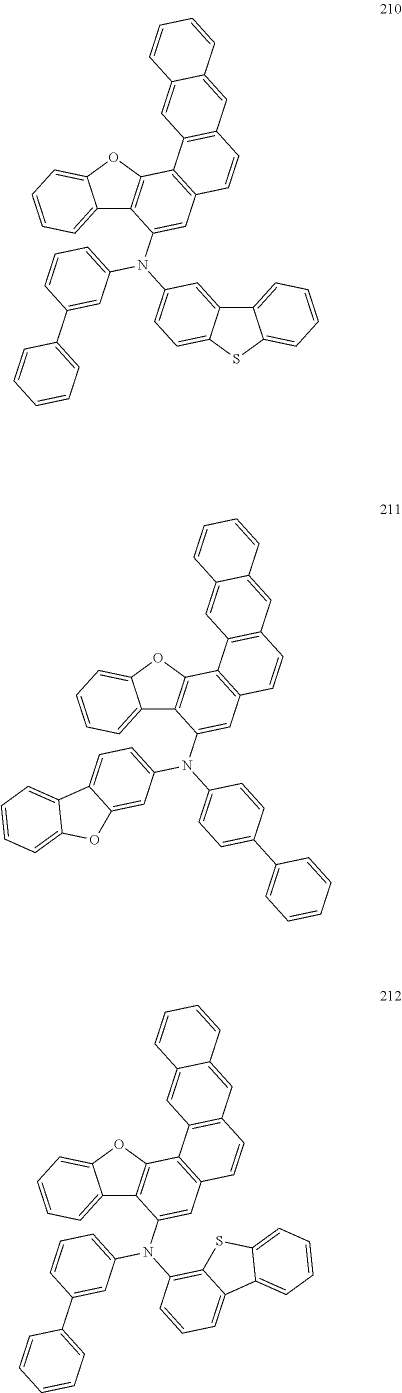 [SMILES: c1ccc(-c2cccc(N(c3ccc4sc5ccccc5c4c3)c3cc4ccc5cc6ccccc6cc5c4c4oc5ccccc5c34)c2)cc1 ; c1ccc(-c2ccc(N(c3ccc4c(c3)oc3ccccc34)c3cc4ccc5cc6ccccc6cc5c4c4oc5ccccc5c34)cc2)cc1 ; c1ccc(-c2cccc(N(c3cccc4c3sc3ccccc34)c3cc4ccc5cc6ccccc6cc5c4c4oc5ccccc5c34)c2)cc1]

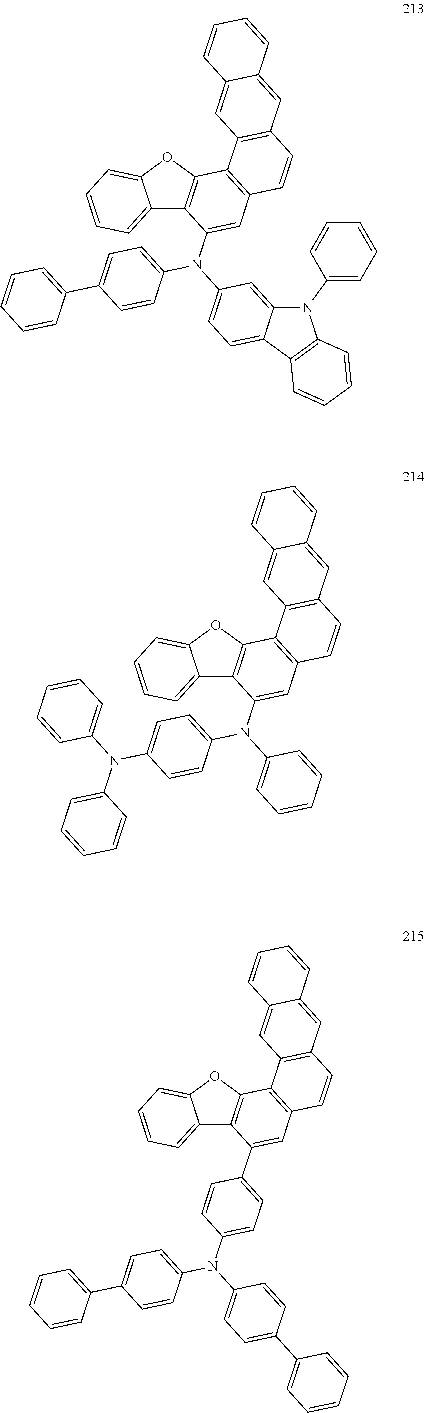 [SMILES: c1ccc(-c2ccc(N(c3ccc4c5ccccc5n(-c5ccccc5)c4c3)c3cc4ccc5cc6ccccc6cc5c4c4oc5ccccc5c34)cc2)cc1 ; c1ccc(N(c2ccccc2)c2ccc(N(c3ccccc3)c3cc4ccc5cc6ccccc6cc5c4c4oc5ccccc5c34)cc2)cc1 ; c1ccc(-c2ccc(N(c3ccc(-c4ccccc4)cc3)c3ccc(-c4cc5ccc6cc7ccccc7cc6c5c5oc6ccccc6c45)cc3)cc2)cc1]

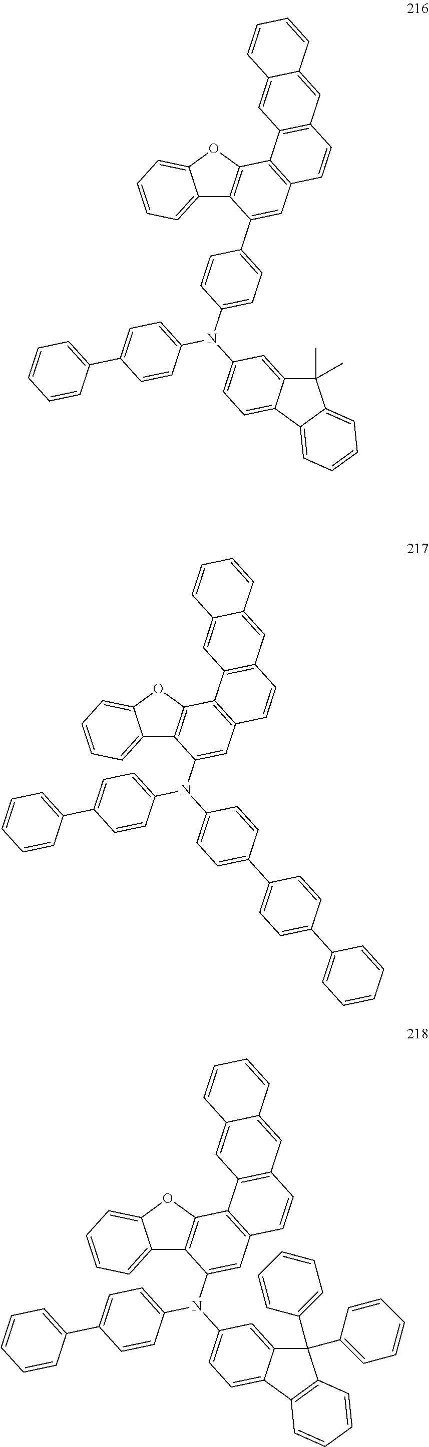 [SMILES: CC1(C)c2ccccc2-c2ccc(N(c3ccc(-c4ccccc4)cc3)c3ccc(-c4cc5ccc6cc7ccccc7cc6c5c5oc6ccccc6c45)cc3)cc21 ; c1ccc(-c2ccc(-c3ccc(N(c4ccc(-c5ccccc5)cc4)c4cc5ccc6cc7ccccc7cc6c5c5oc6ccccc6c45)cc3)cc2)cc1 ; c1ccc(-c2ccc(N(c3ccc4c(c3)C(c3ccccc3)(c3ccccc3)c3ccccc3-4)c3cc4ccc5cc6ccccc6cc5c4c4oc5ccccc5c34)cc2)cc1]

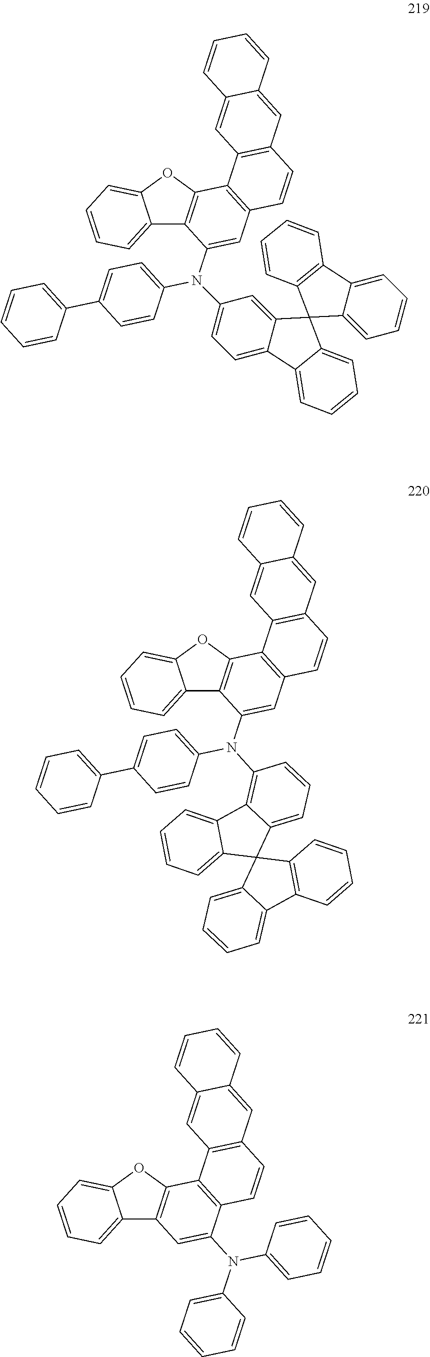 [SMILES: c1ccc(-c2ccc(N(c3ccc4c(c3)C3(c5ccccc5-c5ccccc53)c3ccccc3-4)c3cc4ccc5cc6ccccc6cc5c4c4oc5ccccc5c34)cc2)cc1 ; c1ccc(-c2ccc(N(c3cccc4c3-c3ccccc3C43c4ccccc4-c4ccccc43)c3cc4ccc5cc6ccccc6cc5c4c4oc5ccccc5c34)cc2)cc1 ; c1ccc(N(c2ccccc2)c2cc3c4ccccc4oc3c3c2ccc2cc4ccccc4cc23)cc1]

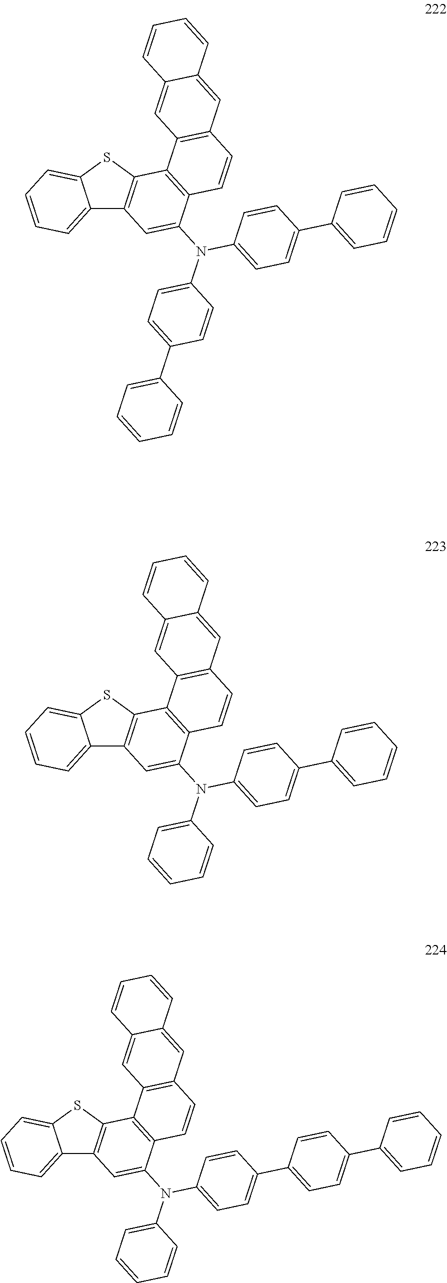 [SMILES: c1ccc(-c2ccc(N(c3ccc(-c4ccccc4)cc3)c3cc4c5ccccc5sc4c4c3ccc3cc5ccccc5cc34)cc2)cc1 ; c1ccc(-c2ccc(N(c3ccccc3)c3cc4c5ccccc5sc4c4c3ccc3cc5ccccc5cc34)cc2)cc1 ; c1ccc(-c2ccc(-c3ccc(N(c4ccccc4)c4cc5c6ccccc6sc5c5c4ccc4cc6ccccc6cc45)cc3)cc2)cc1]

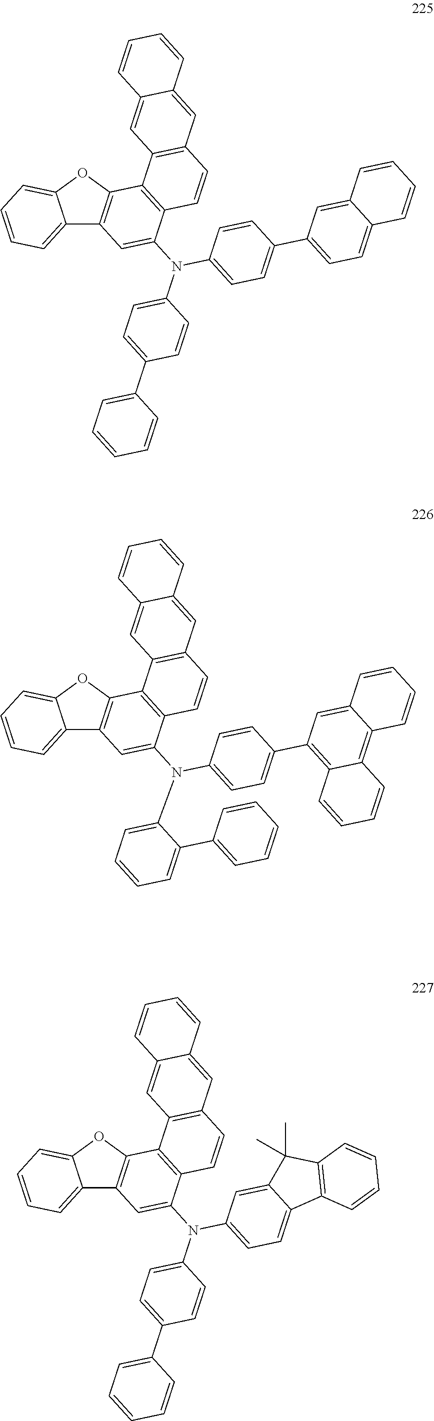 [SMILES: c1ccc(-c2ccc(N(c3ccc(-c4ccc5ccccc5c4)cc3)c3cc4c5ccccc5oc4c4c3ccc3cc5ccccc5cc34)cc2)cc1 ; c1ccc(-c2ccccc2N(c2ccc(-c3cc4ccccc4c4ccccc34)cc2)c2cc3c4ccccc4oc3c3c2ccc2cc4ccccc4cc23)cc1 ; CC1(C)c2ccccc2-c2ccc(N(c3ccc(-c4ccccc4)cc3)c3cc4c5ccccc5oc4c4c3ccc3cc5ccccc5cc34)cc21]

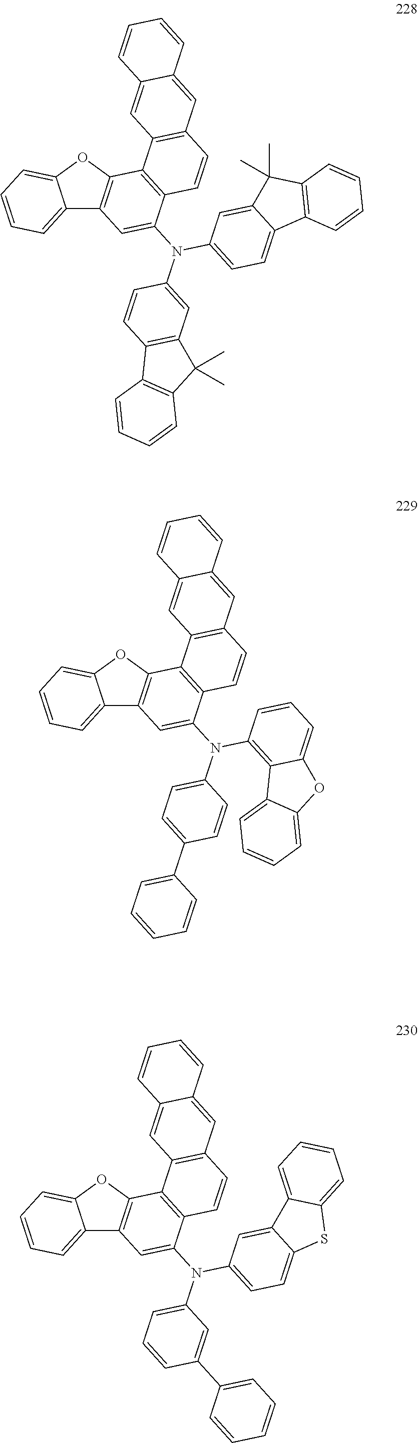 [SMILES: CC1(C)c2ccccc2-c2ccc(N(c3ccc4c(c3)C(C)(C)c3ccccc3-4)c3cc4c5ccccc5oc4c4c3ccc3cc5ccccc5cc34)cc21 ; c1ccc(-c2ccc(N(c3cc4c5ccccc5oc4c4c3ccc3cc5ccccc5cc34)c3cccc4oc5ccccc5c34)cc2)cc1 ; c1ccc(-c2cccc(N(c3ccc4sc5ccccc5c4c3)c3cc4c5ccccc5oc4c4c3ccc3cc5ccccc5cc34)c2)cc1]

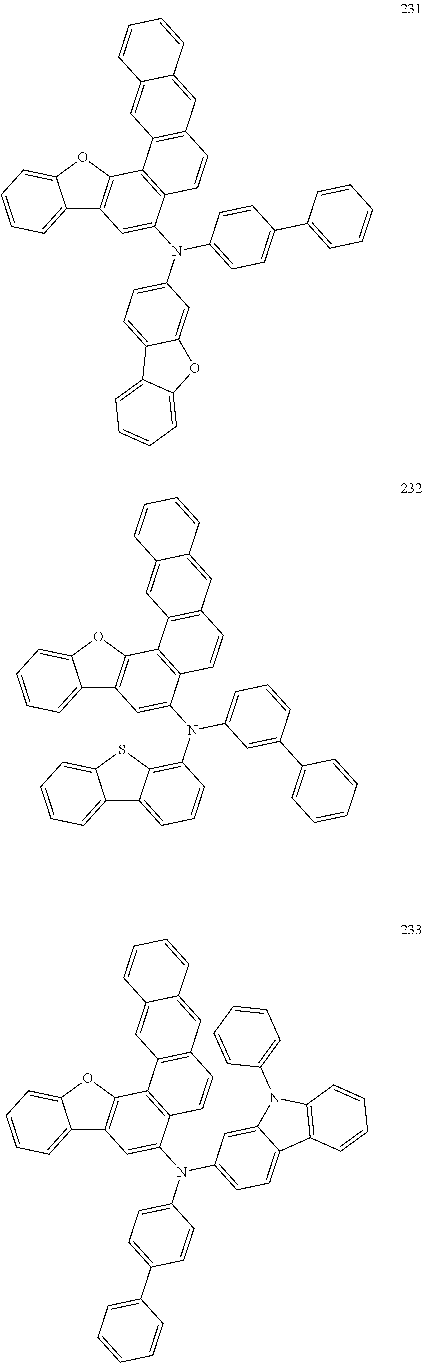 [SMILES: c1ccc(-c2ccc(N(c3ccc4c(c3)oc3ccccc34)c3cc4c5ccccc5oc4c4c3ccc3cc5ccccc5cc34)cc2)cc1 ; c1ccc(-c2cccc(N(c3cc4c5ccccc5oc4c4c3ccc3cc5ccccc5cc34)c3cccc4c3sc3ccccc34)c2)cc1 ; c1ccc(-c2ccc(N(c3ccc4c5ccccc5n(-c5ccccc5)c4c3)c3cc4c5ccccc5oc4c4c3ccc3cc5ccccc5cc34)cc2)cc1]

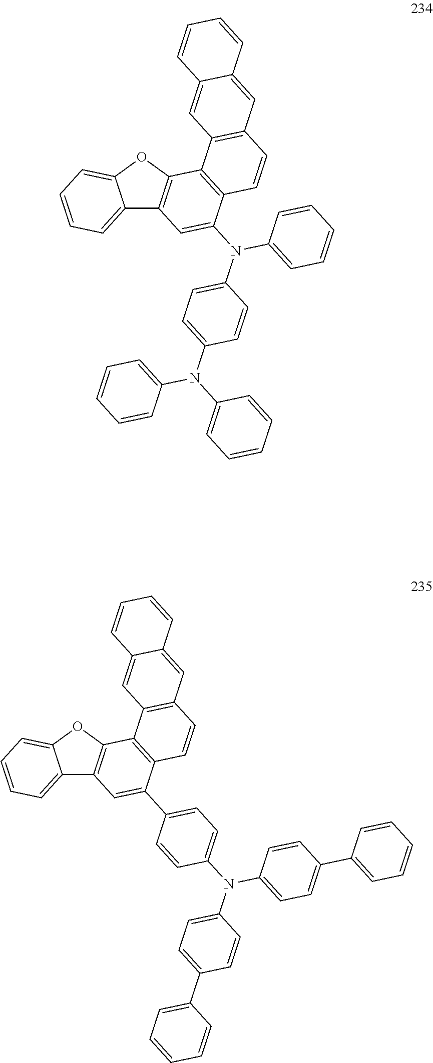 [SMILES: c1ccc(N(c2ccccc2)c2ccc(N(c3ccccc3)c3cc4c5ccccc5oc4c4c3ccc3cc5ccccc5cc34)cc2)cc1 ; c1ccc(-c2ccc(N(c3ccc(-c4ccccc4)cc3)c3ccc(-c4cc5c6ccccc6oc5c5c4ccc4cc6ccccc6cc45)cc3)cc2)cc1]

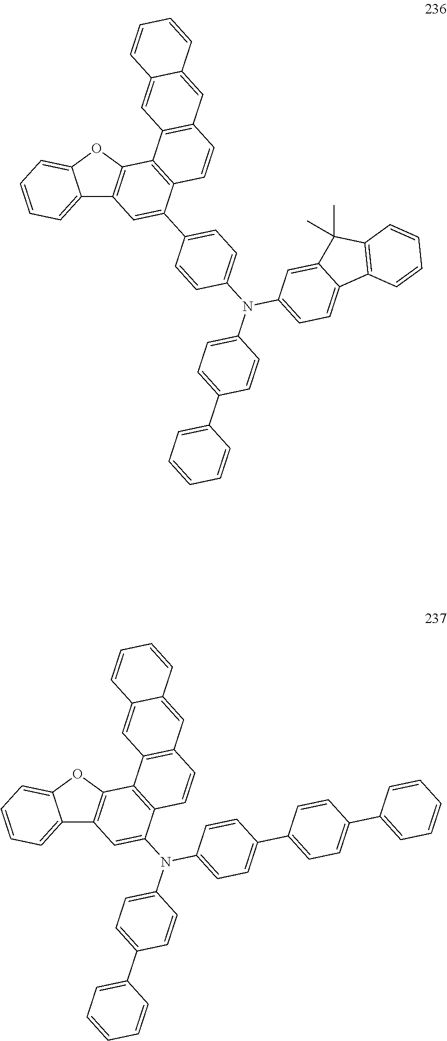 [SMILES: CC1(C)c2ccccc2-c2ccc(N(c3ccc(-c4ccccc4)cc3)c3ccc(-c4cc5c6ccccc6oc5c5c4ccc4cc6ccccc6cc45)cc3)cc21 ; c1ccc(-c2ccc(-c3ccc(N(c4ccc(-c5ccccc5)cc4)c4cc5c6ccccc6oc5c5c4ccc4cc6ccccc6cc45)cc3)cc2)cc1]

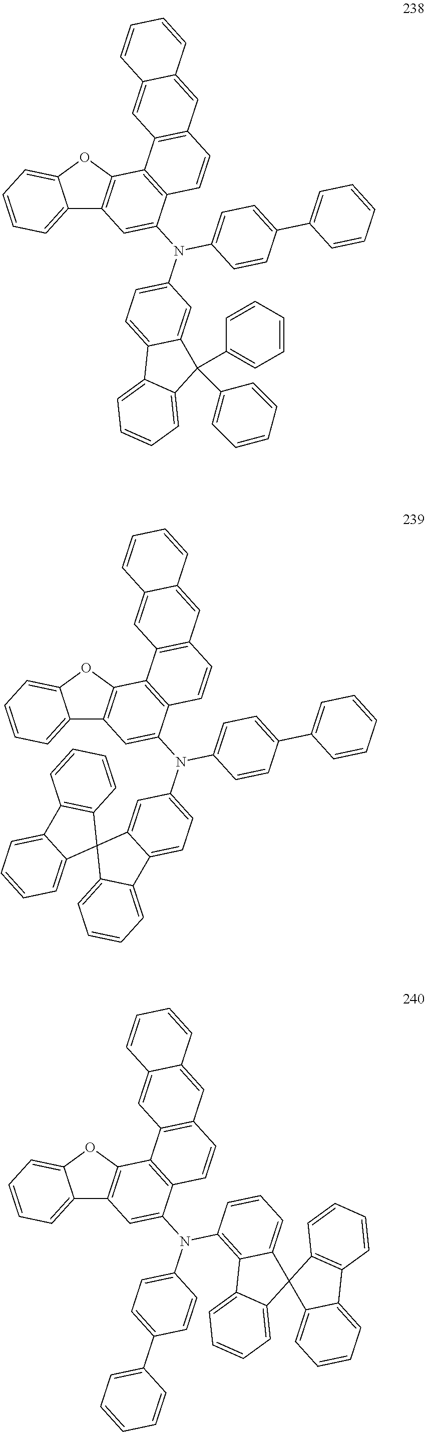 [SMILES: c1ccc(-c2ccc(N(c3ccc4c(c3)C(c3ccccc3)(c3ccccc3)c3ccccc3-4)c3cc4c5ccccc5oc4c4c3ccc3cc5ccccc5cc34)cc2)cc1 ; c1ccc(-c2ccc(N(c3ccc4c(c3)C3(c5ccccc5-c5ccccc53)c3ccccc3-4)c3cc4c5ccccc5oc4c4c3ccc3cc5ccccc5cc34)cc2)cc1 ; c1ccc(-c2ccc(N(c3cccc4c3-c3ccccc3C43c4ccccc4-c4ccccc43)c3cc4c5ccccc5oc4c4c3ccc3cc5ccccc5cc34)cc2)cc1]

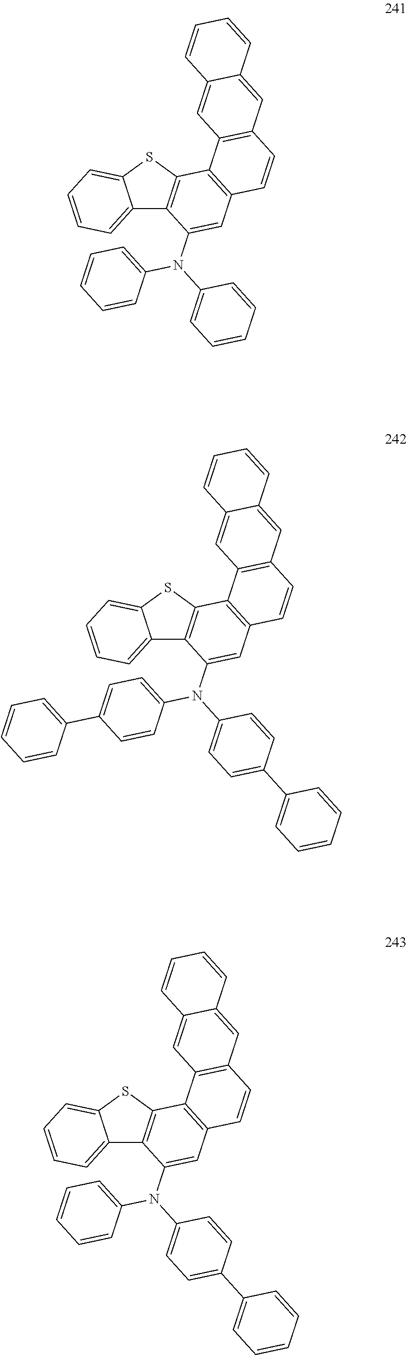 [SMILES: c1ccc(N(c2ccccc2)c2cc3ccc4cc5ccccc5cc4c3c3sc4ccccc4c23)cc1 ; c1ccc(-c2ccc(N(c3ccc(-c4ccccc4)cc3)c3cc4ccc5cc6ccccc6cc5c4c4sc5ccccc5c34)cc2)cc1 ; c1ccc(-c2ccc(N(c3ccccc3)c3cc4ccc5cc6ccccc6cc5c4c4sc5ccccc5c34)cc2)cc1]

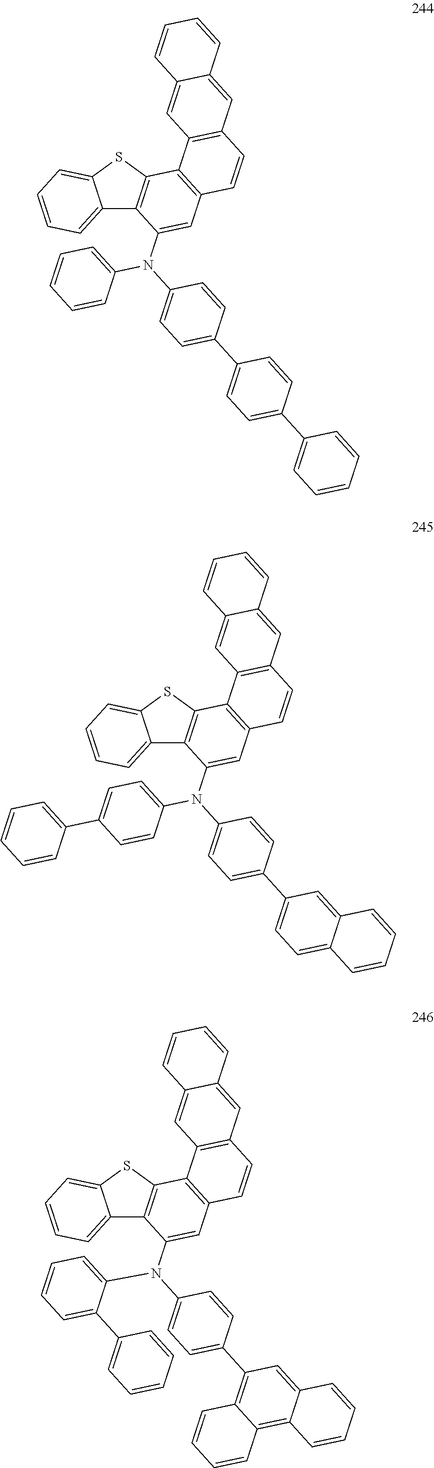 [SMILES: c1ccc(-c2ccc(-c3ccc(N(c4ccccc4)c4cc5ccc6cc7ccccc7cc6c5c5sc6ccccc6c45)cc3)cc2)cc1 ; c1ccc(-c2ccc(N(c3ccc(-c4ccc5ccccc5c4)cc3)c3cc4ccc5cc6ccccc6cc5c4c4sc5ccccc5c34)cc2)cc1 ; c1ccc(-c2ccccc2N(c2ccc(-c3cc4ccccc4c4ccccc34)cc2)c2cc3ccc4cc5ccccc5cc4c3c3sc4ccccc4c23)cc1]

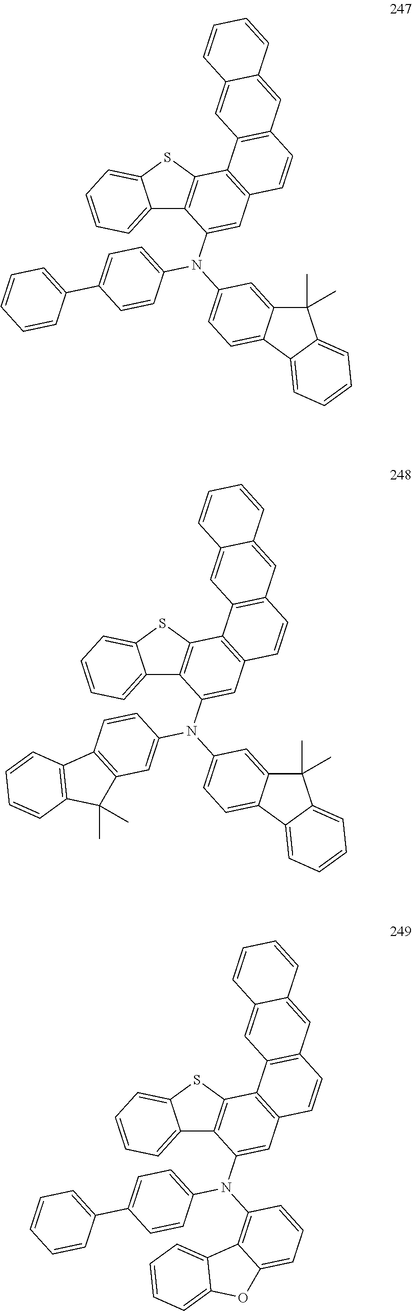 [SMILES: CC1(C)c2ccccc2-c2ccc(N(c3ccc(-c4ccccc4)cc3)c3cc4ccc5cc6ccccc6cc5c4c4sc5ccccc5c34)cc21 ; CC1(C)c2ccccc2-c2ccc(N(c3ccc4c(c3)C(C)(C)c3ccccc3-4)c3cc4ccc5cc6ccccc6cc5c4c4sc5ccccc5c34)cc21 ; c1ccc(-c2ccc(N(c3cccc4oc5ccccc5c34)c3cc4ccc5cc6ccccc6cc5c4c4sc5ccccc5c34)cc2)cc1]

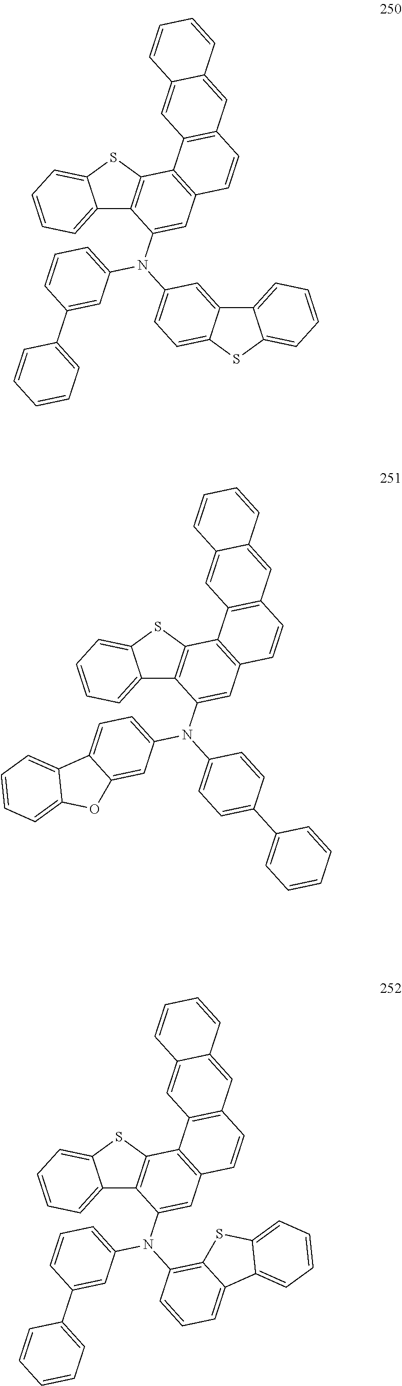 [SMILES: c1ccc(-c2cccc(N(c3ccc4sc5ccccc5c4c3)c3cc4ccc5cc6ccccc6cc5c4c4sc5ccccc5c34)c2)cc1 ; c1ccc(-c2ccc(N(c3ccc4c(c3)oc3ccccc34)c3cc4ccc5cc6ccccc6cc5c4c4sc5ccccc5c34)cc2)cc1 ; c1ccc(-c2cccc(N(c3cccc4c3sc3ccccc34)c3cc4ccc5cc6ccccc6cc5c4c4sc5ccccc5c34)c2)cc1]

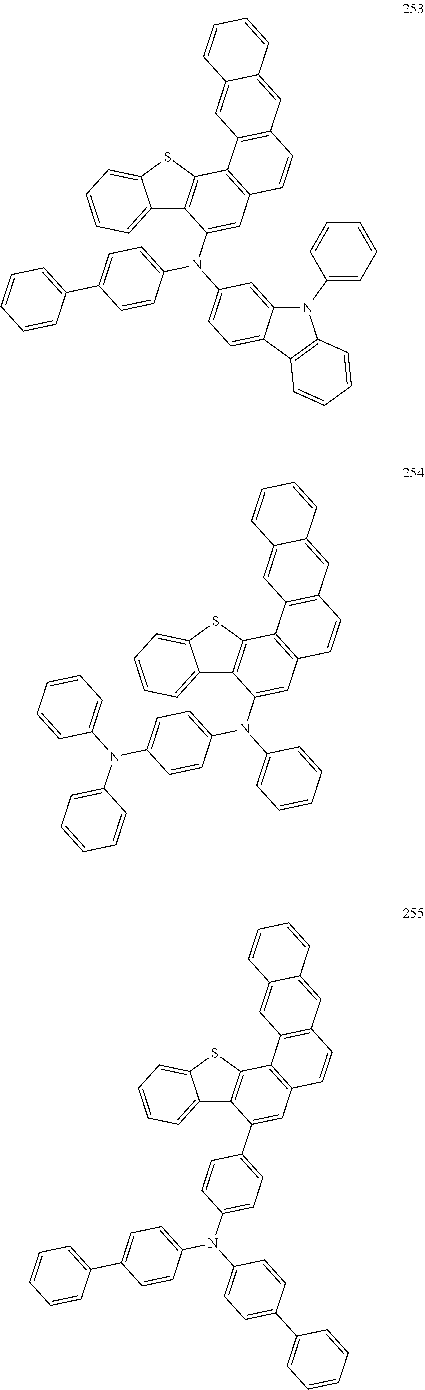 [SMILES: c1ccc(-c2ccc(N(c3ccc4c5ccccc5n(-c5ccccc5)c4c3)c3cc4ccc5cc6ccccc6cc5c4c4sc5ccccc5c34)cc2)cc1 ; c1ccc(N(c2ccccc2)c2ccc(N(c3ccccc3)c3cc4ccc5cc6ccccc6cc5c4c4sc5ccccc5c34)cc2)cc1 ; c1ccc(-c2ccc(N(c3ccc(-c4ccccc4)cc3)c3ccc(-c4cc5ccc6cc7ccccc7cc6c5c5sc6ccccc6c45)cc3)cc2)cc1]

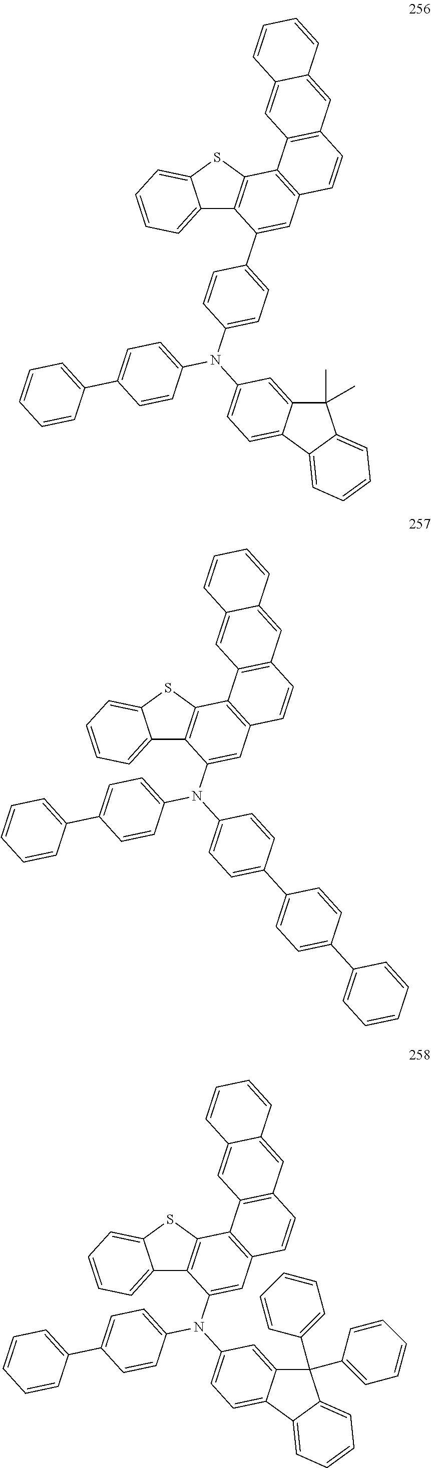 [SMILES: CC1(C)c2ccccc2-c2ccc(N(c3ccc(-c4ccccc4)cc3)c3ccc(-c4cc5ccc6cc7ccccc7cc6c5c5sc6ccccc6c45)cc3)cc21 ; c1ccc(-c2ccc(-c3ccc(N(c4ccc(-c5ccccc5)cc4)c4cc5ccc6cc7ccccc7cc6c5c5sc6ccccc6c45)cc3)cc2)cc1 ; c1ccc(-c2ccc(N(c3ccc4c(c3)C(c3ccccc3)(c3ccccc3)c3ccccc3-4)c3cc4ccc5cc6ccccc6cc5c4c4sc5ccccc5c34)cc2)cc1]

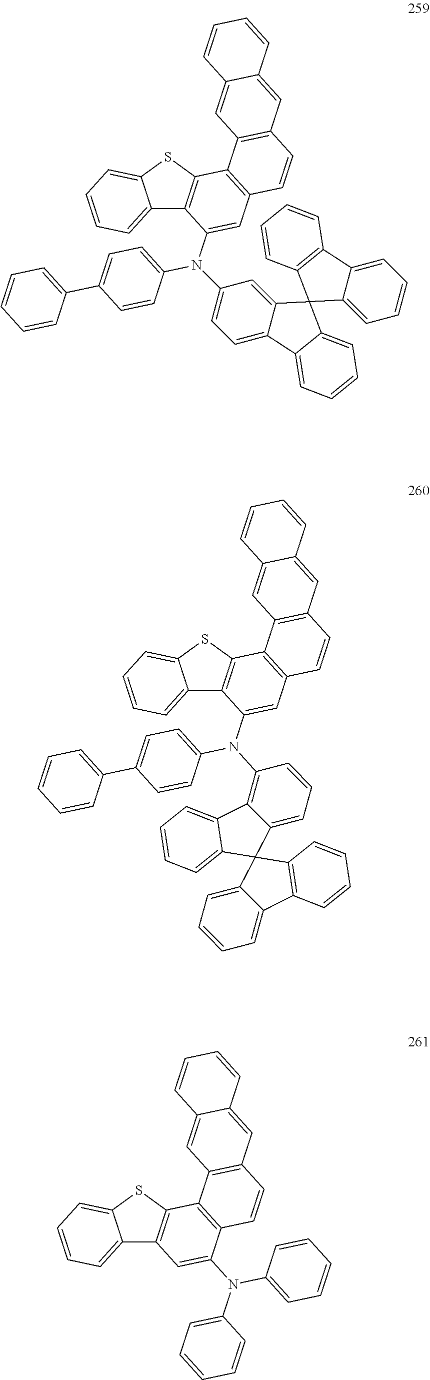 [SMILES: c1ccc(-c2ccc(N(c3ccc4c(c3)C3(c5ccccc5-c5ccccc53)c3ccccc3-4)c3cc4ccc5cc6ccccc6cc5c4c4sc5ccccc5c34)cc2)cc1 ; c1ccc(-c2ccc(N(c3cccc4c3-c3ccccc3C43c4ccccc4-c4ccccc43)c3cc4ccc5cc6ccccc6cc5c4c4sc5ccccc5c34)cc2)cc1 ; c1ccc(N(c2ccccc2)c2cc3c4ccccc4sc3c3c2ccc2cc4ccccc4cc23)cc1]

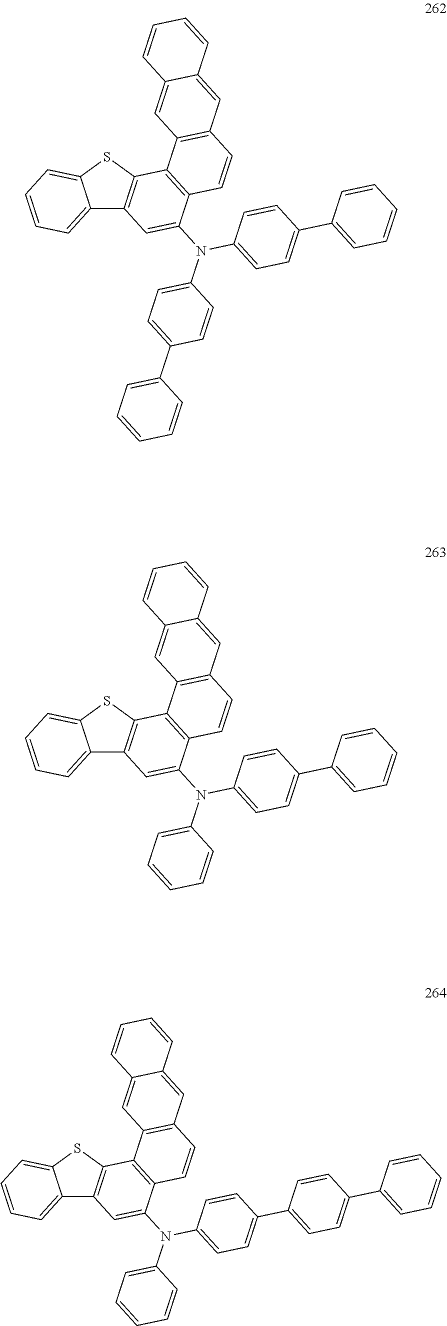 [SMILES: c1ccc(-c2ccc(N(c3ccc(-c4ccccc4)cc3)c3cc4c5ccccc5sc4c4c3ccc3cc5ccccc5cc34)cc2)cc1 ; c1ccc(-c2ccc(N(c3ccccc3)c3cc4c5ccccc5sc4c4c3ccc3cc5ccccc5cc34)cc2)cc1 ; c1ccc(-c2ccc(-c3ccc(N(c4ccccc4)c4cc5c6ccccc6sc5c5c4ccc4cc6ccccc6cc45)cc3)cc2)cc1]

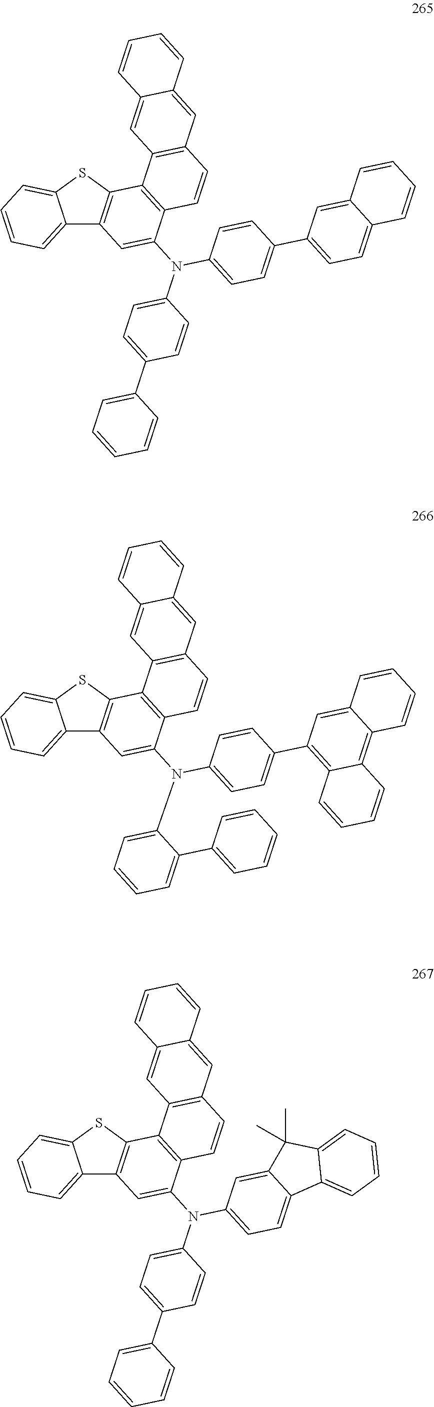 [SMILES: c1ccc(-c2ccc(N(c3ccc(-c4ccc5ccccc5c4)cc3)c3cc4c5ccccc5sc4c4c3ccc3cc5ccccc5cc34)cc2)cc1 ; c1ccc(-c2ccccc2N(c2ccc(-c3cc4ccccc4c4ccccc34)cc2)c2cc3c4ccccc4sc3c3c2ccc2cc4ccccc4cc23)cc1 ; CC1(C)c2ccccc2-c2ccc(N(c3ccc(-c4ccccc4)cc3)c3cc4c5ccccc5sc4c4c3ccc3cc5ccccc5cc34)cc21]

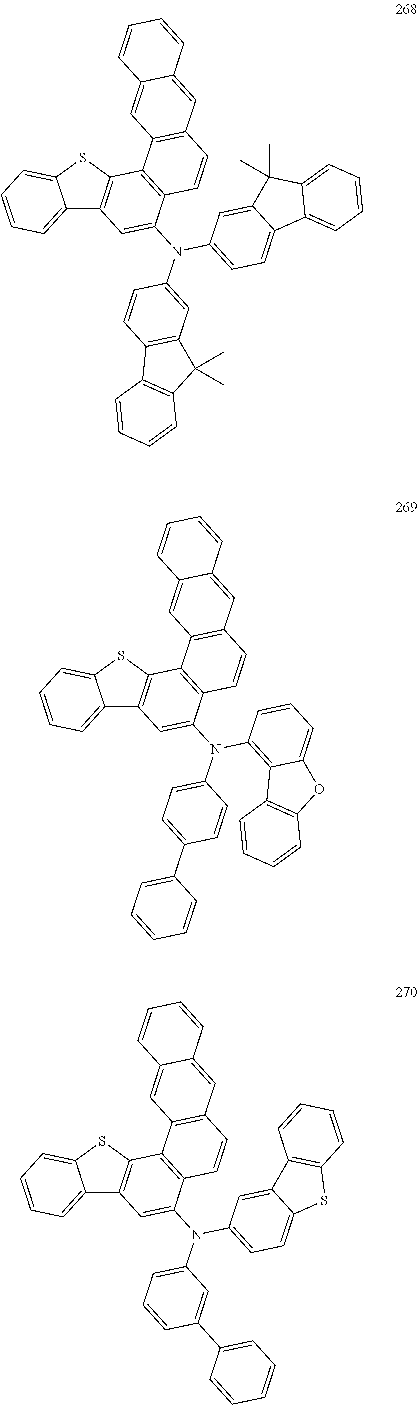 [SMILES: CC1(C)c2ccccc2-c2ccc(N(c3ccc4c(c3)C(C)(C)c3ccccc3-4)c3cc4c5ccccc5sc4c4c3ccc3cc5ccccc5cc34)cc21 ; c1ccc(-c2ccc(N(c3cc4c5ccccc5sc4c4c3ccc3cc5ccccc5cc34)c3cccc4oc5ccccc5c34)cc2)cc1 ; c1ccc(-c2cccc(N(c3ccc4sc5ccccc5c4c3)c3cc4c5ccccc5sc4c4c3ccc3cc5ccccc5cc34)c2)cc1]

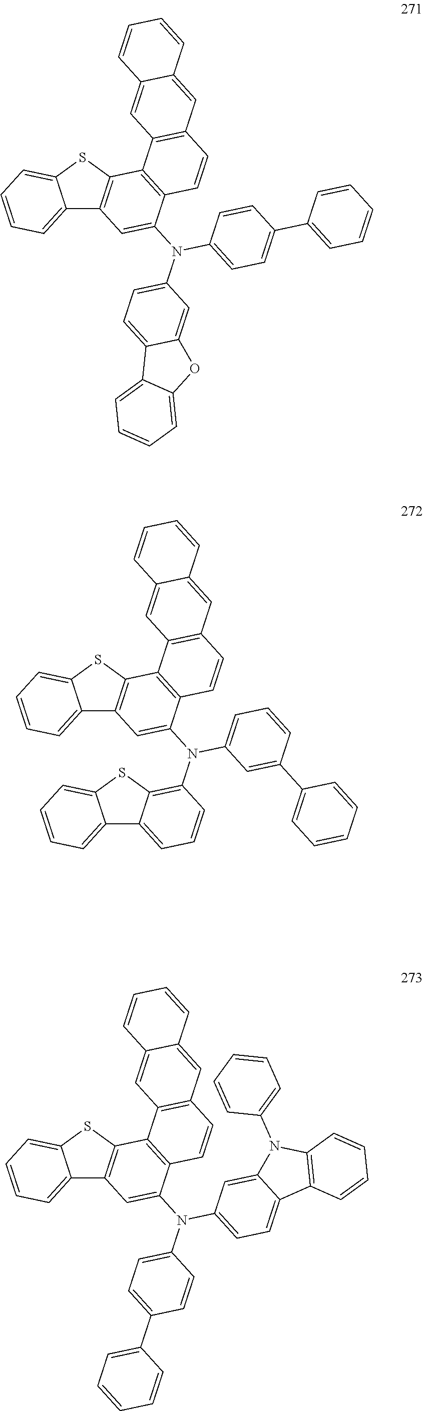 [SMILES: c1ccc(-c2ccc(N(c3ccc4c(c3)oc3ccccc34)c3cc4c5ccccc5sc4c4c3ccc3cc5ccccc5cc34)cc2)cc1 ; c1ccc(-c2cccc(N(c3cc4c5ccccc5sc4c4c3ccc3cc5ccccc5cc34)c3cccc4c3sc3ccccc34)c2)cc1 ; c1ccc(-c2ccc(N(c3ccc4c5ccccc5n(-c5ccccc5)c4c3)c3cc4c5ccccc5sc4c4c3ccc3cc5ccccc5cc34)cc2)cc1]

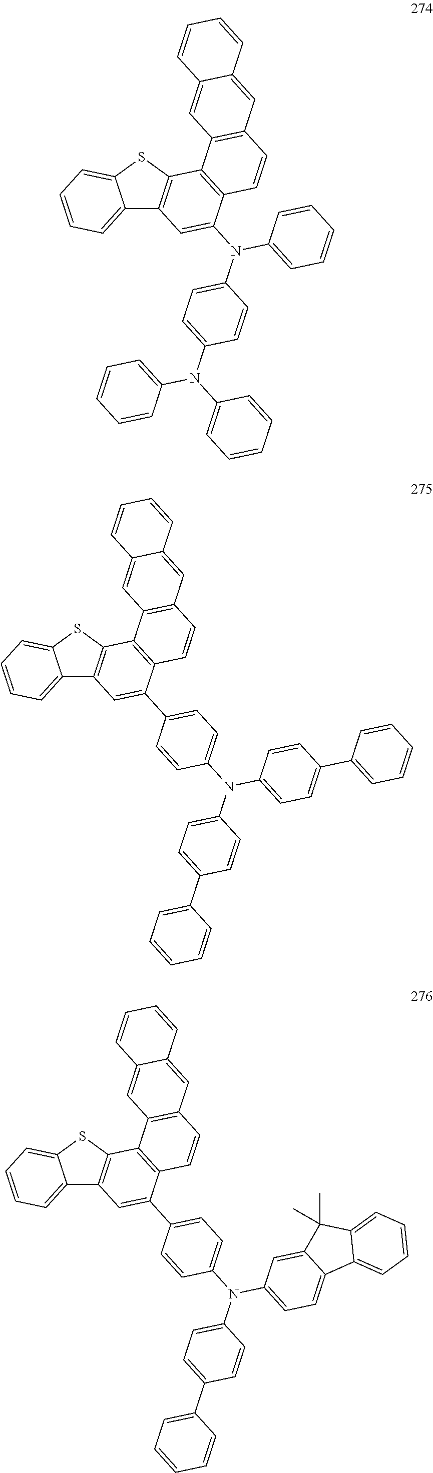

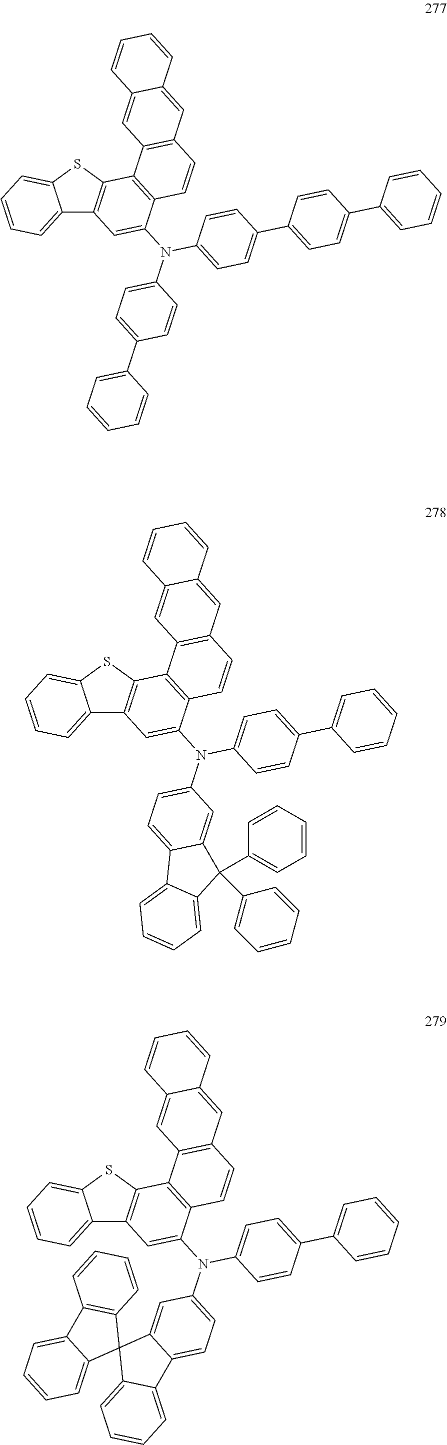 [SMILES: c1ccc(-c2ccc(-c3ccc(N(c4ccc(-c5ccccc5)cc4)c4cc5c6ccccc6sc5c5c4ccc4cc6ccccc6cc45)cc3)cc2)cc1 ; c1ccc(-c2ccc(N(c3ccc4c(c3)C(c3ccccc3)(c3ccccc3)c3ccccc3-4)c3cc4c5ccccc5sc4c4c3ccc3cc5ccccc5cc34)cc2)cc1 ; c1ccc(-c2ccc(N(c3ccc4c(c3)C3(c5ccccc5-c5ccccc53)c3ccccc3-4)c3cc4c5ccccc5sc4c4c3ccc3cc5ccccc5cc34)cc2)cc1]

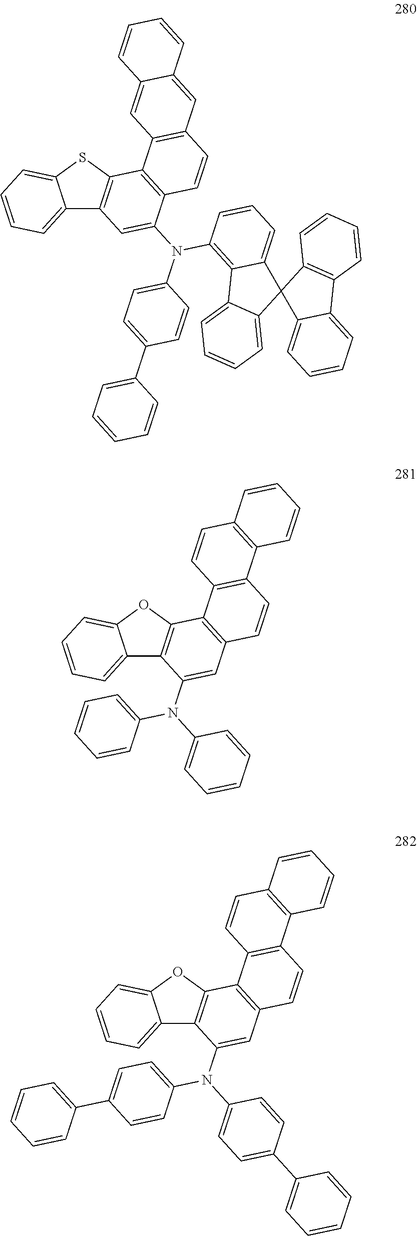 [SMILES: c1ccc(-c2ccc(N(c3cccc4c3-c3ccccc3C43c4ccccc4-c4ccccc43)c3cc4c5ccccc5sc4c4c3ccc3cc5ccccc5cc34)cc2)cc1 ; c1ccc(N(c2ccccc2)c2cc3ccc4c5ccccc5ccc4c3c3oc4ccccc4c23)cc1 ; c1ccc(-c2ccc(N(c3ccc(-c4ccccc4)cc3)c3cc4ccc5c6ccccc6ccc5c4c4oc5ccccc5c34)cc2)cc1]

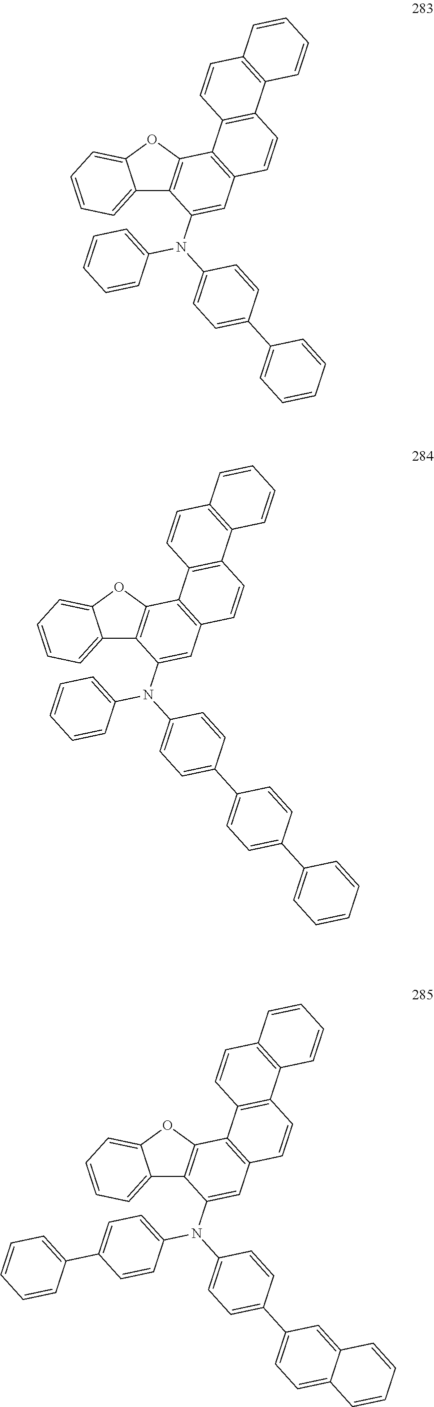 [SMILES: c1ccc(-c2ccc(N(c3ccccc3)c3cc4ccc5c6ccccc6ccc5c4c4oc5ccccc5c34)cc2)cc1 ; c1ccc(-c2ccc(-c3ccc(N(c4ccccc4)c4cc5ccc6c7ccccc7ccc6c5c5oc6ccccc6c45)cc3)cc2)cc1 ; c1ccc(-c2ccc(N(c3ccc(-c4ccc5ccccc5c4)cc3)c3cc4ccc5c6ccccc6ccc5c4c4oc5ccccc5c34)cc2)cc1]

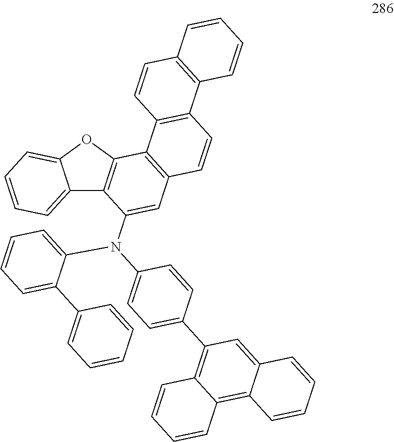 [SMILES: c1ccc(-c2ccccc2N(c2ccc(-c3cc4ccccc4c4ccccc34)cc2)c2cc3ccc4c5ccccc5ccc4c3c3oc4ccccc4c23)cc1]

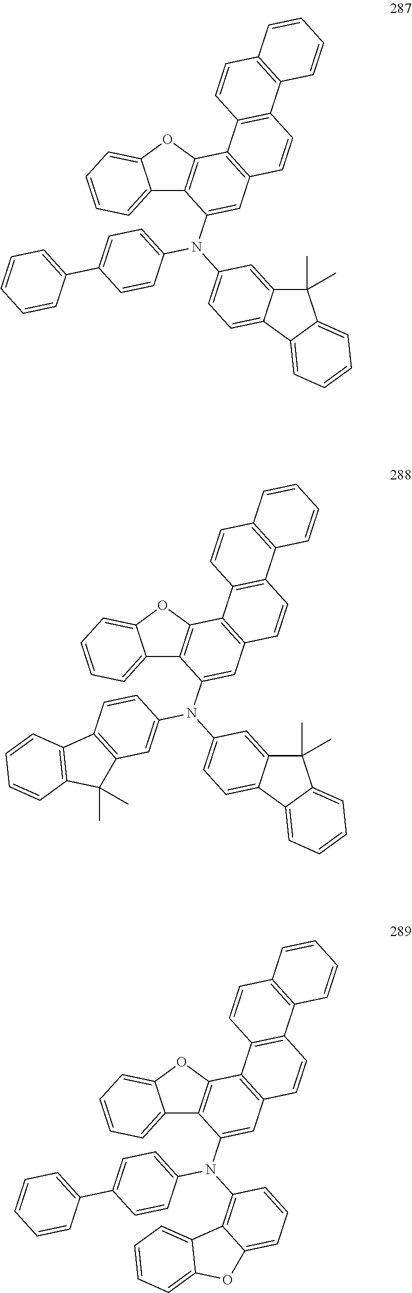 [SMILES: CC1(C)c2ccccc2-c2ccc(N(c3ccc(-c4ccccc4)cc3)c3cc4ccc5c6ccccc6ccc5c4c4oc5ccccc5c34)cc21 ; CC1(C)c2ccccc2-c2ccc(N(c3ccc4c(c3)C(C)(C)c3ccccc3-4)c3cc4ccc5c6ccccc6ccc5c4c4oc5ccccc5c34)cc21 ; c1ccc(-c2ccc(N(c3cccc4oc5ccccc5c34)c3cc4ccc5c6ccccc6ccc5c4c4oc5ccccc5c34)cc2)cc1]

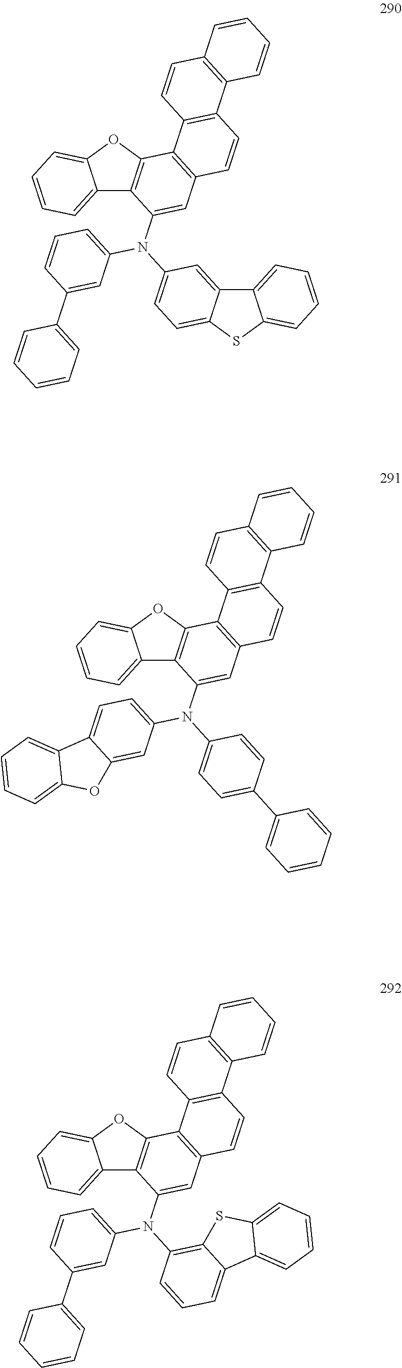 [SMILES: c1ccc(-c2cccc(N(c3ccc4sc5ccccc5c4c3)c3cc4ccc5c6ccccc6ccc5c4c4oc5ccccc5c34)c2)cc1 ; c1ccc(-c2ccc(N(c3ccc4c(c3)oc3ccccc34)c3cc4ccc5c6ccccc6ccc5c4c4oc5ccccc5c34)cc2)cc1 ; c1ccc(-c2cccc(N(c3cccc4c3sc3ccccc34)c3cc4ccc5c6ccccc6ccc5c4c4oc5ccccc5c34)c2)cc1]

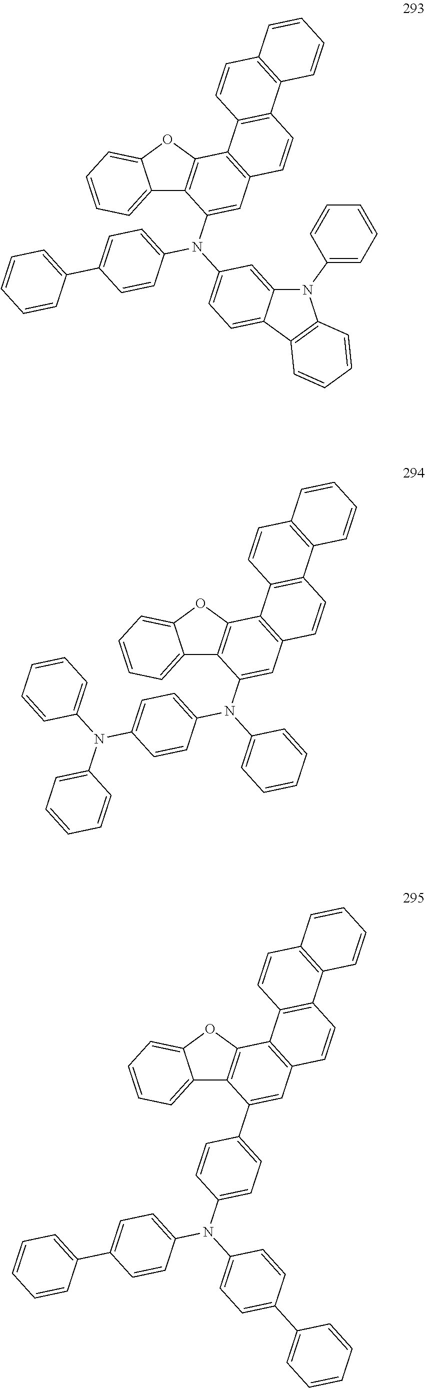 [SMILES: c1ccc(-c2ccc(N(c3ccc4c5ccccc5n(-c5ccccc5)c4c3)c3cc4ccc5c6ccccc6ccc5c4c4oc5ccccc5c34)cc2)cc1 ; c1ccc(N(c2ccccc2)c2ccc(N(c3ccccc3)c3cc4ccc5c6ccccc6ccc5c4c4oc5ccccc5c34)cc2)cc1 ; c1ccc(-c2ccc(N(c3ccc(-c4ccccc4)cc3)c3ccc(-c4cc5ccc6c7ccccc7ccc6c5c5oc6ccccc6c45)cc3)cc2)cc1]

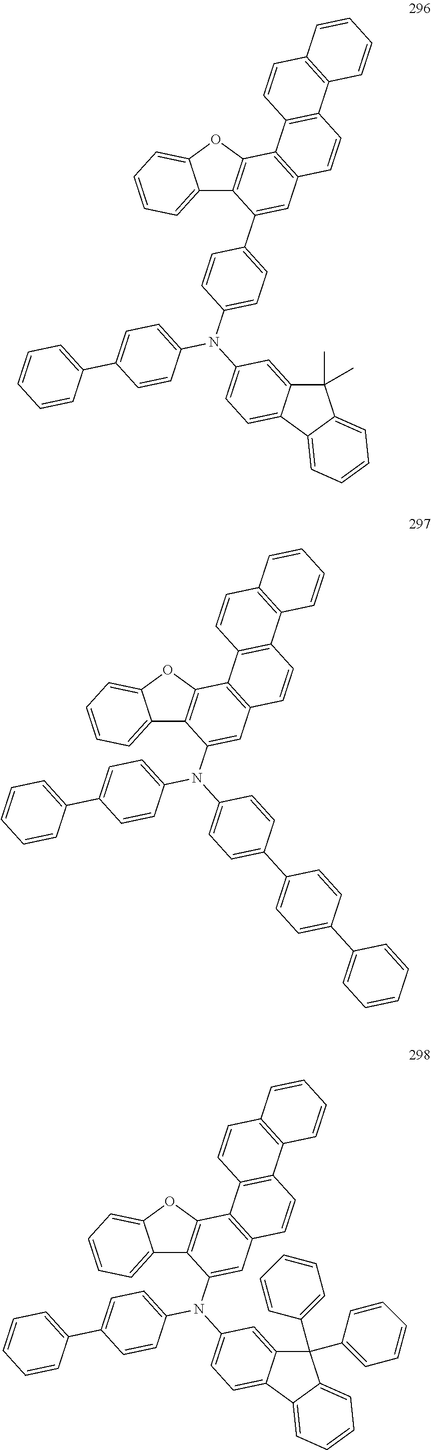 [SMILES: CC1(C)c2ccccc2-c2ccc(N(c3ccc(-c4ccccc4)cc3)c3ccc(-c4cc5ccc6c7ccccc7ccc6c5c5oc6ccccc6c45)cc3)cc21 ; c1ccc(-c2ccc(-c3ccc(N(c4ccc(-c5ccccc5)cc4)c4cc5ccc6c7ccccc7ccc6c5c5oc6ccccc6c45)cc3)cc2)cc1 ; c1ccc(-c2ccc(N(c3ccc4c(c3)C(c3ccccc3)(c3ccccc3)c3ccccc3-4)c3cc4ccc5c6ccccc6ccc5c4c4oc5ccccc5c34)cc2)cc1]

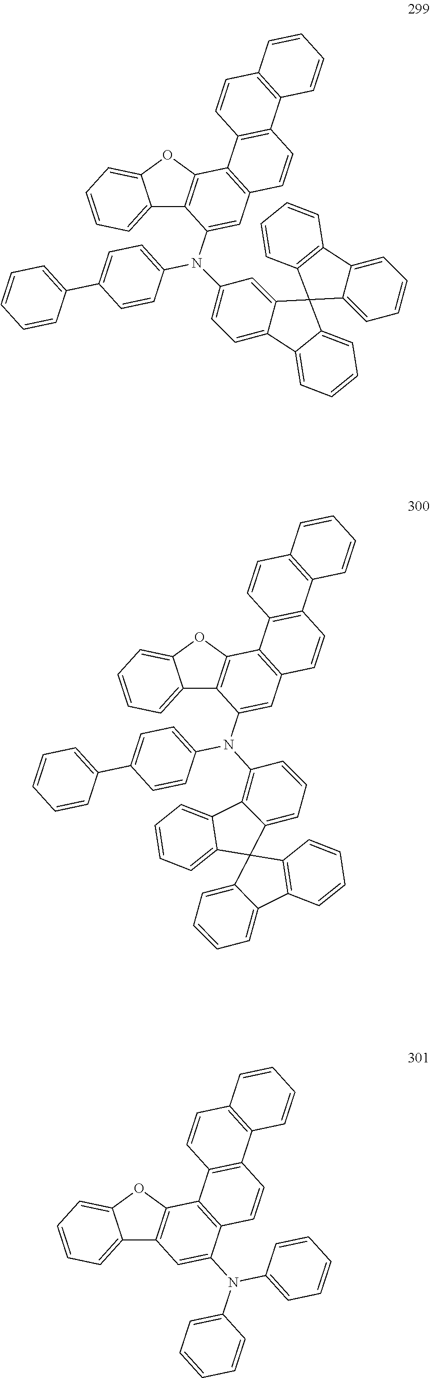 [SMILES: c1ccc(-c2ccc(N(c3ccc4c(c3)C3(c5ccccc5-c5ccccc53)c3ccccc3-4)c3cc4ccc5c6ccccc6ccc5c4c4oc5ccccc5c34)cc2)cc1 ; c1ccc(-c2ccc(N(c3cccc4c3-c3ccccc3C43c4ccccc4-c4ccccc43)c3cc4ccc5c6ccccc6ccc5c4c4oc5ccccc5c34)cc2)cc1 ; c1ccc(N(c2ccccc2)c2cc3c4ccccc4oc3c3c2ccc2c4ccccc4ccc23)cc1]

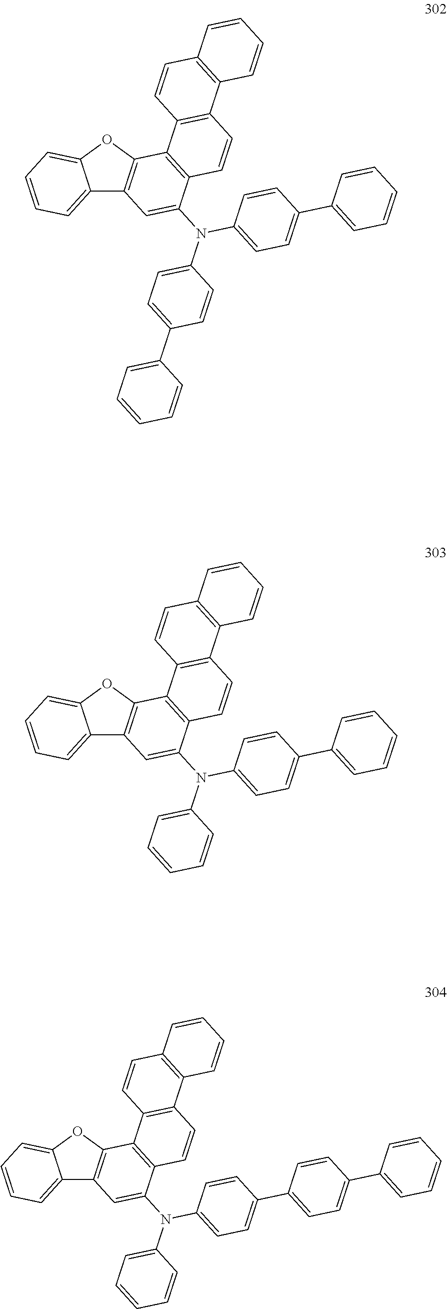 [SMILES: c1ccc(-c2ccc(N(c3ccc(-c4ccccc4)cc3)c3cc4c5ccccc5oc4c4c3ccc3c5ccccc5ccc34)cc2)cc1 ; c1ccc(-c2ccc(N(c3ccccc3)c3cc4c5ccccc5oc4c4c3ccc3c5ccccc5ccc34)cc2)cc1 ; c1ccc(-c2ccc(-c3ccc(N(c4ccccc4)c4cc5c6ccccc6oc5c5c4ccc4c6ccccc6ccc45)cc3)cc2)cc1]

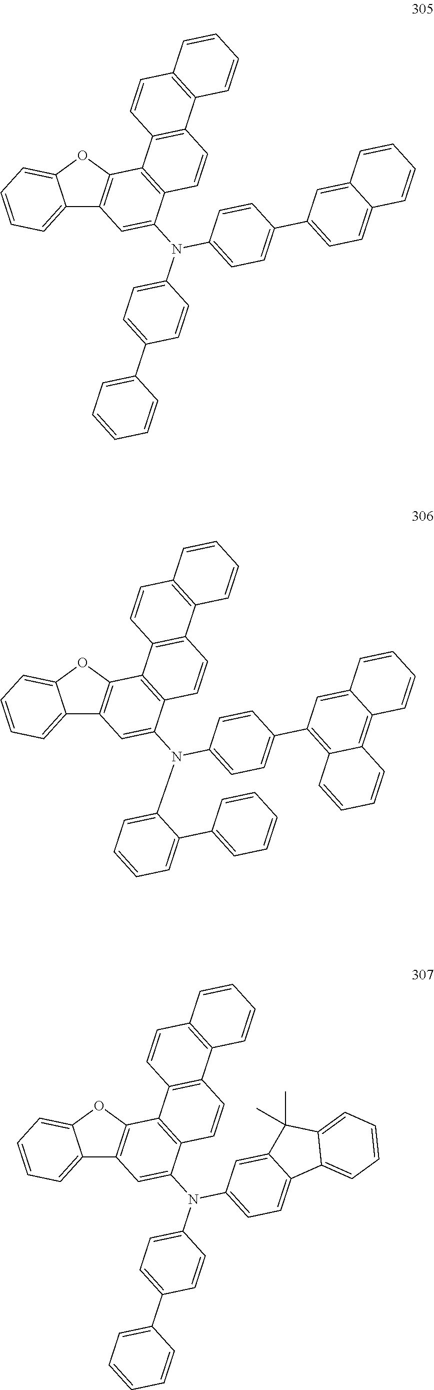 [SMILES: c1ccc(-c2ccc(N(c3ccc(-c4ccc5ccccc5c4)cc3)c3cc4c5ccccc5oc4c4c3ccc3c5ccccc5ccc34)cc2)cc1 ; c1ccc(-c2ccccc2N(c2ccc(-c3cc4ccccc4c4ccccc34)cc2)c2cc3c4ccccc4oc3c3c2ccc2c4ccccc4ccc23)cc1 ; CC1(C)c2ccccc2-c2ccc(N(c3ccc(-c4ccccc4)cc3)c3cc4c5ccccc5oc4c4c3ccc3c5ccccc5ccc34)cc21]

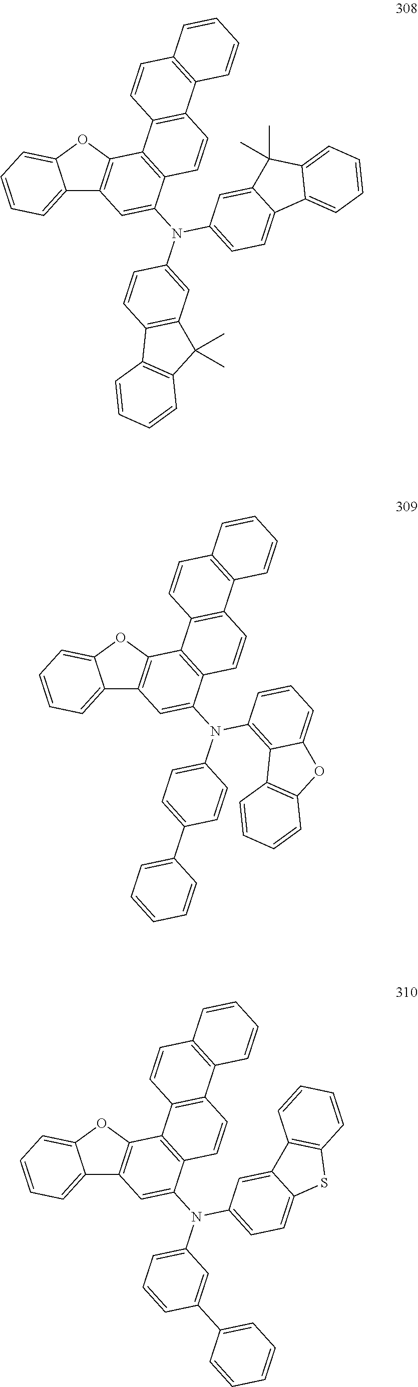 [SMILES: CC1(C)c2ccccc2-c2ccc(N(c3ccc4c(c3)C(C)(C)c3ccccc3-4)c3cc4c5ccccc5oc4c4c3ccc3c5ccccc5ccc34)cc21 ; c1ccc(-c2ccc(N(c3cc4c5ccccc5oc4c4c3ccc3c5ccccc5ccc34)c3cccc4oc5ccccc5c34)cc2)cc1 ; c1ccc(-c2cccc(N(c3ccc4sc5ccccc5c4c3)c3cc4c5ccccc5oc4c4c3ccc3c5ccccc5ccc34)c2)cc1]

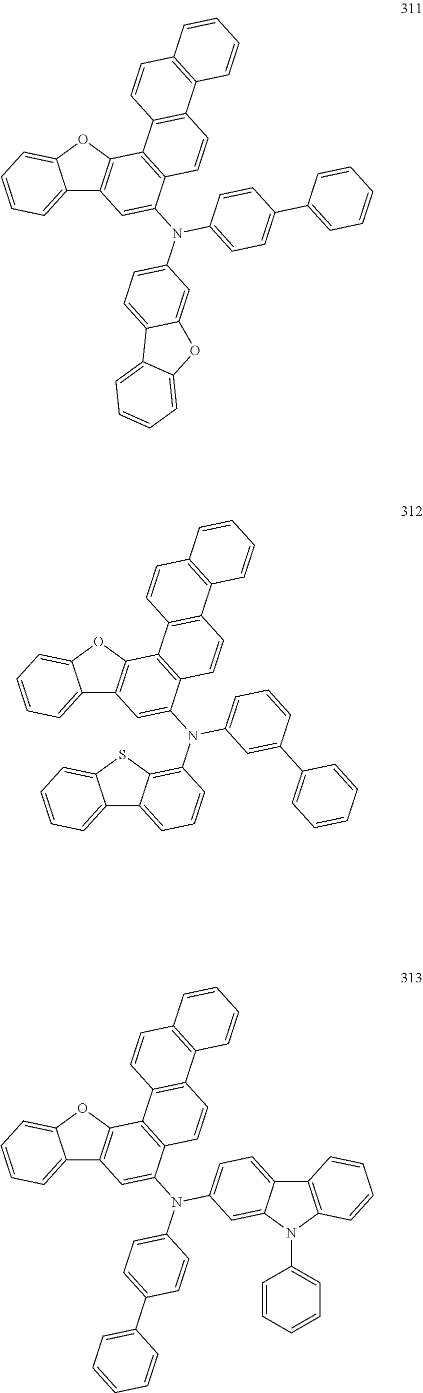 [SMILES: c1ccc(-c2ccc(N(c3ccc4c(c3)oc3ccccc34)c3cc4c5ccccc5oc4c4c3ccc3c5ccccc5ccc34)cc2)cc1 ; c1ccc(-c2cccc(N(c3cc4c5ccccc5oc4c4c3ccc3c5ccccc5ccc34)c3cccc4c3sc3ccccc34)c2)cc1 ; c1ccc(-c2ccc(N(c3ccc4c5ccccc5n(-c5ccccc5)c4c3)c3cc4c5ccccc5oc4c4c3ccc3c5ccccc5ccc34)cc2)cc1]

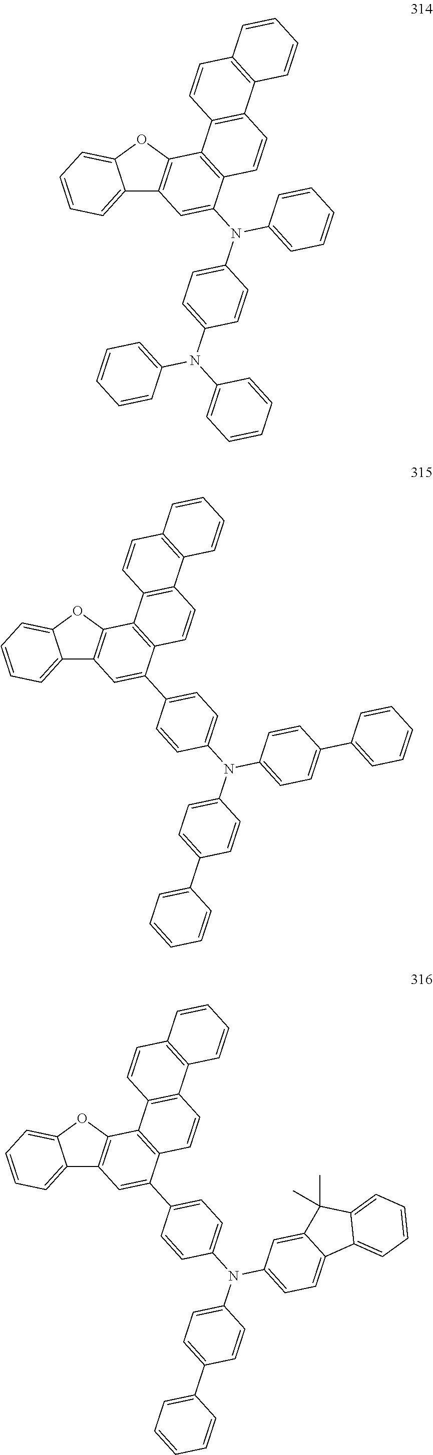 [SMILES: c1ccc(N(c2ccccc2)c2ccc(N(c3ccccc3)c3cc4c5ccccc5oc4c4c3ccc3c5ccccc5ccc34)cc2)cc1 ; c1ccc(-c2ccc(N(c3ccc(-c4ccccc4)cc3)c3ccc(-c4cc5c6ccccc6oc5c5c4ccc4c6ccccc6ccc45)cc3)cc2)cc1 ; CC1(C)c2ccccc2-c2ccc(N(c3ccc(-c4ccccc4)cc3)c3ccc(-c4cc5c6ccccc6oc5c5c4ccc4c6ccccc6ccc45)cc3)cc21]

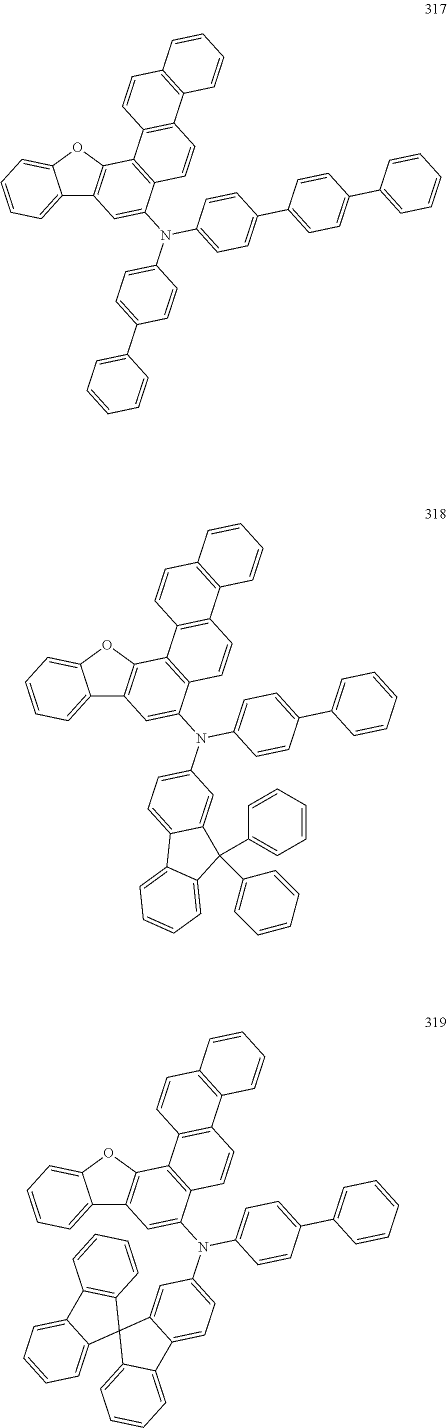 [SMILES: c1ccc(-c2ccc(-c3ccc(N(c4ccc(-c5ccccc5)cc4)c4cc5c6ccccc6oc5c5c4ccc4c6ccccc6ccc45)cc3)cc2)cc1 ; c1ccc(-c2ccc(N(c3ccc4c(c3)C(c3ccccc3)(c3ccccc3)c3ccccc3-4)c3cc4c5ccccc5oc4c4c3ccc3c5ccccc5ccc34)cc2)cc1 ; c1ccc(-c2ccc(N(c3ccc4c(c3)C3(c5ccccc5-c5ccccc53)c3ccccc3-4)c3cc4c5ccccc5oc4c4c3ccc3c5ccccc5ccc34)cc2)cc1]

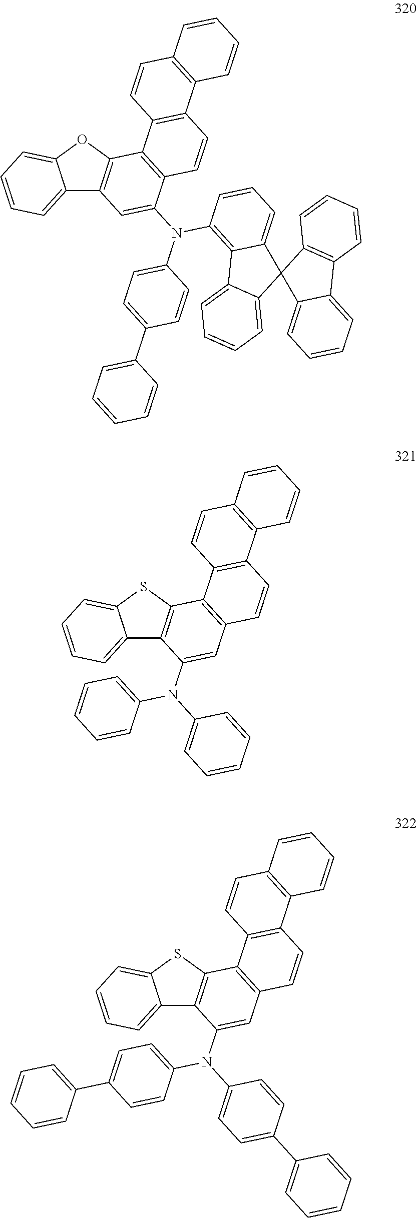 [SMILES: c1ccc(-c2ccc(N(c3cccc4c3-c3ccccc3C43c4ccccc4-c4ccccc43)c3cc4c5ccccc5oc4c4c3ccc3c5ccccc5ccc34)cc2)cc1 ; c1ccc(N(c2ccccc2)c2cc3ccc4c5ccccc5ccc4c3c3sc4ccccc4c23)cc1 ; c1ccc(-c2ccc(N(c3ccc(-c4ccccc4)cc3)c3cc4ccc5c6ccccc6ccc5c4c4sc5ccccc5c34)cc2)cc1]

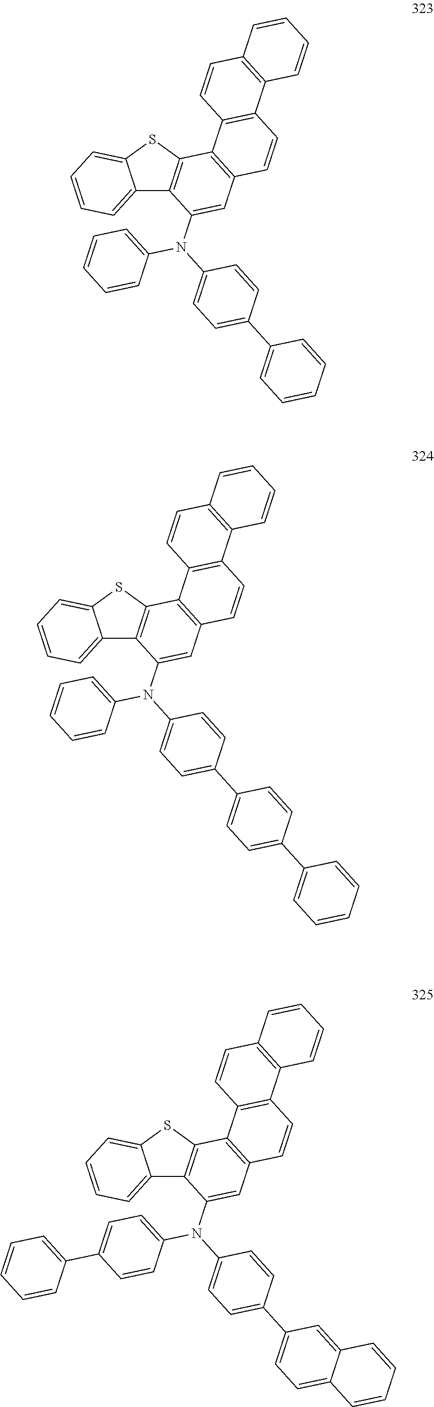 [SMILES: c1ccc(-c2ccc(N(c3ccccc3)c3cc4ccc5c6ccccc6ccc5c4c4sc5ccccc5c34)cc2)cc1 ; c1ccc(-c2ccc(-c3ccc(N(c4ccccc4)c4cc5ccc6c7ccccc7ccc6c5c5sc6ccccc6c45)cc3)cc2)cc1 ; c1ccc(-c2ccc(N(c3ccc(-c4ccc5ccccc5c4)cc3)c3cc4ccc5c6ccccc6ccc5c4c4sc5ccccc5c34)cc2)cc1]

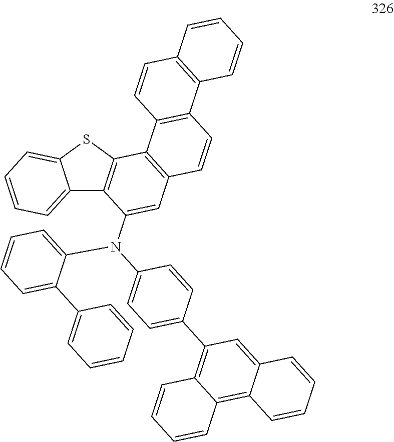 [SMILES: c1ccc(-c2ccccc2N(c2ccc(-c3cc4ccccc4c4ccccc34)cc2)c2cc3ccc4c5ccccc5ccc4c3c3sc4ccccc4c23)cc1]

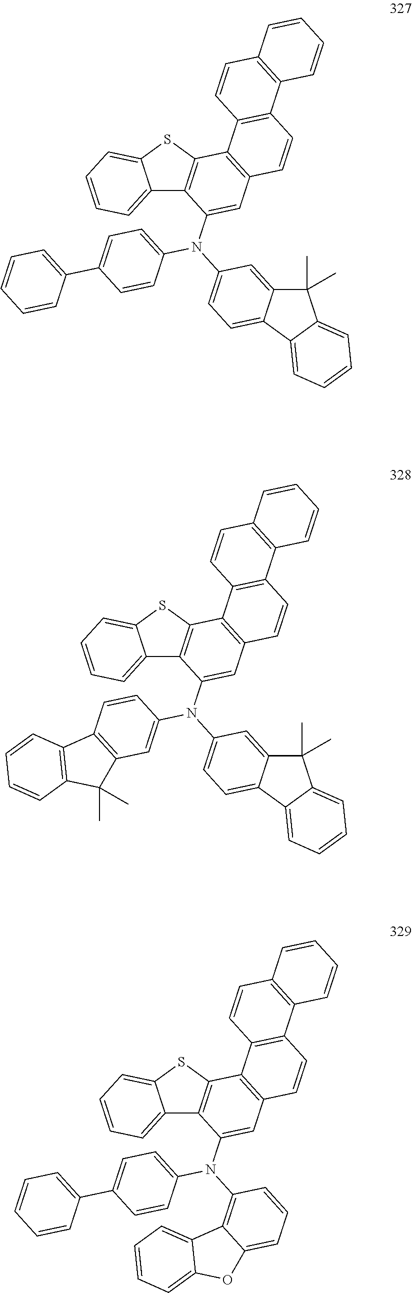 [SMILES: CC1(C)c2ccccc2-c2ccc(N(c3ccc(-c4ccccc4)cc3)c3cc4ccc5c6ccccc6ccc5c4c4sc5ccccc5c34)cc21 ; CC1(C)c2ccccc2-c2ccc(N(c3ccc4c(c3)C(C)(C)c3ccccc3-4)c3cc4ccc5c6ccccc6ccc5c4c4sc5ccccc5c34)cc21 ; c1ccc(-c2ccc(N(c3cccc4oc5ccccc5c34)c3cc4ccc5c6ccccc6ccc5c4c4sc5ccccc5c34)cc2)cc1]

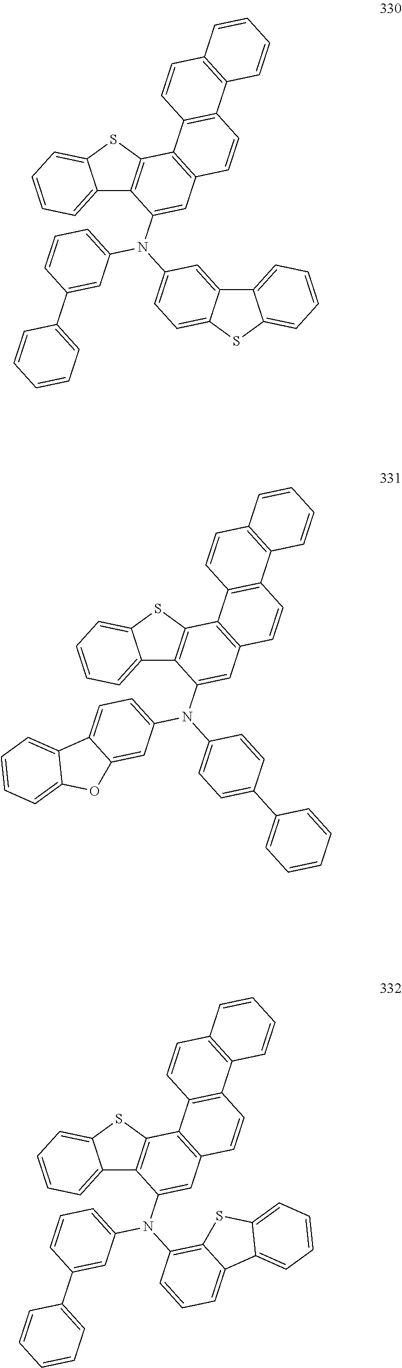 [SMILES: c1ccc(-c2cccc(N(c3ccc4sc5ccccc5c4c3)c3cc4ccc5c6ccccc6ccc5c4c4sc5ccccc5c34)c2)cc1 ; c1ccc(-c2ccc(N(c3ccc4c(c3)oc3ccccc34)c3cc4ccc5c6ccccc6ccc5c4c4sc5ccccc5c34)cc2)cc1 ; c1ccc(-c2cccc(N(c3cccc4c3sc3ccccc34)c3cc4ccc5c6ccccc6ccc5c4c4sc5ccccc5c34)c2)cc1]

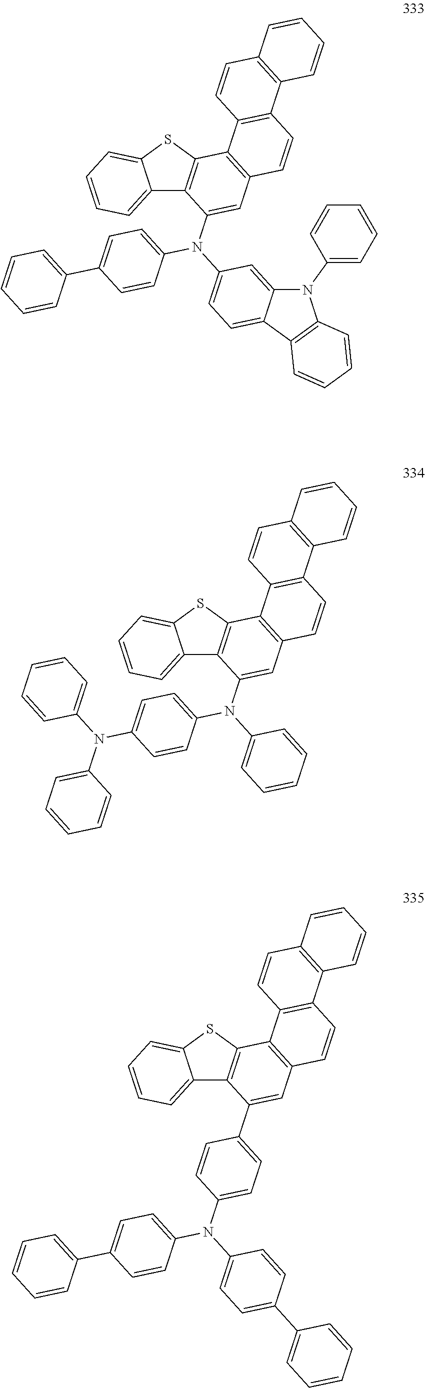 [SMILES: c1ccc(-c2ccc(N(c3ccc4c5ccccc5n(-c5ccccc5)c4c3)c3cc4ccc5c6ccccc6ccc5c4c4sc5ccccc5c34)cc2)cc1 ; c1ccc(N(c2ccccc2)c2ccc(N(c3ccccc3)c3cc4ccc5c6ccccc6ccc5c4c4sc5ccccc5c34)cc2)cc1 ; c1ccc(-c2ccc(N(c3ccc(-c4ccccc4)cc3)c3ccc(-c4cc5ccc6c7ccccc7ccc6c5c5sc6ccccc6c45)cc3)cc2)cc1]

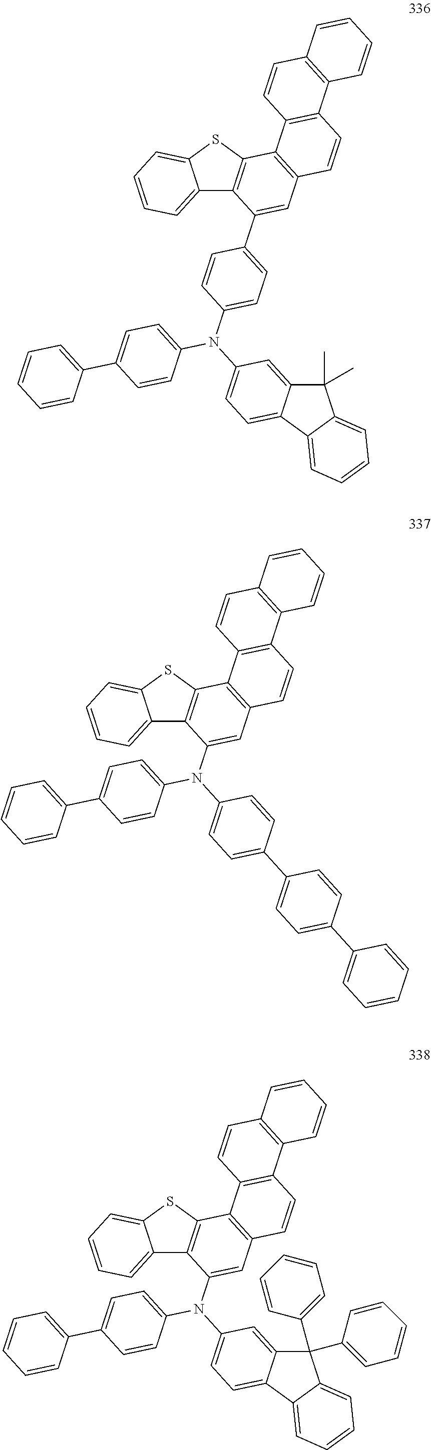 [SMILES: CC1(C)c2ccccc2-c2ccc(N(c3ccc(-c4ccccc4)cc3)c3ccc(-c4cc5ccc6c7ccccc7ccc6c5c5sc6ccccc6c45)cc3)cc21 ; c1ccc(-c2ccc(-c3ccc(N(c4ccc(-c5ccccc5)cc4)c4cc5ccc6c7ccccc7ccc6c5c5sc6ccccc6c45)cc3)cc2)cc1 ; c1ccc(-c2ccc(N(c3ccc4c(c3)C(c3ccccc3)(c3ccccc3)c3ccccc3-4)c3cc4ccc5c6ccccc6ccc5c4c4sc5ccccc5c34)cc2)cc1]

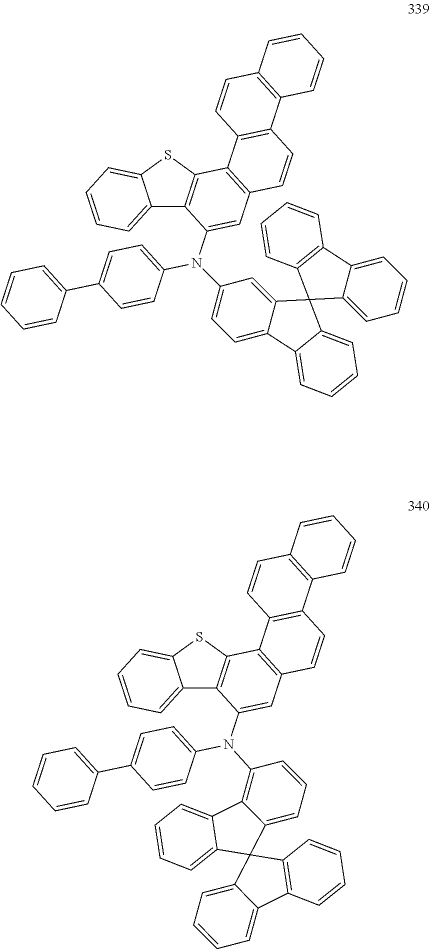 [SMILES: c1ccc(-c2ccc(N(c3ccc4c(c3)C3(c5ccccc5-c5ccccc53)c3ccccc3-4)c3cc4ccc5c6ccccc6ccc5c4c4sc5ccccc5c34)cc2)cc1 ; c1ccc(-c2ccc(N(c3cccc4c3-c3ccccc3C43c4ccccc4-c4ccccc43)c3cc4ccc5c6ccccc6ccc5c4c4sc5ccccc5c34)cc2)cc1]

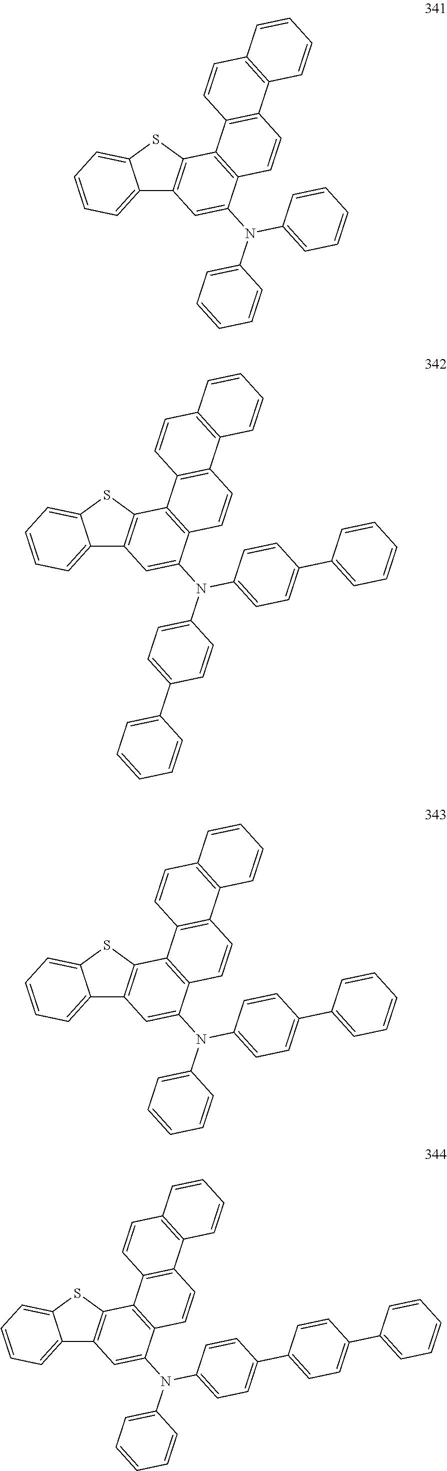 [SMILES: c1ccc(N(c2ccccc2)c2cc3c4ccccc4sc3c3c2ccc2c4ccccc4ccc23)cc1 ; c1ccc(-c2ccc(N(c3ccc(-c4ccccc4)cc3)c3cc4c5ccccc5sc4c4c3ccc3c5ccccc5ccc34)cc2)cc1 ; c1ccc(-c2ccc(N(c3ccccc3)c3cc4c5ccccc5sc4c4c3ccc3c5ccccc5ccc34)cc2)cc1 ; c1ccc(-c2ccc(-c3ccc(N(c4ccccc4)c4cc5c6ccccc6sc5c5c4ccc4c6ccccc6ccc45)cc3)cc2)cc1]

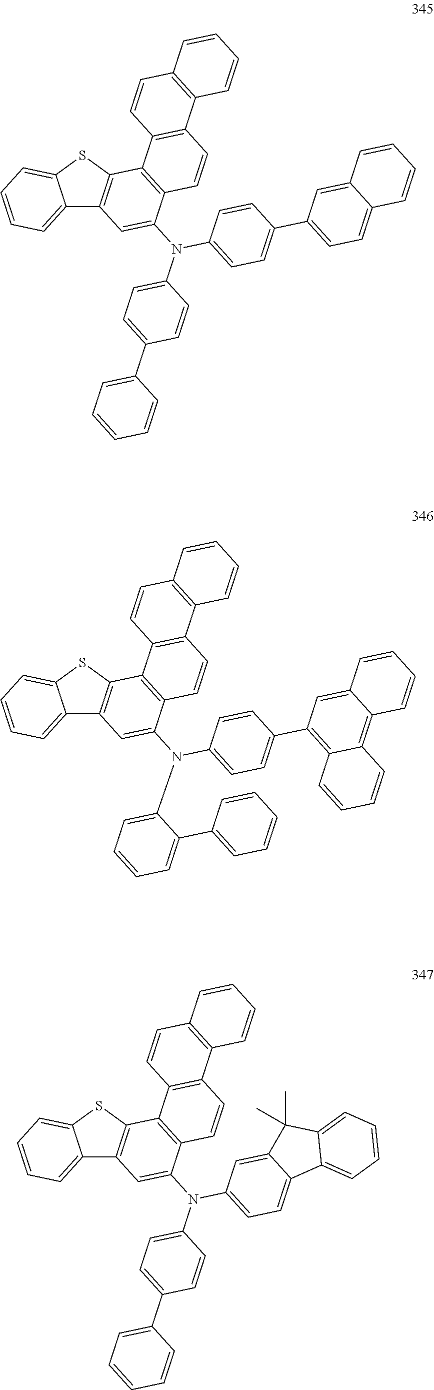 [SMILES: c1ccc(-c2ccc(N(c3ccc(-c4ccc5ccccc5c4)cc3)c3cc4c5ccccc5sc4c4c3ccc3c5ccccc5ccc34)cc2)cc1 ; c1ccc(-c2ccccc2N(c2ccc(-c3cc4ccccc4c4ccccc34)cc2)c2cc3c4ccccc4sc3c3c2ccc2c4ccccc4ccc23)cc1 ; CC1(C)c2ccccc2-c2ccc(N(c3ccc(-c4ccccc4)cc3)c3cc4c5ccccc5sc4c4c3ccc3c5ccccc5ccc34)cc21]

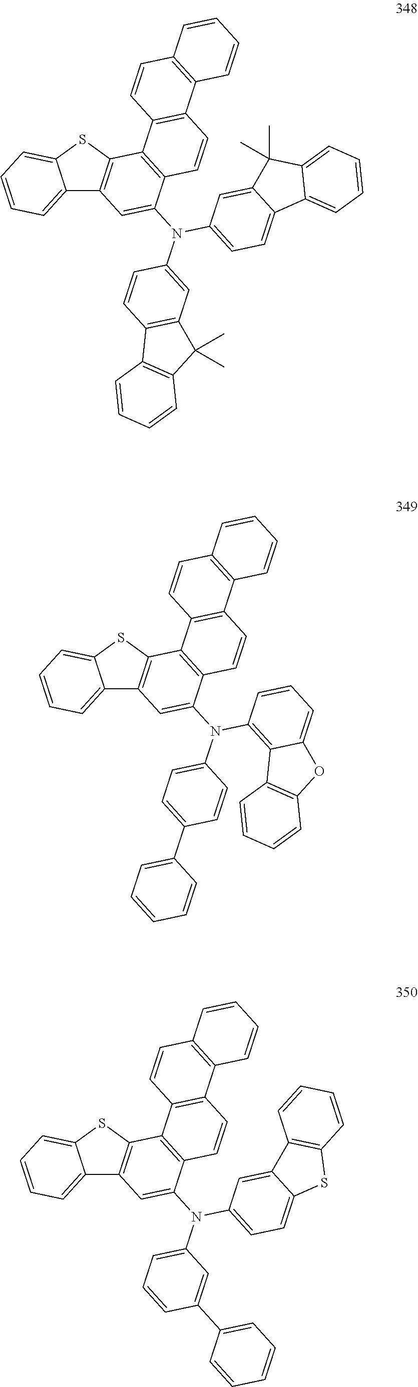 [SMILES: CC1(C)c2ccccc2-c2ccc(N(c3ccc4c(c3)C(C)(C)c3ccccc3-4)c3cc4c5ccccc5sc4c4c3ccc3c5ccccc5ccc34)cc21 ; c1ccc(-c2ccc(N(c3cc4c5ccccc5sc4c4c3ccc3c5ccccc5ccc34)c3cccc4oc5ccccc5c34)cc2)cc1 ; c1ccc(-c2cccc(N(c3ccc4sc5ccccc5c4c3)c3cc4c5ccccc5sc4c4c3ccc3c5ccccc5ccc34)c2)cc1]

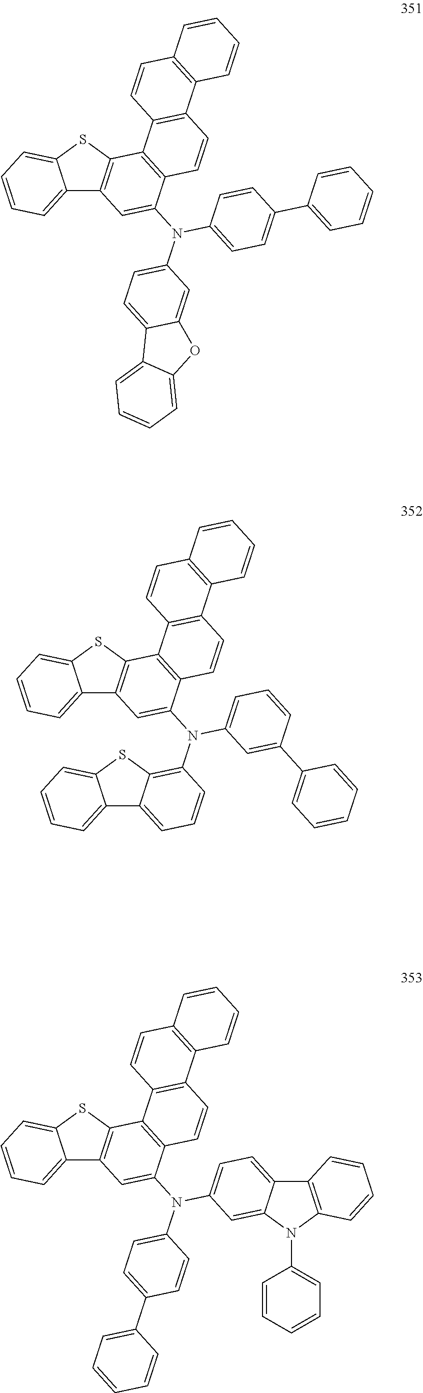 [SMILES: c1ccc(-c2ccc(N(c3ccc4c(c3)oc3ccccc34)c3cc4c5ccccc5sc4c4c3ccc3c5ccccc5ccc34)cc2)cc1 ; c1ccc(-c2cccc(N(c3cc4c5ccccc5sc4c4c3ccc3c5ccccc5ccc34)c3cccc4c3sc3ccccc34)c2)cc1 ; c1ccc(-c2ccc(N(c3ccc4c5ccccc5n(-c5ccccc5)c4c3)c3cc4c5ccccc5sc4c4c3ccc3c5ccccc5ccc34)cc2)cc1]

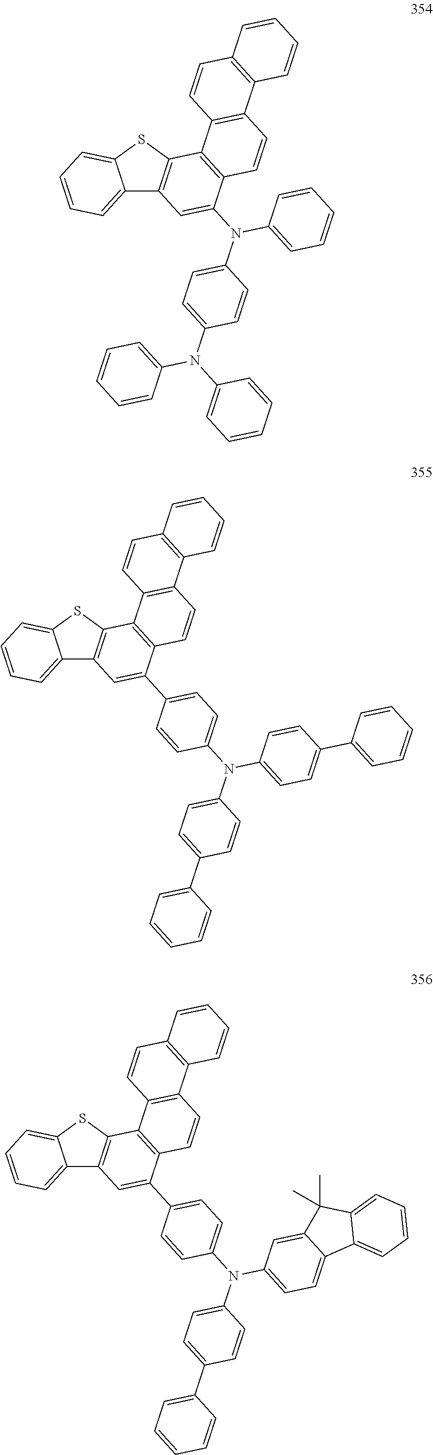 [SMILES: c1ccc(N(c2ccccc2)c2ccc(N(c3ccccc3)c3cc4c5ccccc5sc4c4c3ccc3c5ccccc5ccc34)cc2)cc1 ; c1ccc(-c2ccc(N(c3ccc(-c4ccccc4)cc3)c3ccc(-c4cc5c6ccccc6sc5c5c4ccc4c6ccccc6ccc45)cc3)cc2)cc1 ; CC1(C)c2ccccc2-c2ccc(N(c3ccc(-c4ccccc4)cc3)c3ccc(-c4cc5c6ccccc6sc5c5c4ccc4c6ccccc6ccc45)cc3)cc21]

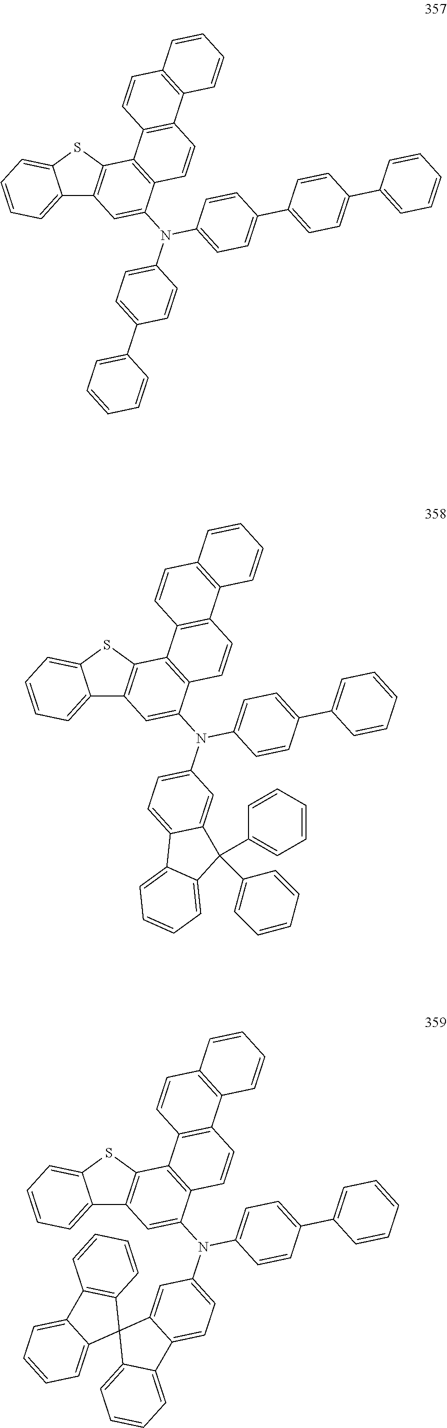 [SMILES: c1ccc(-c2ccc(-c3ccc(N(c4ccc(-c5ccccc5)cc4)c4cc5c6ccccc6sc5c5c4ccc4c6ccccc6ccc45)cc3)cc2)cc1 ; c1ccc(-c2ccc(N(c3ccc4c(c3)C(c3ccccc3)(c3ccccc3)c3ccccc3-4)c3cc4c5ccccc5sc4c4c3ccc3c5ccccc5ccc34)cc2)cc1 ; c1ccc(-c2ccc(N(c3ccc4c(c3)C3(c5ccccc5-c5ccccc53)c3ccccc3-4)c3cc4c5ccccc5sc4c4c3ccc3c5ccccc5ccc34)cc2)cc1]

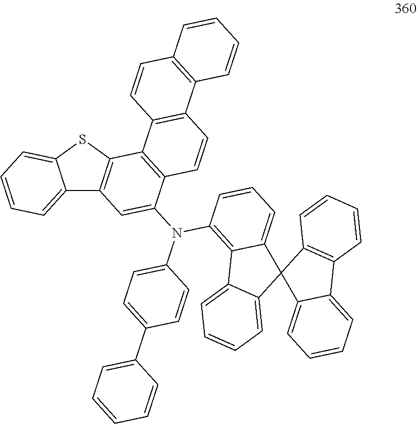 [SMILES: c1ccc(-c2ccc(N(c3cccc4c3-c3ccccc3C43c4ccccc4-c4ccccc43)c3cc4c5ccccc5sc4c4c3ccc3c5ccccc5ccc34)cc2)cc1]

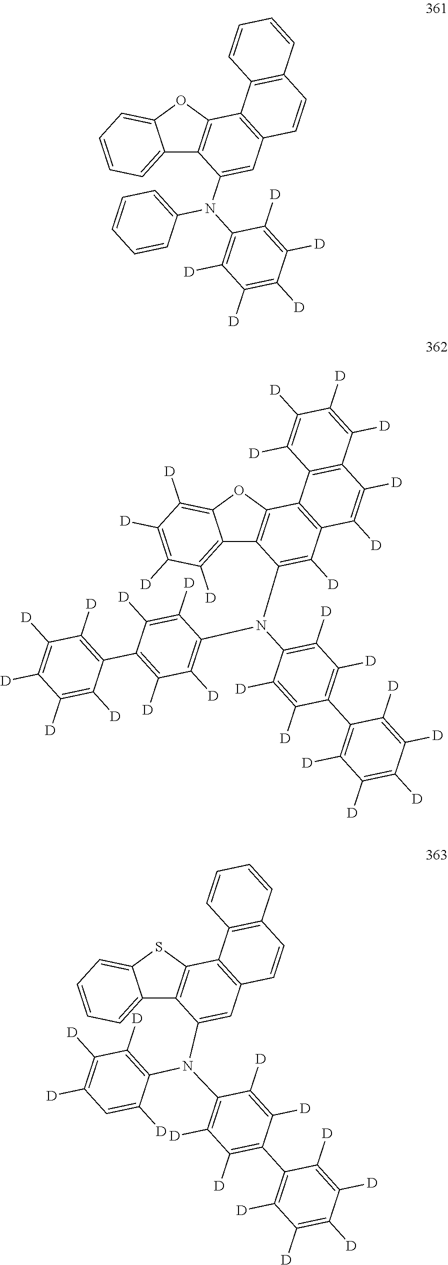 [SMILES: [2H]c1c([2H])c([2H])c(N(c2ccccc2)c2cc3ccc4ccccc4c3c3oc4ccccc4c23)c([2H])c1[2H] ; [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c4c([2H])c([2H])c5c([2H])c([2H])c([2H])c([2H])c5c4c4oc5c([2H])c([2H])c([2H])c([2H])c5c34)c([2H])c2[2H])c([2H])c1[2H] ; [2H]c1cc([2H])c(N(c2c([2H])c([2H])c(-c3c([2H])c([2H])c([2H])c([2H])c3[2H])c([2H])c2[2H])c2cc3ccc4ccccc4c3c3sc4ccccc4c23)c([2H])c1[2H]]

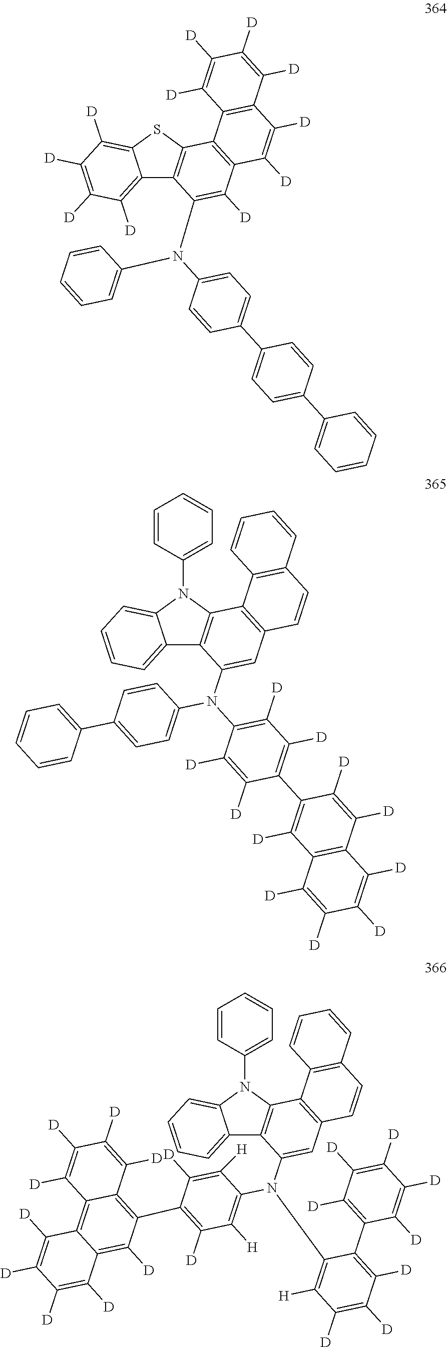 [SMILES: [2H]c1c([2H])c([2H])c2c(sc3c2c(N(c2ccccc2)c2ccc(-c4ccc(-c5ccccc5)cc4)cc2)c([2H])c2c([2H])c([2H])c4c([2H])c([2H])c([2H])c([2H])c4c23)c1[2H] ; [2H]c1c([2H])c(N(c2ccc(-c3ccccc3)cc2)c2cc3ccc4ccccc4c3c3c2c2ccccc2n3-c2ccccc2)c([2H])c([2H])c1-c1c([2H])c([2H])c2c([2H])c([2H])c([2H])c([2H])c2c1[2H] ; [2H]c1cc(N(c2cc([2H])c(-c3c([2H])c4c([2H])c([2H])c([2H])c([2H])c4c4c([2H])c([2H])c([2H])c([2H])c34)c([2H])c2)c2cc3ccc4ccccc4c3c3c2c2ccccc2n3-c2ccccc2)c(-c2c([2H])c([2H])c([2H])c([2H])c2[2H])c([2H])c1[2H]]

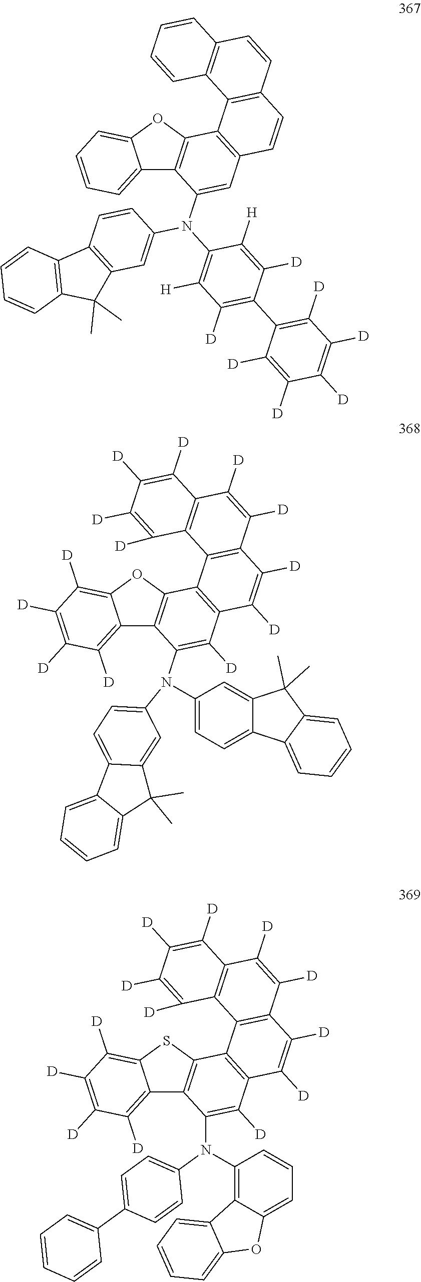 [SMILES: [2H]c1cc(N(c2ccc3c(c2)C(C)(C)c2ccccc2-3)c2cc3ccc4ccc5ccccc5c4c3c3oc4ccccc4c23)cc([2H])c1-c1c([2H])c([2H])c([2H])c([2H])c1[2H] ; [2H]c1c([2H])c([2H])c2c(oc3c2c(N(c2ccc4c(c2)C(C)(C)c2ccccc2-4)c2ccc4c(c2)C(C)(C)c2ccccc2-4)c([2H])c2c([2H])c([2H])c4c([2H])c([2H])c5c([2H])c([2H])c([2H])c([2H])c5c4c23)c1[2H] ; [2H]c1c([2H])c([2H])c2c(sc3c2c(N(c2ccc(-c4ccccc4)cc2)c2cccc4oc5ccccc5c24)c([2H])c2c([2H])c([2H])c4c([2H])c([2H])c5c([2H])c([2H])c([2H])c([2H])c5c4c23)c1[2H]]

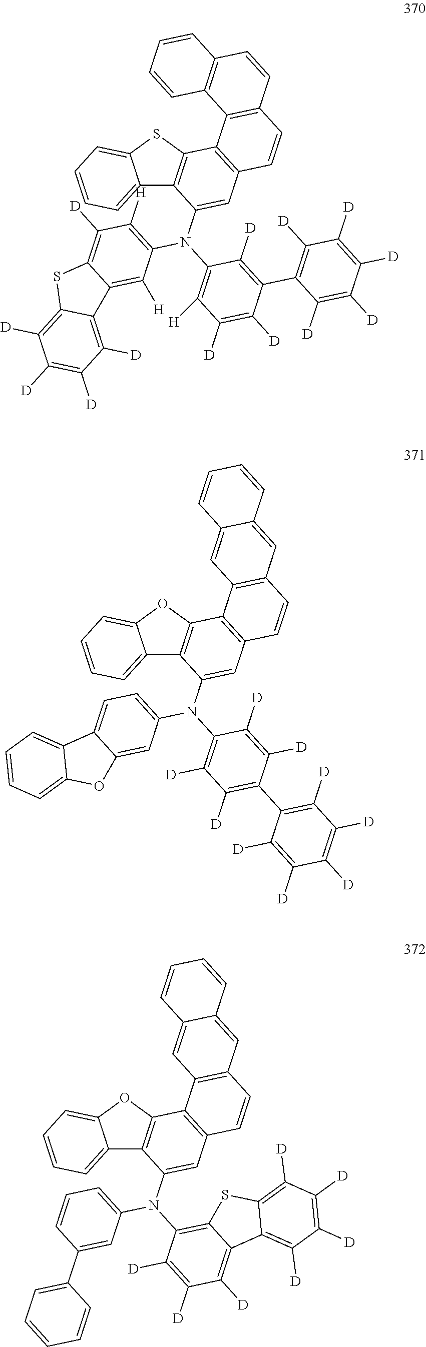 [SMILES: [2H]c1cc(N(c2cc([2H])c3sc4c([2H])c([2H])c([2H])c([2H])c4c3c2)c2cc3ccc4ccc5ccccc5c4c3c3sc4ccccc4c23)c([2H])c(-c2c([2H])c([2H])c([2H])c([2H])c2[2H])c1[2H] ; [2H]c1c([2H])c([2H])c(-c2c([2H])c([2H])c(N(c3ccc4c(c3)oc3ccccc34)c3cc4ccc5cc6ccccc6cc5c4c4oc5ccccc5c34)c([2H])c2[2H])c([2H])c1[2H] ; [2H]c1c([2H])c([2H])c2c(sc3c(N(c4cccc(-c5ccccc5)c4)c4cc5ccc6cc7ccccc7cc6c5c5oc6ccccc6c45)c([2H])c([2H])c([2H])c32)c1[2H]]

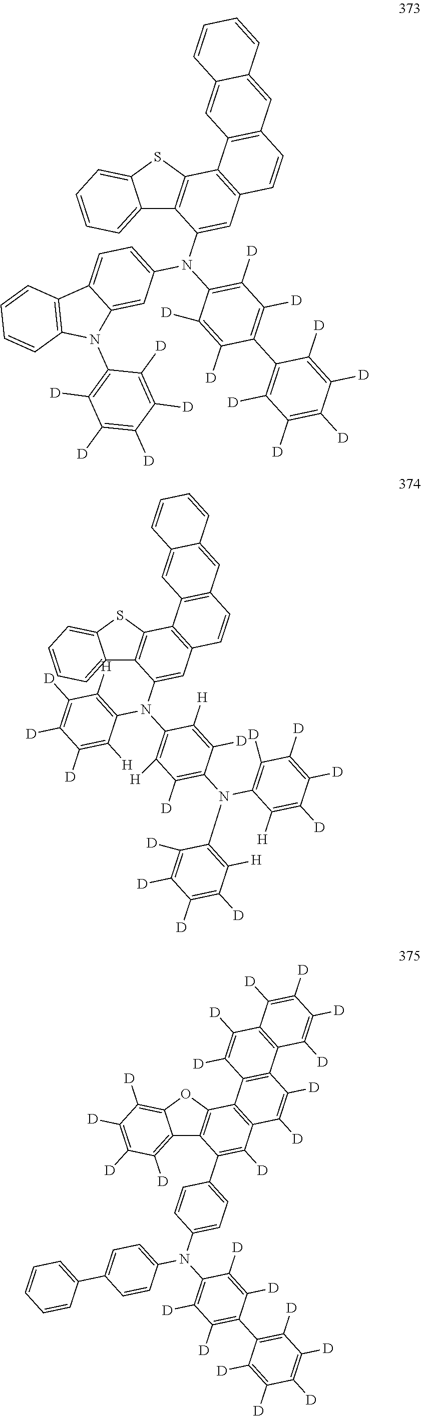

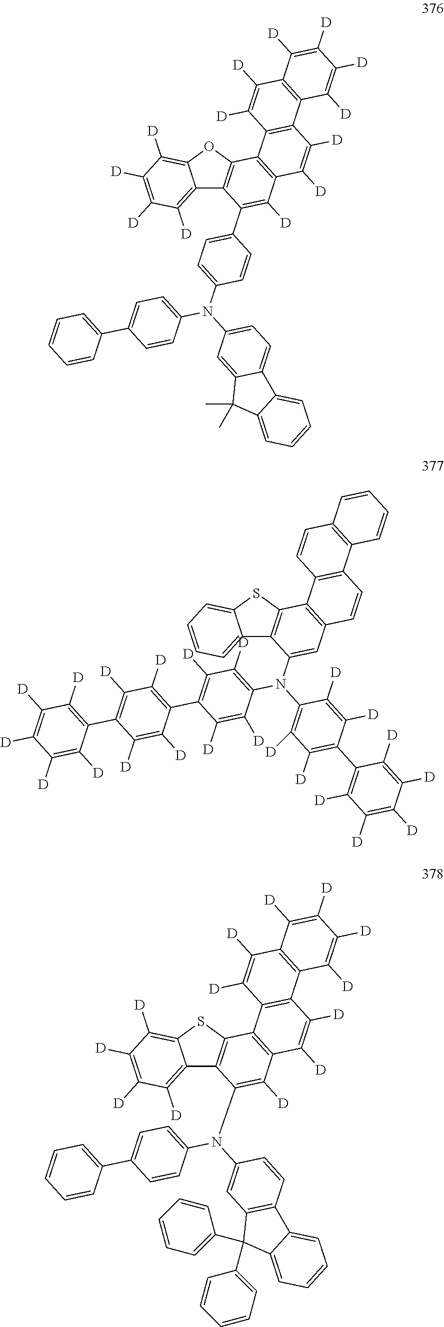

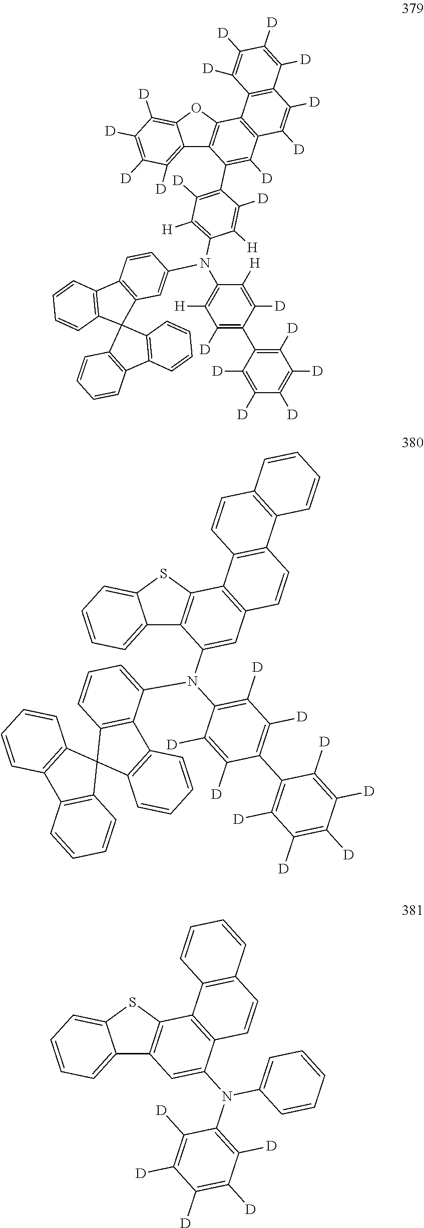

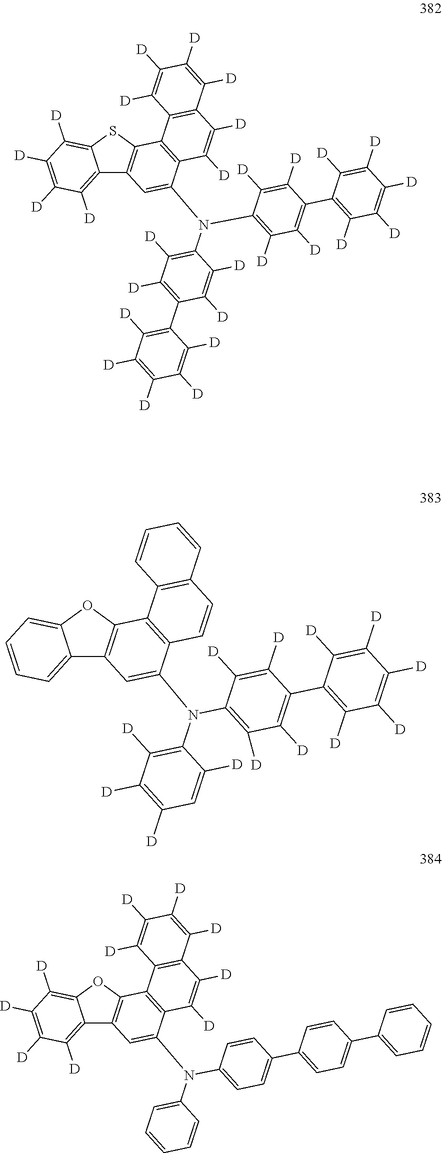

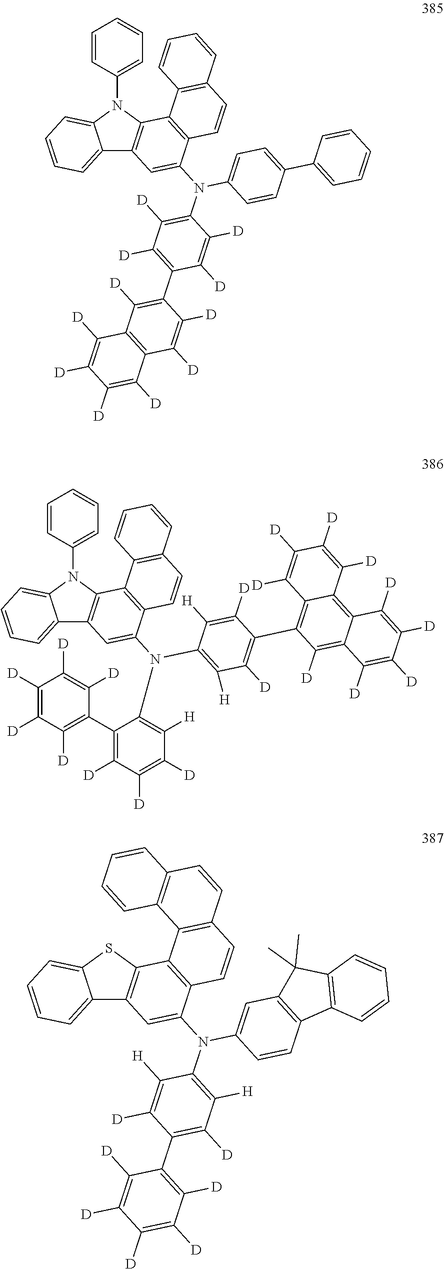 [SMILES: [2H]c1c([2H])c(N(c2ccc(-c3ccccc3)cc2)c2cc3c4ccccc4n(-c4ccccc4)c3c3c2ccc2ccccc23)c([2H])c([2H])c1-c1c([2H])c([2H])c2c([2H])c([2H])c([2H])c([2H])c2c1[2H] ; [2H]c1cc(N(c2cc([2H])c(-c3c([2H])c4c([2H])c([2H])c([2H])c([2H])c4c4c([2H])c([2H])c([2H])c([2H])c34)c([2H])c2)c2cc3c4ccccc4n(-c4ccccc4)c3c3c2ccc2ccccc23)c(-c2c([2H])c([2H])c([2H])c([2H])c2[2H])c([2H])c1[2H] ; [2H]c1cc(N(c2ccc3c(c2)C(C)(C)c2ccccc2-3)c2cc3c4ccccc4sc3c3c2ccc2ccc4ccccc4c23)cc([2H])c1-c1c([2H])c([2H])c([2H])c([2H])c1[2H]]

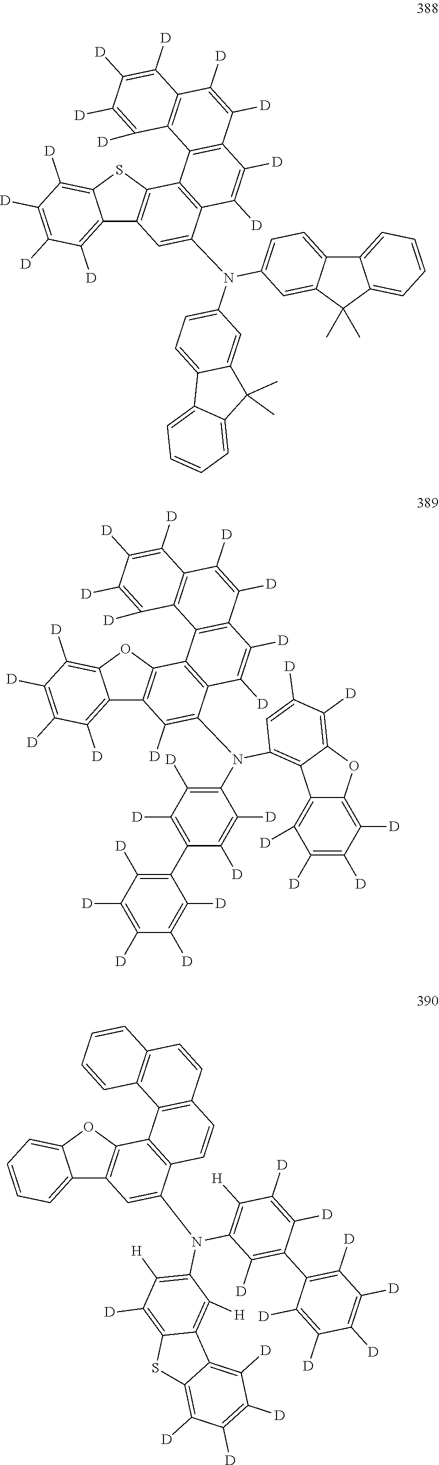

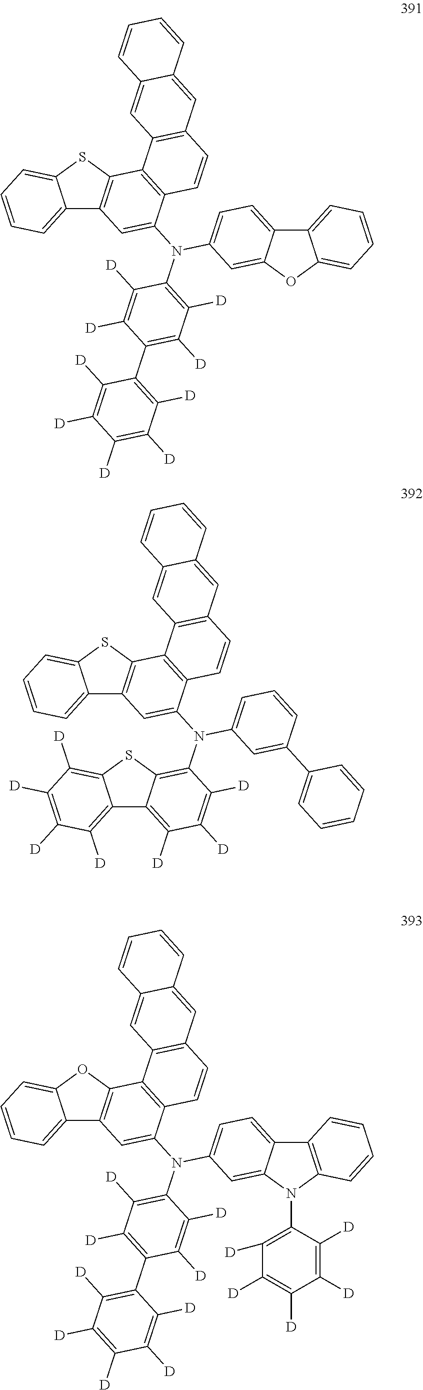

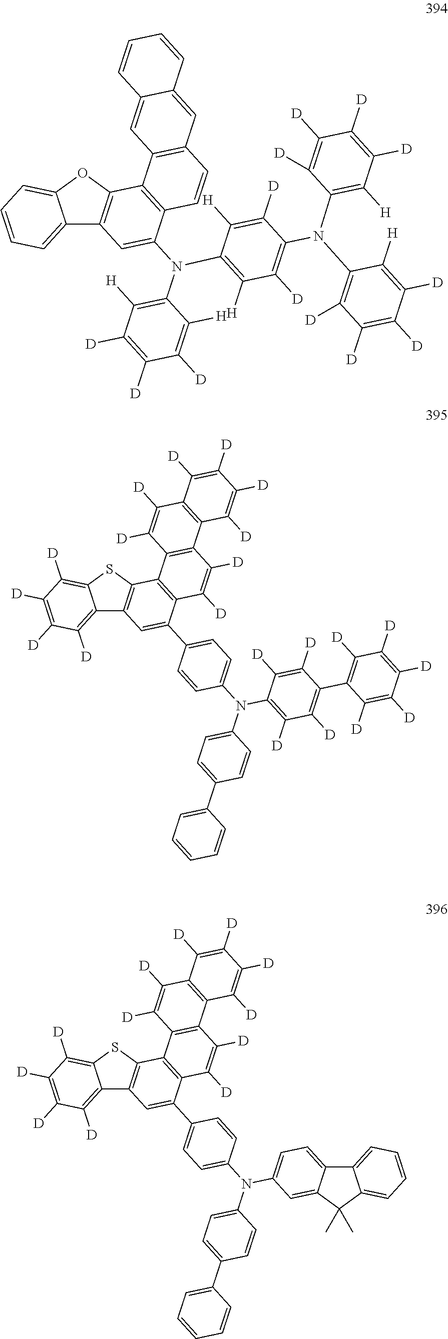

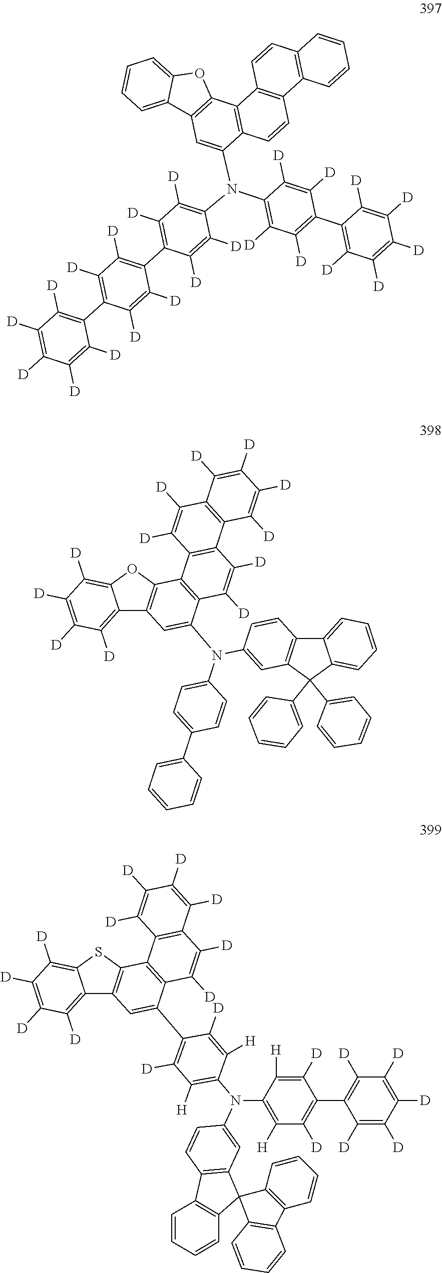

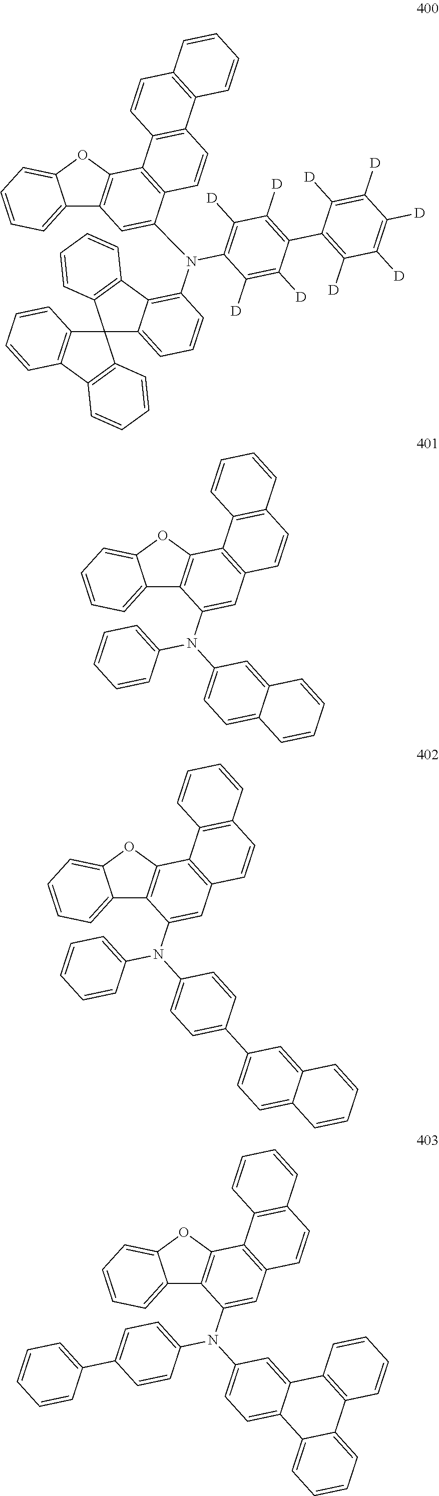

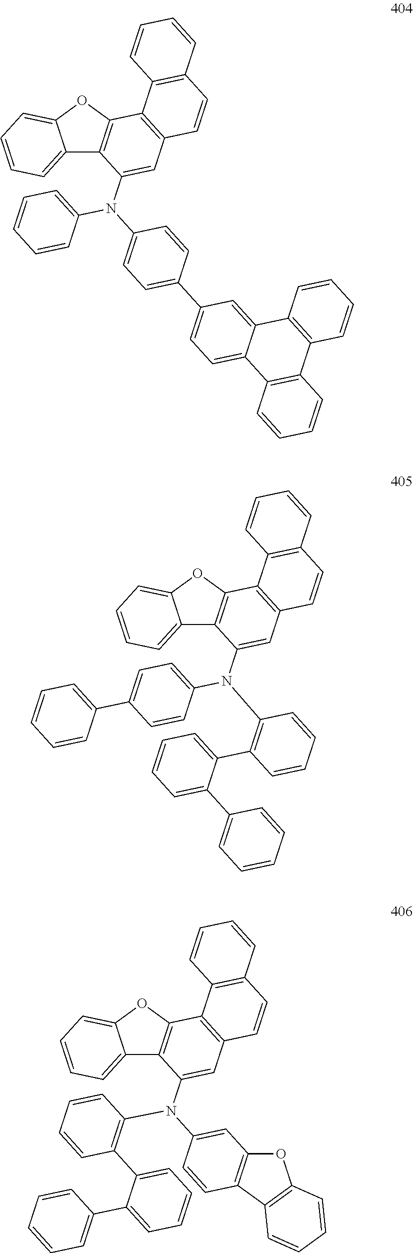 [SMILES: c1ccc(N(c2ccc(-c3ccc4c5ccccc5c5ccccc5c4c3)cc2)c2cc3ccc4ccccc4c3c3oc4ccccc4c23)cc1 ; c1ccc(-c2ccc(N(c3ccccc3-c3ccccc3-c3ccccc3)c3cc4ccc5ccccc5c4c4oc5ccccc5c34)cc2)cc1 ; c1ccc(-c2ccccc2-c2ccccc2N(c2ccc3c(c2)oc2ccccc23)c2cc3ccc4ccccc4c3c3oc4ccccc4c23)cc1]

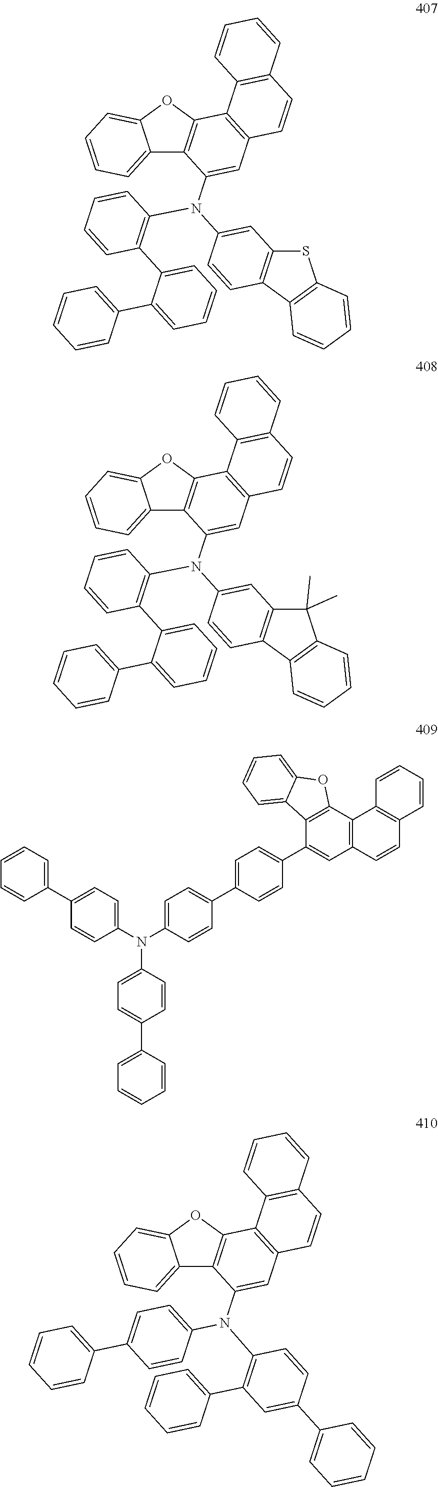 [SMILES: c1ccc(-c2ccccc2-c2ccccc2N(c2ccc3c(c2)sc2ccccc23)c2cc3ccc4ccccc4c3c3oc4ccccc4c23)cc1 ; CC1(C)c2ccccc2-c2ccc(N(c3ccccc3-c3ccccc3-c3ccccc3)c3cc4ccc5ccccc5c4c4oc5ccccc5c34)cc21 ; c1ccc(-c2ccc(N(c3ccc(-c4ccccc4)cc3)c3ccc(-c4ccc(-c5cc6ccc7ccccc7c6c6oc7ccccc7c56)cc4)cc3)cc2)cc1 ; c1ccc(-c2ccc(N(c3ccc(-c4ccccc4)cc3-c3ccccc3)c3cc4ccc5ccccc5c4c4oc5ccccc5c34)cc2)cc1]

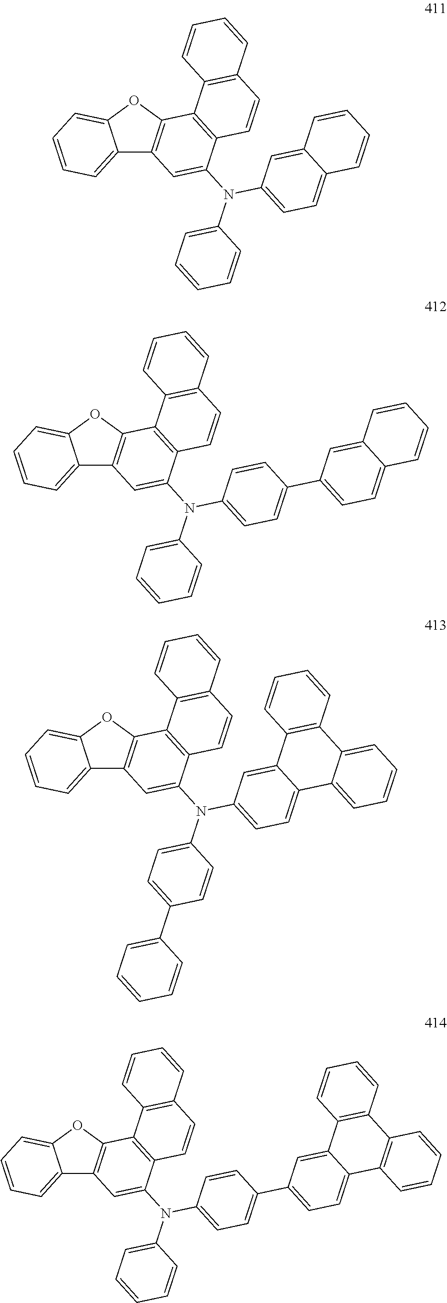

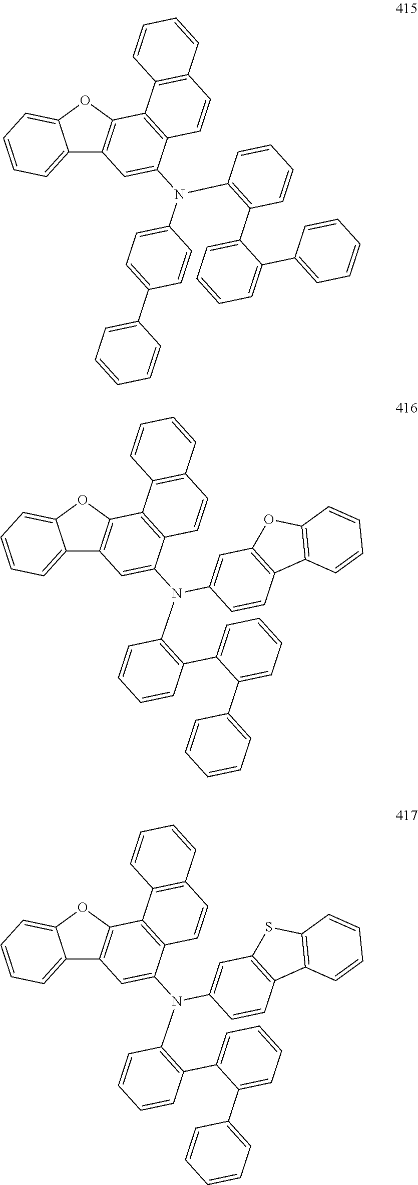

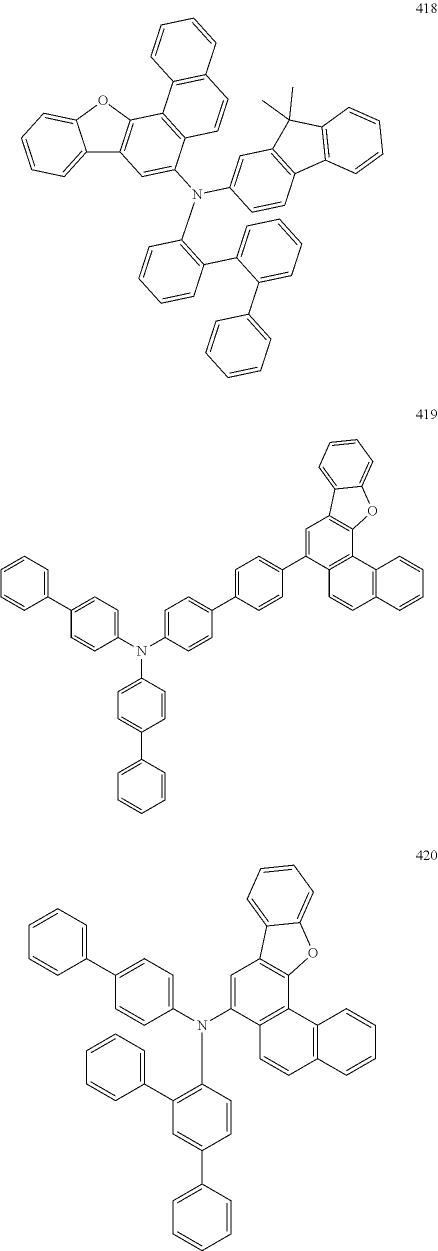 [SMILES: CC1(C)c2ccccc2-c2ccc(N(c3ccccc3-c3ccccc3-c3ccccc3)c3cc4c5ccccc5oc4c4c3ccc3ccccc34)cc21 ; c1ccc(-c2ccc(N(c3ccc(-c4ccccc4)cc3)c3ccc(-c4ccc(-c5cc6c7ccccc7oc6c6c5ccc5ccccc56)cc4)cc3)cc2)cc1 ; c1ccc(-c2ccc(N(c3ccc(-c4ccccc4)cc3-c3ccccc3)c3cc4c5ccccc5oc4c4c3ccc3ccccc34)cc2)cc1]

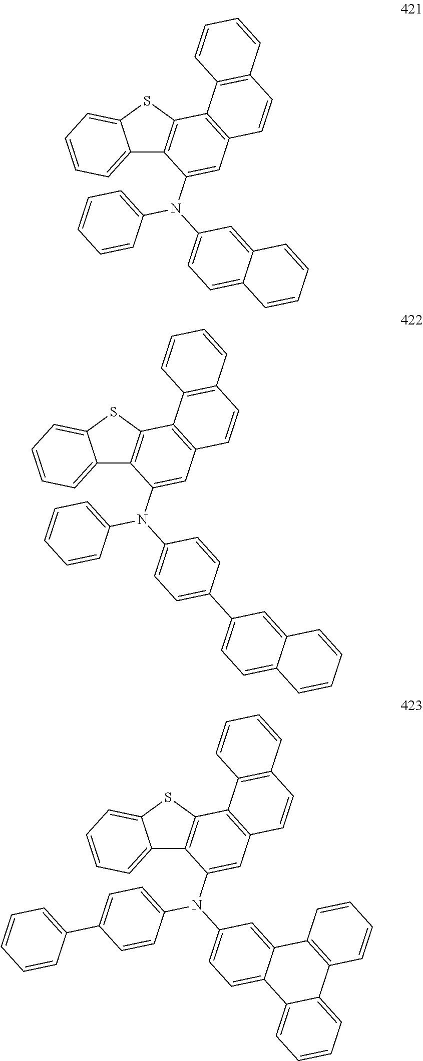

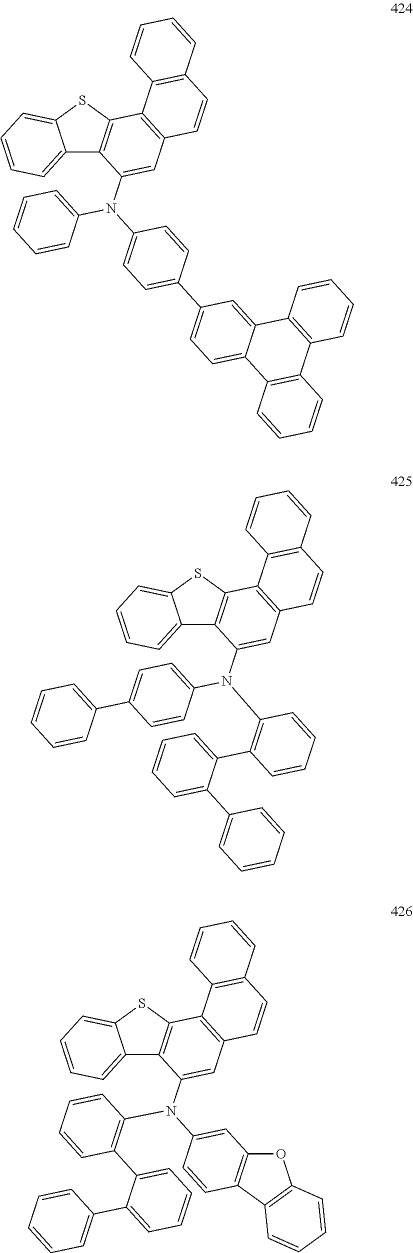 [SMILES: c1ccc(N(c2ccc(-c3ccc4c5ccccc5c5ccccc5c4c3)cc2)c2cc3ccc4ccccc4c3c3sc4ccccc4c23)cc1 ; c1ccc(-c2ccc(N(c3ccccc3-c3ccccc3-c3ccccc3)c3cc4ccc5ccccc5c4c4sc5ccccc5c34)cc2)cc1 ; c1ccc(-c2ccccc2-c2ccccc2N(c2ccc3c(c2)oc2ccccc23)c2cc3ccc4ccccc4c3c3sc4ccccc4c23)cc1]

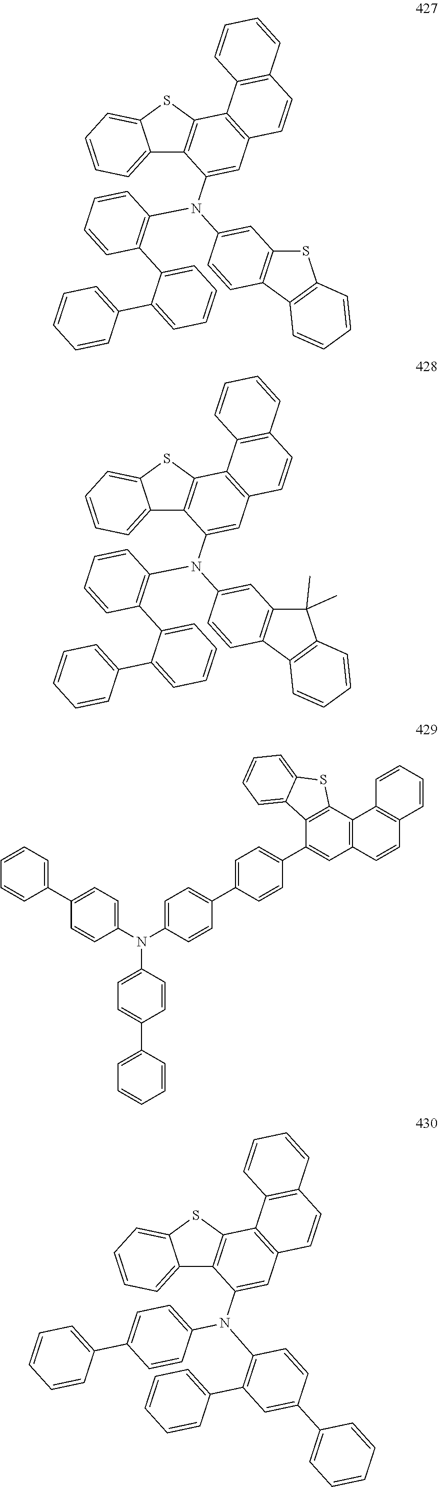 [SMILES: c1ccc(-c2ccccc2-c2ccccc2N(c2ccc3c(c2)sc2ccccc23)c2cc3ccc4ccccc4c3c3sc4ccccc4c23)cc1 ; CC1(C)c2ccccc2-c2ccc(N(c3ccccc3-c3ccccc3-c3ccccc3)c3cc4ccc5ccccc5c4c4sc5ccccc5c34)cc21 ; c1ccc(-c2ccc(N(c3ccc(-c4ccccc4)cc3)c3ccc(-c4ccc(-c5cc6ccc7ccccc7c6c6sc7ccccc7c56)cc4)cc3)cc2)cc1 ; c1ccc(-c2ccc(N(c3ccc(-c4ccccc4)cc3-c3ccccc3)c3cc4ccc5ccccc5c4c4sc5ccccc5c34)cc2)cc1]

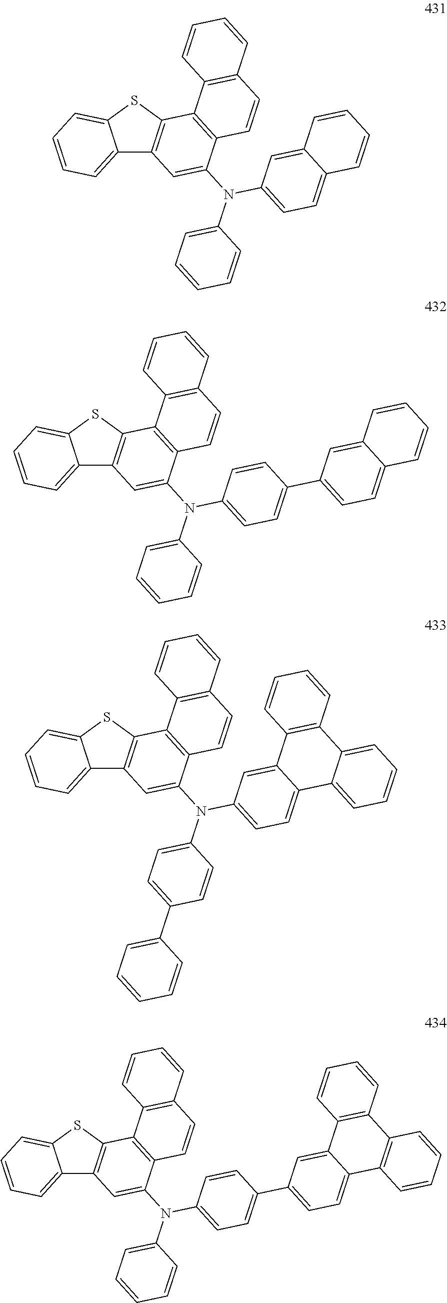

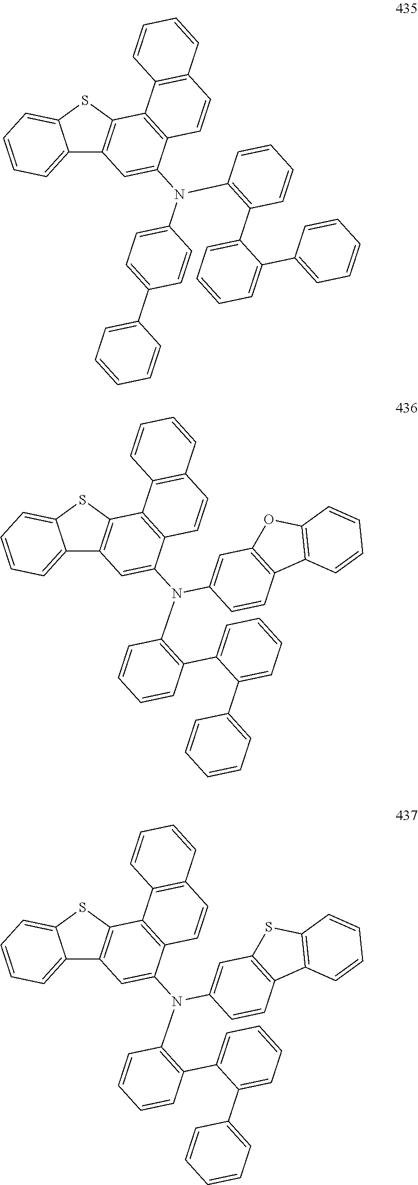

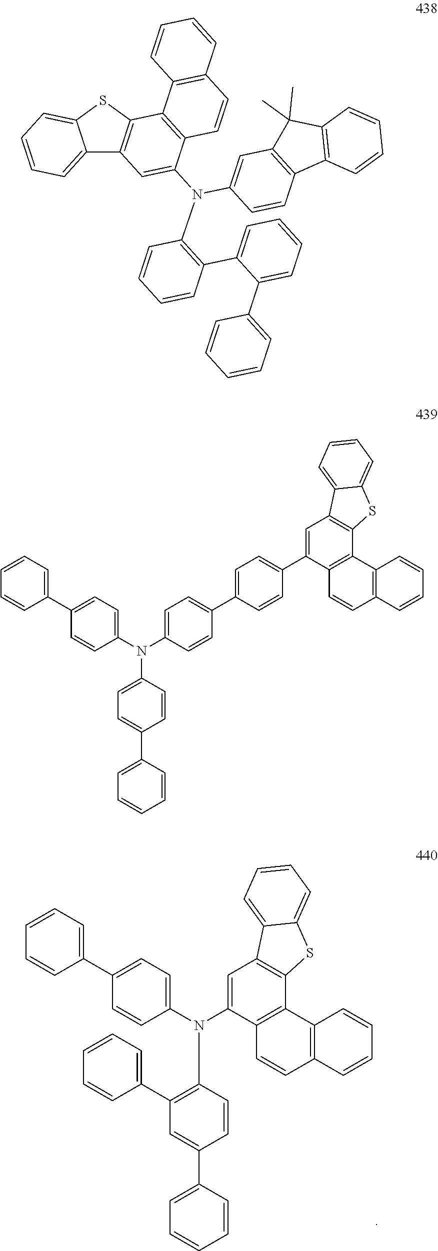 [SMILES: CC1(C)c2ccccc2-c2ccc(N(c3ccccc3-c3ccccc3-c3ccccc3)c3cc4c5ccccc5sc4c4c3ccc3ccccc34)cc21 ; c1ccc(-c2ccc(N(c3ccc(-c4ccccc4)cc3)c3ccc(-c4ccc(-c5cc6c7ccccc7sc6c6c5ccc5ccccc56)cc4)cc3)cc2)cc1 ; c1ccc(-c2ccc(N(c3ccc(-c4ccccc4)cc3-c3ccccc3)c3cc4c5ccccc5sc4c4c3ccc3ccccc34)cc2)cc1]

7. An organic light emitting device comprising:
a first electrode;
a second electrode provided opposite to the first electrode; and
one or more organic material layers provided between the first electrode and the second electrode,
wherein one or more layers of the one or more organic material layers comprise the heterocyclic compound of claim 1.

8. The organic light emitting device of claim 7, wherein the organic material layer comprises a hole transport layer, and the hole transport layer comprises the heterocyclic compound.

9. The organic light emitting device of claim 7, wherein the organic material layer comprises an electron blocking layer, and the electron blocking layer comprises the heterocyclic compound.

10. The organic light emitting device of claim 7, wherein the organic material layer comprises a light emitting layer, and the light emitting layer comprises the heterocyclic compound.

11. The organic light emitting device of claim 7, wherein the organic material layer further comprises a heterocyclic compound represented by the following Chemical Formula 2: 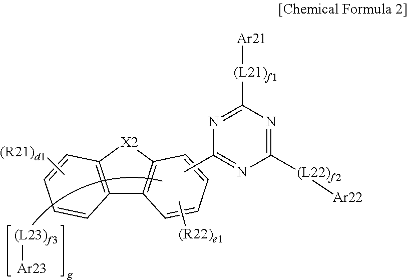
in Chemical Formula 2,
X2 is O; or S;
R21 and R22 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; —P(═O)R201R202; —SiR201R202R203; and —NR201R202, or two or more groups adjacent to each other bond to each other to form a substituted or unsubstituted C6 to C60 aromatic hydrocarbon ring; or a substituted or unsubstituted C2 to C60 heteroring, and R201, R202 and R203 are the same as or different from each other and each independently a substituted or unsubstituted C1 to C60 alkyl group; a substituted or unsubstituted C6 to C60 aryl group; or a substituted or unsubstituted C2 to C60 heteroaryl group;
d1 is an integer of 0 to 4, and when d1 is 2 or greater, R21s are the same as or different from each other;
e1 is an integer of 0 to 3, and when e1 is 2 or greater, R22s are the same as or different from each other;
L21 to L23 are the same as or different from each other, and each independently a direct bond; a substituted or unsubstituted C6 to C60 arylene group; or a substituted or unsubstituted C2 to C60 heteroarylene group;
f1 is an integer of 0 to 5, and when f1 is 2 or greater, L21s are the same as or different from each other;
f2 is an integer of 0 to 5, and when f2 is 2 or greater, L22s are the same as or different from each other;
f3 is an integer of 0 to 5, and when f3 is 2 or greater, L23s are the same as or different from each other;
Ar21 to Ar23 are the same as or different from each other, and each independently a substituted or unsubstituted C6 to C60 aryl group; a substituted or unsubstituted C2 to C60 heteroaryl group; or —NAr24Ar25;
Ar24 and Ar25 are the same as or different from each other, and each independently a substituted or unsubstituted C6 to C60 aryl group; or a substituted or unsubstituted C2 to C60 heteroaryl group; and
g is 0 or 1.

12. The organic light emitting device of claim 11, wherein the heterocyclic compound represented by Chemical Formula 2 is represented by any one of the following Chemical Formulae 2-1 to 2-3: 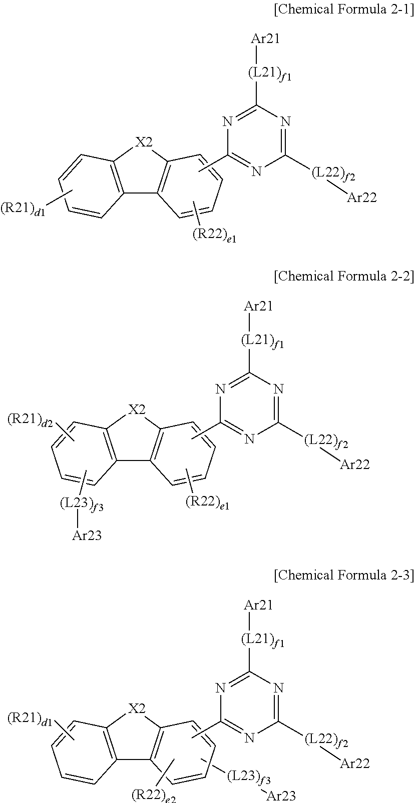
in Chemical Formulae 2-1 to 2-3,
d2 is an integer of 0 to 3, and when d2 is 2 or greater, R21s are the same as or different from each other;
e2 is an integer of 0 to 2, and when e2 is 2 or greater, R22s are the same as or different from each other; and
X2, R21, R22, L21 to L23, Ar21 to Ar23, d1, e1, and f1 to f3 have the same definitions as in Chemical Formula 2.

13. The organic light emitting device of claim 11, wherein the heterocyclic compound represented by Chemical Formula 2 does not include deuterium as a substituent, or has a deuterium content of 1% to 100% with respect to a total number of hydrogen atoms and deuterium atoms.

14. The organic light emitting device of claim 11, wherein the heterocyclic compound represented by Chemical Formula 2 is represented by any one of the following compounds:

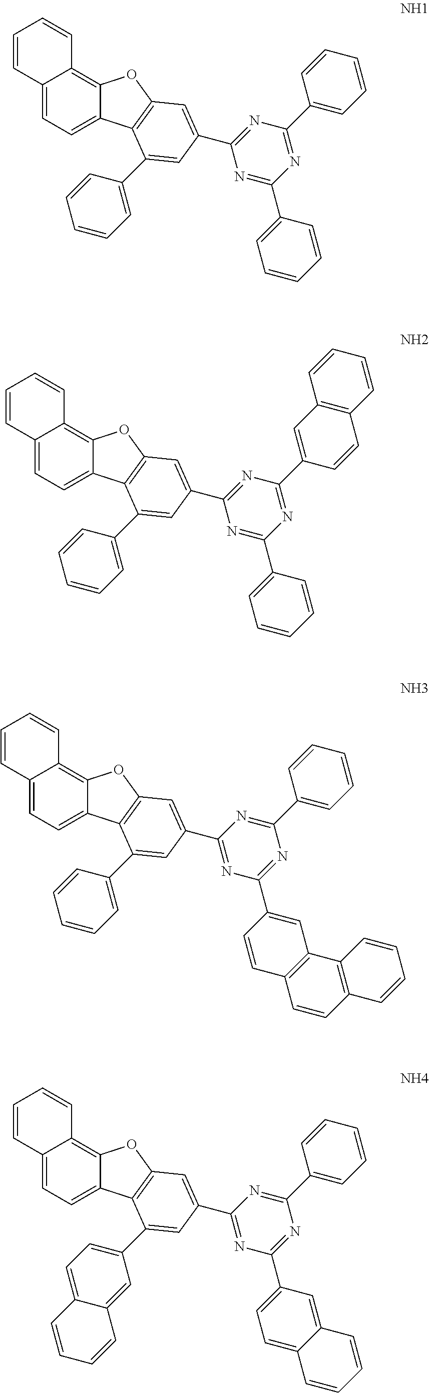 [SMILES: c1ccc(-c2nc(-c3ccccc3)nc(-c3cc(-c4ccccc4)c4c(c3)oc3c5ccccc5ccc34)n2)cc1 ; c1ccc(-c2nc(-c3ccc4ccccc4c3)nc(-c3cc(-c4ccccc4)c4c(c3)oc3c5ccccc5ccc34)n2)cc1 ; c1ccc(-c2nc(-c3cc(-c4ccccc4)c4c(c3)oc3c5ccccc5ccc34)nc(-c3ccc4ccc5ccccc5c4c3)n2)cc1 ; c1ccc(-c2nc(-c3ccc4ccccc4c3)nc(-c3cc(-c4ccc5ccccc5c4)c4c(c3)oc3c5ccccc5ccc34)n2)cc1]

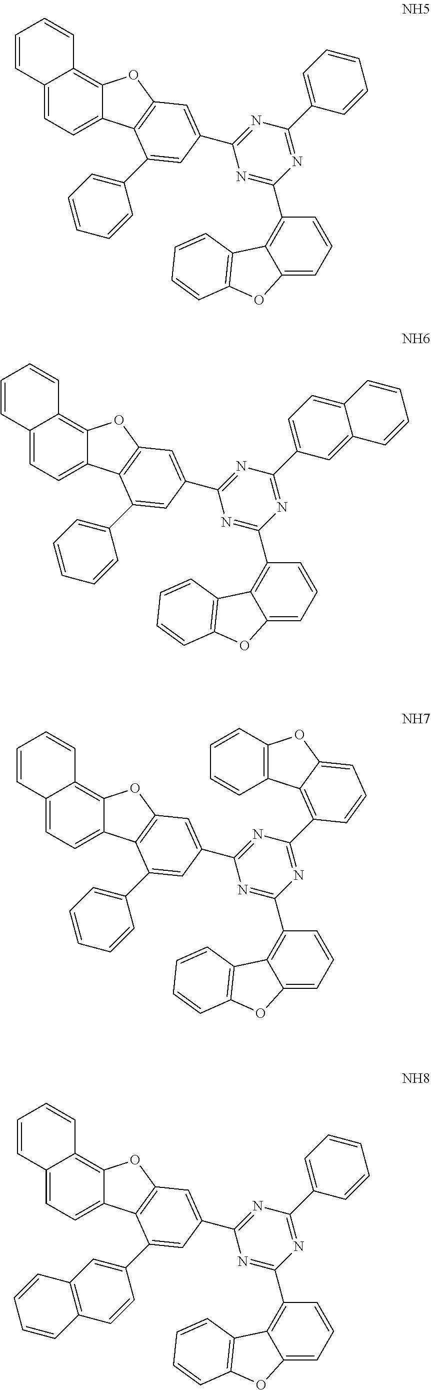 [SMILES: c1ccc(-c2nc(-c3cc(-c4ccccc4)c4c(c3)oc3c5ccccc5ccc34)nc(-c3cccc4oc5ccccc5c34)n2)cc1 ; c1ccc(-c2cc(-c3nc(-c4ccc5ccccc5c4)nc(-c4cccc5oc6ccccc6c45)n3)cc3oc4c5ccccc5ccc4c23)cc1 ; c1ccc(-c2cc(-c3nc(-c4cccc5oc6ccccc6c45)nc(-c4cccc5oc6ccccc6c45)n3)cc3oc4c5ccccc5ccc4c23)cc1 ; c1ccc(-c2nc(-c3cc(-c4ccc5ccccc5c4)c4c(c3)oc3c5ccccc5ccc34)nc(-c3cccc4oc5ccccc5c34)n2)cc1]

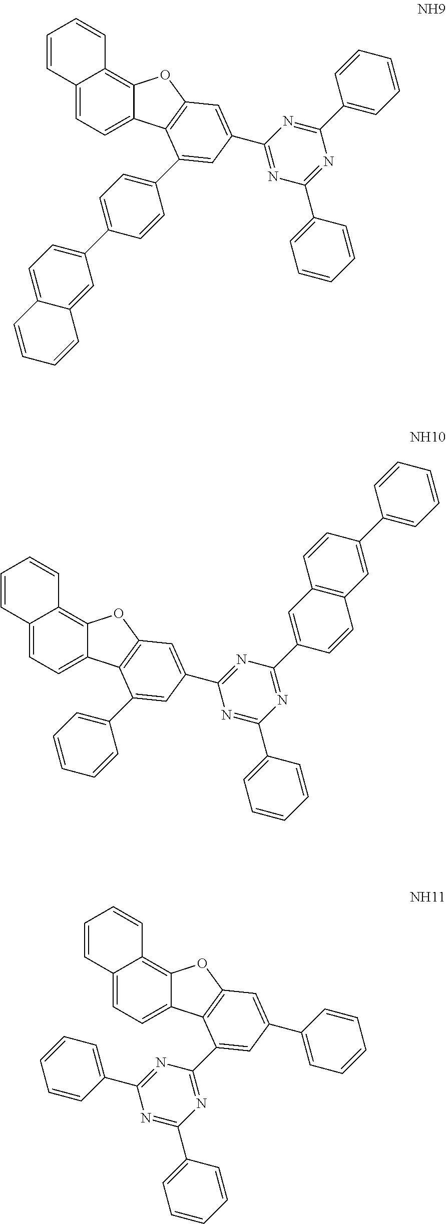 [SMILES: c1ccc(-c2nc(-c3ccccc3)nc(-c3cc(-c4ccc(-c5ccc6ccccc6c5)cc4)c4c(c3)oc3c5ccccc5ccc34)n2)cc1 ; c1ccc(-c2ccc3cc(-c4nc(-c5ccccc5)nc(-c5cc(-c6ccccc6)c6c(c5)oc5c7ccccc7ccc56)n4)ccc3c2)cc1 ; c1ccc(-c2cc(-c3nc(-c4ccccc4)nc(-c4ccccc4)n3)c3c(c2)oc2c4ccccc4ccc23)cc1]

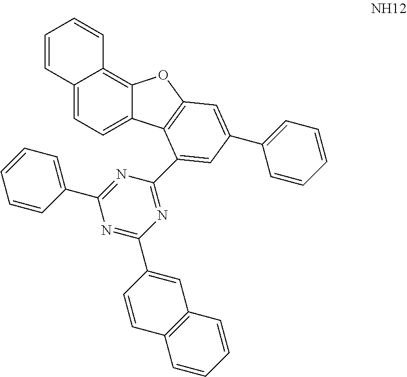 [SMILES: c1ccc(-c2cc(-c3nc(-c4ccccc4)nc(-c4ccc5ccccc5c4)n3)c3c(c2)oc2c4ccccc4ccc23)cc1]

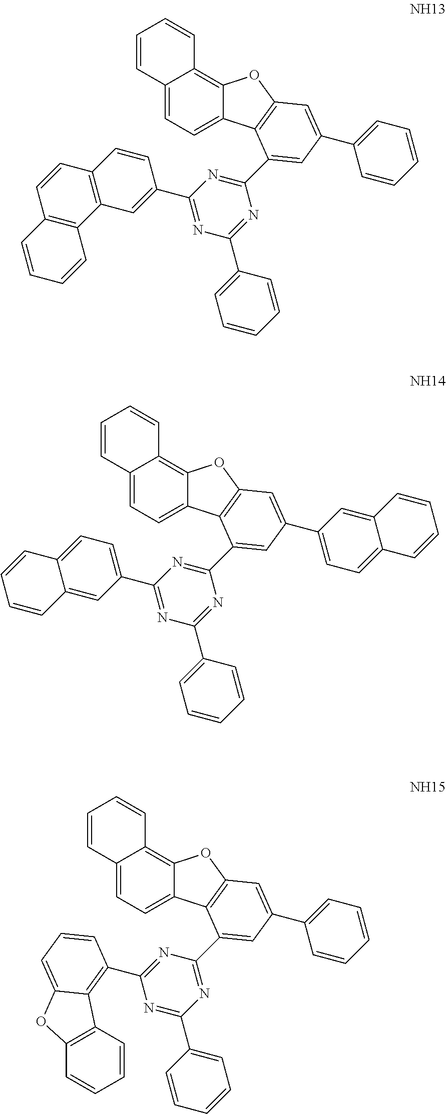 [SMILES: c1ccc(-c2cc(-c3nc(-c4ccccc4)nc(-c4ccc5ccc6ccccc6c5c4)n3)c3c(c2)oc2c4ccccc4ccc23)cc1 ; c1ccc(-c2nc(-c3ccc4ccccc4c3)nc(-c3cc(-c4ccc5ccccc5c4)cc4oc5c6ccccc6ccc5c34)n2)cc1 ; c1ccc(-c2cc(-c3nc(-c4ccccc4)nc(-c4cccc5oc6ccccc6c45)n3)c3c(c2)oc2c4ccccc4ccc23)cc1]

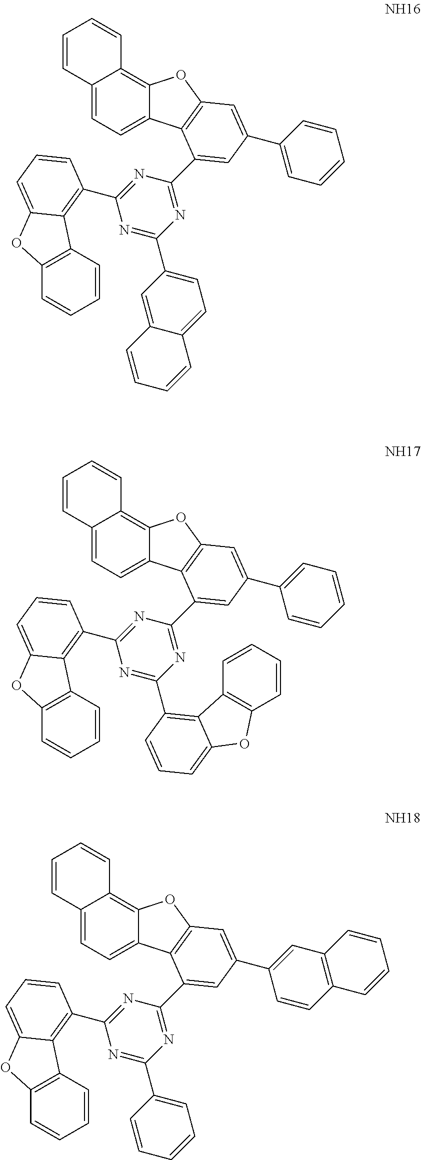 [SMILES: c1ccc(-c2cc(-c3nc(-c4ccc5ccccc5c4)nc(-c4cccc5oc6ccccc6c45)n3)c3c(c2)oc2c4ccccc4ccc23)cc1 ; c1ccc(-c2cc(-c3nc(-c4cccc5oc6ccccc6c45)nc(-c4cccc5oc6ccccc6c45)n3)c3c(c2)oc2c4ccccc4ccc23)cc1 ; c1ccc(-c2nc(-c3cccc4oc5ccccc5c34)nc(-c3cc(-c4ccc5ccccc5c4)cc4oc5c6ccccc6ccc5c34)n2)cc1]

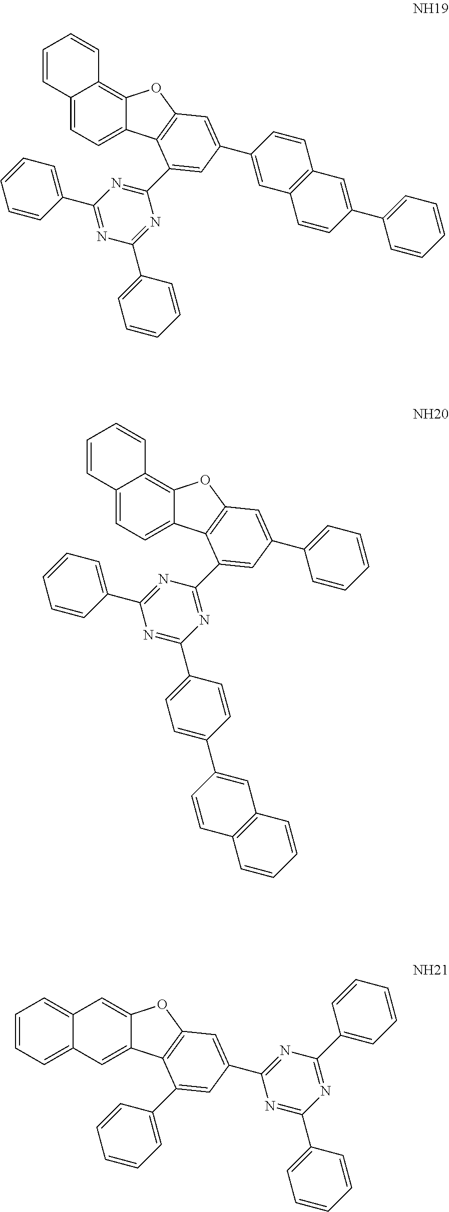 [SMILES: c1ccc(-c2ccc3cc(-c4cc(-c5nc(-c6ccccc6)nc(-c6ccccc6)n5)c5c(c4)oc4c6ccccc6ccc45)ccc3c2)cc1 ; c1ccc(-c2cc(-c3nc(-c4ccccc4)nc(-c4ccc(-c5ccc6ccccc6c5)cc4)n3)c3c(c2)oc2c4ccccc4ccc23)cc1 ; c1ccc(-c2nc(-c3ccccc3)nc(-c3cc(-c4ccccc4)c4c(c3)oc3cc5ccccc5cc34)n2)cc1]

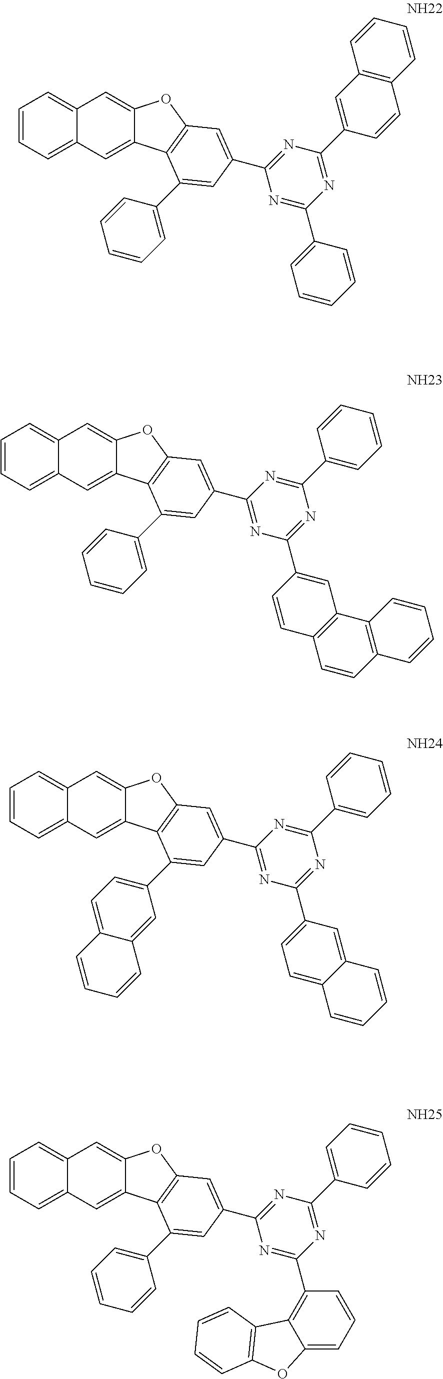 [SMILES: c1ccc(-c2nc(-c3ccc4ccccc4c3)nc(-c3cc(-c4ccccc4)c4c(c3)oc3cc5ccccc5cc34)n2)cc1 ; c1ccc(-c2nc(-c3cc(-c4ccccc4)c4c(c3)oc3cc5ccccc5cc34)nc(-c3ccc4ccc5ccccc5c4c3)n2)cc1 ; c1ccc(-c2nc(-c3ccc4ccccc4c3)nc(-c3cc(-c4ccc5ccccc5c4)c4c(c3)oc3cc5ccccc5cc34)n2)cc1 ; c1ccc(-c2nc(-c3cc(-c4ccccc4)c4c(c3)oc3cc5ccccc5cc34)nc(-c3cccc4oc5ccccc5c34)n2)cc1]

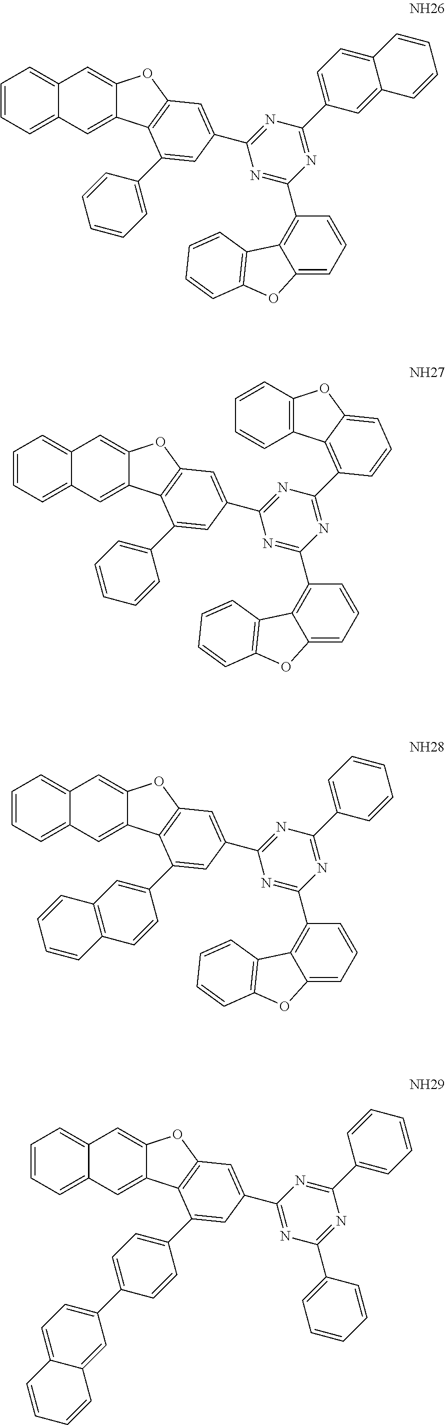 [SMILES: c1ccc(-c2cc(-c3nc(-c4ccc5ccccc5c4)nc(-c4cccc5oc6ccccc6c45)n3)cc3oc4cc5ccccc5cc4c23)cc1 ; c1ccc(-c2cc(-c3nc(-c4cccc5oc6ccccc6c45)nc(-c4cccc5oc6ccccc6c45)n3)cc3oc4cc5ccccc5cc4c23)cc1 ; c1ccc(-c2nc(-c3cc(-c4ccc5ccccc5c4)c4c(c3)oc3cc5ccccc5cc34)nc(-c3cccc4oc5ccccc5c34)n2)cc1 ; c1ccc(-c2nc(-c3ccccc3)nc(-c3cc(-c4ccc(-c5ccc6ccccc6c5)cc4)c4c(c3)oc3cc5ccccc5cc34)n2)cc1]

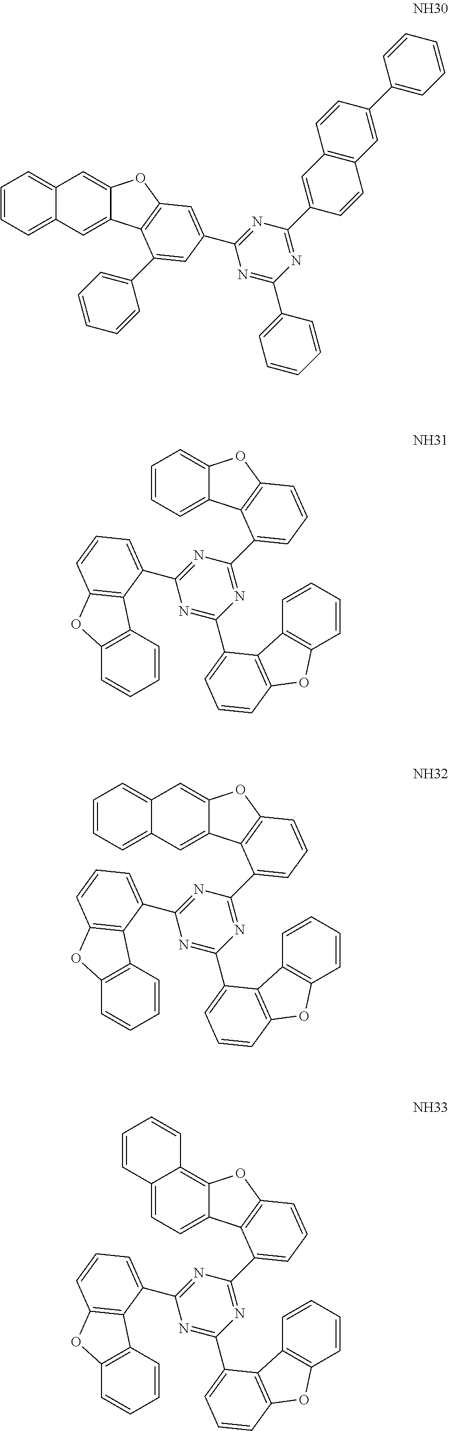 [SMILES: c1ccc(-c2ccc3cc(-c4nc(-c5ccccc5)nc(-c5cc(-c6ccccc6)c6c(c5)oc5cc7ccccc7cc56)n4)ccc3c2)cc1 ; c1ccc2c(c1)oc1cccc(-c3nc(-c4cccc5oc6ccccc6c45)nc(-c4cccc5oc6ccccc6c45)n3)c12 ; c1ccc2cc3c(cc2c1)oc1cccc(-c2nc(-c4cccc5oc6ccccc6c45)nc(-c4cccc5oc6ccccc6c45)n2)c13 ; c1ccc2c(c1)ccc1c2oc2cccc(-c3nc(-c4cccc5oc6ccccc6c45)nc(-c4cccc5oc6ccccc6c45)n3)c21]

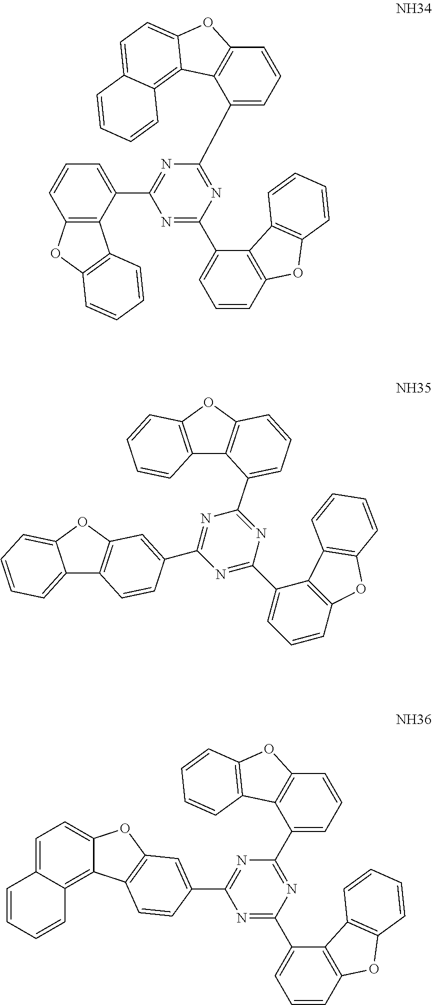 [SMILES: c1ccc2c(c1)ccc1oc3cccc(-c4nc(-c5cccc6oc7ccccc7c56)nc(-c5cccc6oc7ccccc7c56)n4)c3c12 ; c1ccc2c(c1)oc1cc(-c3nc(-c4cccc5oc6ccccc6c45)nc(-c4cccc5oc6ccccc6c45)n3)ccc12 ; c1ccc2c(c1)ccc1oc3cc(-c4nc(-c5cccc6oc7ccccc7c56)nc(-c5cccc6oc7ccccc7c56)n4)ccc3c12]

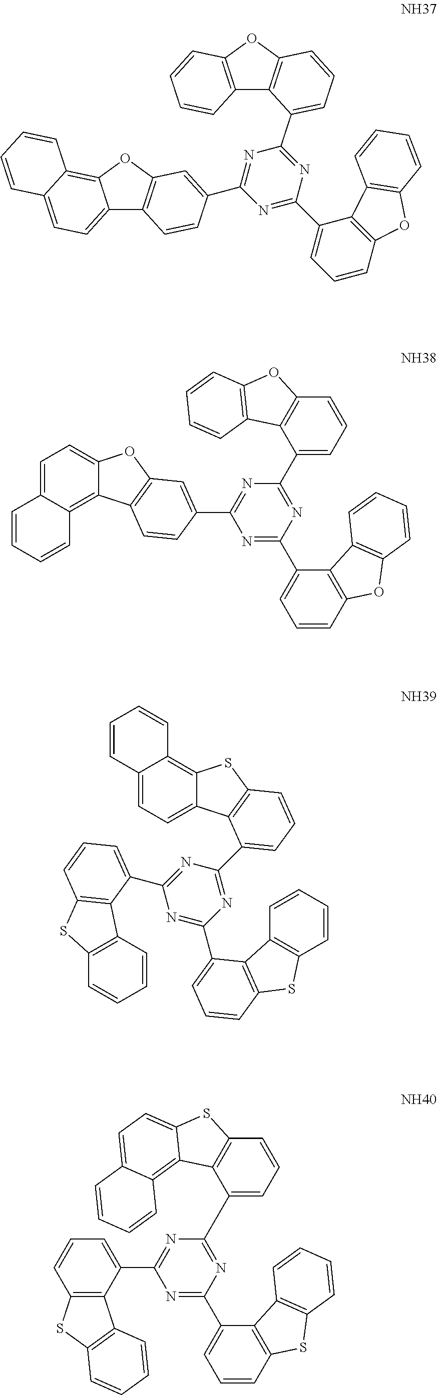 [SMILES: c1ccc2c(c1)ccc1c3ccc(-c4nc(-c5cccc6oc7ccccc7c56)nc(-c5cccc6oc7ccccc7c56)n4)cc3oc21 ; c1ccc2c(c1)ccc1oc3cc(-c4nc(-c5cccc6oc7ccccc7c56)nc(-c5cccc6oc7ccccc7c56)n4)ccc3c12 ; c1ccc2c(c1)ccc1c2sc2cccc(-c3nc(-c4cccc5sc6ccccc6c45)nc(-c4cccc5sc6ccccc6c45)n3)c21 ; c1ccc2c(c1)ccc1sc3cccc(-c4nc(-c5cccc6sc7ccccc7c56)nc(-c5cccc6sc7ccccc7c56)n4)c3c12]

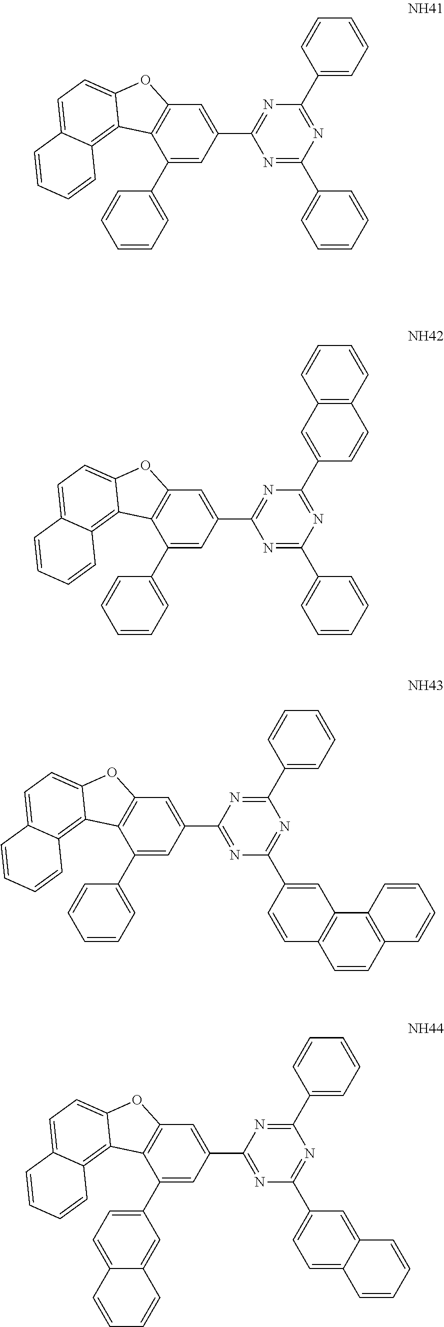 [SMILES: c1ccc(-c2nc(-c3ccccc3)nc(-c3cc(-c4ccccc4)c4c(c3)oc3ccc5ccccc5c34)n2)cc1 ; c1ccc(-c2nc(-c3ccc4ccccc4c3)nc(-c3cc(-c4ccccc4)c4c(c3)oc3ccc5ccccc5c34)n2)cc1 ; c1ccc(-c2nc(-c3cc(-c4ccccc4)c4c(c3)oc3ccc5ccccc5c34)nc(-c3ccc4ccc5ccccc5c4c3)n2)cc1 ; c1ccc(-c2nc(-c3ccc4ccccc4c3)nc(-c3cc(-c4ccc5ccccc5c4)c4c(c3)oc3ccc5ccccc5c34)n2)cc1]

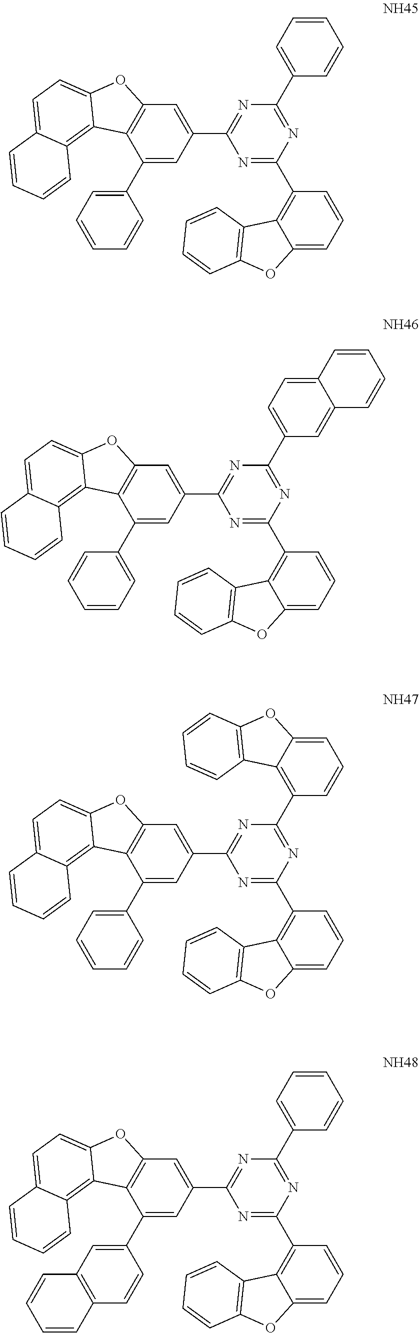 [SMILES: c1ccc(-c2nc(-c3cc(-c4ccccc4)c4c(c3)oc3ccc5ccccc5c34)nc(-c3cccc4oc5ccccc5c34)n2)cc1 ; c1ccc(-c2cc(-c3nc(-c4ccc5ccccc5c4)nc(-c4cccc5oc6ccccc6c45)n3)cc3oc4ccc5ccccc5c4c23)cc1 ; c1ccc(-c2cc(-c3nc(-c4cccc5oc6ccccc6c45)nc(-c4cccc5oc6ccccc6c45)n3)cc3oc4ccc5ccccc5c4c23)cc1 ; c1ccc(-c2nc(-c3cc(-c4ccc5ccccc5c4)c4c(c3)oc3ccc5ccccc5c34)nc(-c3cccc4oc5ccccc5c34)n2)cc1]

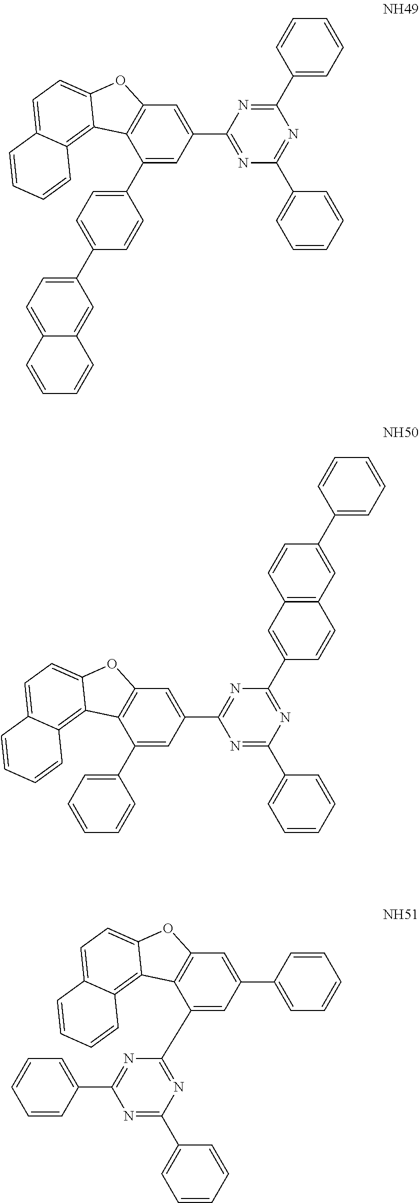 [SMILES: c1ccc(-c2nc(-c3ccccc3)nc(-c3cc(-c4ccc(-c5ccc6ccccc6c5)cc4)c4c(c3)oc3ccc5ccccc5c34)n2)cc1 ; c1ccc(-c2ccc3cc(-c4nc(-c5ccccc5)nc(-c5cc(-c6ccccc6)c6c(c5)oc5ccc7ccccc7c56)n4)ccc3c2)cc1 ; c1ccc(-c2cc(-c3nc(-c4ccccc4)nc(-c4ccccc4)n3)c3c(c2)oc2ccc4ccccc4c23)cc1]

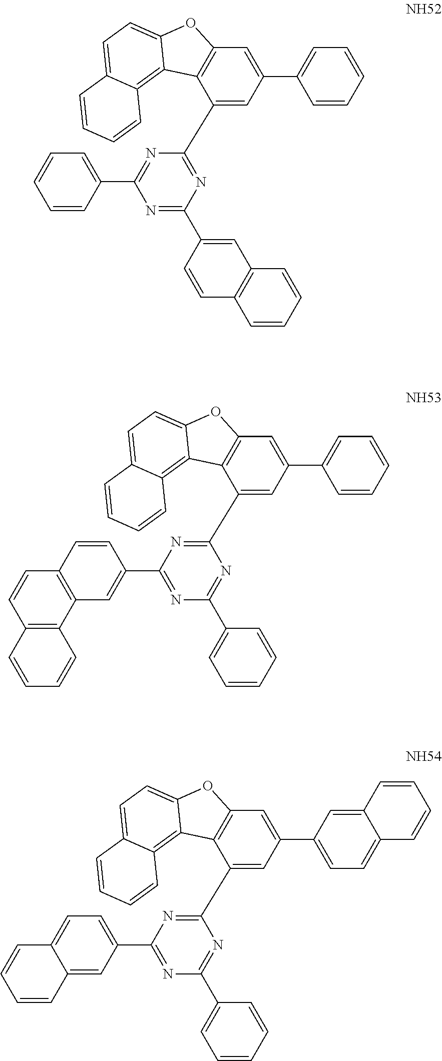 [SMILES: c1ccc(-c2cc(-c3nc(-c4ccccc4)nc(-c4ccc5ccccc5c4)n3)c3c(c2)oc2ccc4ccccc4c23)cc1 ; c1ccc(-c2cc(-c3nc(-c4ccccc4)nc(-c4ccc5ccc6ccccc6c5c4)n3)c3c(c2)oc2ccc4ccccc4c23)cc1 ; c1ccc(-c2nc(-c3ccc4ccccc4c3)nc(-c3cc(-c4ccc5ccccc5c4)cc4oc5ccc6ccccc6c5c34)n2)cc1]

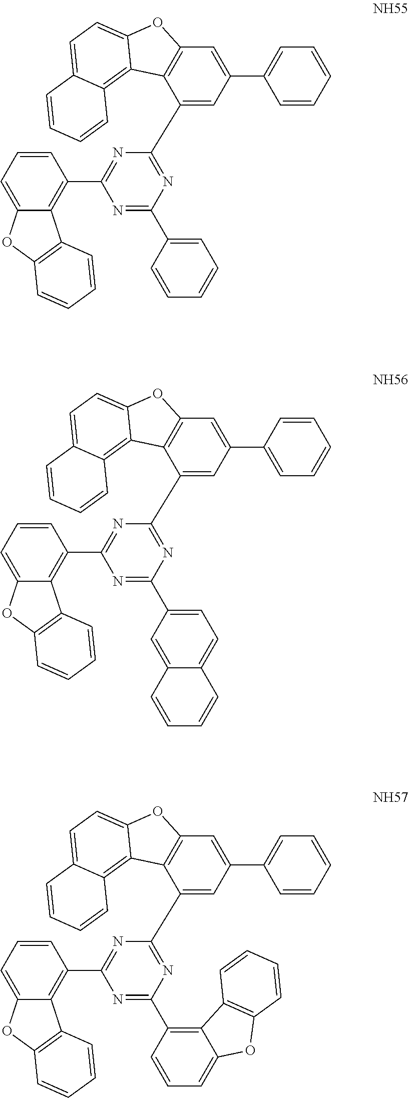 [SMILES: c1ccc(-c2cc(-c3nc(-c4ccccc4)nc(-c4cccc5oc6ccccc6c45)n3)c3c(c2)oc2ccc4ccccc4c23)cc1 ; c1ccc(-c2cc(-c3nc(-c4ccc5ccccc5c4)nc(-c4cccc5oc6ccccc6c45)n3)c3c(c2)oc2ccc4ccccc4c23)cc1 ; c1ccc(-c2cc(-c3nc(-c4cccc5oc6ccccc6c45)nc(-c4cccc5oc6ccccc6c45)n3)c3c(c2)oc2ccc4ccccc4c23)cc1]

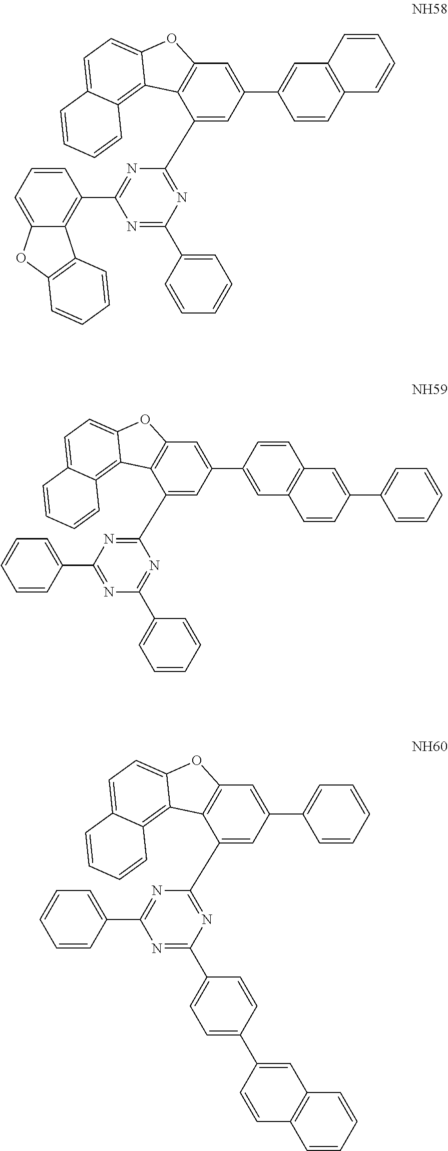 [SMILES: c1ccc(-c2nc(-c3cccc4oc5ccccc5c34)nc(-c3cc(-c4ccc5ccccc5c4)cc4oc5ccc6ccccc6c5c34)n2)cc1 ; c1ccc(-c2ccc3cc(-c4cc(-c5nc(-c6ccccc6)nc(-c6ccccc6)n5)c5c(c4)oc4ccc6ccccc6c45)ccc3c2)cc1 ; c1ccc(-c2cc(-c3nc(-c4ccccc4)nc(-c4ccc(-c5ccc6ccccc6c5)cc4)n3)c3c(c2)oc2ccc4ccccc4c23)cc1]

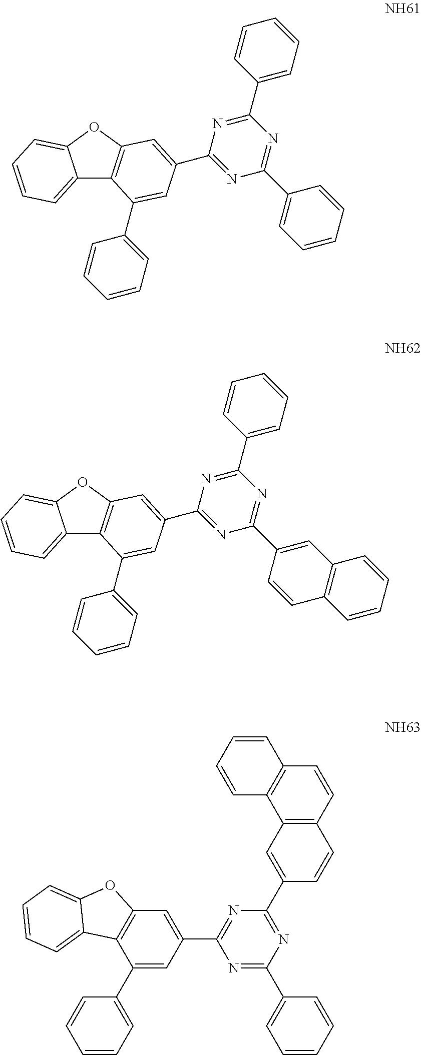 [SMILES: c1ccc(-c2nc(-c3ccccc3)nc(-c3cc(-c4ccccc4)c4c(c3)oc3ccccc34)n2)cc1 ; c1ccc(-c2nc(-c3ccc4ccccc4c3)nc(-c3cc(-c4ccccc4)c4c(c3)oc3ccccc34)n2)cc1 ; c1ccc(-c2nc(-c3cc(-c4ccccc4)c4c(c3)oc3ccccc34)nc(-c3ccc4ccc5ccccc5c4c3)n2)cc1]

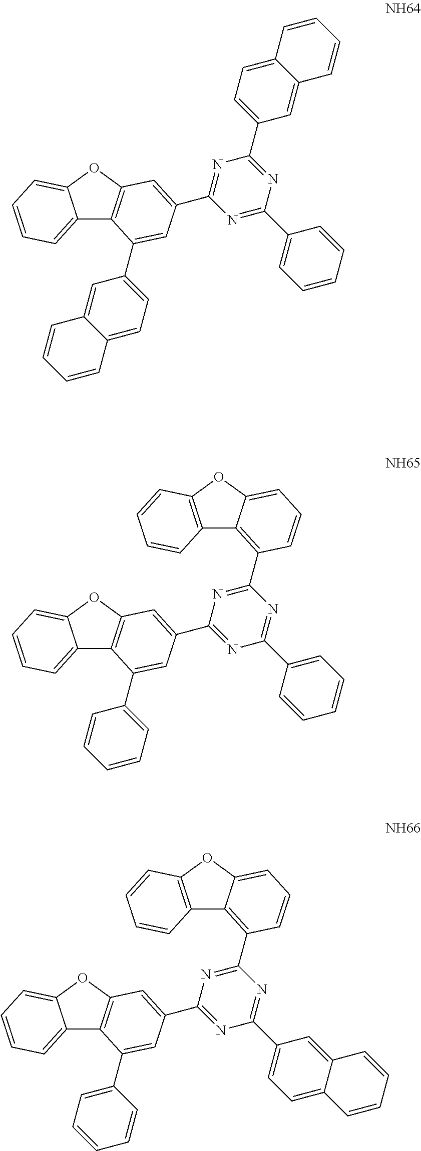 [SMILES: c1ccc(-c2nc(-c3ccc4ccccc4c3)nc(-c3cc(-c4ccc5ccccc5c4)c4c(c3)oc3ccccc34)n2)cc1 ; c1ccc(-c2nc(-c3cc(-c4ccccc4)c4c(c3)oc3ccccc34)nc(-c3cccc4oc5ccccc5c34)n2)cc1 ; c1ccc(-c2cc(-c3nc(-c4ccc5ccccc5c4)nc(-c4cccc5oc6ccccc6c45)n3)cc3oc4ccccc4c23)cc1]

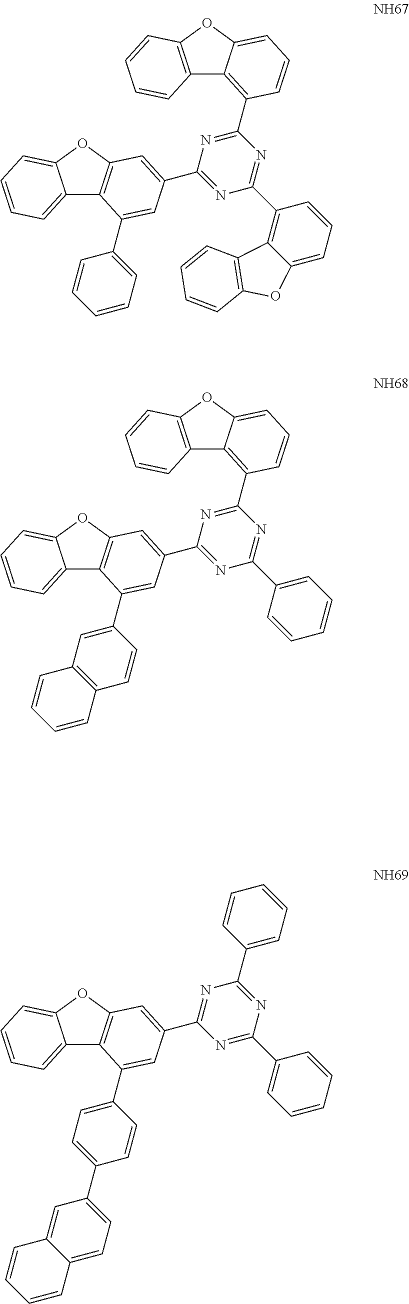 [SMILES: c1ccc(-c2cc(-c3nc(-c4cccc5oc6ccccc6c45)nc(-c4cccc5oc6ccccc6c45)n3)cc3oc4ccccc4c23)cc1 ; c1ccc(-c2nc(-c3cc(-c4ccc5ccccc5c4)c4c(c3)oc3ccccc34)nc(-c3cccc4oc5ccccc5c34)n2)cc1 ; c1ccc(-c2nc(-c3ccccc3)nc(-c3cc(-c4ccc(-c5ccc6ccccc6c5)cc4)c4c(c3)oc3ccccc34)n2)cc1]

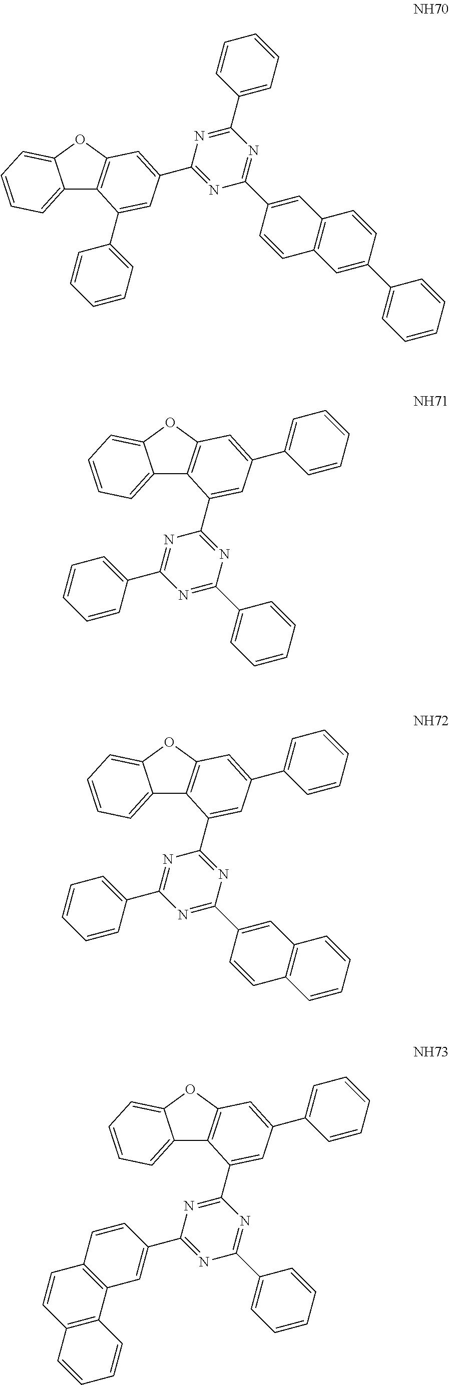 [SMILES: c1ccc(-c2ccc3cc(-c4nc(-c5ccccc5)nc(-c5cc(-c6ccccc6)c6c(c5)oc5ccccc56)n4)ccc3c2)cc1 ; c1ccc(-c2cc(-c3nc(-c4ccccc4)nc(-c4ccccc4)n3)c3c(c2)oc2ccccc23)cc1 ; c1ccc(-c2cc(-c3nc(-c4ccccc4)nc(-c4ccc5ccccc5c4)n3)c3c(c2)oc2ccccc23)cc1 ; c1ccc(-c2cc(-c3nc(-c4ccccc4)nc(-c4ccc5ccc6ccccc6c5c4)n3)c3c(c2)oc2ccccc23)cc1]

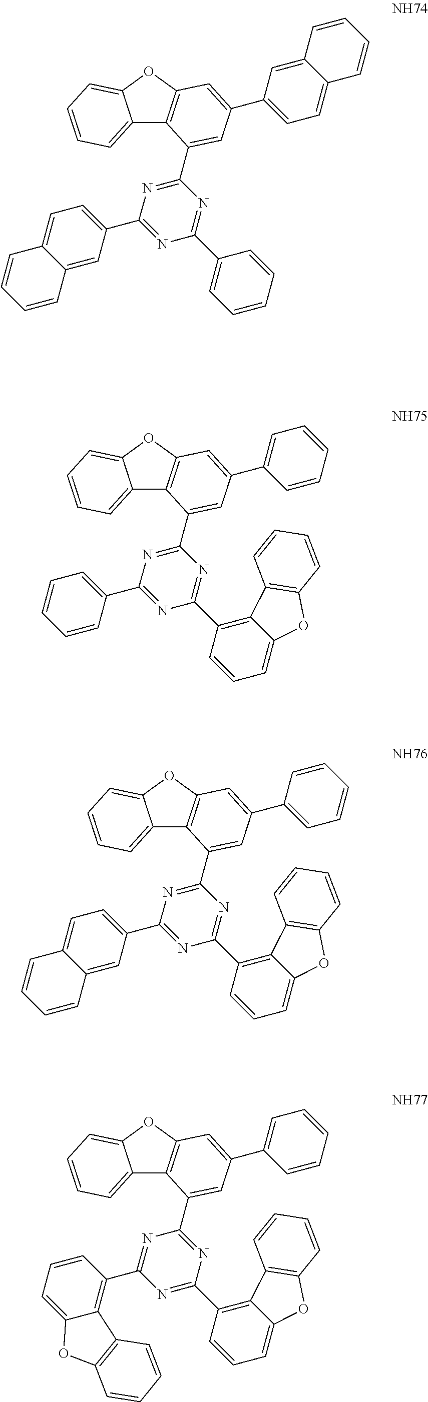 [SMILES: c1ccc(-c2nc(-c3ccc4ccccc4c3)nc(-c3cc(-c4ccc5ccccc5c4)cc4oc5ccccc5c34)n2)cc1 ; c1ccc(-c2cc(-c3nc(-c4ccccc4)nc(-c4cccc5oc6ccccc6c45)n3)c3c(c2)oc2ccccc23)cc1 ; c1ccc(-c2cc(-c3nc(-c4ccc5ccccc5c4)nc(-c4cccc5oc6ccccc6c45)n3)c3c(c2)oc2ccccc23)cc1 ; c1ccc(-c2cc(-c3nc(-c4cccc5oc6ccccc6c45)nc(-c4cccc5oc6ccccc6c45)n3)c3c(c2)oc2ccccc23)cc1]

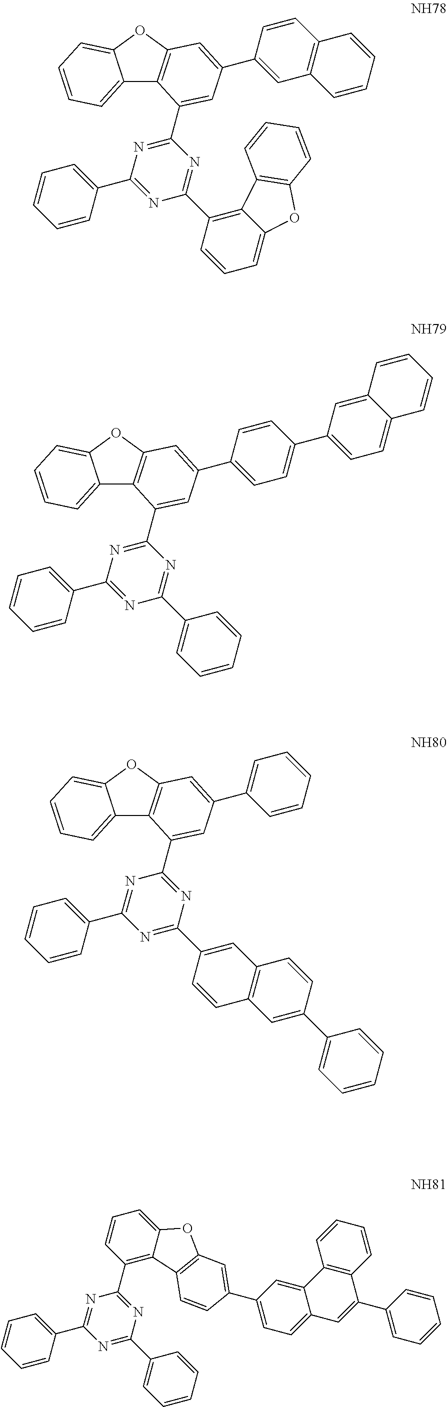 [SMILES: c1ccc(-c2nc(-c3cccc4oc5ccccc5c34)nc(-c3cc(-c4ccc5ccccc5c4)cc4oc5ccccc5c34)n2)cc1 ; c1ccc(-c2nc(-c3ccccc3)nc(-c3cc(-c4ccc(-c5ccc6ccccc6c5)cc4)cc4oc5ccccc5c34)n2)cc1 ; c1ccc(-c2ccc3cc(-c4nc(-c5ccccc5)nc(-c5cc(-c6ccccc6)cc6oc7ccccc7c56)n4)ccc3c2)cc1 ; c1ccc(-c2nc(-c3ccccc3)nc(-c3cccc4oc5cc(-c6ccc7cc(-c8ccccc8)c8ccccc8c7c6)ccc5c34)n2)cc1]

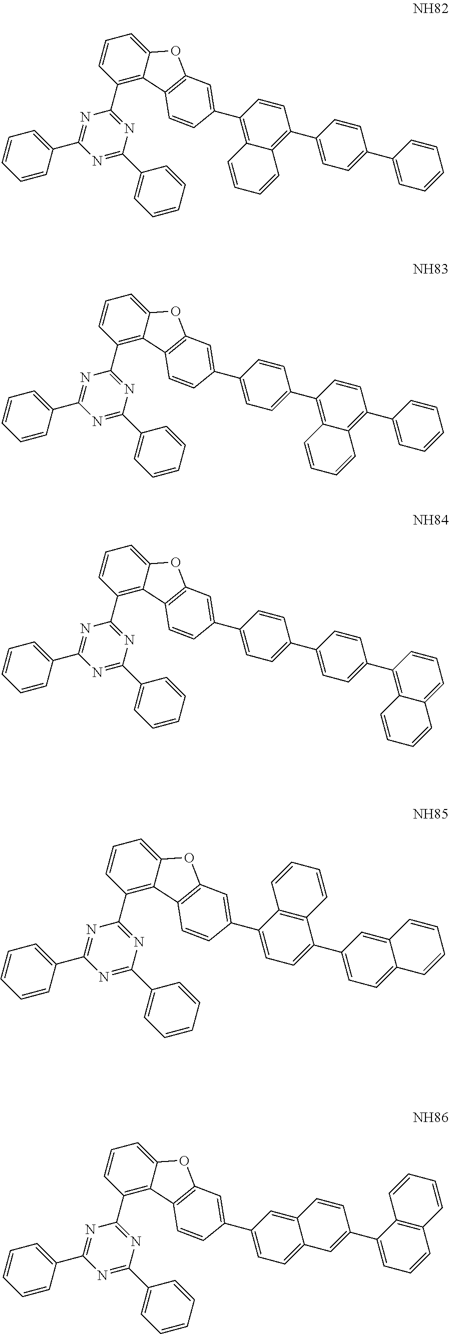 [SMILES: c1ccc(-c2ccc(-c3ccc(-c4ccc5c(c4)oc4cccc(-c6nc(-c7ccccc7)nc(-c7ccccc7)n6)c45)c4ccccc34)cc2)cc1 ; c1ccc(-c2nc(-c3ccccc3)nc(-c3cccc4oc5cc(-c6ccc(-c7ccc(-c8ccccc8)c8ccccc78)cc6)ccc5c34)n2)cc1 ; c1ccc(-c2nc(-c3ccccc3)nc(-c3cccc4oc5cc(-c6ccc(-c7ccc(-c8cccc9ccccc89)cc7)cc6)ccc5c34)n2)cc1 ; c1ccc(-c2nc(-c3ccccc3)nc(-c3cccc4oc5cc(-c6ccc(-c7ccc8ccccc8c7)c7ccccc67)ccc5c34)n2)cc1 ; c1ccc(-c2nc(-c3ccccc3)nc(-c3cccc4oc5cc(-c6ccc7cc(-c8cccc9ccccc89)ccc7c6)ccc5c34)n2)cc1]

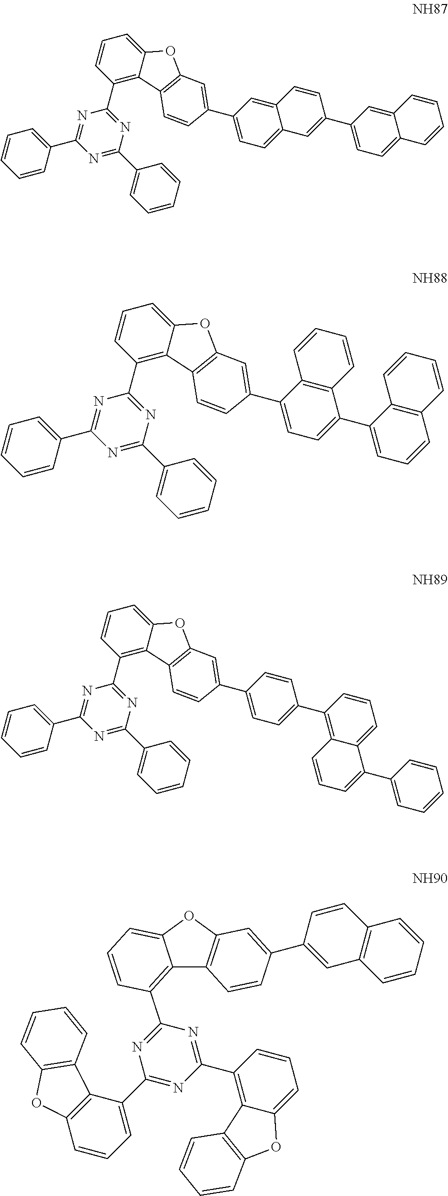 [SMILES: c1ccc(-c2nc(-c3ccccc3)nc(-c3cccc4oc5cc(-c6ccc7cc(-c8ccc9ccccc9c8)ccc7c6)ccc5c34)n2)cc1 ; c1ccc(-c2nc(-c3ccccc3)nc(-c3cccc4oc5cc(-c6ccc(-c7cccc8ccccc78)c7ccccc67)ccc5c34)n2)cc1 ; c1ccc(-c2nc(-c3ccccc3)nc(-c3cccc4oc5cc(-c6ccc(-c7cccc8c(-c9ccccc9)cccc78)cc6)ccc5c34)n2)cc1 ; c1ccc2cc(-c3ccc4c(c3)oc3cccc(-c5nc(-c6cccc7oc8ccccc8c67)nc(-c6cccc7oc8ccccc8c67)n5)c34)ccc2c1]

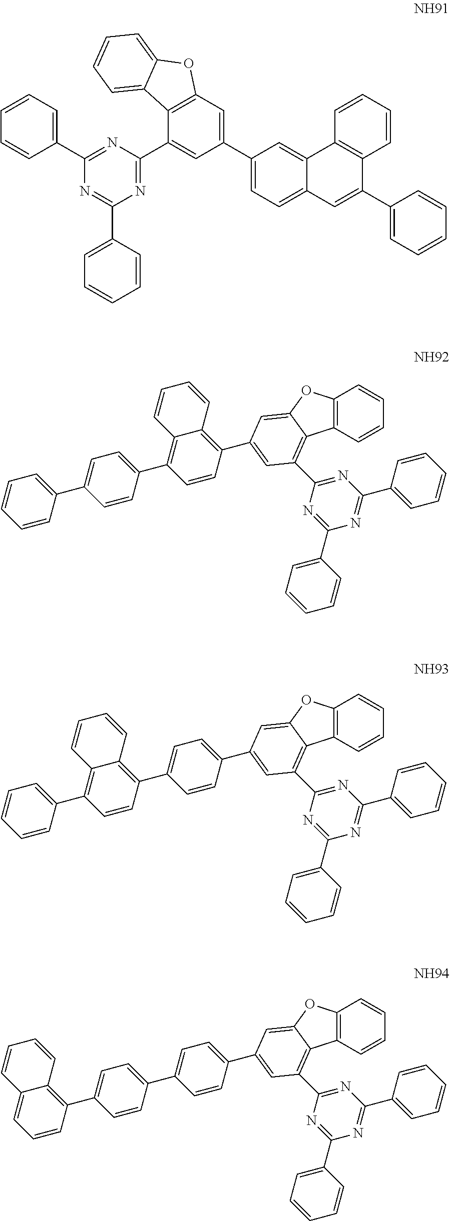 [SMILES: c1ccc(-c2nc(-c3ccccc3)nc(-c3cc(-c4ccc5cc(-c6ccccc6)c6ccccc6c5c4)cc4oc5ccccc5c34)n2)cc1 ; c1ccc(-c2ccc(-c3ccc(-c4cc(-c5nc(-c6ccccc6)nc(-c6ccccc6)n5)c5c(c4)oc4ccccc45)c4ccccc34)cc2)cc1 ; c1ccc(-c2nc(-c3ccccc3)nc(-c3cc(-c4ccc(-c5ccc(-c6ccccc6)c6ccccc56)cc4)cc4oc5ccccc5c34)n2)cc1 ; c1ccc(-c2nc(-c3ccccc3)nc(-c3cc(-c4ccc(-c5ccc(-c6cccc7ccccc67)cc5)cc4)cc4oc5ccccc5c34)n2)cc1]

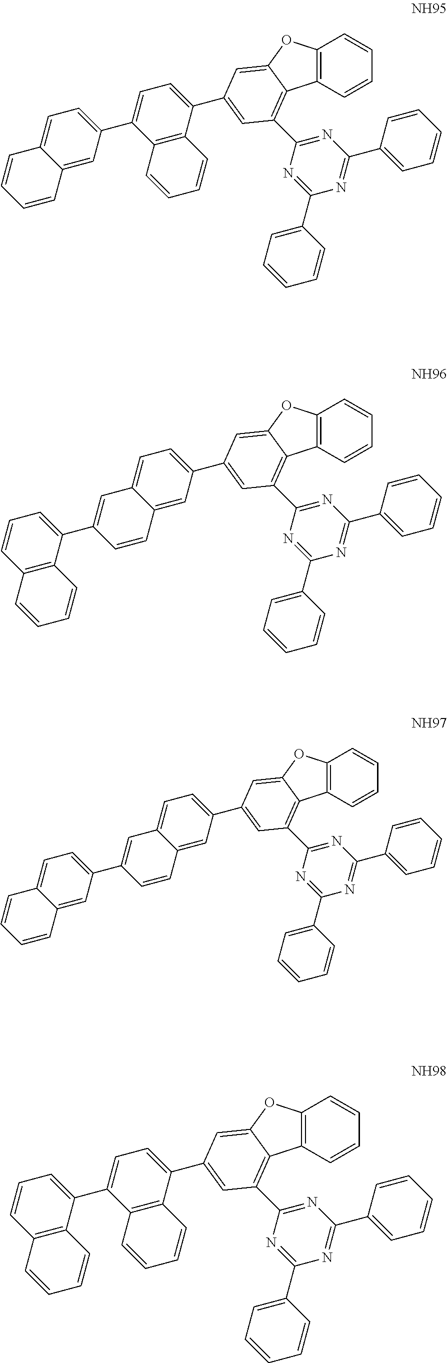 [SMILES: c1ccc(-c2nc(-c3ccccc3)nc(-c3cc(-c4ccc(-c5ccc6ccccc6c5)c5ccccc45)cc4oc5ccccc5c34)n2)cc1 ; c1ccc(-c2nc(-c3ccccc3)nc(-c3cc(-c4ccc5cc(-c6cccc7ccccc67)ccc5c4)cc4oc5ccccc5c34)n2)cc1 ; c1ccc(-c2nc(-c3ccccc3)nc(-c3cc(-c4ccc5cc(-c6ccc7ccccc7c6)ccc5c4)cc4oc5ccccc5c34)n2)cc1 ; c1ccc(-c2nc(-c3ccccc3)nc(-c3cc(-c4ccc(-c5cccc6ccccc56)c5ccccc45)cc4oc5ccccc5c34)n2)cc1]

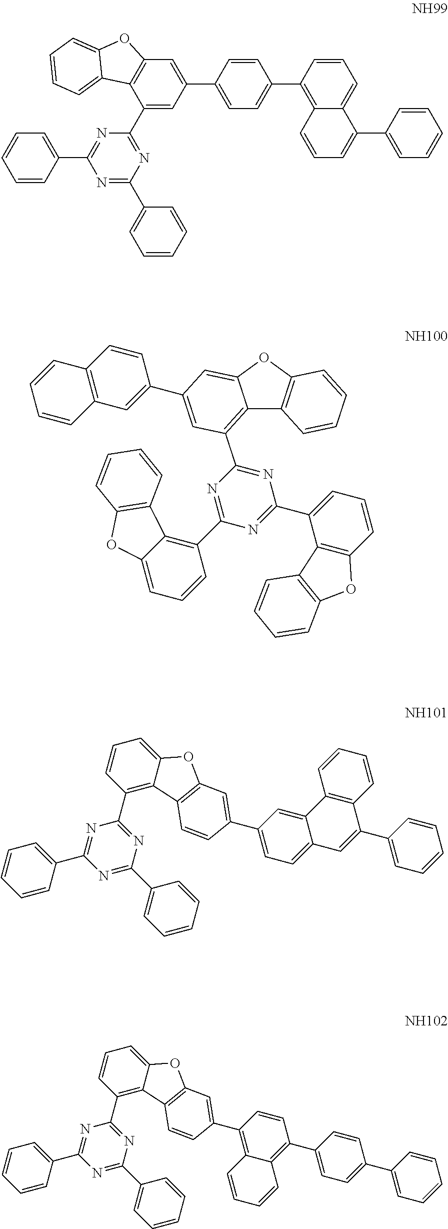 [SMILES: c1ccc(-c2nc(-c3ccccc3)nc(-c3cc(-c4ccc(-c5cccc6c(-c7ccccc7)cccc56)cc4)cc4oc5ccccc5c34)n2)cc1 ; c1ccc2cc(-c3cc(-c4nc(-c5cccc6oc7ccccc7c56)nc(-c5cccc6oc7ccccc7c56)n4)c4c(c3)oc3ccccc34)ccc2c1 ; c1ccc(-c2nc(-c3ccccc3)nc(-c3cccc4oc5cc(-c6ccc7cc(-c8ccccc8)c8ccccc8c7c6)ccc5c34)n2)cc1 ; c1ccc(-c2ccc(-c3ccc(-c4ccc5c(c4)oc4cccc(-c6nc(-c7ccccc7)nc(-c7ccccc7)n6)c45)c4ccccc34)cc2)cc1]

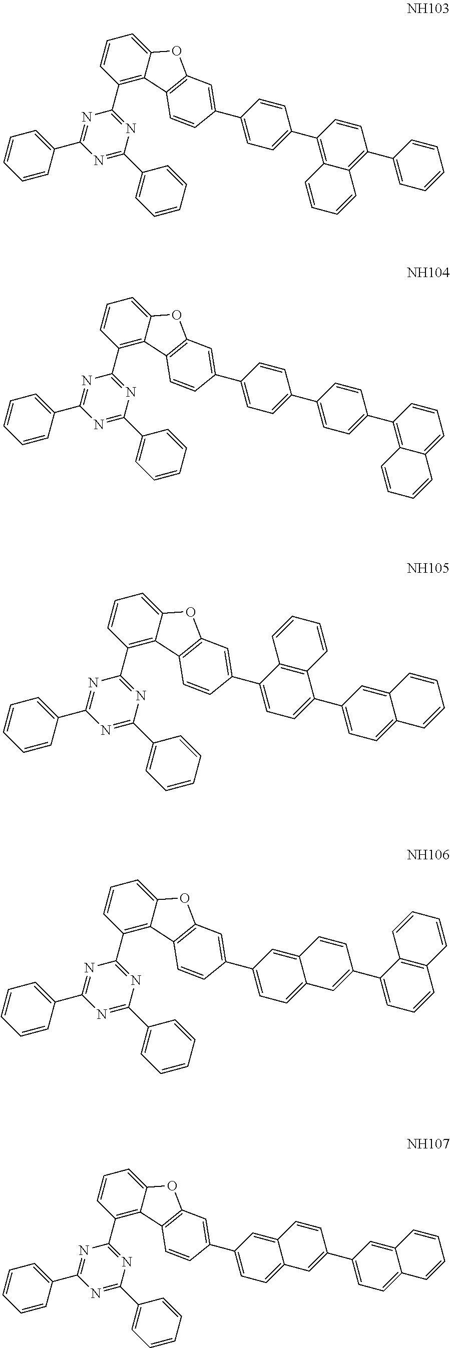 [SMILES: c1ccc(-c2nc(-c3ccccc3)nc(-c3cccc4oc5cc(-c6ccc(-c7ccc(-c8ccccc8)c8ccccc78)cc6)ccc5c34)n2)cc1 ; c1ccc(-c2nc(-c3ccccc3)nc(-c3cccc4oc5cc(-c6ccc(-c7ccc(-c8cccc9ccccc89)cc7)cc6)ccc5c34)n2)cc1 ; c1ccc(-c2nc(-c3ccccc3)nc(-c3cccc4oc5cc(-c6ccc(-c7ccc8ccccc8c7)c7ccccc67)ccc5c34)n2)cc1 ; c1ccc(-c2nc(-c3ccccc3)nc(-c3cccc4oc5cc(-c6ccc7cc(-c8cccc9ccccc89)ccc7c6)ccc5c34)n2)cc1 ; c1ccc(-c2nc(-c3ccccc3)nc(-c3cccc4oc5cc(-c6ccc7cc(-c8ccc9ccccc9c8)ccc7c6)ccc5c34)n2)cc1]

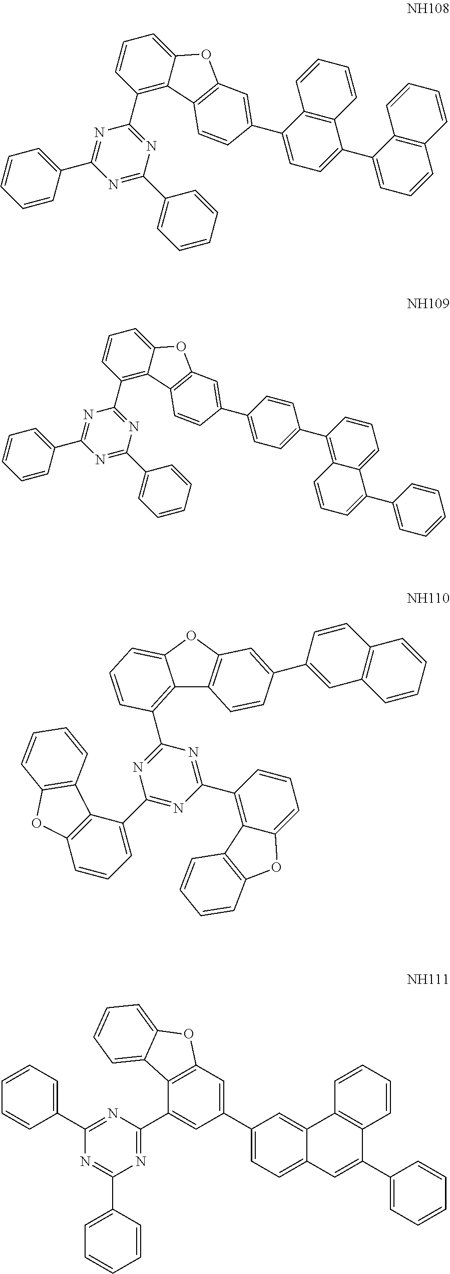 [SMILES: c1ccc(-c2nc(-c3ccccc3)nc(-c3cccc4oc5cc(-c6ccc(-c7cccc8ccccc78)c7ccccc67)ccc5c34)n2)cc1 ; c1ccc(-c2nc(-c3ccccc3)nc(-c3cccc4oc5cc(-c6ccc(-c7cccc8c(-c9ccccc9)cccc78)cc6)ccc5c34)n2)cc1 ; c1ccc2cc(-c3ccc4c(c3)oc3cccc(-c5nc(-c6cccc7oc8ccccc8c67)nc(-c6cccc7oc8ccccc8c67)n5)c34)ccc2c1 ; c1ccc(-c2nc(-c3ccccc3)nc(-c3cc(-c4ccc5cc(-c6ccccc6)c6ccccc6c5c4)cc4oc5ccccc5c34)n2)cc1]

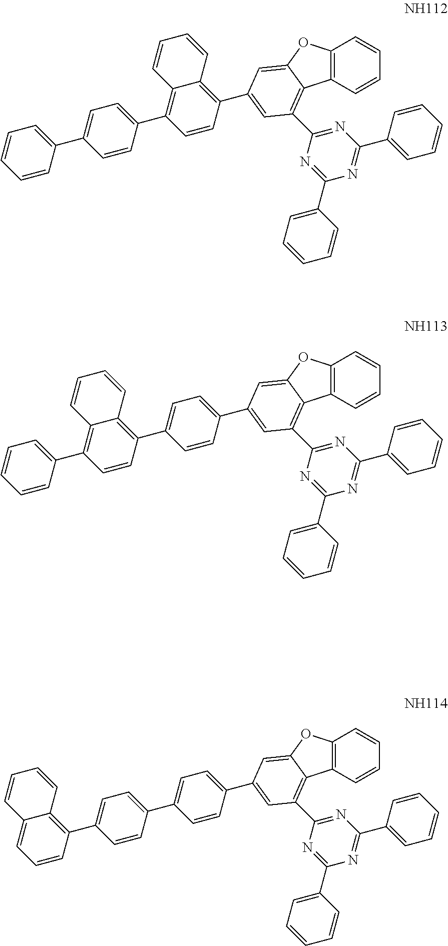 [SMILES: c1ccc(-c2ccc(-c3ccc(-c4cc(-c5nc(-c6ccccc6)nc(-c6ccccc6)n5)c5c(c4)oc4ccccc45)c4ccccc34)cc2)cc1 ; c1ccc(-c2nc(-c3ccccc3)nc(-c3cc(-c4ccc(-c5ccc(-c6ccccc6)c6ccccc56)cc4)cc4oc5ccccc5c34)n2)cc1 ; c1ccc(-c2nc(-c3ccccc3)nc(-c3cc(-c4ccc(-c5ccc(-c6cccc7ccccc67)cc5)cc4)cc4oc5ccccc5c34)n2)cc1]

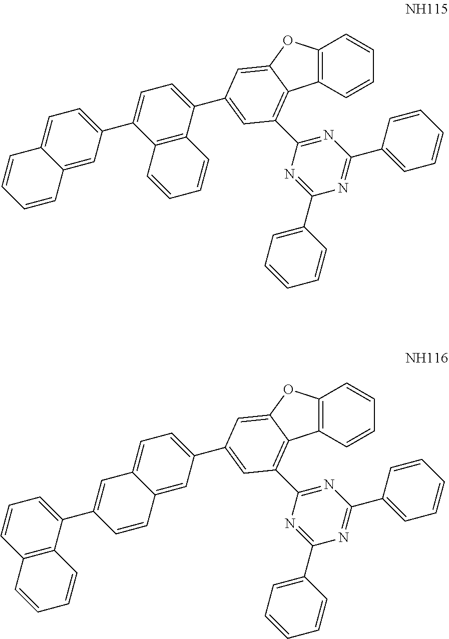 [SMILES: c1ccc(-c2nc(-c3ccccc3)nc(-c3cc(-c4ccc(-c5ccc6ccccc6c5)c5ccccc45)cc4oc5ccccc5c34)n2)cc1 ; c1ccc(-c2nc(-c3ccccc3)nc(-c3cc(-c4ccc5cc(-c6cccc7ccccc67)ccc5c4)cc4oc5ccccc5c34)n2)cc1]

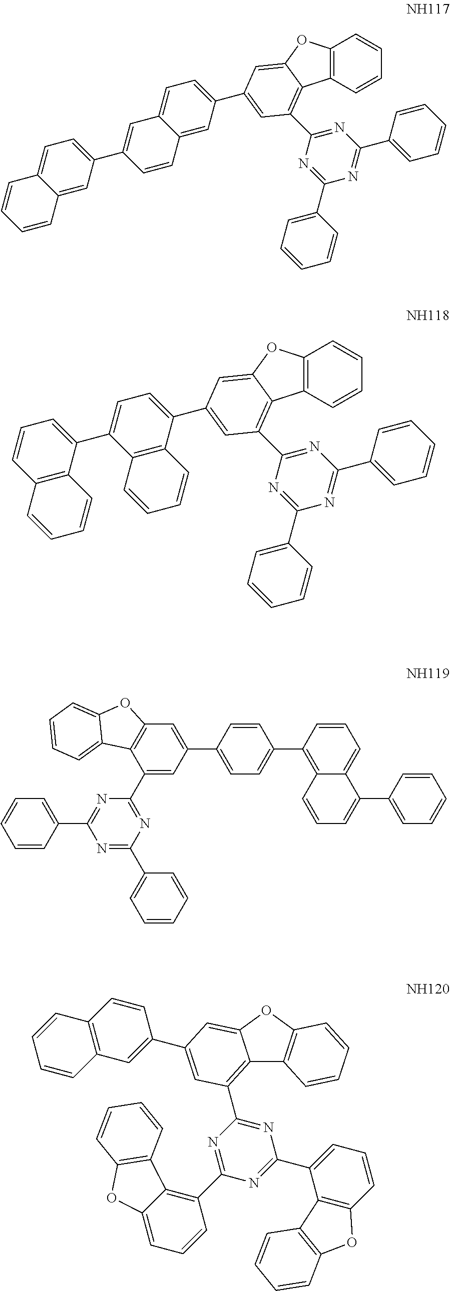 [SMILES: c1ccc(-c2nc(-c3ccccc3)nc(-c3cc(-c4ccc5cc(-c6ccc7ccccc7c6)ccc5c4)cc4oc5ccccc5c34)n2)cc1 ; c1ccc(-c2nc(-c3ccccc3)nc(-c3cc(-c4ccc(-c5cccc6ccccc56)c5ccccc45)cc4oc5ccccc5c34)n2)cc1 ; c1ccc(-c2nc(-c3ccccc3)nc(-c3cc(-c4ccc(-c5cccc6c(-c7ccccc7)cccc56)cc4)cc4oc5ccccc5c34)n2)cc1 ; c1ccc2cc(-c3cc(-c4nc(-c5cccc6oc7ccccc7c56)nc(-c5cccc6oc7ccccc7c56)n4)c4c(c3)oc3ccccc34)ccc2c1]

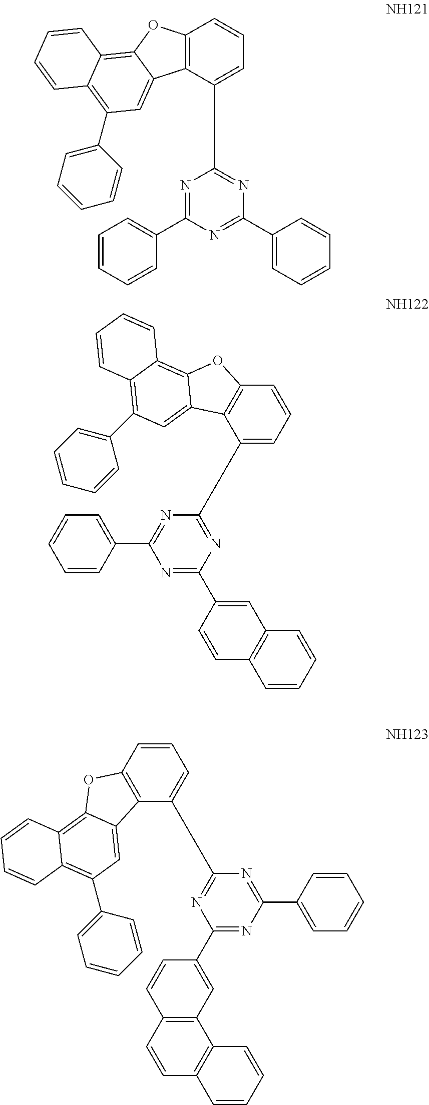

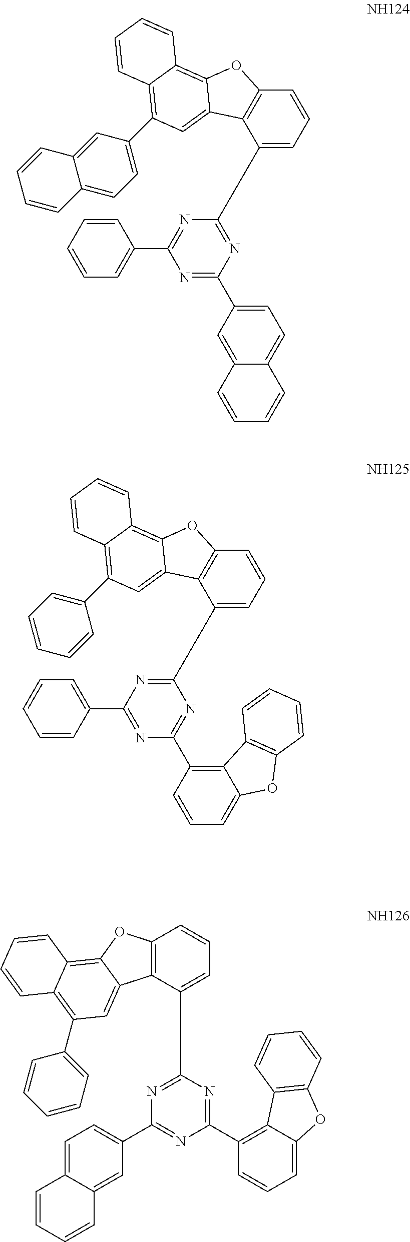 [SMILES: c1ccc(-c2nc(-c3ccc4ccccc4c3)nc(-c3cccc4oc5c6ccccc6c(-c6ccc7ccccc7c6)cc5c34)n2)cc1 ; c1ccc(-c2nc(-c3cccc4oc5ccccc5c34)nc(-c3cccc4oc5c6ccccc6c(-c6ccccc6)cc5c34)n2)cc1 ; c1ccc(-c2cc3c(oc4cccc(-c5nc(-c6ccc7ccccc7c6)nc(-c6cccc7oc8ccccc8c67)n5)c43)c3ccccc23)cc1]

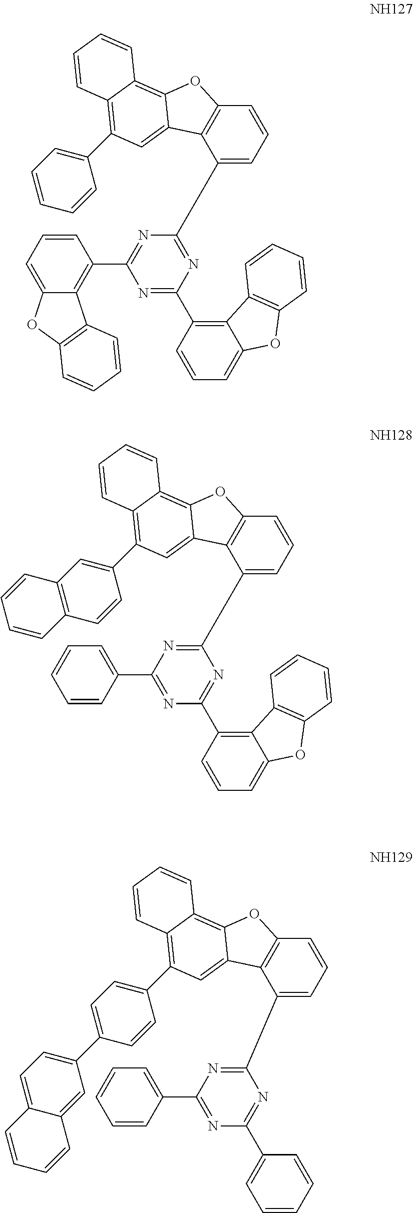 [SMILES: c1ccc(-c2cc3c(oc4cccc(-c5nc(-c6cccc7oc8ccccc8c67)nc(-c6cccc7oc8ccccc8c67)n5)c43)c3ccccc23)cc1 ; c1ccc(-c2nc(-c3cccc4oc5ccccc5c34)nc(-c3cccc4oc5c6ccccc6c(-c6ccc7ccccc7c6)cc5c34)n2)cc1 ; c1ccc(-c2nc(-c3ccccc3)nc(-c3cccc4oc5c6ccccc6c(-c6ccc(-c7ccc8ccccc8c7)cc6)cc5c34)n2)cc1]

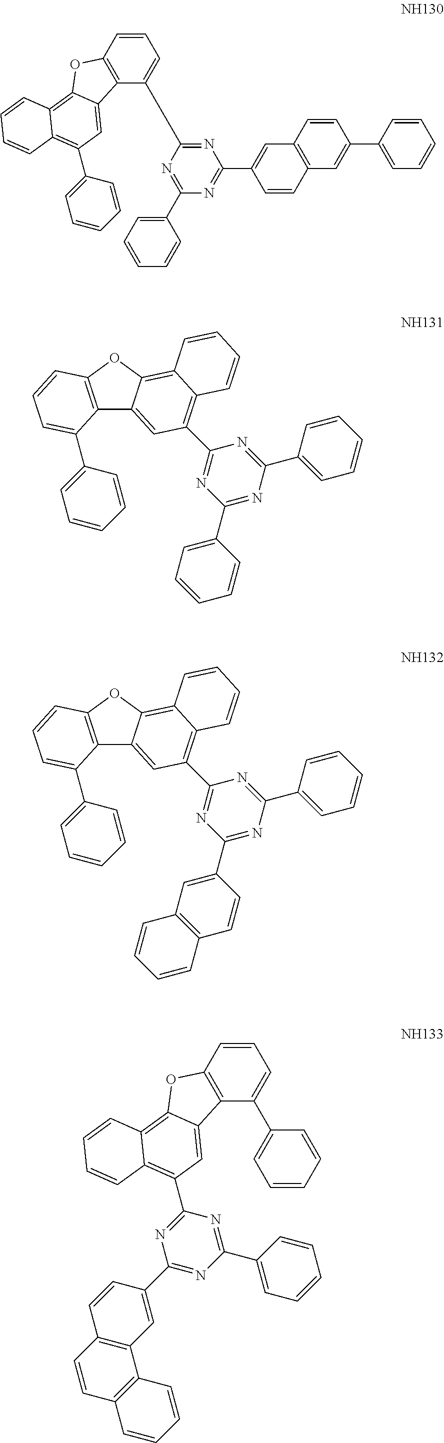 [SMILES: c1ccc(-c2ccc3cc(-c4nc(-c5ccccc5)nc(-c5cccc6oc7c8ccccc8c(-c8ccccc8)cc7c56)n4)ccc3c2)cc1 ; c1ccc(-c2nc(-c3ccccc3)nc(-c3cc4c(oc5cccc(-c6ccccc6)c54)c4ccccc34)n2)cc1 ; c1ccc(-c2nc(-c3ccc4ccccc4c3)nc(-c3cc4c(oc5cccc(-c6ccccc6)c54)c4ccccc34)n2)cc1 ; c1ccc(-c2nc(-c3ccc4ccc5ccccc5c4c3)nc(-c3cc4c(oc5cccc(-c6ccccc6)c54)c4ccccc34)n2)cc1]

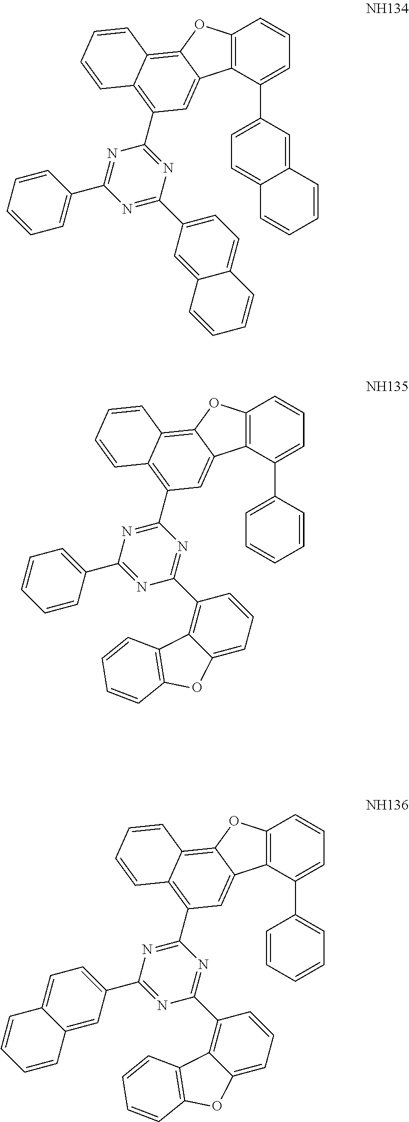 [SMILES: c1ccc(-c2nc(-c3ccc4ccccc4c3)nc(-c3cc4c(oc5cccc(-c6ccc7ccccc7c6)c54)c4ccccc34)n2)cc1 ; c1ccc(-c2nc(-c3cc4c(oc5cccc(-c6ccccc6)c54)c4ccccc34)nc(-c3cccc4oc5ccccc5c34)n2)cc1 ; c1ccc(-c2cccc3oc4c5ccccc5c(-c5nc(-c6ccc7ccccc7c6)nc(-c6cccc7oc8ccccc8c67)n5)cc4c23)cc1]

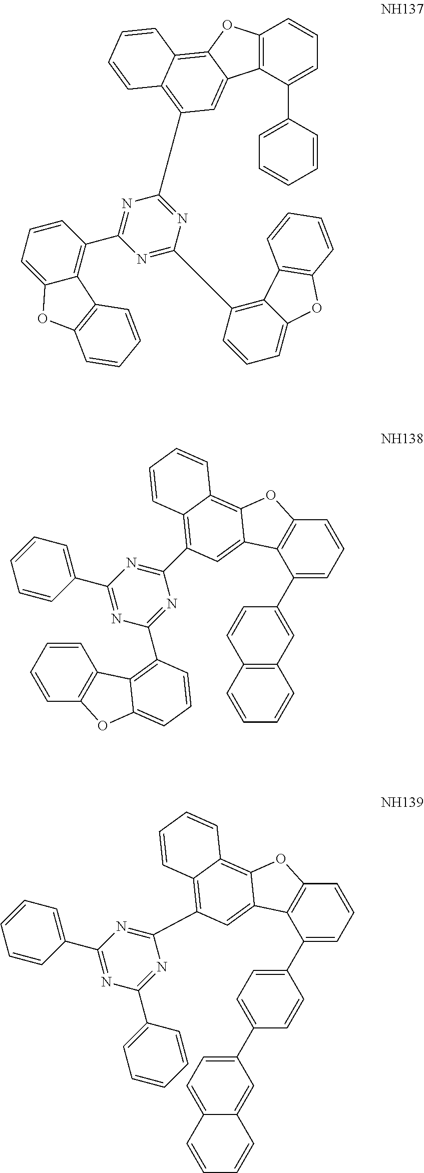 [SMILES: c1ccc(-c2cccc3oc4c5ccccc5c(-c5nc(-c6cccc7oc8ccccc8c67)nc(-c6cccc7oc8ccccc8c67)n5)cc4c23)cc1 ; c1ccc(-c2nc(-c3cc4c(oc5cccc(-c6ccc7ccccc7c6)c54)c4ccccc34)nc(-c3cccc4oc5ccccc5c34)n2)cc1 ; c1ccc(-c2nc(-c3ccccc3)nc(-c3cc4c(oc5cccc(-c6ccc(-c7ccc8ccccc8c7)cc6)c54)c4ccccc34)n2)cc1]

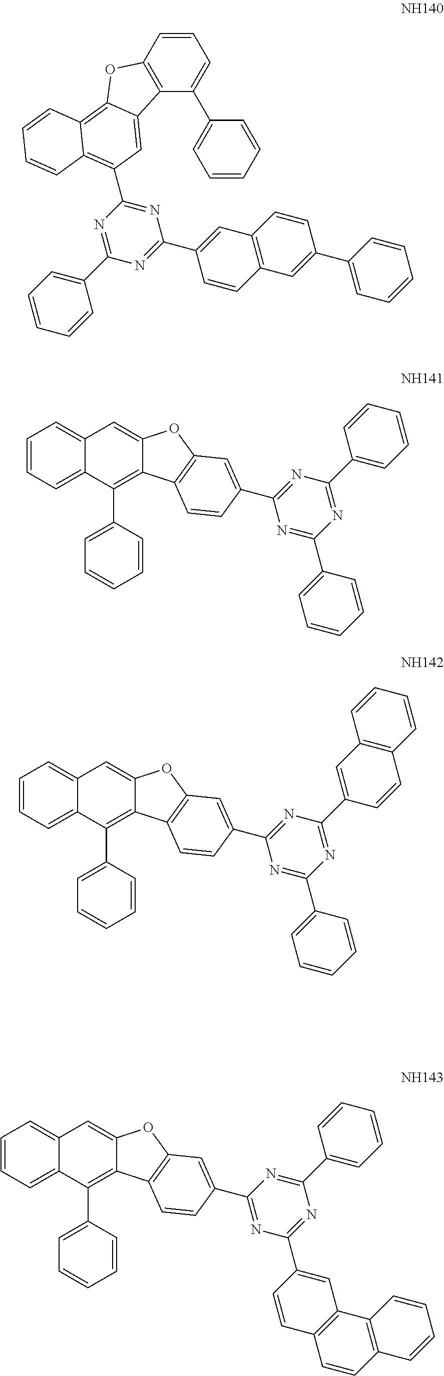 [SMILES: c1ccc(-c2ccc3cc(-c4nc(-c5ccccc5)nc(-c5cc6c(oc7cccc(-c8ccccc8)c76)c6ccccc56)n4)ccc3c2)cc1 ; c1ccc(-c2nc(-c3ccccc3)nc(-c3ccc4c(c3)oc3cc5ccccc5c(-c5ccccc5)c34)n2)cc1 ; c1ccc(-c2nc(-c3ccc4ccccc4c3)nc(-c3ccc4c(c3)oc3cc5ccccc5c(-c5ccccc5)c34)n2)cc1 ; c1ccc(-c2nc(-c3ccc4c(c3)oc3cc5ccccc5c(-c5ccccc5)c34)nc(-c3ccc4ccc5ccccc5c4c3)n2)cc1]

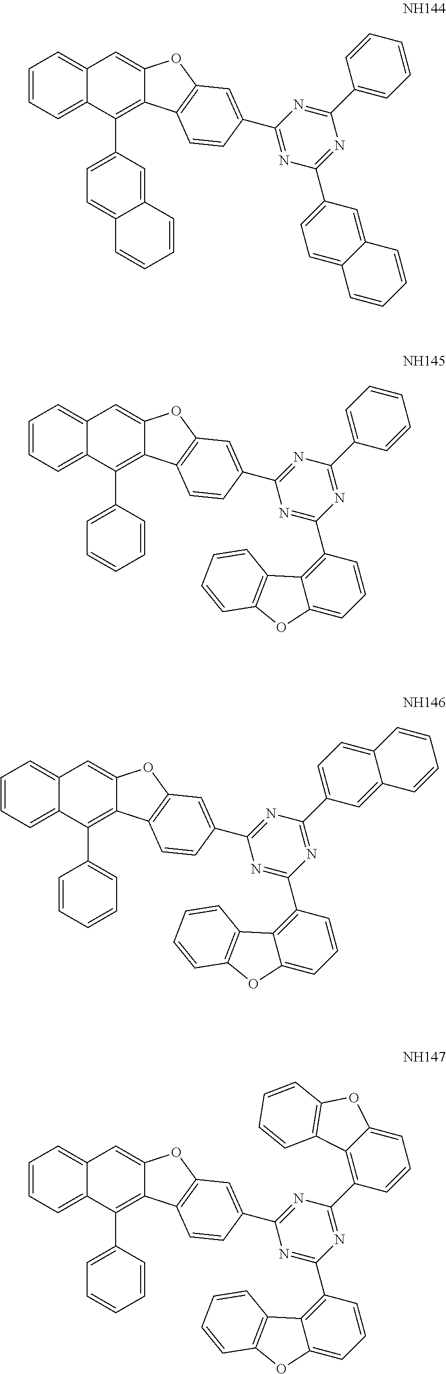 [SMILES: c1ccc(-c2nc(-c3ccc4ccccc4c3)nc(-c3ccc4c(c3)oc3cc5ccccc5c(-c5ccc6ccccc6c5)c34)n2)cc1 ; c1ccc(-c2nc(-c3ccc4c(c3)oc3cc5ccccc5c(-c5ccccc5)c34)nc(-c3cccc4oc5ccccc5c34)n2)cc1 ; c1ccc(-c2c3ccccc3cc3oc4cc(-c5nc(-c6ccc7ccccc7c6)nc(-c6cccc7oc8ccccc8c67)n5)ccc4c23)cc1 ; c1ccc(-c2c3ccccc3cc3oc4cc(-c5nc(-c6cccc7oc8ccccc8c67)nc(-c6cccc7oc8ccccc8c67)n5)ccc4c23)cc1]

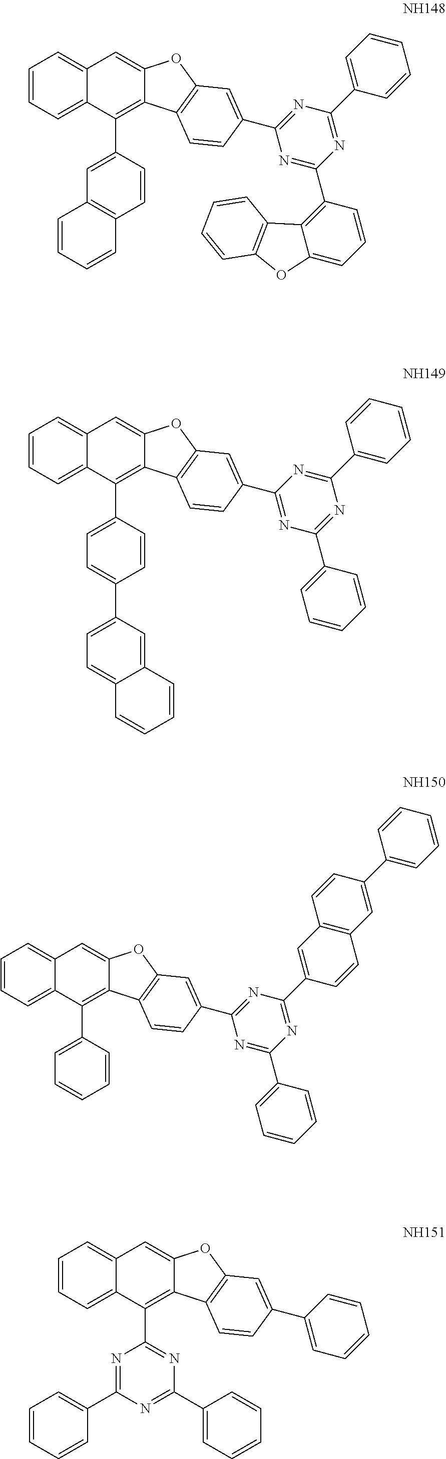 [SMILES: c1ccc(-c2nc(-c3ccc4c(c3)oc3cc5ccccc5c(-c5ccc6ccccc6c5)c34)nc(-c3cccc4oc5ccccc5c34)n2)cc1 ; c1ccc(-c2nc(-c3ccccc3)nc(-c3ccc4c(c3)oc3cc5ccccc5c(-c5ccc(-c6ccc7ccccc7c6)cc5)c34)n2)cc1 ; c1ccc(-c2ccc3cc(-c4nc(-c5ccccc5)nc(-c5ccc6c(c5)oc5cc7ccccc7c(-c7ccccc7)c56)n4)ccc3c2)cc1 ; c1ccc(-c2ccc3c(c2)oc2cc4ccccc4c(-c4nc(-c5ccccc5)nc(-c5ccccc5)n4)c23)cc1]

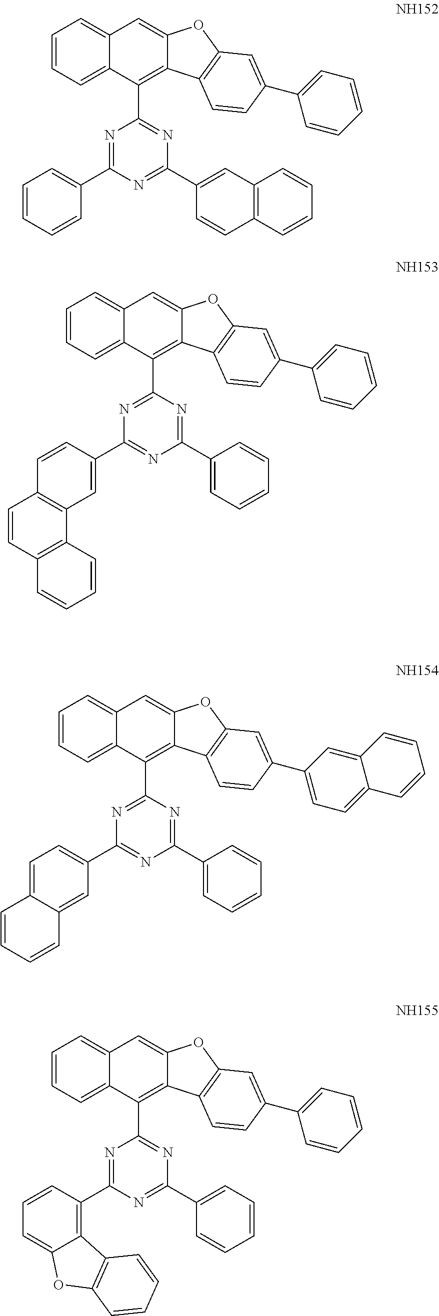 [SMILES: c1ccc(-c2ccc3c(c2)oc2cc4ccccc4c(-c4nc(-c5ccccc5)nc(-c5ccc6ccccc6c5)n4)c23)cc1 ; c1ccc(-c2ccc3c(c2)oc2cc4ccccc4c(-c4nc(-c5ccccc5)nc(-c5ccc6ccc7ccccc7c6c5)n4)c23)cc1 ; c1ccc(-c2nc(-c3ccc4ccccc4c3)nc(-c3c4ccccc4cc4oc5cc(-c6ccc7ccccc7c6)ccc5c34)n2)cc1 ; c1ccc(-c2ccc3c(c2)oc2cc4ccccc4c(-c4nc(-c5ccccc5)nc(-c5cccc6oc7ccccc7c56)n4)c23)cc1]

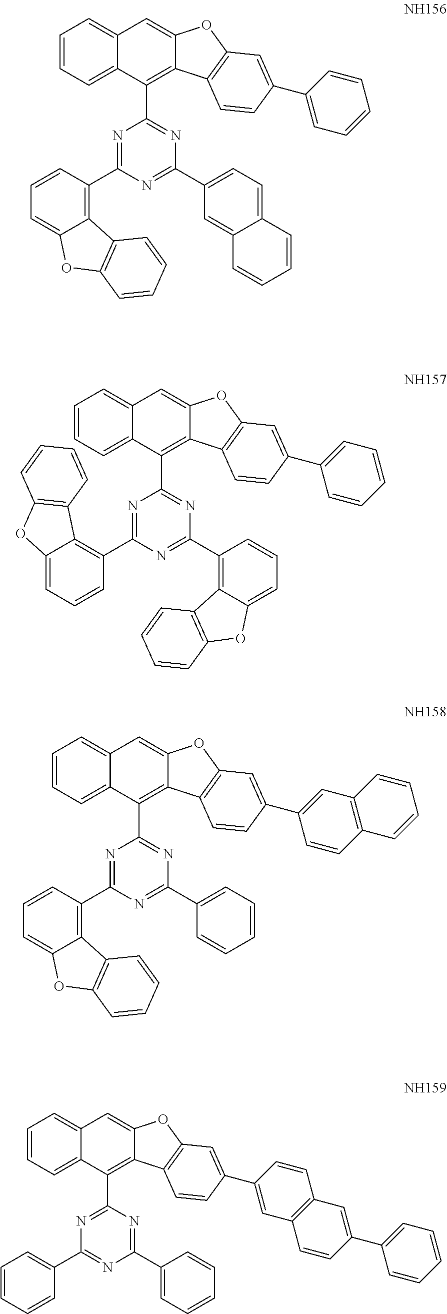 [SMILES: c1ccc(-c2ccc3c(c2)oc2cc4ccccc4c(-c4nc(-c5ccc6ccccc6c5)nc(-c5cccc6oc7ccccc7c56)n4)c23)cc1 ; c1ccc(-c2ccc3c(c2)oc2cc4ccccc4c(-c4nc(-c5cccc6oc7ccccc7c56)nc(-c5cccc6oc7ccccc7c56)n4)c23)cc1 ; c1ccc(-c2nc(-c3cccc4oc5ccccc5c34)nc(-c3c4ccccc4cc4oc5cc(-c6ccc7ccccc7c6)ccc5c34)n2)cc1 ; c1ccc(-c2ccc3cc(-c4ccc5c(c4)oc4cc6ccccc6c(-c6nc(-c7ccccc7)nc(-c7ccccc7)n6)c45)ccc3c2)cc1]

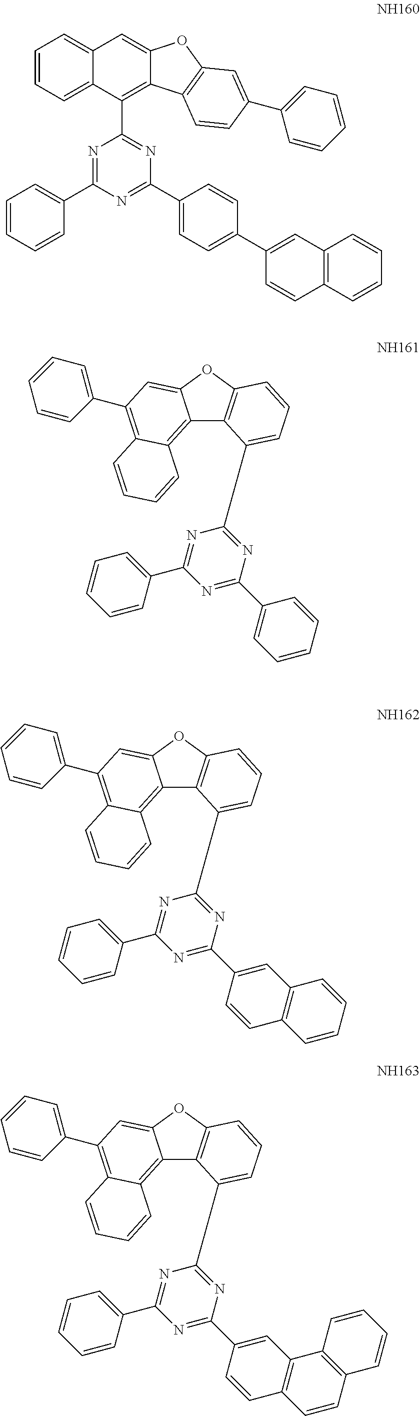 [SMILES: c1ccc(-c2ccc3c(c2)oc2cc4ccccc4c(-c4nc(-c5ccccc5)nc(-c5ccc(-c6ccc7ccccc7c6)cc5)n4)c23)cc1 ; c1ccc(-c2nc(-c3ccccc3)nc(-c3cccc4oc5cc(-c6ccccc6)c6ccccc6c5c34)n2)cc1 ; c1ccc(-c2nc(-c3ccc4ccccc4c3)nc(-c3cccc4oc5cc(-c6ccccc6)c6ccccc6c5c34)n2)cc1 ; c1ccc(-c2nc(-c3ccc4ccc5ccccc5c4c3)nc(-c3cccc4oc5cc(-c6ccccc6)c6ccccc6c5c34)n2)cc1]

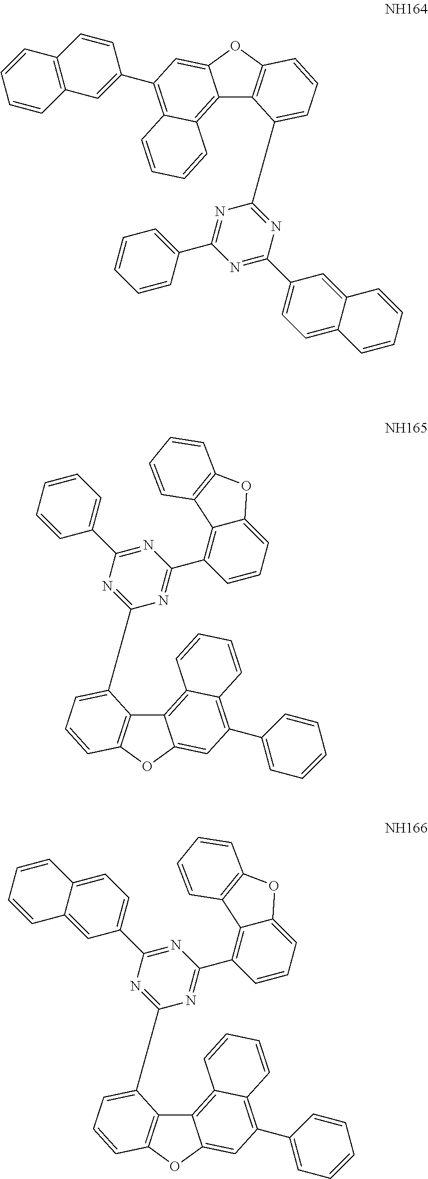 [SMILES: c1ccc(-c2nc(-c3ccc4ccccc4c3)nc(-c3cccc4oc5cc(-c6ccc7ccccc7c6)c6ccccc6c5c34)n2)cc1 ; c1ccc(-c2nc(-c3cccc4oc5ccccc5c34)nc(-c3cccc4oc5cc(-c6ccccc6)c6ccccc6c5c34)n2)cc1 ; c1ccc(-c2cc3oc4cccc(-c5nc(-c6ccc7ccccc7c6)nc(-c6cccc7oc8ccccc8c67)n5)c4c3c3ccccc23)cc1]

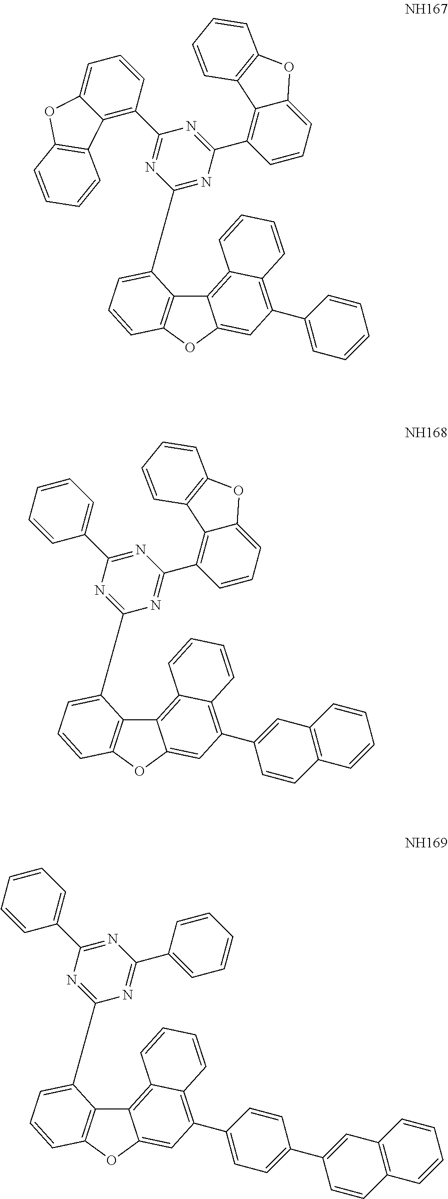 [SMILES: c1ccc(-c2cc3oc4cccc(-c5nc(-c6cccc7oc8ccccc8c67)nc(-c6cccc7oc8ccccc8c67)n5)c4c3c3ccccc23)cc1 ; c1ccc(-c2nc(-c3cccc4oc5ccccc5c34)nc(-c3cccc4oc5cc(-c6ccc7ccccc7c6)c6ccccc6c5c34)n2)cc1 ; c1ccc(-c2nc(-c3ccccc3)nc(-c3cccc4oc5cc(-c6ccc(-c7ccc8ccccc8c7)cc6)c6ccccc6c5c34)n2)cc1]

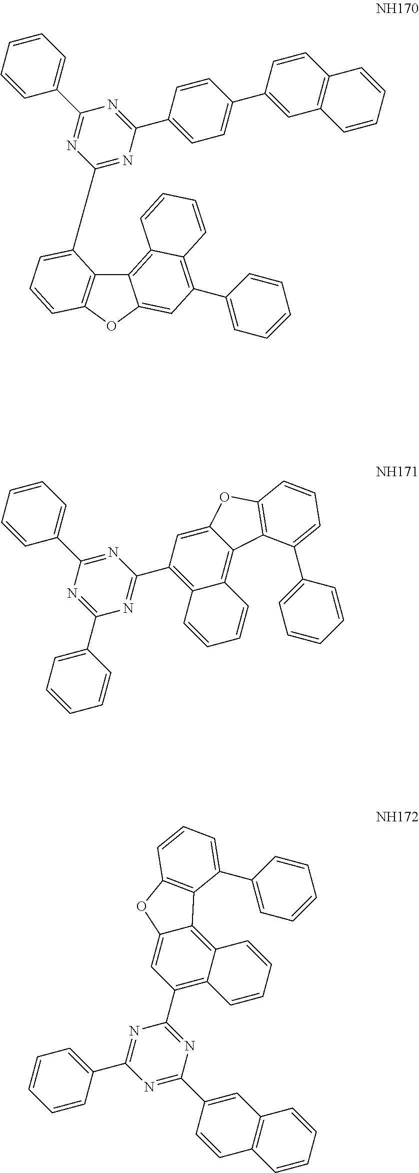 [SMILES: c1ccc(-c2nc(-c3ccc(-c4ccc5ccccc5c4)cc3)nc(-c3cccc4oc5cc(-c6ccccc6)c6ccccc6c5c34)n2)cc1 ; c1ccc(-c2nc(-c3ccccc3)nc(-c3cc4oc5cccc(-c6ccccc6)c5c4c4ccccc34)n2)cc1 ; c1ccc(-c2nc(-c3ccc4ccccc4c3)nc(-c3cc4oc5cccc(-c6ccccc6)c5c4c4ccccc34)n2)cc1]

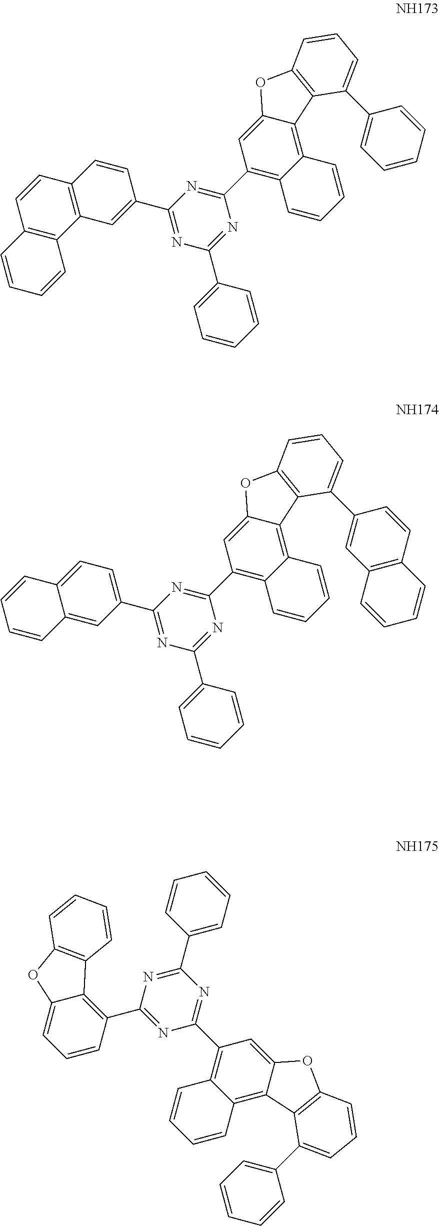 [SMILES: c1ccc(-c2nc(-c3ccc4ccc5ccccc5c4c3)nc(-c3cc4oc5cccc(-c6ccccc6)c5c4c4ccccc34)n2)cc1 ; c1ccc(-c2nc(-c3ccc4ccccc4c3)nc(-c3cc4oc5cccc(-c6ccc7ccccc7c6)c5c4c4ccccc34)n2)cc1 ; c1ccc(-c2nc(-c3cc4oc5cccc(-c6ccccc6)c5c4c4ccccc34)nc(-c3cccc4oc5ccccc5c34)n2)cc1]

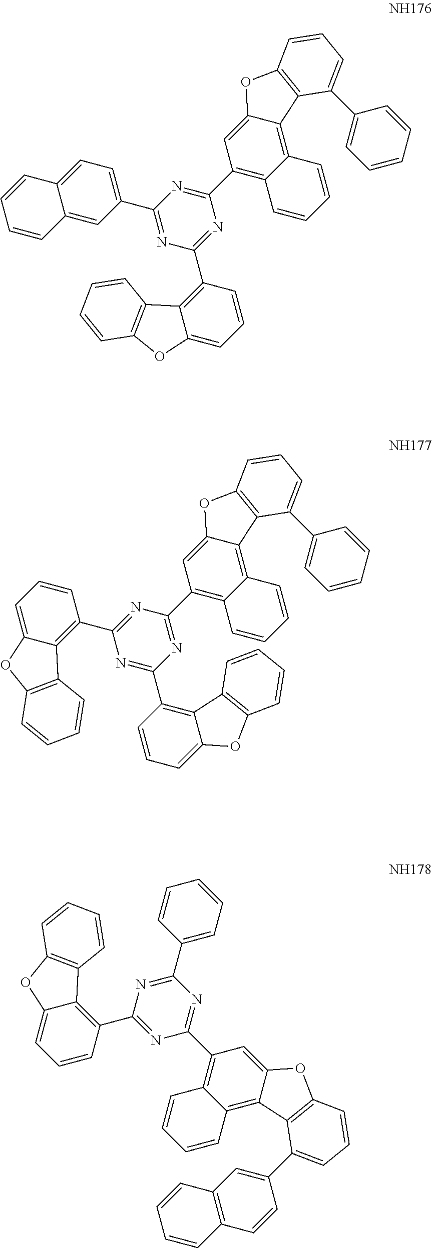 [SMILES: c1ccc(-c2cccc3oc4cc(-c5nc(-c6ccc7ccccc7c6)nc(-c6cccc7oc8ccccc8c67)n5)c5ccccc5c4c23)cc1 ; c1ccc(-c2cccc3oc4cc(-c5nc(-c6cccc7oc8ccccc8c67)nc(-c6cccc7oc8ccccc8c67)n5)c5ccccc5c4c23)cc1 ; c1ccc(-c2nc(-c3cc4oc5cccc(-c6ccc7ccccc7c6)c5c4c4ccccc34)nc(-c3cccc4oc5ccccc5c34)n2)cc1]

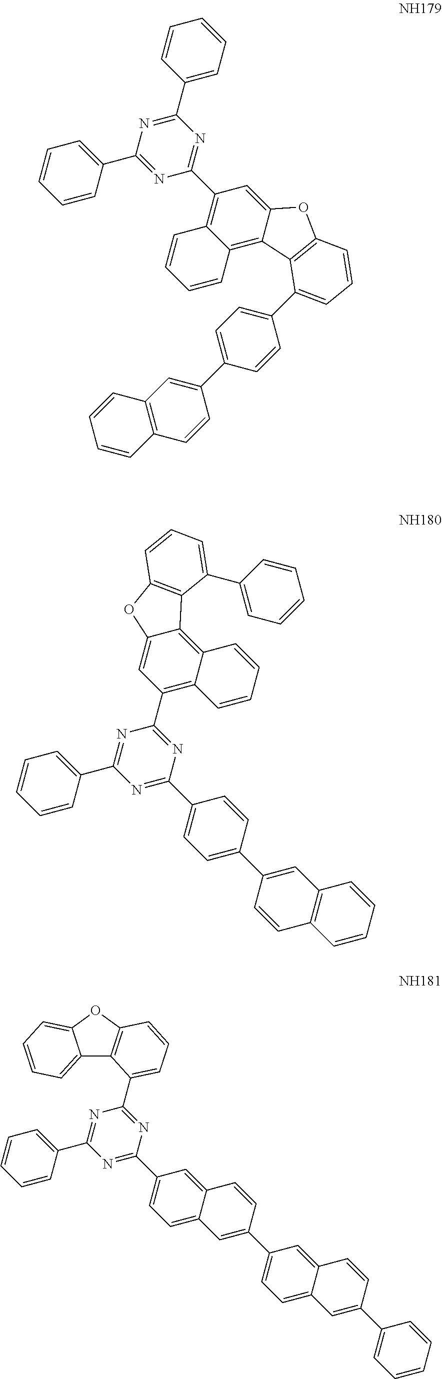 [SMILES: c1ccc(-c2nc(-c3ccccc3)nc(-c3cc4oc5cccc(-c6ccc(-c7ccc8ccccc8c7)cc6)c5c4c4ccccc34)n2)cc1 ; c1ccc(-c2nc(-c3ccc(-c4ccc5ccccc5c4)cc3)nc(-c3cc4oc5cccc(-c6ccccc6)c5c4c4ccccc34)n2)cc1 ; c1ccc(-c2ccc3cc(-c4ccc5cc(-c6nc(-c7ccccc7)nc(-c7cccc8oc9ccccc9c78)n6)ccc5c4)ccc3c2)cc1]

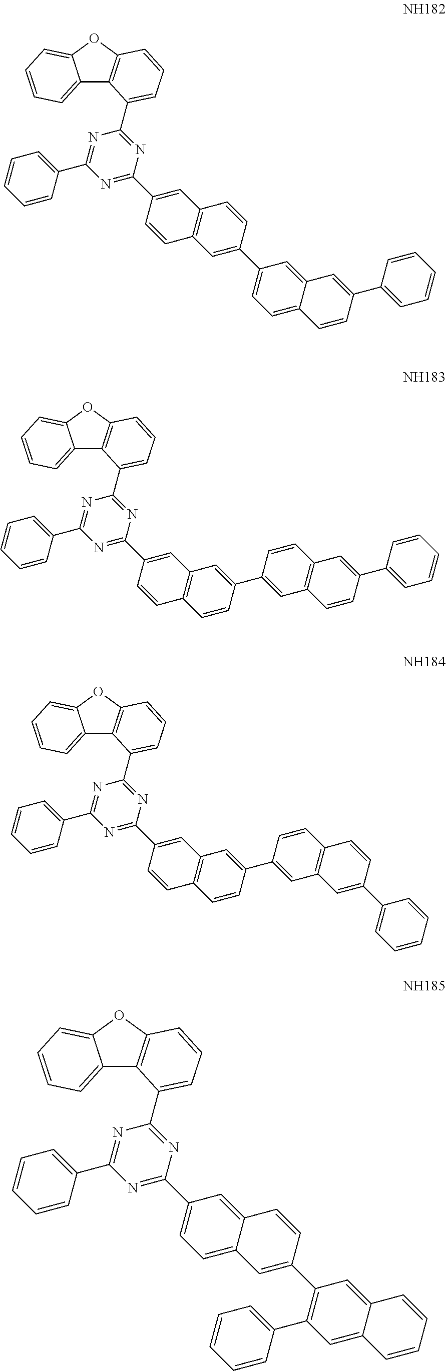 [SMILES: c1ccc(-c2ccc3ccc(-c4ccc5cc(-c6nc(-c7ccccc7)nc(-c7cccc8oc9ccccc9c78)n6)ccc5c4)cc3c2)cc1 ; c1ccc(-c2ccc3cc(-c4ccc5ccc(-c6nc(-c7ccccc7)nc(-c7cccc8oc9ccccc9c78)n6)cc5c4)ccc3c2)cc1 ; c1ccc(-c2ccc3ccc(-c4ccc5ccc(-c6nc(-c7ccccc7)nc(-c7cccc8oc9ccccc9c78)n6)cc5c4)cc3c2)cc1 ; c1ccc(-c2nc(-c3ccc4cc(-c5cc6ccccc6cc5-c5ccccc5)ccc4c3)nc(-c3cccc4oc5ccccc5c34)n2)cc1]

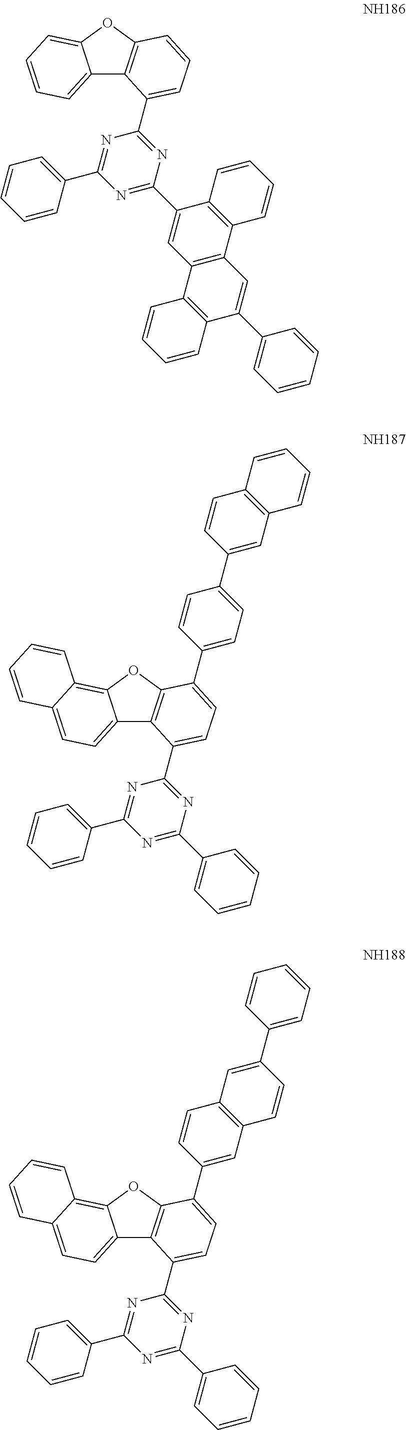 [SMILES: c1ccc(-c2nc(-c3cc4c5ccccc5c(-c5ccccc5)cc4c4ccccc34)nc(-c3cccc4oc5ccccc5c34)n2)cc1 ; c1ccc(-c2nc(-c3ccccc3)nc(-c3ccc(-c4ccc(-c5ccc6ccccc6c5)cc4)c4oc5c6ccccc6ccc5c34)n2)cc1 ; c1ccc(-c2ccc3cc(-c4ccc(-c5nc(-c6ccccc6)nc(-c6ccccc6)n5)c5c4oc4c6ccccc6ccc45)ccc3c2)cc1]

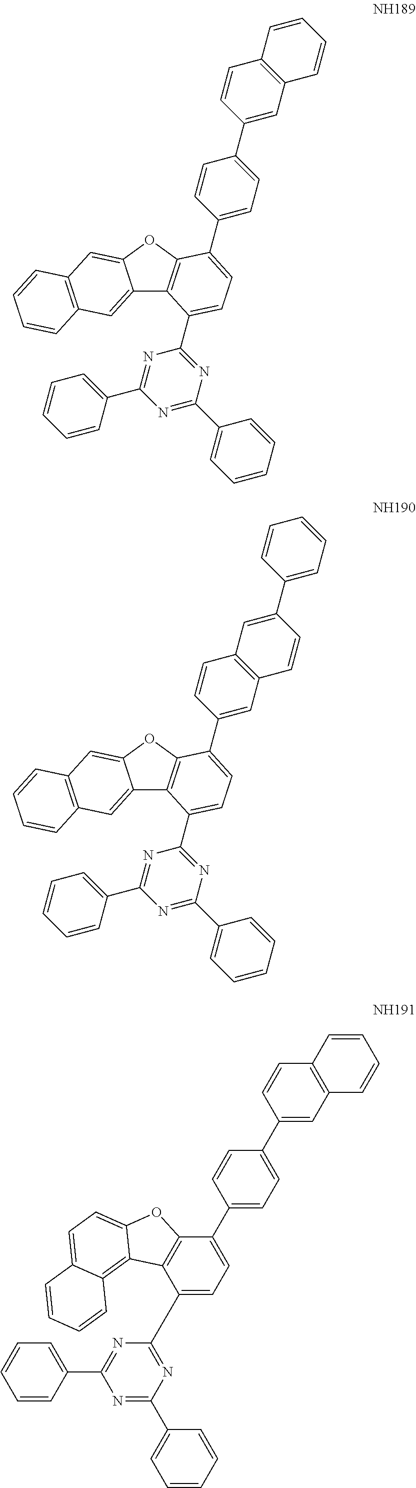 [SMILES: c1ccc(-c2nc(-c3ccccc3)nc(-c3ccc(-c4ccc(-c5ccc6ccccc6c5)cc4)c4oc5cc6ccccc6cc5c34)n2)cc1 ; c1ccc(-c2ccc3cc(-c4ccc(-c5nc(-c6ccccc6)nc(-c6ccccc6)n5)c5c4oc4cc6ccccc6cc45)ccc3c2)cc1 ; c1ccc(-c2nc(-c3ccccc3)nc(-c3ccc(-c4ccc(-c5ccc6ccccc6c5)cc4)c4oc5ccc6ccccc6c5c34)n2)cc1]

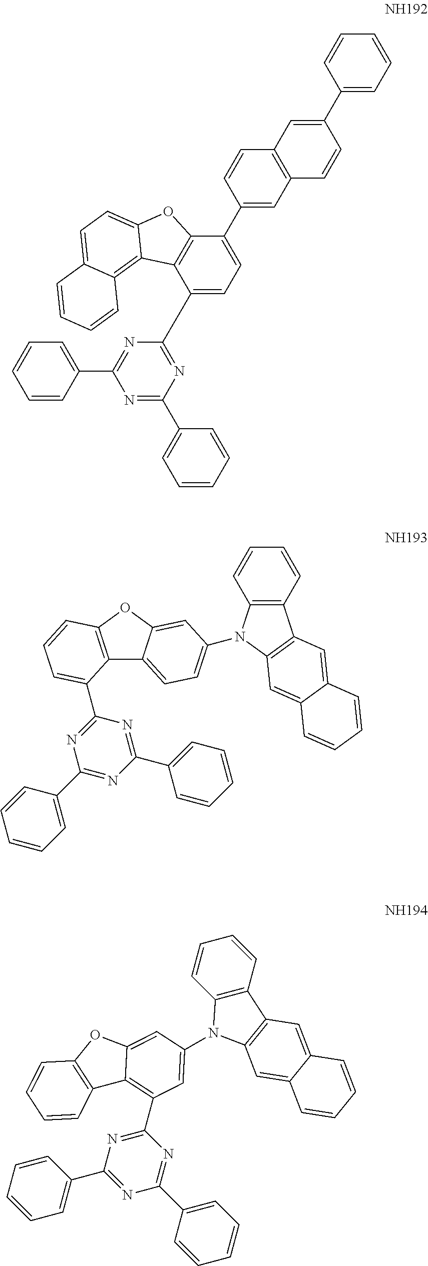 [SMILES: c1ccc(-c2ccc3cc(-c4ccc(-c5nc(-c6ccccc6)nc(-c6ccccc6)n5)c5c4oc4ccc6ccccc6c45)ccc3c2)cc1 ; c1ccc(-c2nc(-c3ccccc3)nc(-c3cccc4oc5cc(-n6c7ccccc7c7cc8ccccc8cc76)ccc5c34)n2)cc1 ; c1ccc(-c2nc(-c3ccccc3)nc(-c3cc(-n4c5ccccc5c5cc6ccccc6cc54)cc4oc5ccccc5c34)n2)cc1]

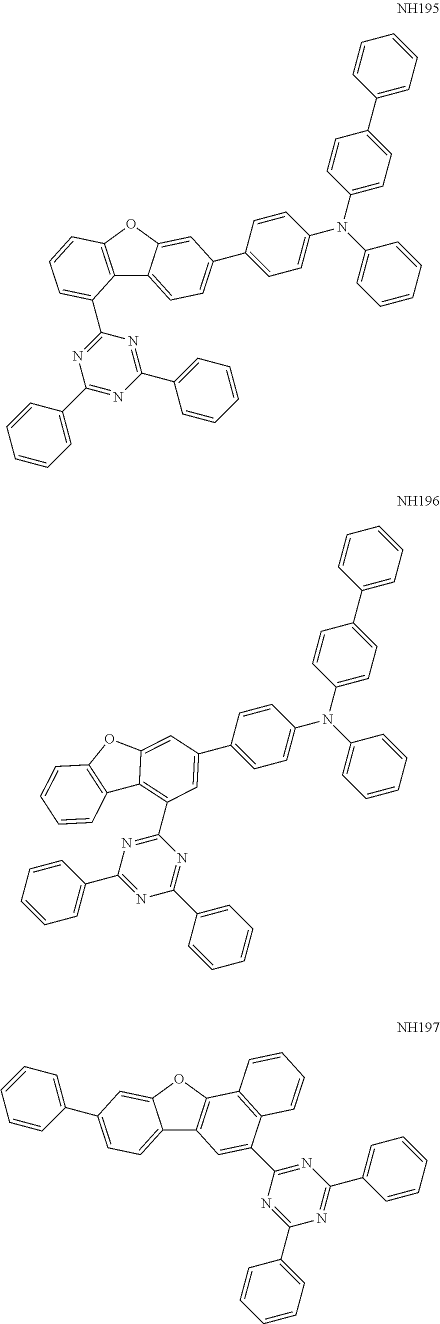 [SMILES: c1ccc(-c2ccc(N(c3ccccc3)c3ccc(-c4ccc5c(c4)oc4cccc(-c6nc(-c7ccccc7)nc(-c7ccccc7)n6)c45)cc3)cc2)cc1 ; c1ccc(-c2ccc(N(c3ccccc3)c3ccc(-c4cc(-c5nc(-c6ccccc6)nc(-c6ccccc6)n5)c5c(c4)oc4ccccc45)cc3)cc2)cc1 ; c1ccc(-c2ccc3c(c2)oc2c4ccccc4c(-c4nc(-c5ccccc5)nc(-c5ccccc5)n4)cc32)cc1]

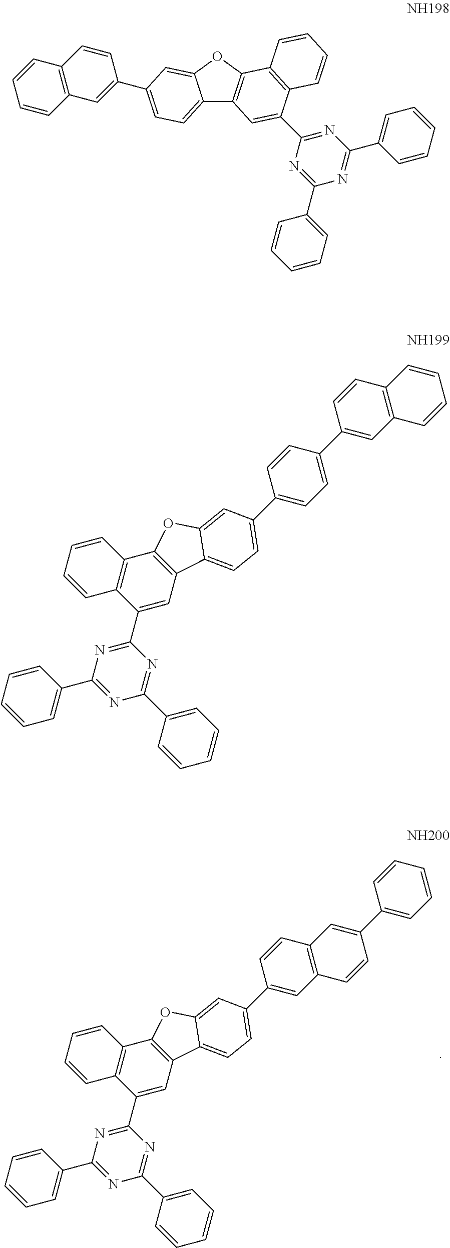 [SMILES: c1ccc(-c2nc(-c3ccccc3)nc(-c3cc4c5ccc(-c6ccc7ccccc7c6)cc5oc4c4ccccc34)n2)cc1 ; c1ccc(-c2nc(-c3ccccc3)nc(-c3cc4c5ccc(-c6ccc(-c7ccc8ccccc8c7)cc6)cc5oc4c4ccccc34)n2)cc1 ; c1ccc(-c2ccc3cc(-c4ccc5c(c4)oc4c6ccccc6c(-c6nc(-c7ccccc7)nc(-c7ccccc7)n6)cc54)ccc3c2)cc1]

15. The organic light emitting device of claim 11, wherein the organic material layer comprises a light emitting layer, and the light emitting layer comprises the heterocyclic compound of claim 1 and the heterocyclic compound represented by Chemical Formula 2.

16. The organic light emitting device of claim 7, further comprising one, or two or more layers selected from the group consisting of a light emitting layer, a hole injection layer, a hole transport layer, an electron injection layer, an electron transport layer, an electron blocking layer and a hole blocking layer.

17. A composition for an organic material layer, the composition comprising:
the heterocyclic compound of claim 1; and
a heterocyclic compound represented by the following Chemical Formula 2: 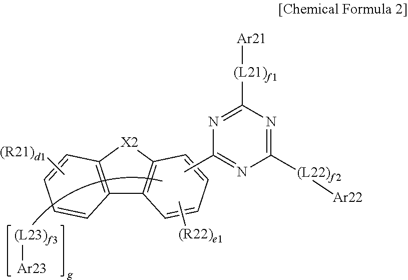
wherein, in Chemical Formula 2,
X2 is O; or S;
R21 and R22 are the same as or different from each other, and each independently selected from the group consisting of hydrogen; deuterium; halogen; a cyano group; a substituted or unsubstituted C1 to C60 alkyl group; a substituted or unsubstituted C2 to C60 alkenyl group; a substituted or unsubstituted C2 to C60 alkynyl group; a substituted or unsubstituted C1 to C60 alkoxy group; a substituted or unsubstituted C3 to C60 cycloalkyl group; a substituted or unsubstituted C2 to C60 heterocycloalkyl group; —P(═O)R201R202; —SiR201R202R203; and —NR201R202, or two or more groups adjacent to each other bond to each other to form a substituted or unsubstituted C6 to C60 aromatic hydrocarbon ring; or a substituted or unsubstituted C2 to C60 heteroring, and R201, R202 and R203 are the same as or different from each other and each independently a substituted or unsubstituted C1 to C60 alkyl group; a substituted or unsubstituted C6 to C60 aryl group; or a substituted or unsubstituted C2 to C60 heteroaryl group;
d1 is an integer of 0 to 4, and when d1 is 2 or greater, R21s are the same as or different from each other;
e1 is an integer of 0 to 3, and when e1 is 2 or greater, R22s are the same as or different from each other;
L21 to L23 are the same as or different from each other, and each independently a direct bond;
a substituted or unsubstituted C6 to C60 arylene group; or a substituted or unsubstituted C2 to C60 heteroarylene group;
f1 is an integer of 0 to 5, and when f1 is 2 or greater, L21s are the same as or different from each other;
f2 is an integer of 0 to 5, and when f2 is 2 or greater, L22s are the same as or different from each other;
f3 is an integer of 0 to 5, and when f3 is 2 or greater, L23s are the same as or different from each other;
Ar21 to Ar23 are the same as or different from each other, and each independently a substituted or unsubstituted C6 to C60 aryl group; a substituted or unsubstituted C2 to C60 heteroaryl group; or —NAr24Ar25;
Ar24 and Ar25 are the same as or different from each other, and each independently a substituted or unsubstituted C6 to C60 aryl group; or a substituted or unsubstituted C2 to C60 heteroaryl group; and
g is 0 or 1.

18. The composition of claim 17, wherein the heterocyclic compound represented by Chemical Formula 1 and the heterocyclic compound represented by Chemical Formula 2 have a weight ratio of 1:9 to 9:1.
